(19) United States
(12) Patent Application Publication
(10) Pub. No.: US 2025/0057037 A1
(43) Pub. Date: Feb. 13, 2025

(54) LIGHT-EMITTING DEVICE INCLUDING AMINE-BASED COMPOUND, ELECTRONIC APPARATUS AND ELECTRONIC DEVICE EACH INCLUDING THE LIGHT-EMITTING DEVICE, AND THE AMINE-BASED COMPOUND

(71) Applicant: Samsung Display Co., Ltd., Yongin-si (KR)

(72) Inventors: Minji Kim, Yongin-si (KR); Beomjoon Kim, Yongin-si (KR); Chaeyeong Kim, Yongin-si (KR); Byeongwook Yoo, Yongin-si (KR); Sanghyun Han, Yongin-si (KR)

(73) Assignee: Samsung Display Co., Ltd., Yongin-si (KR)

(21) Appl. No.: 18/735,428

(22) Filed: Jun. 6, 2024

(30) Foreign Application Priority Data
Jun. 19, 2023 (KR) 10-2023-0078353

(51) Int. Cl. H10K 85/60; C07B 59/00; G02F 1/1335; H10K 50/15; H10K 85/30; H10K 85/40; H10K 101/20

(52) U.S. Cl. CPC H10K 85/633; C07B 59/004; G02F 1/133514; H10K 50/15; H10K 85/342; H10K 85/346; H10K 85/348; H10K 85/40; H10K 85/615; H10K 85/654; H10K 85/6572; H10K 85/6574; H10K 85/6576; H10K 85/658; C07B 2200/05; H10K 2101/20

(57) ABSTRACT

Embodiments provide an amine-based compound, a light-emitting device including the amine-based compound, an electronic apparatus including the light-emitting device, and an electronic device including the light-emitting device. The light-emitting device includes a first electrode, a second electrode facing the first electrode, and an interlayer between the first electrode and the second electrode, wherein the interlayer includes an emission layer and a hole transport region between the first electrode and the emission layer, and the hole transport region includes an amine-based compound, which is represented by Formula 1, wherein Formula 1 is explained in the specification:

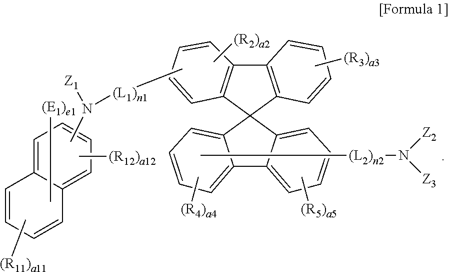

### CROSS-REFERENCE TO RELATED APPLICATION(S)

This application claims priority to and benefits of Korean Patent Application No. 10-2023-0078353 under 35 U.S.C. § 119, filed on Jun. 19, 2023 in the Korean Intellectual Property Office, the entire contents of which are incorporated herein by reference.

### BACKGROUND

### 1. Technical Field

The disclosure relates to a light-emitting device including an amine-based compound, an electronic apparatus and an electronic device each including the light-emitting device, and the amine-based compound.

### 2. Description of the Related Art

Among light-emitting devices, organic light-emitting devices are self-emissive devices that have wide viewing angles, high contrast ratios, short response times, and excellent characteristics in terms of luminance, driving voltage, and response speed, compared to devices in the related art.

As an example, an organic light-emitting device may have a structure in which a first electrode is arranged on a substrate, and a hole transport region, an emission layer, an electron transport region, and a second electrode are sequentially formed on the first electrode. Holes provided from the first electrode move toward the emission layer through the hole transport region, and electrons provided from the second electrode move toward the emission layer through the electron transport region. Carriers, such as holes and electrons, recombine in the emission layer to produce excitons. The excitons may transition from an excited state to a ground state, thereby generating light.

It is to be understood that this background of the technology section is, in part, intended to provide useful background for understanding the technology. However, this background of the technology section may also include ideas, concepts, or recognitions that were not part of what was known or appreciated by those skilled in the pertinent art prior to a corresponding effective filing date of the subject matter disclosed herein.

### SUMMARY

Embodiments include a light-emitting device including a novel amine-based compound, an electronic apparatus and an electronic device each including the light-emitting device, and the amine-based compound.

Additional aspects will be set forth in part in the description which follows and, in part, will be apparent from the description, or may be learned by practice of the embodiments.

According to embodiments, a light-emitting device may include a first electrode, a second electrode facing the first electrode, an interlayer between the first electrode and the second electrode, wherein
the interlayer may include an emission layer, and a hole transport region between the first electrode and the emission layer, and
the hole transport region may include an amine-based compound represented by Formula 1:

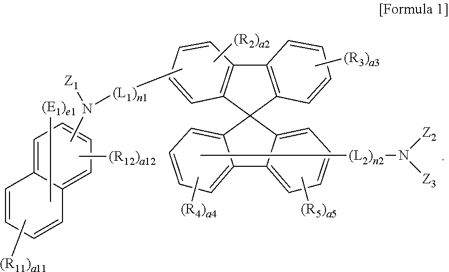

In Formula 1,

L₁ and L₂ may each independently be a single bond, a C₅-C₆₀ carbocyclic group unsubstituted or substituted with at least one R₁₀ₐ, or a C₁-C₆₀ heterocyclic group unsubstituted or substituted with at least one R₁₀ₐ,
n1 and n2 may each independently be an integer from 1 to 3,
Z₁ to Z₃ and E may each independently be deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
e1 may be an integer from 1 to 7,
R₁₁, R₁₂, and R₂ to R₅ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
a11 may be an integer from 0 to 4,
a12 may be an integer from 0 to 3,
a2 may be an integer from 0 to 3,
a3 to a5 may each independently be an integer from 0 to 4,
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a C₃-C₆₀ carbocyclic group, a C₁-C₆₀ heterocyclic group, or any combination thereof.

In an embodiment, the hole transport region may include a hole injection layer, a hole transport layer, an emission auxiliary layer, an electron blocking layer, or any combination thereof, and at least one of the hole transport layer and the hole injection layer may include the amine-based compound.

In an embodiment, the light-emitting device may further include a second compound including at least one π electron-deficient nitrogen-containing C₁-C₆₀ heterocyclic group, a third compound including a group represented by Formula 3, which is explained herein, a fourth compound that may be a delayed fluorescence compound, a fifth compound that is a transition metal-containing organometallic compound, or any combination thereof, wherein the second compound, the third compound, the fourth compound, and the fifth compound may be different from each other, and Formula 3 is explained below.

In an embodiment, the second compound may include a pyridine group, a pyrimidine group, a pyrazine group, a pyridazine group, a triazine group, or any combination thereof.

In an embodiment, the fourth compound may include at least one cyclic group including boron (B) and nitrogen (N) as ring-forming atoms.

In an embodiment, the fifth compound may include a platinum atom and a tetradentate ligand bonded to the platinum atom; and one of bonds between the platinum atom and the tetradentate ligand may be a platinum-carbene bond.

In an embodiment, the fifth compound may include an iridium atom and a tetradentate ligand bonded to the iridium atom; and one of bonds between the iridium atom and the tetradentate ligand may be an iridium-carbene bond.

In an embodiment, the emission layer may include the second compound, the third compound, the fourth compound, the fifth compound, or a combination thereof; and the emission layer may emit blue light.

According to embodiments, an electronic apparatus may include the light-emitting device.

In an embodiment, the electronic apparatus may further include a color filter, a color conversion layer, a touch screen layer, a polarizing layer, or any combination thereof.

In an embodiment, the electronic apparatus may further include a thin-film transistor, wherein the thin-film transistor may include a source electrode and a drain electrode, and the first electrode of the light-emitting device may be electrically connected to the source electrode or the drain electrode.

According to embodiments, an electronic device may include the light-emitting device.

In an embodiment, the electronic device may be a flat panel display, a curved display, a computer monitor, a medical monitor, a television (TV), an advertisement board, an indoor light, an outdoor light, a signal light, a head-up display, a fully transparent display, a partially transparent display, a flexible display, a rollable display, a foldable display, a stretchable display, a laser printer, a telephone, a portable phone, a tablet personal computer, a phablet, a personal digital assistant (PDA), a wearable device, a laptop computer, a digital camera, a camcorder, a viewfinder, a microdisplay, a three-dimensional (3D) display, a virtual reality display, an augmented reality display, a vehicle, a video wall with multiple displays tiled together, a theater screen, a stadium screen, a phototherapy device, or a sign.

According to embodiments, an amine-based compound may be represented by Formula 1, which is explained herein.

In an embodiment, Z₁ to Z₃ and E₁ may each independently be a benzene group, a naphthalene group, a fluorene group, a carbazole group, a dibenzofuran group, a dibenzothiophene group, an azacarbazole group, an azadibenzofuran group, an azadibenzothiophene group, a pyridine group, a pyrimidine group, a pyrazine group, a quinoline group, or a quinoxaline group, each unsubstituted or substituted with at least one R₁₀ₐ.

In an embodiment, Z₁ to Z₃ and E₁ may each independently be a group represented by one of Formulae 1A-1 to 1A-16, which are explained below.

In an embodiment, in Formula 1, a moiety represented by

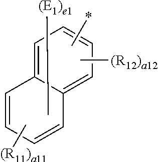

may be a moiety represented by one of Formulae 1B-1 to 1B-14, which are explained below.

In an embodiment, Z₂ and Z₃ may be different from each other.

In an embodiment, the amine-based compound may be represented by Formula 1-1, which is explained below.

In an embodiment, the amine-based compound may be one of Compounds 1 to 206, which are explained below.

It is to be understood that the embodiments above are described in a generic and explanatory sense only and not for the purpose of limitation, and the disclosure is not limited to the embodiments described above.

### BRIEF DESCRIPTION OF THE DRAWINGS

The above and other aspects and features of the disclosure will be more apparent by describing in detail embodiments thereof with reference to the accompanying drawings, in which:

FIG. 1 is a schematic cross-sectional view of a light-emitting device according to an embodiment;

FIG. 2 is a schematic cross-sectional view of an electronic apparatus according to an embodiment;

FIG. 3 is a schematic cross-sectional view of an electronic apparatus according to another embodiment;

FIG. 4 is a schematic perspective view of an electronic device including a light-emitting device according to an embodiment;

FIG. 5 is a schematic perspective view of the exterior of a vehicle as an electronic device including a light-emitting device according to an embodiment; and

FIG. 6A to FIG. 6C are each a schematic diagram of the interior of a vehicle, according to embodiments.

### DETAILED DESCRIPTION OF THE EMBODIMENTS

The disclosure will now be described more fully hereinafter with reference to the accompanying drawings, in which embodiments are shown. This disclosure may, however, be embodied in different forms and should not be construed as limited to the embodiments set forth herein. Rather, these embodiments are provided so that this disclosure will be thorough and complete, and will fully convey the scope of the disclosure to those skilled in the art.

In the drawings, the sizes, thicknesses, ratios, and dimensions of the elements may be exaggerated for ease of description and for clarity. Like reference numbers and reference characters refer to like elements throughout.

In the specification, it will be understood that when an element (or region, layer, part, etc.) is referred to as being “on”, “connected to”, or “coupled to” another element, it can be directly on, connected to, or coupled to the other element, or one or more intervening elements may be present therebetween. In a similar sense, when an element (or region, layer, part, etc.) is described as “covering” another element, it can directly cover the other element, or one or more intervening elements may be present therebetween.

In the specification, when an element is “directly on,” “directly connected to,” or “directly coupled to” another element, there are no intervening elements present. For example, “directly on” may mean that two layers or two elements are disposed without an additional element such as an adhesion element therebetween.

In the specification, the expressions used in the singular such as “a,” “an,” and “the,” are intended to include the plural forms as well, unless the context clearly indicates otherwise.

In the specification, the term “and/or” includes any and all combinations of one or more of the associated listed items. For example, “A and/or B” may be understood to mean “A, B, or A and B.” The terms “and” and “or” may be used in the conjunctive or disjunctive sense and may be understood to be equivalent to “and/or”.

In the specification and the claims, the term “at least one of” is intended to include the meaning of “at least one selected from the group consisting of” for the purpose of its meaning and interpretation. For example, “at least one of A, B, and C” may be understood to mean A only, B only, C only, or any combination of two or more of A, B, and C, such as ABC, ACC, BC, or CC. When preceding a list of elements, the term, “at least one of,” modifies the entire list of elements and does not modify the individual elements of the list.

It will be understood that, although the terms first, second, etc. may be used herein to describe various elements, these elements should not be limited by these terms. These terms are only used to distinguish one element from another element. Thus, a first element could be termed a second element without departing from the teachings of the disclosure. Similarly, a second element could be termed a first element, without departing from the scope of the disclosure.

The spatially relative terms “below”, “beneath”, “lower”, “above”, “upper”, or the like, may be used herein for ease of description to describe the relations between one element or component and another element or component as illustrated in the drawings. It will be understood that the spatially relative terms are intended to encompass different orientations of the device in use or operation, in addition to the orientation depicted in the drawings. For example, in the case where a device illustrated in the drawing is turned over, the device positioned “below” or “beneath” another device may be placed “above” another device. Accordingly, the illustrative term “below” may include both the lower and upper positions. The device may also be oriented in other directions and thus the spatially relative terms may be interpreted differently depending on the orientations.

The terms “about” or “approximately” as used herein is inclusive of the stated value and means within an acceptable range of deviation for the recited value as determined by one of ordinary skill in the art, considering the measurement in question and the error associated with measurement of the recited quantity (i.e., the limitations of the measurement system). For example, “about” may mean within one or more standard deviations, or within ±20%, ±10%, or ±5% of the stated value.

It should be understood that the terms “comprises,” “comprising,” “includes,” “including,” “have,” “having,” “contains,” “containing,” and the like are intended to specify the presence of stated features, integers, steps, operations, elements, components, or combinations thereof in the disclosure, but do not preclude the presence or addition of one or more other features, integers, steps, operations, elements, components, or combinations thereof.

Unless otherwise defined or implied herein, all terms (including technical and scientific terms) used have the same meaning as commonly understood by those skilled in the art to which this disclosure pertains. It will be further understood that terms, such as those defined in commonly used dictionaries, should be interpreted as having a meaning that is consistent with their meaning in the context of the relevant art and should not be interpreted in an ideal or excessively formal sense unless clearly defined in the specification.

Embodiments provide a light-emitting device (e.g., an organic light-emitting device) which may include: a first electrode; a second electrode facing the first electrode; an interlayer between the first electrode and the second electrode, wherein

the interlayer may include an emission layer; and a hole transport region between the first electrode and the emission layer, and the hole transport region may include an amine-based compound represented by Formula 1:

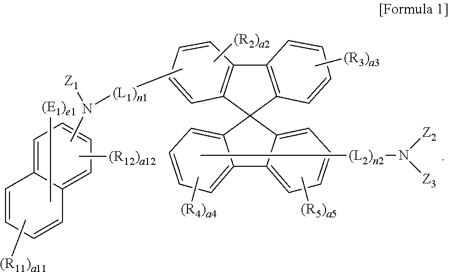

Details on Formula 1 are the same as described herein.

In an embodiment, the first electrode of the light-emitting device may be an anode, the second electrode of the light-emitting device may be a cathode,

the interlayer may further include an electron transport region between the emission layer and the second electrode,

the hole transport region may include a hole injection layer, a hole transport layer, an emission auxiliary layer, an electron blocking layer, or any combination thereof, and

the electron transport region may include a hole blocking layer, an electron transport layer, an electron injection layer, or any combination thereof.

In an embodiment, at least one of the hole transport layer and the hole injection layer may include the amine-based compound represented by Formula 1. Accordingly, the amine-based compound represented by Formula 1 may be used as a hole transport material.

In another embodiment, the emission layer of the light-emitting device may include the amine-based compound represented by Formula 1.

In an embodiment, the light-emitting device may further include a second compound including at least one IT electron-deficient nitrogen-containing C₁-C₆₀ heterocyclic group, a third compound including a group represented by Formula 3, a fourth compound capable of emitting delayed fluorescence (e.g., a delayed fluorescence compound), a fifth compound that is a transition metal-containing organometallic compound, or any combination thereof, and

the second compound, the third compound, the fourth compound, and the fifth compound may be different from each other:

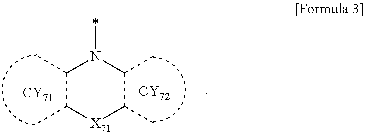

In Formula 3,
ring CY₇₁ and ring CY₇₂ may each independently be a π electron-rich C₃-C₆₀ cyclic group or a pyridine group,
X₇₁ may be: a single bond; or a linking group including O, S, N, B, C, Si, or any combination thereof, and
* indicates a binding site to any atom included in the remaining part other than Formula 3 in the third compound.

In an embodiment, the second compound, the third compound, the fourth compound, and the fifth compound may each include at least one deuterium atom.

In another embodiment, the second compound may include at least one silicon atom.

In another embodiment, the third compound may include at least one silicon atom.

In embodiments, the light-emitting device may further include the second compound and the third compound; and at least one of the second compound and the third compound may each independently include at least one deuterium atom, at least one silicon atom, or any combination thereof.

In an embodiment, the light-emitting device (e.g., the emission layer of the light-emitting device) may further include the second compound. The second compound may include at least one deuterium atom. For example, the light-emitting device (e.g., the emission layer of the light-emitting device) may further include, in addition to the second compound, the third compound, the fourth compound, the fifth compound, or any combination thereof.

In another embodiment, the light-emitting device (e.g., the emission layer of the light-emitting device) may further include the third compound. The third compound may include at least one deuterium atom. For example, the light-emitting device (e.g., the emission layer of the light-emitting device) may further include, in addition to the third compound, the second compound, the fourth compound, the fifth compound, or any combination thereof.

In another embodiment, the light-emitting device (e.g., the emission layer of the light-emitting device) may further include the fourth compound. The fourth compound may include at least one deuterium atom. The fourth compound may improve color purity, luminescence efficiency, and lifespan characteristics of the light-emitting device. For example, the light-emitting device (e.g., the emission layer of the light-emitting device) may further include, in addition to the fourth compound, the second compound, the third compound, the fifth compound, or any combination thereof.

In another embodiment, the light-emitting device (e.g., the emission layer of the light-emitting device) may further include the fifth compound. The fifth compound may include at least one deuterium atom. For example, the light-emitting device (e.g., the emission layer of the light-emitting device) may further include, in addition to the fifth compound, the second compound, the third compound, the fourth compound, or any combination thereof.

In an embodiment, the emission layer of the light-emitting device may include the second compound, the third compound, the fourth compound, the fifth compound, or any combination thereof; and the emission layer may emit blue light.

According to an embodiment, the emission layer may emit light having a maximum emission wavelength in a range of about 400 nm to about 490 nm.

In an embodiment, the blue light may be deep blue light.

In an embodiment, the second compound may include a pyridine group, a pyrimidine group, a pyrazine group, a pyridazine group, a triazine group, or any combination thereof.

In an embodiment, the third compound may exclude CBP and mCBP:

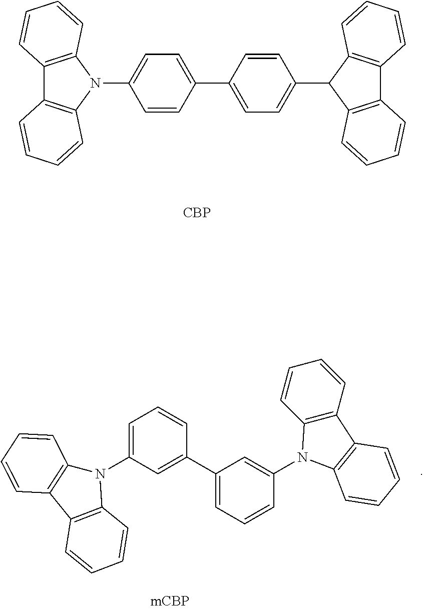 [SMILES: c1ccc2c(c1)-c1ccccc1C2c1ccc(-c2ccc(-n3c4ccccc4c4ccccc43)cc2)cc1 ; "CBP" ; c1cc(-c2cccc(-n3c4ccccc4c4ccccc43)c2)cc(-n2c3ccccc3c3ccccc32)c1]

In an embodiment, a difference between a triplet energy level (eV) of the fourth compound and a singlet energy level (eV) of the fourth compound may be in a range of about 0 eV to about 0.5 eV. For example, a difference between a triplet energy level (eV) of the fourth compound and a singlet energy level (eV) of the fourth compound may be in a range of about 0 eV to about 0.3 eV.

In an embodiment, the fourth compound may include at least one cyclic group including boron (B) and nitrogen (N) as ring-forming atoms.

In an embodiment, the fourth compound may be a C₈-C₆₀ polycyclic group-containing compound including at least two condensed cyclic groups that share a boron atom (B).

In an embodiment, the fourth compound may include a condensed ring in which at least one third ring may be condensed with at least one fourth ring,
the third ring may be a cyclopentane group, a cyclohexane group, a cycloheptane group, a cyclooctane group, a cyclopentene group, a cyclohexene group, a cycloheptene group, a cyclooctene group, an adamantane group, a norbornene group, a norbornane group, a bicyclo[1.1.1]pentane group, a bicyclo[2.1.1]hexane group, a bicyclo[2.2.2]octane group, a benzene group, a pyridine group, a pyrimidine group, a pyridazine group, a pyrazine group, or a triazine group, and
the fourth ring may be a 1,2-azaborinine group, a 1,3-azaborinine group, a 1,4-azaborinine group, a 1,2-dihydro-1,2-azaborinine group, a 1,4-oxaborinine group, a 1,4-thiaborinine group, or a 1,4-dihydroborinine group.

In an embodiment, the second compound may include a compound represented by Formula 2:

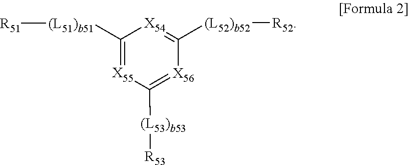

In Formula 2,

L₅₁ to L₅₃ may each independently be a single bond, a C₃-C₆₀ carbocyclic group unsubstituted or substituted with at least one R₁₀ₐ, or a C₁-C₆₀ heterocyclic group unsubstituted or substituted with at least one R₁₀ₐ,
b51 to b53 may each independently be an integer from 1 to 5,
X₅₄ may be N or C(R₅₄), X₅₅ may be N or C(R₅₅), X₅₆ may be N or C(R₅₆), and at least one of X₅₄ to X₅₆ may each be N, and
R₅₁ to R₅₆ and R₁₀ₐ are each respectively the same as described in the specification.

In embodiments, the third compound may include a compound represented by Formula 3-1, a compound represented by Formula 3-2, a compound represented by Formula 3-3, a compound represented by Formula 3-4, a compound represented by Formula 3-5, or any combination thereof:

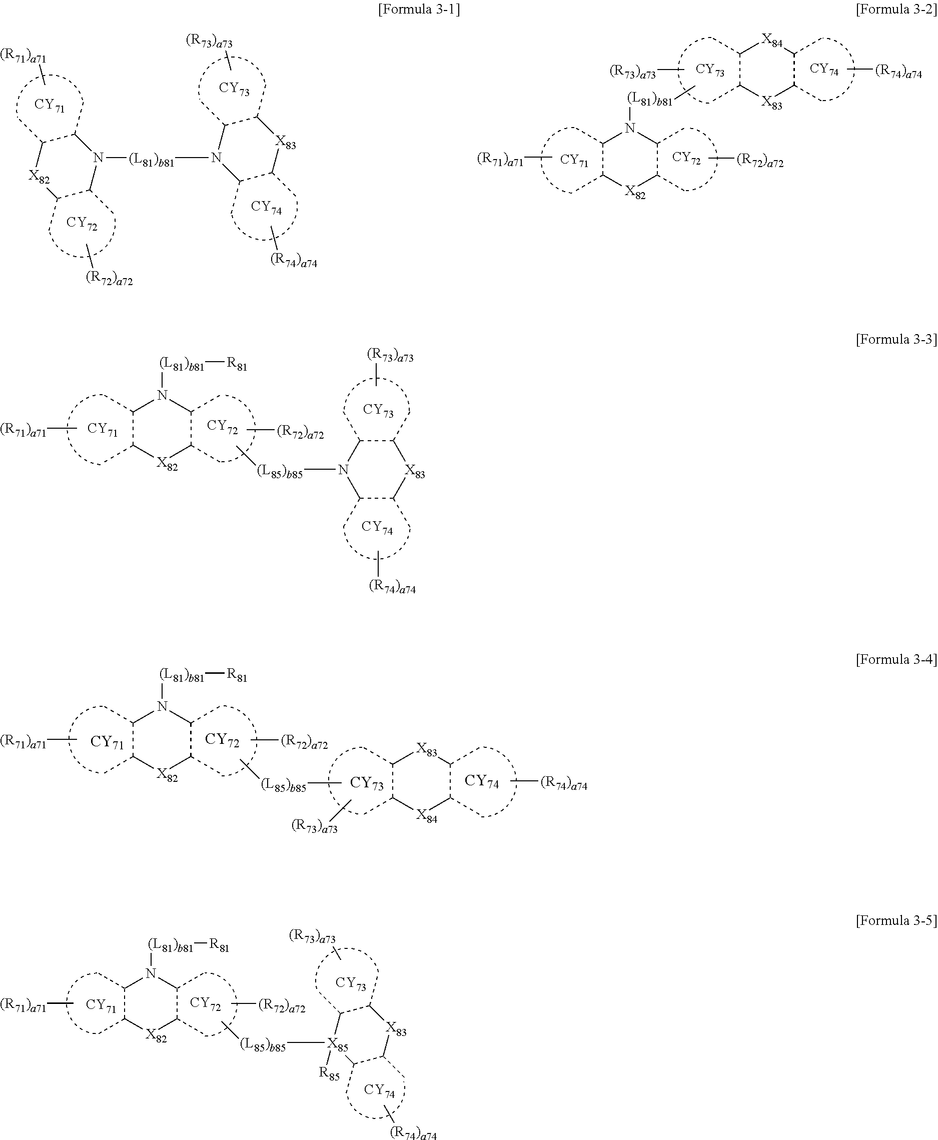

In Formulae 3-1 to 3-5,
ring CY₇₁ to ring CY₇₄ may each independently be a π electron-rich C₃-C₆₀ cyclic group or a pyridine group,
X₈₂ may be a single bond, O, S, N-[(L₈₂)_{b82}-R₈₂], B [(L₈₂)_{b82}-R₈₂], C(R₈₂ₐ) (R_{82b}), or Si(R₈₂ₐ)(R_{82b}),
X₈₃ may be a single bond, O, S, N-[(L₈₃)_{b83}-R₈₃], B [(L₈₃)_{b83}-R₈₃], C(R₈₃ₐ) (R_{83b}), or Si(R₈₃ₐ)(R_{83b}),
X₈₄ may be O, S, N-[(L₈₄)_{b84}-R₈₄], B [(L₈₄)_{b84}-R₈₄], C(R₈₄ₐ)(R_{84b}), or Si(R₈₄ₐ)(R_{84b}),
X₈₅ may be C or Si,
L₈₁ to L₈₅ may each independently be a single bond, *—C(Q₄)(Q₅)-*′, *—Si(Q₄)(Q₅)—*′, a π electron-rich C₃-C₆₀ cyclic group unsubstituted or substituted with at least one R₁₀ₐ, or a pyridine group unsubstituted or substituted with at least one R₁₀ₐ, wherein Q₄ and Q₅ are each independently the same as described in connection with Q₁ as defined herein,
b81 to b85 may each independently be an integer from 1 to 5,
R₇₁ to R₇₄, R₈₁ to R₈₅, R₈₂ₐ, R_{82b}, R₈₃ₐ, R_{83b}, R₈₄ₐ, and R_{84b} are each respectively the same as described herein,
a71 to a74 may each independently be an integer from 0 to 20, and
R₁₀ₐ is the same as described herein.

In embodiments, the fourth compound may be a compound represented by Formula 502, a compound represented by Formula 503, or any combination thereof:

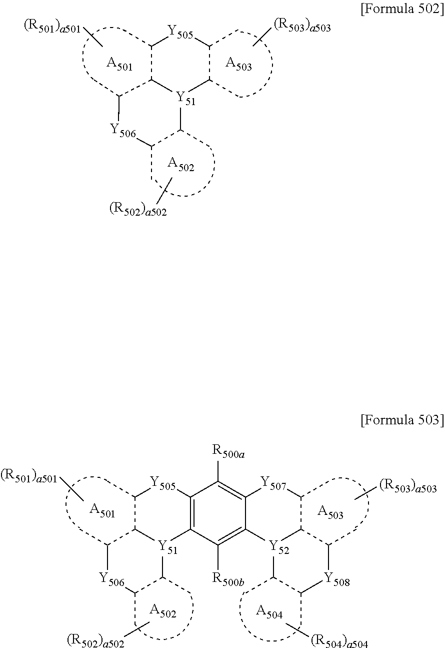

In Formulae 502 and 503,
ring A₅₀₁ to ring A₅₀₄ may each independently be a C₃-C₆₀ carbocyclic group or a C₁-C₆₀ heterocyclic group,
Y₅₀₅ may be O, S, N(R₅₀₅), B(R₅₀₅), C(R₅₀₅ₐ) (R_{505b}), or Si(R₅₀₅ₐ)(R_{505b}),
Y₅₀₆ may be O, S, N(R₅₀₆), B(R₅₀₆), C(R₅₀₆ₐ)(R_{506b}), or Si(R₅₀₆ₐ)(R_{506b}),
Y₅₀₇ may be O, S, N(R₅₀₇), B(R₅₀₇), C(R₅₀₇ₐ)(R_{507b}), or Si(R₅₀₇ₐ) (R_{507b}),
Y₅₀₈ may be O, S, N(R₅₀₈), B(R₅₀₈), C(R₅₀₈ₐ)(R_{508b}), or Si(R₅₀₈ₐ)(R_{508b}),
Y₅₁ and Y₅₂ may each independently be B, P(═O), or S(═O),
R₅₀₀ₐ, R_{500b}, R₅₀₁ to R₅₀₈, R₅₀₅ₐ, R_{505b}, R₅₀₆ₐ, R_{506b}, R₅₀₇ₐ, R_{507b}, R₅₀₈ₐ, and R_{508b} are respectively the same as described in the specification, and
a501 to a504 may each independently be an integer from 0 to 20.

In an embodiment, the light-emitting device may satisfy at least one of Conditions 1 to 4:

### [Condition 1]

Lowest unoccupied molecular orbital (LUMO) energy level (eV) of the third compound>LUMO energy level (eV) of the fifth compound;

### [Condition 2]

LUMO energy level (eV) of the fifth compound>LUMO energy level (eV) of the second compound;

### [Condition 3]

Highest occupied molecular orbital (HOMO) energy level (eV) of the fifth compound>HOMO energy level (eV) of the third compound;

### [Condition 4]

HOMO energy level (eV) of the third compound>HOMO energy level (eV) of the second compound.

A HOMO energy level and a LUMO energy level of the fifth compound, the second compound, and the third compound may each be a negative value. The HOMO energy levels and the LUMO energy levels may each be measured according to a method of a related art.

In embodiments, an absolute value of a difference between the LUMO energy level of the fifth compound and the LUMO energy level of the second compound may be in a range of about 0.1 eV to about 1.0 eV; an absolute value of a difference between the LUMO energy level of the fifth compound and the LUMO energy level of the third compound may be in a range of about 0.1 eV to about 1.0 eV; an absolute value of a difference between a HOMO energy level of the fifth compound and a HOMO energy level of the second compound may be equal to or less than 1.25 eV (for example, be in a range of about 0.2 eV to about 1.25 eV); or an absolute value of a difference between the HOMO energy level of the fifth compound and the HOMO energy level of the third compound may be equal to or less than 1.25 eV (for example, in a range of about 0.2 eV to about 1.25 eV).

When the relationships between LUMO energy level and HOMO energy level satisfy the conditions as described above, a balance between holes and electrons injected into the emission layer can be achieved.

In an embodiment, the fifth compound, which is a transition metal-containing organometallic compound, may include a platinum atom and a tetradentate ligand bonded to the platinum atom, and one of bonds between the platinum atom and the tetradentate ligand may be a platinum-carbene bond.

In an embodiment, the fifth compound, which is a transition metal-containing organometallic compound, may include an iridium atom and a tetradentate ligand bonded to the iridium atom, and one of bonds between the iridium atom and the tetradentate ligand may be an iridium-carbene bond.

In another embodiment, the fifth compound may be represented by Formula 602:

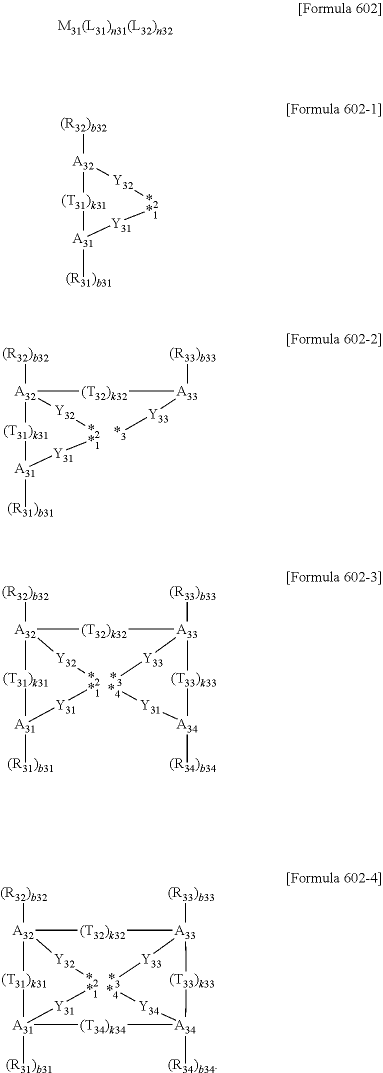

In Formulae 602 and 602-1 to 602-4,
M₃₁ may be a first-row transition metal of the Periodic Table of Elements, a second-row transition metal of the Periodic Table of Elements, or a third-row transition metal of the Periodic Table of Elements,
L₃₁ may be a ligand represented by one of Formulae 602-1 to 602-4,
L₃₂ may be a monodentate ligand, a bidentate ligand, or a tridentate ligand,
n31 may be 1 or 2,
n32 may be 0, 1, 2, 3, or 4,
A₃₁ to A₃₄ may each independently be a C₅-C₃₀ carbocyclic group or a C₁-C₃₀ heterocyclic group,
T₃₁ to T₃₄ may each independently be a single bond, a double bond, *—O—*′, *—S—*′, *—C(═O)—*′, *—S(═O)—*′, *—C(R₃₅)(R₃₆)—*′, *—C(R₃₅)—C(R₃₆)—*′, *—C(R₃₅)═*′, *—Si(R₃₅)(R₃₆)—*′, *—B(R₃₅)—*′, *—N(R₃₅)—*′, or *—P(R₃₅)—*′,
k31 to k34 may each independently be 1, 2, or 3,
Y₃₁ to Y₃₄ may each independently be a single bond, *—O—*′, *—S—*′, *—C(R₃₇)(R₃₈)—*′, *—Si(R₃₇)(R₃₈)—*′, *—B(R₃₇)—*′, *—N(R₃₇)—*′, or *—P(R₃₇)—*′,
*1, *2, *3, and *4 each indicate a binding site to M₃₁,
R₃₁ to R₃₈ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₅-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
R₃₁ to R₃₈ may optionally be bonded to each other to form a C₅-C₆₀ carbocyclic group unsubstituted or substituted with at least one R₁₀ₐ or a C₁-C₆₀ heterocyclic group unsubstituted or substituted with at least one R₁₀ₐ,
b31 to b34 may each independently be an integer from 0 to 10,
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

In an embodiment, the emission layer may include a hole transporting compound, an electron transporting compound, a transition metal-containing organometallic compound, and a delayed fluorescence compound.

In an embodiment, the delayed fluorescence compound may include at least one donor group and at least one acceptor group.

In an embodiment, the delayed fluorescence compound may satisfy Equation 1:

$$[ Equation 1 ]$$

$$Δ {E}_{ST} = S 1 ( delayed fluorescence ) - T 1 ( delayed fluorescence ) \leq 0.3 eV$$

In Equation 1, S1 (delayed fluorescence) is a lowest excitation singlet energy level (eV) of the delayed fluorescence compound, and T1 (delayed fluorescence) is a lowest excitation triplet energy level (eV) of the delayed fluorescence compound.

In an embodiment, the emission layer may include a host, an emitter, a sensitizer, or any combination thereof.

In an embodiment, the emission layer may not include an anthracene-based compound.

In an embodiment, the light-emitting device may further include a capping layer outside the first electrode and/or outside the second electrode.

In an embodiment, the light-emitting device may further include at least one of a first capping layer located outside the first electrode and a second capping layer located outside the second electrode, and at least one of the first capping layer and the second capping layer may each independently include an amine-based compound represented by Formula 1. The first capping layer and/or the second capping layer are each the same as described herein.

In an embodiment, the light-emitting device may further include:
a first capping layer outside the first electrode and including an amine-based compound represented by Formula 1;
a second capping layer outside the second electrode and including an amine-based compound represented by Formula 1; or
the first capping layer and the second capping layer.

The expression “(interlayer and/or capping layer) includes the amine-based compound represented by Formula 1” as used herein may be understood as “(interlayer and/or capping layer) may include one kind of amine-based compound represented by Formula 1 or two different kinds of amine-based compounds, each independently represented by Formula 1.”

In an embodiment, the interlayer and/or the capping layer may include Compound 1 only as the amine-based compound. In this regard, Compound 1 may be present in the emission layer of the light-emitting device. In an embodiment, the interlayer may include, as the amine-based compound, Compound 1 and Compound 2. In this regard, Compound 1 and Compound 2 may be present in the same layer (for example, both Compound 1 and Compound 2 may be present in the emission layer), or may be present in different layers (for example, Compound 1 may be present in the emission layer, and Compound 2 may be present in the electron transport region).

The term “interlayer” as used herein may be a single layer and/or all of layers between the first electrode and the second electrode of the light-emitting device.

Another aspect of the embodiments provides an electronic apparatus including the light-emitting device. The electronic apparatus may further include a thin-film transistor. For example, the electronic apparatus may further include a thin-film transistor including a source electrode and a drain electrode, wherein the first electrode of the light-emitting device may be electrically connected to the source electrode or the drain electrode. In an embodiment, the electronic apparatus may further include a color filter, a color conversion layer, a touch screen layer, a polarizing layer, or any combination thereof. Further details on the electronic apparatus may be the same as provided herein.

Another embodiment provides an electronic device which may include the light-emitting device.

For example, the electronic device may be a flat panel display, a curved display, a computer monitor, a medical monitor, a television (TV), an advertisement board, an indoor light, an outdoor light, a signal light, a head-up display, a fully transparent display, a partially transparent display, a flexible display, a rollable display, a foldable display, a stretchable display, a laser printer, a telephone, a portable phone, a tablet personal computer, a phablet, a personal digital assistant (PDA), a wearable device, a laptop computer, a digital camera, a camcorder, a viewfinder, a microdisplay, a three-dimensional (3D) display, a virtual reality display, an augmented reality display, a vehicle, a video wall with multiple displays tiled together, a theater screen, a stadium screen, a phototherapy device, or a sign.

An embodiment provides an amine-based compound which may be represented by Formula 1. Details on Formula 1 are the same as described herein.

Synthesis methods of the amine-based compound may be recognizable by one of ordinary skill in the art by referring to Synthesis Examples and/or Examples provided below.

### [Description of Formula 1]

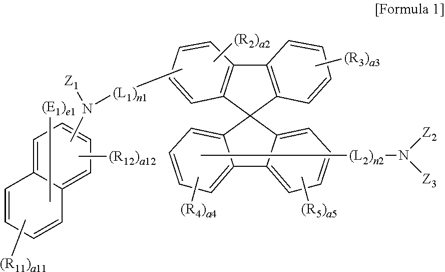

In Formula 1, L₁ and L₂ may each independently be a single bond, a C₅-C₆₀ carbocyclic group unsubstituted or substituted with at least one R₁₀ₐ, or a C₁-C₆₀ heterocyclic group unsubstituted or substituted with at least one R₁₀ₐ.

In an embodiment, L₁ and L₂ may each independently be:
a single bond; or
a benzene group, a naphthalene group, a fluorene group, a carbazole group, a dibenzofuran group, or a dibenzothiophene group, each unsubstituted or substituted with at least one R₁₀ₐ.

In another embodiment, L₁ and L₂ may each independently be a single bond.

In Formula 1, n1 and n2 may each independently be an integer from 1 to 3.

In an embodiment, n1 and n2 may each independently be 1 or 2.

In an embodiment, n1 and n2 may each be 1.

In Formula 1, Z₁ to Z₃ and E₁ may each independently be deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂).

In an embodiment, Z₁ to Z₃ and E₁ may each independently be a benzene group, a naphthalene group, a fluorene group, a carbazole group, a dibenzofuran group, a dibenzothiophene group, an azacarbazole group, an azadibenzofuran group, an azadibenzothiophene group, a pyridine group, a pyrimidine group, a pyrazine group, a quinoline group, or a quinoxaline group, each unsubstituted or substituted with at least one R₁₀ₐ.

In embodiments, Z₁ to Z₃ and E₁ may each independently be a group represented by any one of Formulae 1A-1 to 1A-16:

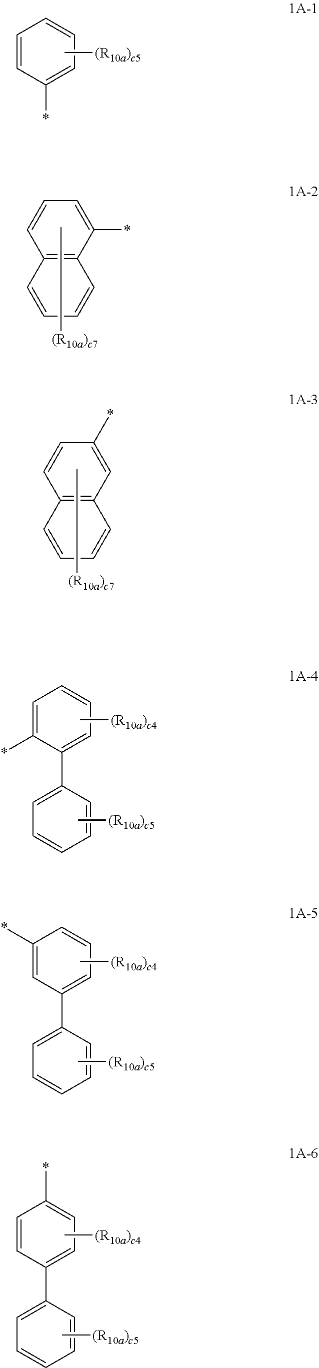

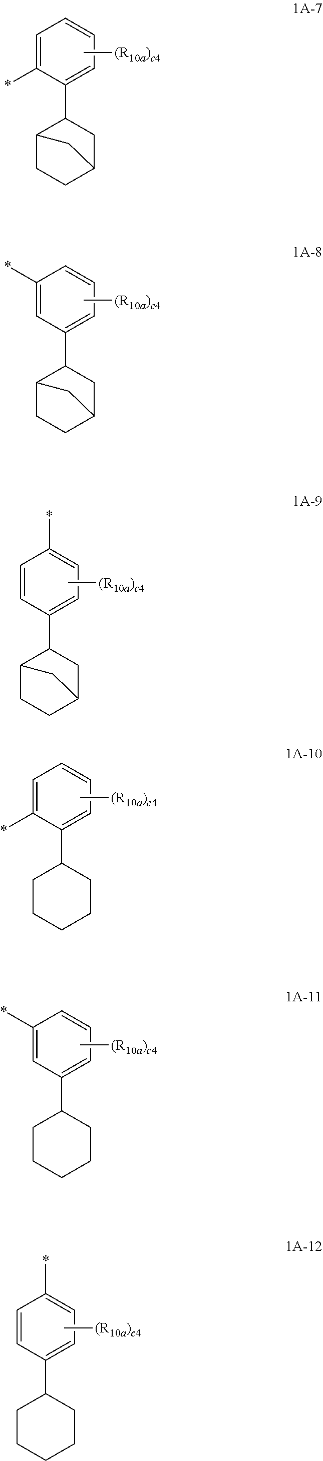

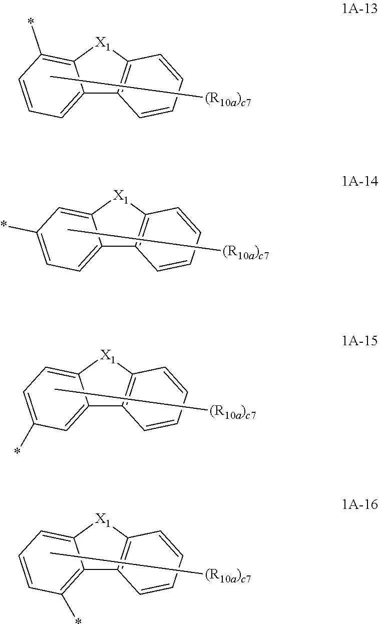

In Formulae 1A-1 to 1A-16,
R₁₀ₐ is the same as described herein,
c4 may be an integer from 0 to 4,
c5 may be an integer from 0 to 5,
c7 may be an integer from 0 to 7,
X₁ may be N(R₁), O, or S,
R₁ is the same as described in connection with R₁₁, and
* indicates a binding site to N or a naphthalene group in Formula 1.

In Formula 1, e1 may be an integer from 1 to 7.

In an embodiment, Z₂ and Z₃ may be identical to or different from each other.

In another embodiment, Z₂ and Z₃ may be different from each other.

In Formula 1, R₁₁, R₁₂, and R₂ to R₅ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂).

In Formula 1, R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₅-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₅-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a C₃-C₆₀ carbocyclic group, a C₁-C₆₀ heterocyclic group, or any combination thereof.

In an embodiment, R₁₁, R₁₂, and R₂ to R₅ may each independently be:
hydrogen, deuterium, —F, —Cl, —Br, —I, a hydroxyl group, a cyano group, a nitro group, a C₁-C₂₀ alkyl group, or a C₁-C₂₀ alkoxy group;
a C₁-C₂₀ alkyl group or a C₁-C₂₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a naphthyl group, a pyridinyl group, a pyrimidinyl group, or any combination thereof;
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a terphenyl, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzothiazolyl group, a benzoisoxazolyl group, a benzoisoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, an azafluorenyl group, or an azadibenzosilolyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a terphenyl, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzoisothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, —O(Q₃₁), —S(Q₃₁), —Si(Q₃₁)(Q₃₂)(Q₃₃), —N(Q₃₁)(Q₃₂), —B(Q₃₁)(Q₃₂), —P(Q₃₁)(Q₃₂), —C(═O)(Q₃₁), —S(═O)₂(Q₃₁), —P(═O)(Q₃₁)(Q₃₂), or any combination thereof; or
—C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂), and
Q₁ to Q₃ and Q₃₁ to Q₃₃ are each independently the same as described in the specification.

In another embodiment, R₁₁, R₁₂, and R₂ to R₅ may each independently be: hydrogen, deuterium, —F, or a cyano group;
a C₁-C₂₀ alkyl group unsubstituted or substituted with deuterium, —F, a cyano group, a phenyl group, a deuterated phenyl group, a fluorinated phenyl group, a (C₁-C₂₀ alkyl)phenyl group, a biphenyl group, a deuterated biphenyl group, a fluorinated biphenyl group, a (C₁-C₂₀ alkyl) biphenyl group, or any combination thereof;
a C₃-C₁₀ cycloalkyl group, a phenyl group, a naphthyl group, a carbazolyl group, a dibenzofuranyl group, or a dibenzothiophenyl group, each unsubstituted or substituted with deuterium, —F, a cyano group, a C₁-C₂₀ alkyl group, a deuterated C₁-C₂₀ alkyl group, a fluorinated C₁-C₂₀ alkyl group, a C₃-C₁₀ cycloalkyl group, a deuterated C₃-C₁₀ cycloalkyl group, a fluorinated C₅-C₁₀ cycloalkyl group, a phenyl group, a deuterated phenyl group, a fluorinated phenyl group, a (C₁-C₂₀ alkyl)phenyl group, a biphenyl group, a deuterated biphenyl group, a fluorinated biphenyl group, a (C₁-C₂₀ alkyl) biphenyl group, or any combination thereof; or
—Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂), and
Q₁ to Q₃ may each independently be a C₃-C₁₀ cycloalkyl group, a phenyl group, a naphthyl group, a carbazolyl group, a dibenzofuranyl group, or a dibenzothiophenyl group, each unsubstituted or substituted with deuterium, —F, a cyano group, a C₁-C₂₀ alkyl group, a deuterated C₁-C₂₀ alkyl group, a fluorinated C₁-C₂₀ alkyl group, a C₃-C₁₀ cycloalkyl group, a deuterated C₃-C₁₀ cycloalkyl group, a fluorinated C₃-C₁₀ cycloalkyl group, a phenyl group, a deuterated phenyl group, a fluorinated phenyl group, a (C₁-C₂₀ alkyl)phenyl group, a biphenyl group, a deuterated biphenyl group, a fluorinated biphenyl group, a (C₁-C₂₀ alkyl) biphenyl group, or any combination thereof.

In Formula 1,
a11 may be an integer from 0 to 4,
a12 may be an integer from 0 to 3,
a2 may be an integer from 0 to 3, and
a3 to a5 may each independently be an integer from 0 to 4.

In an embodiment, in Formula 1, a moiety represented by

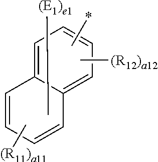

may be a moiety represented by one of Formulae 1B-1 to 1B-14:

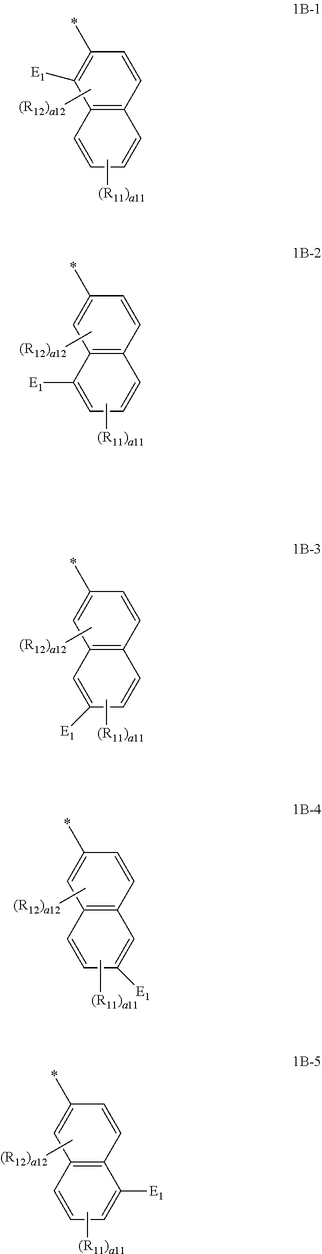

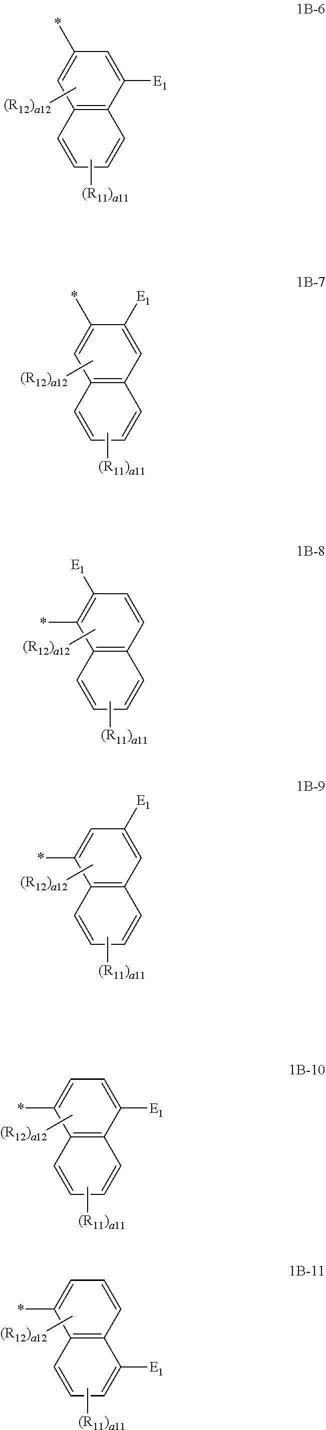

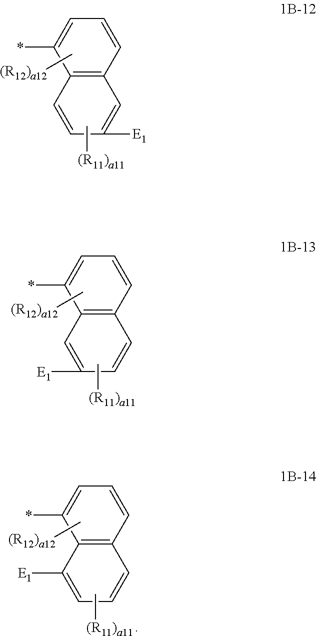

In Formulae 1B-1 to 1B-14,
E₁, R₁₁, R₁₂, a11, and a12 may respectively be the same as described above, and
* indicates a binding site to a neighboring N.

In an embodiment, the amine-based compound may be represented by Formula 1-1:

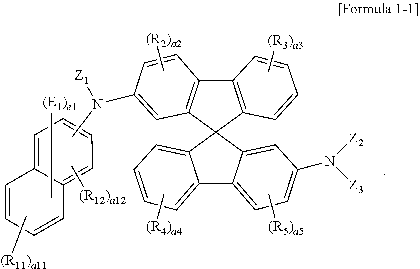

In Formula 1-1,
a5 may be an integer from 0 to 3, and
Z₁ to Z₃, E₁, e1, R₁₁, R₁₂, R₂ to R₅, a11, a12, and a2 to a4 may be the same as described herein.

All descriptions regarding Formula 1 may be applied to Formula 1-1 as well.

As an amine-based compound represented by Formula 1 according to an embodiment essentially introduces a substituent to a naphthyl group, the color coordinates may be improved to emit deep blue light, and the structural stability of the compound may be secured.

The hole transport region, for example, at least one of the hole transport layer and the hole injection layer may include an amine-based compound represented by Formula 1. Accordingly, an amine-based compound represented by Formula 1 may be used as a hole transport material.

When the amine-based compound represented by Formula 1 is applied to a light-emitting device, luminescence efficiency and device lifespan may be improved.

In addition, by using the amine-based compound, an electronic device (e.g., an organic light-emitting device) having low driving voltage, high efficiency, high color purity, and long lifespan characteristics may be implemented.

### [Description of Formulae 2, 3-1 to 3-5, 502, and 503]

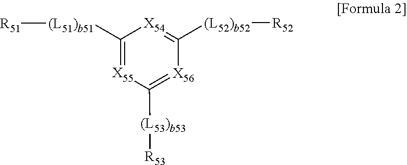

In Formula 2, b51 to b53 may respectively be numbers of L₅₁ to L₅₃, and b51 to b53 may each independently be an integer from 1 to 5.

When b51 is 2 or more, two or more of L₅₁(s) may be identical to or different from each other, when b52 is 2 or more, two or more of L₅₂(s) may be identical to or different from each other, and when b53 is 2 or more, two or more of L₅₃(s) may be identical to or different from each other. In an embodiment, b51 to b53 may each independently be 1 or 2.

In Formula 2, L₅₁ to L₅₃ may each independently be:
a single bond; or
a benzene group, a naphthalene group, an anthracene group, a phenanthrene group, a triphenylene group, a pyrene group, a chrysene group, a cyclopentadiene group, a furan group, a thiophene group, a silole group, an indene group, a fluorene group, an indole group, a carbazole group, a benzofuran group, a dibenzofuran group, a benzothiophene group, a dibenzothiophene group, a benzosilole group, a dibenzosilole group, an azafluorene group, an azacarbazole group, an azadibenzofuran group, an azadibenzothiophene group, an azadibenzosilole group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isooxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a dibenzooxacilline group, a dibenzothiacilline group, a dibenzodihydroazacilline group, a dibenzodihydrodicilline group, a dibenzodihydrocilline group, a dibenzodioxane group, a dibenzooxathiene group, a dibenzooxazine group, a dibenzopyran group, a dibenzodithiine group, a dibenzothiazine group, a dibenzothiopyran group, a dibenzocyclohexadiene group, a dibenzodihydropyridine group, or a dibenzodihydropyrazine group, each unsubstituted or substituted with deuterium, —F, —Cl, —Br, —I, a hydroxyl group, a cyano group, a nitro group, a C₁-C₂₀ alkyl group, a C₁-C₂₀ alkoxy group, a phenyl group, a naphthyl group, a pyridinyl group, a pyrimidinyl group, a triazinyl group, a fluorenyl group, a dimethylfluorenyl group, a diphenylfluorenyl group, a carbazolyl group, a phenylcarbazolyl group, a dibenzofuranyl group, a dibenzothiophenyl group, a dibenzosilolyl group, a dimethyldibenzosilolyl group, a diphenyldibenzosilolyl group, —O(Q₃₁), —S(Q₃₁), —Si(Q₃₁)(Q₃₂)(Q₃₃), —N(Q₃₁)(Q₃₂), —B(Q₃₁)(Q₃₂), —P(Q₃₁)(Q₃₂), —C(═O)(Q₃₁), —S(═O)₂(Q₃₁), —P(═O)(Q₃₁)(Q₃₂), or any combination thereof, and
Q₃₁ to Q₃₃ may each independently be hydrogen, deuterium, a C₁-C₂₀ alkyl group, a C₁-C₂₀ alkoxy group, a phenyl group, a biphenyl group, a terphenyl group, a pyridinyl group, a pyrimidinyl group, a pyridazinyl group, a pyrazinyl group, or a triazinyl group.

In an embodiment, in Formula 2, a bond between L₅₁ and R₅₁, a bond between L₅₂ and R₅₂, a bond between L₅₃ and R₅₃, a bond between two or more L₅₁(s), a bond between two or more L₅₂(s), a bond between two or more L₅₃(s), a bond between L₅₁ and carbon between X₅₄ and X₅₅, a bond between L₅₂ and carbon between X₅₄ and X₅₆, and a bond between L₅₃ and carbon between X₅₅ and X₅₆ may each be a “carbon-carbon single bond”.

In Formula 2, X₅₄ may be N or C(R₅₄), X₅₅ may be N or C(R₅₅), X₅₆ may be N or C(R₅₆), and at least one of X₅₄ to X₅₆ may each be N. R₅₄ to R₅₆ may be the same as described above. In an embodiment, two or three of X₅₄ to X₅₆ may each be N.

In the specification, R₅₁ to R₅₆, R₇₁ to R₇₄, R₈₁ to R₈₅, R₈₂ₐ, R_{82b}, R₈₃ₐ, R_{83b}, R₈₄ₐ, R_{84b}, R₅₀₀ₐ, R_{500b}, R₅₀₁ to R₅₀₈, R₅₀₅ₐ, R_{505b}, R₅₀₆ₐ, R_{506b}, R₅₀₇ₐ, R_{507b}, R₅₀₈ₐ, and R_{508b} may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂). Q₁ to Q₃ may each be the same as described herein.

For example, R₁ to R₄ and E₁ in Formula 1; R₅₁ to R₅₆, R₇₁ to R₇₄, R₈₁ to R₈₅, R₈₂ₐ, R_{82b}, R₈₃ₐ, R_{83b}, R₈₄ₐ, R_{84b}, R₅₀₀ₐ, R_{500b}, R₅₀₁ to R₅₀₈, R₅₀₅ₐ, R_{505b}, R₅₀₆ₐ, R_{506b}, R₅₀₇ₐ, R_{507b}, R₅₀₈ₐ, and R_{508b} in Formulae 2, 3-1 to 3-5, 502, and 503; and R₁₀ₐ in the specification may each independently be:
hydrogen, deuterium, —F, —Cl, —Br, —I, a hydroxyl group, a cyano group, a nitro group, a C₁-C₂₀ alkyl group, or a C₁-C₂₀ alkoxy group;
a C₁-C₂₀ alkyl group or a C₁-C₂₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a naphthyl group, a pyridinyl group, a pyrimidinyl group, or any combination thereof;
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isooxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzoimidazolyl group, a benzofuranyl group, a benzothiophenyl group, a benzoisothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, an azafluorenyl group, an azadibenzosilolyl group, or a group represented by Formula 91, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isooxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzoimidazolyl group, a benzofuranyl group, a benzothiophenyl group, a benzothiazolyl group, a benzoisoxazolyl group, a benzoisoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, a —O(Q₃₁), a —S(Q₃₁), a —Si(Q₃₁)(Q₃₂)(Q₃₃), a —N(Q₃₁)(Q₃₂), a —B(Q₃₁)(Q₃₂), a —P(Q₃₁)(Q₃₂), a —C(═O)(Q₃₁), a —S(═O)₂(Q₃₁), a —P(═O)(Q₃₁)(Q₃₂), or any combination thereof; or
—C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂), and
Q₁ to Q₃ and Q₃₁ to Q₃₃ may each independently be:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂; or
an n-propyl group, an iso-propyl group, an n-butyl group, an isobutyl group, a sec-butyl group, a tert-butyl group, an n-pentyl group, an isopentyl group, a sec-pentyl group, a tert-pentyl group, a phenyl group, a naphthyl group, a pyridinyl group, a pyrimidinyl group, a pyridazinyl group, a pyrazinyl group, or a triazinyl group, each unsubstituted or substituted with deuterium, a C₁-C₁₀ alkyl group, a phenyl group, a biphenyl group, a pyridinyl group, a pyrimidinyl group, a pyridazinyl group, a pyrazinyl group, a triazinyl group, or any combination thereof:

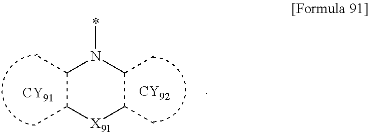

In Formula 91,
ring CY₉₁ and ring CY₉₂ may each independently be a C₅-C₃₀ carbocyclic group unsubstituted or substituted with at least one R₁₀ₐ or a C₁-C₃₀ heterocyclic group unsubstituted or substituted with at least one R₁₀ₐ,
X₉₁ may be a single bond, O, S, N(R₉₁), B(R₉₁), C(R₉₁ₐ)(R_{91b}), or Si(R₉₁ₐ) (R_{91b}),
R₉₁, R₉₁ₐ, and R_{91b} may respectively be the same as described in connection with R₈₂, R₈₂ₐ, and R_{82b},
R₁₀ₐ may be the same as described herein, and
* indicates a binding site to a neighboring atom.

For example, in Formula 91,
ring CY₉₁ and ring CY₉₂ may each independently be a benzene group, a pyridine group, a pyrimidine group, a pyrazine group, a pyridazine group, or a triazine group, each unsubstituted or substituted with at least one R₁₀ₐ, and
R₉₁, R₉₁ₐ, and R_{91b} may each independently be:
hydrogen or a C₁-C₁₀ alkyl group; or
a phenyl group, a pyridinyl group, a pyrimidinyl group, a pyridazinyl group, a pyrazinyl group, or a triazinyl group, each unsubstituted or substituted with deuterium, a C₁-C₁₀ alkyl group, a phenyl group, a biphenyl group, a pyridinyl group, a pyrimidinyl group, a pyridazinyl group, a pyrazinyl group, a triazinyl group, or any combination thereof.

In an embodiment, R₁₁, R₁₂, R₂ to R₅, Z₁ to Z₃, and E₁ in Formula 1; R₅₁ to R₅₆, R₇₁ to R₇₄, R₈₁ to R₈₅, R₈₂ₐ, R_{82b}, R₈₃ₐ, R_{83b}, R₈₄ₐ, R_{84b}, R₅₀₀ₐ, R_{500b}, R₅₀₁ to R₅₀₈, R₅₀₅ₐ, R_{505b}, R₅₀₆ₐ, R_{506b}, R₅₀₇ₐ, R_{507b}, R₅₀₈ₐ, and R_{508b} in Formulae 2, 3-1 to 3-5, 502, 503, and 602; and R₁₀ₐ in the specification may each independently be hydrogen, deuterium, —F, a cyano group, a nitro group, —CH₃, —CD₃, —CD₂H, —CDH₂, —CF₃, —CF₂H, —CFH₂, a group represented by one of Formulae 9-1 to 9-19, a group represented by one of Formulae 10-1 to 10-246, —C(Q₁)(Q₂)(Q₃), —Si(Q₁)(Q₂)(Q₃), or —P(═O)(Q₁)(Q₂) (wherein Q₁ to Q₃ may respectively the same as those described above):

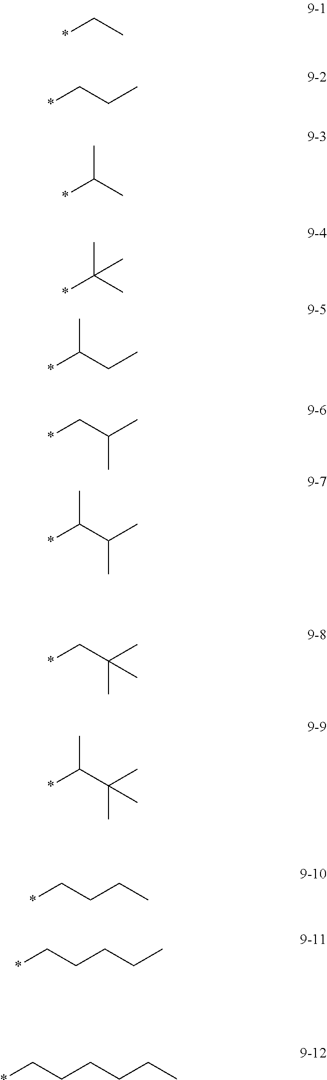

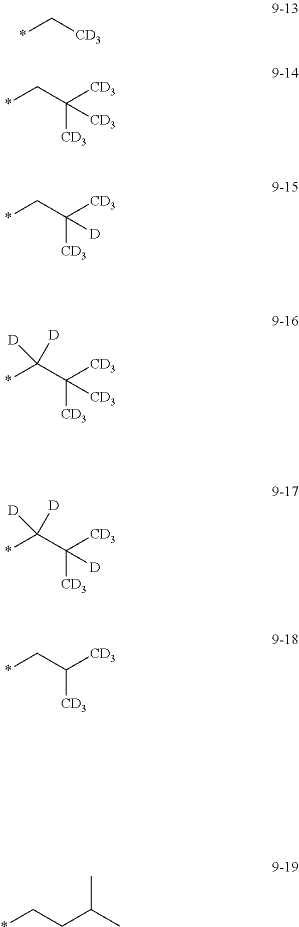

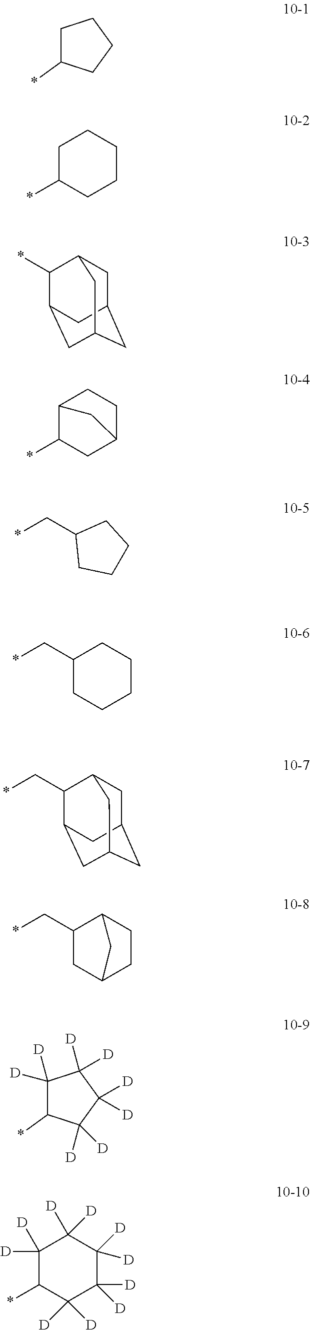

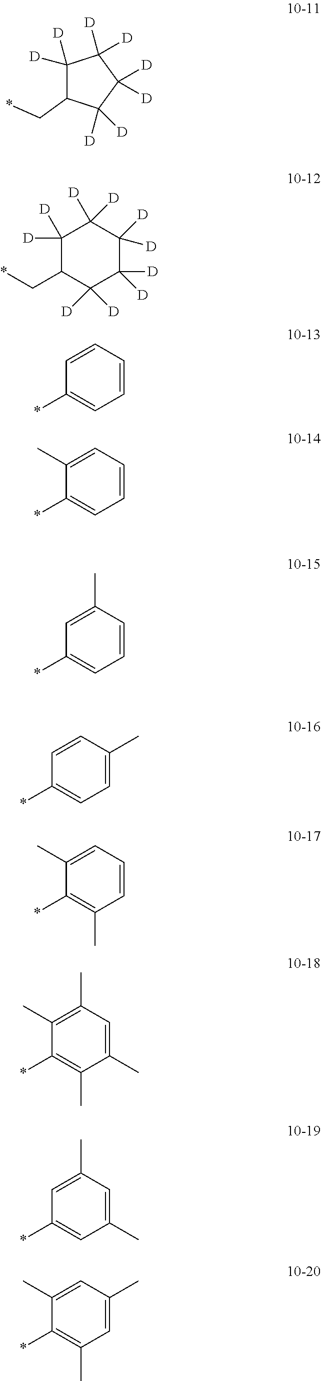

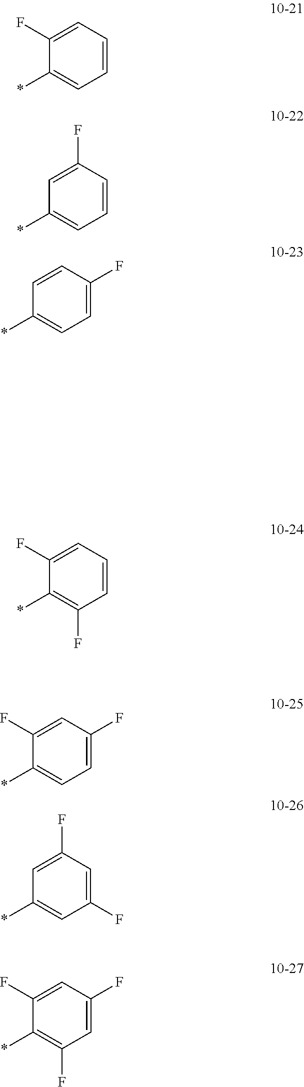

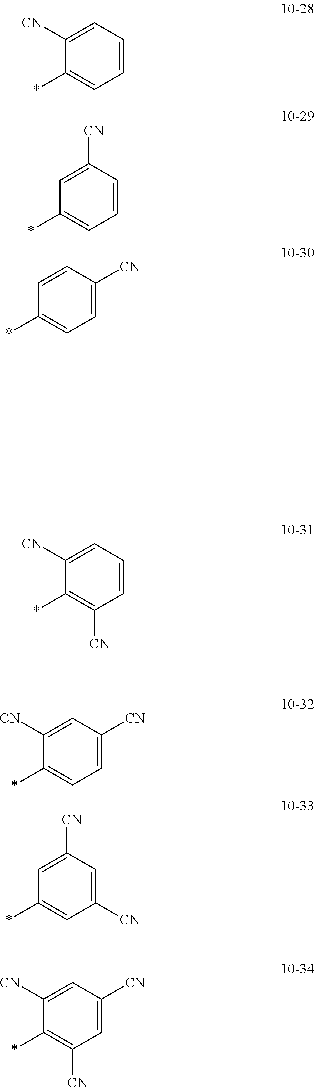

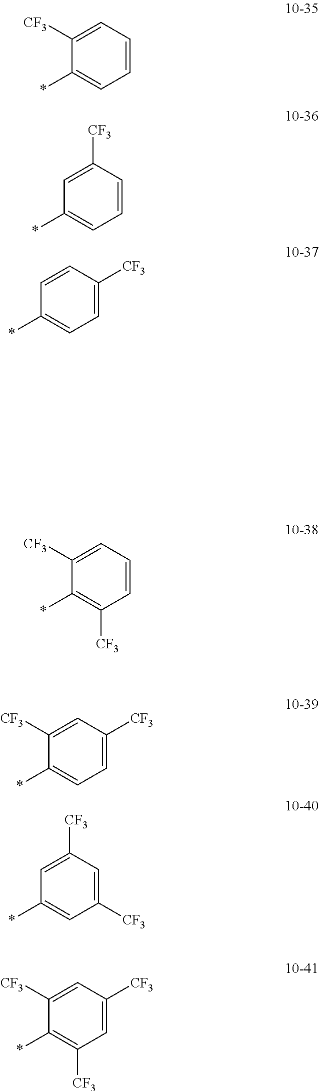

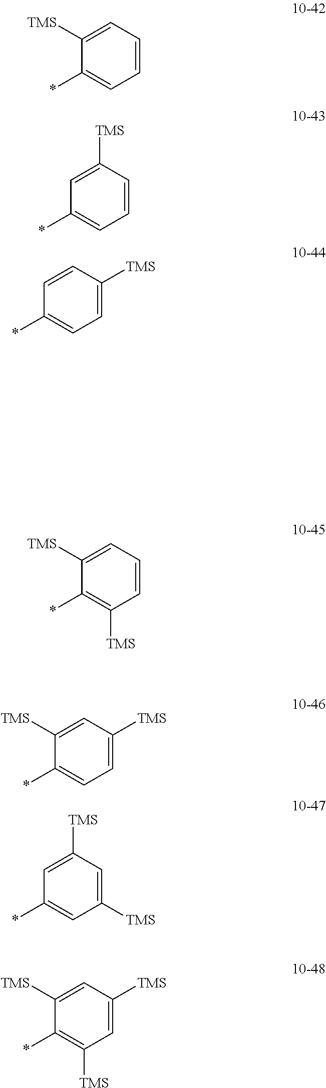

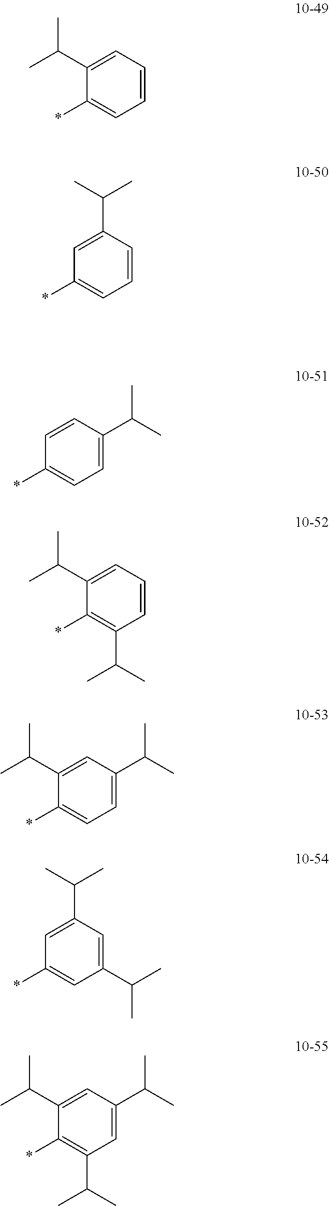

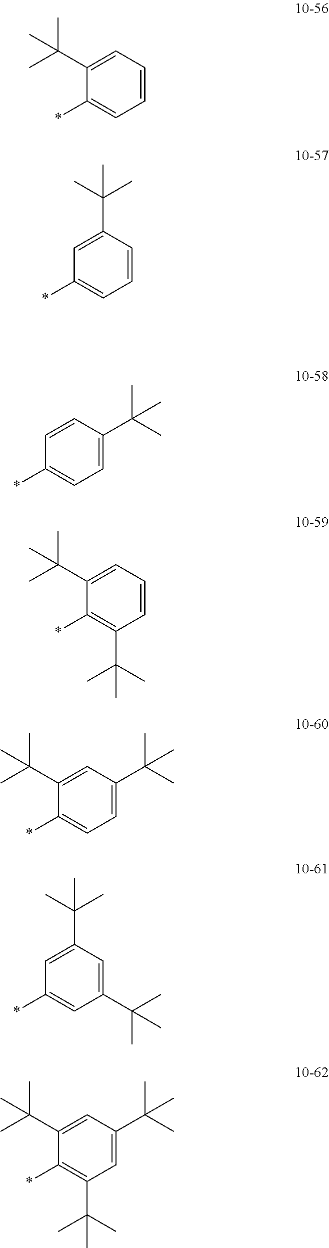

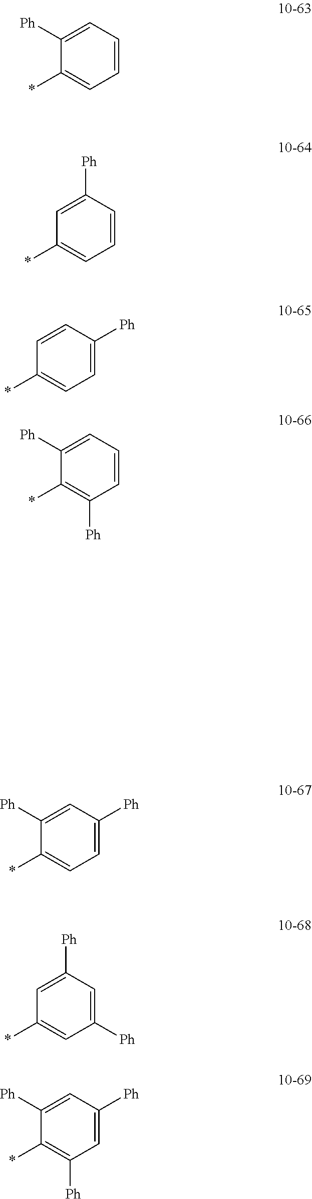

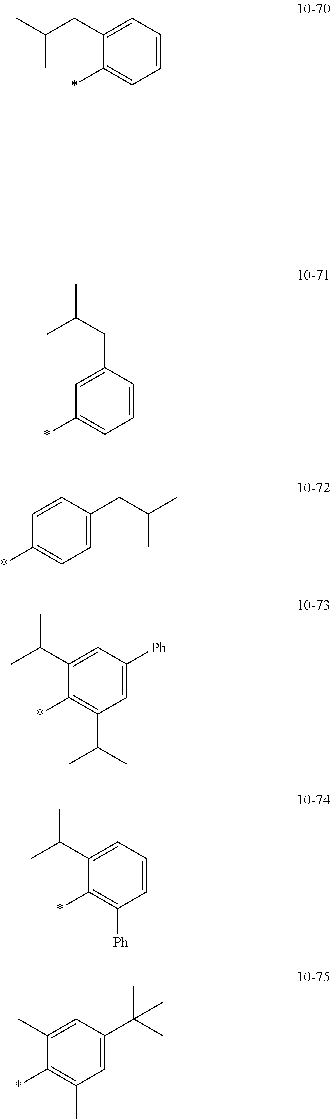

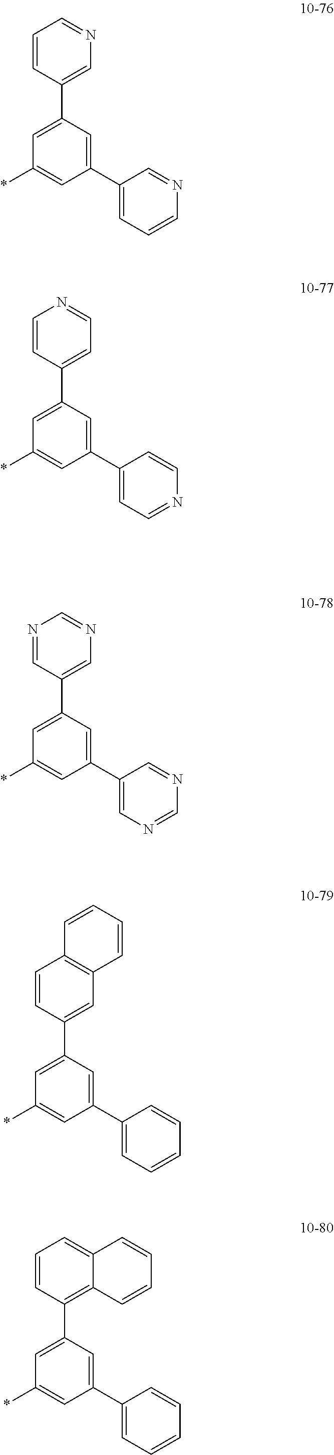 [SMILES: *c1cc(-c2cccnc2)cc(-c2cccnc2)c1 ; *c1cc(-c2ccncc2)cc(-c2ccncc2)c1 ; *c1cc(-c2cncnc2)cc(-c2cncnc2)c1 ; *c1cc(-c2ccccc2)cc(-c2ccc3ccccc3c2)c1 ; *c1cc(-c2ccccc2)cc(-c2cccc3ccccc23)c1]

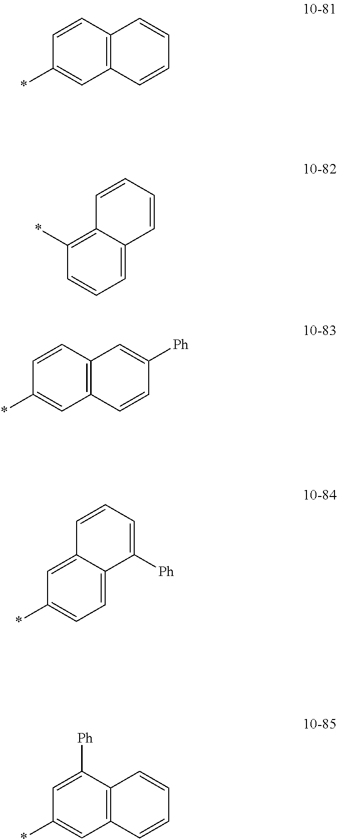 [SMILES: *c1ccc2ccccc2c1 ; *c1cccc2ccccc12 ; *c1ccc2cc(-c3ccccc3)ccc2c1 ; *c1ccc2c(-c3ccccc3)cccc2c1 ; *c1cc(-c2ccccc2)c2ccccc2c1]

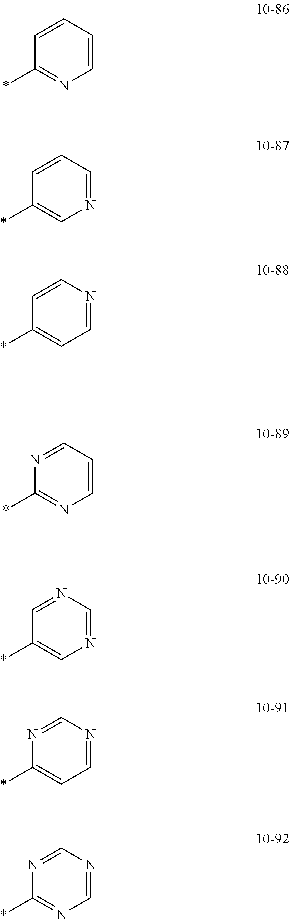

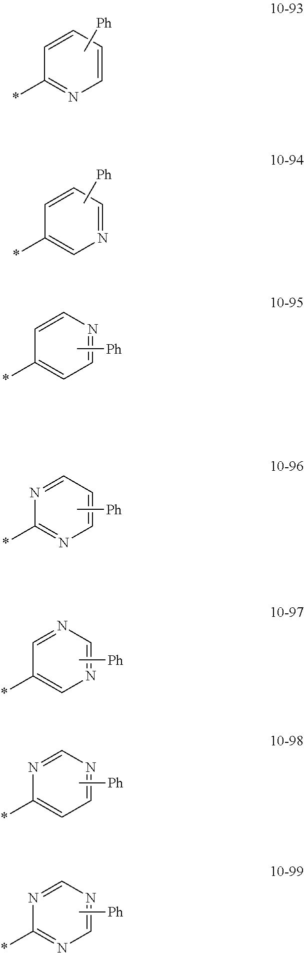

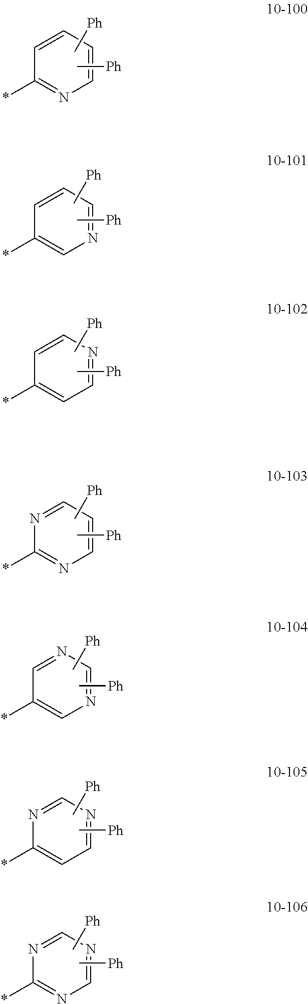

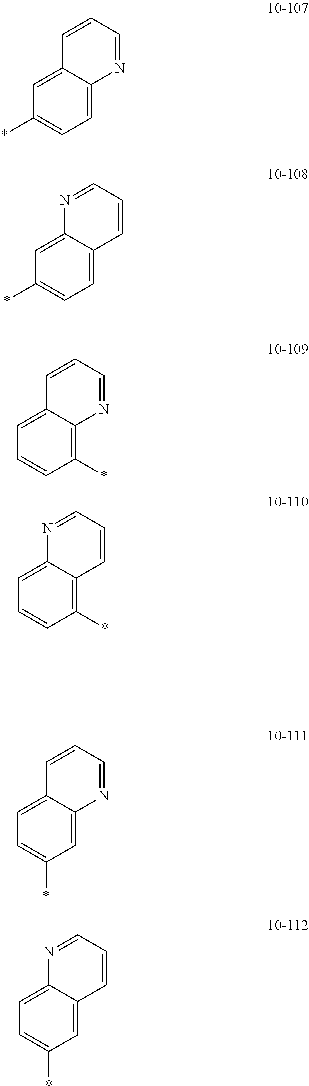

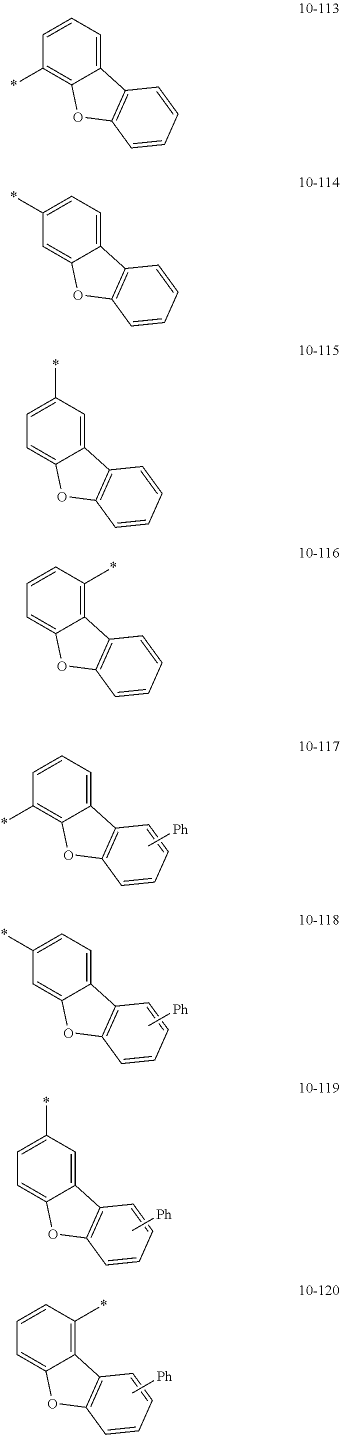

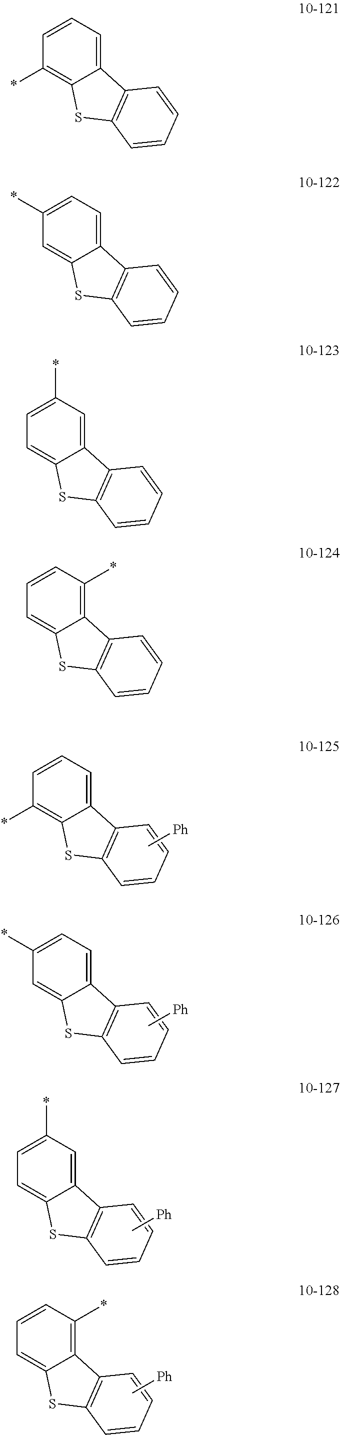

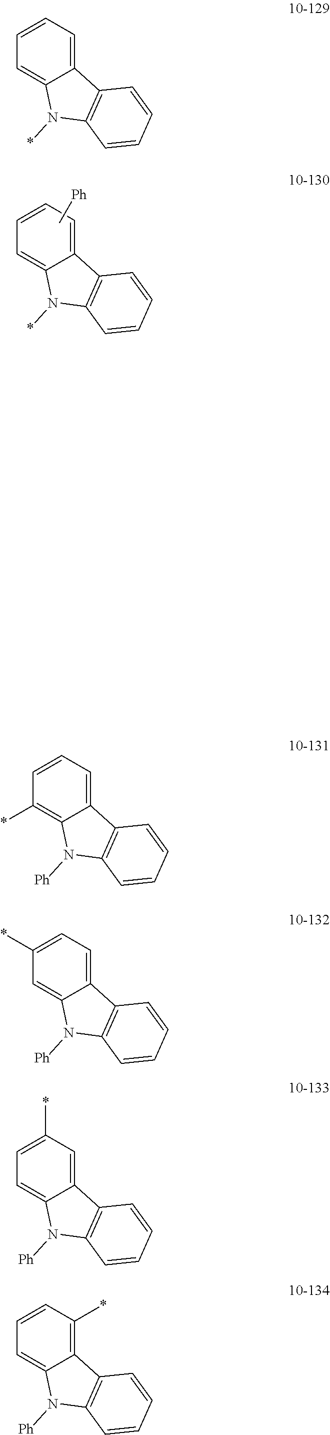

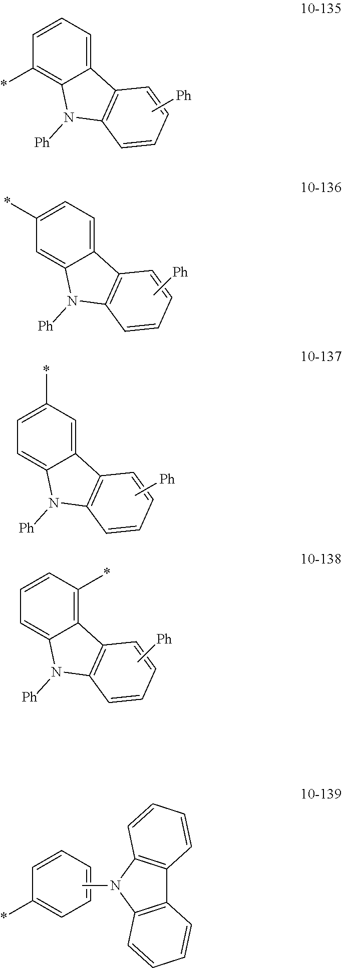

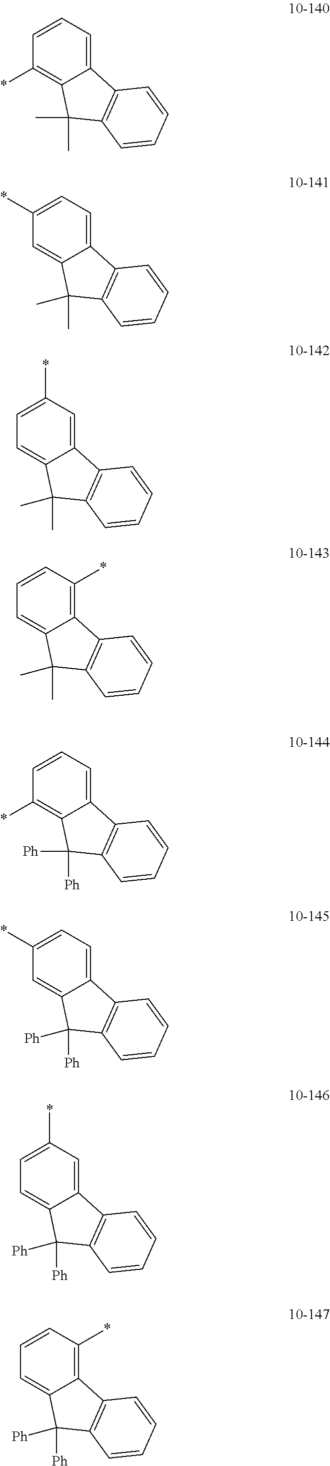

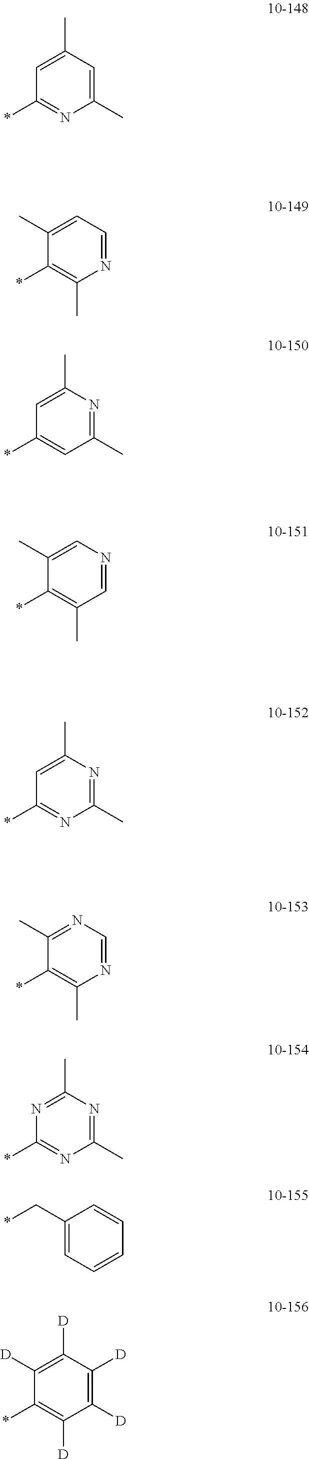

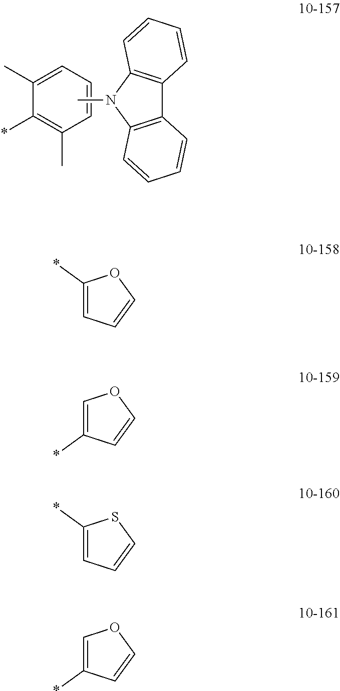

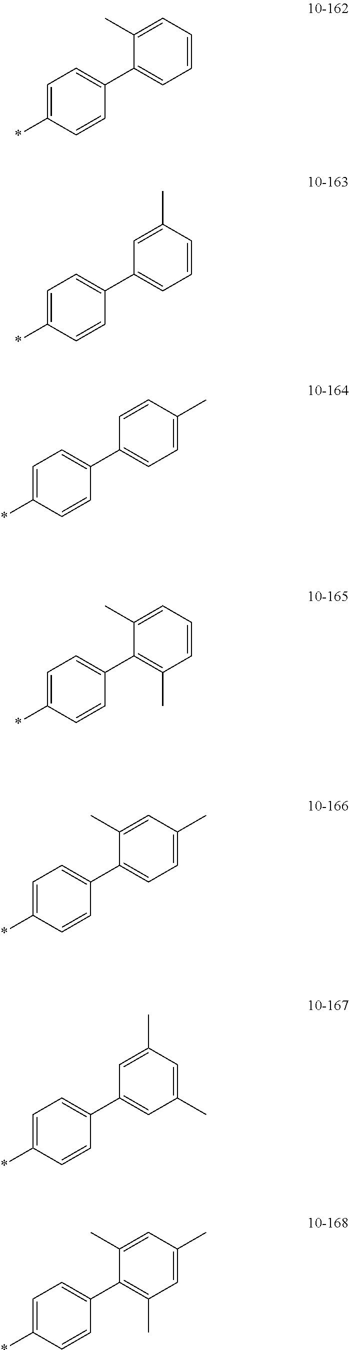

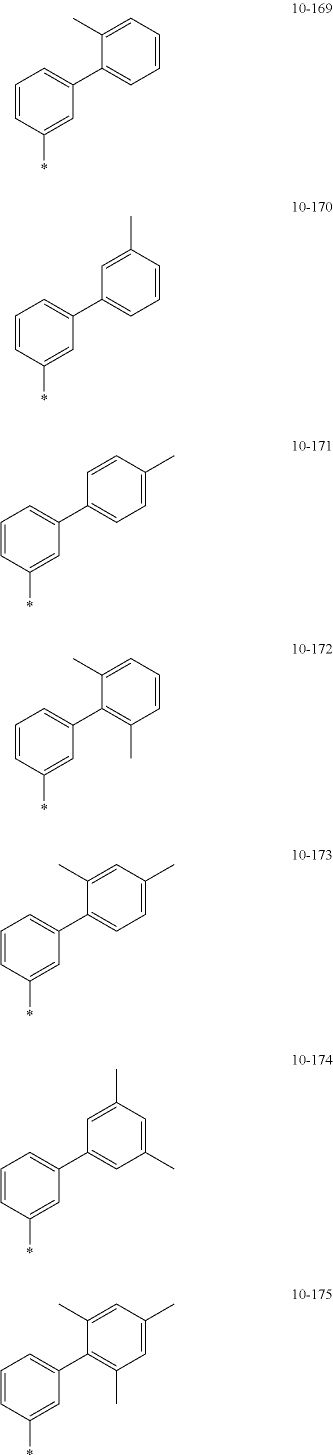

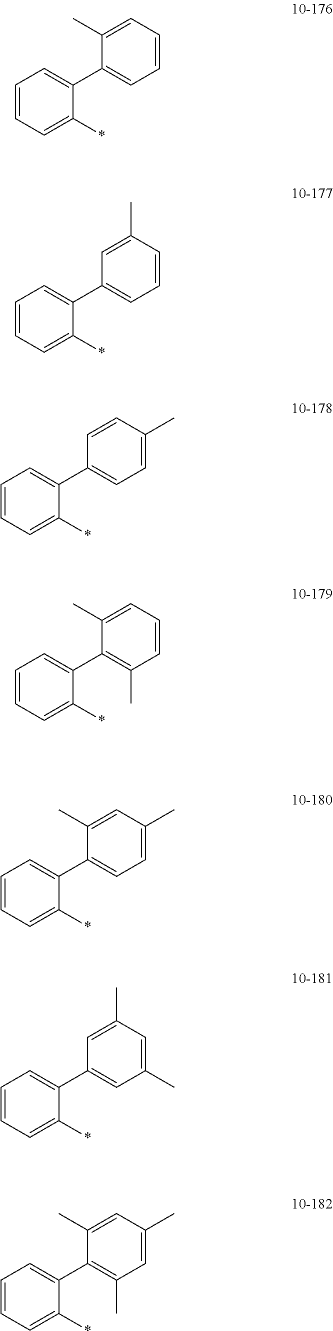

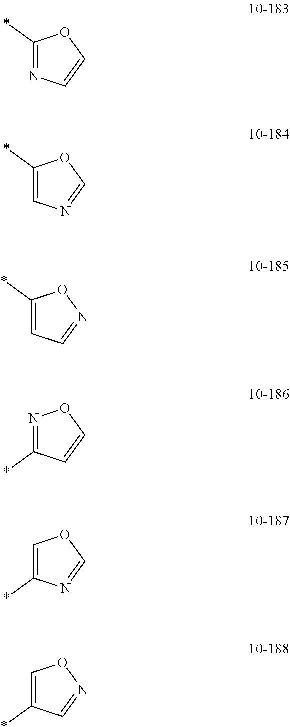 [SMILES: *c1ncco1 ; *c1cnco1 ; *c1ccno1 ; *c1ccon1 ; *c1cocn1 ; *c1cnoc1]

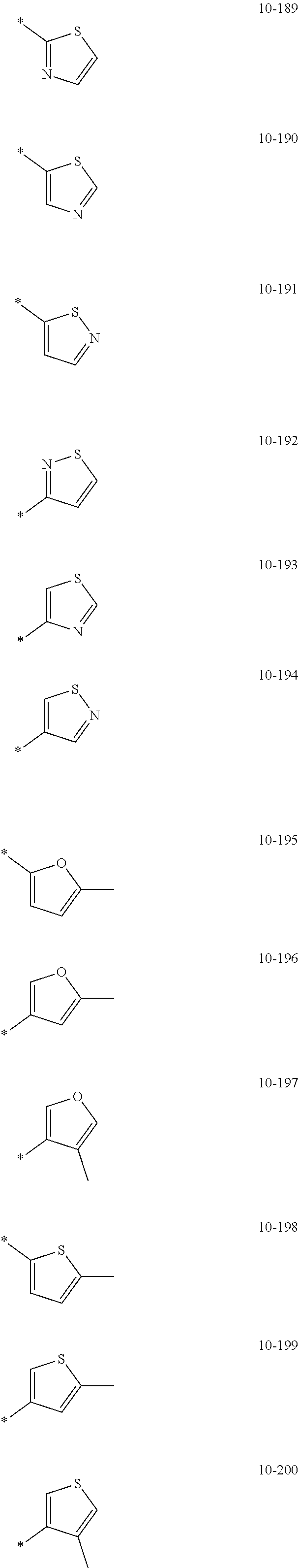

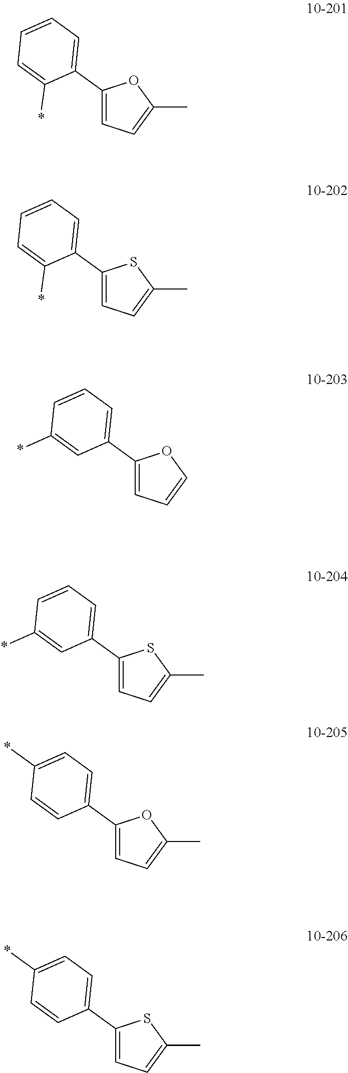 [SMILES: *c1ccccc1-c1ccc(C)o1 ; *c1ccccc1-c1ccc(C)s1 ; *c1cccc(-c2ccco2)c1 ; *c1cccc(-c2ccc(C)s2)c1 ; *c1ccc(-c2ccc(C)o2)cc1 ; *c1ccc(-c2ccc(C)s2)cc1]

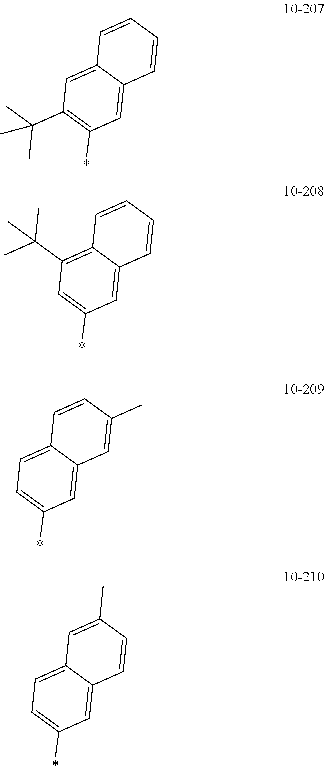 [SMILES: *c1cc2ccccc2cc1C(C)(C)C ; *c1cc(C(C)(C)C)c2ccccc2c1 ; *c1ccc2ccc(C)cc2c1 ; *c1ccc2cc(C)ccc2c1]

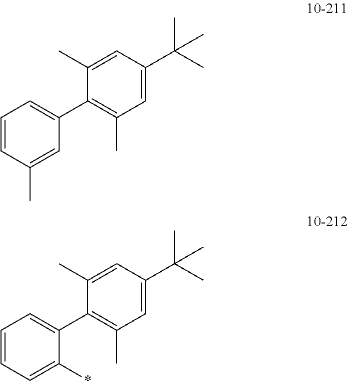

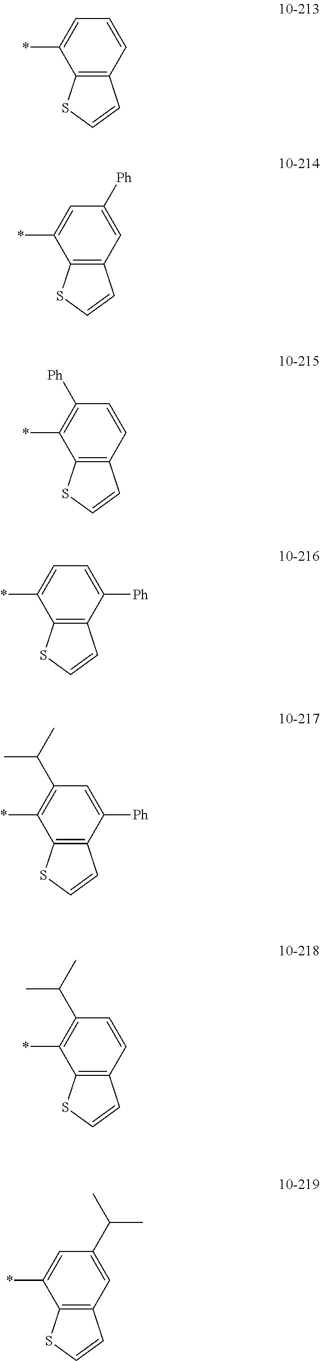

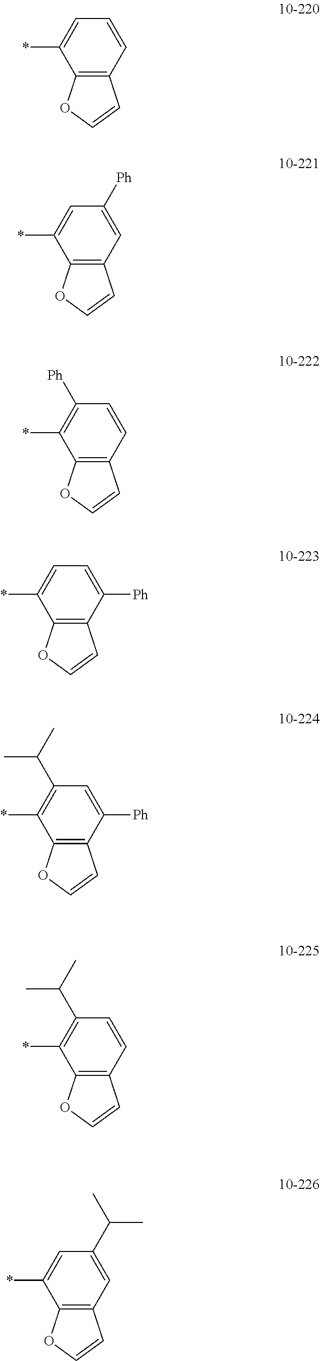

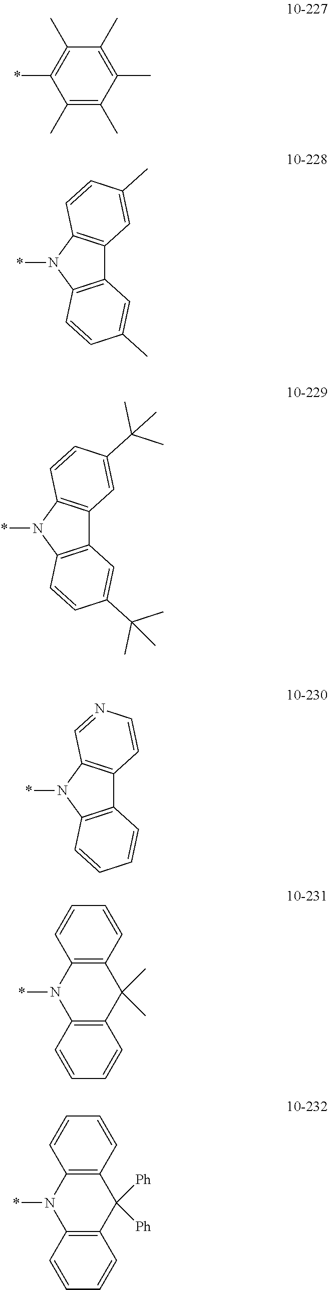

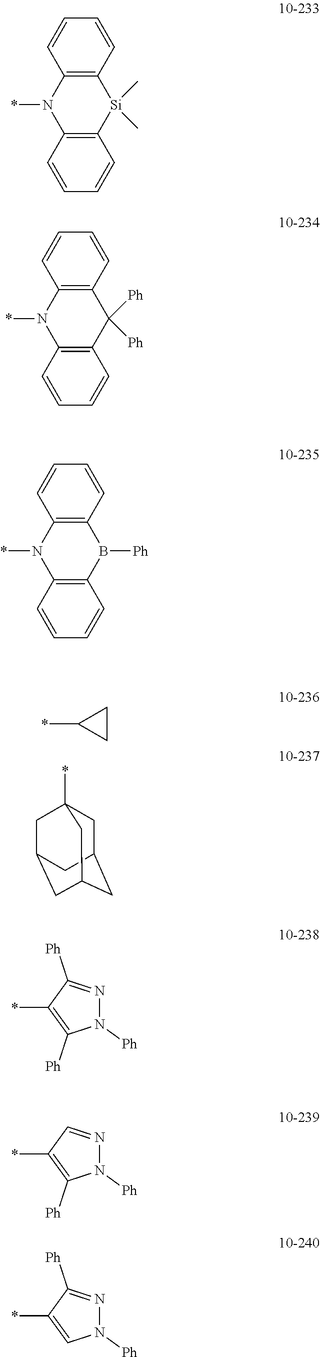

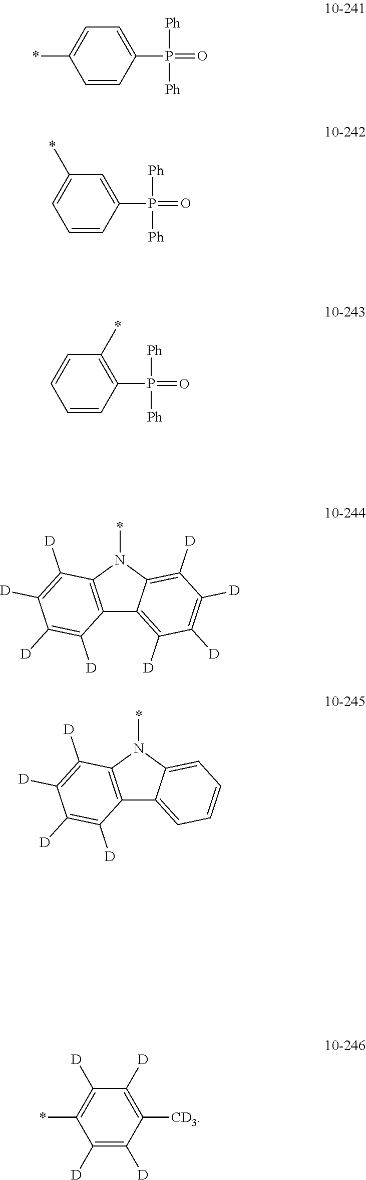 [SMILES: *c1ccc(P(=O)(c2ccccc2)c2ccccc2)cc1 ; *c1cccc(P(=O)(c2ccccc2)c2ccccc2)c1 ; *c1ccccc1P(=O)(c1ccccc1)c1ccccc1 ; *n1c2c([2H])c([2H])c([2H])c([2H])c2c2c([2H])c([2H])c([2H])c([2H])c21 ; *n1c2ccccc2c2c([2H])c([2H])c([2H])c([2H])c21 ; *c1c([2H])c([2H])c(C([2H])([2H])[2H])c([2H])c1[2H]]

In Formulae 9-1 to 9-19 and 10-1 to 10-246, * indicates a binding site to a neighboring atom, “Ph” represents a phenyl group, and “TMS” represents a trimethylsilyl group.

In Formulae 3-1 to 3-5, 502, and 503, a71 to a74 and a501 to a504 each indicate the number of R₇₁(s) to the number of R₇₄(s) and the number of R₅₀₁(s) to the number of R₅₀₄(s), respectively, and may each independently be an integer from 0 to 20. When a71 is 2 or greater, two or more of R₇₁ may be identical to or different from each other, when a72 is 2 or greater, two or more of R₇₂ may be identical to or different from each other, when a73 is 2 or greater, two or more of R₇₃ may be identical to or different from each other, when a74 is 2 or greater, two or more of R₇₄ may be identical to or different from each other, when a501 is 2 or greater, two or more of R₅₀₁ may be identical to or different from each other, when a502 is 2 or greater, two or more of R₅₀₂ may be identical to or different from each other, when a503 is 2 or greater, two or more of R₅₀₃ may be identical to or different from each other, and when a504 is 2 or greater, two or more of R₅₀₄ may be identical to or different from each other. a71 to a74 and a501 to a504 may each independently be an integer from 0 to 8.

In Formula 2, a group represented by *-(L₅₁)_{b51}-R₅₁ and a group represented by *-(L₅₂)_{b52}-R₅₂ may each not be a phenyl group.

In an embodiment, in Formula 2, a group represented by *-(L₅₁)_{b51}-R₅₁ and a group represented by *-(L₅₂)_{b52}-R₅₂ may be identical to each other.

In an embodiment, in Formula 2, a group represented by *-(L₅₁)_{b51}-R₅₁ and a group represented by *-(L₅₂)_{b52}-R₅₂ in Formula 2 may be different from each other.

In an embodiment, in Formula 2, b51 and b52 may each independently be 1, 2, or 3; and L₅₁ and L₅₂ may each independently be a benzene group, a pyridine group, a pyrimidine group, a pyridazine group, a pyrazine group, or a triazine group, each unsubstituted or substituted with at least one R₁₀ₐ.

In an embodiment, in Formula 2, R₅₁ and R₅₂ may each independently be a C₅-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —C(Q₁)(Q₂)(Q₃), or —Si(Q₁)(Q₂)(Q₃), and
Q₁ to Q₃ may each independently be a C₅-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

In an embodiment, a group represented by *-(L₅₁)_{b51}-R₅₁ in Formula 2 may be a group represented by one of Formulae CY51-1 to CY51-26, and/or
a group represented by *-(L₅₂)_{b52}-R₅₂ in Formula 2 may be a group represented by one of Formulae CY52-1 to CY52-26, and/or
a group represented by *-(L₅₃)_{b53}-R₅₃ in Formula 2 may be a group represented by one of Formulae CY53-1 to CY53-27, —C(Q₁)(Q₂)(Q₃), or —Si(Q₁)(Q₂)(Q₃):

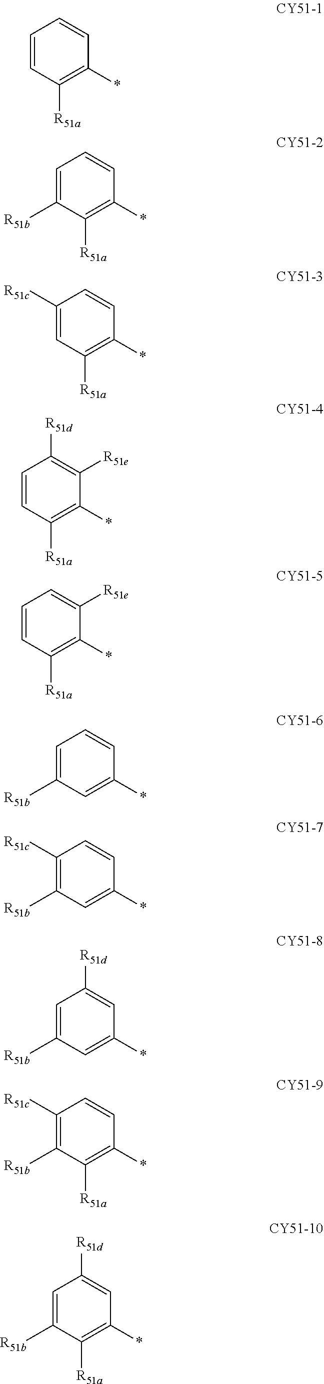

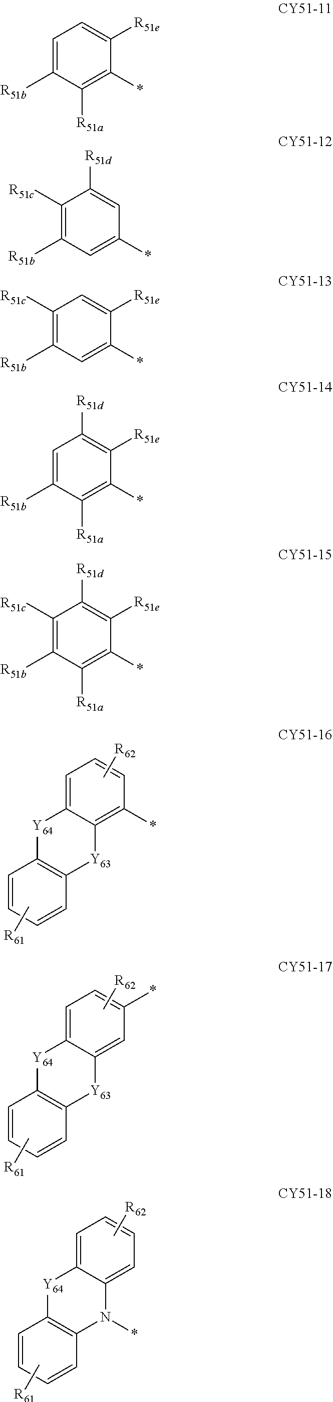

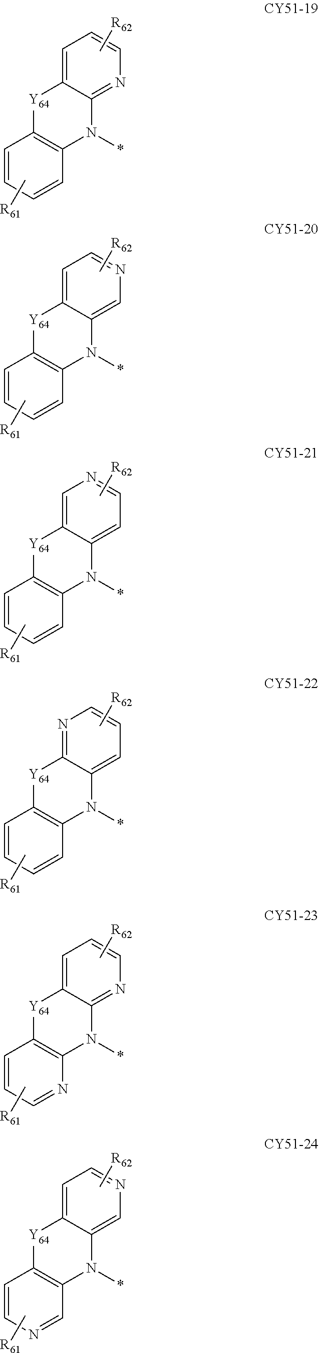

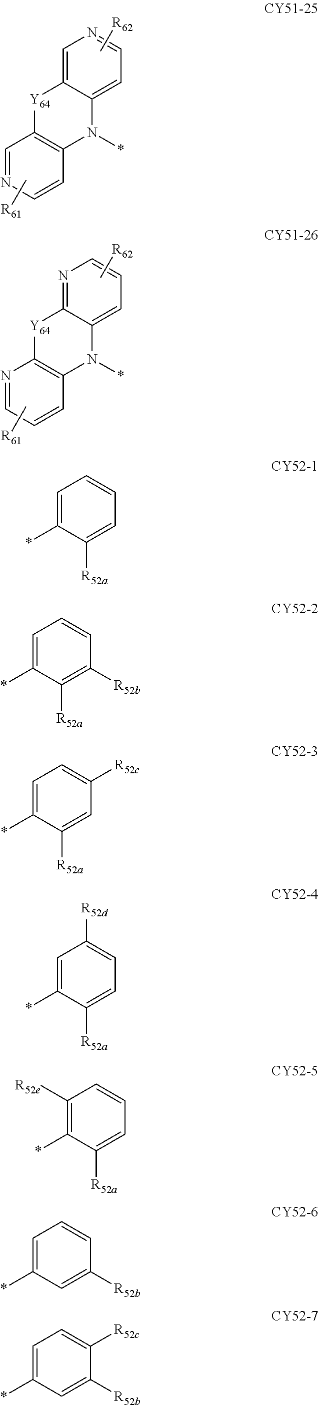

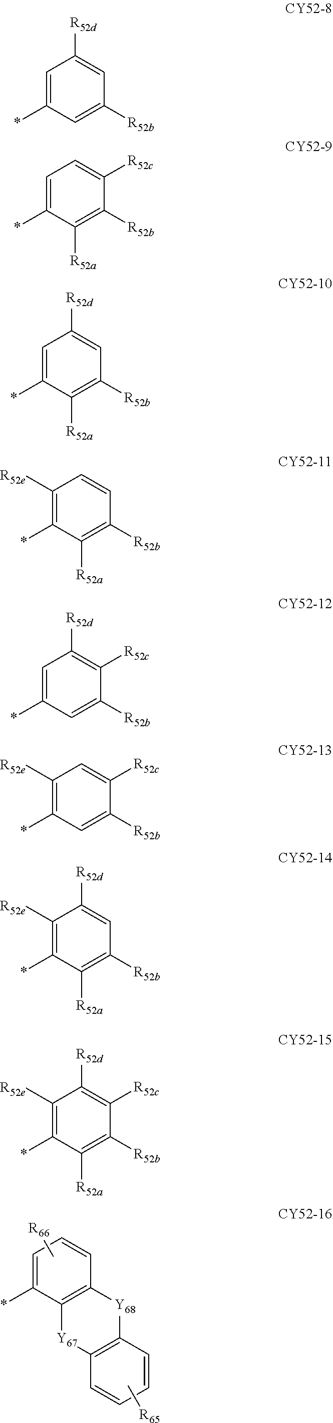

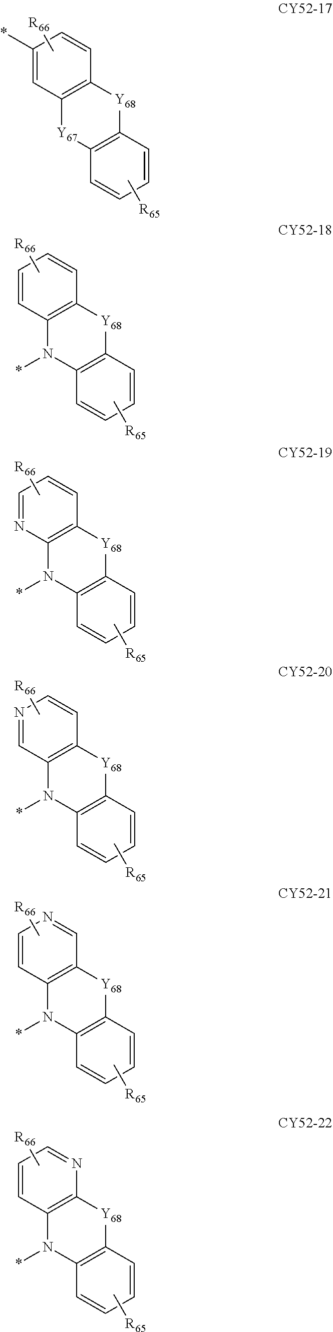

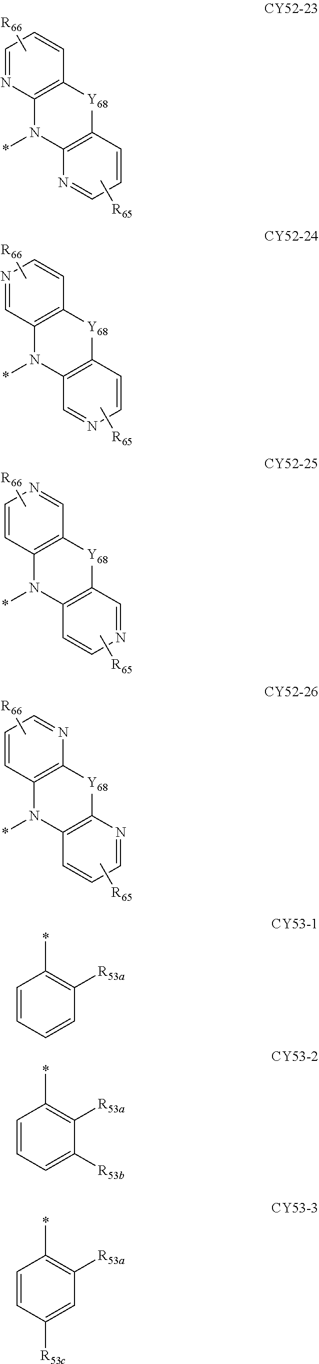

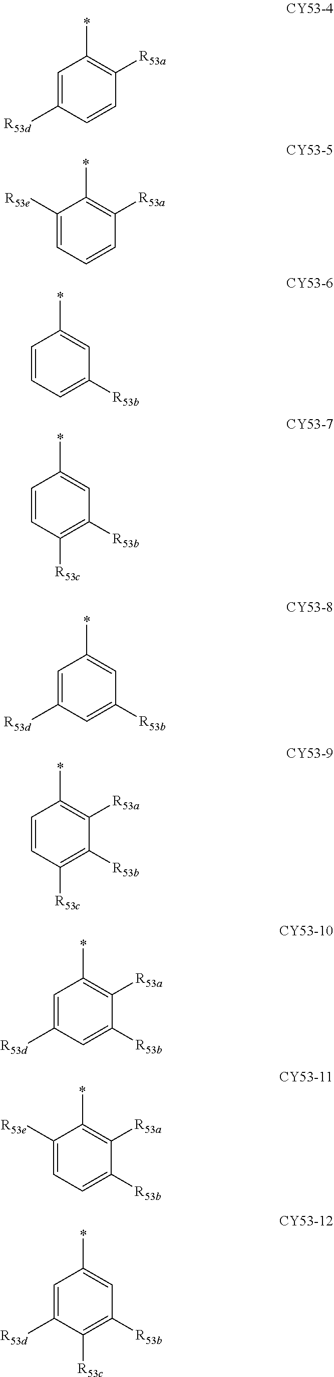

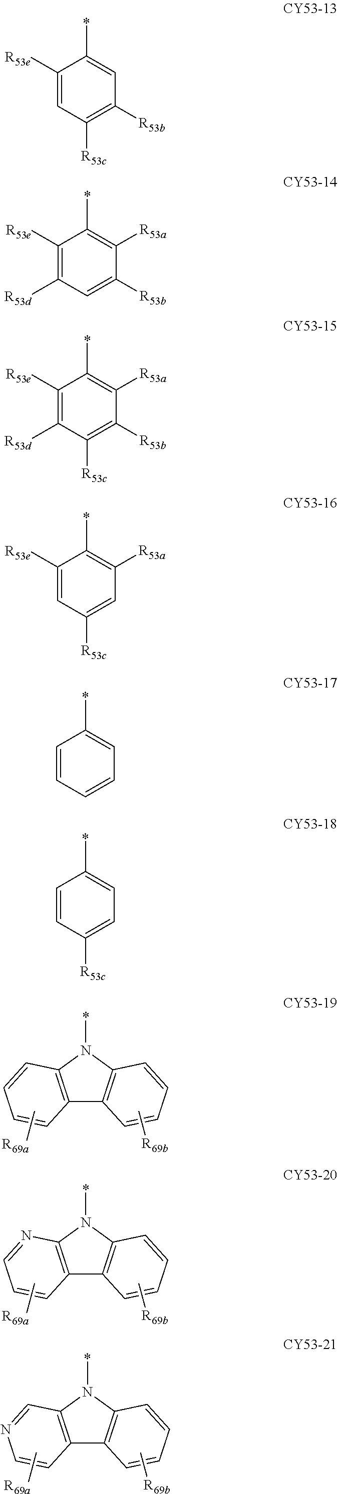

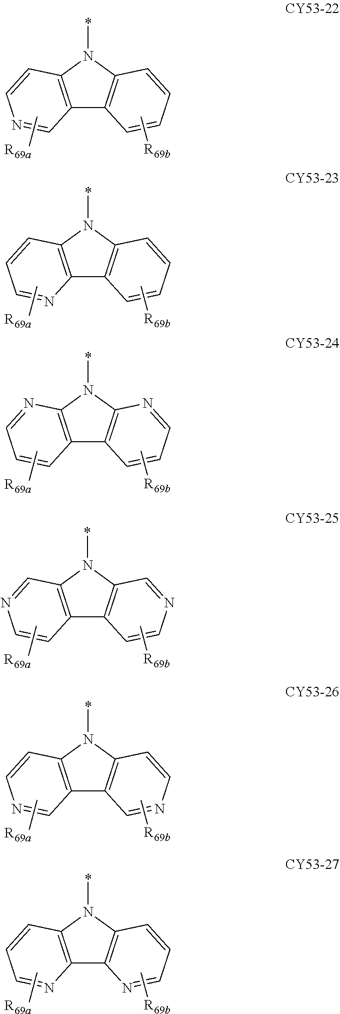

wherein in Formulae CY51-1 to CY51-26, CY52-1 to CY52-26, and CY53-1 to CY53-27,
Y₆₃ may be a single bond, O, S, N(R₆₃), B(R₆₃), C(R₆₃ₐ)(R_{63b}), or Si(R₆₃ₐ)(R_{63b}),
Y₆₄ may be a single bond, O, S, N(R₆₄), B(R₆₄), C(R₆₄ₐ)(R_{64b}), or Si(R₆₄ₐ)(R_{64b}),
Y₆₇ may be a single bond, O, S, N(R₆₇), B(R₆₇), C(R₆₇ₐ)(R_{67b}), or Si(R₆₇ₐ)(R_{67b}),
Y₆₈ may be a single bond, O, S, N(R₆₈), B(R₆₈), C(R₆₈ₐ)(R_{68b}), or Si(R₆₈ₐ)(R_{68b}),
Y₆₃ and Y₆₄ in Formulae CY51-16 and CY51-17 may not each be a single bond at the same time,
Y₆₇ and Y₆₈ in Formulae CY52-16 and CY52-17 may not each be a single bond at the same time,
R₅₁ₐ to R₅₁ₑ, R₆₁ to R₆₄, R₆₃ₐ, R_{63b}, R₆₄ₐ, and R_{64b} may each be the same as described in connection with R₅₁ as defined herein, except that R₅₁ₐ to R₅₁ₑ may not each be hydrogen,
R₅₂ₐ to R₅₂ₑ, R₆₅ to R₆₈, R₆₇ₐ, R_{67b}, R₆₈ₐ, and R_{68b} may each be the same as described in connection with R₅₂ as defined herein, except that R₅₂ₐ to R₅₂ₑ may not each be hydrogen,
R₅₃ₐ to R₅₃ₑ, R₆₉ₐ, and R_{69b} may each be the same as described in connection with R₅₂ as defined herein, except that R₅₃ₐ to R₅₃ₑ may not each be hydrogen, and
* indicates a binding site to a neighboring atom.

In embodiments, in Formulae CY51-1 to CY51-26 and Formulae CY52-1 to 52-26, R₅₁ₐ to R₅₁ₑ and R₅₂ₐ to R₅₂ₑ may each independently be:
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzoimidazolyl group, a benzofuranyl group, a benzothiophenyl group, a benzoisothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, an azafluorenyl group, an azadibenzosilolyl group, or a group represented by Formula 91, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzoimidazolyl group, a benzofuranyl group, a benzothiophenyl group, a benzoisothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, or any combination thereof; or
—C(Q₁)(Q₂)(Q₃) or —Si(Q₁)(Q₂)(Q₃),
Q₁ to Q₃ may each independently be a phenyl group, a naphthyl group, a pyridinyl group, a pyrimidinyl group, a pyridazinyl group, a pyrazinyl group, or a triazinyl group, each unsubstituted or substituted with deuterium, a C₁-C₁₀ alkyl group, a phenyl group, a biphenyl group, a pyridinyl group, a pyrimidinyl group, a pyridazinyl group, a pyrazinyl group, a triazinyl group, or any combination thereof,
in Formulae CY51-16 and CY51-17, Y63 may be O or S and Y64 may be Si(R₆₄ₐ)(R_{64b}), or Y₆₃ may be Si(R₆₃ₐ)(R_{63b}) and Y₆₄ may be O or S, and
in Formulae CY52-16 and CY52-17, Y₆₇ may be O or S, and Y₆₈ may be Si(R₆₈ₐ)(R_{68b}), or Y₆₇ may be Si(R₆₇ₐ)(R_{67b}), and Y₆₈ may be O or S.

In embodiments, in Formulae 3-1 to 3-5, L₈₁ to L₈₅ may each independently be:
a single bond;
*—C(Q₄)(Q₅)—*′ or *—Si(Q₄)(Q₅)—*′; or
a benzene group, a naphthalene group, an anthracene group, a phenanthrene group, a triphenylene group, a pyrene group, a chrysene group, a cyclopentadiene group, a furan group, a thiophene group, a silole group, an indene group, a fluorene group, an indole group, a carbazole group, a benzofuran group, a dibenzofuran group, a benzothiophene group, a dibenzothiophene group, a benzosilole group, a dibenzosilole group, an azafluorene group, an azacarbazole group, an azadibenzofuran group, an azadibenzothiophene group, an azadibenzosilole group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isooxazole group, a thiazole group, an isothiazole group, an oxadiazole group, a thiadiazole group, a benzopyrazole group, a benzimidazole group, a benzoxazole group, a benzothiazole group, a benzoxadiazole group, or a benzothiadiazole group, each unsubstituted or substituted with deuterium, —F, —Cl, —Br, —I, a hydroxyl group, a cyano group, a nitro group, a C₁-C₂₀ alkyl group, a C₁-C₂₀ alkoxy group, a phenyl group, a naphthyl group, a pyridinyl group, a pyrimidinyl group, a triazinyl group, a fluorenyl group, a dimethylfluorenyl group, a diphenylfluorenyl group, a carbazolyl group, a phenylcarbazolyl group, a dibenzofuranyl group, a dibenzothiophenyl group, a dibenzosilolyl group, a dimethyldibenzosilolyl group, a diphenyldibenzosilolyl group, —O(Q₃₁), —S(Q₃₁), —Si(Q₃₁)(Q₃₂)(Q₃₃), —N(Q₃₁)(Q₃₂), —B(Q₃₁)(Q₃₂), —P(Q₃₁)(Q₃₂), —C(═O)(Q₃₁), —S(═O)₂(Q₃₁), —P(═O)(Q₃₁)(Q₃₂), or any combination thereof, and
wherein Q₄, Q₅, and Q₃₁ to Q₃₃ may each independently be hydrogen, deuterium, a C₁-C₂₀ alkyl group, a C₁-C₂₀ alkoxy group, a phenyl group, a biphenyl group, a terphenyl group, a pyridinyl group, a pyrimidinyl group, a pyridazinyl group, a pyrazinyl group, or a triazinyl group.

In embodiments, in Formulae 3-1 and 3-2, a group represented by

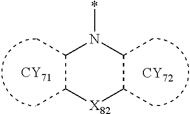

may be a group represented by one of Formulae CY71-1 (1) to CY71-1 (8), and/or
in Formulae 3-1 and 3-3, a group represented by

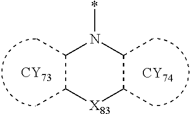

may be a group represented by one of Formulae CY71-2(1) to CY71-2(8), and/or
in Formulae 3-2 and 3-4, a group represented by

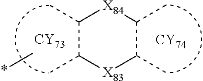

may be a group represented by one of Formulae CY71-3(1) to CY71-3(32), and/or
in Formulae 3-3 to 3-5, a group represented by

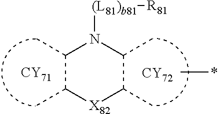

may be a group represented by one of Formulae CY71-4(1) to CY71-4(32), and/or
in Formula 3-5, a group represented by

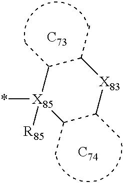

may be a group represented by one of Formulae CY71-5(1) to CY71-5(8):

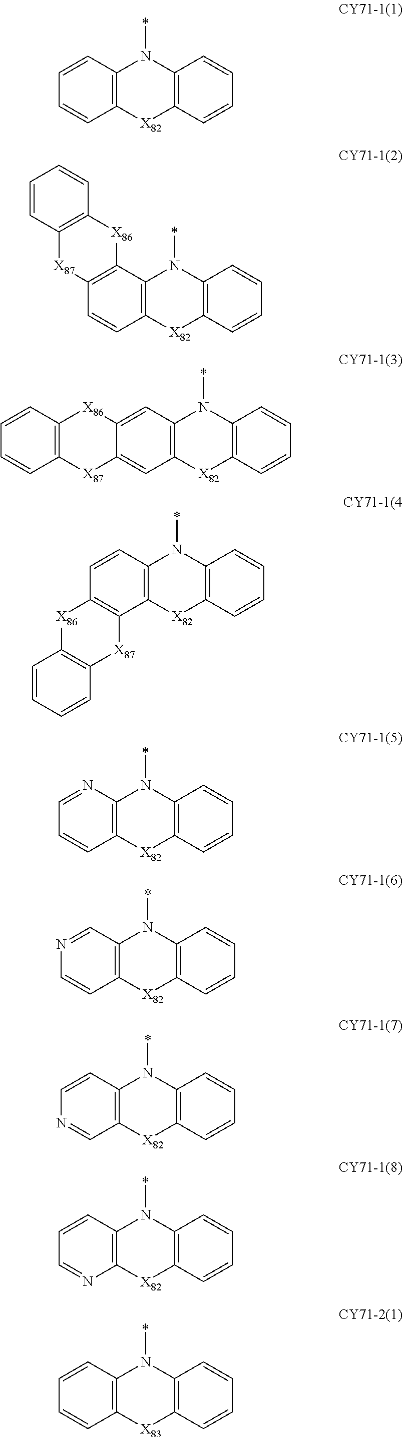

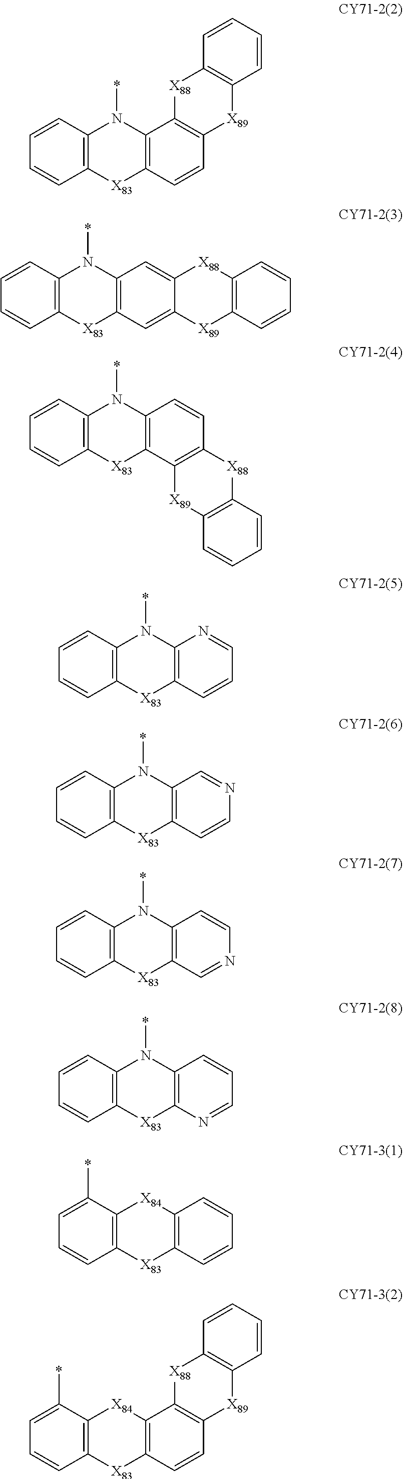

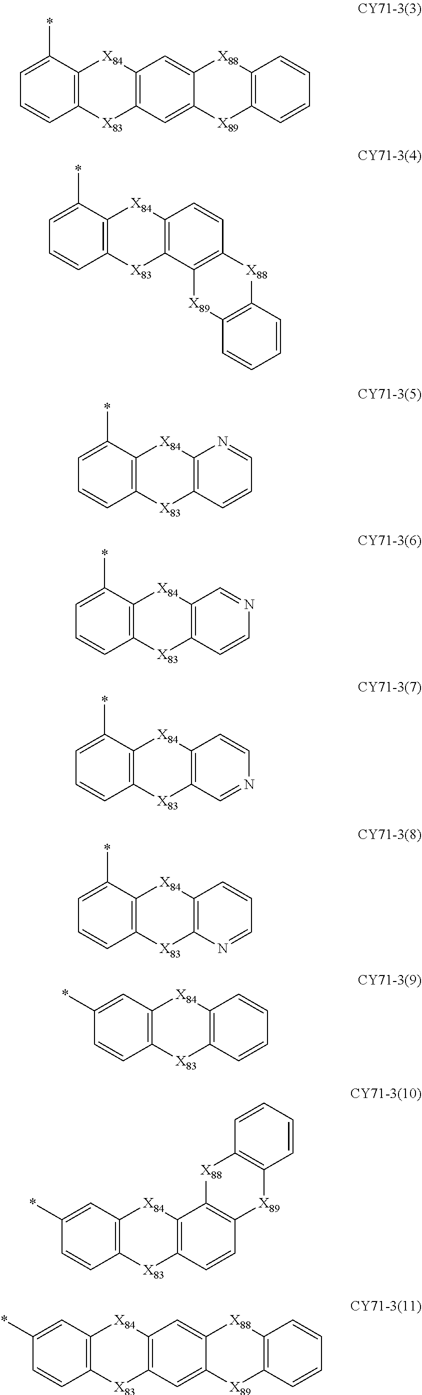

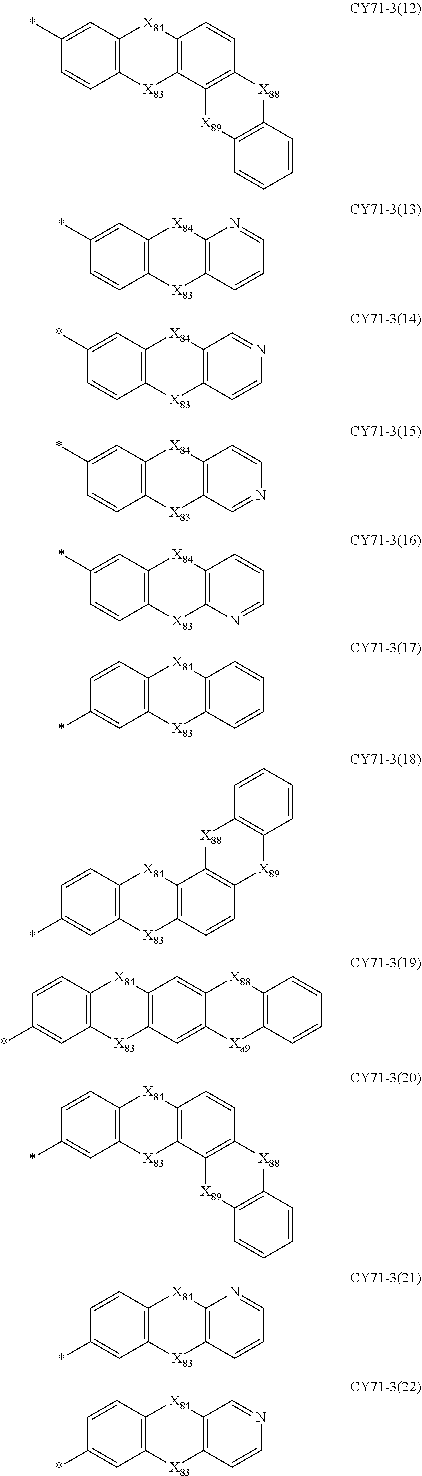

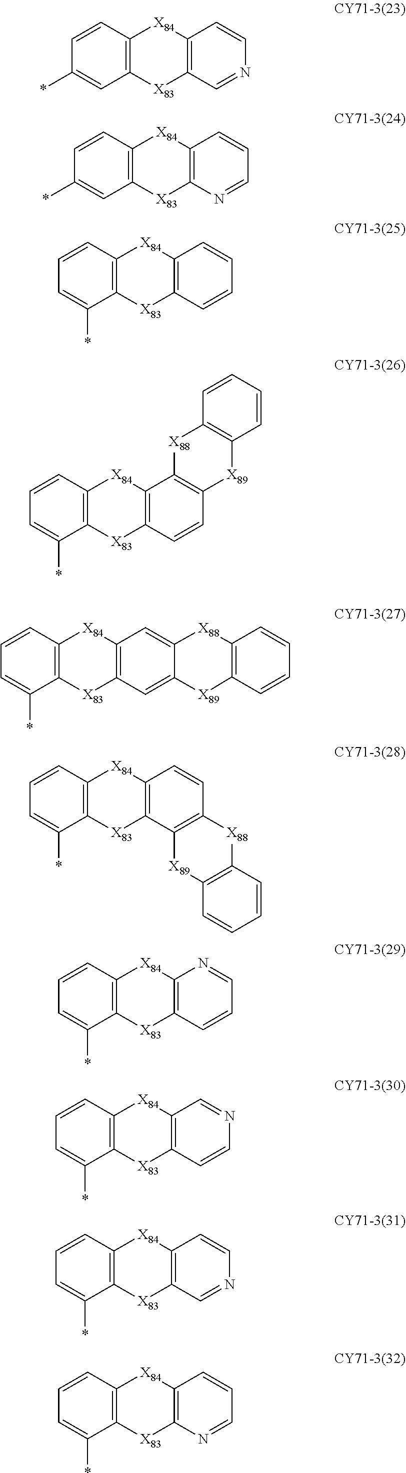

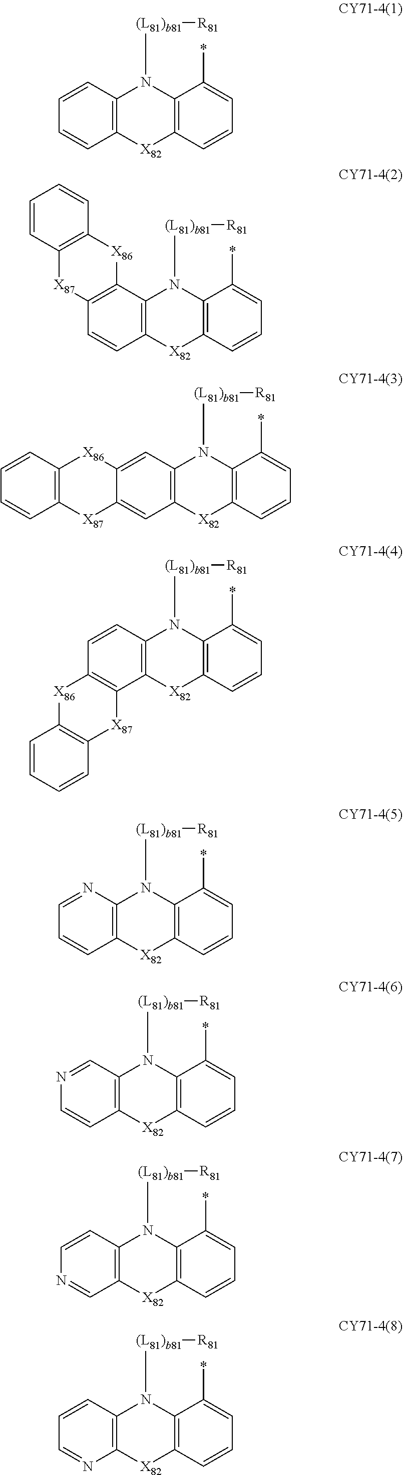

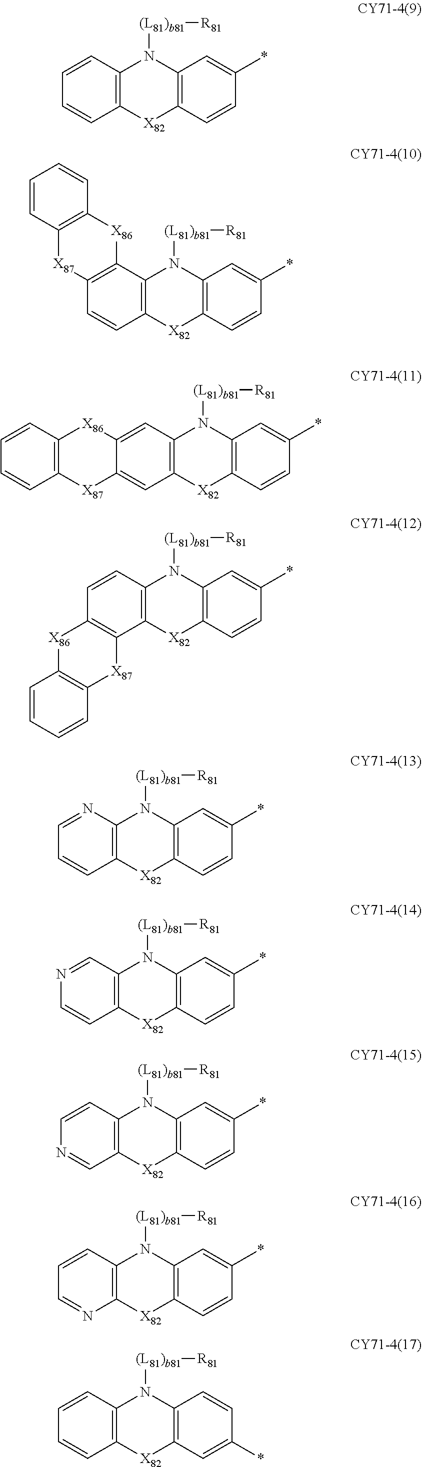

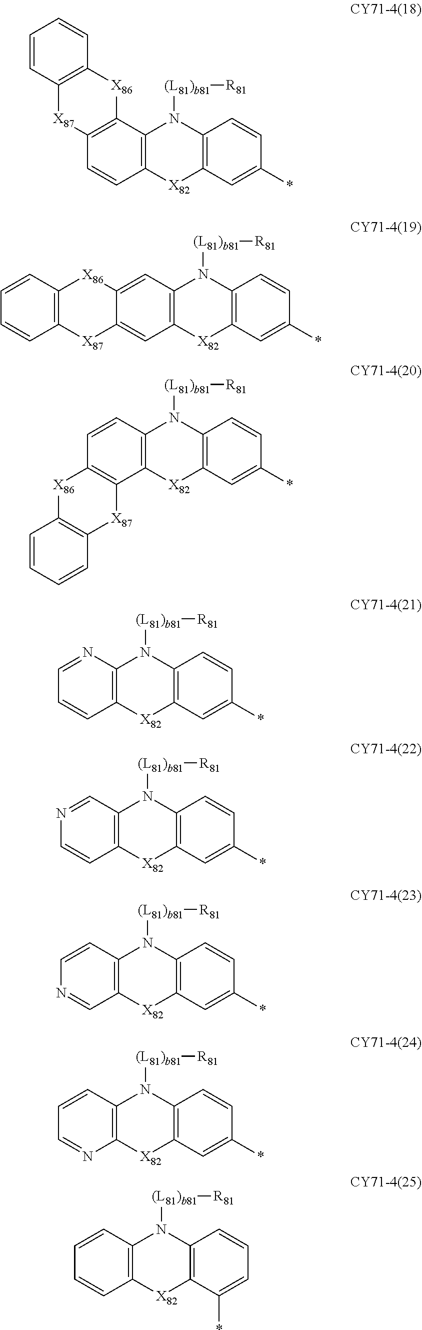

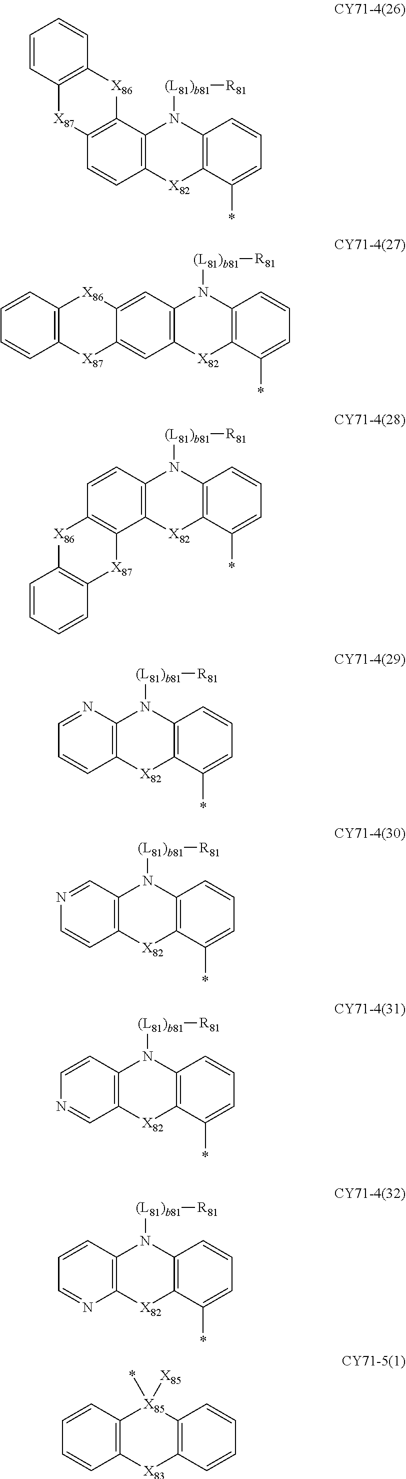

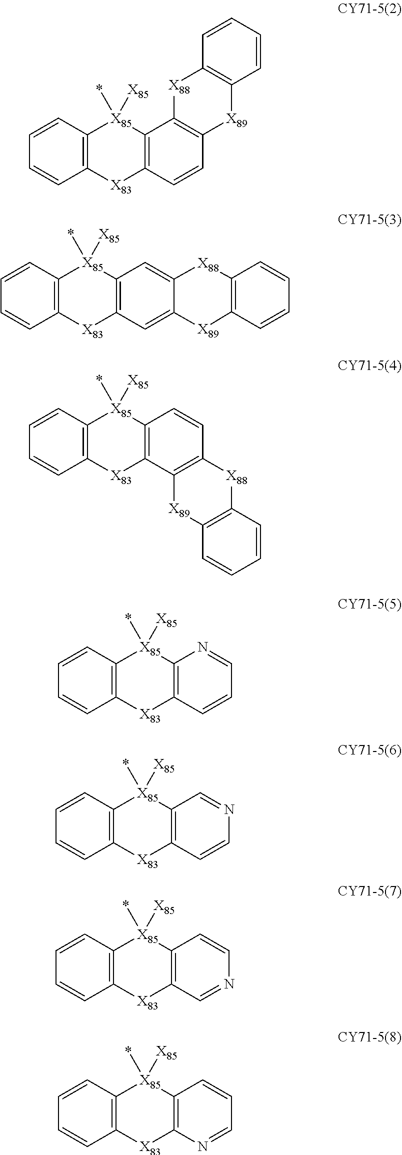

wherein in Formulae CY71-1(1) to CY71-1(8), CY71-2(1) to CY71-2(8), CY71-3(1) to CY71-3(32), CY71-4(1) to CY71-4(32), and CY71-5(1) to CY71-5(8),
X₈₁ to X₈₅, L₈₁, b81, R₈₁, and R₈₅ may respectively be the same as described herein,
X₈₆ may be a single bond, O, S, N(R₈₆), B(R₈₆), C(R₈₆ₐ) (R_{86b}), or Si(R₈₆ₐ)(R_{86b}),
X₈₇ may be a single bond, O, S, N(R₈₇), B(R₈₇), C(R₈₇ₐ)(R_{87b}), or Si(R₈₇ₐ)(R_{87b}),
in Formulae CY71-1(1) to CY71-1(8) and CY71-4(1) to CY71-4(32), X₈₆ and X₈₇ may not each be a single bond at the same time,
X₈₈ may be a single bond, O, S, N(R₈₈), B(R₈₈), C(R₈₈ₐ)(R_{88b}), or Si(R₈₈ₐ)(R_{88b}),
X₈₉ may be a single bond, O, S, N(R₈₉), B(R₈₉), C(R₈₉ₐ)(R_{89b}), or Si(R₈₉ₐ)(R_{89b}),
in Formulae CY71-2(1) to CY71-2(8), CY71-3(1) to CY71-3(32), and CY71-5(1) to CY71-5(8), X₈₈ and X₈₉ may not each be a single bond at the same time, and
R₈₆ to R₈₉, R₈₆ₐ, R_{86b}, R₈₇ₐ, R_{87b}, R₈₈ₐ, R_{88b}, R₈₉ₐ, and R_{89b} may each independently be the same as described in connection with R₈₁ as defined herein.

### Examples of Compounds

In an embodiment, the amine-based compound may be selected from Compounds 1 to 206:

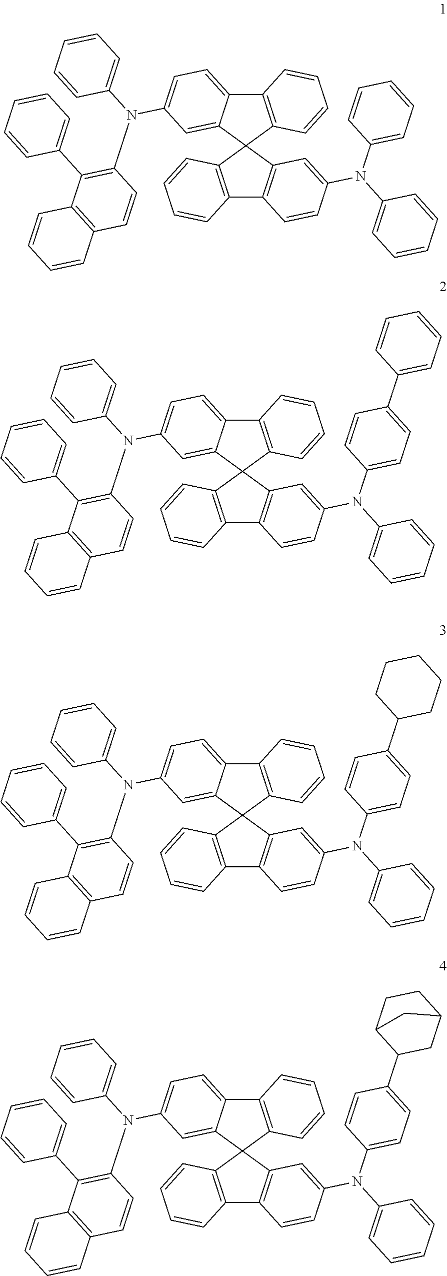 [SMILES: c1ccc(-c2c(N(c3ccccc3)c3ccc4c(c3)C3(c5ccccc5-c5ccc(N(c6ccccc6)c6ccccc6)cc53)c3ccccc3-4)ccc3ccccc23)cc1 ; c1ccc(-c2ccc(N(c3ccccc3)c3ccc4c(c3)C3(c5ccccc5-4)c4ccccc4-c4ccc(N(c5ccccc5)c5ccc6ccccc6c5-c5ccccc5)cc43)cc2)cc1 ; c1ccc(-c2c(N(c3ccccc3)c3ccc4c(c3)C3(c5ccccc5-c5ccc(N(c6ccccc6)c6ccc(C7CCCCC7)cc6)cc53)c3ccccc3-4)ccc3ccccc23)cc1 ; c1ccc(-c2c(N(c3ccccc3)c3ccc4c(c3)C3(c5ccccc5-c5ccc(N(c6ccccc6)c6ccc(C7CC8CCC7C8)cc6)cc53)c3ccccc3-4)ccc3ccccc23)cc1]

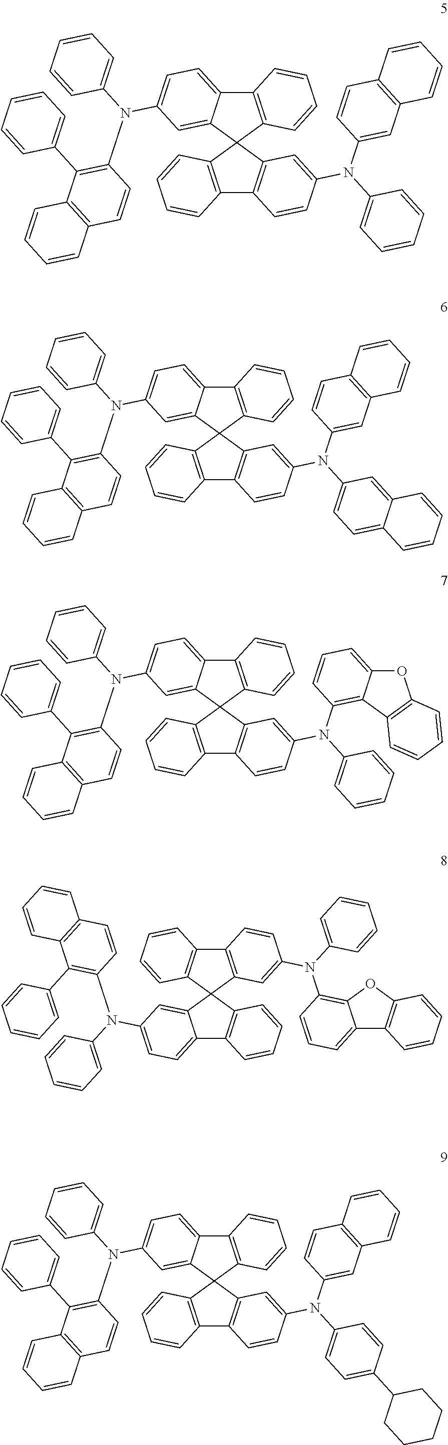

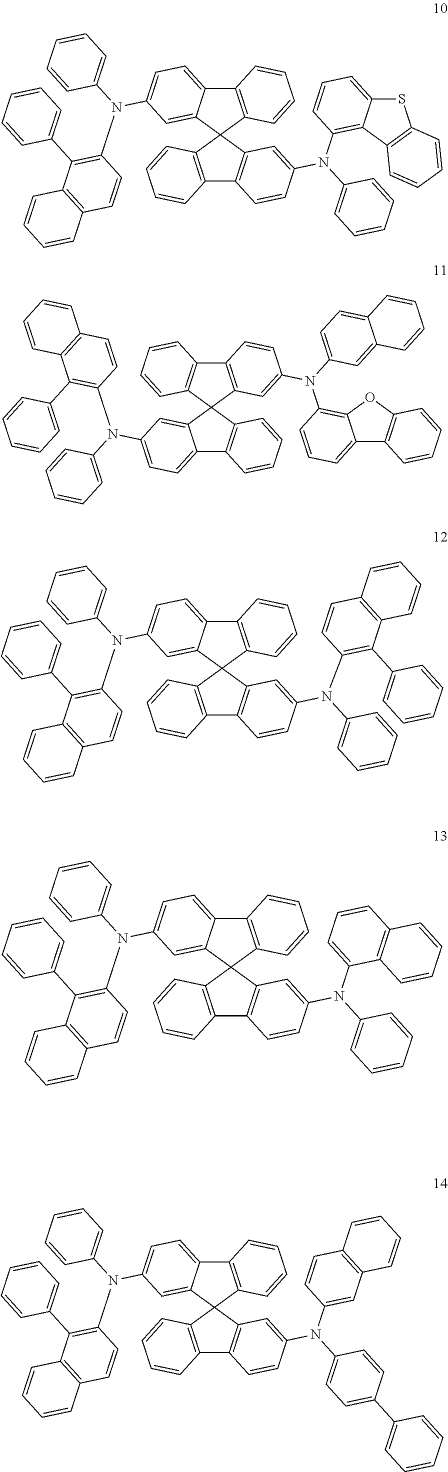

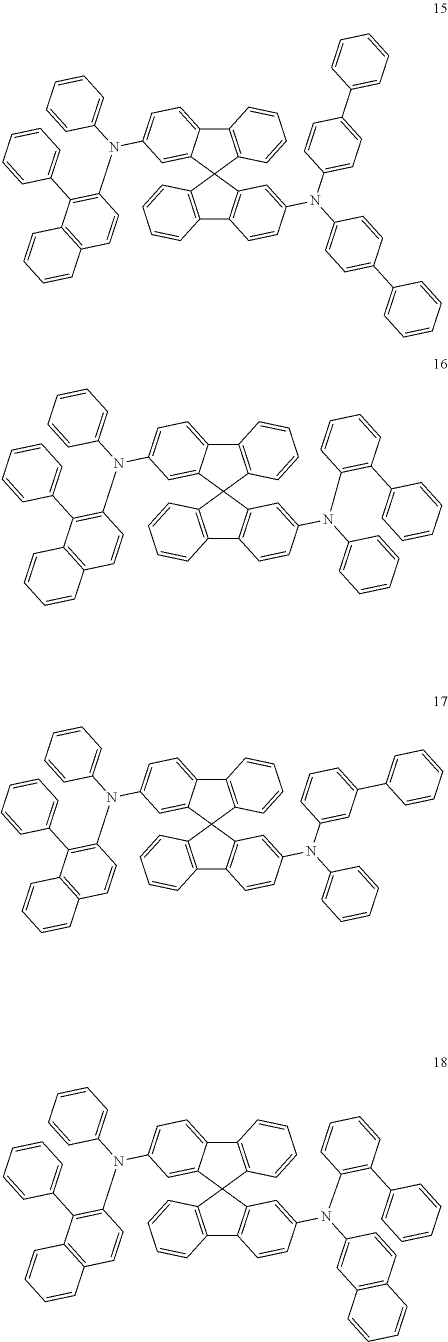 [SMILES: c1ccc(-c2ccc(N(c3ccc(-c4ccccc4)cc3)c3ccc4c(c3)C3(c5ccccc5-4)c4ccccc4-c4ccc(N(c5ccccc5)c5ccc6ccccc6c5-c5ccccc5)cc43)cc2)cc1 ; c1ccc(-c2ccccc2N(c2ccccc2)c2ccc3c(c2)C2(c4ccccc4-3)c3ccccc3-c3ccc(N(c4ccccc4)c4ccc5ccccc5c4-c4ccccc4)cc32)cc1 ; c1ccc(-c2cccc(N(c3ccccc3)c3ccc4c(c3)C3(c5ccccc5-4)c4ccccc4-c4ccc(N(c5ccccc5)c5ccc6ccccc6c5-c5ccccc5)cc43)c2)cc1 ; c1ccc(-c2ccccc2N(c2ccc3c(c2)C2(c4ccccc4-3)c3ccccc3-c3ccc(N(c4ccccc4)c4ccc5ccccc5c4-c4ccccc4)cc32)c2ccc3ccccc3c2)cc1]

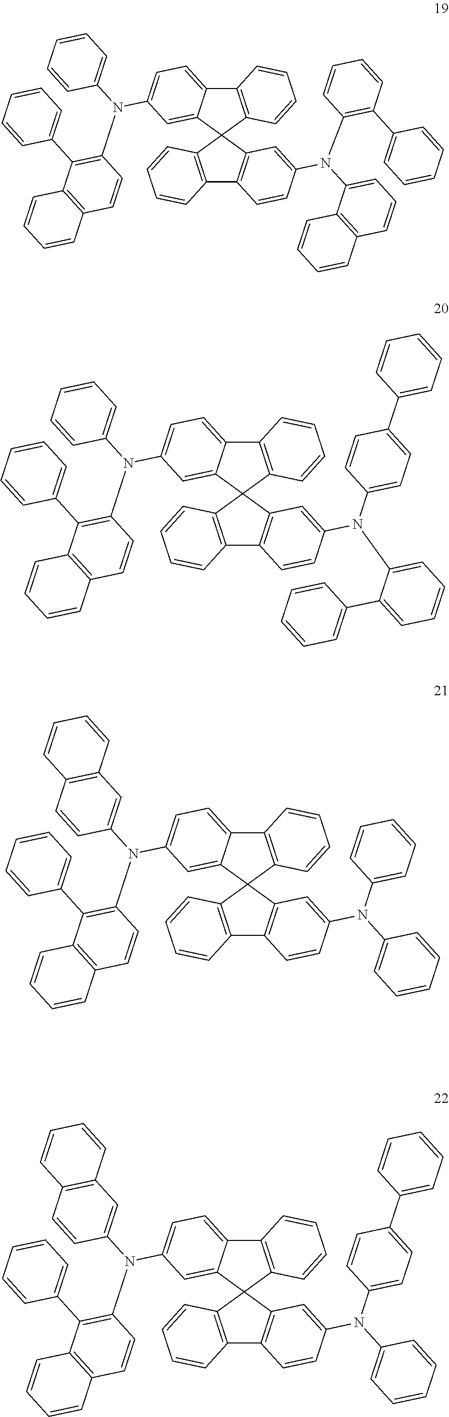 [SMILES: c1ccc(-c2ccccc2N(c2ccc3c(c2)C2(c4ccccc4-c4ccc(N(c5ccccc5)c5ccc6ccccc6c5-c5ccccc5)cc42)c2ccccc2-3)c2cccc3ccccc23)cc1 ; c1ccc(-c2ccc(N(c3ccc4c(c3)C3(c5ccccc5-4)c4ccccc4-c4ccc(N(c5ccccc5)c5ccc6ccccc6c5-c5ccccc5)cc43)c3ccccc3-c3ccccc3)cc2)cc1 ; c1ccc(-c2c(N(c3ccc4c(c3)C3(c5ccccc5-c5ccc(N(c6ccccc6)c6ccccc6)cc53)c3ccccc3-4)c3ccc4ccccc4c3)ccc3ccccc23)cc1 ; c1ccc(-c2ccc(N(c3ccccc3)c3ccc4c(c3)C3(c5ccccc5-4)c4ccccc4-c4ccc(N(c5ccc6ccccc6c5)c5ccc6ccccc6c5-c5ccccc5)cc43)cc2)cc1]

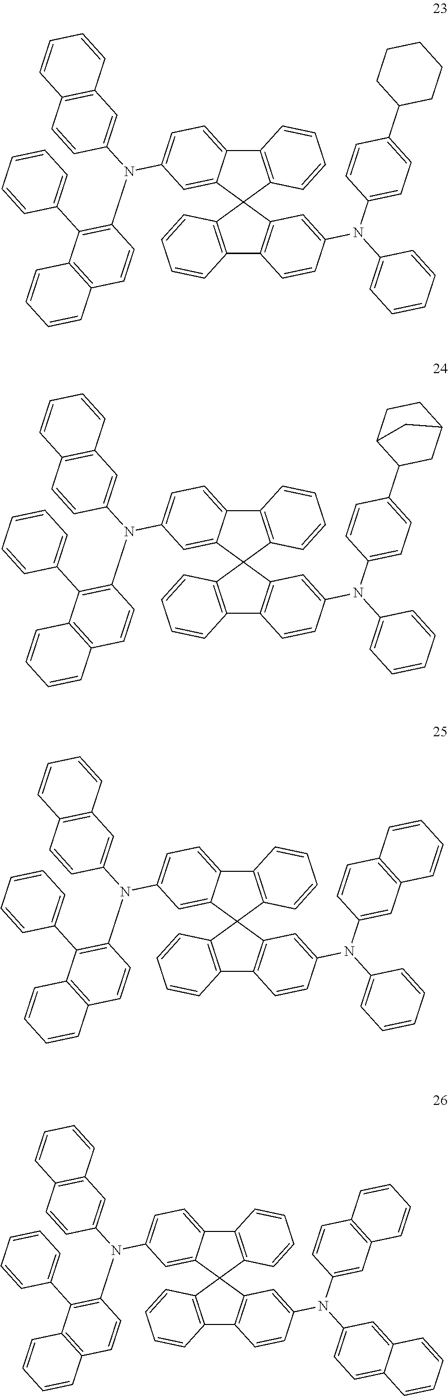 [SMILES: c1ccc(-c2c(N(c3ccc4c(c3)C3(c5ccccc5-c5ccc(N(c6ccccc6)c6ccc(C7CCCCC7)cc6)cc53)c3ccccc3-4)c3ccc4ccccc4c3)ccc3ccccc23)cc1 ; c1ccc(-c2c(N(c3ccc4c(c3)C3(c5ccccc5-c5ccc(N(c6ccccc6)c6ccc(C7CC8CCC7C8)cc6)cc53)c3ccccc3-4)c3ccc4ccccc4c3)ccc3ccccc23)cc1 ; c1ccc(-c2c(N(c3ccc4c(c3)C3(c5ccccc5-c5ccc(N(c6ccccc6)c6ccc7ccccc7c6)cc53)c3ccccc3-4)c3ccc4ccccc4c3)ccc3ccccc23)cc1 ; c1ccc(-c2c(N(c3ccc4c(c3)C3(c5ccccc5-c5ccc(N(c6ccc7ccccc7c6)c6ccc7ccccc7c6)cc53)c3ccccc3-4)c3ccc4ccccc4c3)ccc3ccccc23)cc1]

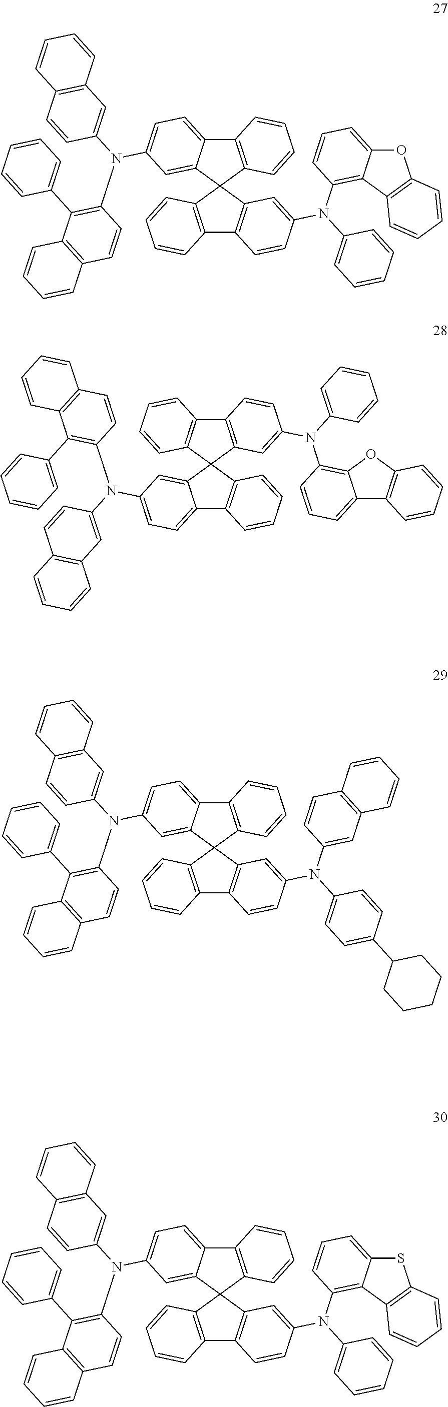 [SMILES: c1ccc(-c2c(N(c3ccc4c(c3)C3(c5ccccc5-4)c4ccccc4-c4ccc(N(c5ccccc5)c5cccc6oc7ccccc7c56)cc43)c3ccc4ccccc4c3)ccc3ccccc23)cc1 ; c1ccc(-c2c(N(c3ccc4c(c3)C3(c5ccccc5-4)c4ccccc4-c4ccc(N(c5ccccc5)c5cccc6c5oc5ccccc56)cc43)c3ccc4ccccc4c3)ccc3ccccc23)cc1 ; c1ccc(-c2c(N(c3ccc4c(c3)C3(c5ccccc5-c5ccc(N(c6ccc(C7CCCCC7)cc6)c6ccc7ccccc7c6)cc53)c3ccccc3-4)c3ccc4ccccc4c3)ccc3ccccc23)cc1 ; c1ccc(-c2c(N(c3ccc4c(c3)C3(c5ccccc5-4)c4ccccc4-c4ccc(N(c5ccccc5)c5cccc6sc7ccccc7c56)cc43)c3ccc4ccccc4c3)ccc3ccccc23)cc1]

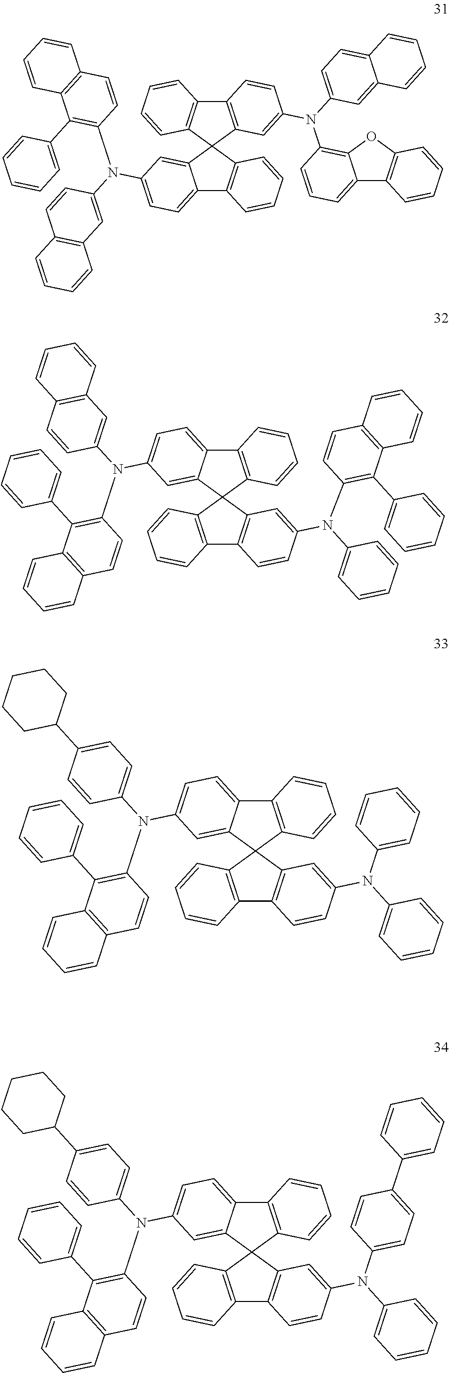 [SMILES: c1ccc(-c2c(N(c3ccc4c(c3)C3(c5ccccc5-4)c4ccccc4-c4ccc(N(c5ccc6ccccc6c5)c5cccc6c5oc5ccccc56)cc43)c3ccc4ccccc4c3)ccc3ccccc23)cc1 ; c1ccc(-c2c(N(c3ccccc3)c3ccc4c(c3)C3(c5ccccc5-4)c4ccccc4-c4ccc(N(c5ccc6ccccc6c5)c5ccc6ccccc6c5-c5ccccc5)cc43)ccc3ccccc23)cc1 ; c1ccc(-c2c(N(c3ccc(C4CCCCC4)cc3)c3ccc4c(c3)C3(c5ccccc5-c5ccc(N(c6ccccc6)c6ccccc6)cc53)c3ccccc3-4)ccc3ccccc23)cc1 ; c1ccc(-c2ccc(N(c3ccccc3)c3ccc4c(c3)C3(c5ccccc5-4)c4ccccc4-c4ccc(N(c5ccc(C6CCCCC6)cc5)c5ccc6ccccc6c5-c5ccccc5)cc43)cc2)cc1]

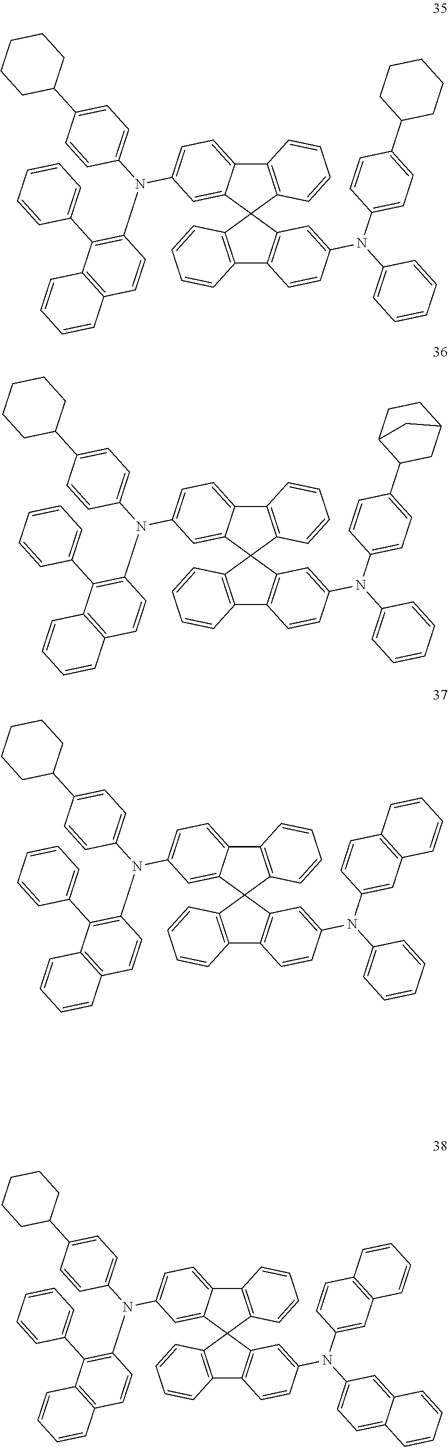 [SMILES: c1ccc(-c2c(N(c3ccc(C4CCCCC4)cc3)c3ccc4c(c3)C3(c5ccccc5-c5ccc(N(c6ccccc6)c6ccc(C7CCCCC7)cc6)cc53)c3ccccc3-4)ccc3ccccc23)cc1 ; c1ccc(-c2c(N(c3ccc(C4CCCCC4)cc3)c3ccc4c(c3)C3(c5ccccc5-c5ccc(N(c6ccccc6)c6ccc(C7CC8CCC7C8)cc6)cc53)c3ccccc3-4)ccc3ccccc23)cc1 ; c1ccc(-c2c(N(c3ccc(C4CCCCC4)cc3)c3ccc4c(c3)C3(c5ccccc5-c5ccc(N(c6ccccc6)c6ccc7ccccc7c6)cc53)c3ccccc3-4)ccc3ccccc23)cc1 ; c1ccc(-c2c(N(c3ccc(C4CCCCC4)cc3)c3ccc4c(c3)C3(c5ccccc5-c5ccc(N(c6ccc7ccccc7c6)c6ccc7ccccc7c6)cc53)c3ccccc3-4)ccc3ccccc23)cc1]

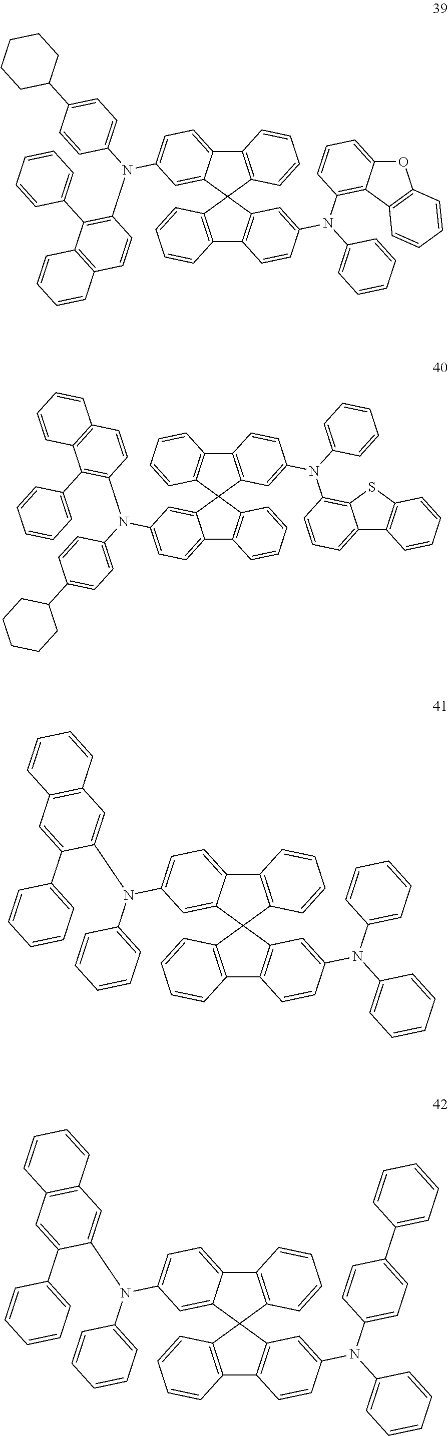 [SMILES: c1ccc(-c2c(N(c3ccc(C4CCCCC4)cc3)c3ccc4c(c3)C3(c5ccccc5-4)c4ccccc4-c4ccc(N(c5ccccc5)c5cccc6oc7ccccc7c56)cc43)ccc3ccccc23)cc1 ; c1ccc(-c2c(N(c3ccc(C4CCCCC4)cc3)c3ccc4c(c3)C3(c5ccccc5-4)c4ccccc4-c4ccc(N(c5ccccc5)c5cccc6c5sc5ccccc56)cc43)ccc3ccccc23)cc1 ; c1ccc(-c2cc3ccccc3cc2N(c2ccccc2)c2ccc3c(c2)C2(c4ccccc4-c4ccc(N(c5ccccc5)c5ccccc5)cc42)c2ccccc2-3)cc1 ; c1ccc(-c2ccc(N(c3ccccc3)c3ccc4c(c3)C3(c5ccccc5-4)c4ccccc4-c4ccc(N(c5ccccc5)c5cc6ccccc6cc5-c5ccccc5)cc43)cc2)cc1]

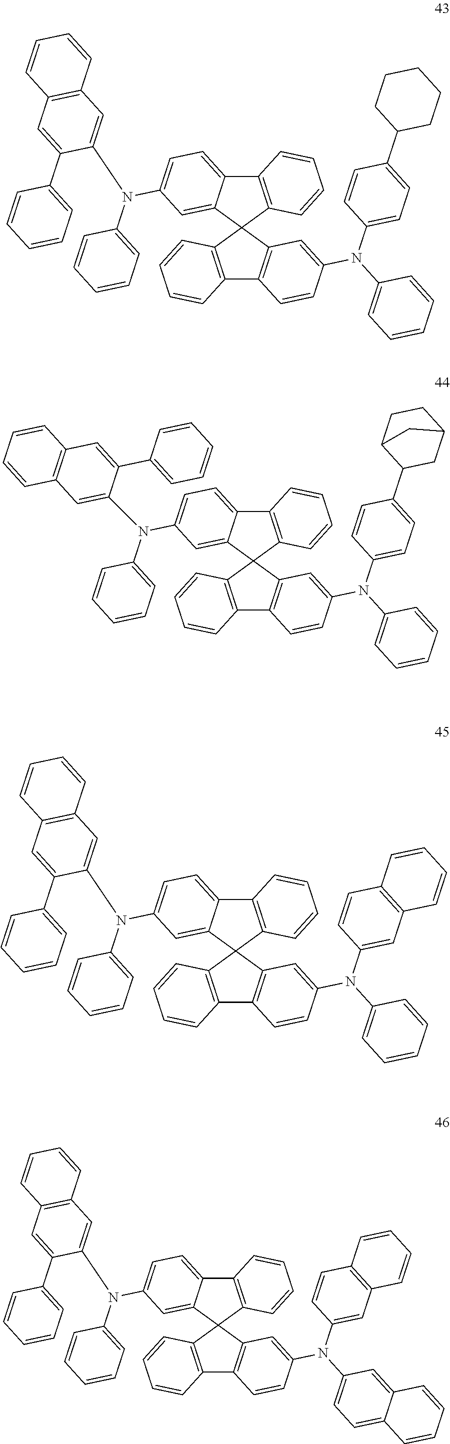 [SMILES: c1ccc(-c2cc3ccccc3cc2N(c2ccccc2)c2ccc3c(c2)C2(c4ccccc4-c4ccc(N(c5ccccc5)c5ccc(C6CCCCC6)cc5)cc42)c2ccccc2-3)cc1 ; c1ccc(-c2cc3ccccc3cc2N(c2ccccc2)c2ccc3c(c2)C2(c4ccccc4-c4ccc(N(c5ccccc5)c5ccc(C6CC7CCC6C7)cc5)cc42)c2ccccc2-3)cc1 ; c1ccc(-c2cc3ccccc3cc2N(c2ccccc2)c2ccc3c(c2)C2(c4ccccc4-c4ccc(N(c5ccccc5)c5ccc6ccccc6c5)cc42)c2ccccc2-3)cc1 ; c1ccc(-c2cc3ccccc3cc2N(c2ccccc2)c2ccc3c(c2)C2(c4ccccc4-c4ccc(N(c5ccc6ccccc6c5)c5ccc6ccccc6c5)cc42)c2ccccc2-3)cc1]

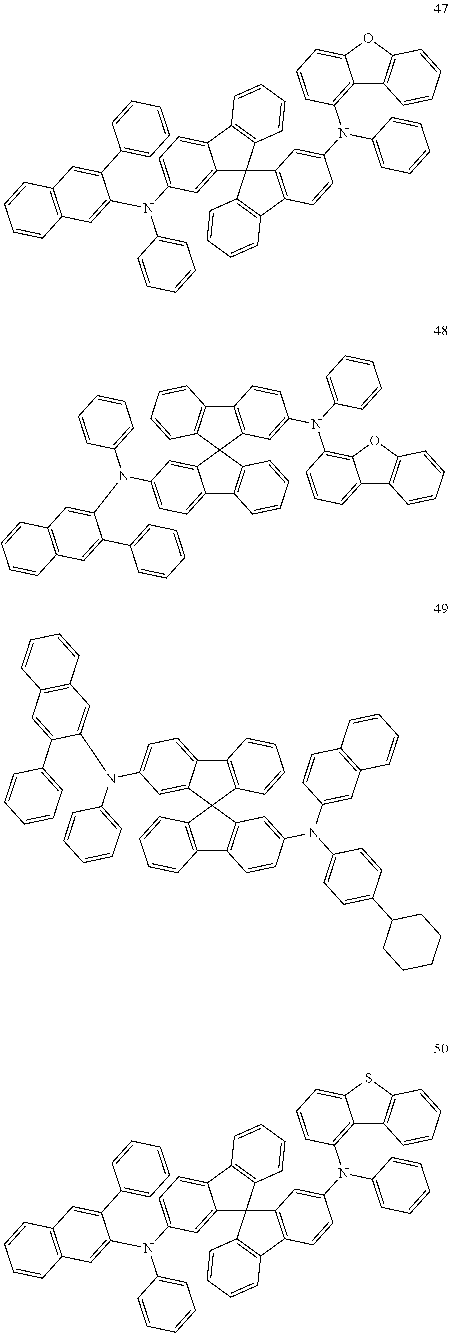 [SMILES: c1ccc(-c2cc3ccccc3cc2N(c2ccccc2)c2ccc3c(c2)C2(c4ccccc4-3)c3ccccc3-c3ccc(N(c4ccccc4)c4cccc5oc6ccccc6c45)cc32)cc1 ; c1ccc(-c2cc3ccccc3cc2N(c2ccccc2)c2ccc3c(c2)C2(c4ccccc4-3)c3ccccc3-c3ccc(N(c4ccccc4)c4cccc5c4oc4ccccc45)cc32)cc1 ; c1ccc(-c2cc3ccccc3cc2N(c2ccccc2)c2ccc3c(c2)C2(c4ccccc4-c4ccc(N(c5ccc(C6CCCCC6)cc5)c5ccc6ccccc6c5)cc42)c2ccccc2-3)cc1 ; c1ccc(-c2cc3ccccc3cc2N(c2ccccc2)c2ccc3c(c2)C2(c4ccccc4-3)c3ccccc3-c3ccc(N(c4ccccc4)c4cccc5sc6ccccc6c45)cc32)cc1]

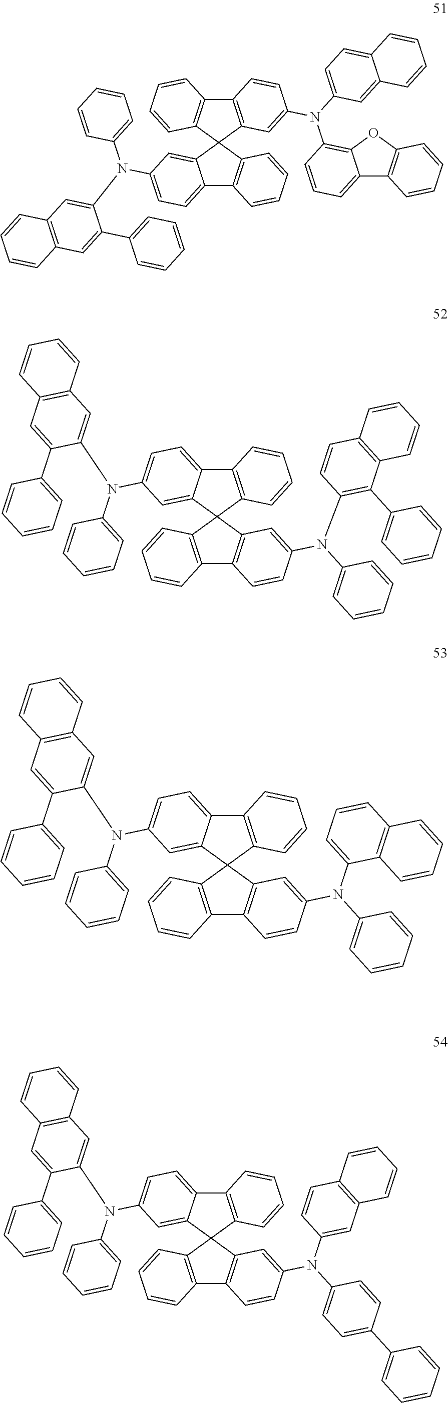 [SMILES: c1ccc(-c2cc3ccccc3cc2N(c2ccccc2)c2ccc3c(c2)C2(c4ccccc4-3)c3ccccc3-c3ccc(N(c4ccc5ccccc5c4)c4cccc5c4oc4ccccc45)cc32)cc1 ; c1ccc(-c2cc3ccccc3cc2N(c2ccccc2)c2ccc3c(c2)C2(c4ccccc4-3)c3ccccc3-c3ccc(N(c4ccccc4)c4ccc5ccccc5c4-c4ccccc4)cc32)cc1 ; c1ccc(-c2cc3ccccc3cc2N(c2ccccc2)c2ccc3c(c2)C2(c4ccccc4-3)c3ccccc3-c3ccc(N(c4ccccc4)c4cccc5ccccc45)cc32)cc1 ; c1ccc(-c2ccc(N(c3ccc4c(c3)C3(c5ccccc5-4)c4ccccc4-c4ccc(N(c5ccccc5)c5cc6ccccc6cc5-c5ccccc5)cc43)c3ccc4ccccc4c3)cc2)cc1]

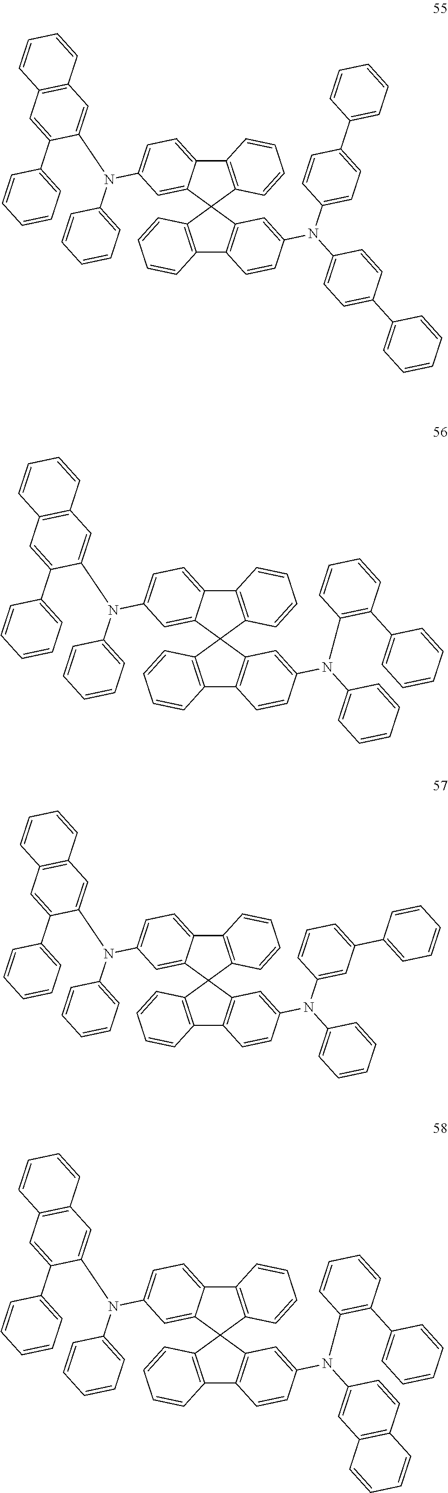 [SMILES: c1ccc(-c2ccc(N(c3ccc(-c4ccccc4)cc3)c3ccc4c(c3)C3(c5ccccc5-4)c4ccccc4-c4ccc(N(c5ccccc5)c5cc6ccccc6cc5-c5ccccc5)cc43)cc2)cc1 ; c1ccc(-c2ccccc2N(c2ccccc2)c2ccc3c(c2)C2(c4ccccc4-3)c3ccccc3-c3ccc(N(c4ccccc4)c4cc5ccccc5cc4-c4ccccc4)cc32)cc1 ; c1ccc(-c2cccc(N(c3ccccc3)c3ccc4c(c3)C3(c5ccccc5-4)c4ccccc4-c4ccc(N(c5ccccc5)c5cc6ccccc6cc5-c5ccccc5)cc43)c2)cc1 ; c1ccc(-c2ccccc2N(c2ccc3c(c2)C2(c4ccccc4-3)c3ccccc3-c3ccc(N(c4ccccc4)c4cc5ccccc5cc4-c4ccccc4)cc32)c2ccc3ccccc3c2)cc1]

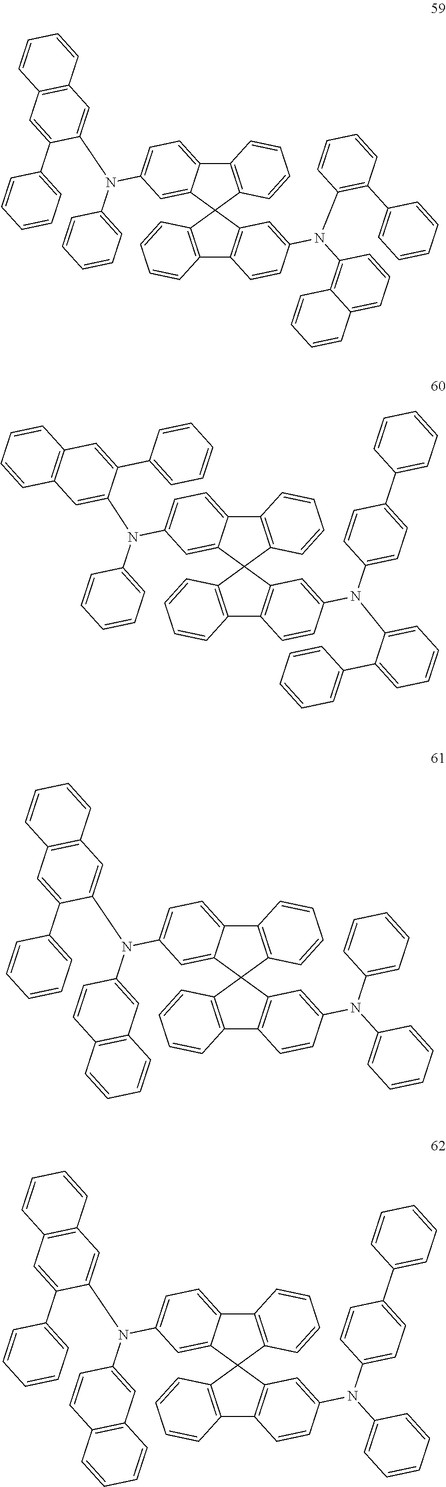 [SMILES: c1ccc(-c2cc3ccccc3cc2N(c2ccccc2)c2ccc3c(c2)C2(c4ccccc4-3)c3ccccc3-c3ccc(N(c4ccccc4-c4ccccc4)c4cccc5ccccc45)cc32)cc1 ; c1ccc(-c2ccc(N(c3ccc4c(c3)C3(c5ccccc5-4)c4ccccc4-c4ccc(N(c5ccccc5)c5cc6ccccc6cc5-c5ccccc5)cc43)c3ccccc3-c3ccccc3)cc2)cc1 ; c1ccc(-c2cc3ccccc3cc2N(c2ccc3c(c2)C2(c4ccccc4-c4ccc(N(c5ccccc5)c5ccccc5)cc42)c2ccccc2-3)c2ccc3ccccc3c2)cc1 ; c1ccc(-c2ccc(N(c3ccccc3)c3ccc4c(c3)C3(c5ccccc5-4)c4ccccc4-c4ccc(N(c5ccc6ccccc6c5)c5cc6ccccc6cc5-c5ccccc5)cc43)cc2)cc1]

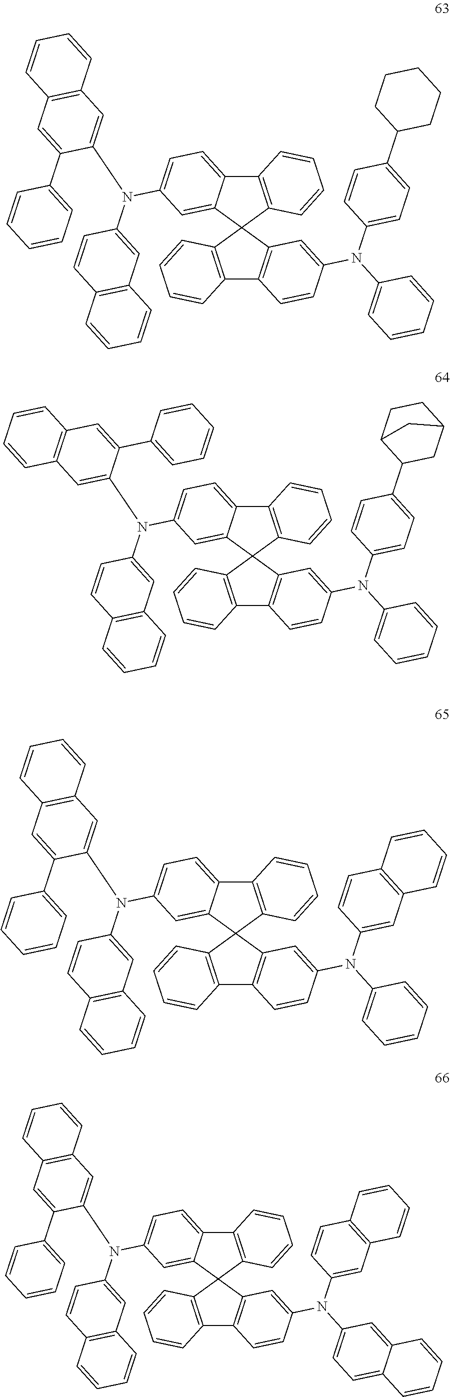 [SMILES: c1ccc(-c2cc3ccccc3cc2N(c2ccc3c(c2)C2(c4ccccc4-c4ccc(N(c5ccccc5)c5ccc(C6CCCCC6)cc5)cc42)c2ccccc2-3)c2ccc3ccccc3c2)cc1 ; c1ccc(-c2cc3ccccc3cc2N(c2ccc3c(c2)C2(c4ccccc4-c4ccc(N(c5ccccc5)c5ccc(C6CC7CCC6C7)cc5)cc42)c2ccccc2-3)c2ccc3ccccc3c2)cc1 ; c1ccc(-c2cc3ccccc3cc2N(c2ccc3c(c2)C2(c4ccccc4-c4ccc(N(c5ccccc5)c5ccc6ccccc6c5)cc42)c2ccccc2-3)c2ccc3ccccc3c2)cc1 ; c1ccc(-c2cc3ccccc3cc2N(c2ccc3c(c2)C2(c4ccccc4-c4ccc(N(c5ccc6ccccc6c5)c5ccc6ccccc6c5)cc42)c2ccccc2-3)c2ccc3ccccc3c2)cc1]

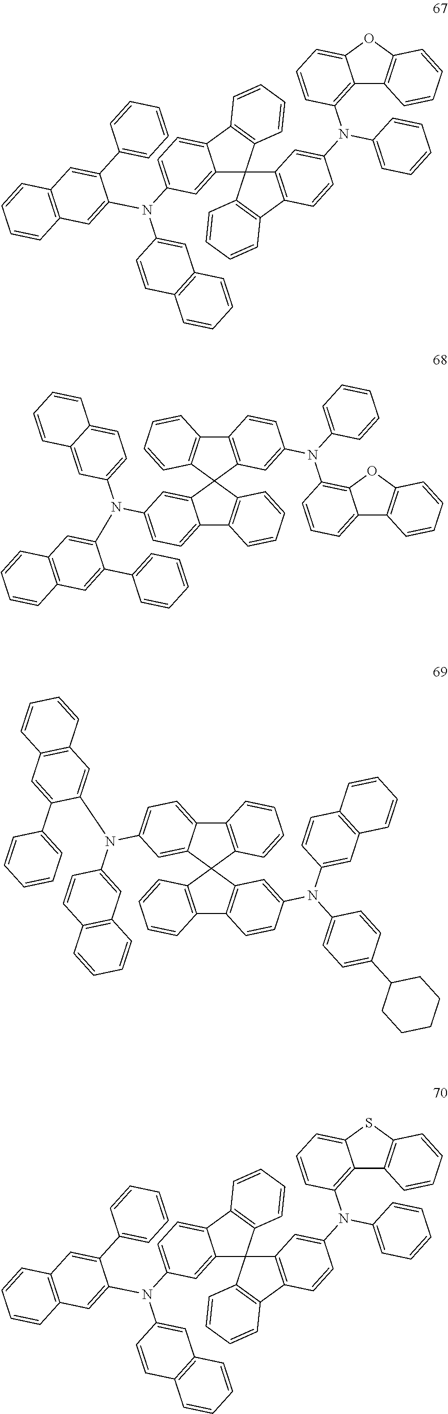 [SMILES: c1ccc(-c2cc3ccccc3cc2N(c2ccc3c(c2)C2(c4ccccc4-3)c3ccccc3-c3ccc(N(c4ccccc4)c4cccc5oc6ccccc6c45)cc32)c2ccc3ccccc3c2)cc1 ; c1ccc(-c2cc3ccccc3cc2N(c2ccc3c(c2)C2(c4ccccc4-3)c3ccccc3-c3ccc(N(c4ccccc4)c4cccc5c4oc4ccccc45)cc32)c2ccc3ccccc3c2)cc1 ; c1ccc(-c2cc3ccccc3cc2N(c2ccc3c(c2)C2(c4ccccc4-c4ccc(N(c5ccc(C6CCCCC6)cc5)c5ccc6ccccc6c5)cc42)c2ccccc2-3)c2ccc3ccccc3c2)cc1 ; c1ccc(-c2cc3ccccc3cc2N(c2ccc3c(c2)C2(c4ccccc4-3)c3ccccc3-c3ccc(N(c4ccccc4)c4cccc5sc6ccccc6c45)cc32)c2ccc3ccccc3c2)cc1]

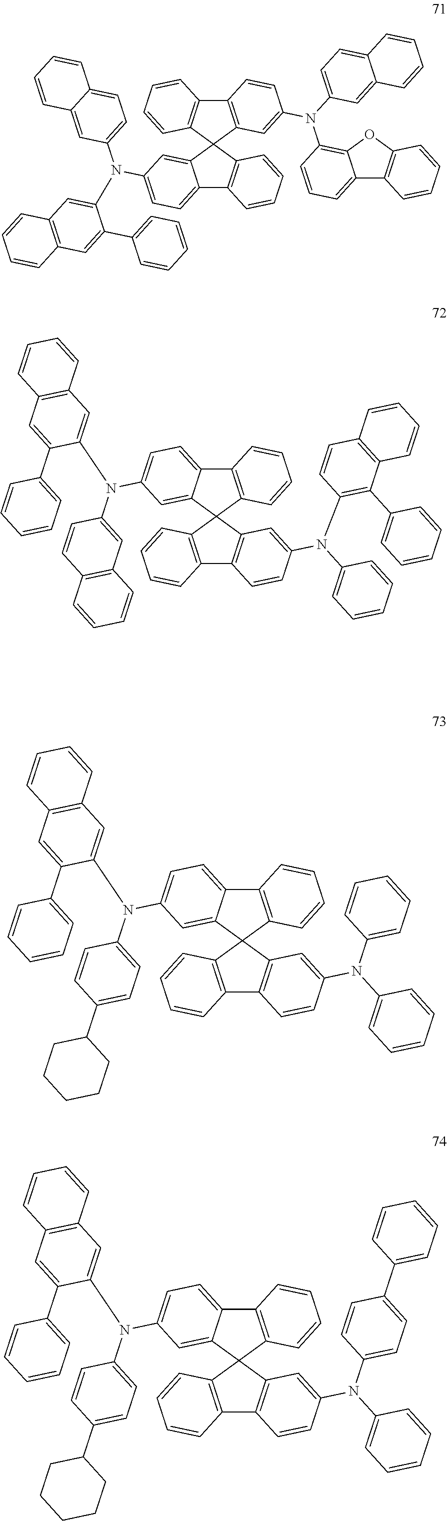 [SMILES: c1ccc(-c2cc3ccccc3cc2N(c2ccc3c(c2)C2(c4ccccc4-3)c3ccccc3-c3ccc(N(c4ccc5ccccc5c4)c4cccc5c4oc4ccccc45)cc32)c2ccc3ccccc3c2)cc1 ; c1ccc(-c2cc3ccccc3cc2N(c2ccc3c(c2)C2(c4ccccc4-3)c3ccccc3-c3ccc(N(c4ccccc4)c4ccc5ccccc5c4-c4ccccc4)cc32)c2ccc3ccccc3c2)cc1 ; c1ccc(-c2cc3ccccc3cc2N(c2ccc(C3CCCCC3)cc2)c2ccc3c(c2)C2(c4ccccc4-c4ccc(N(c5ccccc5)c5ccccc5)cc42)c2ccccc2-3)cc1 ; c1ccc(-c2ccc(N(c3ccccc3)c3ccc4c(c3)C3(c5ccccc5-4)c4ccccc4-c4ccc(N(c5ccc(C6CCCCC6)cc5)c5cc6ccccc6cc5-c5ccccc5)cc43)cc2)cc1]

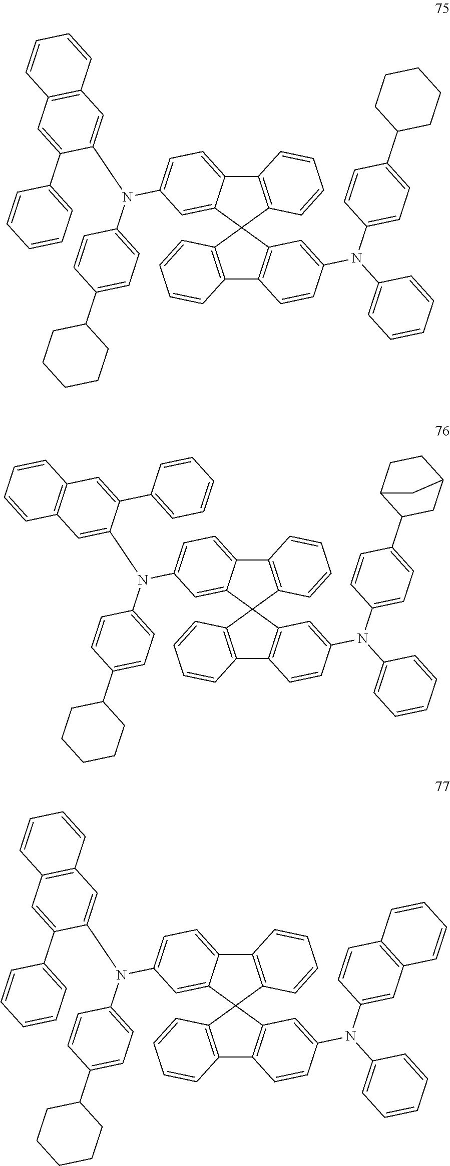 [SMILES: c1ccc(-c2cc3ccccc3cc2N(c2ccc(C3CCCCC3)cc2)c2ccc3c(c2)C2(c4ccccc4-c4ccc(N(c5ccccc5)c5ccc(C6CCCCC6)cc5)cc42)c2ccccc2-3)cc1 ; c1ccc(-c2cc3ccccc3cc2N(c2ccc(C3CCCCC3)cc2)c2ccc3c(c2)C2(c4ccccc4-c4ccc(N(c5ccccc5)c5ccc(C6CC7CCC6C7)cc5)cc42)c2ccccc2-3)cc1 ; c1ccc(-c2cc3ccccc3cc2N(c2ccc(C3CCCCC3)cc2)c2ccc3c(c2)C2(c4ccccc4-c4ccc(N(c5ccccc5)c5ccc6ccccc6c5)cc42)c2ccccc2-3)cc1]

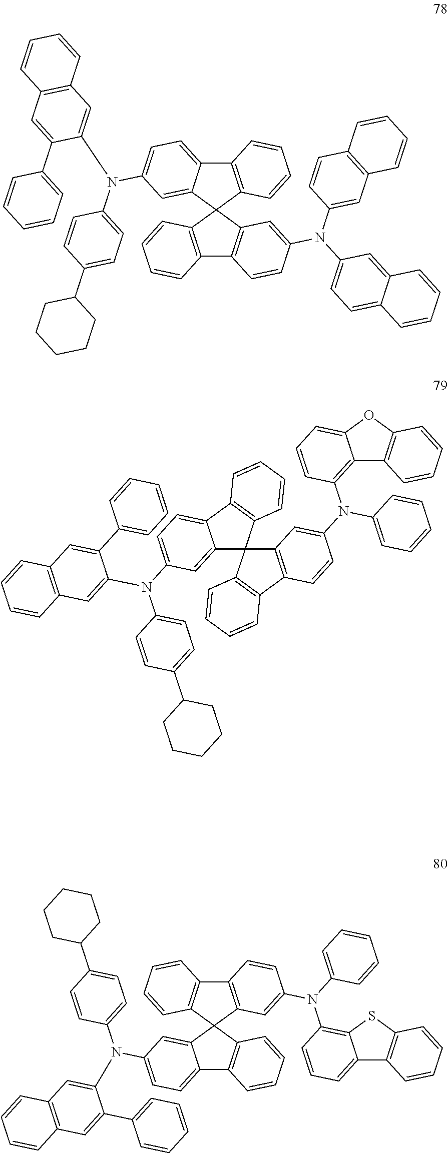 [SMILES: c1ccc(-c2cc3ccccc3cc2N(c2ccc(C3CCCCC3)cc2)c2ccc3c(c2)C2(c4ccccc4-c4ccc(N(c5ccc6ccccc6c5)c5ccc6ccccc6c5)cc42)c2ccccc2-3)cc1 ; c1ccc(-c2cc3ccccc3cc2N(c2ccc(C3CCCCC3)cc2)c2ccc3c(c2)C2(c4ccccc4-3)c3ccccc3-c3ccc(N(c4ccccc4)c4cccc5oc6ccccc6c45)cc32)cc1 ; c1ccc(-c2cc3ccccc3cc2N(c2ccc(C3CCCCC3)cc2)c2ccc3c(c2)C2(c4ccccc4-3)c3ccccc3-c3ccc(N(c4ccccc4)c4cccc5c4sc4ccccc45)cc32)cc1]

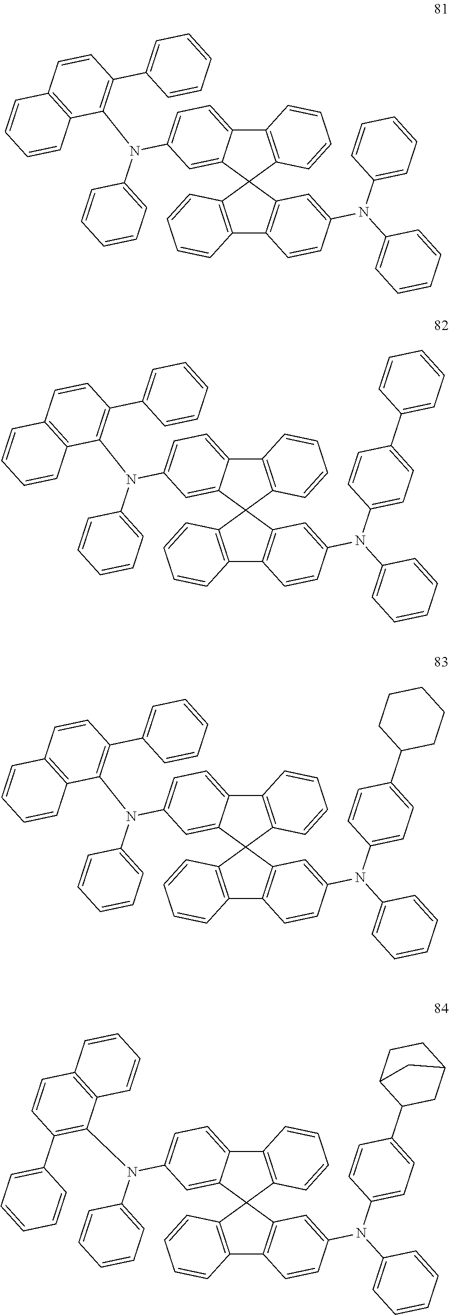 [SMILES: c1ccc(-c2ccc3ccccc3c2N(c2ccccc2)c2ccc3c(c2)C2(c4ccccc4-c4ccc(N(c5ccccc5)c5ccccc5)cc42)c2ccccc2-3)cc1 ; c1ccc(-c2ccc(N(c3ccccc3)c3ccc4c(c3)C3(c5ccccc5-4)c4ccccc4-c4ccc(N(c5ccccc5)c5c(-c6ccccc6)ccc6ccccc56)cc43)cc2)cc1 ; c1ccc(-c2ccc3ccccc3c2N(c2ccccc2)c2ccc3c(c2)C2(c4ccccc4-c4ccc(N(c5ccccc5)c5ccc(C6CCCCC6)cc5)cc42)c2ccccc2-3)cc1 ; c1ccc(-c2ccc3ccccc3c2N(c2ccccc2)c2ccc3c(c2)C2(c4ccccc4-c4ccc(N(c5ccccc5)c5ccc(C6CC7CCC6C7)cc5)cc42)c2ccccc2-3)cc1]

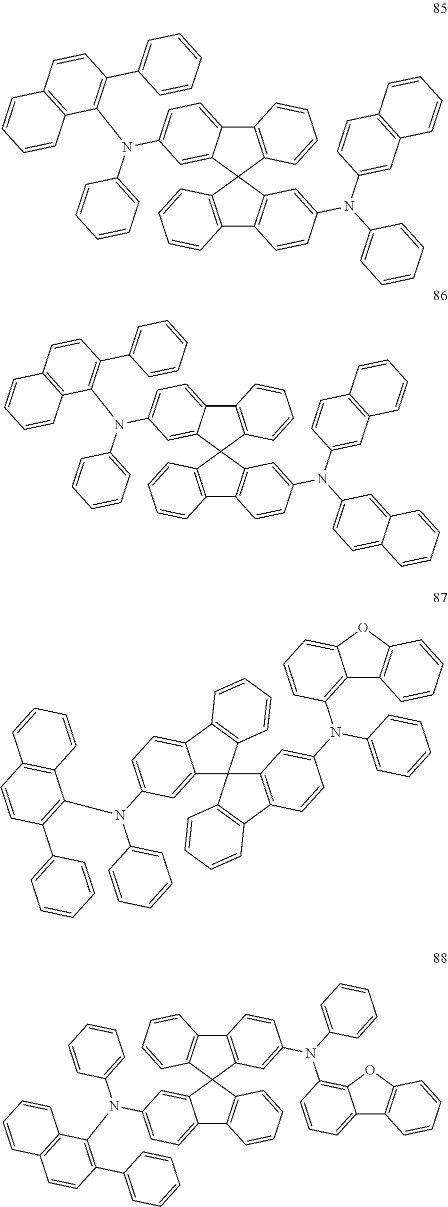 [SMILES: c1ccc(-c2ccc3ccccc3c2N(c2ccccc2)c2ccc3c(c2)C2(c4ccccc4-c4ccc(N(c5ccccc5)c5ccc6ccccc6c5)cc42)c2ccccc2-3)cc1 ; c1ccc(-c2ccc3ccccc3c2N(c2ccccc2)c2ccc3c(c2)C2(c4ccccc4-c4ccc(N(c5ccc6ccccc6c5)c5ccc6ccccc6c5)cc42)c2ccccc2-3)cc1 ; c1ccc(-c2ccc3ccccc3c2N(c2ccccc2)c2ccc3c(c2)C2(c4ccccc4-3)c3ccccc3-c3ccc(N(c4ccccc4)c4cccc5oc6ccccc6c45)cc32)cc1 ; c1ccc(-c2ccc3ccccc3c2N(c2ccccc2)c2ccc3c(c2)C2(c4ccccc4-3)c3ccccc3-c3ccc(N(c4ccccc4)c4cccc5c4oc4ccccc45)cc32)cc1]

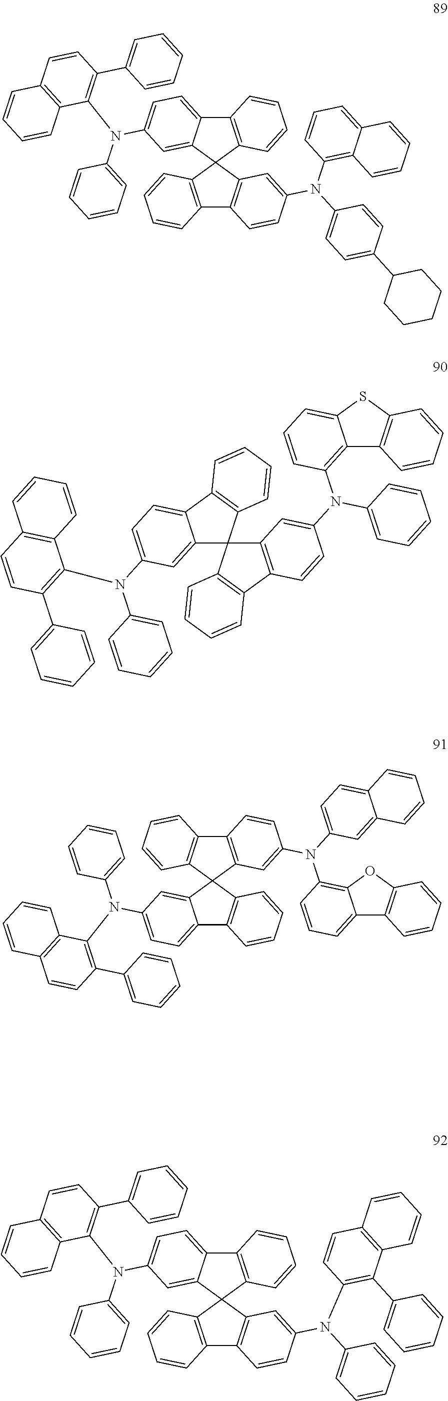 [SMILES: c1ccc(-c2ccc3ccccc3c2N(c2ccccc2)c2ccc3c(c2)C2(c4ccccc4-c4ccc(N(c5ccc(C6CCCCC6)cc5)c5cccc6ccccc56)cc42)c2ccccc2-3)cc1 ; c1ccc(-c2ccc3ccccc3c2N(c2ccccc2)c2ccc3c(c2)C2(c4ccccc4-3)c3ccccc3-c3ccc(N(c4ccccc4)c4cccc5sc6ccccc6c45)cc32)cc1 ; c1ccc(-c2ccc3ccccc3c2N(c2ccccc2)c2ccc3c(c2)C2(c4ccccc4-3)c3ccccc3-c3ccc(N(c4ccc5ccccc5c4)c4cccc5c4oc4ccccc45)cc32)cc1 ; c1ccc(-c2ccc3ccccc3c2N(c2ccccc2)c2ccc3c(c2)C2(c4ccccc4-c4ccc(N(c5ccccc5)c5ccc6ccccc6c5-c5ccccc5)cc42)c2ccccc2-3)cc1]

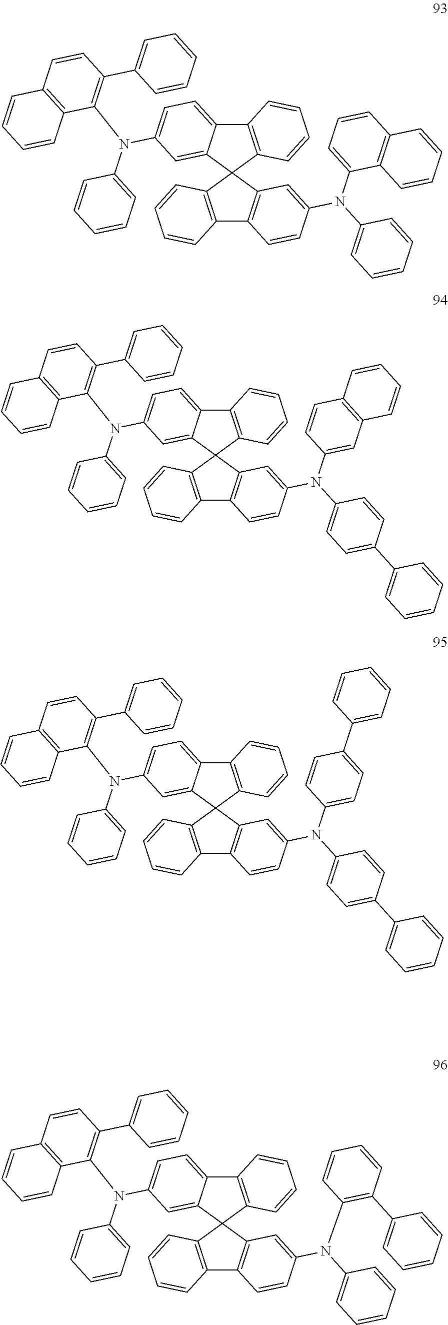 [SMILES: c1ccc(-c2ccc3ccccc3c2N(c2ccccc2)c2ccc3c(c2)C2(c4ccccc4-c4ccc(N(c5ccccc5)c5cccc6ccccc56)cc42)c2ccccc2-3)cc1 ; c1ccc(-c2ccc(N(c3ccc4c(c3)C3(c5ccccc5-4)c4ccccc4-c4ccc(N(c5ccccc5)c5c(-c6ccccc6)ccc6ccccc56)cc43)c3ccc4ccccc4c3)cc2)cc1 ; c1ccc(-c2ccc(N(c3ccc(-c4ccccc4)cc3)c3ccc4c(c3)C3(c5ccccc5-4)c4ccccc4-c4ccc(N(c5ccccc5)c5c(-c6ccccc6)ccc6ccccc56)cc43)cc2)cc1 ; c1ccc(-c2ccccc2N(c2ccccc2)c2ccc3c(c2)C2(c4ccccc4-3)c3ccccc3-c3ccc(N(c4ccccc4)c4c(-c5ccccc5)ccc5ccccc45)cc32)cc1]

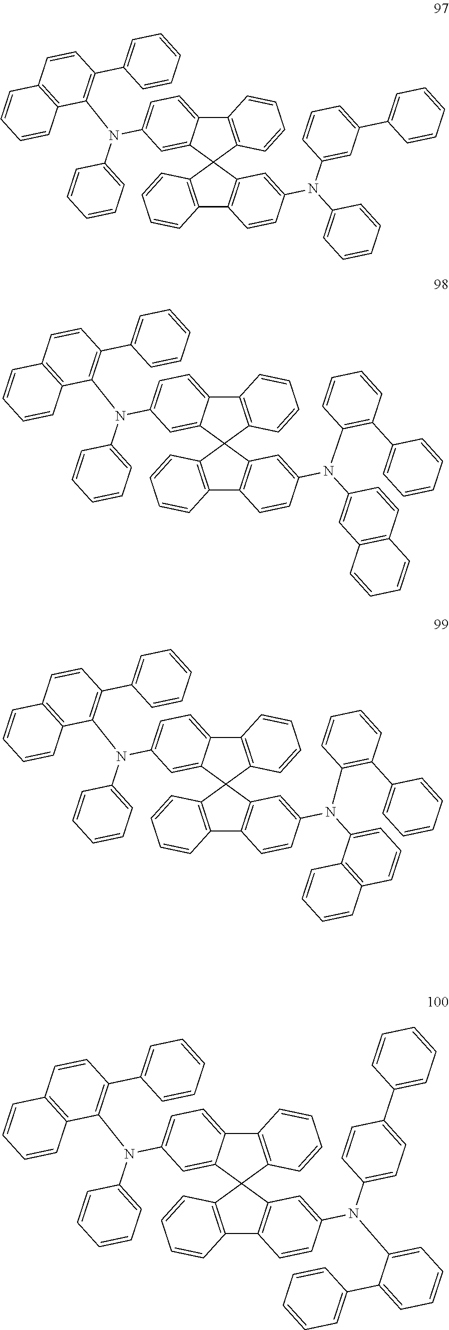 [SMILES: c1ccc(-c2cccc(N(c3ccccc3)c3ccc4c(c3)C3(c5ccccc5-4)c4ccccc4-c4ccc(N(c5ccccc5)c5c(-c6ccccc6)ccc6ccccc56)cc43)c2)cc1 ; c1ccc(-c2ccccc2N(c2ccc3c(c2)C2(c4ccccc4-3)c3ccccc3-c3ccc(N(c4ccccc4)c4c(-c5ccccc5)ccc5ccccc45)cc32)c2ccc3ccccc3c2)cc1 ; c1ccc(-c2ccccc2N(c2ccc3c(c2)C2(c4ccccc4-3)c3ccccc3-c3ccc(N(c4ccccc4)c4c(-c5ccccc5)ccc5ccccc45)cc32)c2cccc3ccccc23)cc1 ; c1ccc(-c2ccc(N(c3ccc4c(c3)C3(c5ccccc5-4)c4ccccc4-c4ccc(N(c5ccccc5)c5c(-c6ccccc6)ccc6ccccc56)cc43)c3ccccc3-c3ccccc3)cc2)cc1]

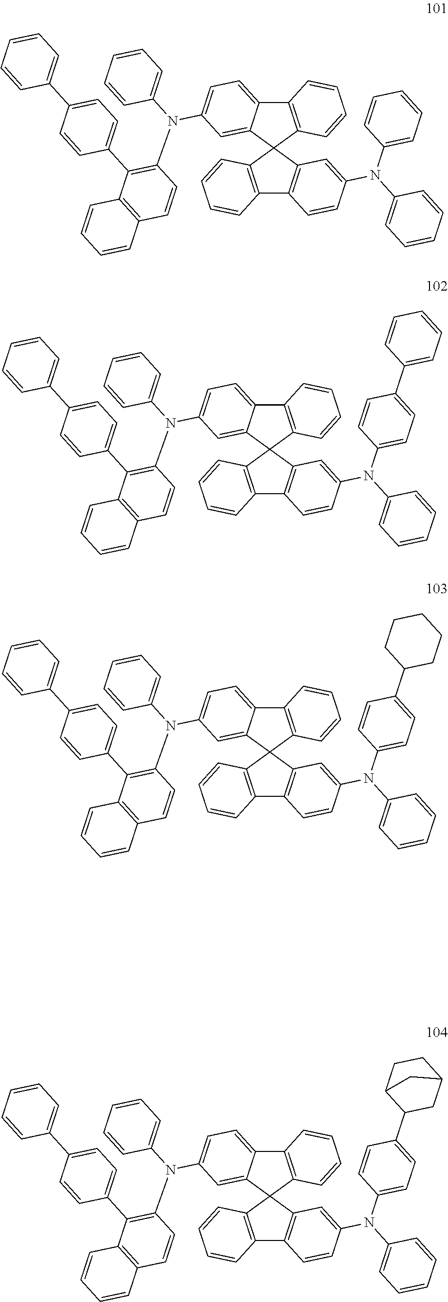 [SMILES: c1ccc(-c2ccc(-c3c(N(c4ccccc4)c4ccc5c(c4)C4(c6ccccc6-c6ccc(N(c7ccccc7)c7ccccc7)cc64)c4ccccc4-5)ccc4ccccc34)cc2)cc1 ; c1ccc(-c2ccc(-c3c(N(c4ccccc4)c4ccc5c(c4)C4(c6ccccc6-c6ccc(N(c7ccccc7)c7ccc(-c8ccccc8)cc7)cc64)c4ccccc4-5)ccc4ccccc34)cc2)cc1 ; c1ccc(-c2ccc(-c3c(N(c4ccccc4)c4ccc5c(c4)C4(c6ccccc6-c6ccc(N(c7ccccc7)c7ccc(C8CCCCC8)cc7)cc64)c4ccccc4-5)ccc4ccccc34)cc2)cc1 ; c1ccc(-c2ccc(-c3c(N(c4ccccc4)c4ccc5c(c4)C4(c6ccccc6-c6ccc(N(c7ccccc7)c7ccc(C8CC9CCC8C9)cc7)cc64)c4ccccc4-5)ccc4ccccc34)cc2)cc1]

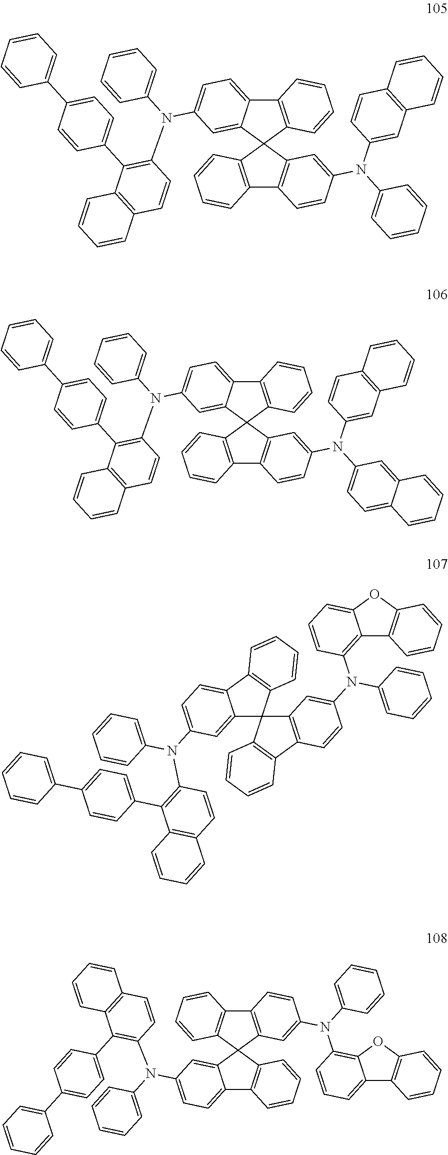

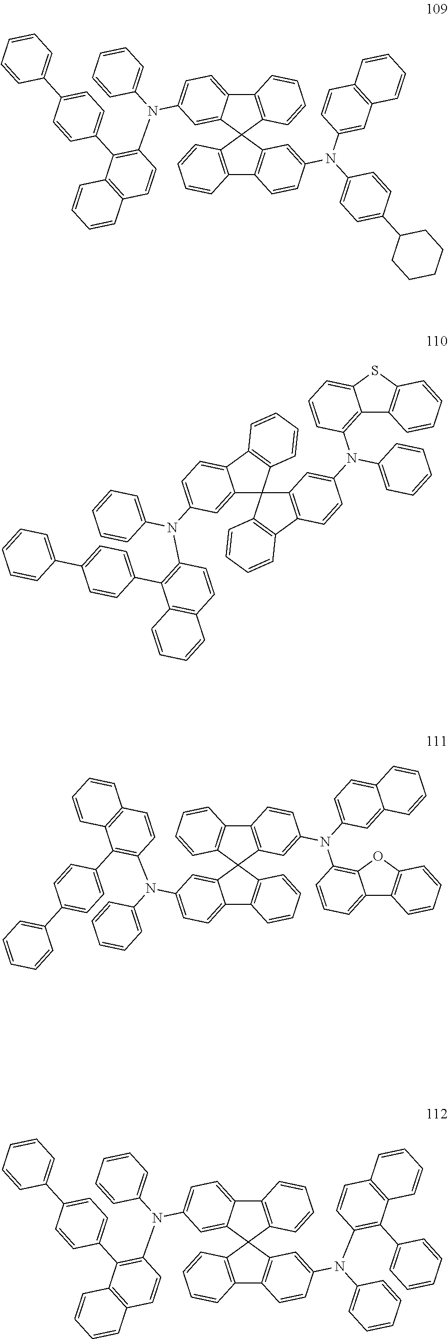

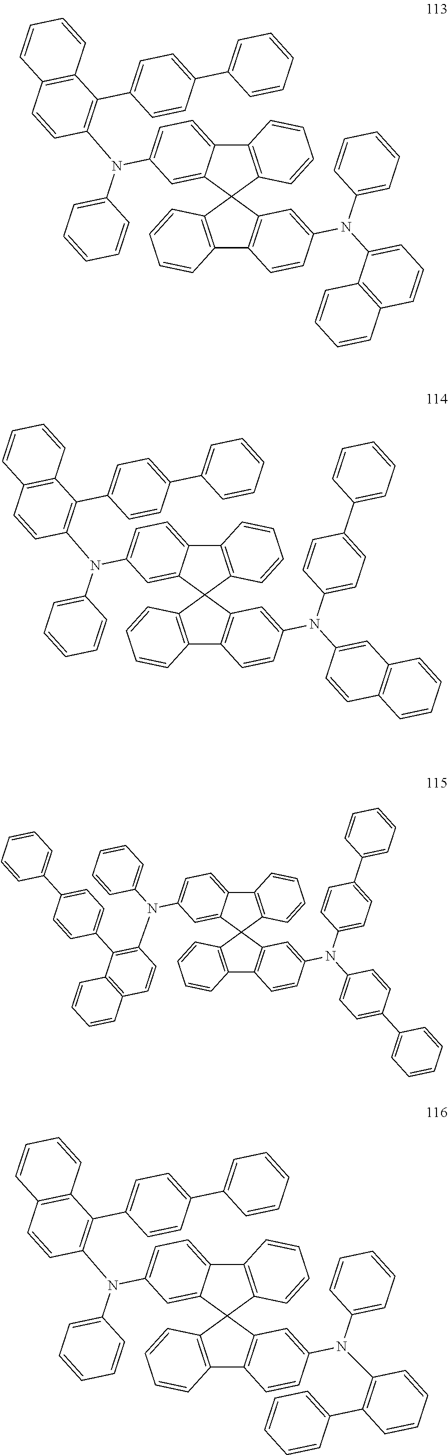

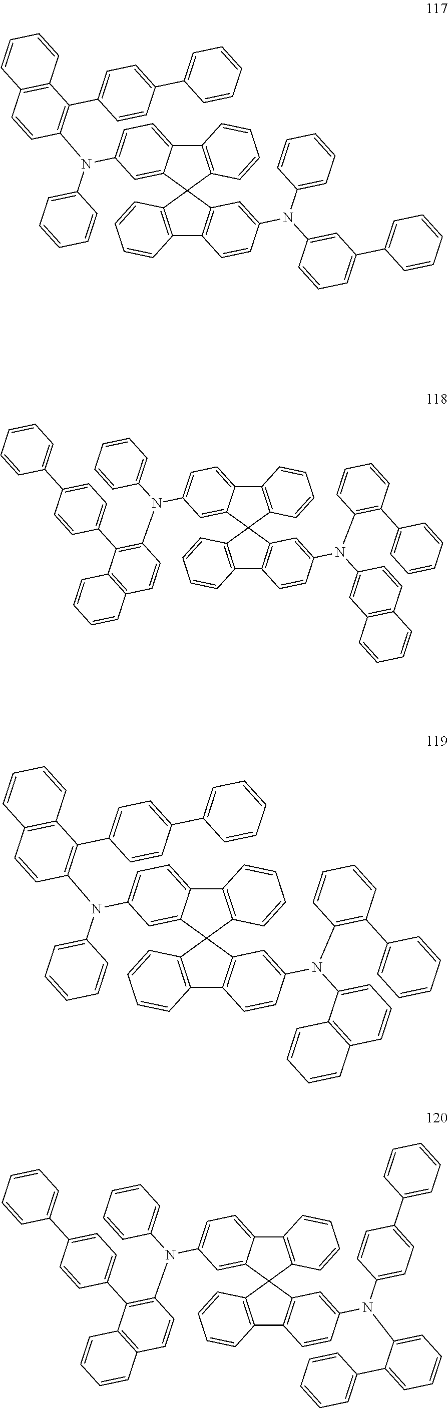

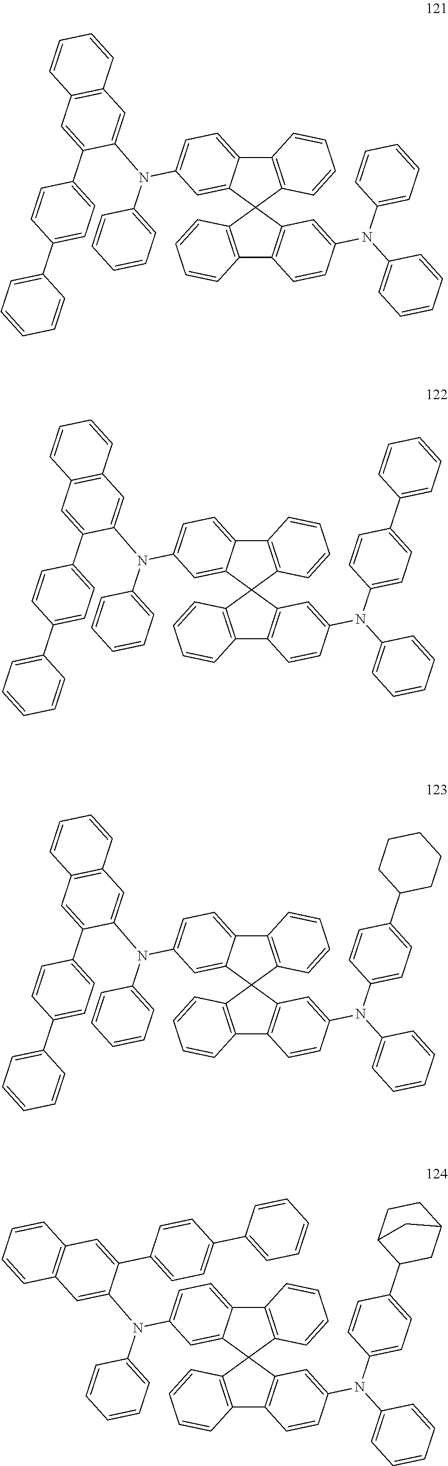 [SMILES: c1ccc(-c2ccc(-c3cc4ccccc4cc3N(c3ccccc3)c3ccc4c(c3)C3(c5ccccc5-c5ccc(N(c6ccccc6)c6ccccc6)cc53)c3ccccc3-4)cc2)cc1 ; c1ccc(-c2ccc(-c3cc4ccccc4cc3N(c3ccccc3)c3ccc4c(c3)C3(c5ccccc5-c5ccc(N(c6ccccc6)c6ccc(-c7ccccc7)cc6)cc53)c3ccccc3-4)cc2)cc1 ; c1ccc(-c2ccc(-c3cc4ccccc4cc3N(c3ccccc3)c3ccc4c(c3)C3(c5ccccc5-c5ccc(N(c6ccccc6)c6ccc(C7CCCCC7)cc6)cc53)c3ccccc3-4)cc2)cc1 ; c1ccc(-c2ccc(-c3cc4ccccc4cc3N(c3ccccc3)c3ccc4c(c3)C3(c5ccccc5-c5ccc(N(c6ccccc6)c6ccc(C7CC8CCC7C8)cc6)cc53)c3ccccc3-4)cc2)cc1]

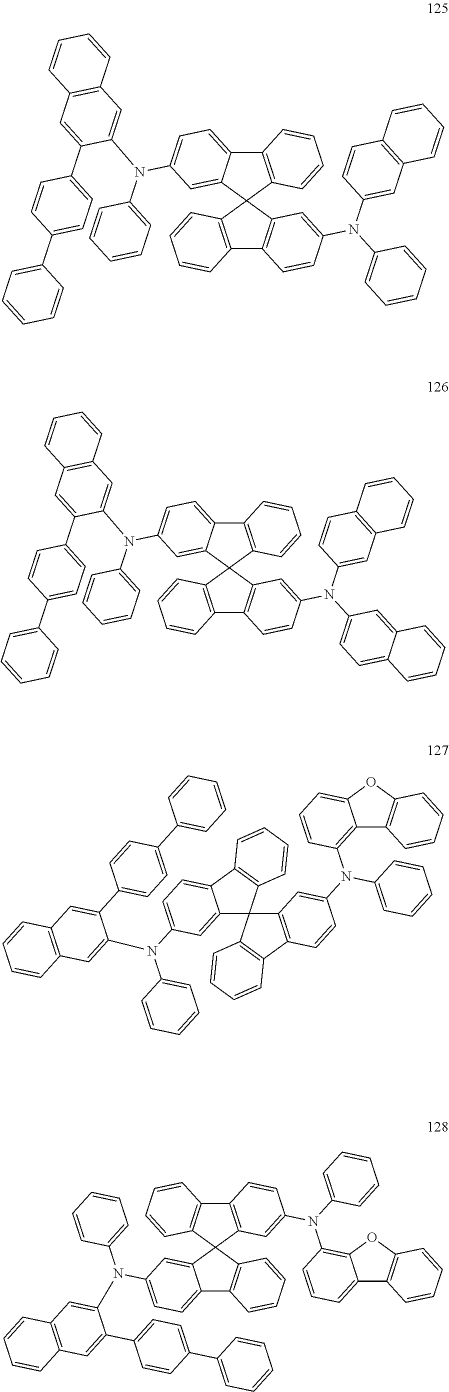 [SMILES: c1ccc(-c2ccc(-c3cc4ccccc4cc3N(c3ccccc3)c3ccc4c(c3)C3(c5ccccc5-c5ccc(N(c6ccccc6)c6ccc7ccccc7c6)cc53)c3ccccc3-4)cc2)cc1 ; c1ccc(-c2ccc(-c3cc4ccccc4cc3N(c3ccccc3)c3ccc4c(c3)C3(c5ccccc5-c5ccc(N(c6ccc7ccccc7c6)c6ccc7ccccc7c6)cc53)c3ccccc3-4)cc2)cc1 ; c1ccc(-c2ccc(-c3cc4ccccc4cc3N(c3ccccc3)c3ccc4c(c3)C3(c5ccccc5-4)c4ccccc4-c4ccc(N(c5ccccc5)c5cccc6oc7ccccc7c56)cc43)cc2)cc1 ; c1ccc(-c2ccc(-c3cc4ccccc4cc3N(c3ccccc3)c3ccc4c(c3)C3(c5ccccc5-4)c4ccccc4-c4ccc(N(c5ccccc5)c5cccc6c5oc5ccccc56)cc43)cc2)cc1]

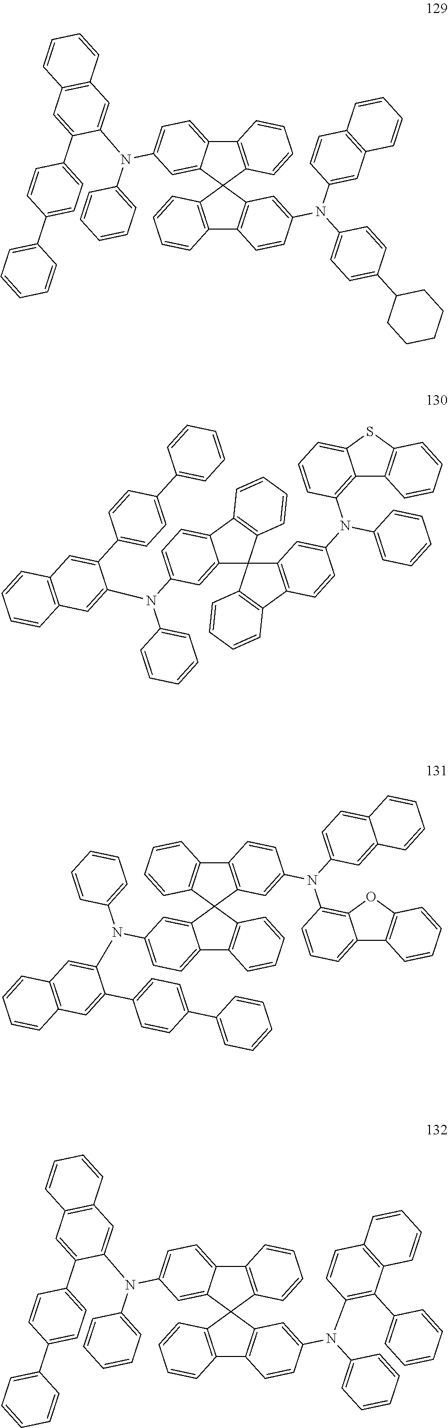

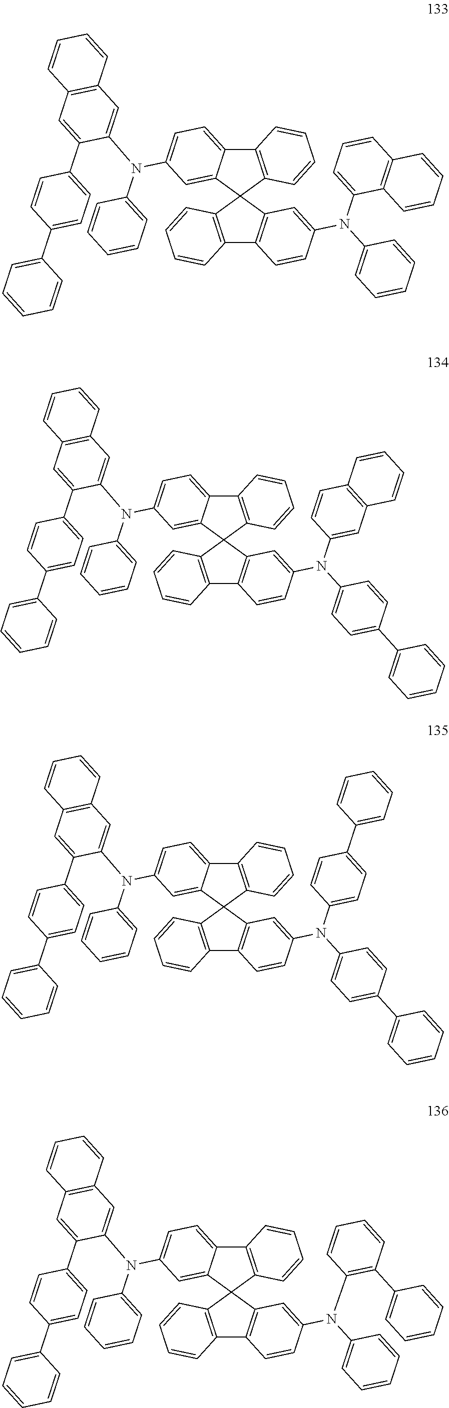

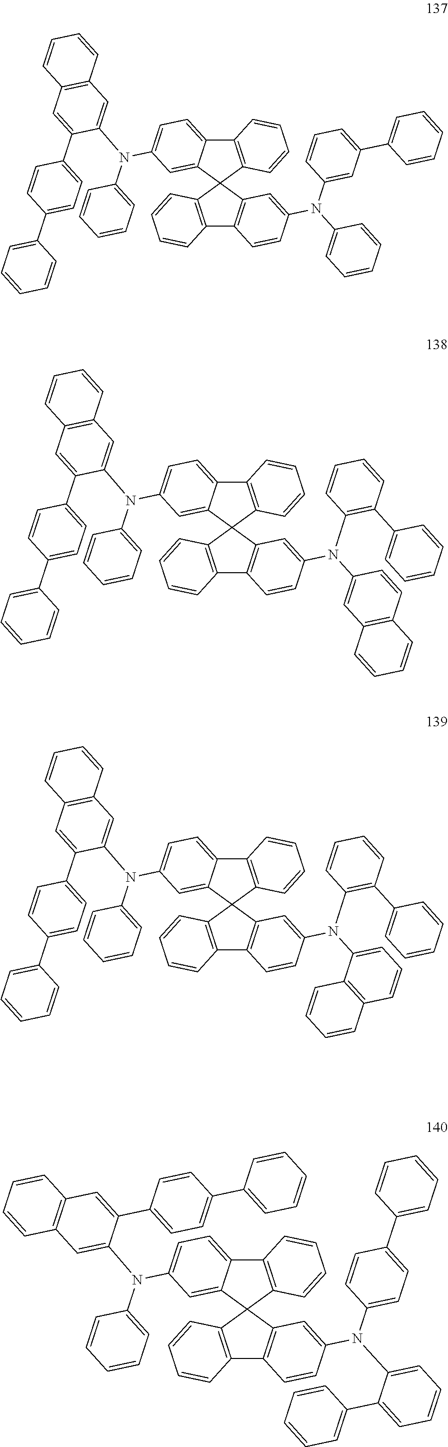

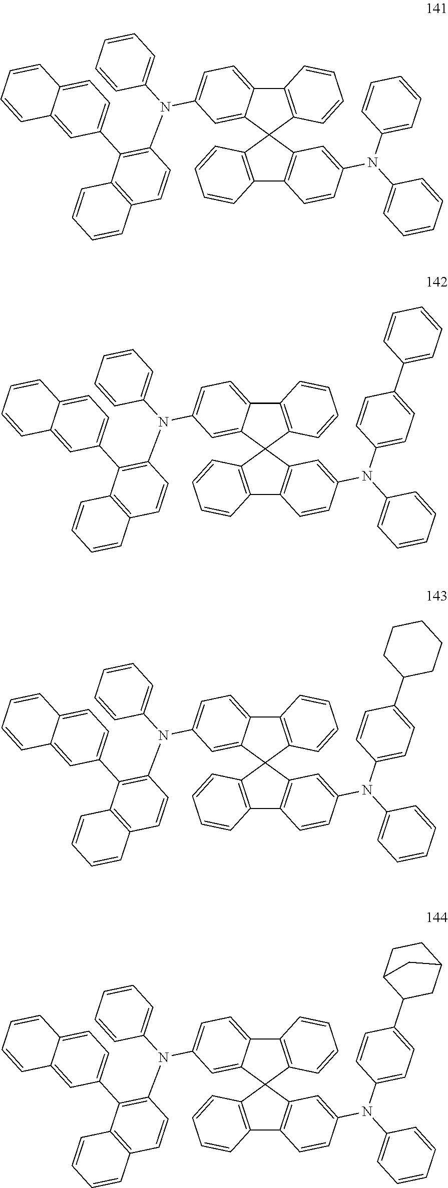 [SMILES: c1ccc(N(c2ccccc2)c2ccc3c(c2)C2(c4ccccc4-3)c3ccccc3-c3ccc(N(c4ccccc4)c4ccc5ccccc5c4-c4ccc5ccccc5c4)cc32)cc1 ; c1ccc(-c2ccc(N(c3ccccc3)c3ccc4c(c3)C3(c5ccccc5-4)c4ccccc4-c4ccc(N(c5ccccc5)c5ccc6ccccc6c5-c5ccc6ccccc6c5)cc43)cc2)cc1 ; c1ccc(N(c2ccc(C3CCCCC3)cc2)c2ccc3c(c2)C2(c4ccccc4-3)c3ccccc3-c3ccc(N(c4ccccc4)c4ccc5ccccc5c4-c4ccc5ccccc5c4)cc32)cc1 ; c1ccc(N(c2ccc(C3CC4CCC3C4)cc2)c2ccc3c(c2)C2(c4ccccc4-3)c3ccccc3-c3ccc(N(c4ccccc4)c4ccc5ccccc5c4-c4ccc5ccccc5c4)cc32)cc1]

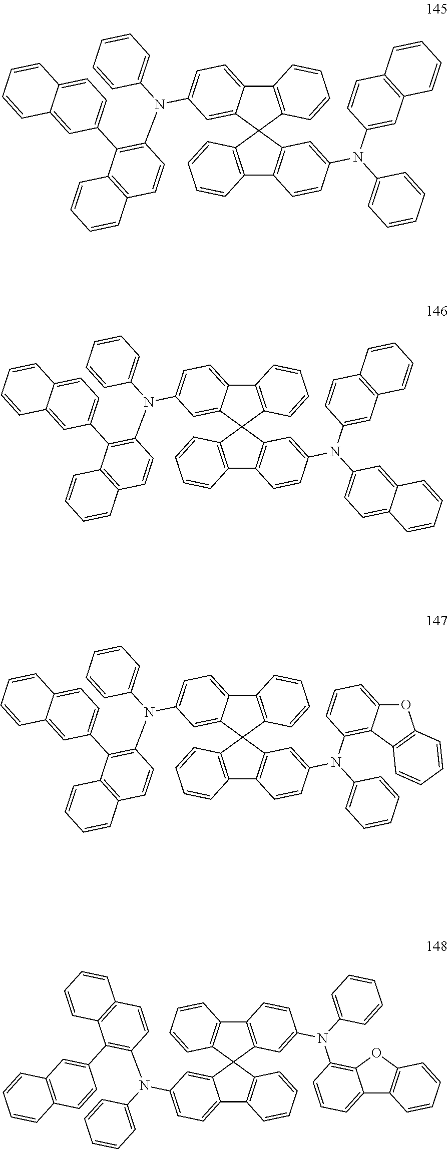 [SMILES: c1ccc(N(c2ccc3c(c2)C2(c4ccccc4-3)c3ccccc3-c3ccc(N(c4ccccc4)c4ccc5ccccc5c4-c4ccc5ccccc5c4)cc32)c2ccc3ccccc3c2)cc1 ; c1ccc(N(c2ccc3c(c2)C2(c4ccccc4-c4ccc(N(c5ccc6ccccc6c5)c5ccc6ccccc6c5)cc42)c2ccccc2-3)c2ccc3ccccc3c2-c2ccc3ccccc3c2)cc1 ; c1ccc(N(c2ccc3c(c2)C2(c4ccccc4-3)c3ccccc3-c3ccc(N(c4ccccc4)c4cccc5oc6ccccc6c45)cc32)c2ccc3ccccc3c2-c2ccc3ccccc3c2)cc1 ; c1ccc(N(c2ccc3c(c2)C2(c4ccccc4-3)c3ccccc3-c3ccc(N(c4ccccc4)c4cccc5c4oc4ccccc45)cc32)c2ccc3ccccc3c2-c2ccc3ccccc3c2)cc1]

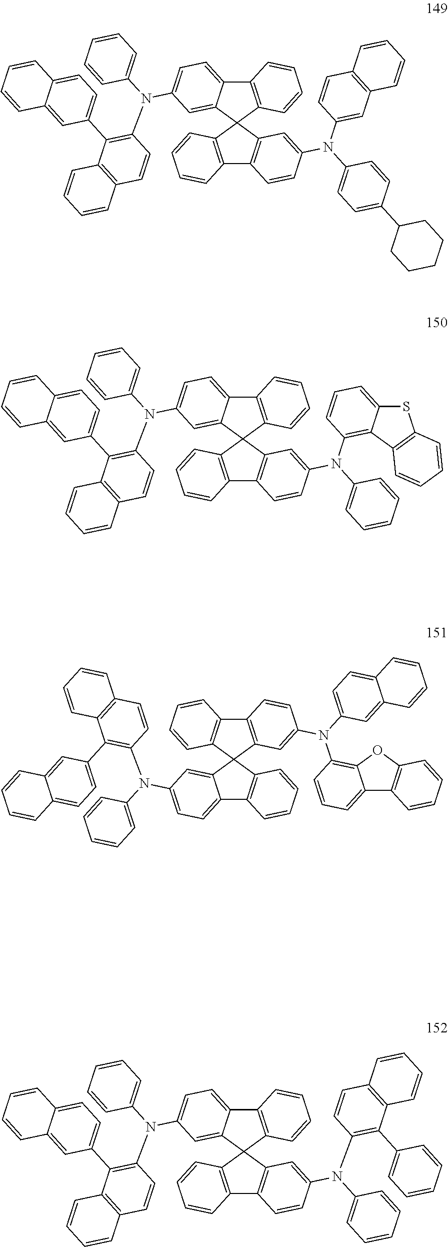 [SMILES: c1ccc(N(c2ccc3c(c2)C2(c4ccccc4-c4ccc(N(c5ccc(C6CCCCC6)cc5)c5ccc6ccccc6c5)cc42)c2ccccc2-3)c2ccc3ccccc3c2-c2ccc3ccccc3c2)cc1 ; c1ccc(N(c2ccc3c(c2)C2(c4ccccc4-3)c3ccccc3-c3ccc(N(c4ccccc4)c4cccc5sc6ccccc6c45)cc32)c2ccc3ccccc3c2-c2ccc3ccccc3c2)cc1 ; c1ccc(N(c2ccc3c(c2)C2(c4ccccc4-3)c3ccccc3-c3ccc(N(c4ccc5ccccc5c4)c4cccc5c4oc4ccccc45)cc32)c2ccc3ccccc3c2-c2ccc3ccccc3c2)cc1 ; c1ccc(-c2c(N(c3ccccc3)c3ccc4c(c3)C3(c5ccccc5-4)c4ccccc4-c4ccc(N(c5ccccc5)c5ccc6ccccc6c5-c5ccc6ccccc6c5)cc43)ccc3ccccc23)cc1]

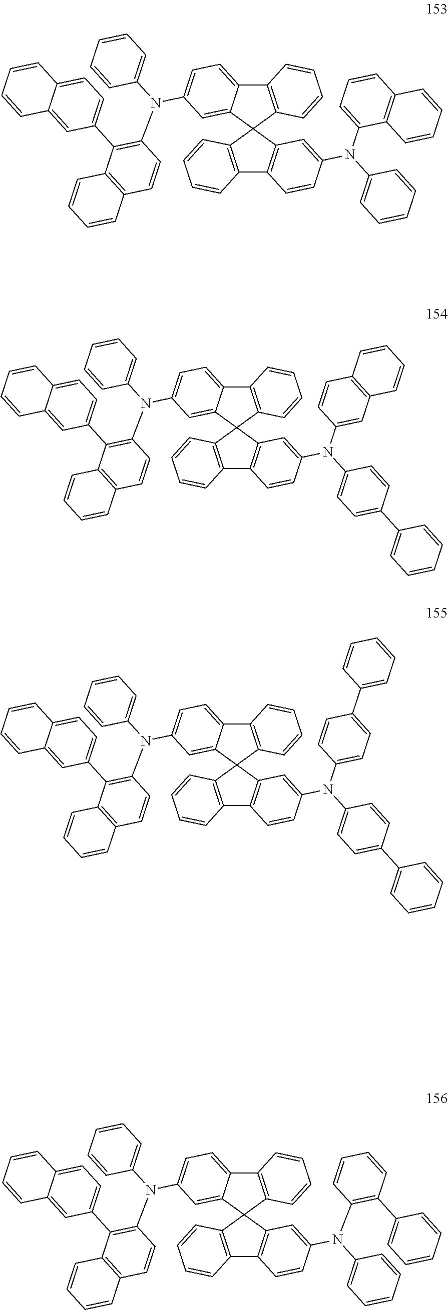 [SMILES: c1ccc(N(c2ccc3c(c2)C2(c4ccccc4-3)c3ccccc3-c3ccc(N(c4ccccc4)c4cccc5ccccc45)cc32)c2ccc3ccccc3c2-c2ccc3ccccc3c2)cc1 ; c1ccc(-c2ccc(N(c3ccc4c(c3)C3(c5ccccc5-4)c4ccccc4-c4ccc(N(c5ccccc5)c5ccc6ccccc6c5-c5ccc6ccccc6c5)cc43)c3ccc4ccccc4c3)cc2)cc1 ; c1ccc(-c2ccc(N(c3ccc(-c4ccccc4)cc3)c3ccc4c(c3)C3(c5ccccc5-4)c4ccccc4-c4ccc(N(c5ccccc5)c5ccc6ccccc6c5-c5ccc6ccccc6c5)cc43)cc2)cc1 ; c1ccc(-c2ccccc2N(c2ccccc2)c2ccc3c(c2)C2(c4ccccc4-3)c3ccccc3-c3ccc(N(c4ccccc4)c4ccc5ccccc5c4-c4ccc5ccccc5c4)cc32)cc1]

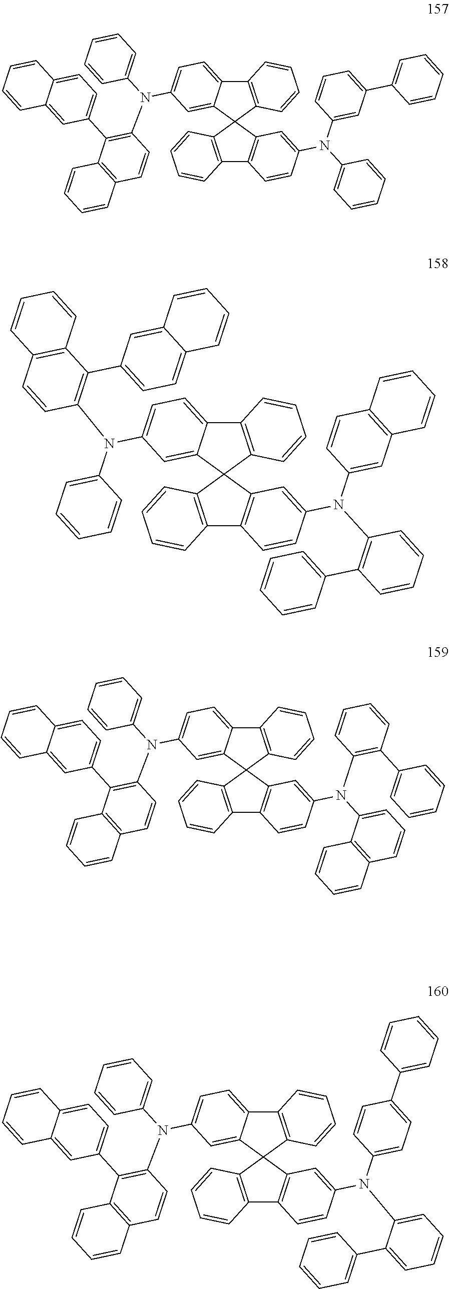 [SMILES: c1ccc(-c2cccc(N(c3ccccc3)c3ccc4c(c3)C3(c5ccccc5-4)c4ccccc4-c4ccc(N(c5ccccc5)c5ccc6ccccc6c5-c5ccc6ccccc6c5)cc43)c2)cc1 ; c1ccc(-c2ccccc2N(c2ccc3c(c2)C2(c4ccccc4-3)c3ccccc3-c3ccc(N(c4ccccc4)c4ccc5ccccc5c4-c4ccc5ccccc5c4)cc32)c2ccc3ccccc3c2)cc1 ; c1ccc(-c2ccccc2N(c2ccc3c(c2)C2(c4ccccc4-c4ccc(N(c5ccccc5)c5ccc6ccccc6c5-c5ccc6ccccc6c5)cc42)c2ccccc2-3)c2cccc3ccccc23)cc1 ; c1ccc(-c2ccc(N(c3ccc4c(c3)C3(c5ccccc5-4)c4ccccc4-c4ccc(N(c5ccccc5)c5ccc6ccccc6c5-c5ccc6ccccc6c5)cc43)c3ccccc3-c3ccccc3)cc2)cc1]

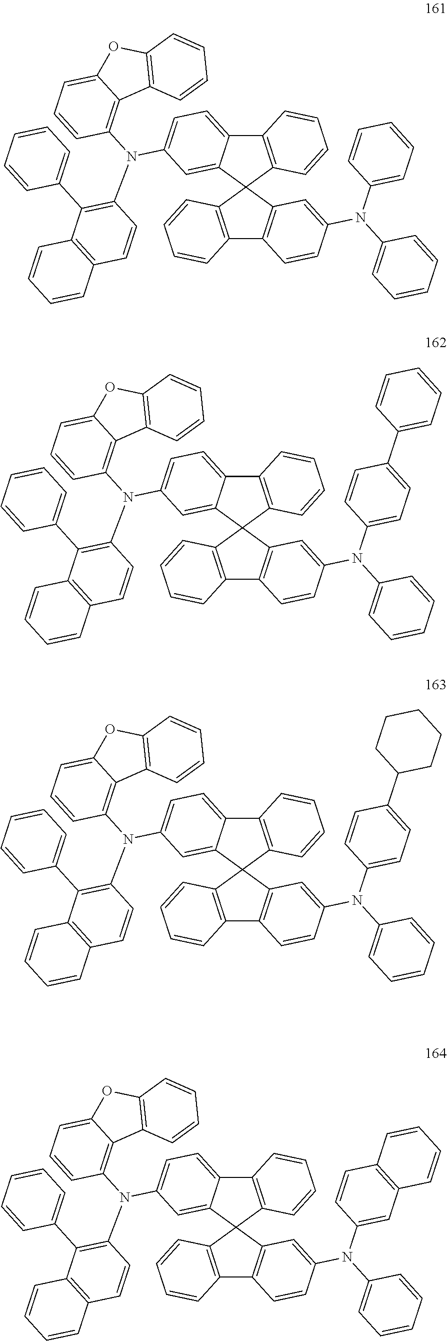 [SMILES: c1ccc(-c2c(N(c3ccc4c(c3)C3(c5ccccc5-c5ccc(N(c6ccccc6)c6ccccc6)cc53)c3ccccc3-4)c3cccc4oc5ccccc5c34)ccc3ccccc23)cc1 ; c1ccc(-c2ccc(N(c3ccccc3)c3ccc4c(c3)C3(c5ccccc5-4)c4ccccc4-c4ccc(N(c5ccc6ccccc6c5-c5ccccc5)c5cccc6oc7ccccc7c56)cc43)cc2)cc1 ; c1ccc(-c2c(N(c3ccc4c(c3)C3(c5ccccc5-c5ccc(N(c6ccccc6)c6ccc(C7CCCCC7)cc6)cc53)c3ccccc3-4)c3cccc4oc5ccccc5c34)ccc3ccccc23)cc1 ; c1ccc(-c2c(N(c3ccc4c(c3)C3(c5ccccc5-c5ccc(N(c6ccccc6)c6ccc7ccccc7c6)cc53)c3ccccc3-4)c3cccc4oc5ccccc5c34)ccc3ccccc23)cc1]

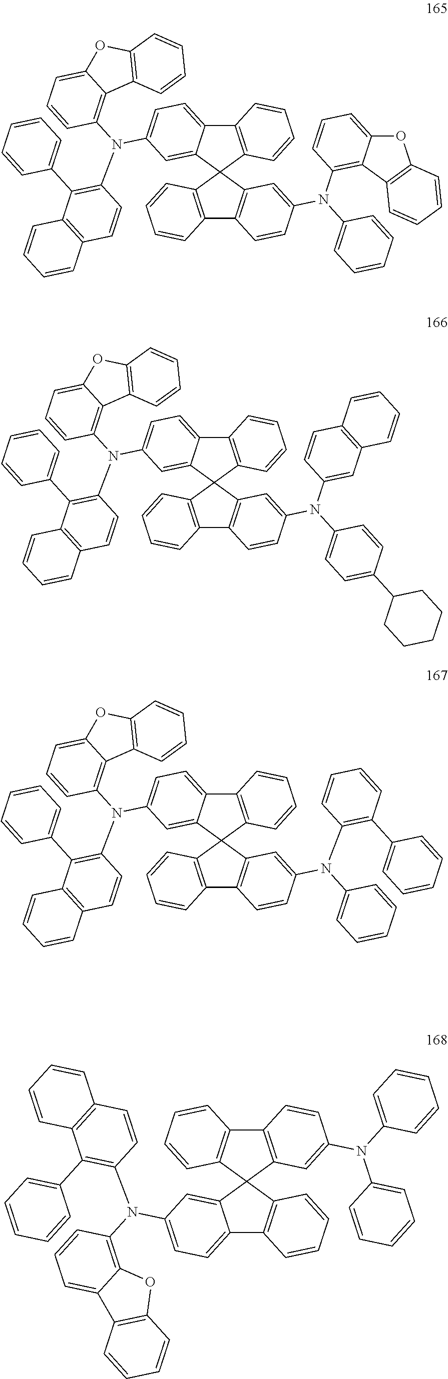 [SMILES: c1ccc(-c2c(N(c3ccc4c(c3)C3(c5ccccc5-c5ccc(N(c6ccccc6)c6cccc7oc8ccccc8c67)cc53)c3ccccc3-4)c3cccc4oc5ccccc5c34)ccc3ccccc23)cc1 ; c1ccc(-c2c(N(c3ccc4c(c3)C3(c5ccccc5-c5ccc(N(c6ccc(C7CCCCC7)cc6)c6ccc7ccccc7c6)cc53)c3ccccc3-4)c3cccc4oc5ccccc5c34)ccc3ccccc23)cc1 ; c1ccc(-c2ccccc2N(c2ccccc2)c2ccc3c(c2)C2(c4ccccc4-3)c3ccccc3-c3ccc(N(c4ccc5ccccc5c4-c4ccccc4)c4cccc5oc6ccccc6c45)cc32)cc1 ; c1ccc(-c2c(N(c3ccc4c(c3)C3(c5ccccc5-c5ccc(N(c6ccccc6)c6ccccc6)cc53)c3ccccc3-4)c3cccc4c3oc3ccccc34)ccc3ccccc23)cc1]

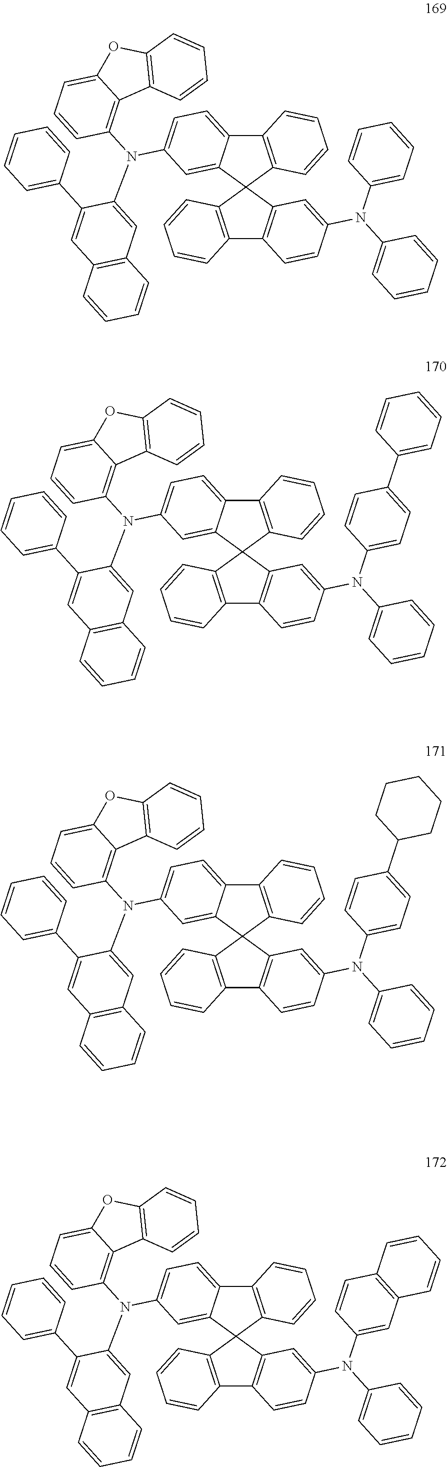 [SMILES: c1ccc(-c2cc3ccccc3cc2N(c2ccc3c(c2)C2(c4ccccc4-c4ccc(N(c5ccccc5)c5ccccc5)cc42)c2ccccc2-3)c2cccc3oc4ccccc4c23)cc1 ; c1ccc(-c2ccc(N(c3ccccc3)c3ccc4c(c3)C3(c5ccccc5-4)c4ccccc4-c4ccc(N(c5cc6ccccc6cc5-c5ccccc5)c5cccc6oc7ccccc7c56)cc43)cc2)cc1 ; c1ccc(-c2cc3ccccc3cc2N(c2ccc3c(c2)C2(c4ccccc4-c4ccc(N(c5ccccc5)c5ccc(C6CCCCC6)cc5)cc42)c2ccccc2-3)c2cccc3oc4ccccc4c23)cc1 ; c1ccc(-c2cc3ccccc3cc2N(c2ccc3c(c2)C2(c4ccccc4-c4ccc(N(c5ccccc5)c5ccc6ccccc6c5)cc42)c2ccccc2-3)c2cccc3oc4ccccc4c23)cc1]

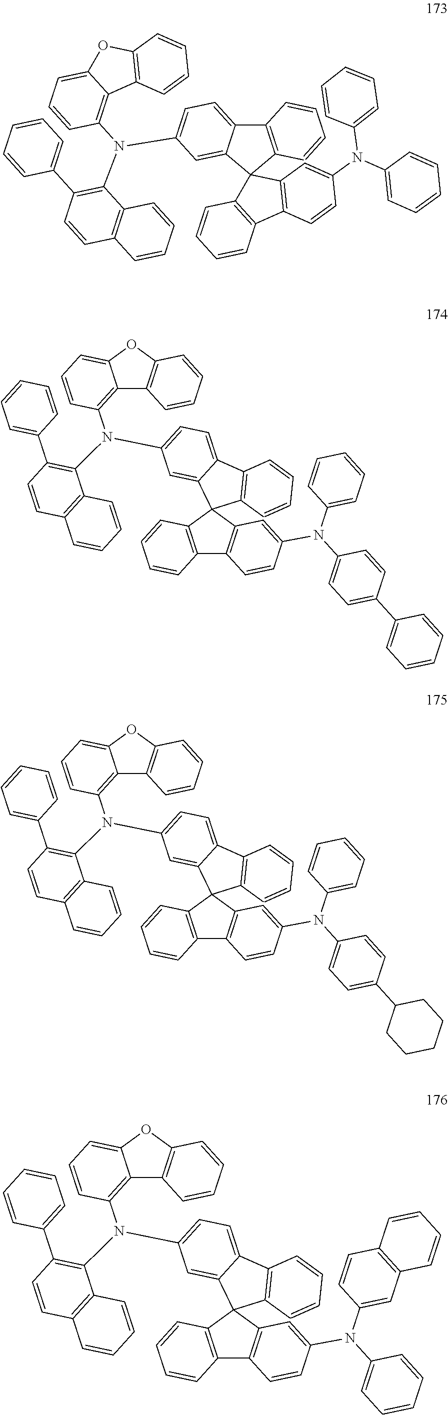 [SMILES: c1ccc(-c2ccc3ccccc3c2N(c2ccc3c(c2)C2(c4ccccc4-c4ccc(N(c5ccccc5)c5ccccc5)cc42)c2ccccc2-3)c2cccc3oc4ccccc4c23)cc1 ; c1ccc(-c2ccc(N(c3ccccc3)c3ccc4c(c3)C3(c5ccccc5-4)c4ccccc4-c4ccc(N(c5c(-c6ccccc6)ccc6ccccc56)c5cccc6oc7ccccc7c56)cc43)cc2)cc1 ; c1ccc(-c2ccc3ccccc3c2N(c2ccc3c(c2)C2(c4ccccc4-c4ccc(N(c5ccccc5)c5ccc(C6CCCCC6)cc5)cc42)c2ccccc2-3)c2cccc3oc4ccccc4c23)cc1 ; c1ccc(-c2ccc3ccccc3c2N(c2ccc3c(c2)C2(c4ccccc4-c4ccc(N(c5ccccc5)c5ccc6ccccc6c5)cc42)c2ccccc2-3)c2cccc3oc4ccccc4c23)cc1]

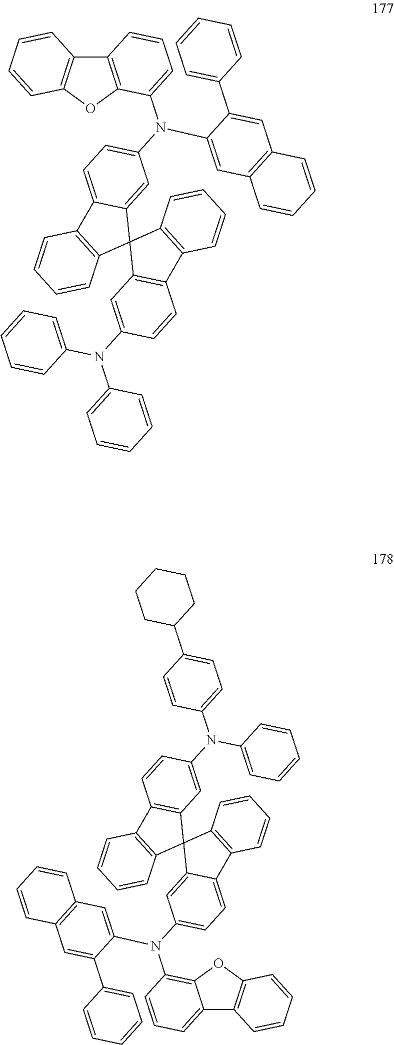 [SMILES: c1ccc(-c2cc3ccccc3cc2N(c2ccc3c(c2)C2(c4ccccc4-c4ccc(N(c5ccccc5)c5ccccc5)cc42)c2ccccc2-3)c2cccc3c2oc2ccccc23)cc1 ; c1ccc(-c2cc3ccccc3cc2N(c2ccc3c(c2)C2(c4ccccc4-c4ccc(N(c5ccccc5)c5ccc(C6CCCCC6)cc5)cc42)c2ccccc2-3)c2cccc3c2oc2ccccc23)cc1]

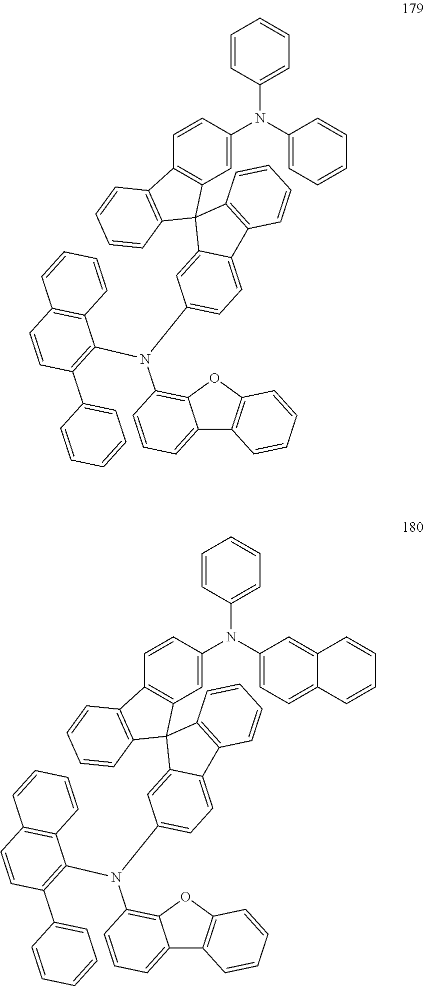 [SMILES: c1ccc(-c2ccc3ccccc3c2N(c2ccc3c(c2)C2(c4ccccc4-c4ccc(N(c5ccccc5)c5ccccc5)cc42)c2ccccc2-3)c2cccc3c2oc2ccccc23)cc1 ; c1ccc(-c2ccc3ccccc3c2N(c2ccc3c(c2)C2(c4ccccc4-c4ccc(N(c5ccccc5)c5ccc6ccccc6c5)cc42)c2ccccc2-3)c2cccc3c2oc2ccccc23)cc1]

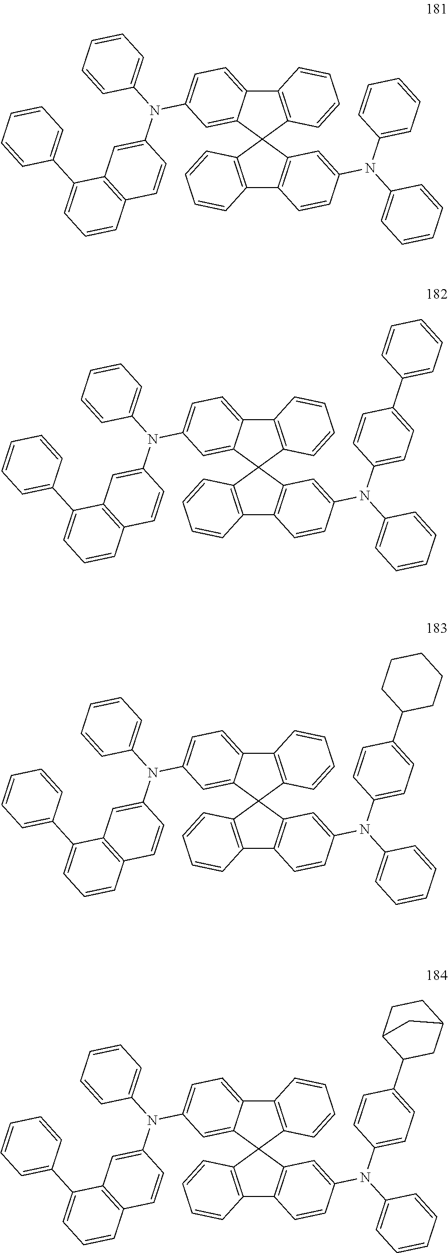 [SMILES: c1ccc(-c2cccc3ccc(N(c4ccccc4)c4ccc5c(c4)C4(c6ccccc6-c6ccc(N(c7ccccc7)c7ccccc7)cc64)c4ccccc4-5)cc23)cc1 ; c1ccc(-c2ccc(N(c3ccccc3)c3ccc4c(c3)C3(c5ccccc5-4)c4ccccc4-c4ccc(N(c5ccccc5)c5ccc6cccc(-c7ccccc7)c6c5)cc43)cc2)cc1 ; c1ccc(-c2cccc3ccc(N(c4ccccc4)c4ccc5c(c4)C4(c6ccccc6-c6ccc(N(c7ccccc7)c7ccc(C8CCCCC8)cc7)cc64)c4ccccc4-5)cc23)cc1 ; c1ccc(-c2cccc3ccc(N(c4ccccc4)c4ccc5c(c4)C4(c6ccccc6-c6ccc(N(c7ccccc7)c7ccc(C8CC9CCC8C9)cc7)cc64)c4ccccc4-5)cc23)cc1]

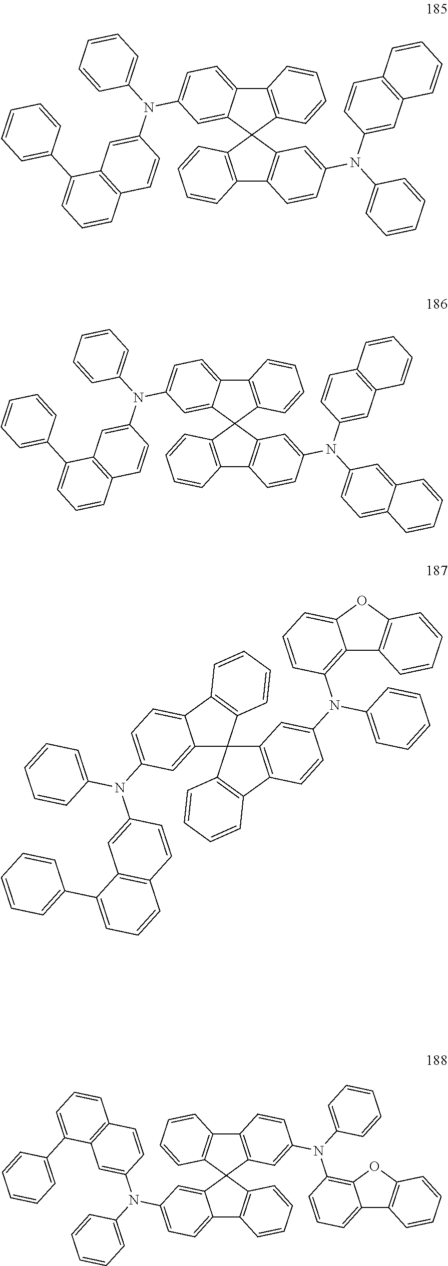 [SMILES: c1ccc(-c2cccc3ccc(N(c4ccccc4)c4ccc5c(c4)C4(c6ccccc6-c6ccc(N(c7ccccc7)c7ccc8ccccc8c7)cc64)c4ccccc4-5)cc23)cc1 ; c1ccc(-c2cccc3ccc(N(c4ccccc4)c4ccc5c(c4)C4(c6ccccc6-c6ccc(N(c7ccc8ccccc8c7)c7ccc8ccccc8c7)cc64)c4ccccc4-5)cc23)cc1 ; c1ccc(-c2cccc3ccc(N(c4ccccc4)c4ccc5c(c4)C4(c6ccccc6-5)c5ccccc5-c5ccc(N(c6ccccc6)c6cccc7oc8ccccc8c67)cc54)cc23)cc1 ; c1ccc(-c2cccc3ccc(N(c4ccccc4)c4ccc5c(c4)C4(c6ccccc6-5)c5ccccc5-c5ccc(N(c6ccccc6)c6cccc7c6oc6ccccc67)cc54)cc23)cc1]

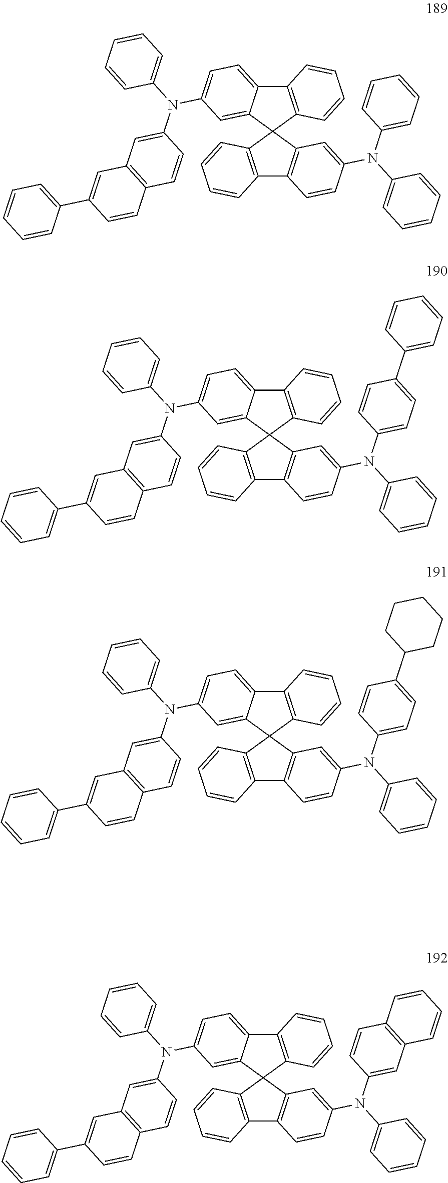 [SMILES: c1ccc(-c2ccc3ccc(N(c4ccccc4)c4ccc5c(c4)C4(c6ccccc6-c6ccc(N(c7ccccc7)c7ccccc7)cc64)c4ccccc4-5)cc3c2)cc1 ; c1ccc(-c2ccc(N(c3ccccc3)c3ccc4c(c3)C3(c5ccccc5-4)c4ccccc4-c4ccc(N(c5ccccc5)c5ccc6ccc(-c7ccccc7)cc6c5)cc43)cc2)cc1 ; c1ccc(-c2ccc3ccc(N(c4ccccc4)c4ccc5c(c4)C4(c6ccccc6-c6ccc(N(c7ccccc7)c7ccc(C8CCCCC8)cc7)cc64)c4ccccc4-5)cc3c2)cc1 ; c1ccc(-c2ccc3ccc(N(c4ccccc4)c4ccc5c(c4)C4(c6ccccc6-c6ccc(N(c7ccccc7)c7ccc8ccccc8c7)cc64)c4ccccc4-5)cc3c2)cc1]

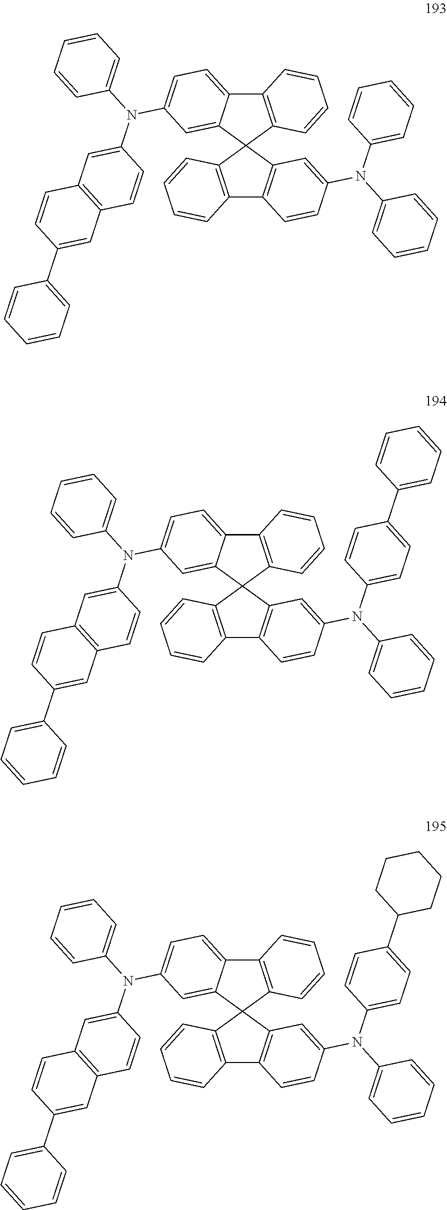 [SMILES: c1ccc(-c2ccc3cc(N(c4ccccc4)c4ccc5c(c4)C4(c6ccccc6-c6ccc(N(c7ccccc7)c7ccccc7)cc64)c4ccccc4-5)ccc3c2)cc1 ; c1ccc(-c2ccc(N(c3ccccc3)c3ccc4c(c3)C3(c5ccccc5-4)c4ccccc4-c4ccc(N(c5ccccc5)c5ccc6cc(-c7ccccc7)ccc6c5)cc43)cc2)cc1 ; c1ccc(-c2ccc3cc(N(c4ccccc4)c4ccc5c(c4)C4(c6ccccc6-c6ccc(N(c7ccccc7)c7ccc(C8CCCCC8)cc7)cc64)c4ccccc4-5)ccc3c2)cc1]

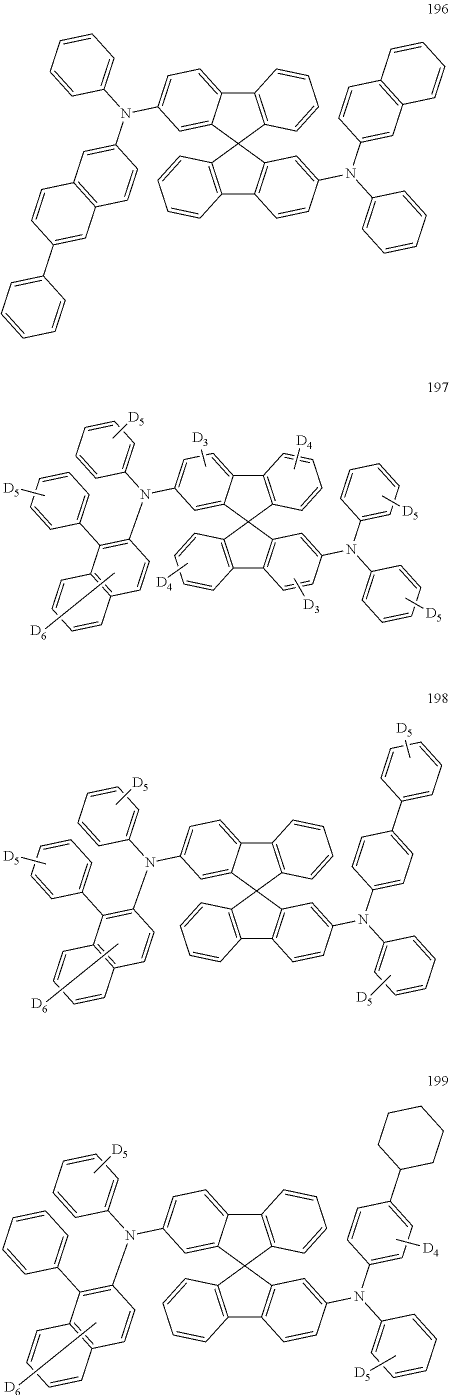

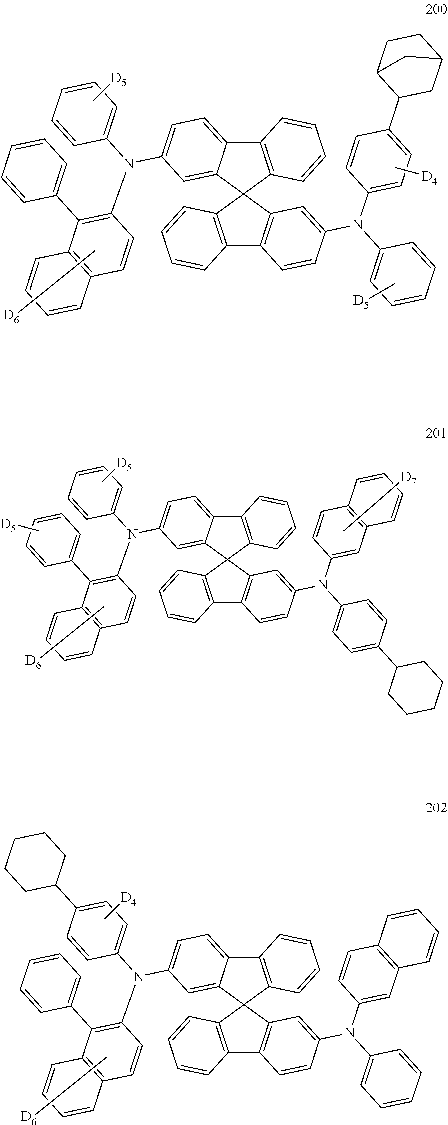

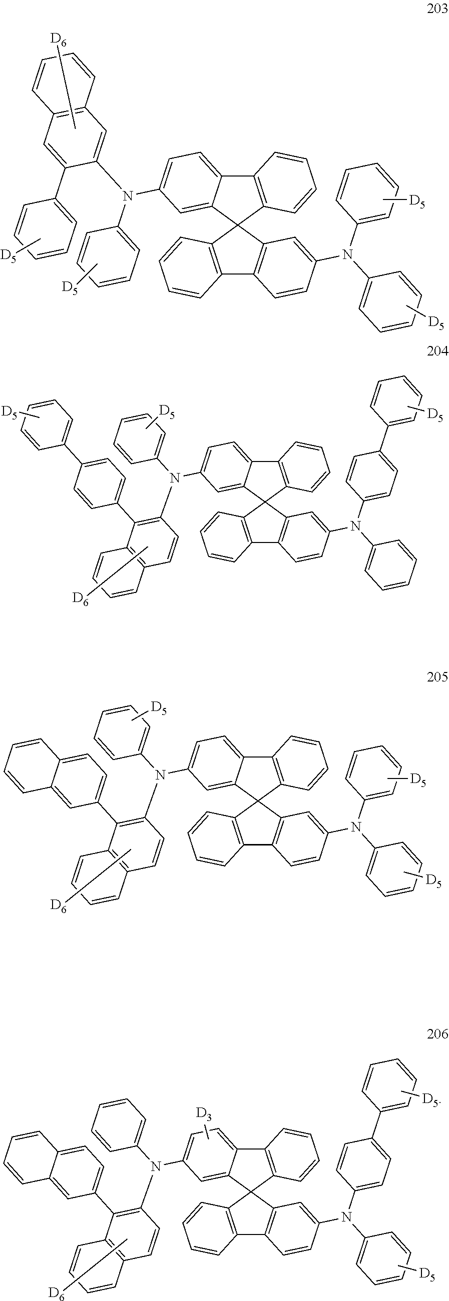

In an embodiment, the second compound may be one of Compounds ETH1 to ETH100:

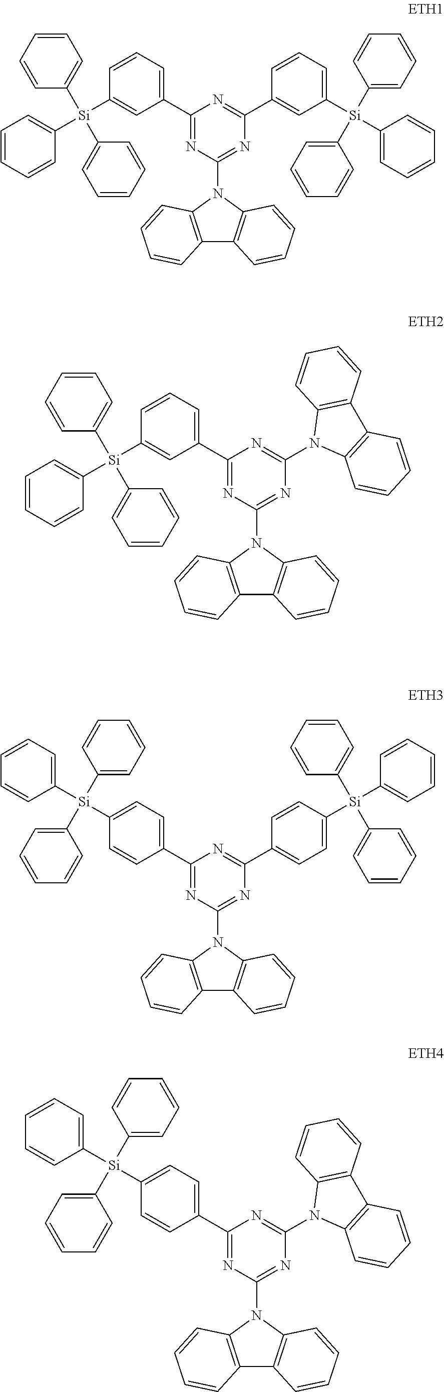 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5ccccc54)n3)c2)cc1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5ccccc54)n3)cc2)cc1 ; c1ccc([Si](c2ccccc2)(c2ccccc2)c2ccc(-c3nc(-n4c5ccccc5c5ccccc54)nc(-n4c5ccccc5c5ccccc54)n3)cc2)cc1]

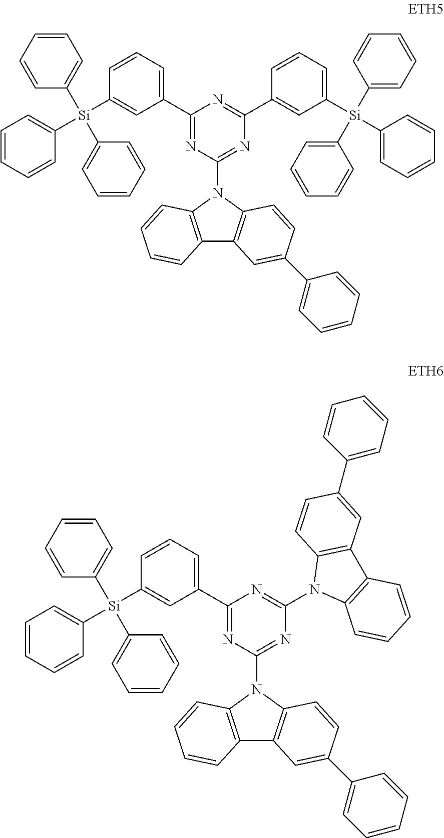 [SMILES: c1ccc(-c2ccc3c(c2)c2ccccc2n3-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; c1ccc(-c2ccc3c(c2)c2ccccc2n3-c2nc(-c3cccc([Si](c4ccccc4)(c4ccccc4)c4ccccc4)c3)nc(-n3c4ccccc4c4cc(-c5ccccc5)ccc43)n2)cc1]

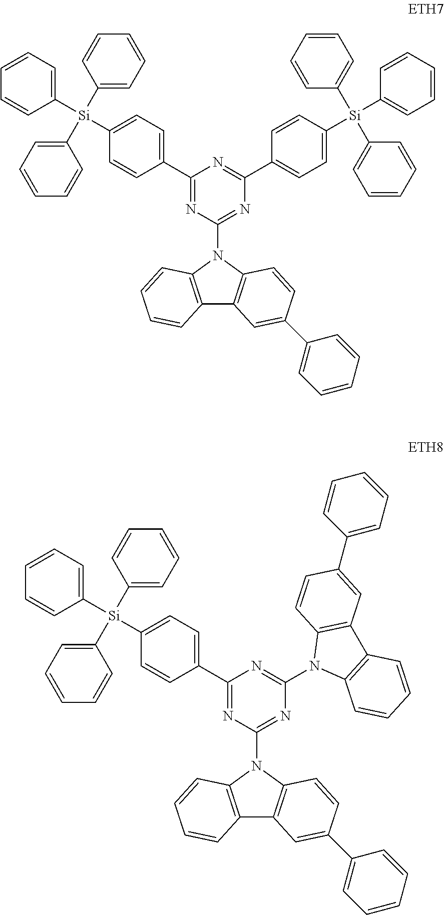 [SMILES: c1ccc(-c2ccc3c(c2)c2ccccc2n3-c2nc(-c3ccc([Si](c4ccccc4)(c4ccccc4)c4ccccc4)cc3)nc(-c3ccc([Si](c4ccccc4)(c4ccccc4)c4ccccc4)cc3)n2)cc1 ; c1ccc(-c2ccc3c(c2)c2ccccc2n3-c2nc(-c3ccc([Si](c4ccccc4)(c4ccccc4)c4ccccc4)cc3)nc(-n3c4ccccc4c4cc(-c5ccccc5)ccc43)n2)cc1]

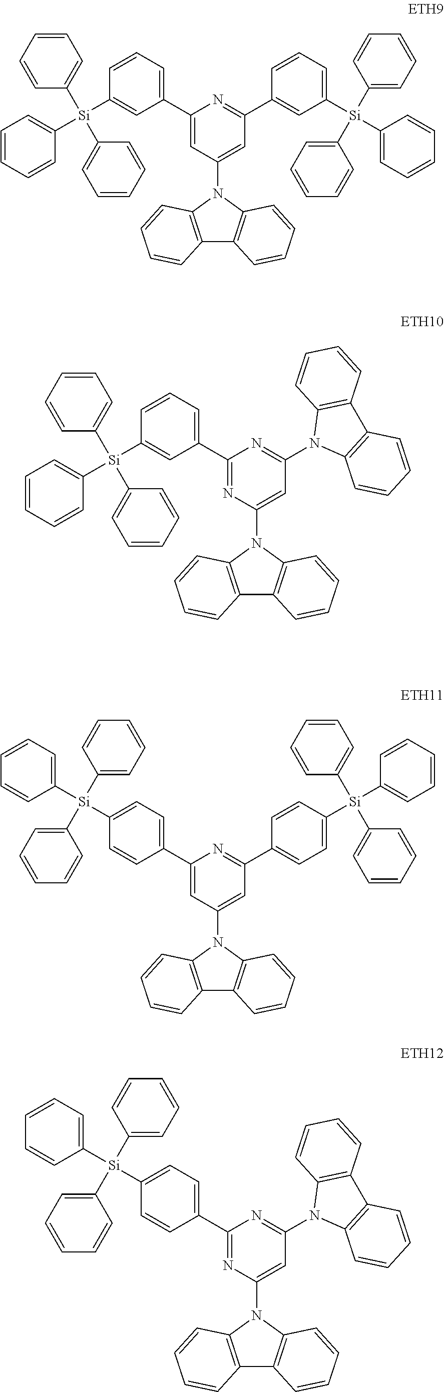 [SMILES: c1ccc([Si](c2ccccc2)(c2ccccc2)c2cccc(-c3cc(-n4c5ccccc5c5ccccc54)cc(-c4cccc([Si](c5ccccc5)(c5ccccc5)c5ccccc5)c4)n3)c2)cc1 ; c1ccc([Si](c2ccccc2)(c2ccccc2)c2cccc(-c3nc(-n4c5ccccc5c5ccccc54)cc(-n4c5ccccc5c5ccccc54)n3)c2)cc1 ; c1ccc([Si](c2ccccc2)(c2ccccc2)c2ccc(-c3cc(-n4c5ccccc5c5ccccc54)cc(-c4ccc([Si](c5ccccc5)(c5ccccc5)c5ccccc5)cc4)n3)cc2)cc1 ; c1ccc([Si](c2ccccc2)(c2ccccc2)c2ccc(-c3nc(-n4c5ccccc5c5ccccc54)cc(-n4c5ccccc5c5ccccc54)n3)cc2)cc1]

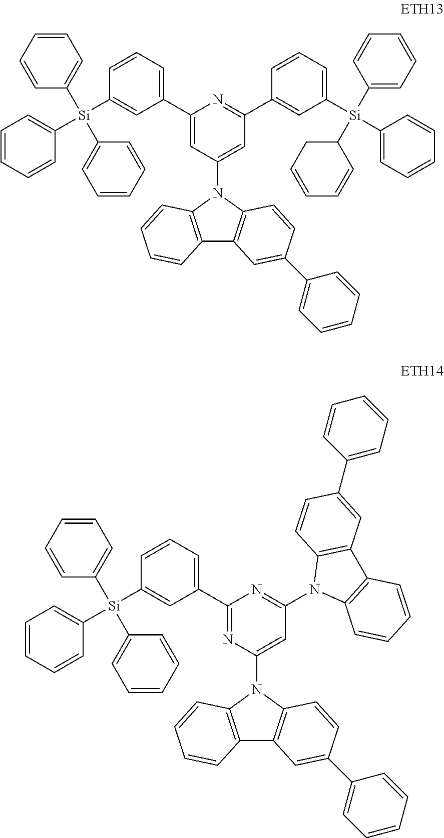 [SMILES: C1=CCC([Si](c2ccccc2)(c2ccccc2)c2cccc(-c3cc(-n4c5ccccc5c5cc(-c6ccccc6)ccc54)cc(-c4cccc([Si](c5ccccc5)(c5ccccc5)c5ccccc5)c4)n3)c2)C=C1 ; c1ccc(-c2ccc3c(c2)c2ccccc2n3-c2cc(-n3c4ccccc4c4cc(-c5ccccc5)ccc43)nc(-c3cccc([Si](c4ccccc4)(c4ccccc4)c4ccccc4)c3)n2)cc1]

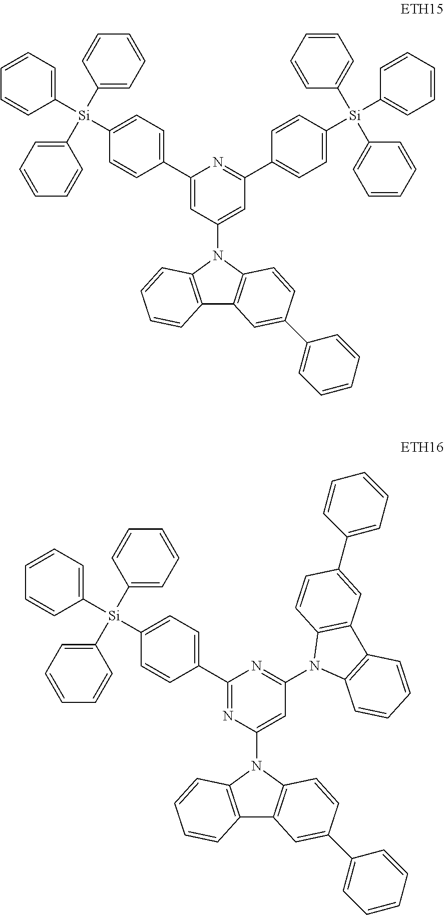 [SMILES: c1ccc(-c2ccc3c(c2)c2ccccc2n3-c2cc(-c3ccc([Si](c4ccccc4)(c4ccccc4)c4ccccc4)cc3)nc(-c3ccc([Si](c4ccccc4)(c4ccccc4)c4ccccc4)cc3)c2)cc1 ; c1ccc(-c2ccc3c(c2)c2ccccc2n3-c2cc(-n3c4ccccc4c4cc(-c5ccccc5)ccc43)nc(-c3ccc([Si](c4ccccc4)(c4ccccc4)c4ccccc4)cc3)n2)cc1]

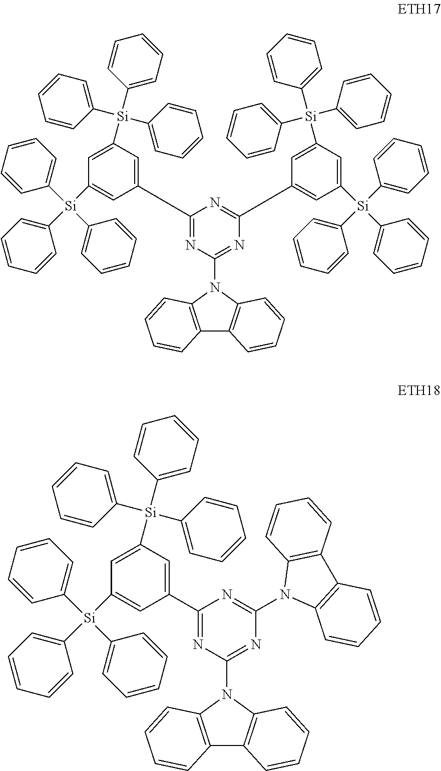 [SMILES: c1ccc([Si](c2ccccc2)(c2ccccc2)c2cc(-c3nc(-c4cc([Si](c5ccccc5)(c5ccccc5)c5ccccc5)cc([Si](c5ccccc5)(c5ccccc5)c5ccccc5)c4)nc(-n4c5ccccc5c5ccccc54)n3)cc([Si](c3ccccc3)(c3ccccc3)c3ccccc3)c2)cc1 ; c1ccc([Si](c2ccccc2)(c2ccccc2)c2cc(-c3nc(-n4c5ccccc5c5ccccc54)nc(-n4c5ccccc5c5ccccc54)n3)cc([Si](c3ccccc3)(c3ccccc3)c3ccccc3)c2)cc1]

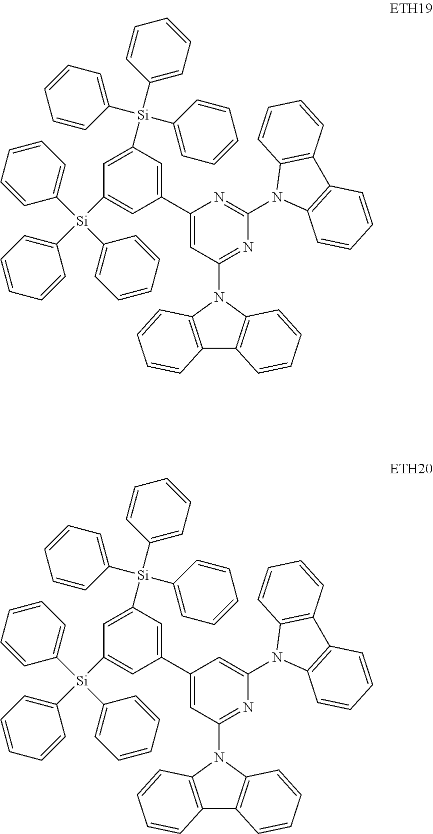 [SMILES: c1ccc([Si](c2ccccc2)(c2ccccc2)c2cc(-c3cc(-n4c5ccccc5c5ccccc54)nc(-n4c5ccccc5c5ccccc54)n3)cc([Si](c3ccccc3)(c3ccccc3)c3ccccc3)c2)cc1 ; c1ccc([Si](c2ccccc2)(c2ccccc2)c2cc(-c3cc(-n4c5ccccc5c5ccccc54)nc(-n4c5ccccc5c5ccccc54)c3)cc([Si](c3ccccc3)(c3ccccc3)c3ccccc3)c2)cc1]

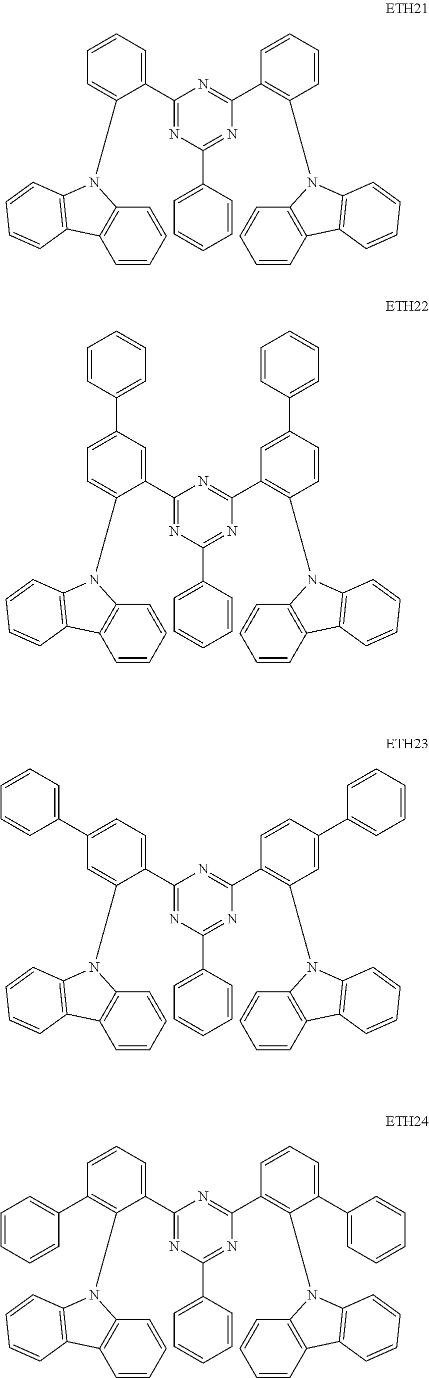 [SMILES: c1ccc(-c2nc(-c3ccccc3-n3c4ccccc4c4ccccc43)nc(-c3ccccc3-n3c4ccccc4c4ccccc43)n2)cc1 ; c1ccc(-c2ccc(-n3c4ccccc4c4ccccc43)c(-c3nc(-c4ccccc4)nc(-c4cc(-c5ccccc5)ccc4-n4c5ccccc5c5ccccc54)n3)c2)cc1 ; c1ccc(-c2ccc(-c3nc(-c4ccccc4)nc(-c4ccc(-c5ccccc5)cc4-n4c5ccccc5c5ccccc54)n3)c(-n3c4ccccc4c4ccccc43)c2)cc1 ; c1ccc(-c2nc(-c3cccc(-c4ccccc4)c3-n3c4ccccc4c4ccccc43)nc(-c3cccc(-c4ccccc4)c3-n3c4ccccc4c4ccccc43)n2)cc1]

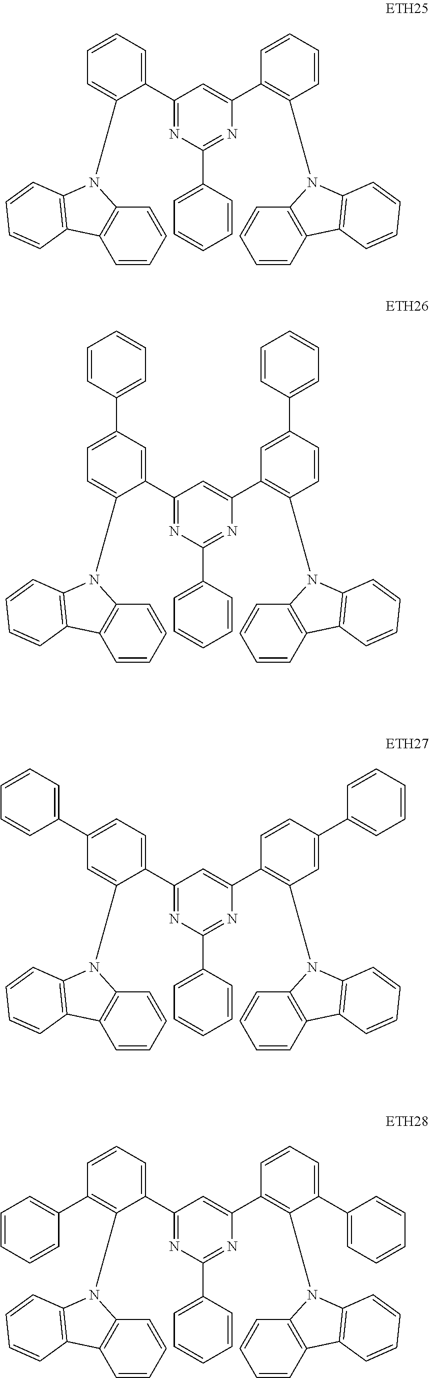 [SMILES: c1ccc(-c2nc(-c3ccccc3-n3c4ccccc4c4ccccc43)cc(-c3ccccc3-n3c4ccccc4c4ccccc43)n2)cc1 ; c1ccc(-c2ccc(-n3c4ccccc4c4ccccc43)c(-c3cc(-c4cc(-c5ccccc5)ccc4-n4c5ccccc5c5ccccc54)nc(-c4ccccc4)n3)c2)cc1 ; c1ccc(-c2ccc(-c3cc(-c4ccc(-c5ccccc5)cc4-n4c5ccccc5c5ccccc54)nc(-c4ccccc4)n3)c(-n3c4ccccc4c4ccccc43)c2)cc1 ; c1ccc(-c2nc(-c3cccc(-c4ccccc4)c3-n3c4ccccc4c4ccccc43)cc(-c3cccc(-c4ccccc4)c3-n3c4ccccc4c4ccccc43)n2)cc1]

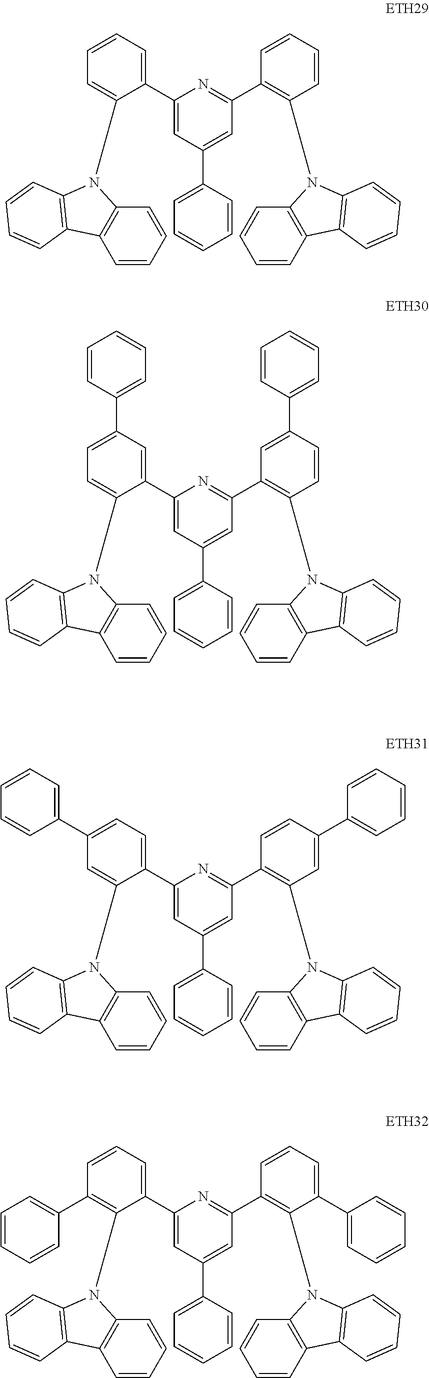 [SMILES: c1ccc(-c2cc(-c3ccccc3-n3c4ccccc4c4ccccc43)nc(-c3ccccc3-n3c4ccccc4c4ccccc43)c2)cc1 ; c1ccc(-c2cc(-c3cc(-c4ccccc4)ccc3-n3c4ccccc4c4ccccc43)nc(-c3cc(-c4ccccc4)ccc3-n3c4ccccc4c4ccccc43)c2)cc1 ; c1ccc(-c2cc(-c3ccc(-c4ccccc4)cc3-n3c4ccccc4c4ccccc43)nc(-c3ccc(-c4ccccc4)cc3-n3c4ccccc4c4ccccc43)c2)cc1 ; c1ccc(-c2cc(-c3cccc(-c4ccccc4)c3-n3c4ccccc4c4ccccc43)nc(-c3cccc(-c4ccccc4)c3-n3c4ccccc4c4ccccc43)c2)cc1]

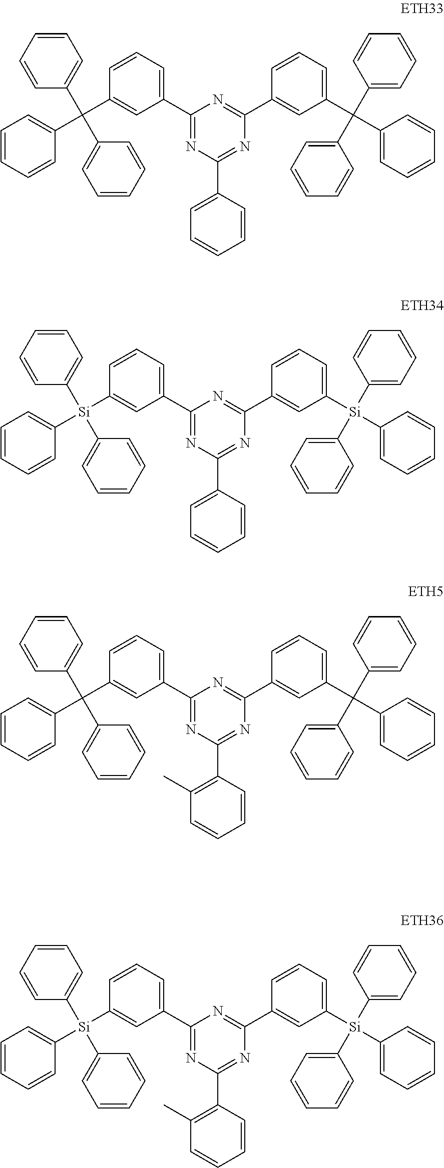 [SMILES: c1ccc(-c2nc(-c3cccc(C(c4ccccc4)(c4ccccc4)c4ccccc4)c3)nc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)nc(-c2cccc([Si](c3ccccc3)(c3ccccc3)c3ccccc3)c2)n1]

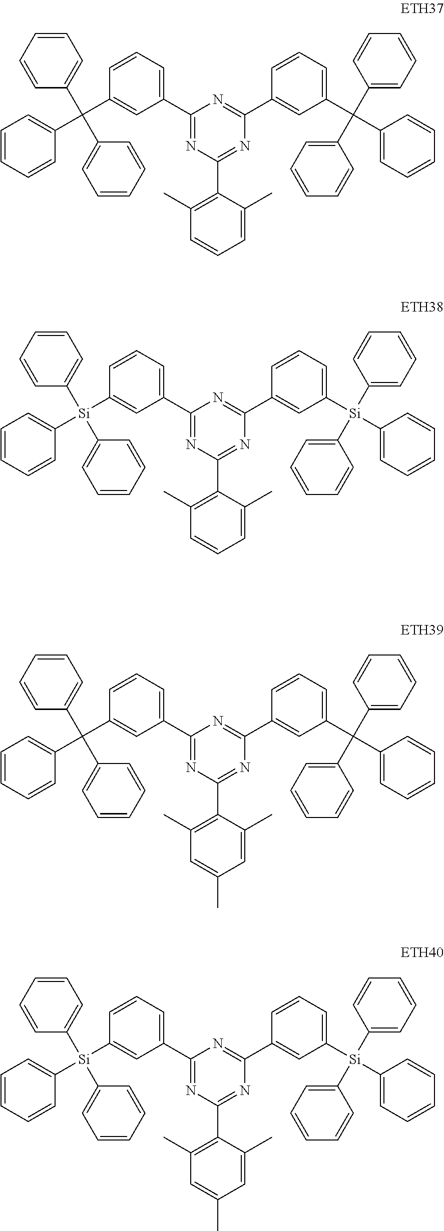 [SMILES: Cc1cccc(C)c1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)nc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)nc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)nc(-c3cccc([Si](c4ccccc4)(c4ccccc4)c4ccccc4)c3)n2)c(C)c1]

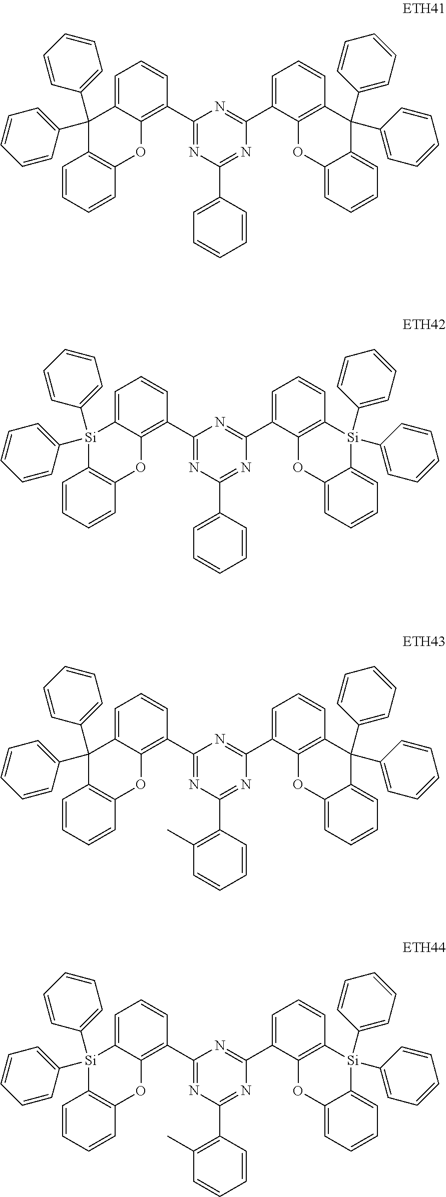 [SMILES: c1ccc(-c2nc(-c3cccc4c3Oc3ccccc3C4(c3ccccc3)c3ccccc3)nc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1]

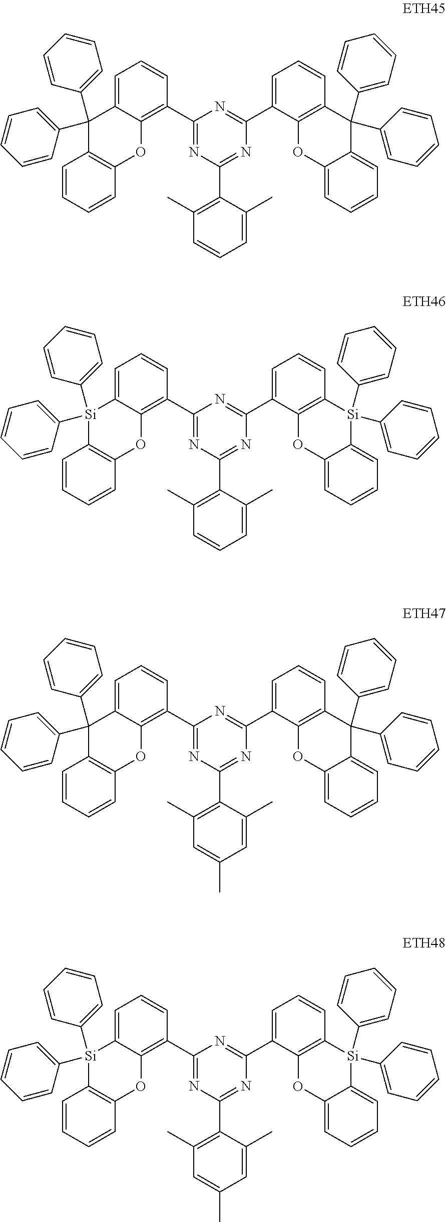 [SMILES: Cc1cccc(C)c1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1 ; Cc1cc(C)c(-c2nc(-c3cccc4c3Oc3ccccc3C4(c3ccccc3)c3ccccc3)nc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)c(C)c1]

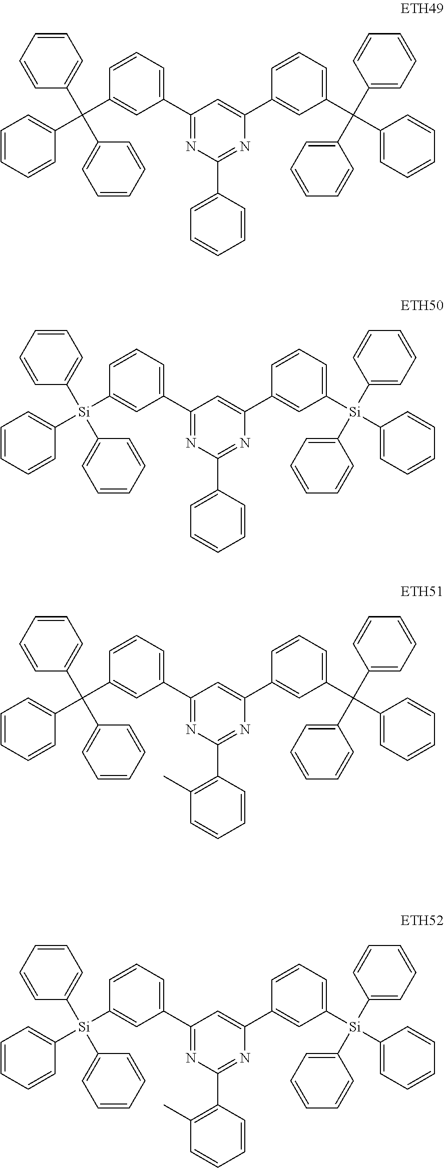 [SMILES: c1ccc(-c2nc(-c3cccc(C(c4ccccc4)(c4ccccc4)c4ccccc4)c3)cc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)cc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)cc(-c2cccc([Si](c3ccccc3)(c3ccccc3)c3ccccc3)c2)n1]

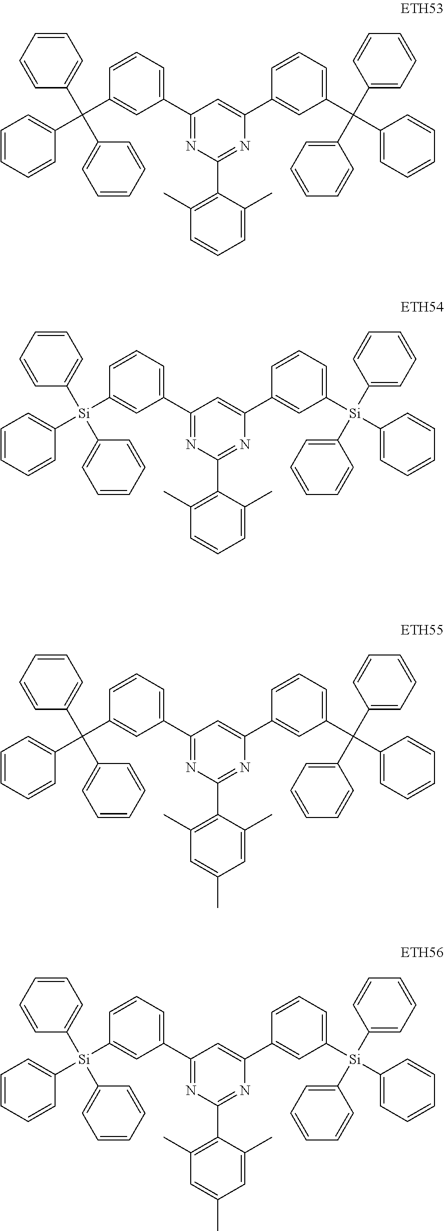 [SMILES: Cc1cccc(C)c1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)cc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)cc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)cc(-c3cccc([Si](c4ccccc4)(c4ccccc4)c4ccccc4)c3)n2)c(C)c1]

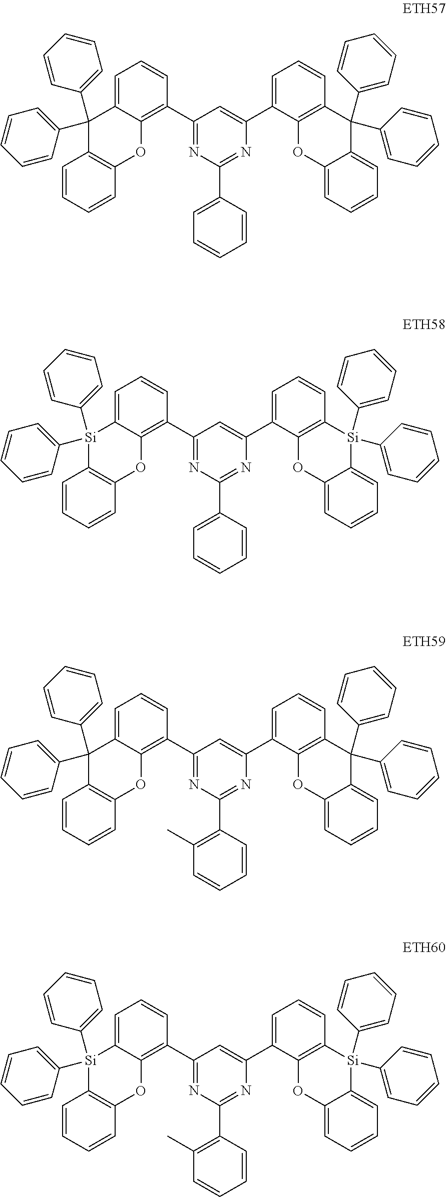 [SMILES: c1ccc(-c2nc(-c3cccc4c3Oc3ccccc3C4(c3ccccc3)c3ccccc3)cc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)cc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)cc(-c2cccc3c2Oc2ccccc2[Si]3(c2ccccc2)c2ccccc2)n1]

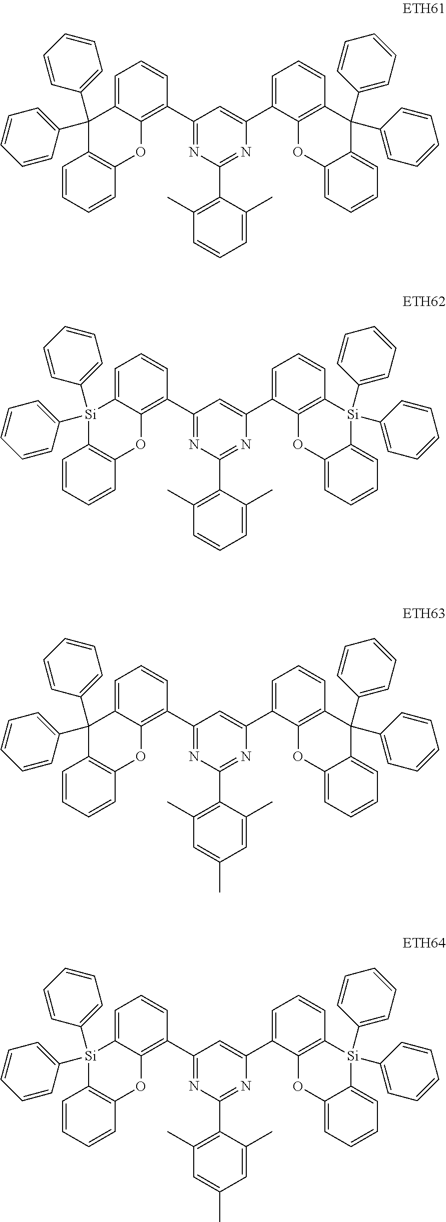 [SMILES: Cc1cccc(C)c1-c1nc(-c2cccc3c2Oc2ccccc2C3(c2ccccc2)c2ccccc2)cc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)cc(-c2cccc3c2Oc2ccccc2[Si]3(c2ccccc2)c2ccccc2)n1 ; Cc1cc(C)c(-c2nc(-c3cccc4c3Oc3ccccc3C4(c3ccccc3)c3ccccc3)cc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)c(C)c1]

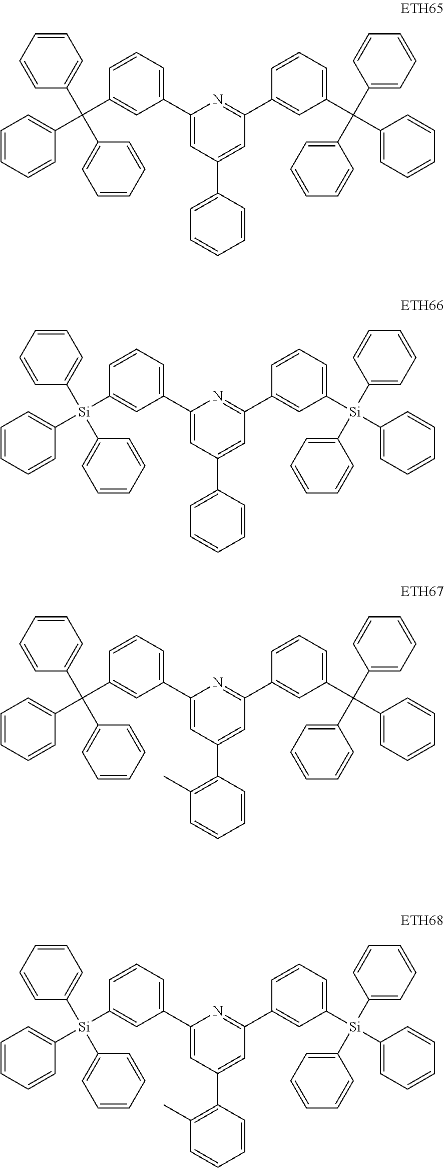 [SMILES: c1ccc(-c2cc(-c3cccc(C(c4ccccc4)(c4ccccc4)c4ccccc4)c3)nc(-c3cccc(C(c4ccccc4)(c4ccccc4)c4ccccc4)c3)c2)cc1 ; c1ccc(-c2cc(-c3cccc([Si](c4ccccc4)(c4ccccc4)c4ccccc4)c3)nc(-c3cccc([Si](c4ccccc4)(c4ccccc4)c4ccccc4)c3)c2)cc1 ; Cc1ccccc1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1ccccc1-c1cc(-c2cccc([Si](c3ccccc3)(c3ccccc3)c3ccccc3)c2)nc(-c2cccc([Si](c3ccccc3)(c3ccccc3)c3ccccc3)c2)c1]

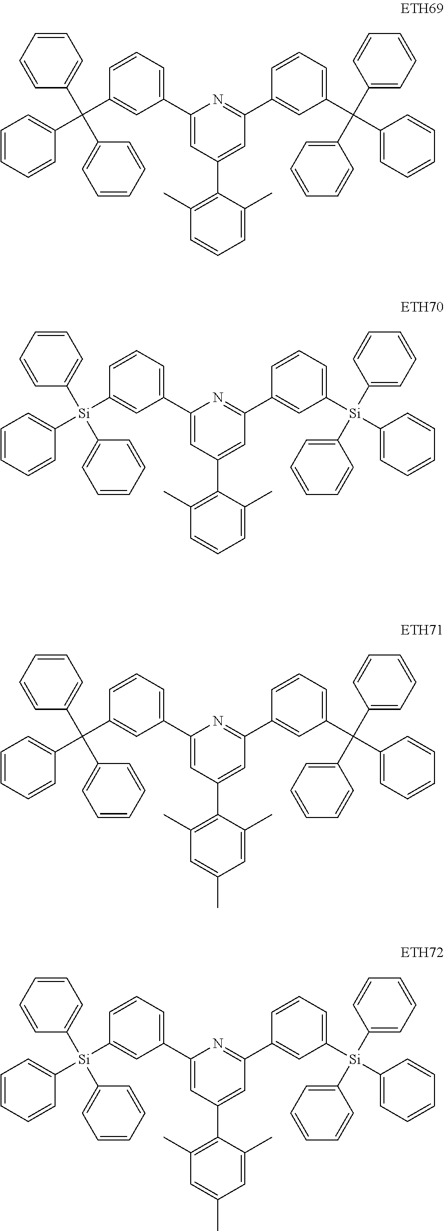 [SMILES: Cc1cccc(C)c1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1cccc(C)c1-c1cc(-c2cccc([Si](c3ccccc3)(c3ccccc3)c3ccccc3)c2)nc(-c2cccc([Si](c3ccccc3)(c3ccccc3)c3ccccc3)c2)c1 ; Cc1cc(C)c(-c2cc(-c3cccc(C(c4ccccc4)(c4ccccc4)c4ccccc4)c3)nc(-c3cccc(C(c4ccccc4)(c4ccccc4)c4ccccc4)c3)c2)c(C)c1 ; Cc1cc(C)c(-c2cc(-c3cccc([Si](c4ccccc4)(c4ccccc4)c4ccccc4)c3)nc(-c3cccc([Si](c4ccccc4)(c4ccccc4)c4ccccc4)c3)c2)c(C)c1]

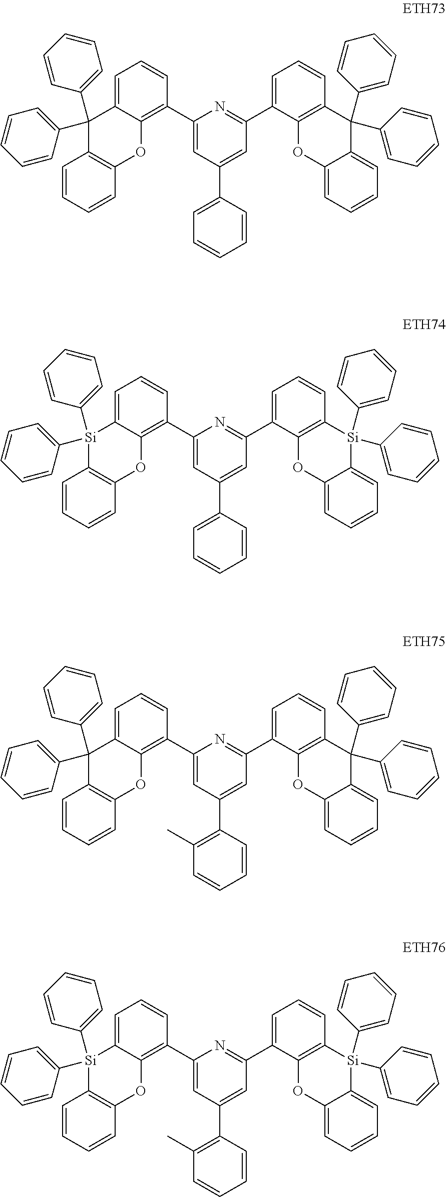 [SMILES: c1ccc(-c2cc(-c3cccc4c3Oc3ccccc3C4(c3ccccc3)c3ccccc3)nc(-c3cccc4c3Oc3ccccc3C4(c3ccccc3)c3ccccc3)c2)cc1 ; c1ccc(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)cc1 ; Cc1ccccc1-c1cc(-c2cccc3c2Oc2ccccc2C3(c2ccccc2)c2ccccc2)nc(-c2cccc3c2Oc2ccccc2C3(c2ccccc2)c2ccccc2)c1 ; Cc1ccccc1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1]

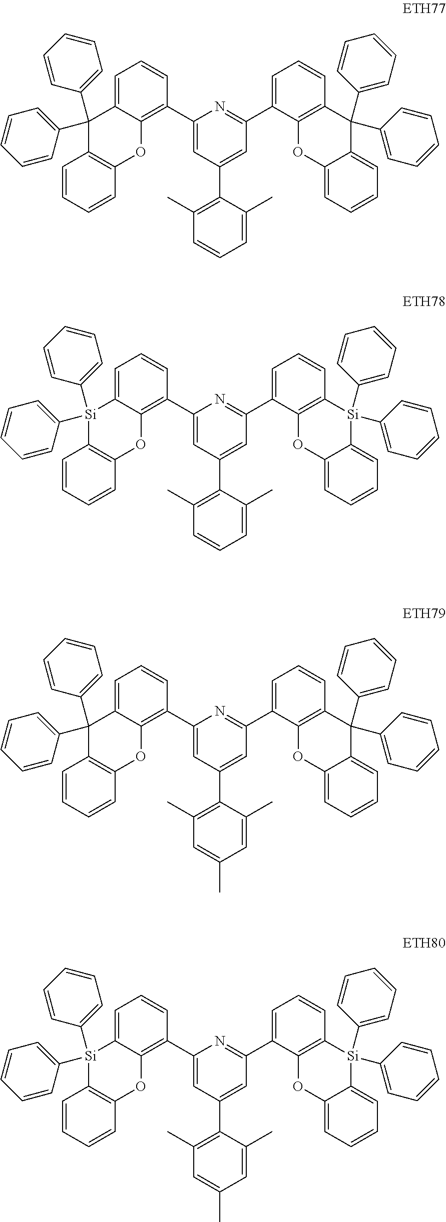 [SMILES: Cc1cccc(C)c1-c1cc(-c2cccc3c2Oc2ccccc2C3(c2ccccc2)c2ccccc2)nc(-c2cccc3c2Oc2ccccc2C3(c2ccccc2)c2ccccc2)c1 ; Cc1cccc(C)c1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1 ; Cc1cc(C)c(-c2cc(-c3cccc4c3Oc3ccccc3C4(c3ccccc3)c3ccccc3)nc(-c3cccc4c3Oc3ccccc3C4(c3ccccc3)c3ccccc3)c2)c(C)c1 ; Cc1cc(C)c(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)c(C)c1]

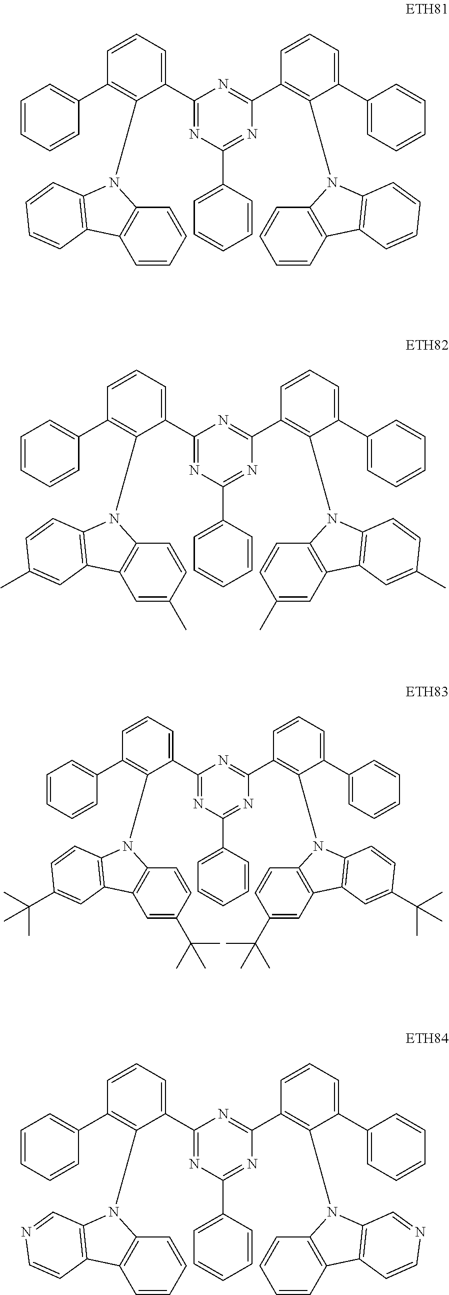

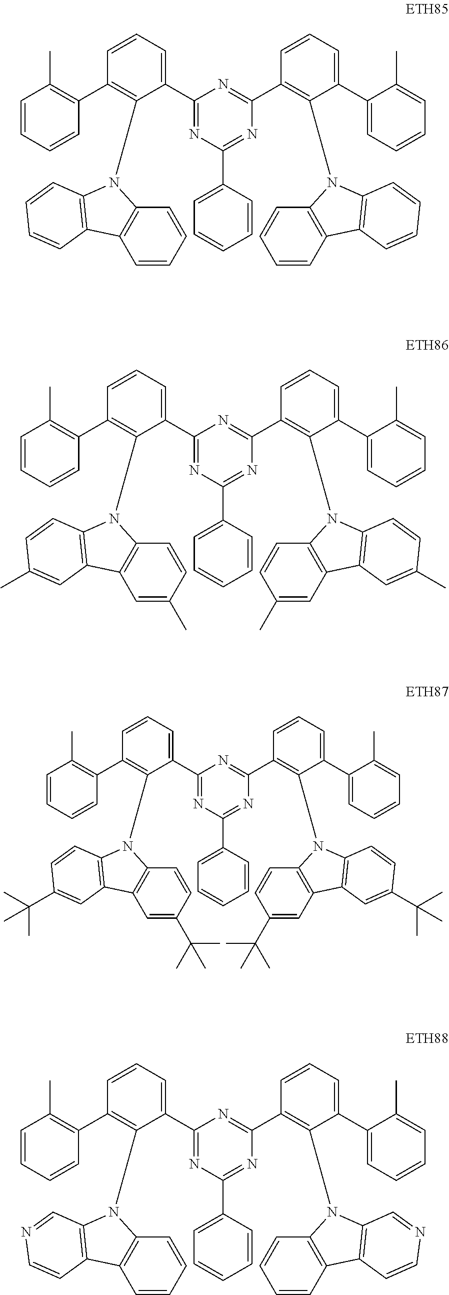

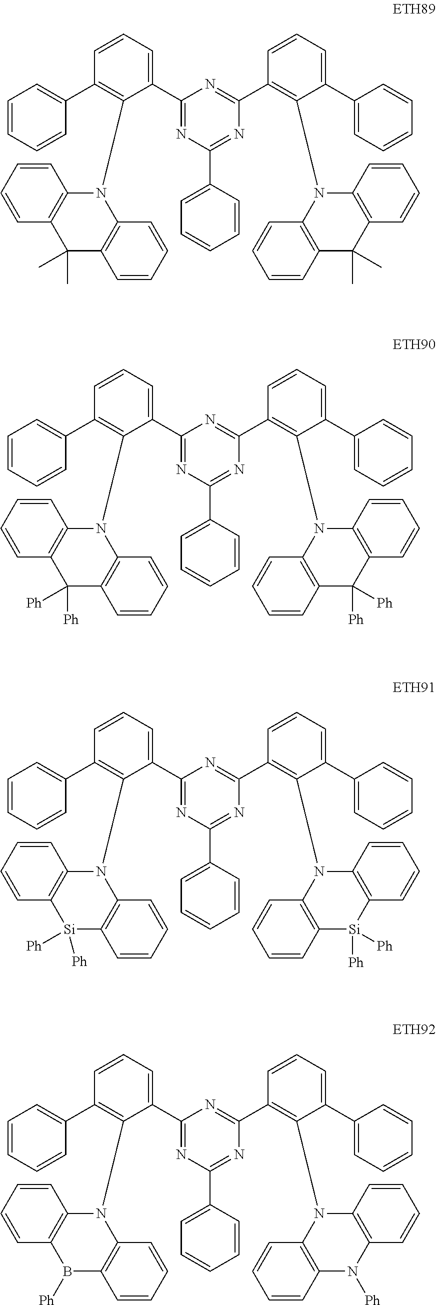

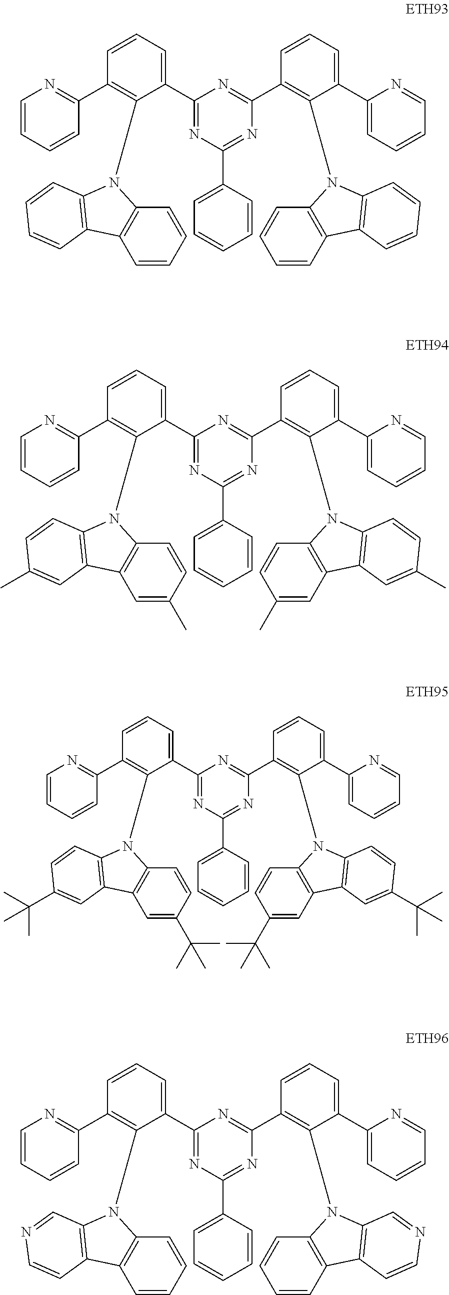

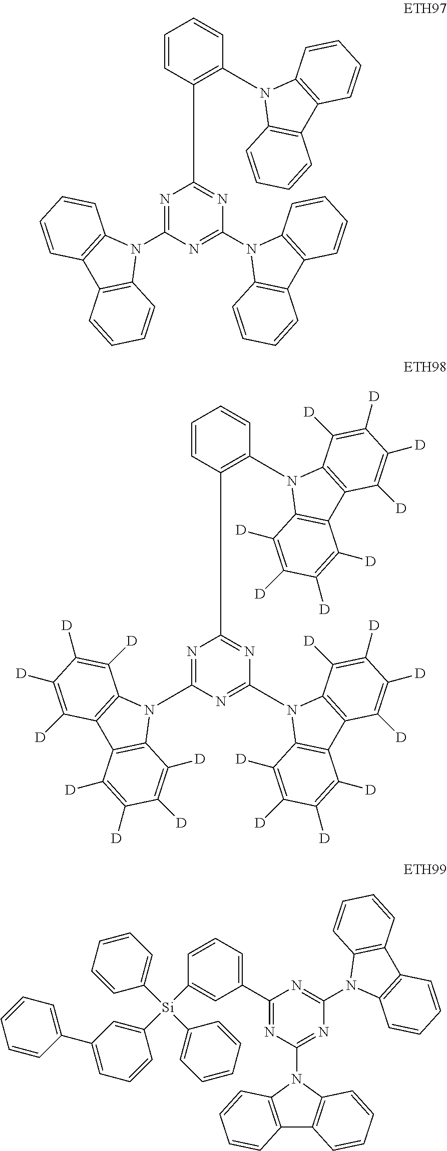 [SMILES: c1ccc(-n2c3ccccc3c3ccccc32)c(-c2nc(-n3c4ccccc4c4ccccc43)nc(-n3c4ccccc4c4ccccc43)n2)c1 ; [2H]c1c([2H])c([2H])c2c(c1[2H])c1c([2H])c([2H])c([2H])c([2H])c1n2-c1nc(-c2ccccc2-n2c3c([2H])c([2H])c([2H])c([2H])c3c3c([2H])c([2H])c([2H])c([2H])c32)nc(-n2c3c([2H])c([2H])c([2H])c([2H])c3c3c([2H])c([2H])c([2H])c([2H])c32)n1 ; c1ccc(-c2cccc([Si](c3ccccc3)(c3ccccc3)c3cccc(-c4nc(-n5c6ccccc6c6ccccc65)nc(-n5c6ccccc6c6ccccc65)n4)c3)c2)cc1]

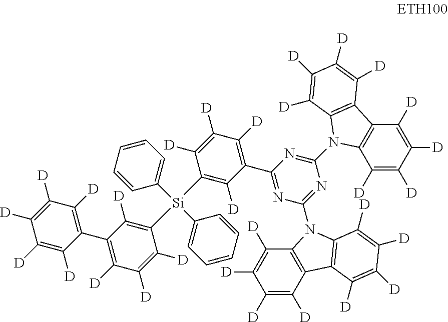 [SMILES: [2H]c1c([2H])c([2H])c(-c2c([2H])c([2H])c([2H])c([Si](c3ccccc3)(c3ccccc3)c3c([2H])c([2H])c([2H])c(-c4nc(-n5c6c([2H])c([2H])c([2H])c([2H])c6c6c([2H])c([2H])c([2H])c([2H])c65)nc(-n5c6c([2H])c([2H])c([2H])c([2H])c6c6c([2H])c([2H])c([2H])c([2H])c65)n4)c3[2H])c2[2H])c([2H])c1[2H]]

In an embodiment, the third compound may be one of Compounds HTH1 to HTH41:

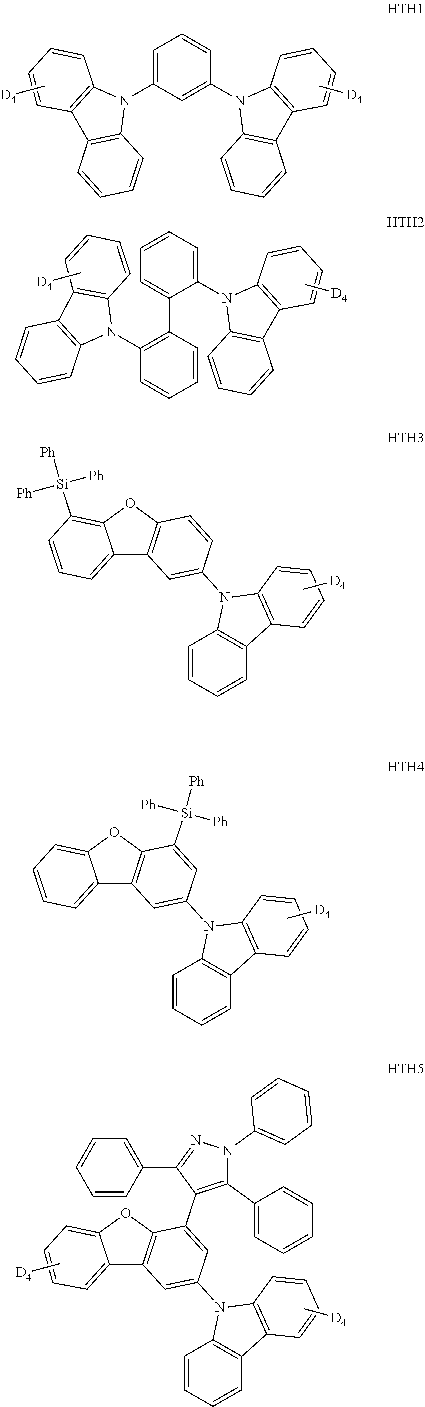

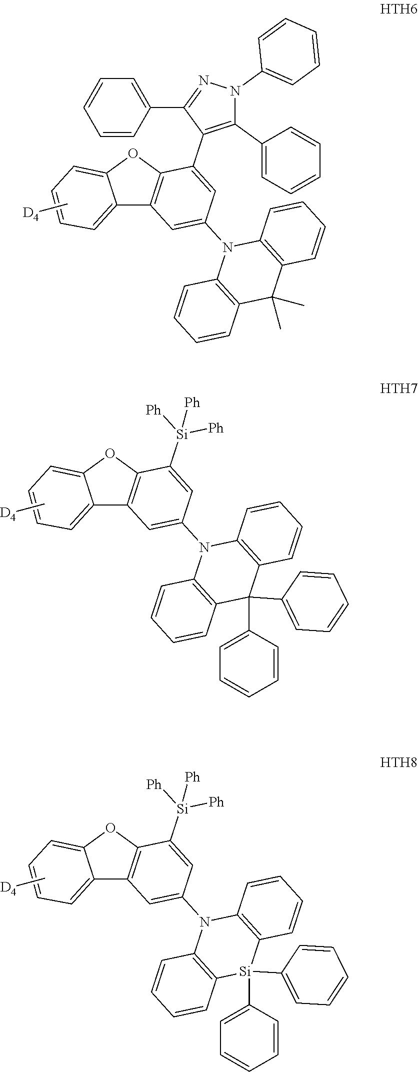

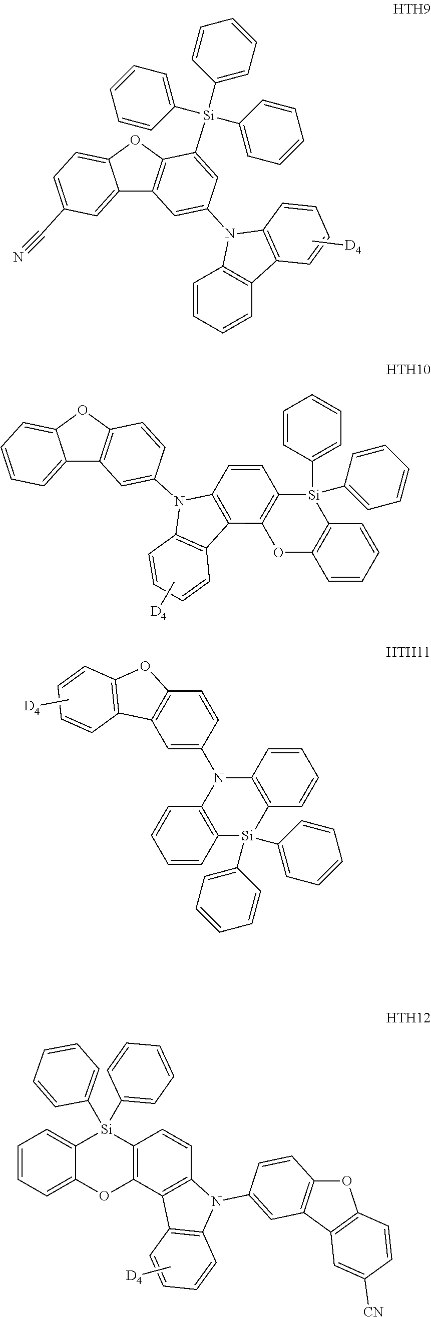

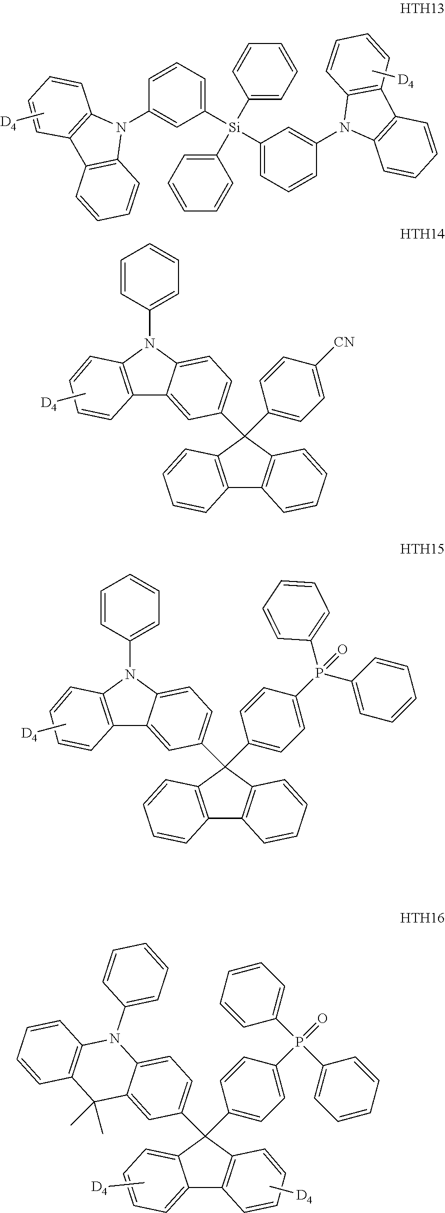

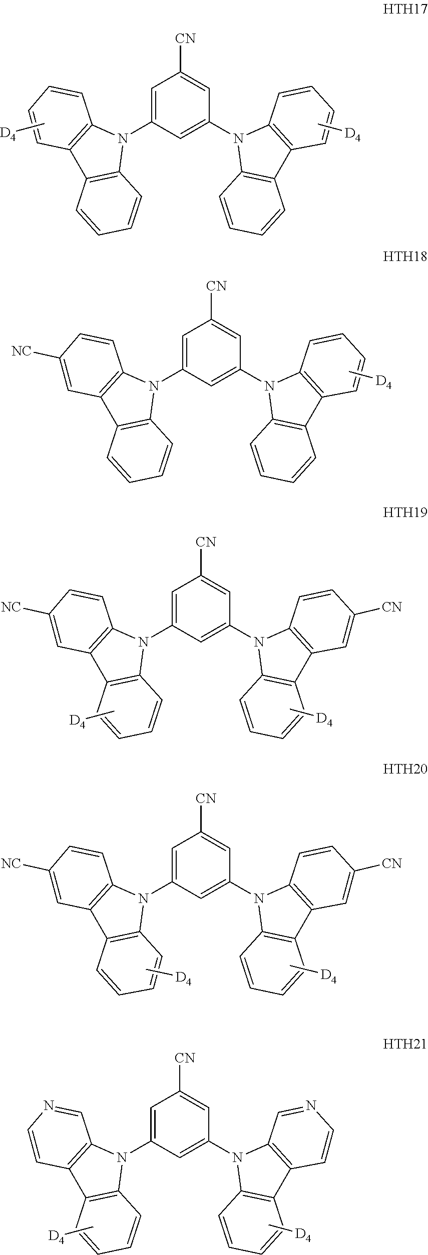

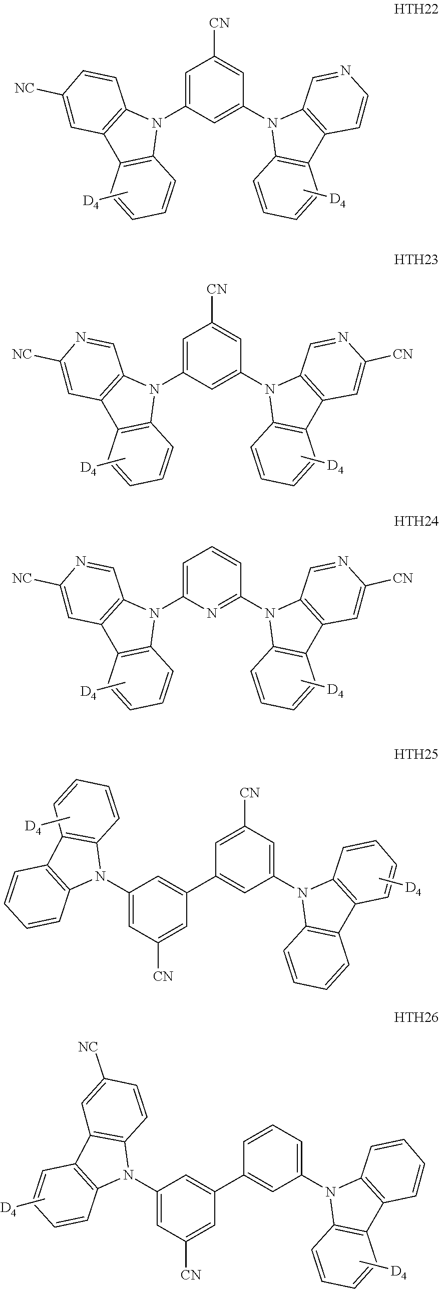

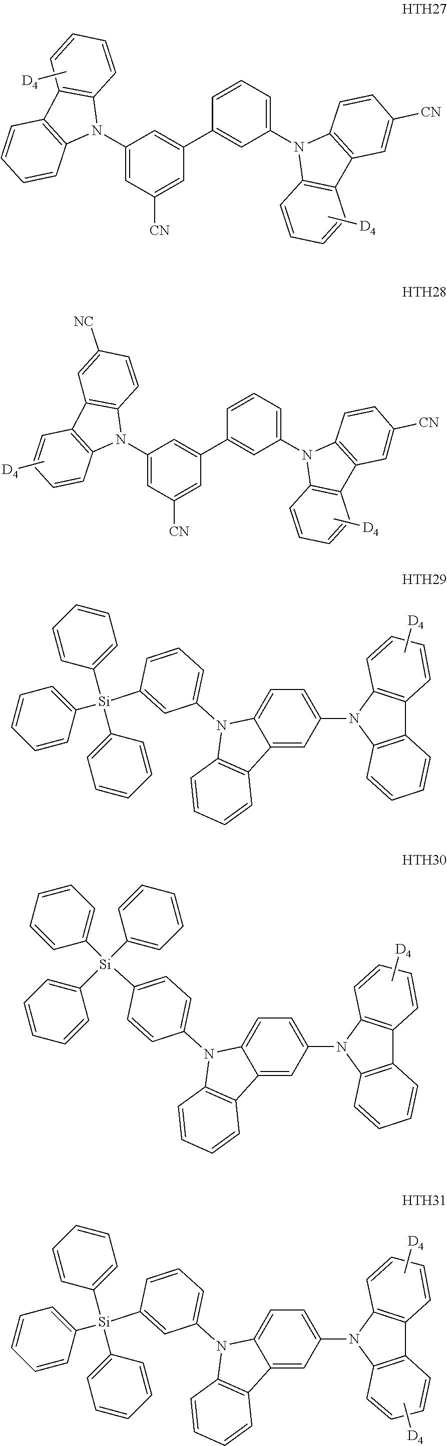

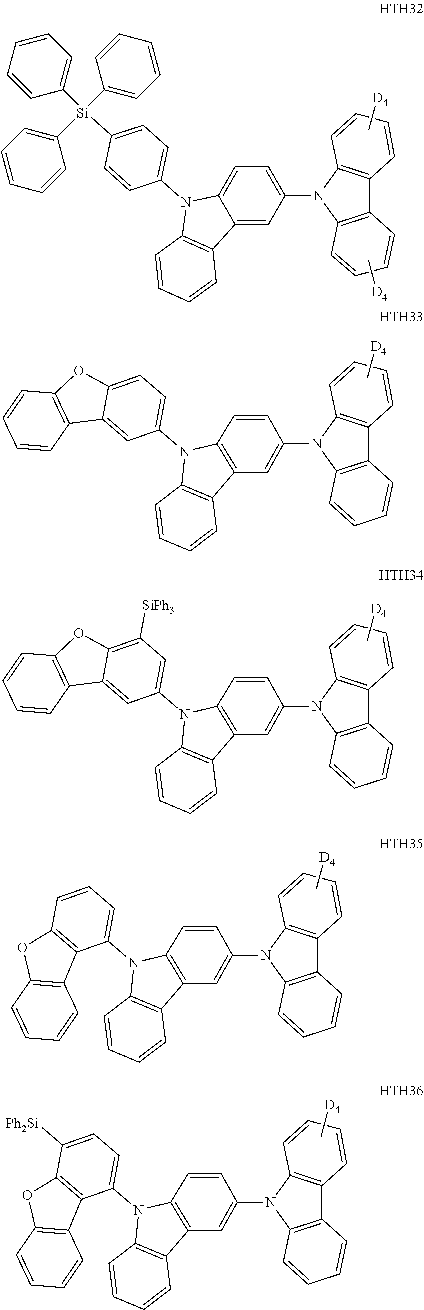

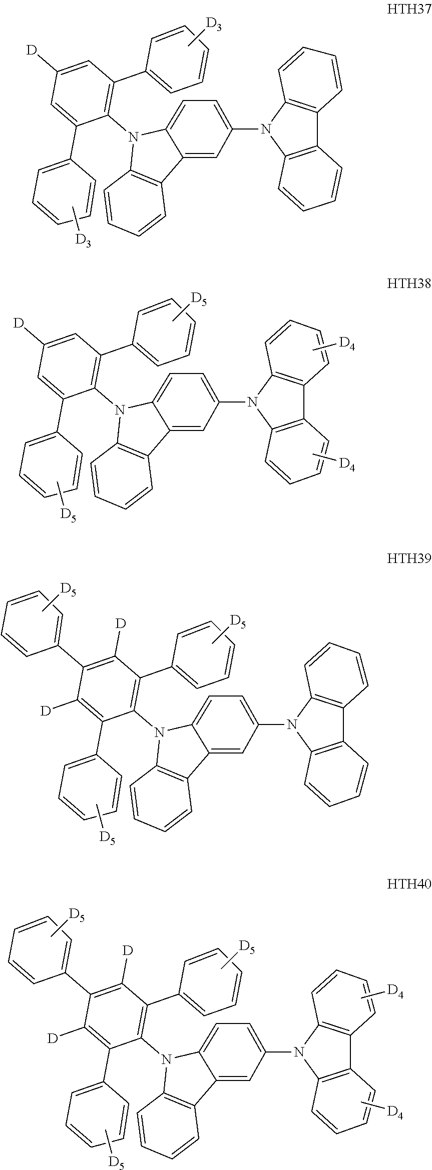

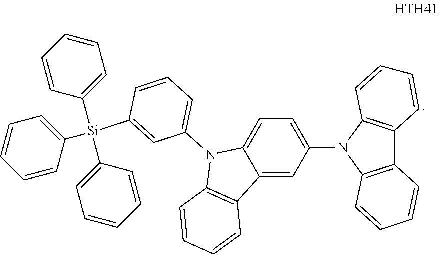 [SMILES: c1ccc([Si](c2ccccc2)(c2ccccc2)c2cccc(-n3c4ccccc4c4cc(-n5c6ccccc6c6ccccc65)ccc43)c2)cc1]

In an embodiment, the fourth compound may be one of Compounds DFD1 to DFD29:

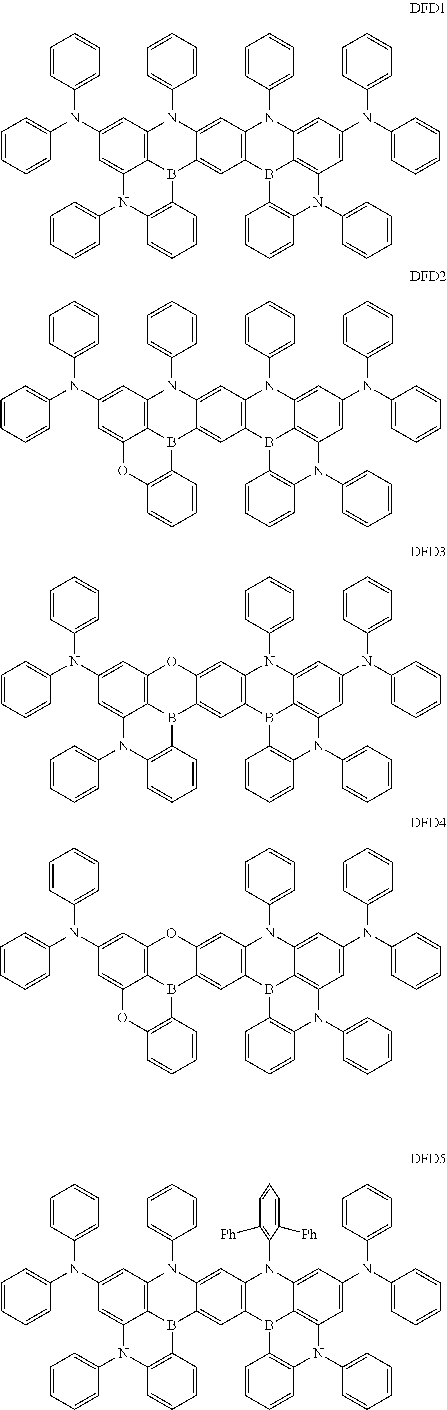

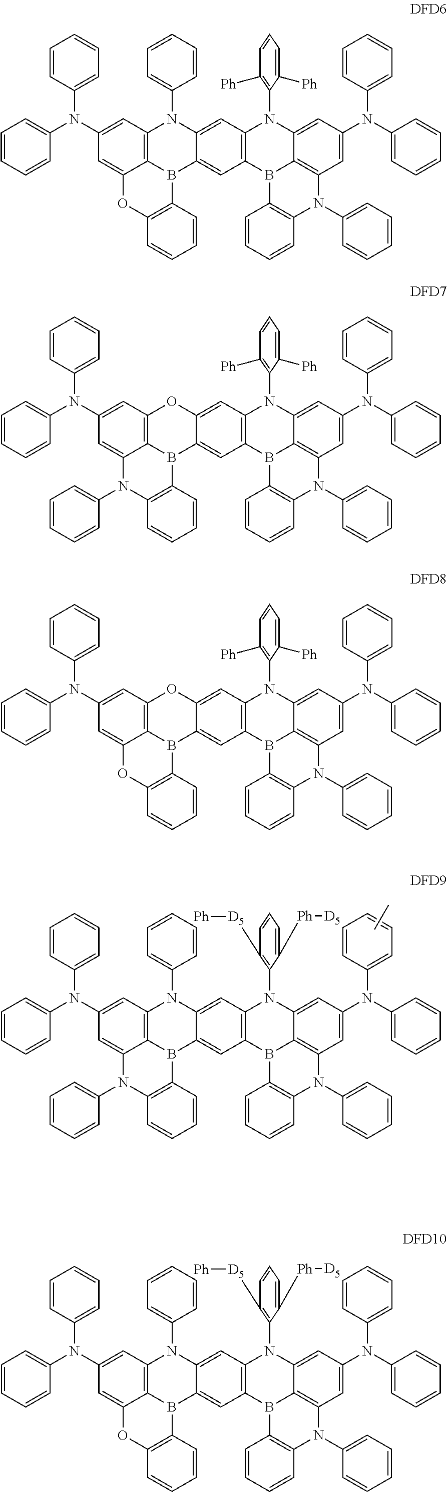

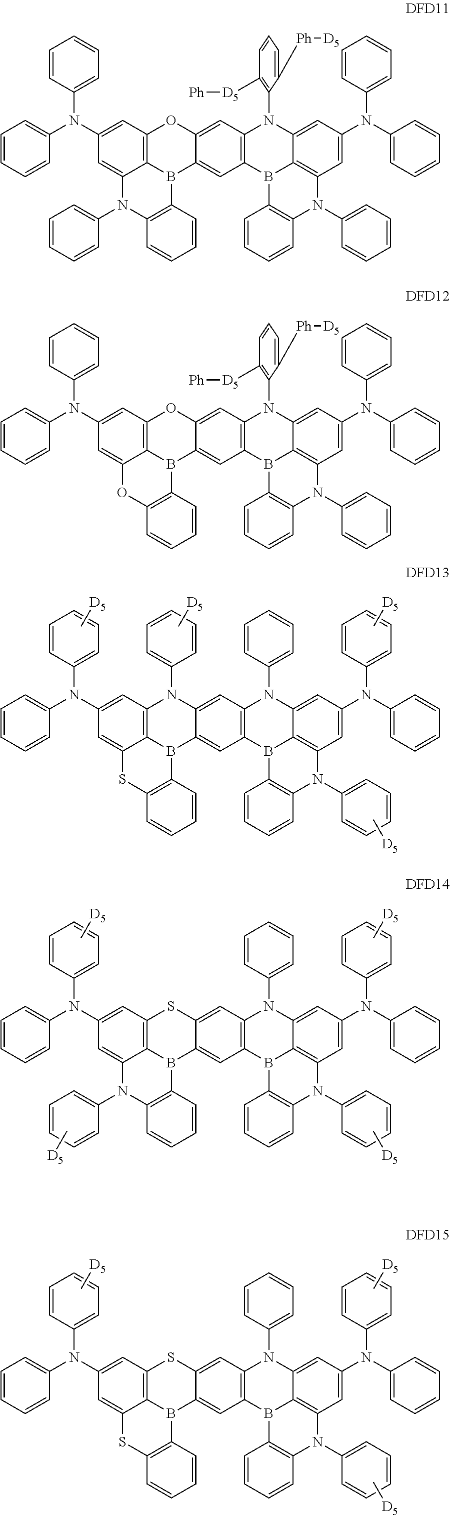

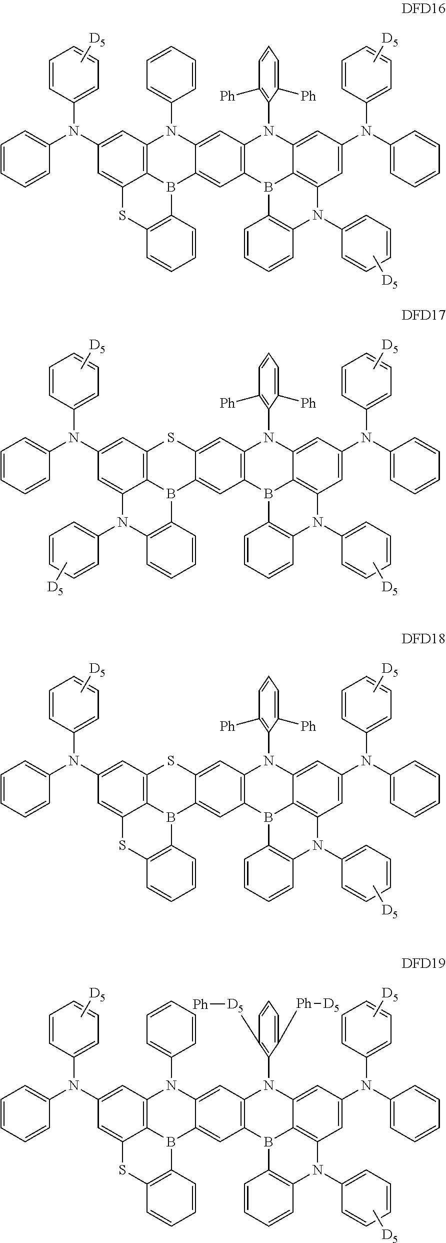

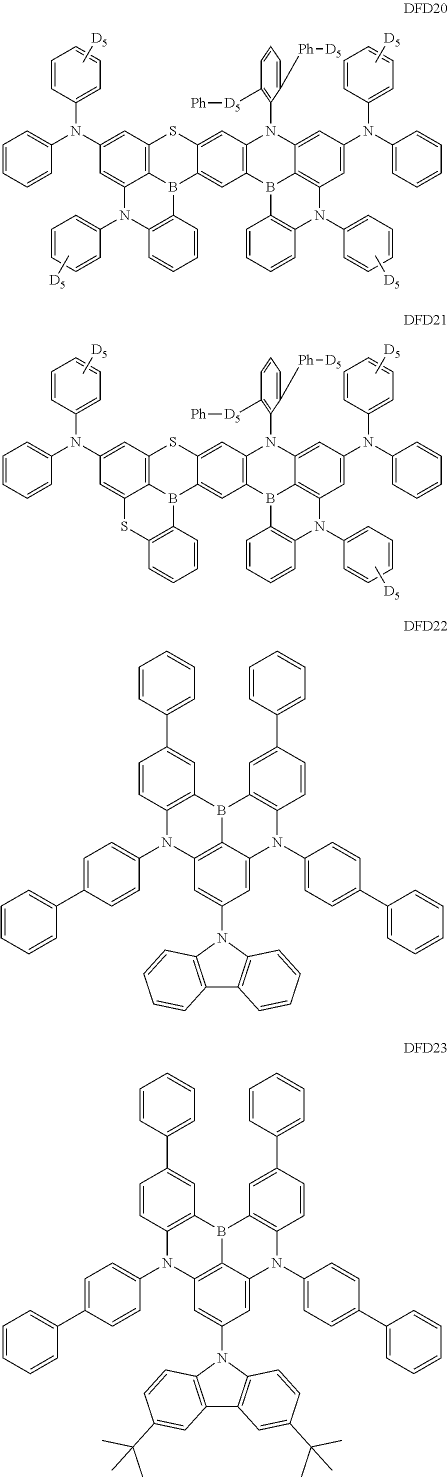

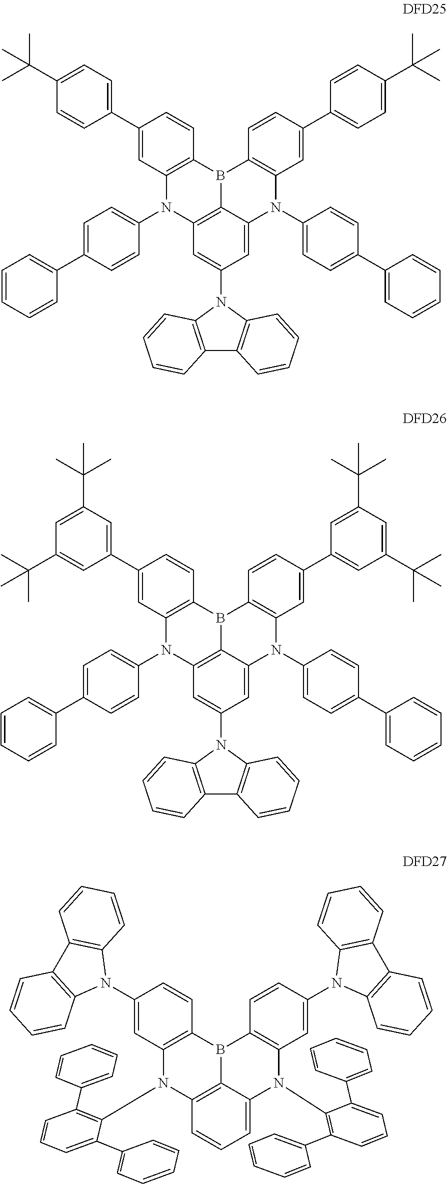 [SMILES: CC(C)(C)c1ccc(-c2ccc3c(c2)N(c2ccc(-c4ccccc4)cc2)c2cc(-n4c5ccccc5c5ccccc54)cc4c2B3c2ccc(-c3ccc(C(C)(C)C)cc3)cc2N4c2ccc(-c3ccccc3)cc2)cc1 ; CC(C)(C)c1cc(-c2ccc3c(c2)N(c2ccc(-c4ccccc4)cc2)c2cc(-n4c5ccccc5c5ccccc54)cc4c2B3c2ccc(-c3cc(C(C)(C)C)cc(C(C)(C)C)c3)cc2N4c2ccc(-c3ccccc3)cc2)cc(C(C)(C)C)c1 ; c1ccc(-c2cccc(-c3ccccc3)c2N2c3cc(-n4c5ccccc5c5ccccc54)ccc3B3c4ccc(-n5c6ccccc6c6ccccc65)cc4N(c4c(-c5ccccc5)cccc4-c4ccccc4)c4cccc2c43)cc1]

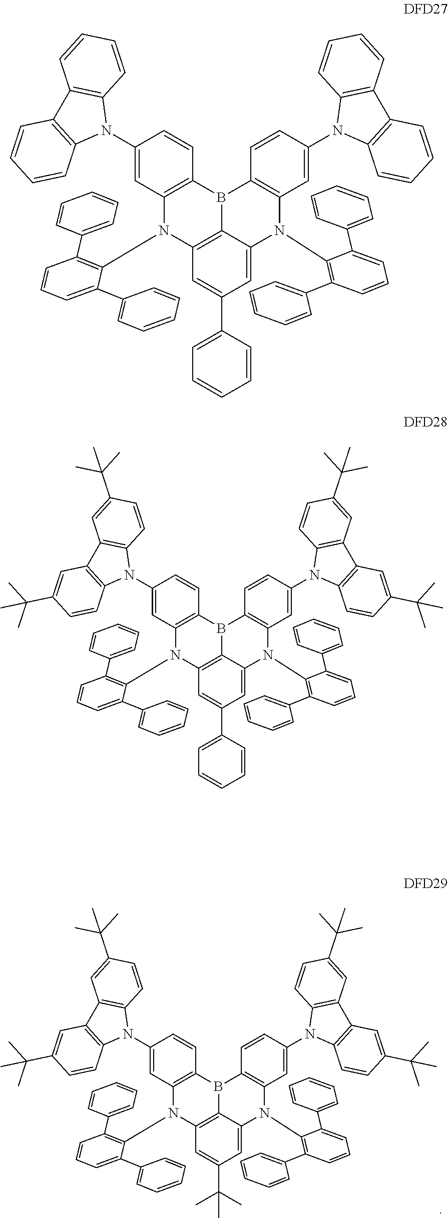

In an embodiment, the fifth compound may include one of Compounds PD1 to PD39, or any combination thereof:

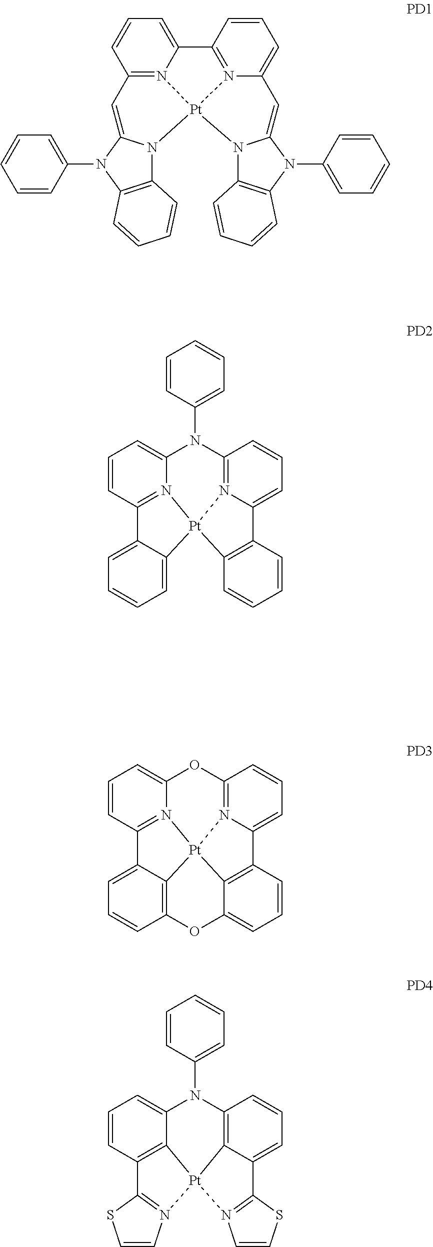

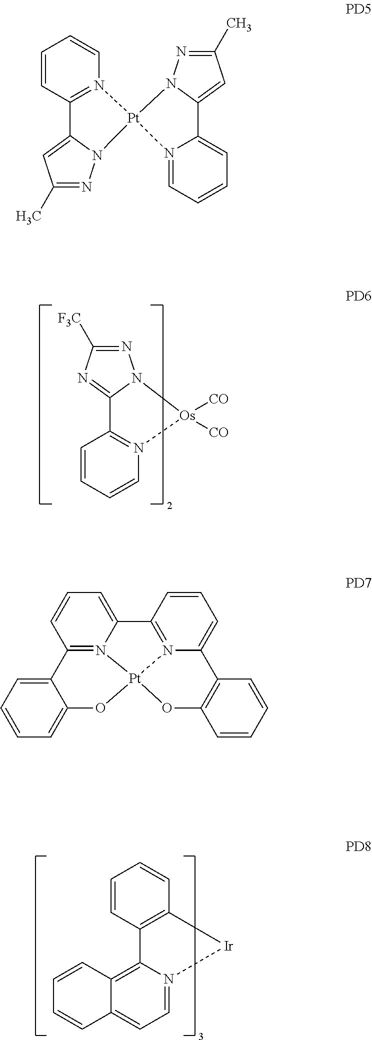

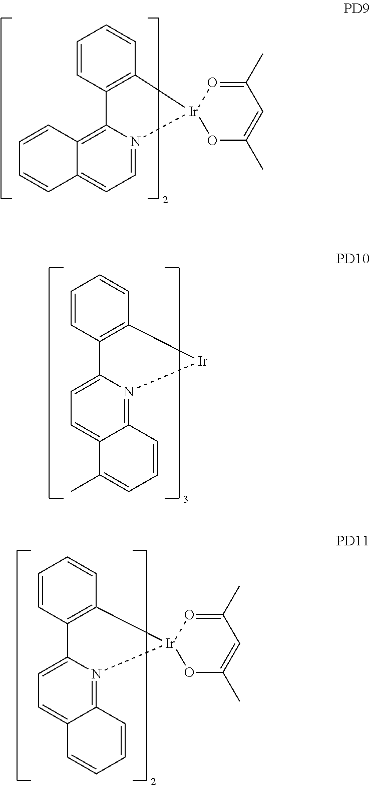

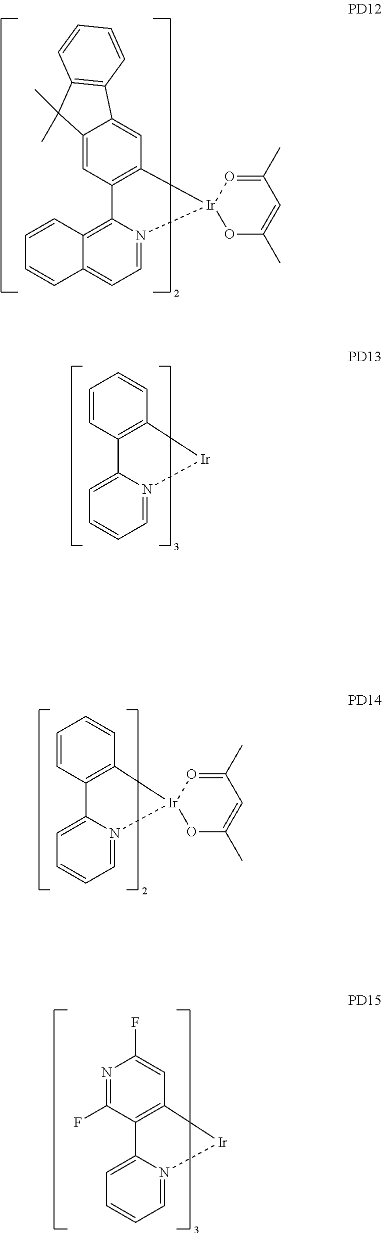

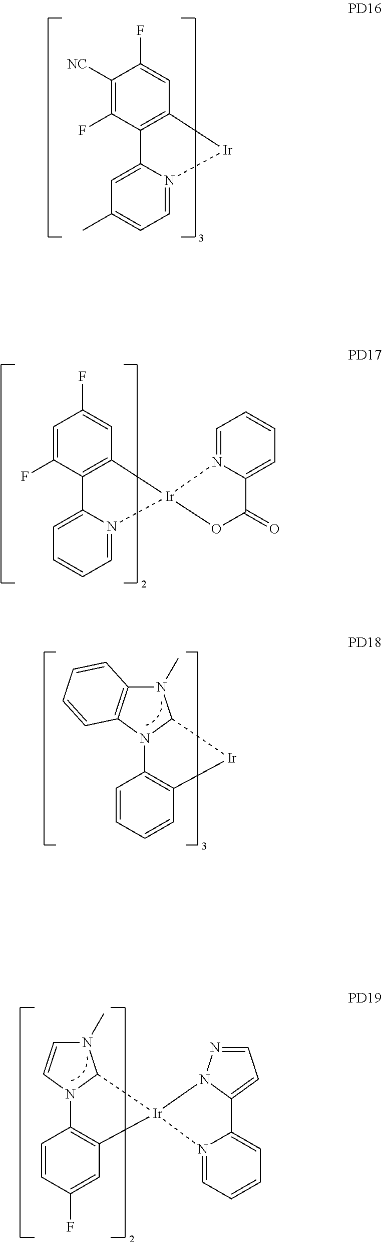

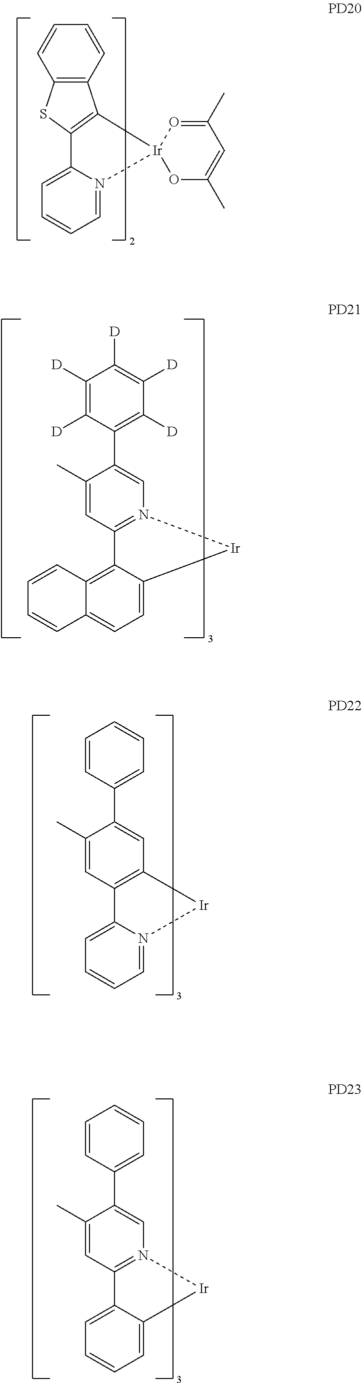

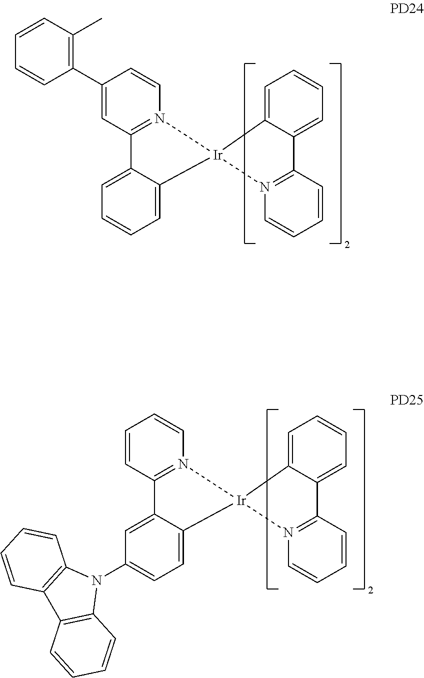

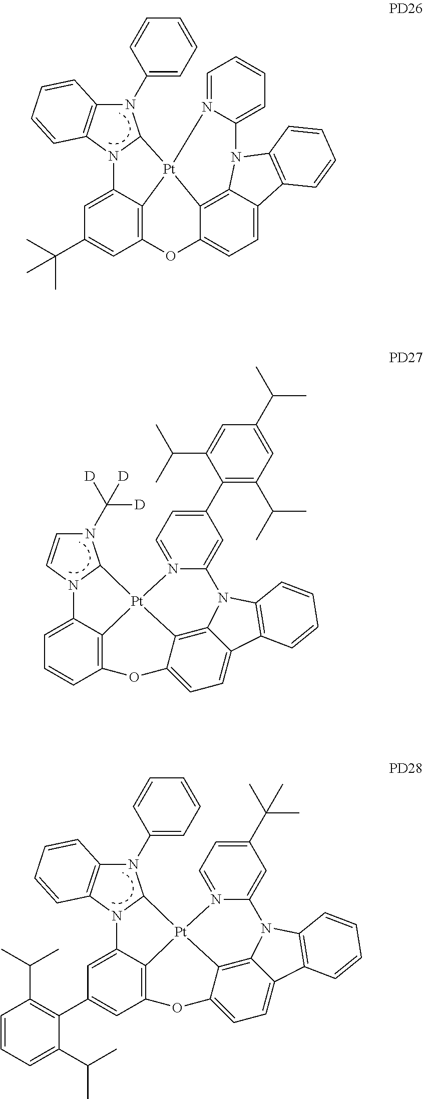

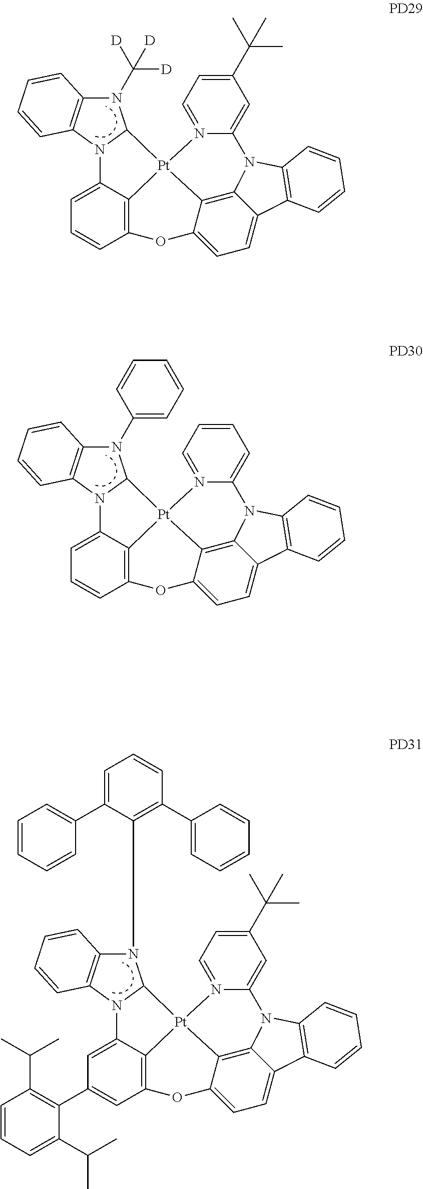

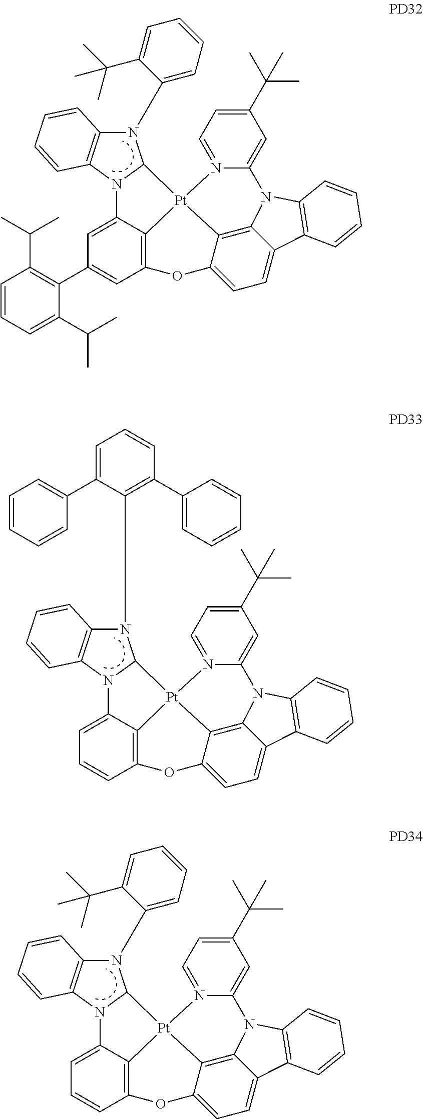

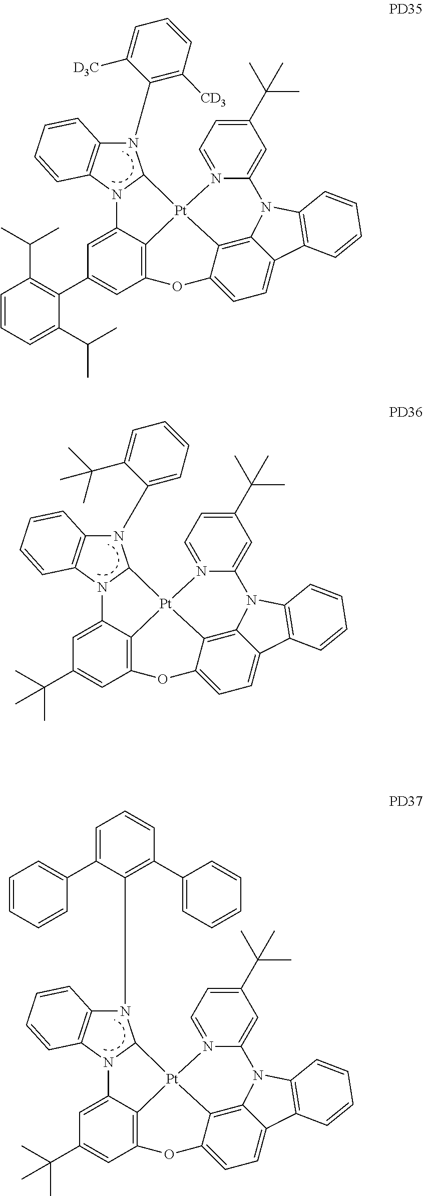

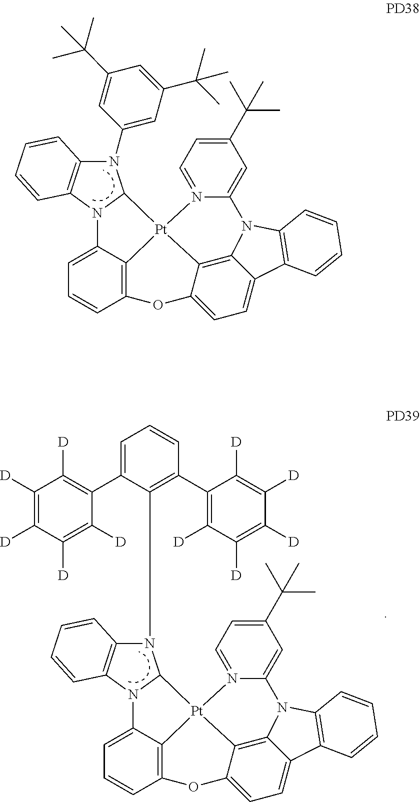

In the compounds described above, Ph represents a phenyl group, D₅ represents substitution with five deuterium, and D₄ represents substitution with four deuterium. For example, a group represented by

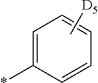

may be identical to a group represented by

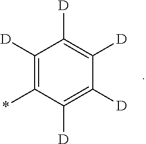 [SMILES: *c1c([2H])c([2H])c([2H])c([2H])c1[2H]]

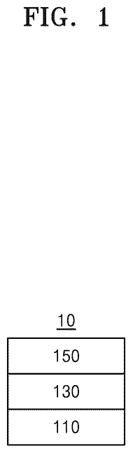

### [Description of FIG. 1]

FIG. 1 is a schematic cross-sectional view of a light-emitting device 10 according to an embodiment. The light-emitting device 10 includes a first electrode 110, an interlayer 130, and a second electrode 150.

Hereinafter, the structure of the light-emitting device 10 according to an embodiment and a method of manufacturing the light-emitting device 10 will be described with reference to FIG. 1.

### [First Electrode 110]

In FIG. 1, a substrate may be further included under the first electrode 110 or on the second electrode 150. In an embodiment, the substrate may be a glass substrate or a plastic substrate. In an embodiment, the substrate may be a flexible substrate, and may include plastics with excellent heat resistance and durability, such as polyimide, polyethylene terephthalate (PET), polycarbonate, polyethylene naphthalate, polyarylate (PAR), polyetherimide, or any combination thereof.

The first electrode 110 may be formed by, for example, depositing or sputtering a material for forming the first electrode 110 on the substrate. When the first electrode 110 is an anode, a material for forming the first electrode 110 may be a high-work function material that facilitates the injection of holes.

The first electrode 110 may be a reflective electrode, a semi-transmissive electrode, or a transmissive electrode. In an embodiment, when the first electrode 110 is a transmissive electrode, a material for forming the first electrode 110 may include indium tin oxide (ITO), indium zinc oxide (IZO), tin oxide (SnO₂), zinc oxide (ZnO), or any combination thereof. In embodiments, when the first electrode 110 is a semi-transmissive electrode or a reflective electrode, a material for forming the first electrode 110 may include magnesium (Mg), silver (Ag), aluminum (AI), aluminum-lithium (Al—Li), calcium (Ca), magnesium-indium (Mg—In), magnesium-silver (Mg—Ag), or any combination thereof.

The first electrode 110 may have a structure consisting of a single layer or a structure including multiple layers. For example, the first electrode 110 may have a three-layer structure of ITO/Ag/ITO.

### [Interlayer 130]

The interlayer 130 may be arranged on the first electrode 110. The interlayer 130 may include an emission layer.

The interlayer 130 may further include a hole transport region between the first electrode 110 and the emission layer, and an electron transport region between the emission layer and the second electrode 150.

The interlayer 130 may further include, in addition to various organic materials, a metal-containing compound, an inorganic material such as quantum dots, or the like.

In an embodiment, the interlayer 130 may include two or more emitting units stacked between the first electrode 110 and the second electrode 150, and at least one charge generation layer between the two or more emitting units. When the interlayer 130 includes the two or more emitting units and the at least one charge generation layer, the light-emitting device 10 may be a tandem light-emitting device.

### [Hole Transport Region in Interlayer 130]

The hole transport region may have a structure consisting of a layer consisting of a single material, a structure consisting of a layer including different materials, or a structure including multiple layers including different materials.

The hole transport region may include a hole injection layer, a hole transport layer, an emission auxiliary layer, an electron blocking layer, or any combination thereof.

In embodiment, the hole transport region may have a multi-layer structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron blocking layer structure, wherein the layers of each structure may be stacked from the first electrode 110 in its respective stated order, but the structure of the hole transport region is not limited thereto.

The hole transport region may include a compound represented by Formula 201, a compound represented by Formula 202, or any combination thereof:

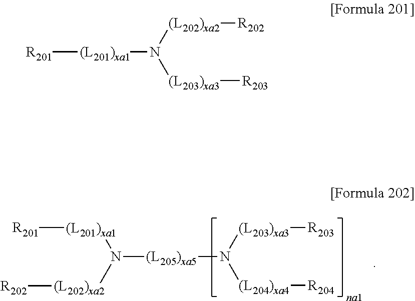

In Formulae 201 and 202,
L₂₀₁ to L₂₀₄ may each independently be a C₅-C₆₀ carbocyclic group unsubstituted or substituted with at least one R₁₀ₐ or a C₁-C₆₀ heterocyclic group unsubstituted or substituted with at least one R₁₀ₐ,
L₂₀₅ may be *—O—*′, *—S—*′, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
xa1 to xa4 may each independently be an integer from 0 to 5,
xa5 may be an integer from 1 to 10,
R₂₀₁ to R₂₀₄ and Q₂₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
R₂₀₁ and R₂₀₂ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group (e.g., a carbazole group or the like) unsubstituted or substituted with at least one R₁₀ₐ (e.g., see Compound HT16),
R₂₀₃ and R₂₀₄ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group unsubstituted or substituted with at least one R₁₀ₐ, and
na1 may be an integer from 1 to 4.

In embodiments, the compound represented by Formula 201 and the compound represented by Formula 202 may each independently include at least one of groups represented by Formulae CY201 to CY217:

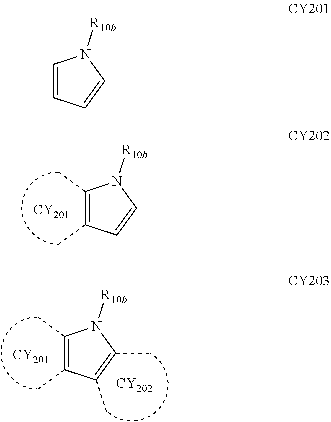

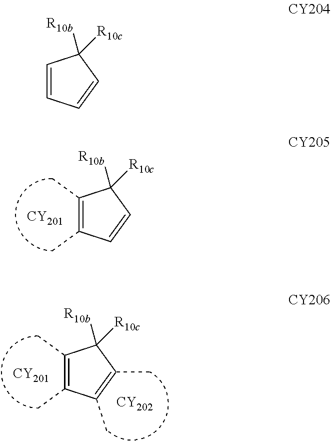

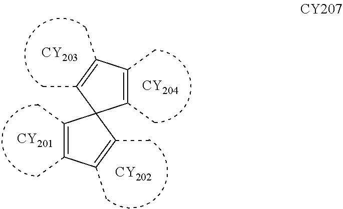

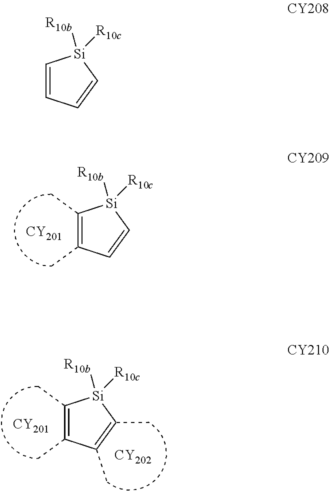

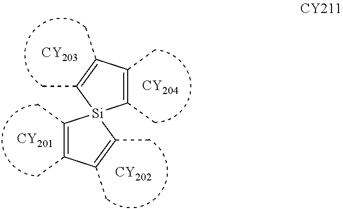

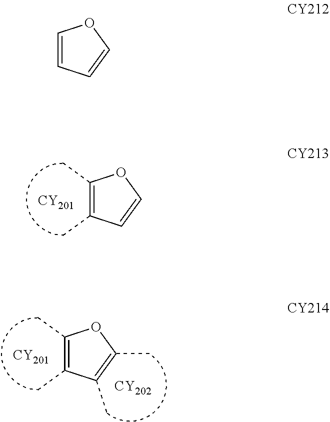

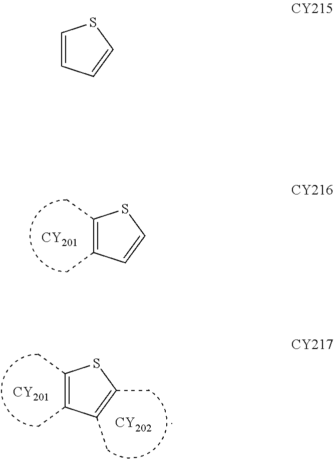

In Formulae CY201 to CY217, R_{10b} and R_{10c} may each independently be the same as described in connection with R₁₀ₐ, ring CY201 to ring CY204 may each independently be a C₃-C₂₀ carbocyclic group or a C₁-C₂₀ heterocyclic group, and at least one hydrogen in Formulae CY201 to CY217 may be unsubstituted or substituted with R₁₀ₐ.

In embodiments, ring CY₂₀₁ to ring CY₂₀₄ in Formulae CY201 to CY217 may each independently be a benzene group, a naphthalene group, a phenanthrene group, or an anthracene group.

In embodiments, the compound represented by Formula 201 and the compound represented by Formula 202 may each independently include at least one of groups represented by Formulae CY201 to CY203.

In embodiments, the compound represented by Formula 201 may include at least one of groups represented by Formulae CY201 to CY203 and at least one of groups represented by Formulae CY204 to CY217.

In embodiments, in Formula 201, xa1 may be 1, R₂₀₁ may be a group represented by one of Formulae CY201 to CY203, xa2 may be 0, and R₂₀₂ may be one of the groups represented by Formulae CY204 to CY207.

In embodiments, the compound represented by Formula 201 and the compound represented by Formula 202 may each not include groups represented by Formulae CY201 to CY203.

In embodiments, the compound represented by Formula 201 and the compound represented by Formula 202 may each not include groups represented by Formulae CY201 to CY203, and may each independently include at least one of groups represented by Formulae CY204 to CY217.

In embodiments, the compound represented by Formula 201 and the compound represented by Formula 202 may each not include a group represented by one of Formulae CY201 to CY217.

In an embodiment, the hole transport region may include: one of Compounds HT1 to HT46; m-MTDATA; TDATA; 2-TNATA; NPB(NPD); β-NPB; TPD; spiro-TPD; spiro-NPB; methylated NPB; TAPC; HMTPD; 4,4′,4″-tris(N-carbazolyl)triphenylamine (TCTA); polyaniline/dodecylbenzenesulfonic acid (PANI/DBSA); poly(3,4-ethylenedioxythiophene)/poly(4-styrenesulfonate) (PEDOT/PSS); polyaniline/camphor sulfonic acid (PANI/CSA); polyaniline/poly(4-styrenesulfonate) (PANI/PSS); or any combination thereof:

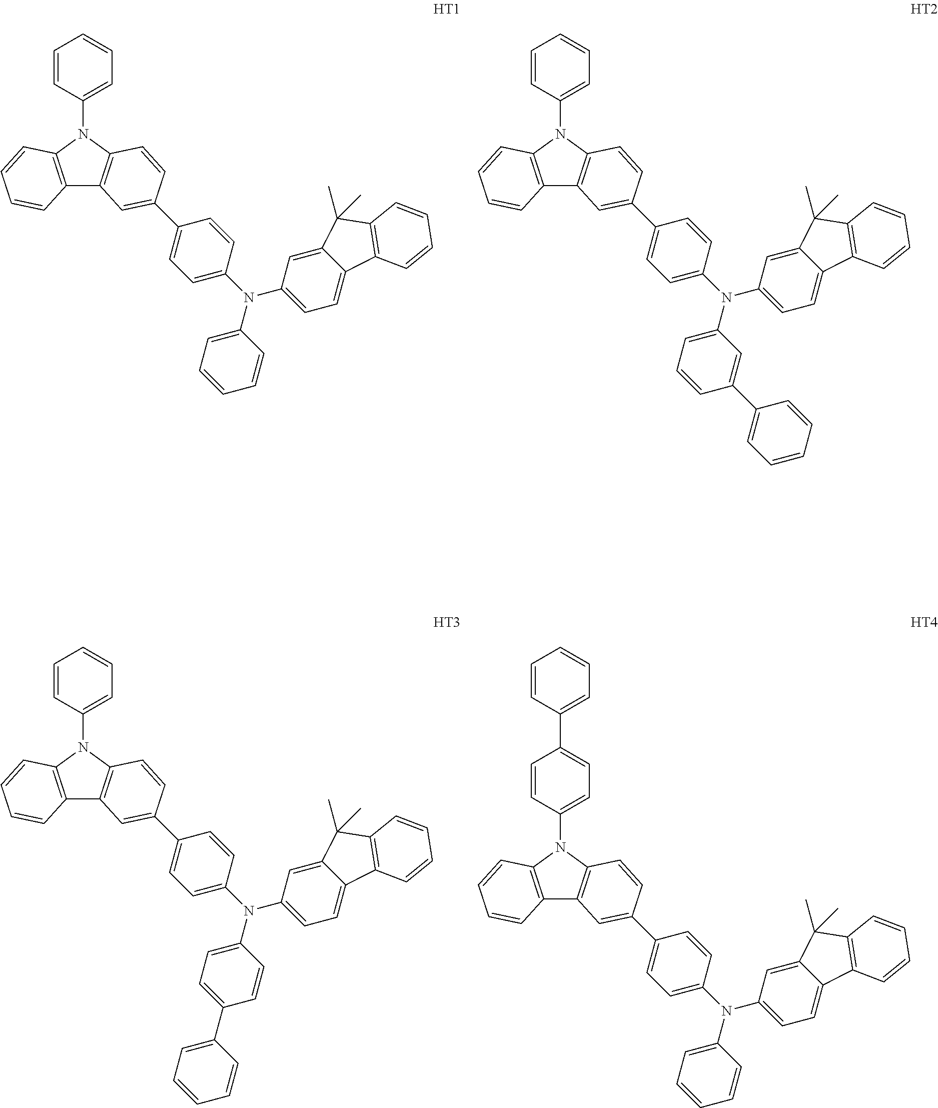 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccccc3)c3ccc(-c4ccc5c(c4)c4ccccc4n5-c4ccc(-c5ccccc5)cc4)cc3)cc21]

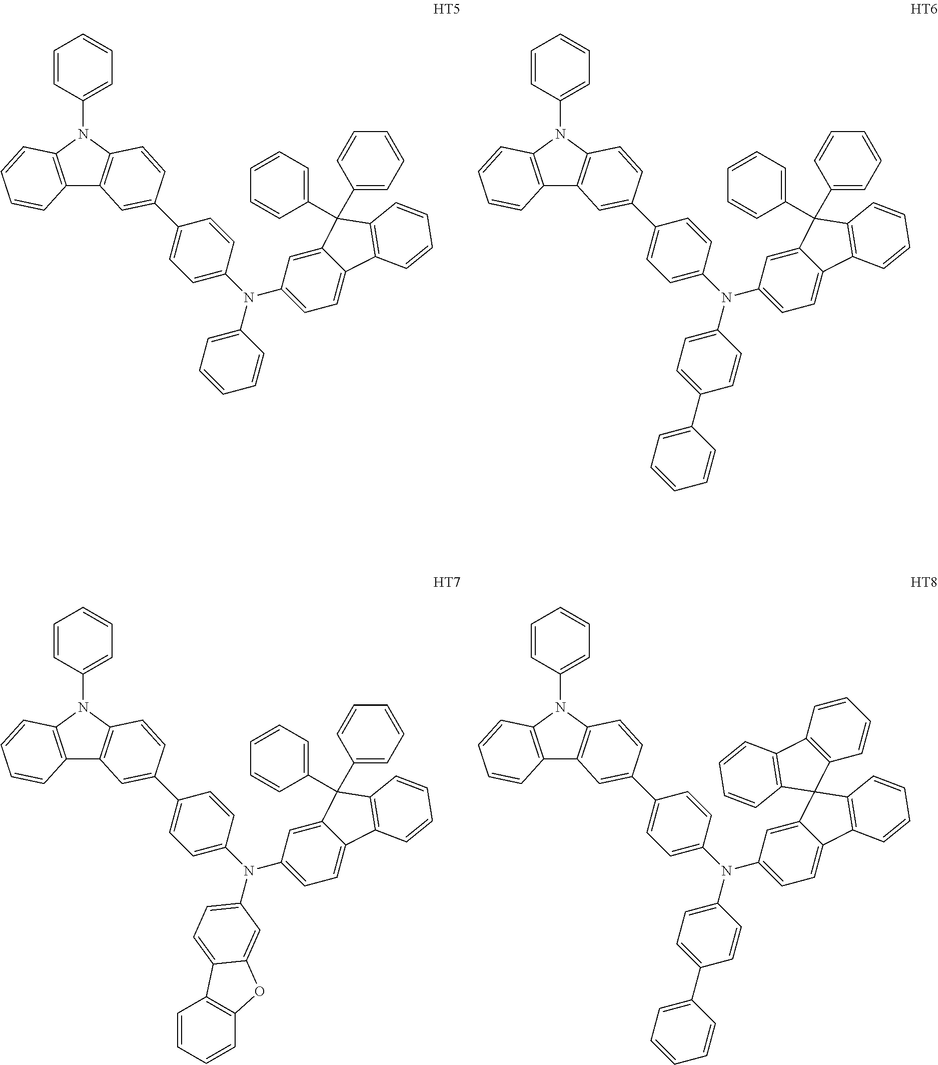 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

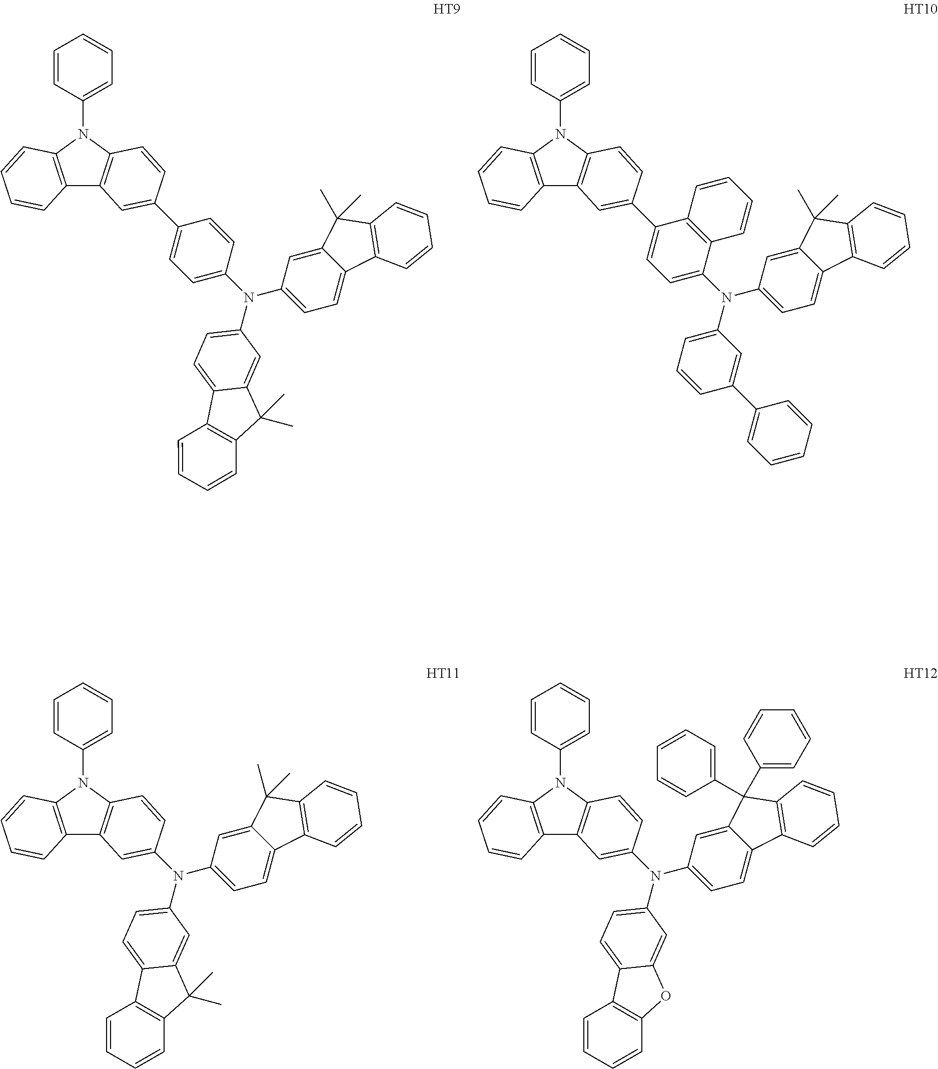 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

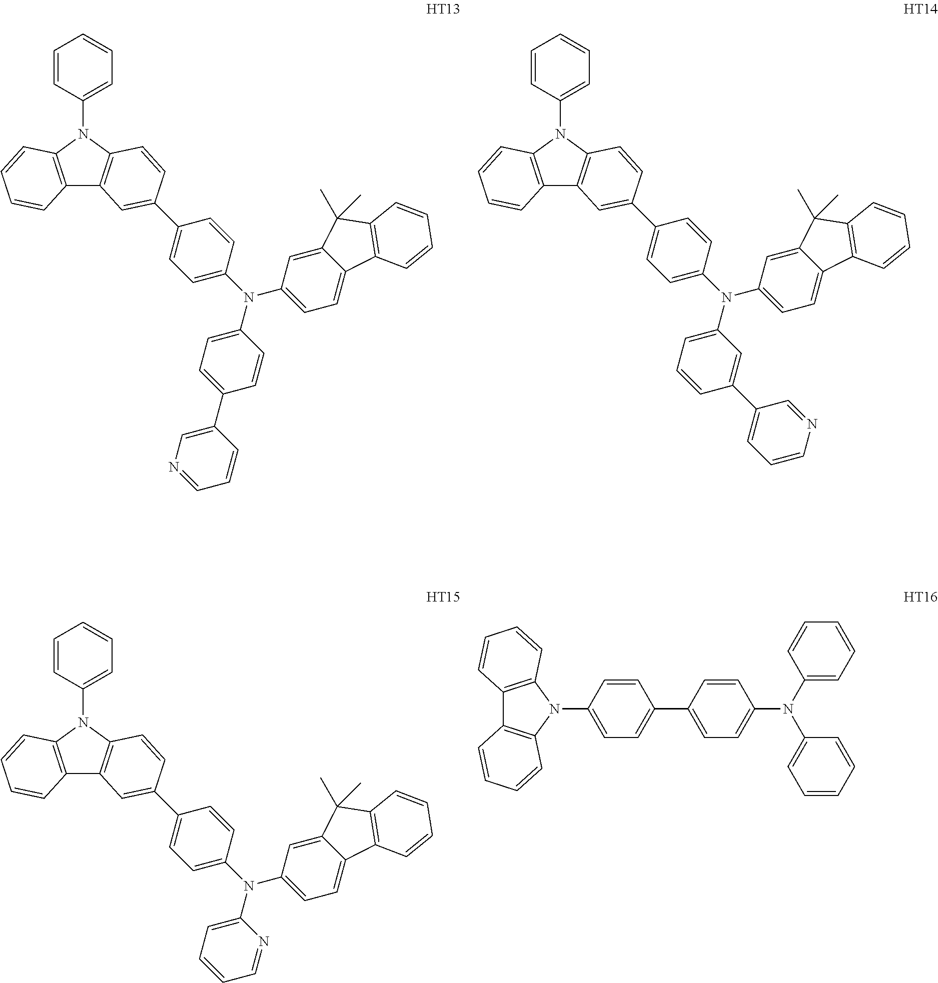 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1]

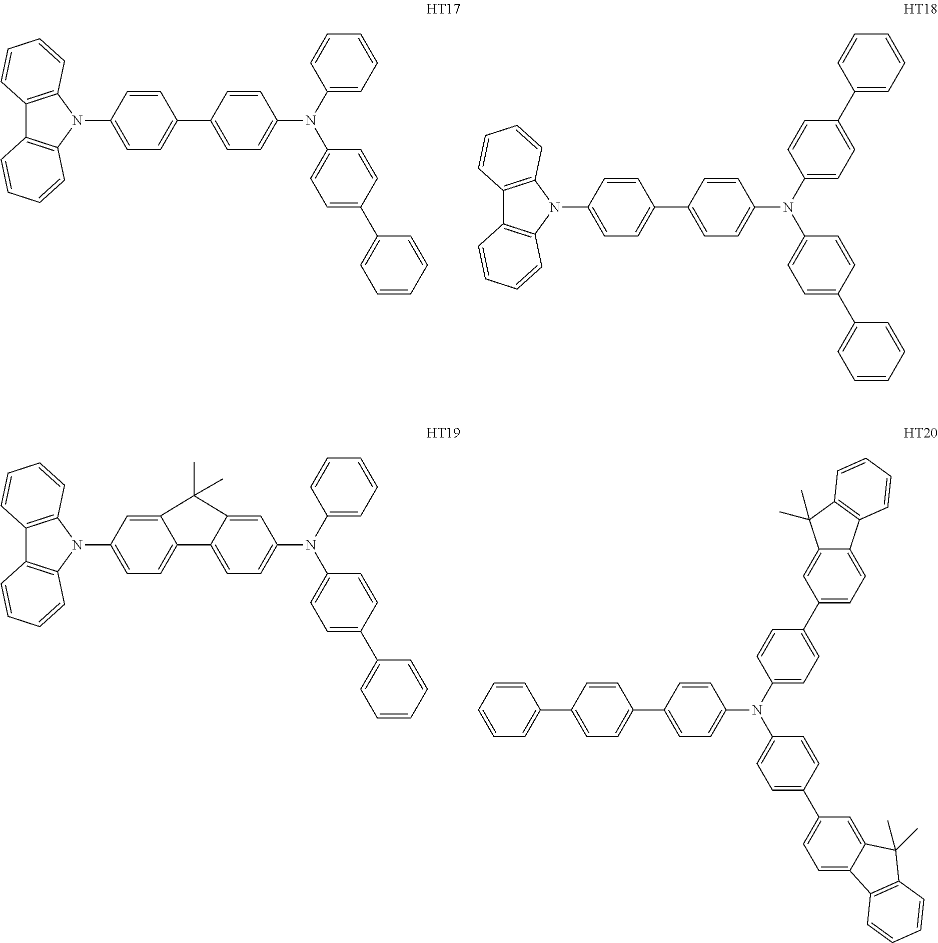 [SMILES: c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21]

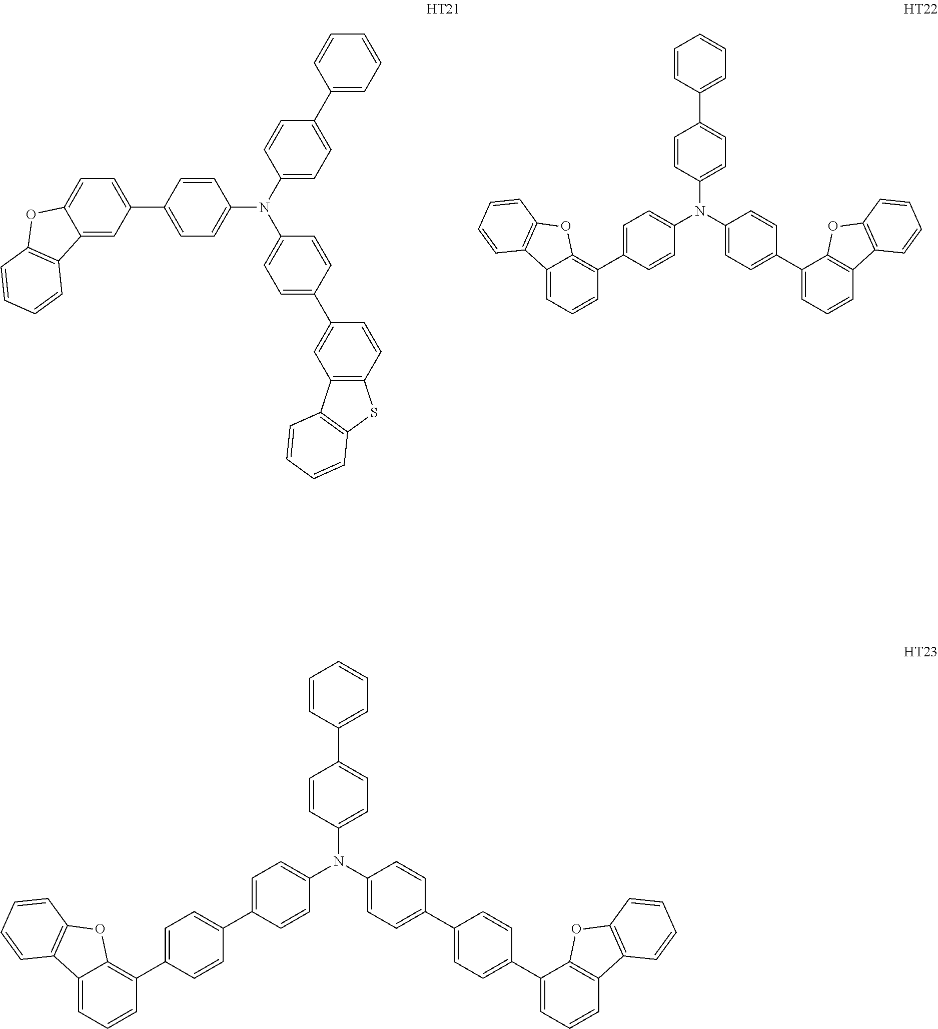 [SMILES: c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1]

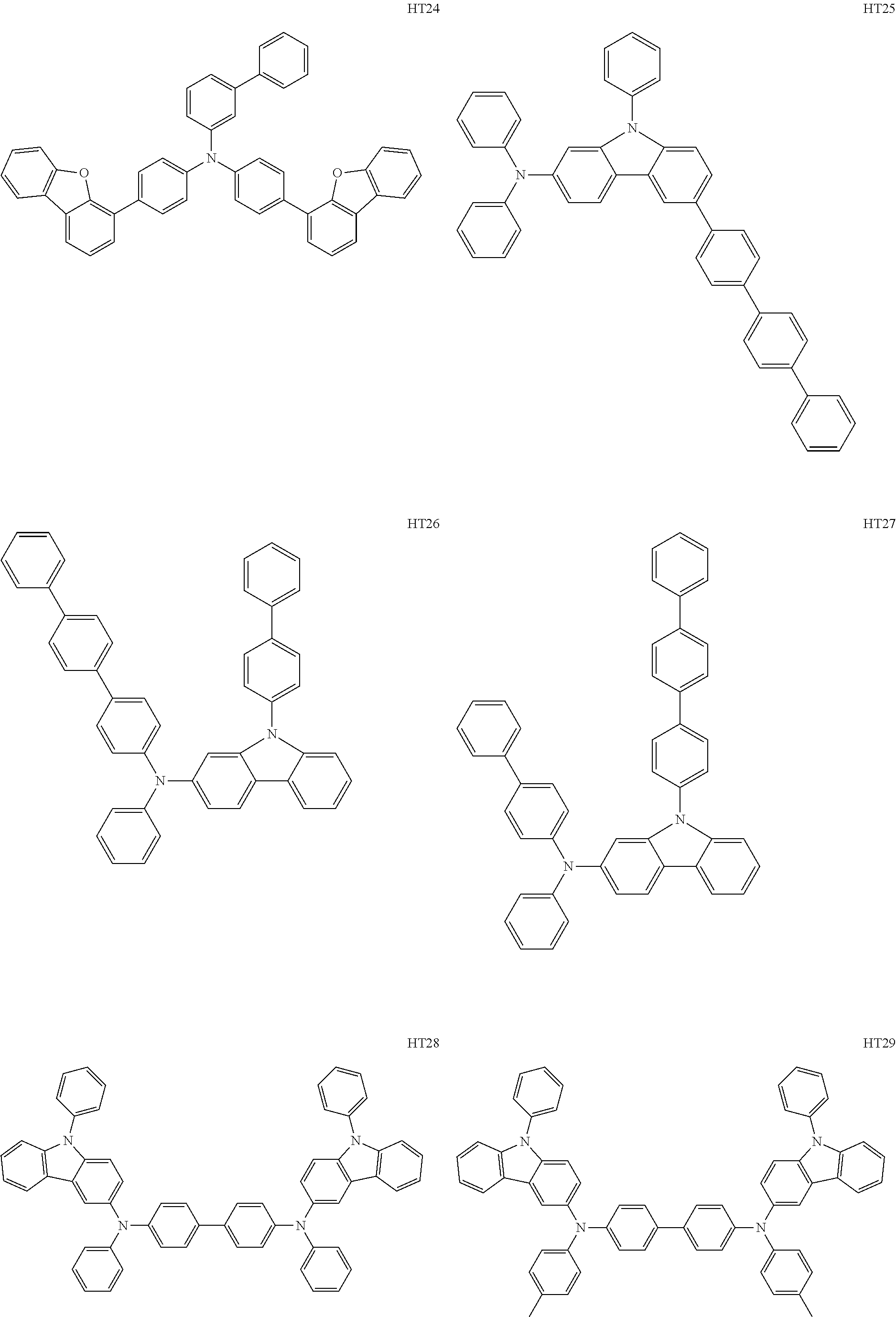

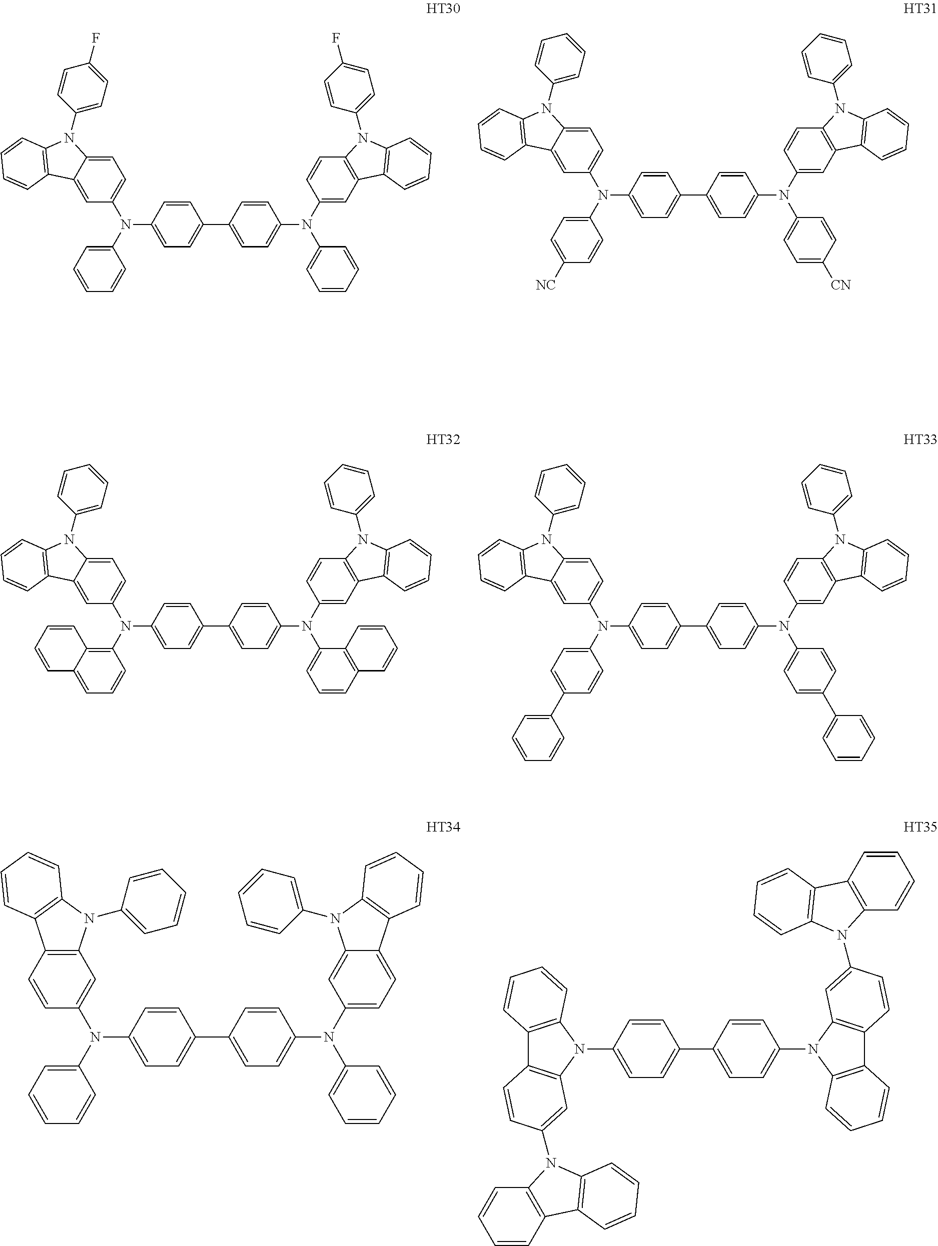

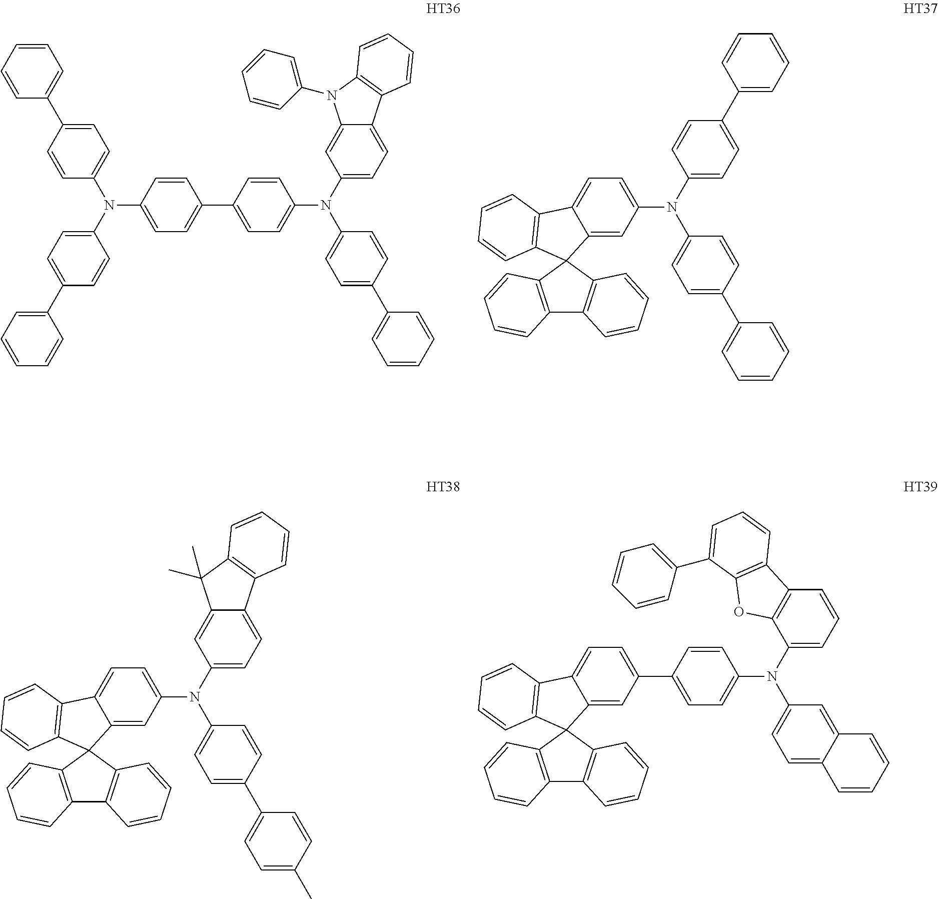 [SMILES: c1ccc(-c2ccc(N(c3ccc(-c4ccccc4)cc3)c3ccc(-c4ccc(N(c5ccc(-c6ccccc6)cc5)c5ccc6c7ccccc7n(-c7ccccc7)c6c5)cc4)cc3)cc2)cc1 ; c1ccc(-c2ccc(N(c3ccc(-c4ccccc4)cc3)c3ccc4c(c3)C3(c5ccccc5-c5ccccc53)c3ccccc3-4)cc2)cc1 ; Cc1ccc(-c2ccc(N(c3ccc4c(c3)C(C)(C)c3ccccc3-4)c3ccc4c(c3)C3(c5ccccc5-c5ccccc53)c3ccccc3-4)cc2)cc1 ; c1ccc(-c2cccc3c2oc2c(N(c4ccc(-c5ccc6c(c5)C5(c7ccccc7-c7ccccc75)c5ccccc5-6)cc4)c4ccc5ccccc5c4)cccc23)cc1]

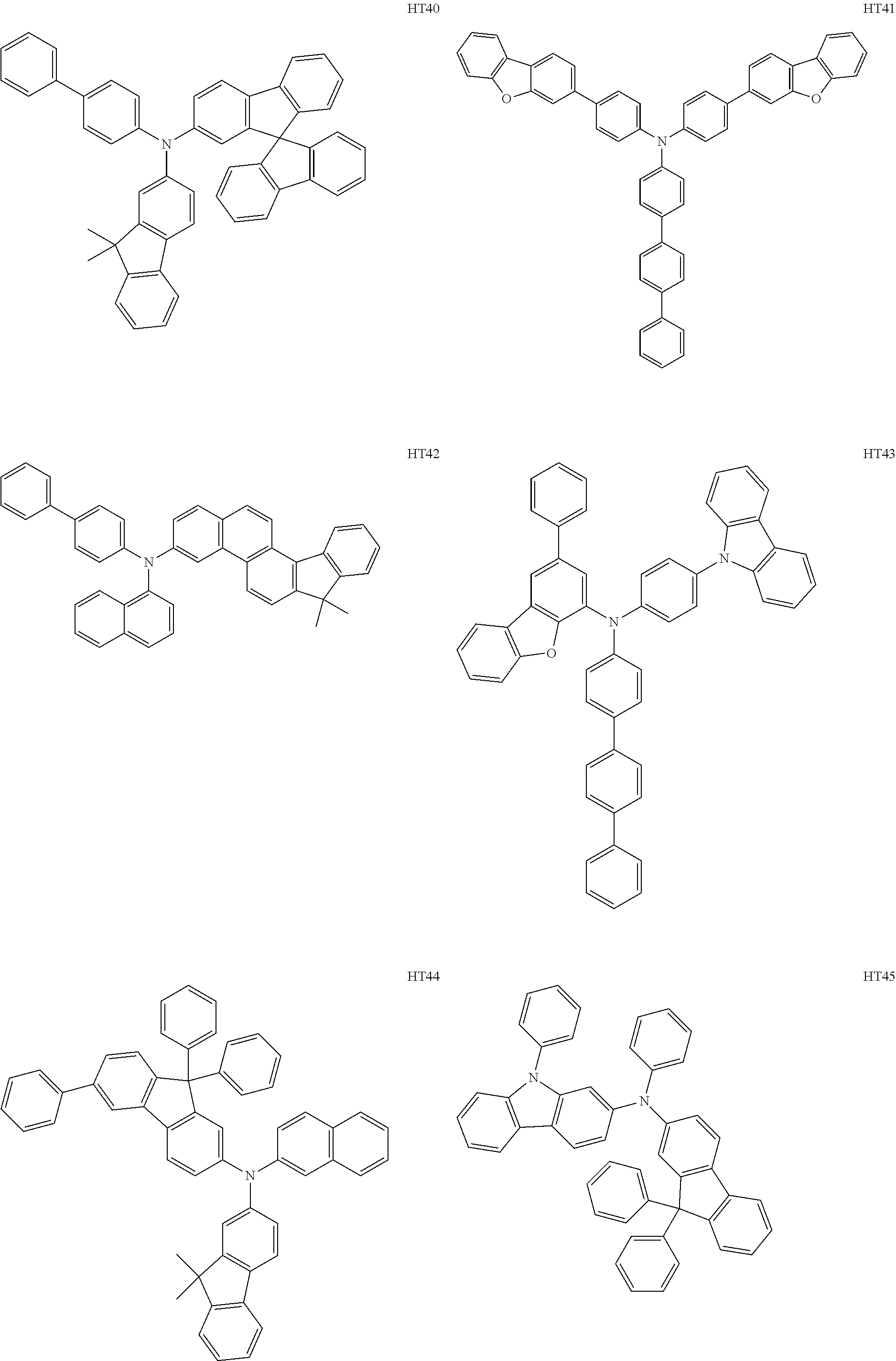

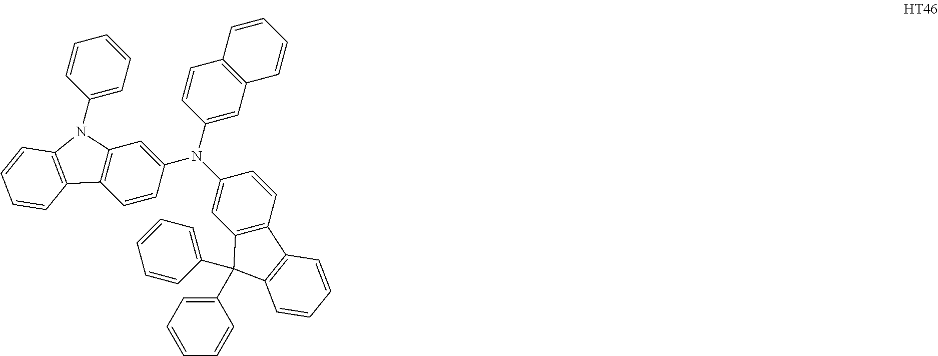 [SMILES: c1ccc(-n2c3ccccc3c3ccc(N(c4ccc5c(c4)C(c4ccccc4)(c4ccccc4)c4ccccc4-5)c4ccc5ccccc5c4)cc32)cc1]

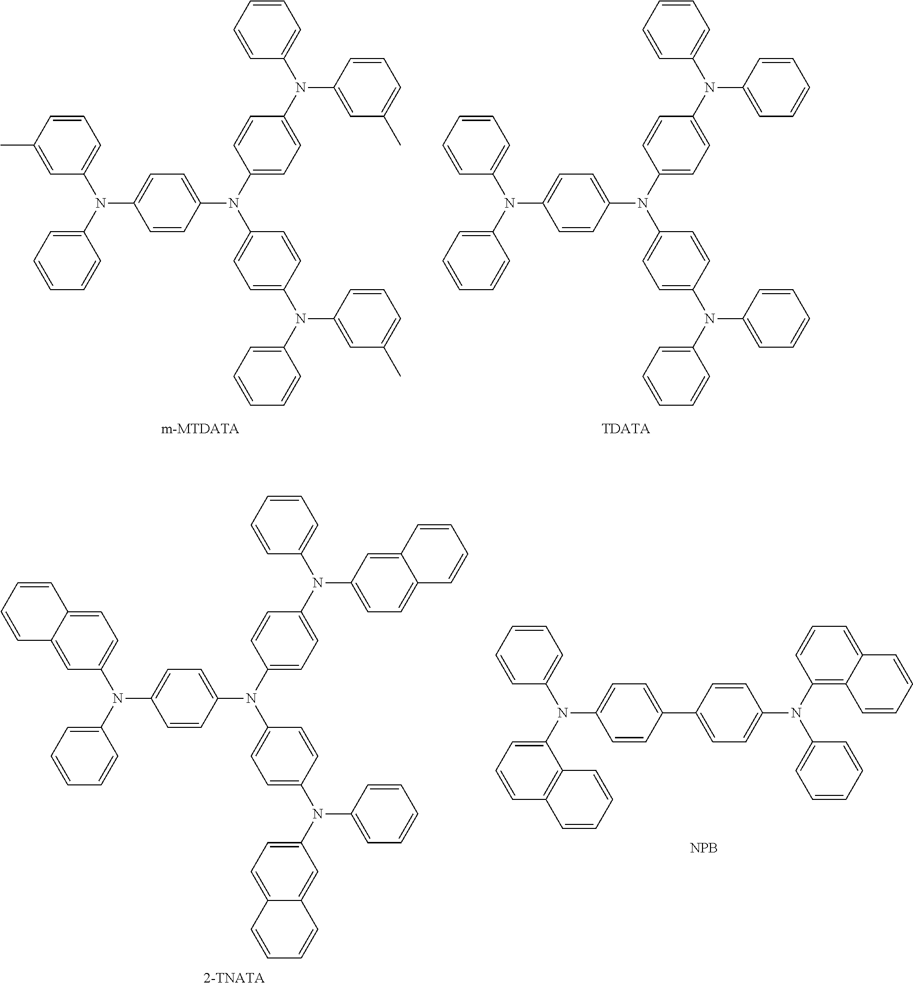

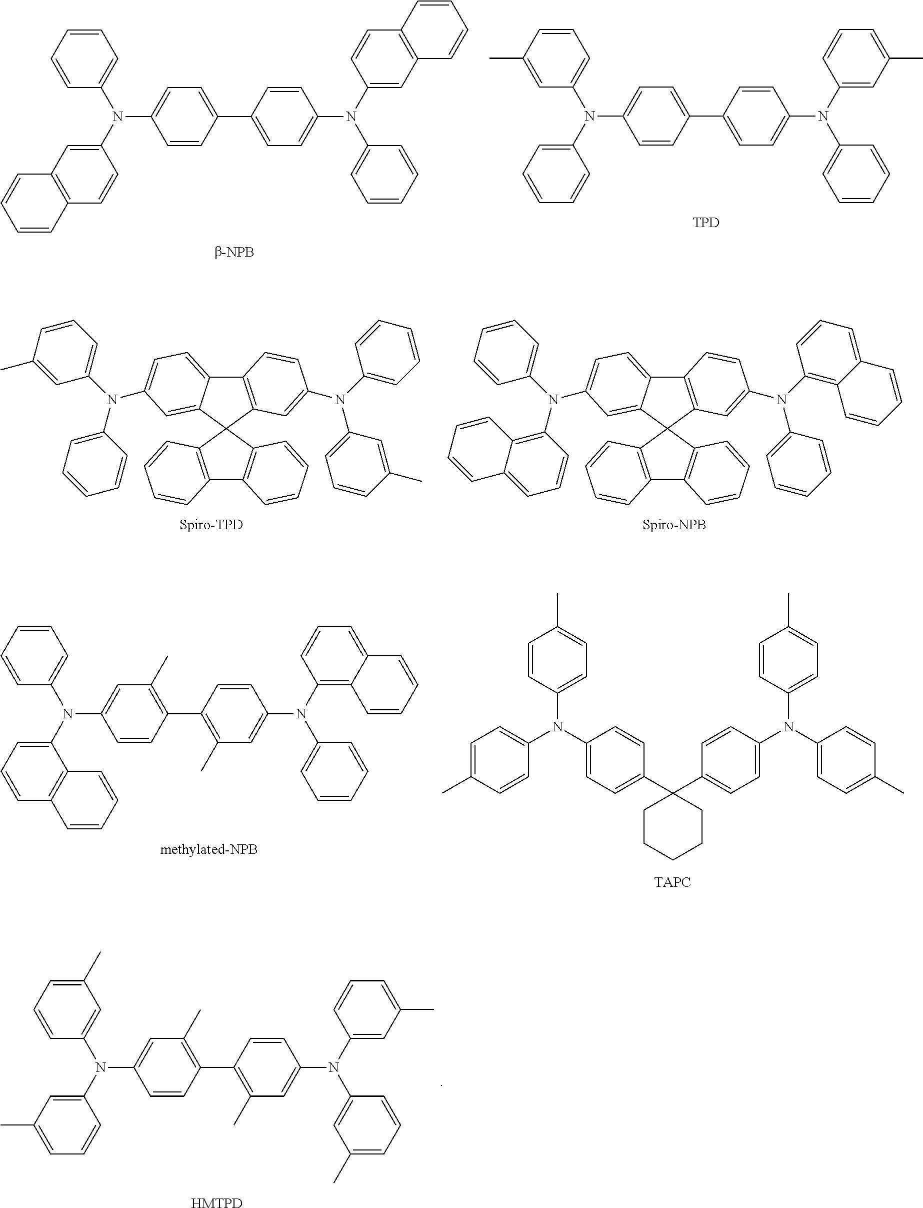

A thickness of the hole transport region may be in a range of about 50 Å to about 10,000 Å. For example, the thickness of the hole transport region may be in a range of about 100 Å to about 4,000 Å. When the hole transport region includes a hole injection layer, a hole transport layer, or any combination thereof, a thickness of the hole injection layer may be in a range of about 100 Å to about 9,000 Å, and a thickness of the hole transport layer may be in a range of about 50 Å to about 2,000 Å. For example, the thickness of the hole injection layer may be in a range of about 100 Å to about 1,000 Å. For example, the thickness of the hole transport layer may be in a range of about 100 Å to about 1,500 Å. When the thicknesses of the hole transport region, the hole injection layer, and the hole transport layer are within these ranges, satisfactory hole transporting characteristics may be obtained without a substantial increase in driving voltage.

The emission auxiliary layer may increase light-emission efficiency by compensating for an optical resonance distance according to a wavelength of light emitted by the emission layer, and the electron blocking layer may block the leakage of electrons from an emission layer to a hole transport region. Materials that may be included in the hole transport region may be included in the emission auxiliary layer and the electron blocking layer.

[p-dopant]

The hole transport region may further include, in addition to these materials, a charge-generation material for the improvement of conductive properties. The charge-generation material may be uniformly or non-uniformly dispersed in the hole transport region (for example, in the form of a single layer consisting of a charge-generation material).

The charge-generation material may be, for example, a p-dopant.

In an embodiment, a lowest unoccupied molecular orbital (LUMO) energy level of the p-dopant may be less than or equal to about −3.5 eV.

In an embodiment, the p-dopant may include a quinone derivative, a cyano group-containing compound, a compound including element EL1 and element EL2, or any combination thereof.

Examples of a quinone derivative may include TCNQ, F4-TCNQ, and the like.

Examples of a cyano group-containing compound may include HAT-CN, a compound represented by Formula 221, and the like:

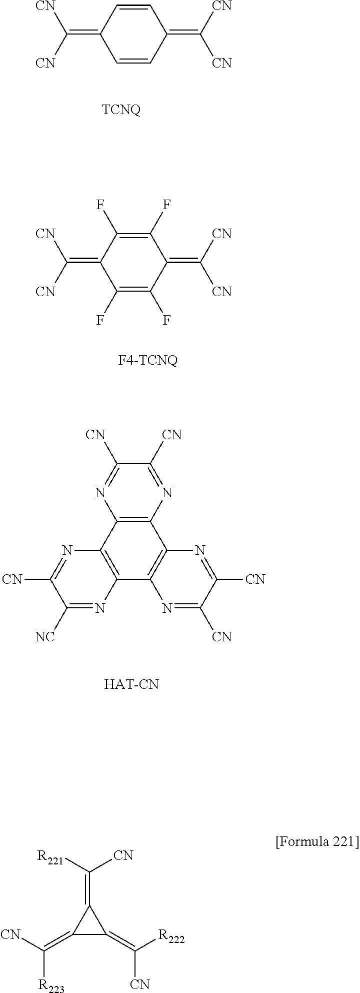

In Formula 221,
R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and
at least one of R₂₂₁ to R₂₂₃ may each independently be a C₃-C₆₀ carbocyclic group or a C₁-C₆₀ heterocyclic group, each substituted with: a cyano group; —F; —Cl; —Br; —I; a C₁-C₂₀ alkyl group substituted with a cyano group, —F, —Cl, —Br, —I, or any combination thereof; or any combination thereof.

In the compound including element EL1 and element EL2, element EL1 may be a metal, a metalloid, or any combination thereof, and element EL2 may be a non-metal, a metalloid, or any combination thereof.

Examples of a metal may include: an alkali metal (for example, lithium (Li), sodium (Na), potassium (K), rubidium (Rb), cesium (Cs), etc.); an alkaline earth metal (for example, beryllium (Be), magnesium (Mg), calcium (Ca), strontium (Sr), barium (Ba), etc.); a transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), etc.); a post-transition metal (for example, zinc (Zn), indium (In), tin (Sn), etc.); a lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), etc.); and the like.

Examples of a metalloid may include silicon (Si), antimony (Sb), tellurium (Te), and the like.

Examples of a non-metal may include oxygen (O), halogen (for example, F, Cl, Br, I, etc.), and the like.

Examples of a compound including element EL1 and element EL2 may include a metal oxide, a metal halide (for example, a metal fluoride, a metal chloride, a metal bromide, a metal iodide, etc.), a metalloid halide (for example, a metalloid fluoride, a metalloid chloride, a metalloid bromide, a metalloid iodide, etc.), a metal telluride, or any combination thereof.

Examples of a metal oxide may include a tungsten oxide (for example, WO, W₂O₃, WO₂, WO₃, W₂O₅, etc.), a vanadium oxide (for example, VO, V₂O₃, VO₂, V₂O₅, etc.), a molybdenum oxide (MoO, Mo₂O₃, MoO₂, MoO₃, Mo₂O₅, etc.), a rhenium oxide (for example, ReO₃, etc.), and the like.

Examples of a metal halide may include an alkali metal halide, an alkaline earth metal halide, a transition metal halide, a post-transition metal halide, a lanthanide metal halide, and the like.

Examples of an alkali metal halide may include LiF, NaF, KF, RbF, CsF, LiCl, NaCl, KCl, RbCl, CsCl, LiBr, NaBr, KBr, RbBr, CsBr, LiI, NaI, KI, RbI, CsI, and the like.

Examples of an alkaline earth metal halide may include BeF₂, MgF₂, CaF₂, SrF₂, BaF₂, BeCl₂, MgCl₂, CaCl₂, SrCl₂, BaCl₂, BeBr₂, MgBr₂, CaBr₂, SrBr₂, BaBr₂, BeI₂, MgI₂, CaI₂, SrI₂, BaI₂, and the like.

Examples of an transition metal halide may include a titanium halide (for example, TiF₄, TiCl₄, TiBr₄, TiI₄, etc.), a zirconium halide (for example, ZrF₄, ZrCl₄, ZrBr₄, ZrI₄, etc.), a hafnium halide (for example, HfF₄, HfCl₄, HfBr₄, HfI₄, etc.), a vanadium halide (for example, VF₃, VCl₃, VBr₃, VI₃, etc.), a niobium halide (for example, NbF₃, NbCl₃, NbBr₃, NbI₃, etc.), a tantalum halide (for example, TaF₃, TaCl₃, TaBr₃, TaI₃, etc.), a chromium halide (for example, CrF₃, CrCl₃, CrBr₃, CrI₃, etc.), a molybdenum halide (for example, MoF₃, MoCl₃, MoBr₃, MoI₃, etc.), a tungsten halide (for example, WF₃, WCl₃, WBr₃, WI₃, etc.), a manganese halide (for example, MnF₂, MnCl₂, MnBr₂, MnI₂, etc.), a technetium halide (for example, TcF₂, TcCl₂, TcBr₂, TcI₂, etc.), a rhenium halide (for example, ReF₂, ReCl₂, ReBr₂, ReI₂, etc.), an iron halide (for example, FeF₂, FeCl₂, FeBr₂, FeI₂, etc.), a ruthenium halide (for example, RuF₂, RuCl₂, RuBr₂, RuI₂, etc.), an osmium halide (for example, OsF₂, OsCl₂, OsBr₂, OsI₂, etc.), a cobalt halide (for example, CoF₂, CoCl₂, CoBr₂, CoI₂, etc.), a rhodium halide (for example, RhF₂, RhCl₂, RhBr₂, RhI₂, etc.), an iridium halide (for example, IrF₂, IrCl₂, IrBr₂, IrI₂, etc.), a nickel halide (for example, NiF₂, NiCl₂, NiBr₂, NiI₂, etc.), a palladium halide (for example, PdF₂, PdCl₂, PdBr₂, PdI₂, etc.), a platinum halide (for example, PtF₂, PtCl₂, PtBr₂, PtI₂, etc.), a copper halide (for example, CuF, CuCl, CuBr, CuI, etc.), a silver halide (for example, AgF, AgCl, AgBr, AgI, etc.), a gold halide (for example, AuF, AuCl, AuBr, AuI, etc.), and the like.

Examples of a post-transition metal halide may include a zinc halide (for example, ZnF₂, ZnCl₂, ZnBr₂, ZnI₂, etc.), an indium halide (for example, InI₃, etc.), a tin halide (for example, SnI₂, etc.), and the like.

Examples of a lanthanide metal halide may include YbF, YbF₂, YbF₃, SmF₃, YbCl, YbCl₂, YbCl₃, SmCl₃, YbBr, YbBr₂, YbBr₃, SmBr₃, YbI, YbI₂, YbI₃, SmI₃, and the like.

Examples of a metalloid halide may include antimony halide (for example, SbCl₅, etc.) and the like.

Examples of a metal telluride may include an alkali metal telluride (for example, Li₂Te, Na₂Te, K₂Te, Rb₂Te, Cs₂Te, etc.), an alkaline earth metal telluride (for example, BeTe, MgTe, CaTe, SrTe, BaTe, etc.), a transition metal telluride (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, CuzTe, CuTe, Ag₂Te, AgTe, Au₂Te, etc.), a post-transition metal telluride (for example, ZnTe, etc.), a lanthanide metal telluride (for example, LaTe, CeTe, PrTe, NdTe, PmTe, EuTe, GdTe, TbTe, DyTe, HoTe, ErTe, TmTe, YbTe, LuTe, etc.), and the like.

### [Emission Layer in Interlayer 130]

When the light-emitting device 10 is a full-color light-emitting device, the emission layer may be patterned into a red emission layer, a green emission layer, and/or a blue emission layer, according to a subpixel. In an embodiment, the emission layer may have a stacked structure of two or more layers of a red emission layer, a green emission layer, and a blue emission layer, in which the two or more layers may contact each other or may be separated from each other, to emit white light. In embodiments, the emission layer may include two or more materials of a red light-emitting material, a green light-emitting material, and a blue light-emitting material, in which the two or more materials may be mixed with each other in a single layer to emit white light.

In an embodiment, the emission layer may include a host and a dopant (or an emitter). In an embodiment, the emission layer may further include an auxiliary dopant that promotes energy transfer to a dopant (or to an emitter), in addition to the host and the dopant (or the emitter). When the emission layer includes the dopant (or the emitter) and the auxiliary dopant, the dopant (or the emitter) and the auxiliary dopant may be different from each other.

An amount of the dopant (or the emitter) in the emission layer may be in a range of about 0.01 parts by weight to about 15 parts by weight, based on 100 parts by weight of the host.

In embodiments, the emission layer may include a quantum dot.

In embodiments, the emission layer may include a delayed fluorescence material. The delayed fluorescence material may serve as a host or as a dopant in the emission layer.

The thickness of the emission layer may be in a range of about 100 Å to about 1,000 Å. For example, the thickness of the emission layer may be in a range of about 200 Å to about 600 Å. When the thickness of the emission layer is within any of these ranges, excellent luminescence characteristics may be obtained without a substantial increase in driving voltage.

### [Host]

In an embodiment, the host may include a compound represented by Formula 301:

[Ar₃₀₁]_{xb11}-[(L₃₀₁)_{xb1}-R₃₀₁]_{xb21} [Formula 301]

### In Formula 301,

Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xb11 may be 1, 2, or 3,
xb1 may be an integer from 0 to 5,
R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),
xb21 may be an integer from 1 to 5, and
Q₃₀₁ to Q₃₀₃ are each independently be the same as described in connection with Q₁.

In an embodiment, in Formula 301, when xb11 is 2 or more, two or more of Ar₃₀₁ may be linked to each other via a single bond.

In embodiments, the host may include a compound represented by Formula 301-1, a compound represented by Formula 301-2, or any combination thereof:

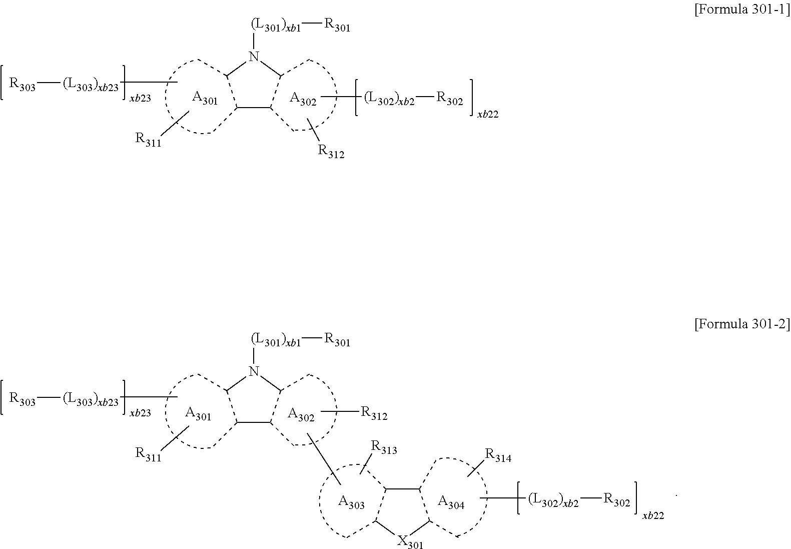

In Formulae 301-1 and 301-2,
ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
X₃₀₁ may be O, S, N-[(L₃₀₄)_{xb4}-R₃₀₄], C(R₃₀₄)(R₃₀₅), or Si(R₃₀₄)(R₃₀₅),
xb22 and xb23 may each independently be 0, 1, or 2,
L₃₀₁, xb1, and R₃₀₁ are each the same as described herein,
L₃₀₂ to L₃₀₄ may each independently be the same as described in connection with L₃₀₁,
xb2 to xb4 may each independently be the same as described in connection with xb1, and
R₃₀₂ to R₃₀₅ and R₃₁₁ to R₃₁₄ may each independently be the same as described in connection with R₃₀₁.

In embodiments, the host may include an alkali earth metal complex, a post-transition metal complex, or any combination thereof. In embodiments, the host may include a Be complex (for example, Compound H55), an Mg complex, a Zn complex, or any combination thereof.

In embodiments, the host may include: one of Compounds H1 to H128; 9,10-di(2-naphthyl)anthracene (ADN); 2-methyl-9,10-bis(naphthalen-2-yl)anthracene (MADN); 9,10-di(2-naphthyl)-2-t-butyl-anthracene (TBADN); 4,4′-bis(N-carbazolyl)-1,1′-biphenyl (CBP); 1,3-di-9-carbazolylbenzene (mCP); 1,3,5-tri(carbazol-9-yl)benzene (TCP); or any combination thereof:

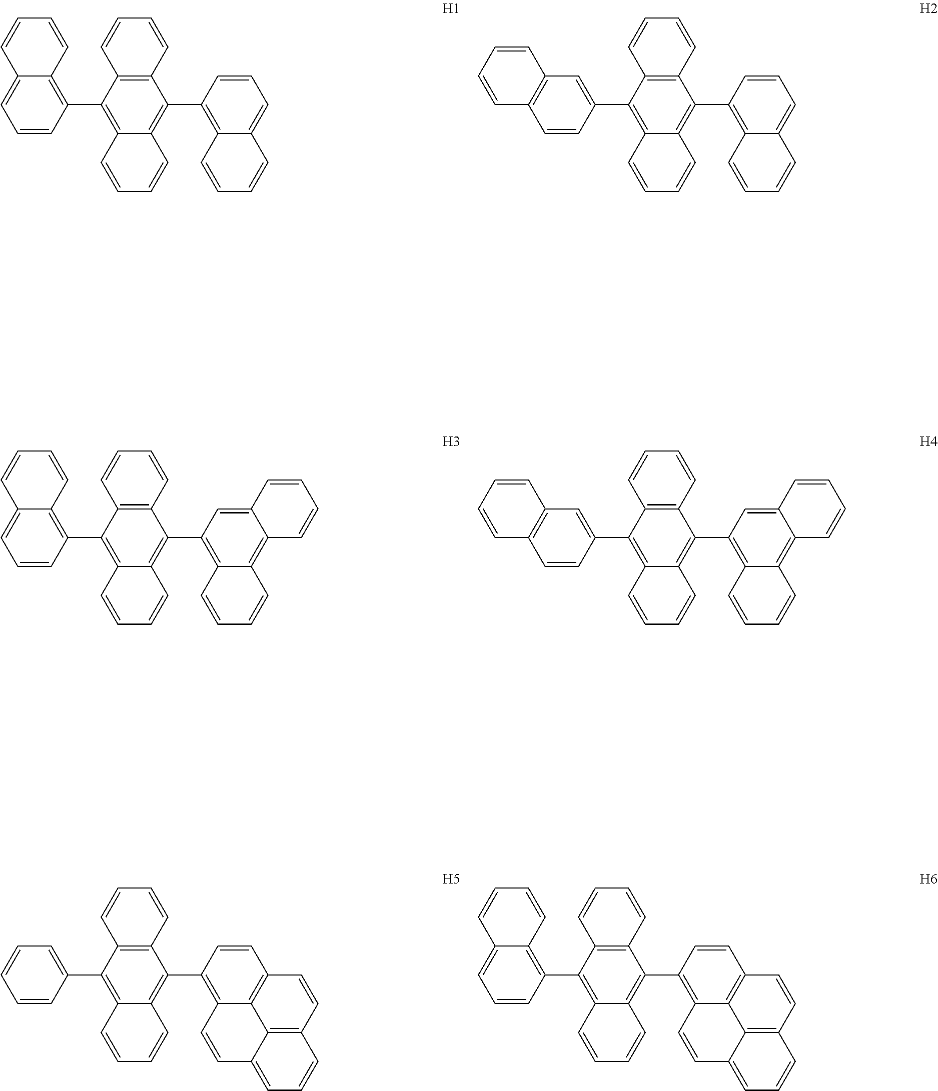

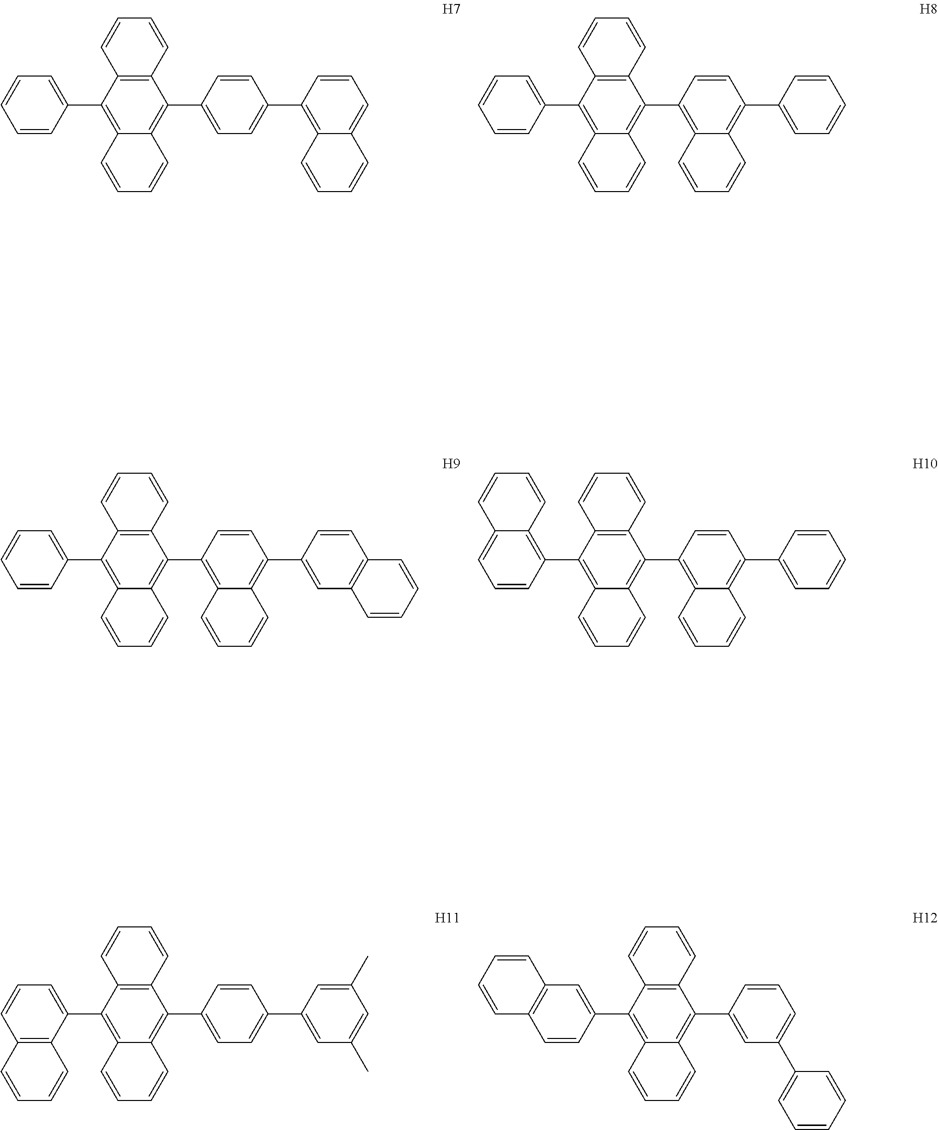

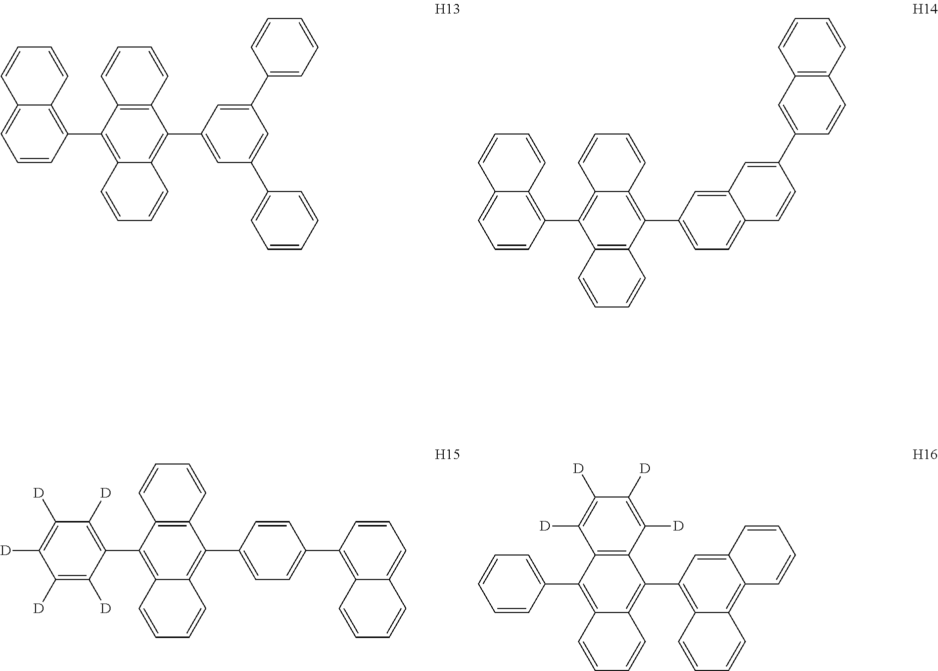

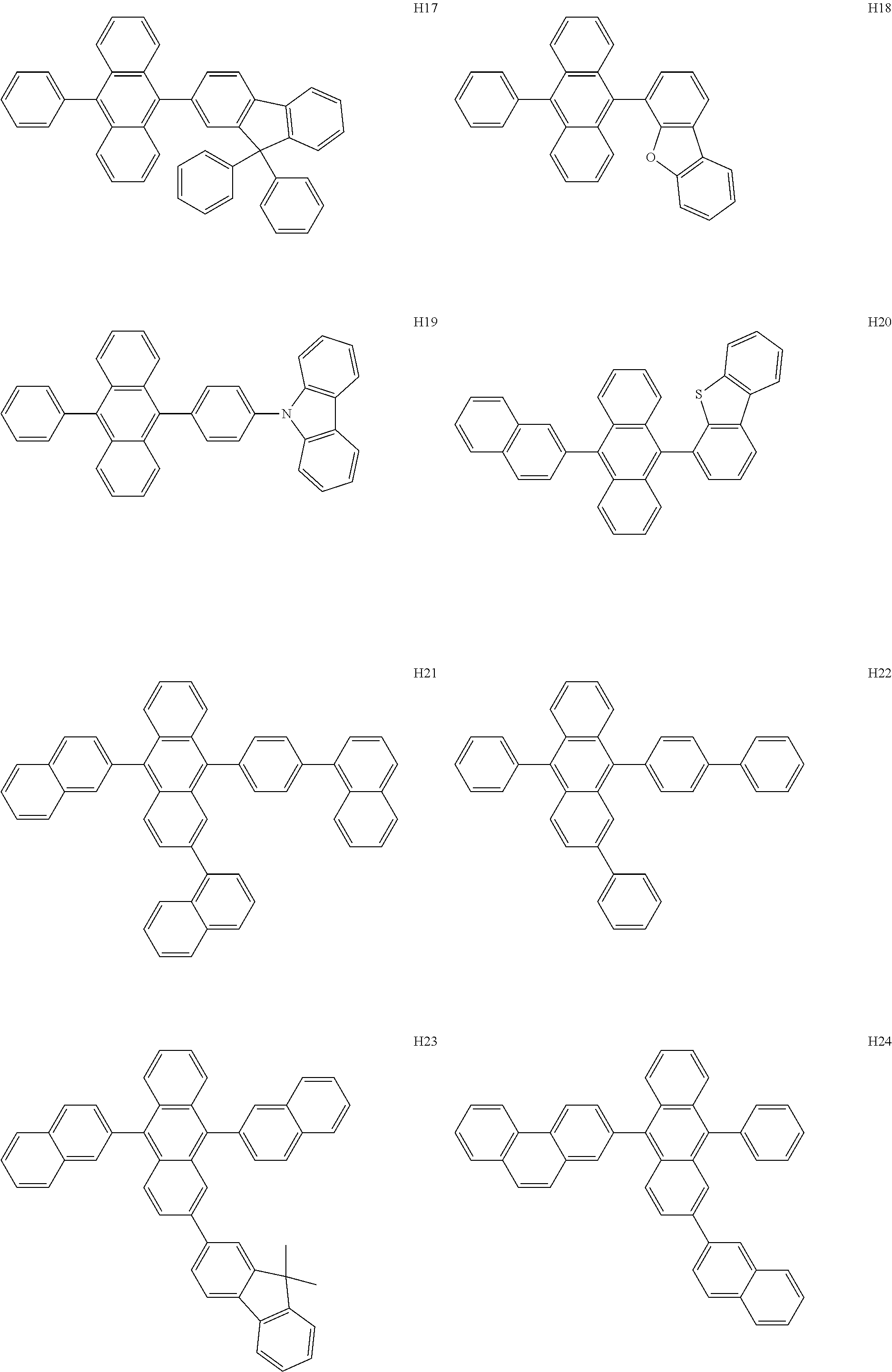

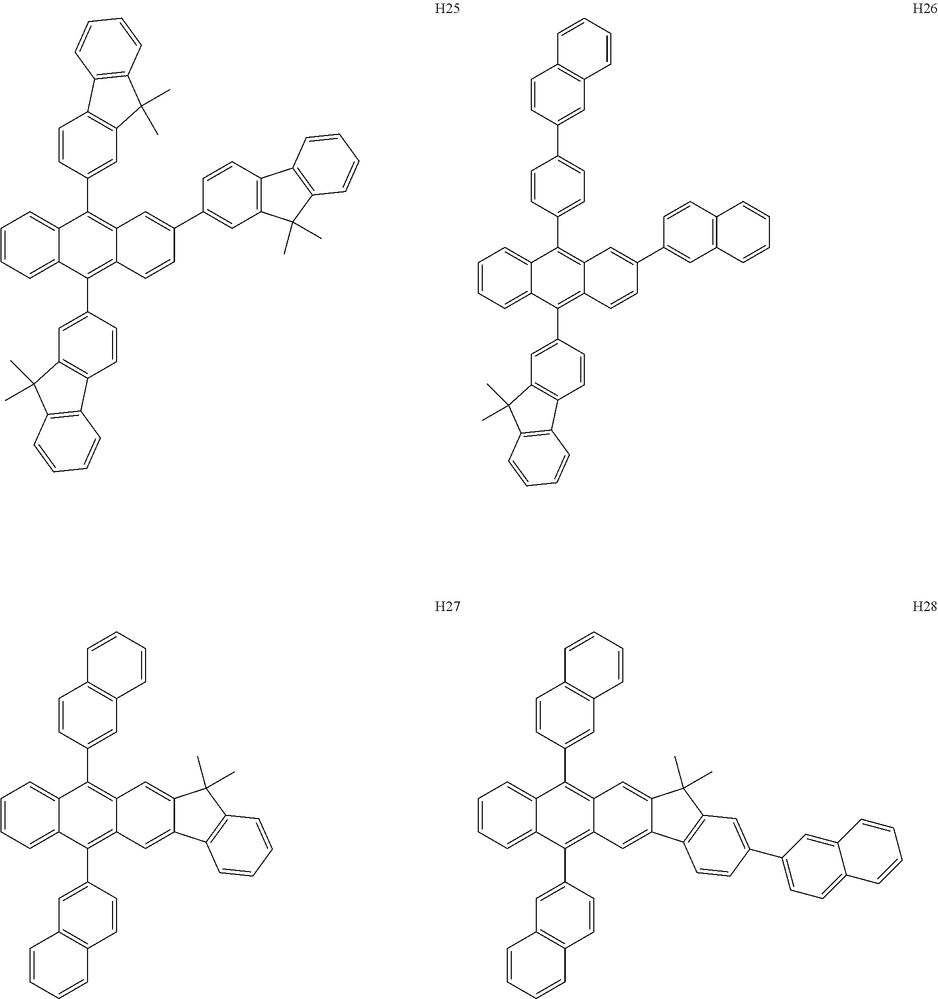

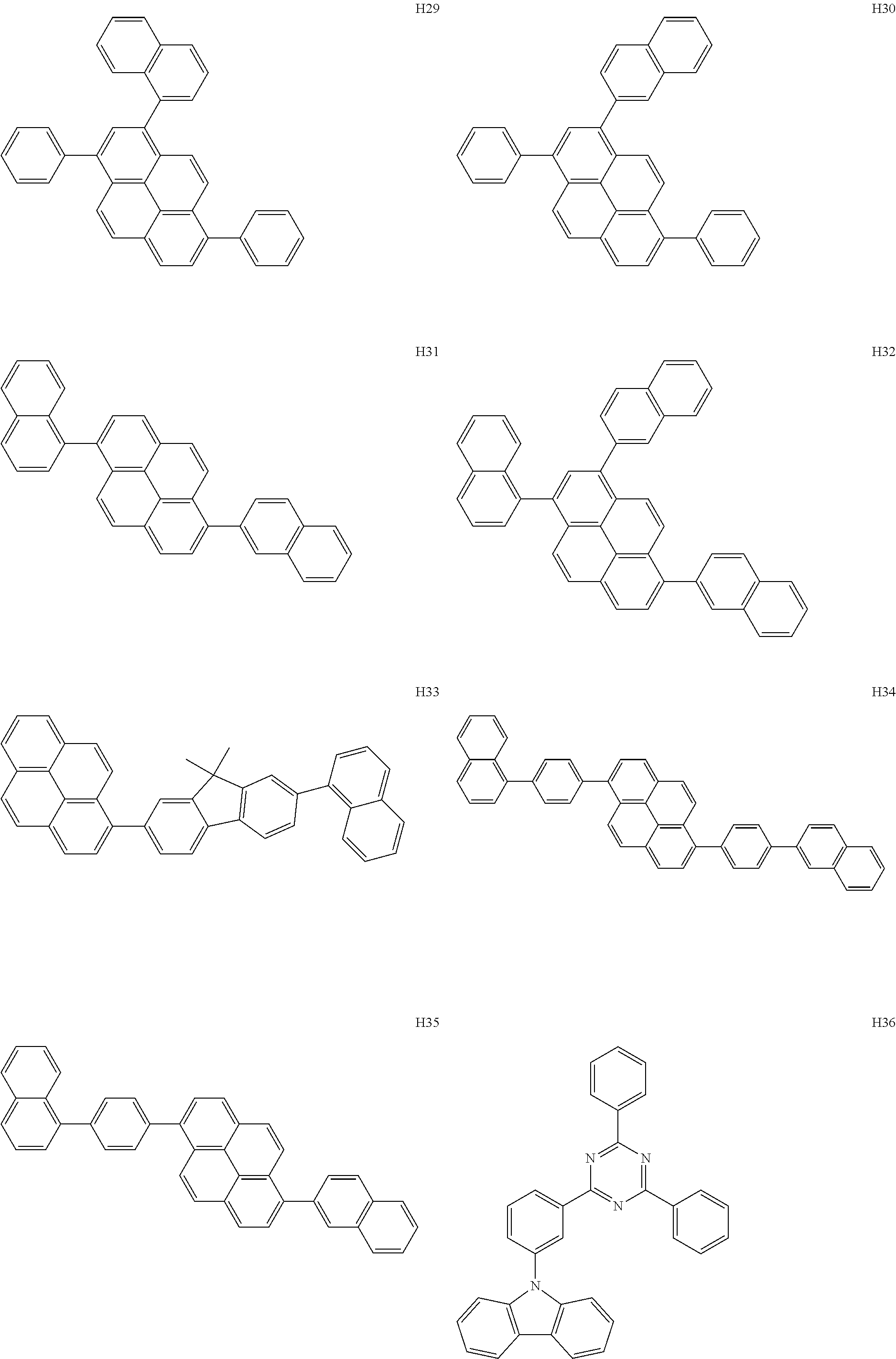

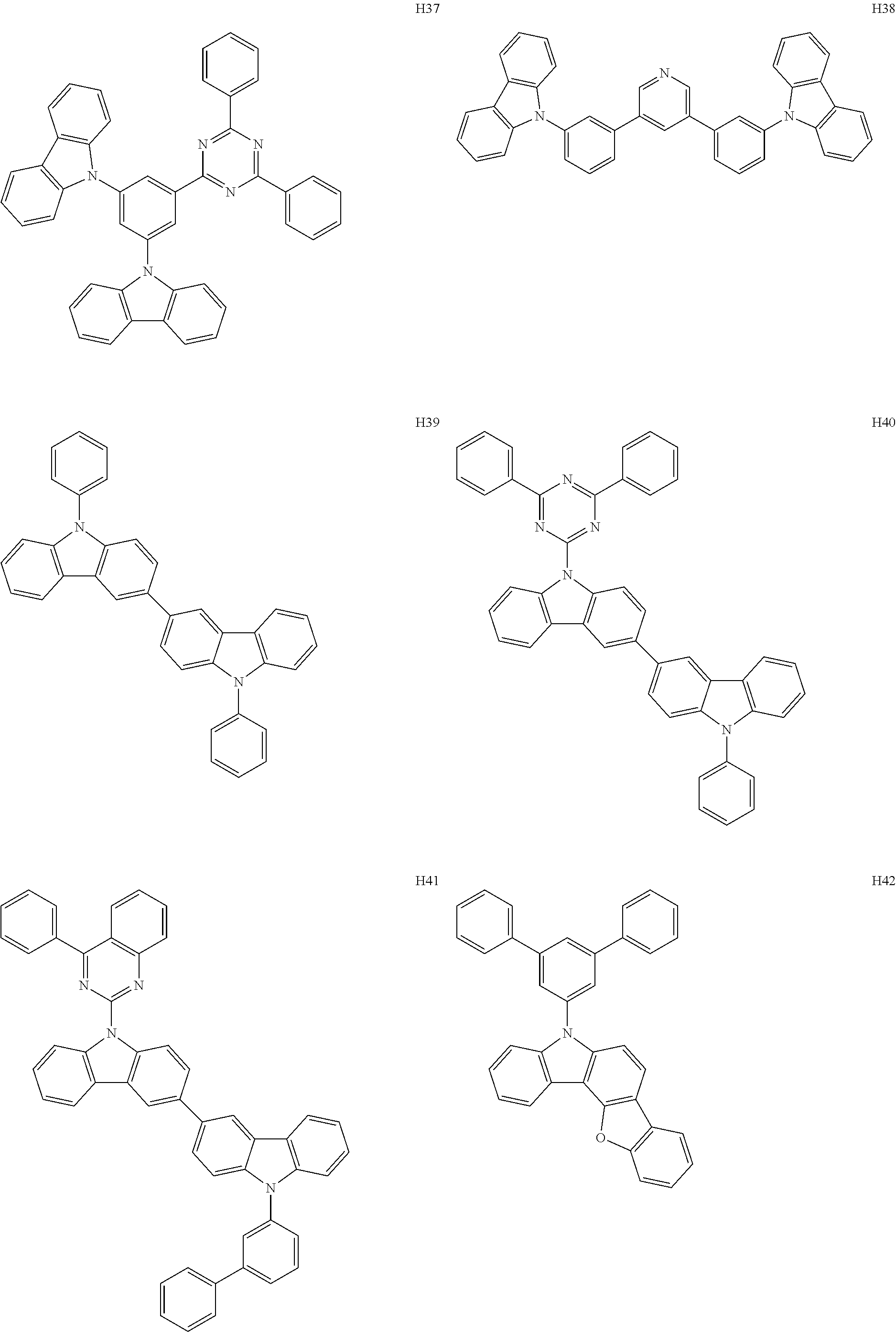 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-n2c3ccccc3c3cc(-c4ccc5c(c4)c4ccccc4n5-c4ccccc4)ccc32)cc1 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2cccc(-n3c4ccccc4c4cc(-c5ccc6c(c5)c5ccccc5n6-c5nc(-c6ccccc6)c6ccccc6n5)ccc43)c2)cc1 ; c1ccc(-c2cc(-c3ccccc3)cc(-n3c4ccccc4c4c5oc6ccccc6c5ccc43)c2)cc1]

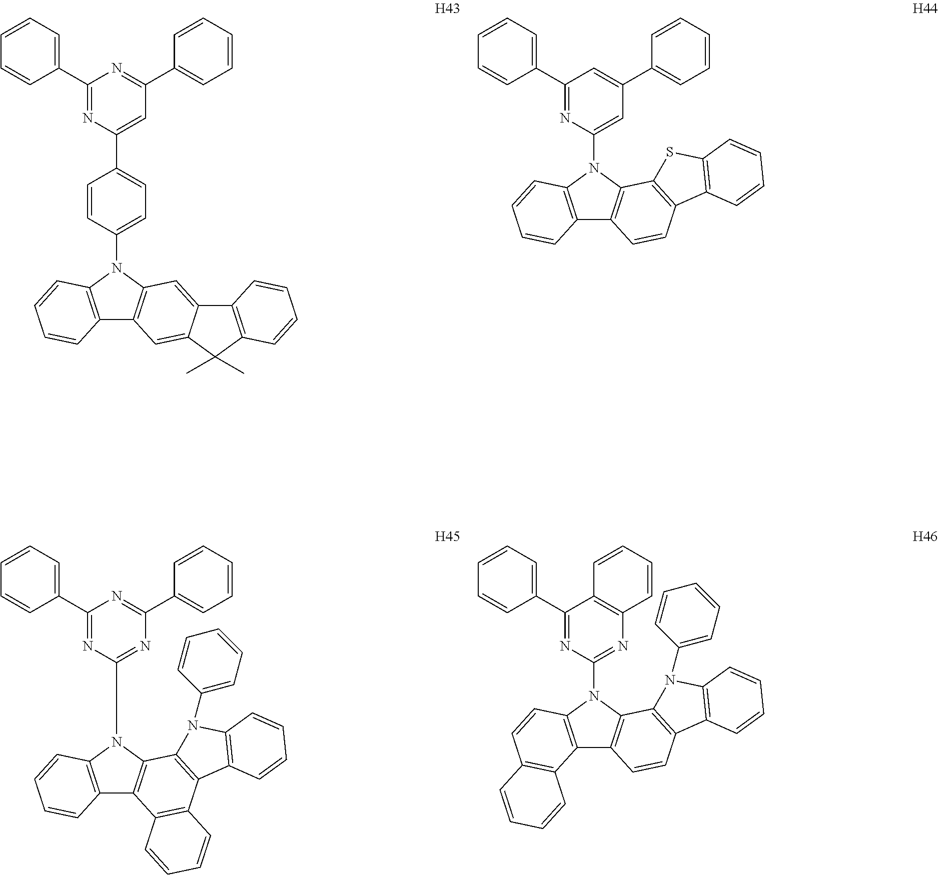 [SMILES: CC1(C)c2ccccc2-c2cc3c(cc21)c1ccccc1n3-c1ccc(-c2cc(-c3ccccc3)nc(-c3ccccc3)n2)cc1 ; c1ccc(-c2cc(-c3ccccc3)nc(-n3c4ccccc4c4ccc5c6ccccc6sc5c43)c2)cc1 ; c1ccc(-c2nc(-c3ccccc3)nc(-n3c4ccccc4c4c5ccccc5c5c6ccccc6n(-c6ccccc6)c5c43)n2)cc1 ; c1ccc(-c2nc(-n3c4ccc5ccccc5c4c4ccc5c6ccccc6n(-c6ccccc6)c5c43)nc3ccccc23)cc1]

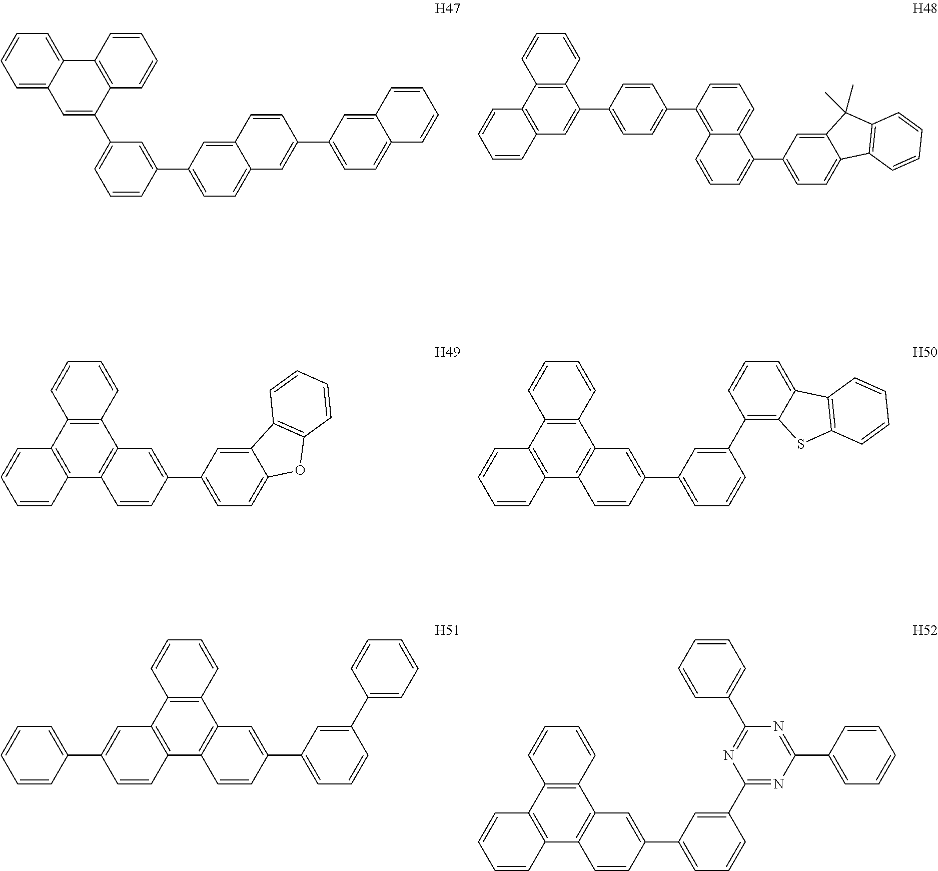

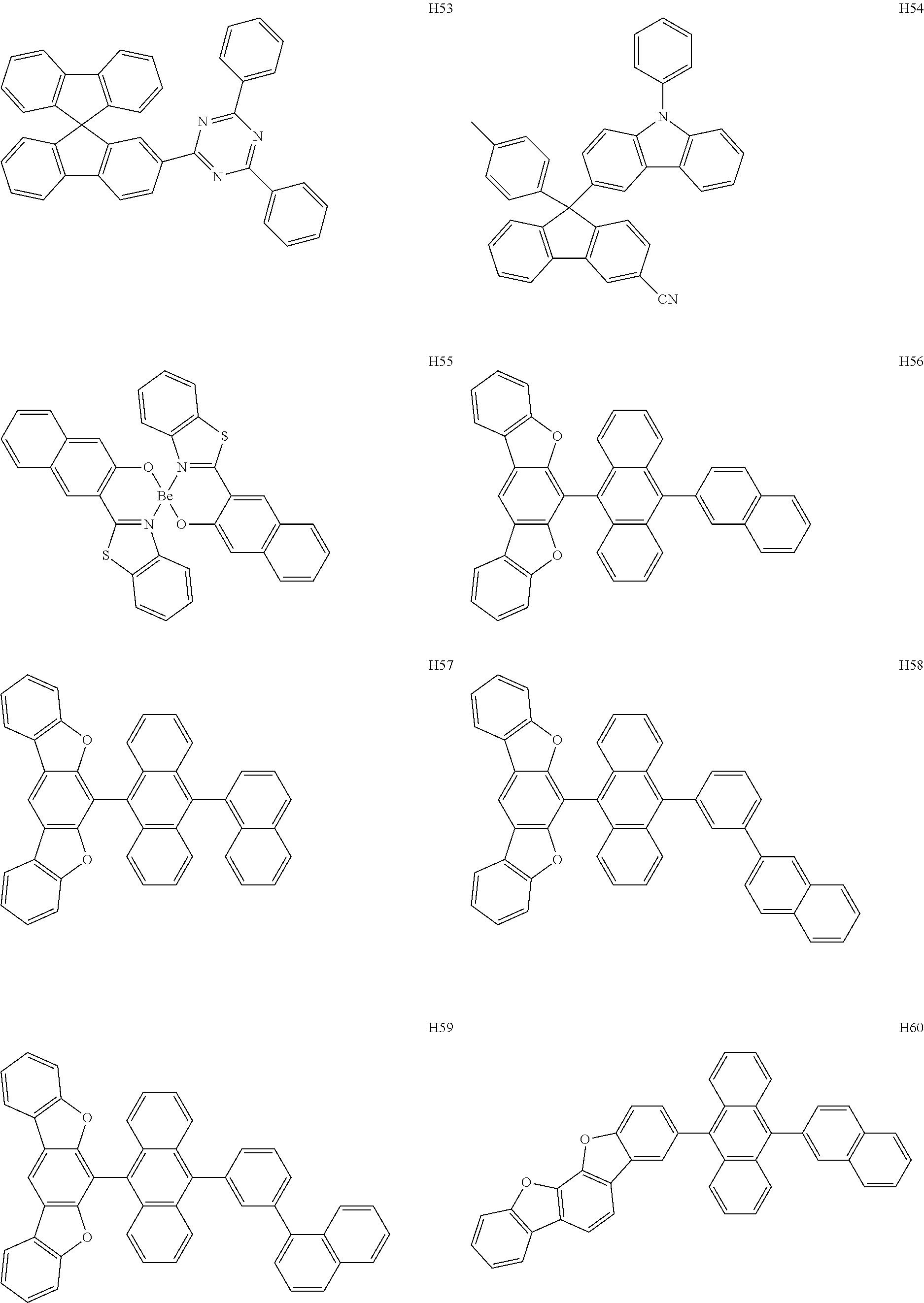

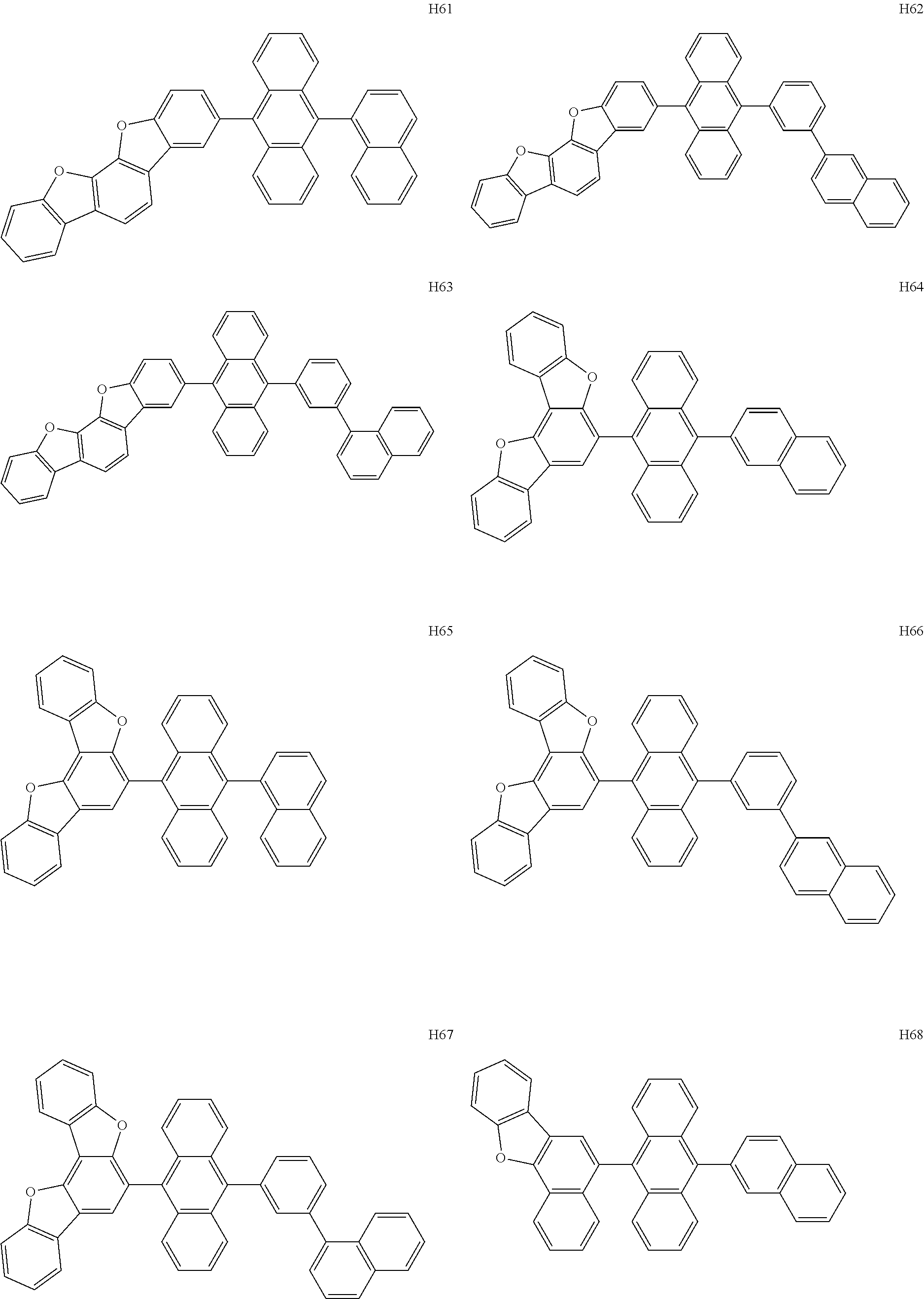

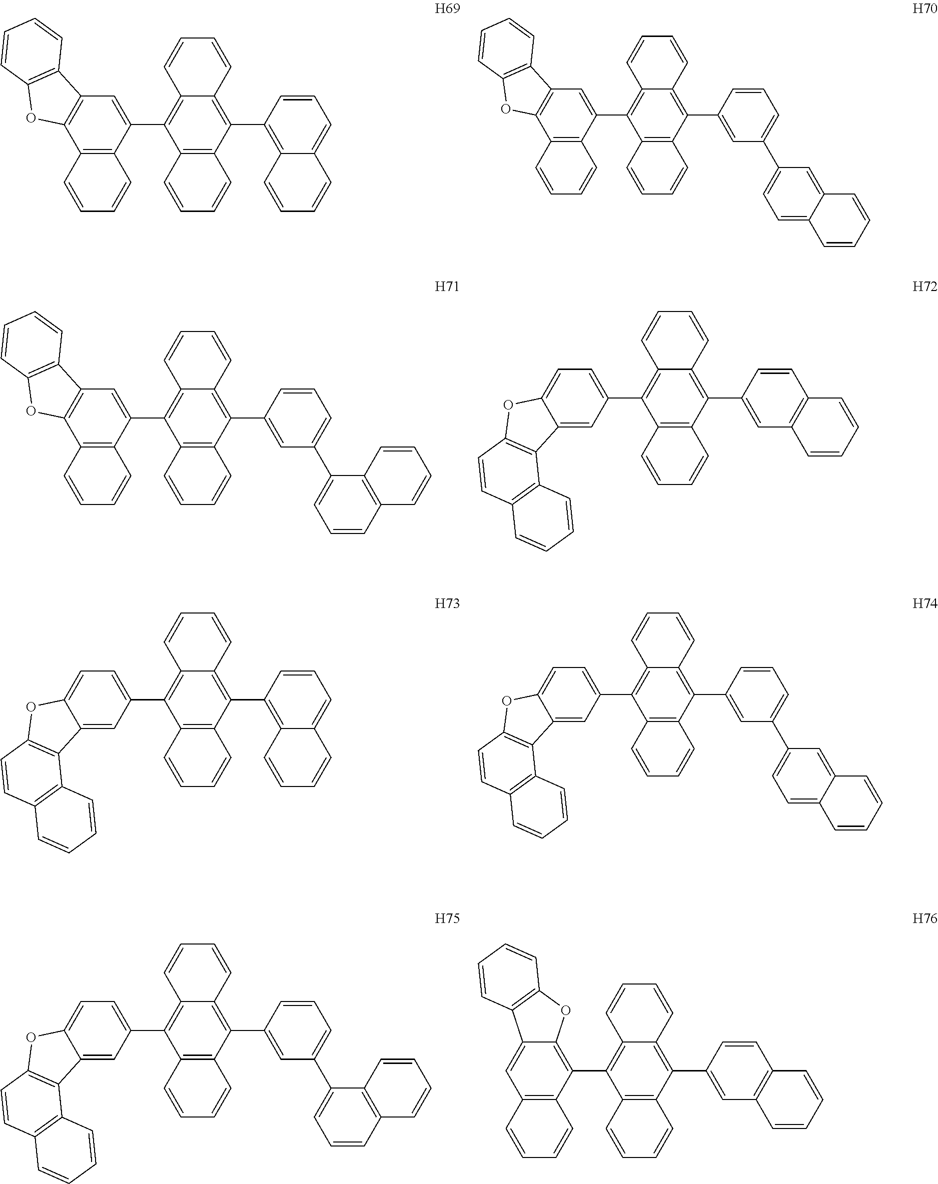

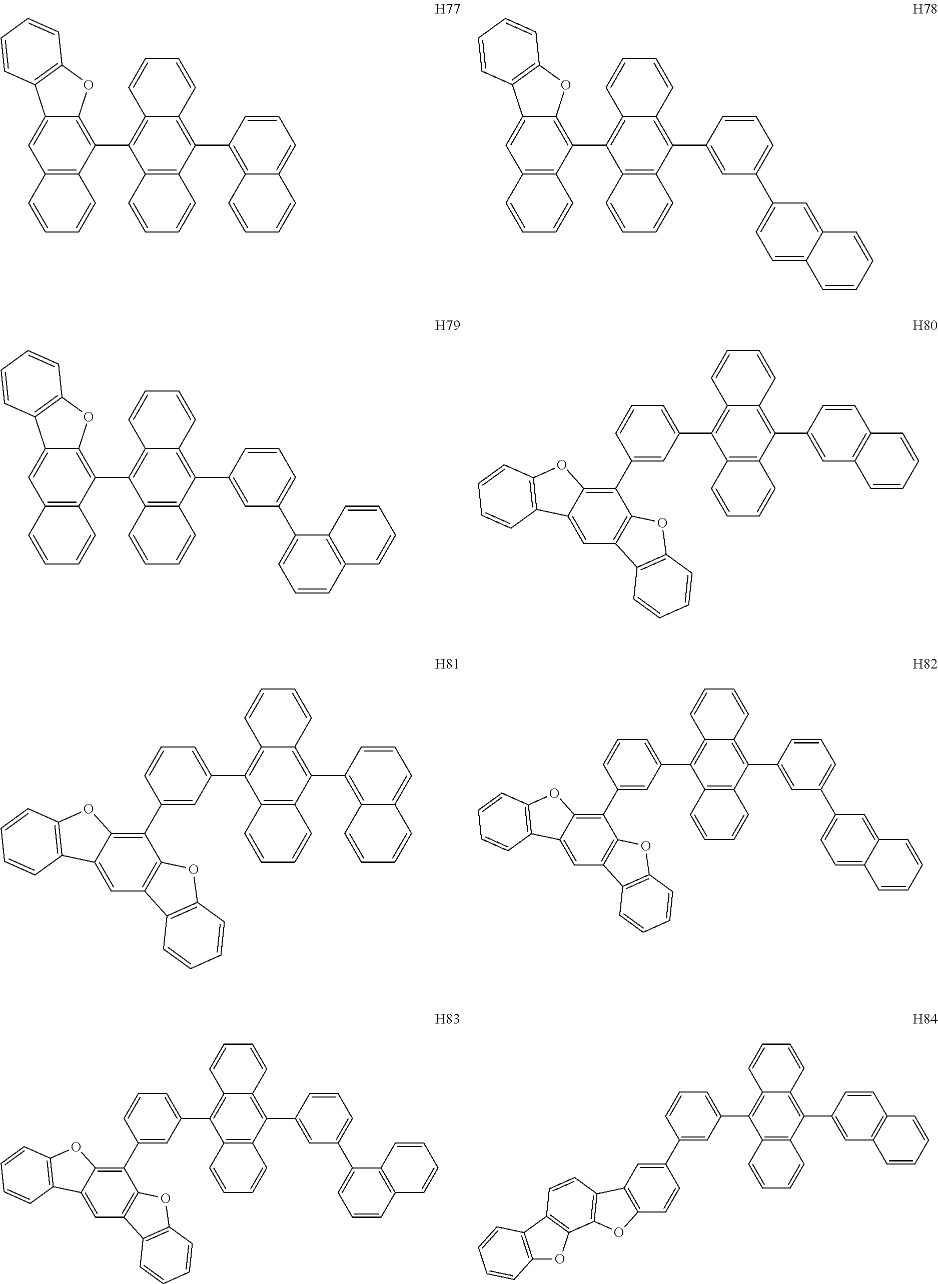

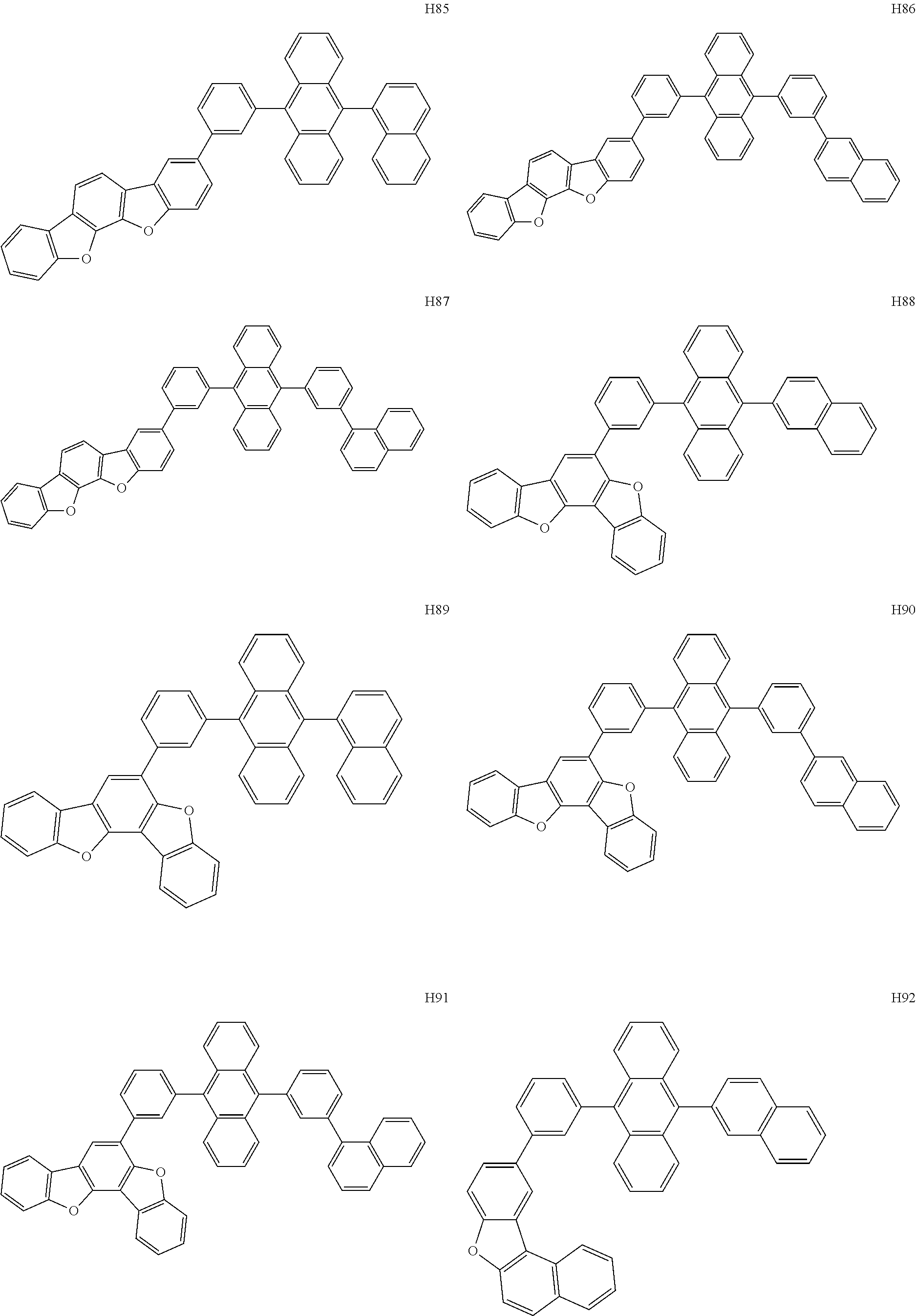

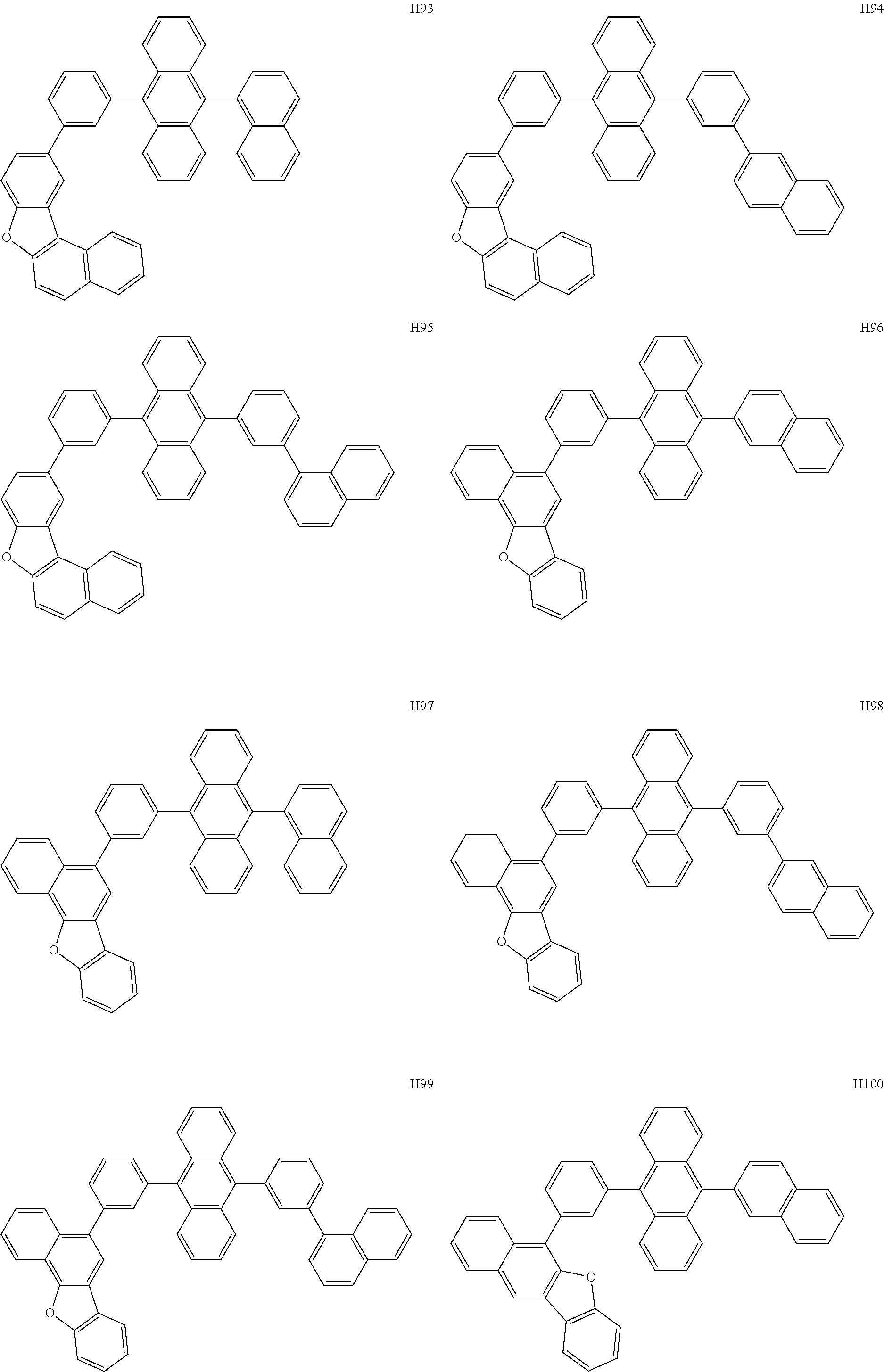

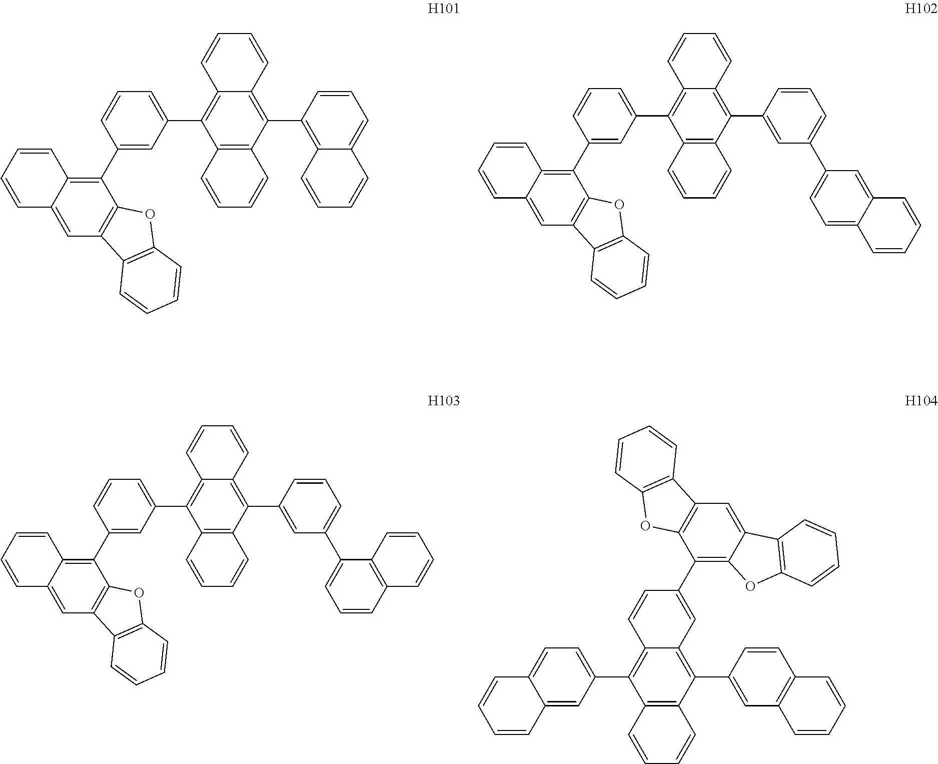 [SMILES: c1cc(-c2c3ccccc3c(-c3cccc4ccccc34)c3ccccc23)cc(-c2c3ccccc3cc3c2oc2ccccc23)c1 ; c1cc(-c2ccc3ccccc3c2)cc(-c2c3ccccc3c(-c3cccc(-c4c5ccccc5cc5c4oc4ccccc45)c3)c3ccccc23)c1 ; c1cc(-c2cccc3ccccc23)cc(-c2c3ccccc3c(-c3cccc(-c4c5ccccc5cc5c4oc4ccccc45)c3)c3ccccc23)c1 ; c1ccc2cc(-c3c4ccccc4c(-c4ccc5ccccc5c4)c4cc(-c5c6oc7ccccc7c6cc6c5oc5ccccc56)ccc34)ccc2c1]

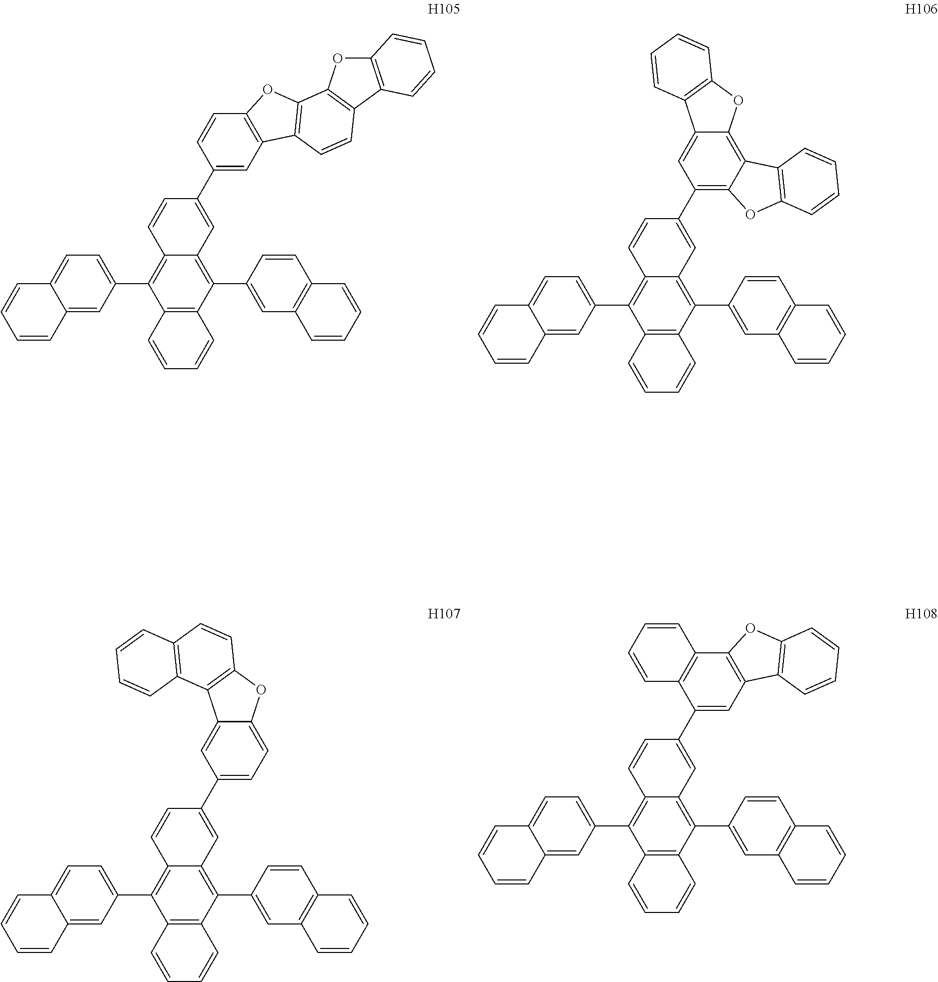 [SMILES: c1ccc2cc(-c3c4ccccc4c(-c4ccc5ccccc5c4)c4cc(-c5ccc6oc7c(ccc8c9ccccc9oc87)c6c5)ccc34)ccc2c1 ; c1ccc2cc(-c3c4ccccc4c(-c4ccc5ccccc5c4)c4cc(-c5cc6c7ccccc7oc6c6c5oc5ccccc56)ccc34)ccc2c1 ; c1ccc2cc(-c3c4ccccc4c(-c4ccc5ccccc5c4)c4cc(-c5ccc6oc7ccc8ccccc8c7c6c5)ccc34)ccc2c1 ; c1ccc2cc(-c3c4ccccc4c(-c4ccc5ccccc5c4)c4cc(-c5cc6c7ccccc7oc6c6ccccc56)ccc34)ccc2c1]

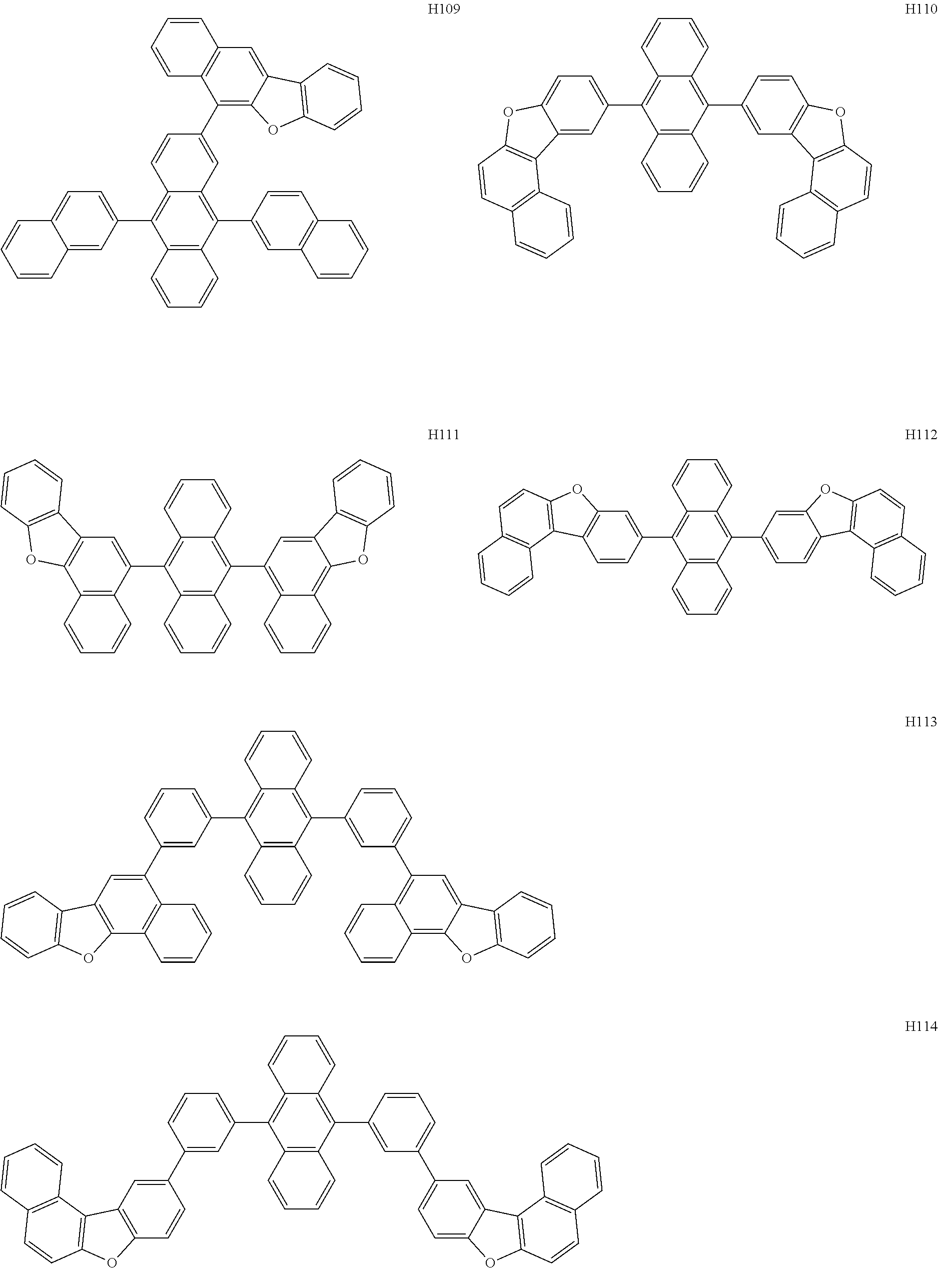

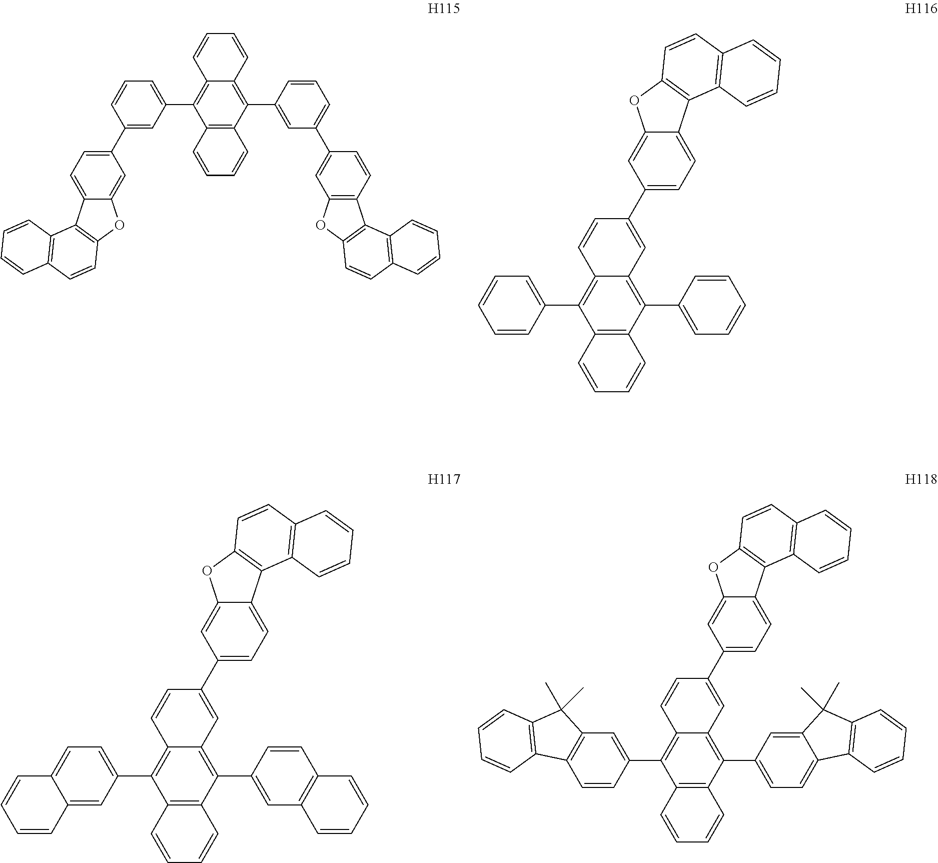 [SMILES: c1cc(-c2ccc3c(c2)oc2ccc4ccccc4c23)cc(-c2c3ccccc3c(-c3cccc(-c4ccc5c(c4)oc4ccc6ccccc6c45)c3)c3ccccc23)c1 ; c1ccc(-c2c3ccccc3c(-c3ccccc3)c3cc(-c4ccc5c(c4)oc4ccc6ccccc6c45)ccc23)cc1 ; c1ccc2cc(-c3c4ccccc4c(-c4ccc5ccccc5c4)c4cc(-c5ccc6c(c5)oc5ccc7ccccc7c56)ccc34)ccc2c1 ; CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21]

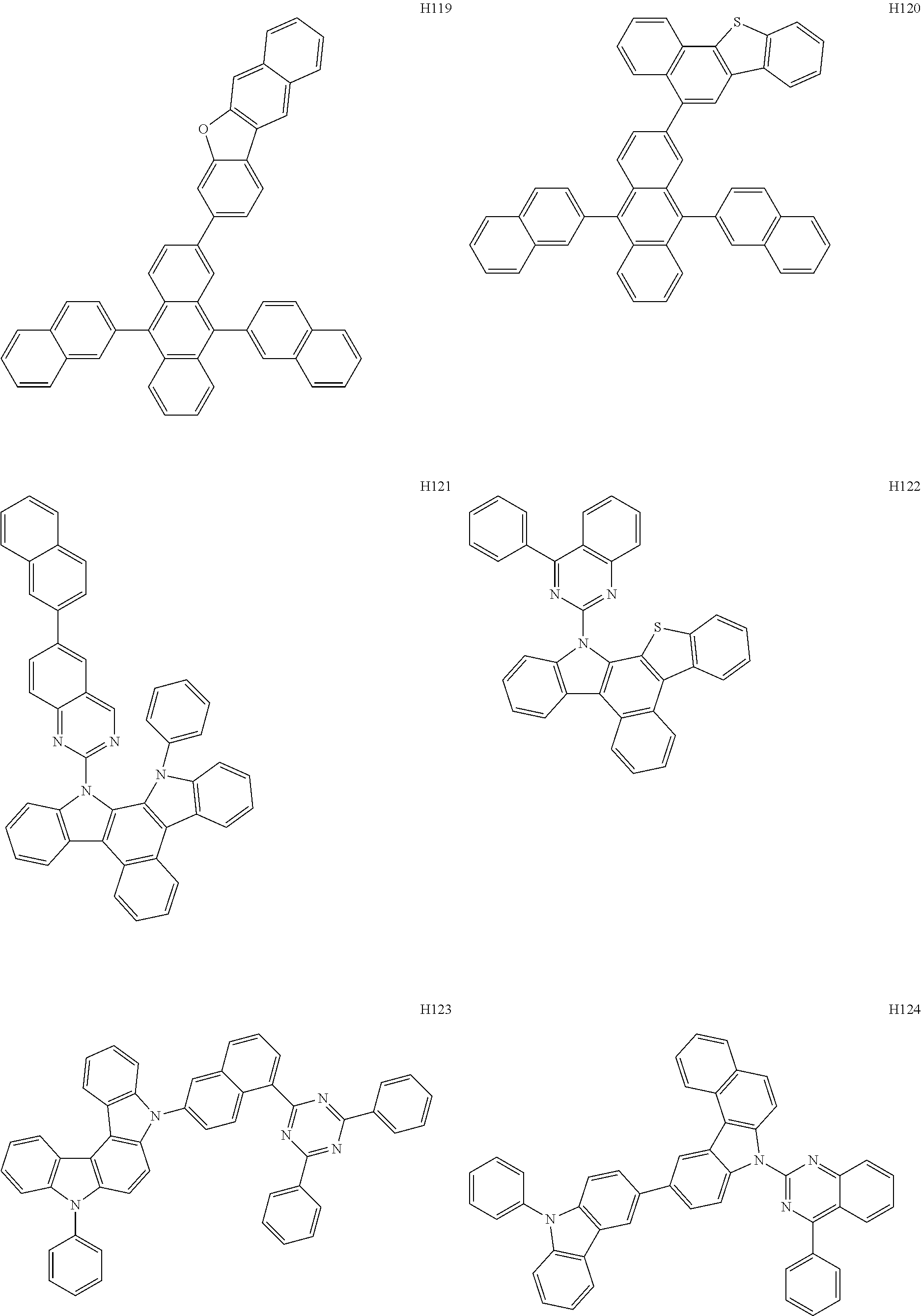

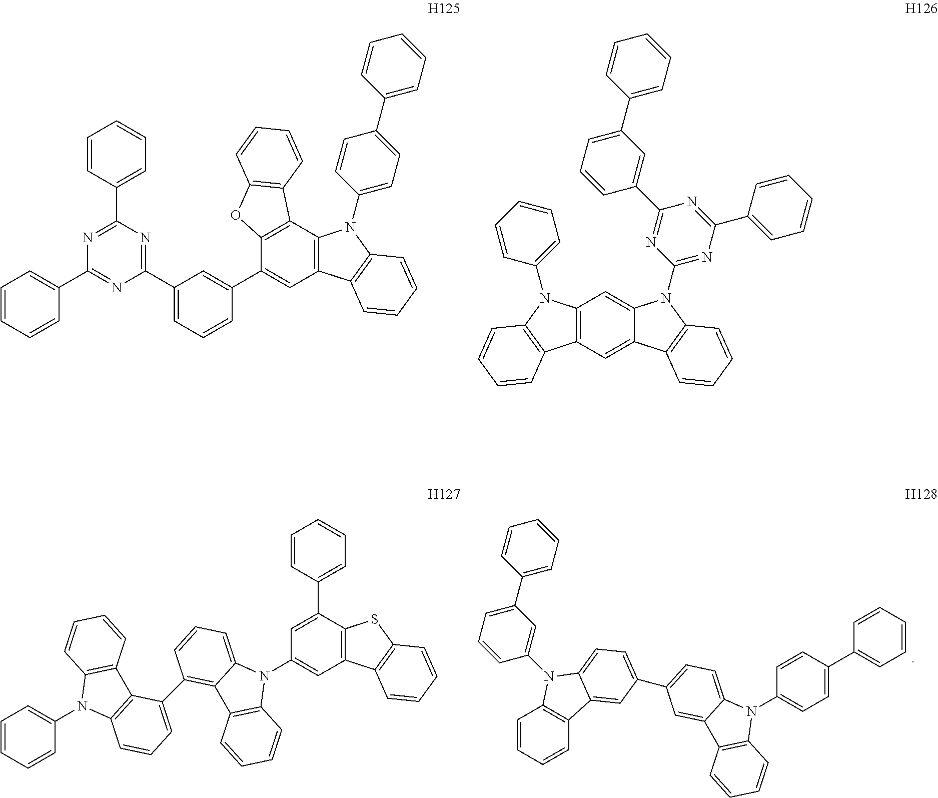 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-c2cccc(-c3nc(-c4ccccc4)nc(-n4c5ccccc5c5cc6c7ccccc7n(-c7ccccc7)c6cc54)n3)c2)cc1 ; c1ccc(-c2cc(-n3c4ccccc4c4c(-c5cccc6c5c5ccccc5n6-c5ccccc5)cccc43)cc3c2sc2ccccc23)cc1 ; c1ccc(-c2ccc(-n3c4ccccc4c4cc(-c5ccc6c(c5)c5ccccc5n6-c5cccc(-c6ccccc6)c5)ccc43)cc2)cc1]

In embodiments, the host may include a silicon-containing compound, a phosphine oxide-containing compound, or any combination thereof.

The host may have various modifications. For example, the host may include only one kind of compound, or may include two or more different kinds of compounds.

### [Phosphorescent Dopant]

The emission layer may include a phosphorescent dopant.

The phosphorescent dopant may include at least one transition metal as a central metal. Accordingly, the phosphorescent dopant may correspond to the fifth compound.

The phosphorescent dopant may include a monodentate ligand, a bidentate ligand, a tridentate ligand, a tetradentate ligand, a pentadentate ligand, a hexadentate ligand, or any combination thereof.

The phosphorescent dopant may be electrically neutral.

In an embodiment, the phosphorescent dopant may include an organometallic compound represented by Formula 401:

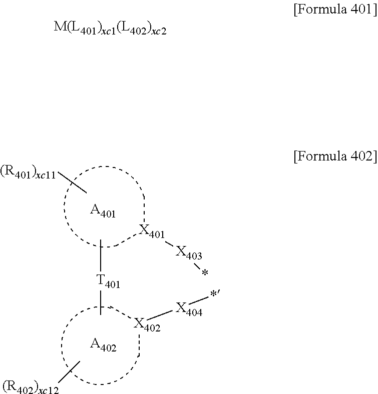

In Formulae 401 and 402,
M may be a transition metal (for example, iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),
L₄₀₁ may be a ligand represented by Formula 402, and xc1 may be 1, 2, or 3, wherein when xc1 is 2 or more, two or more of L₄₀₁ may be identical to or different from each other,
L₄₀₂ may be an organic ligand, and xc2 may be 0, 1, 2, 3, or 4, wherein when xc2 is 2 or more, two or more of L₄₀₂ may be identical to or different from each other,
X₄₀₁ and X₄₀₂ may each independently be N or C,
ring A₄₀₁ and ring A₄₀₂ may each independently be a C₃-C₆₀ carbocyclic group or a C₁-C₆₀ heterocyclic group,
T₄₀₁ may be a single bond, *—O—*′, *—S—*′, *—C(═O)—*′, *—N(Q₄₁₁)—*′, *—C(Q₄₁₁)(Q₄₁₂)-*′, *—C(Q₄₁₁)═C(Q₄₁₂)-*′, *—C(Q₄₁₁)═*′, or *═C═*′,
X₄₀₃ and X₄₀₄ may each independently be a chemical bond (for example, a covalent bond or a coordination bond), O, S, N(Q₄₁₃), B(Q₄₁₃), P(Q₄₁₃), C(Q₄₁₃)(Q₄₁₄), or Si(Q₄₁₃)(Q₄₁₄),
Q₄₁₁ to Q₄₁₄ may each independently be the same as described in connection with Q₁,
R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₅-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),
Q₄₀₁ to Q₄₀₃ may each independently be the same as described in connection with Q₁,
xc11 and xc12 may each independently be an integer from 0 to 10, and
* and *′ in Formula 402 each indicate a binding site to M in Formula 401.

In an embodiment, in Formula 402, X₄₀₁ may be nitrogen and X₄₀₂ may be carbon, or X₄₀₁ and X₄₀₂ may each be nitrogen.

In an embodiment, in Formula 401, when xc1 is 2 or more, two ring A₄₀₁(s) among two or more of L₄₀₁ may optionally be linked to each other via T₄₀₂, which may be a linking group, and two ring A₄₀₂(s) among two or more of L₄₀₁ may optionally be linked to each other via T₄₀₃, which may be a linking group (see Compounds PD1 to PD4 and PD7). T₄₀₂ and T₄₀₃ may each independently be the same as described in connection with T₄₀₁.

In Formula 401, L₄₀₂ may be an organic ligand. For example, L₄₀₂ may include a halogen group, a diketone group (for example, an acetylacetonate group), a carboxylic acid group (for example, a picolinate group), —C(═O), an isonitrile group, a —CN group, a phosphorus group (for example, a phosphine group, a phosphite group, etc.), or any combination thereof.

The phosphorescent dopant may include, for example, one of Compounds PD1 to PD39, or any combination thereof:

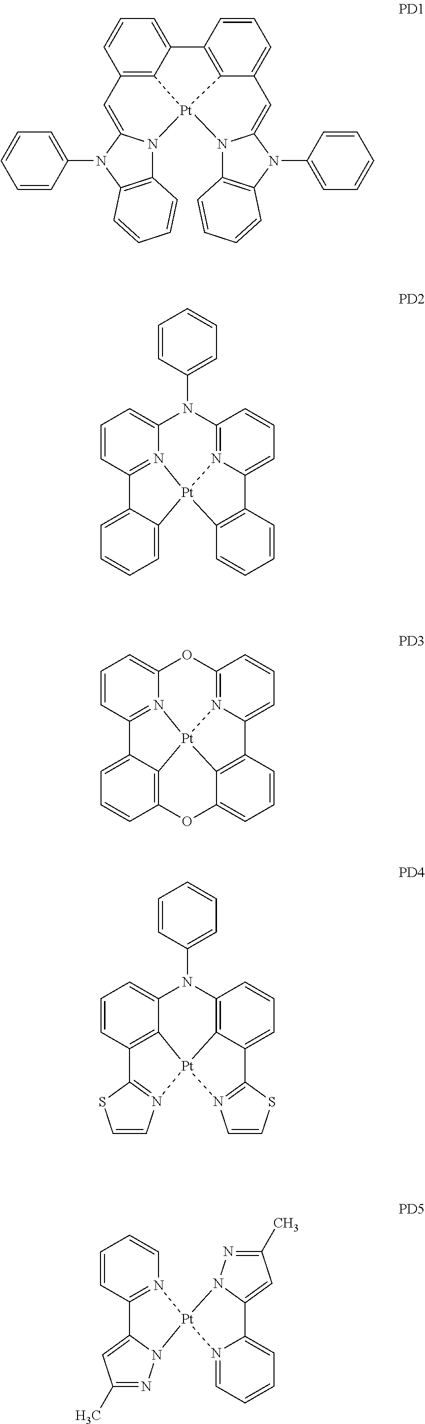

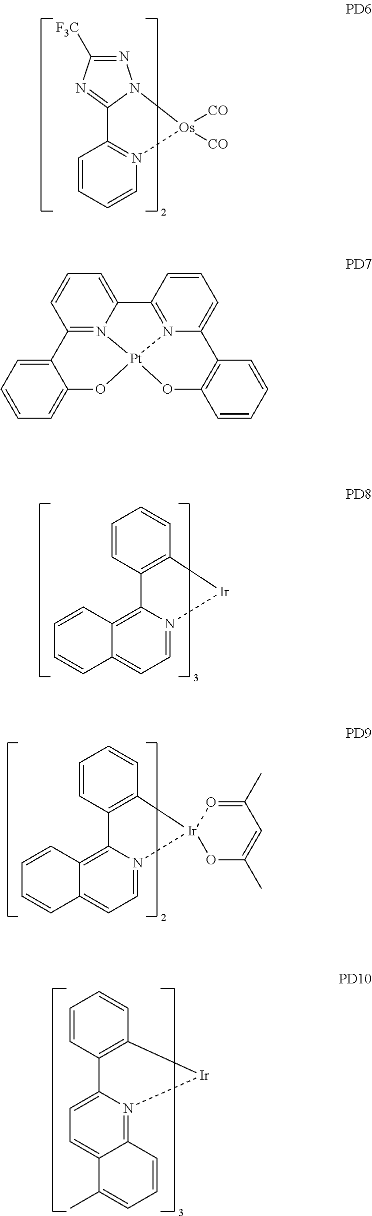

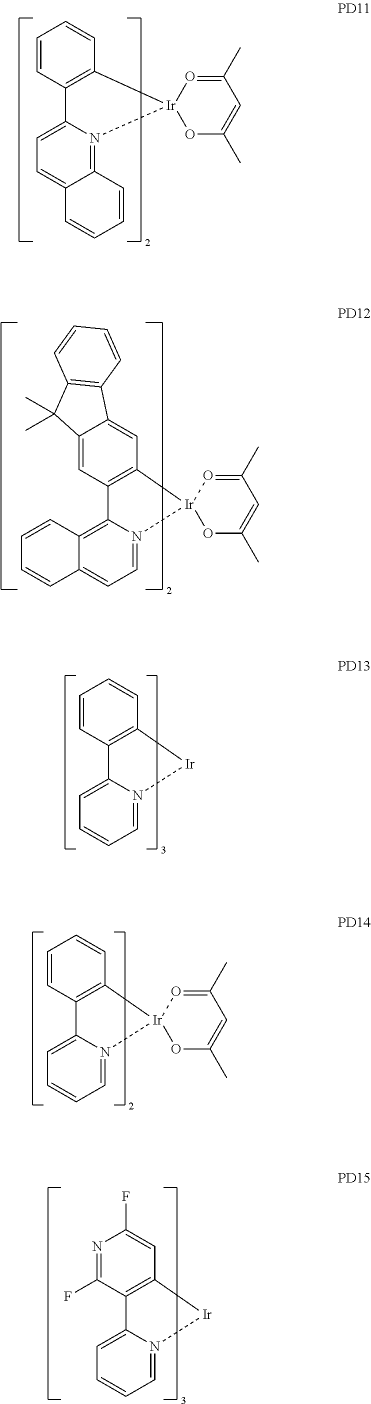

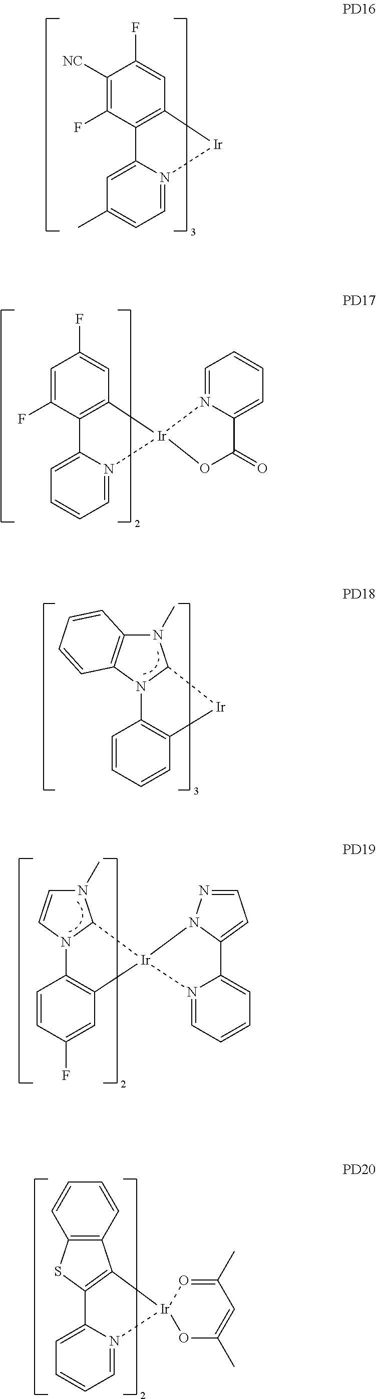

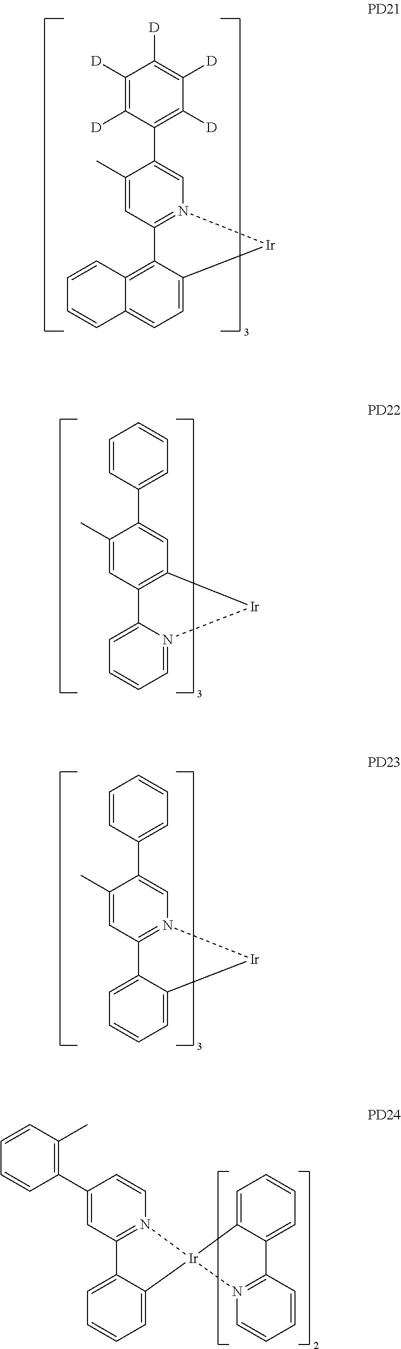

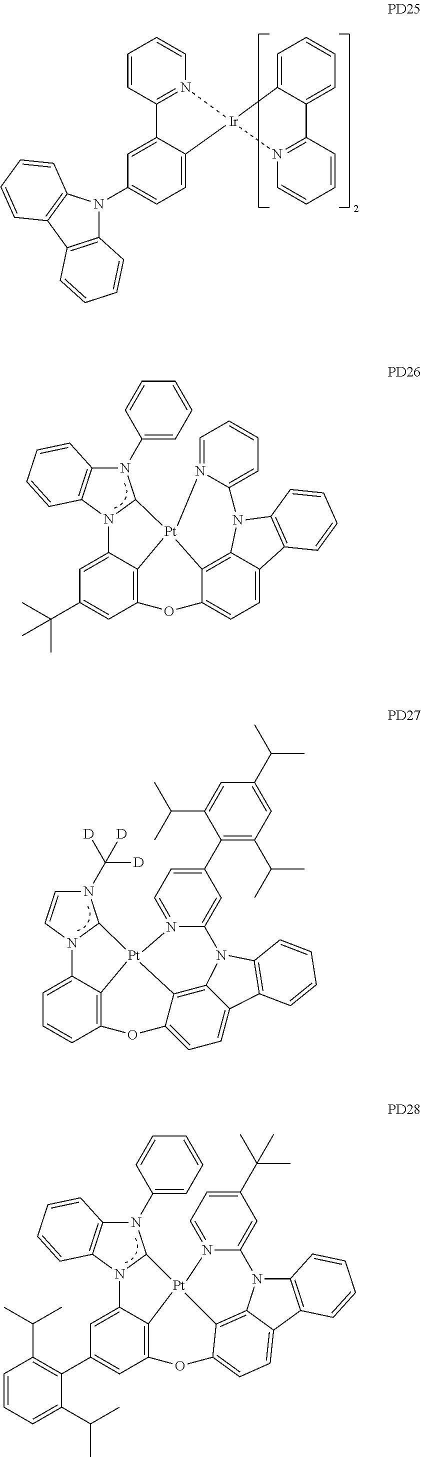

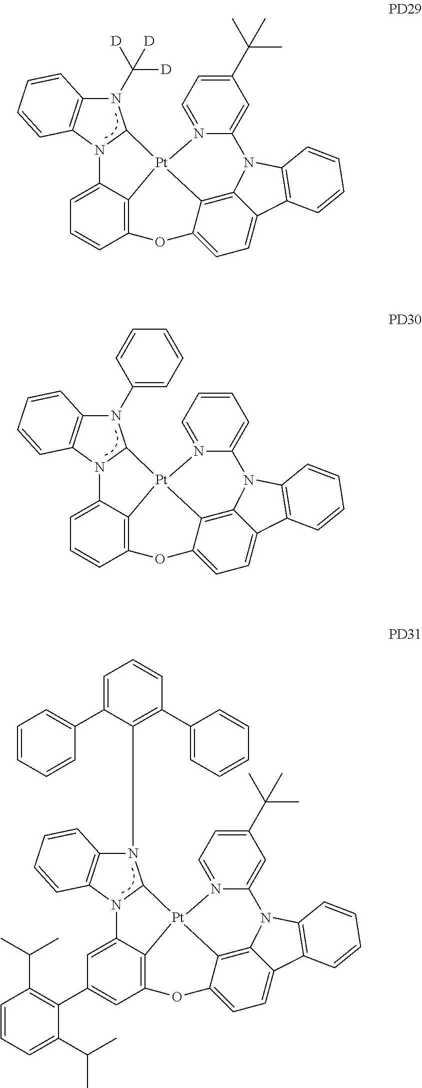

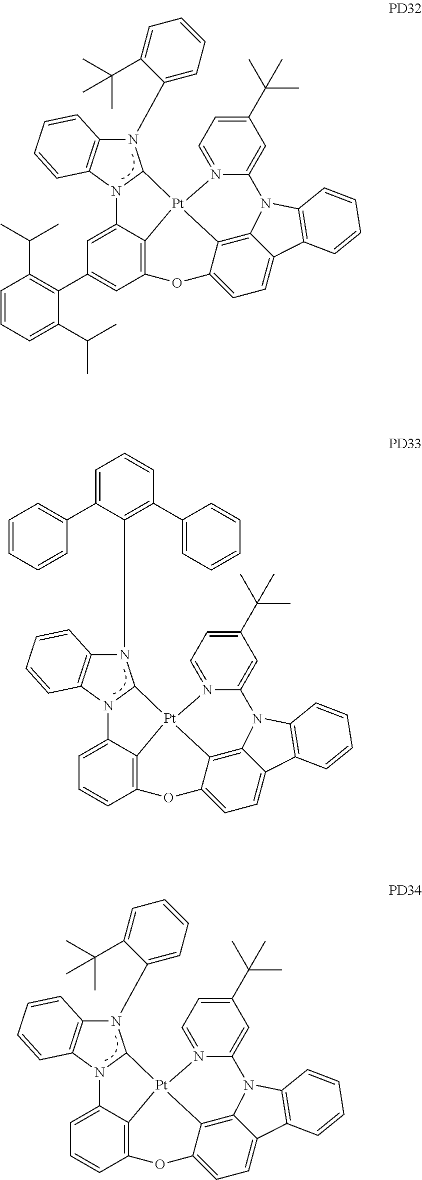

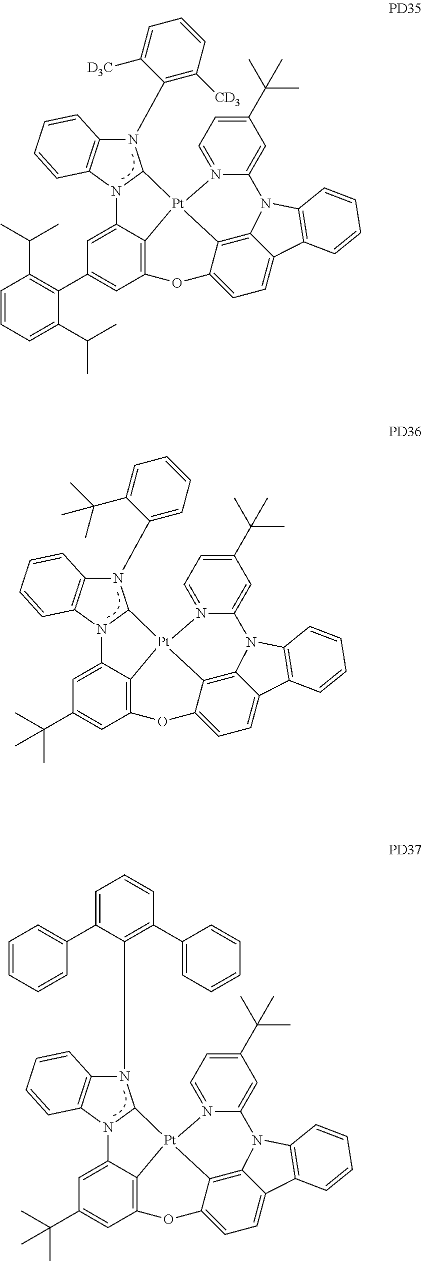

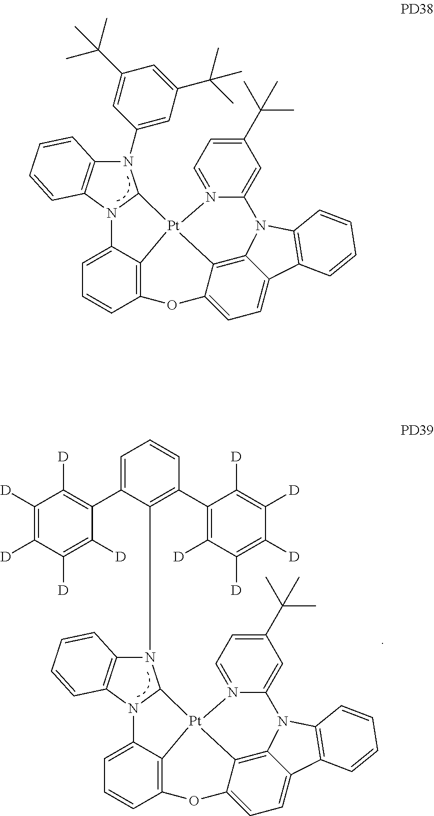

### [Fluorescent Dopant]

The emission layer may include a fluorescent dopant and/or an auxiliary dopant.

In an embodiment, the fluorescent dopant and/or the auxiliary dopant may each independently include a compound represented by Formula 501:

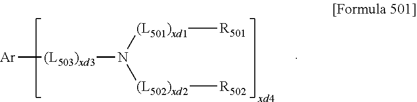

In Formula 501,
Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xd1 to xd3 may each independently be 0, 1, 2, or 3, and
xd4 may be 1, 2, 3, 4, 5, or 6.

In an embodiment, in Formula 501, Ar₅₀₁ may be a condensed cyclic group (for example, an anthracene group, a chrysene group, a pyrene group, etc.) in which three or more monocyclic groups are condensed together.

In an embodiment, in Formula 501, xd4 may be 2.

In an embodiment, the fluorescent dopant and the auxiliary dopant may each include one of Compounds FD1 to FD38, DPVBi, DPAVBi, or any combination thereof:

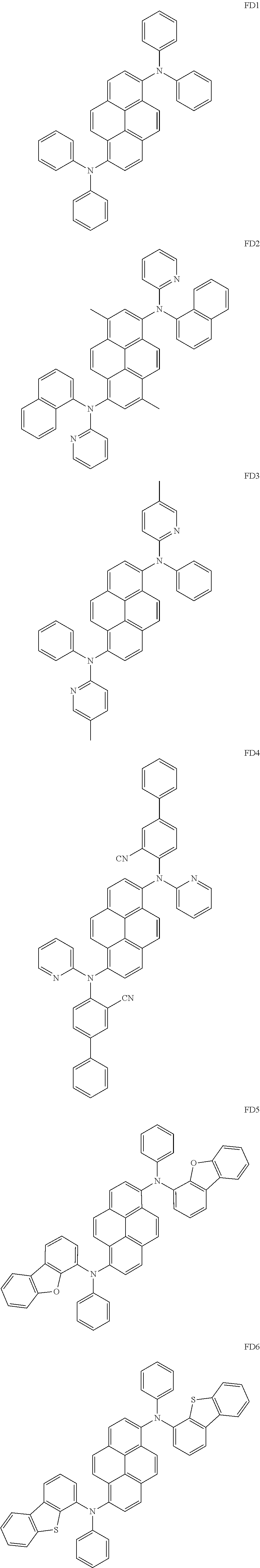

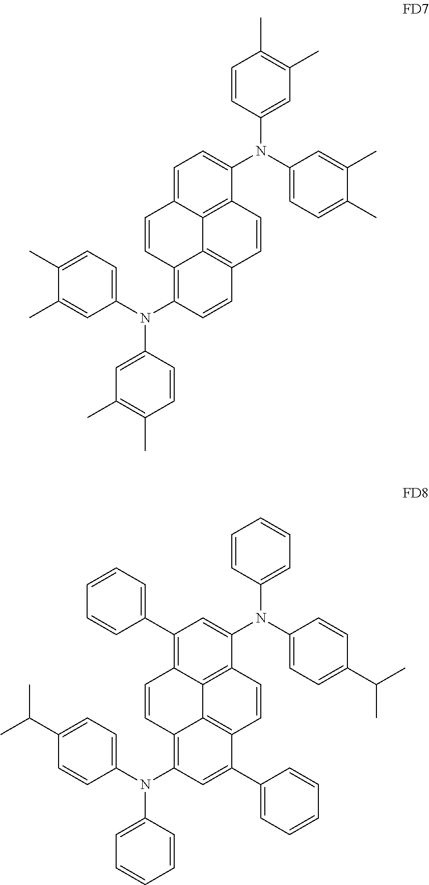

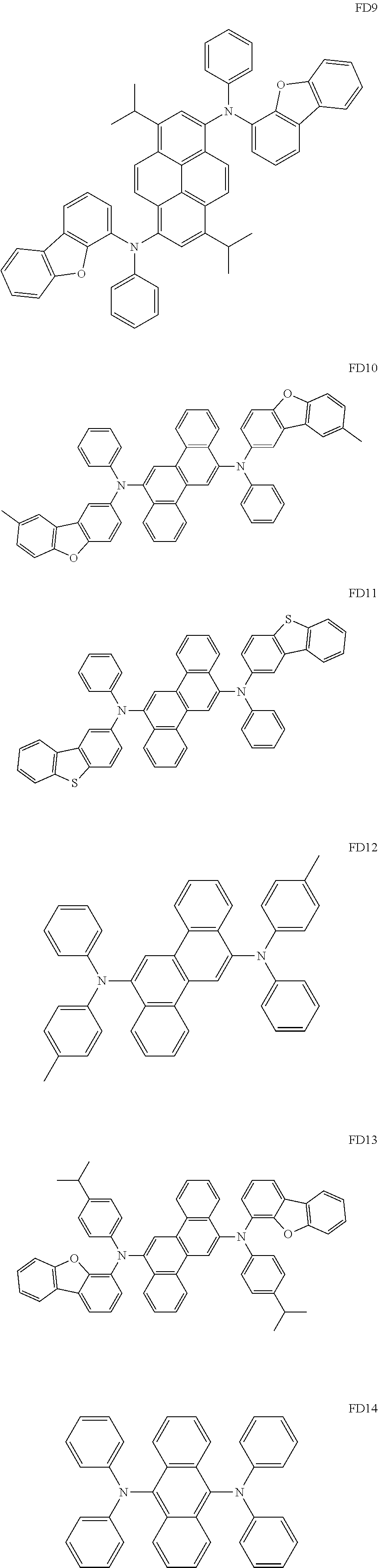

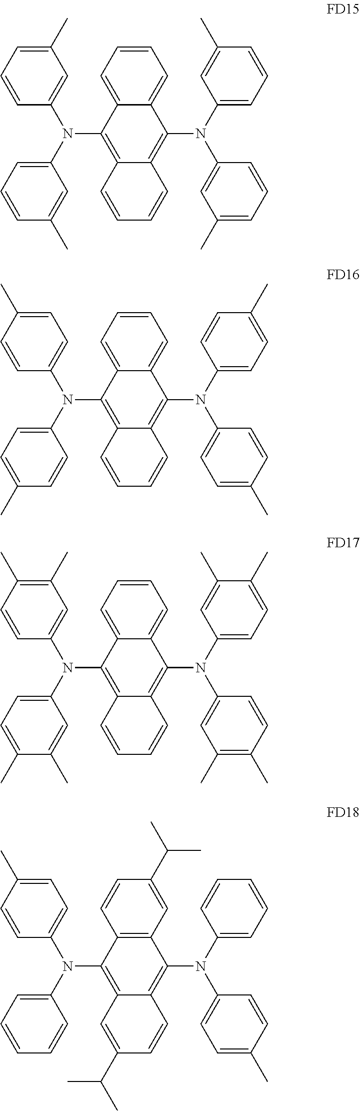

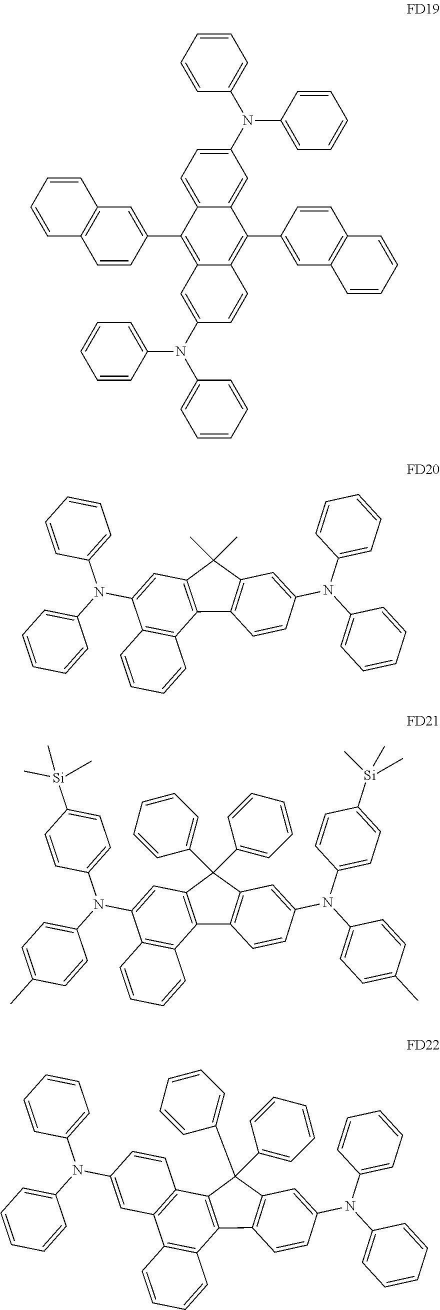

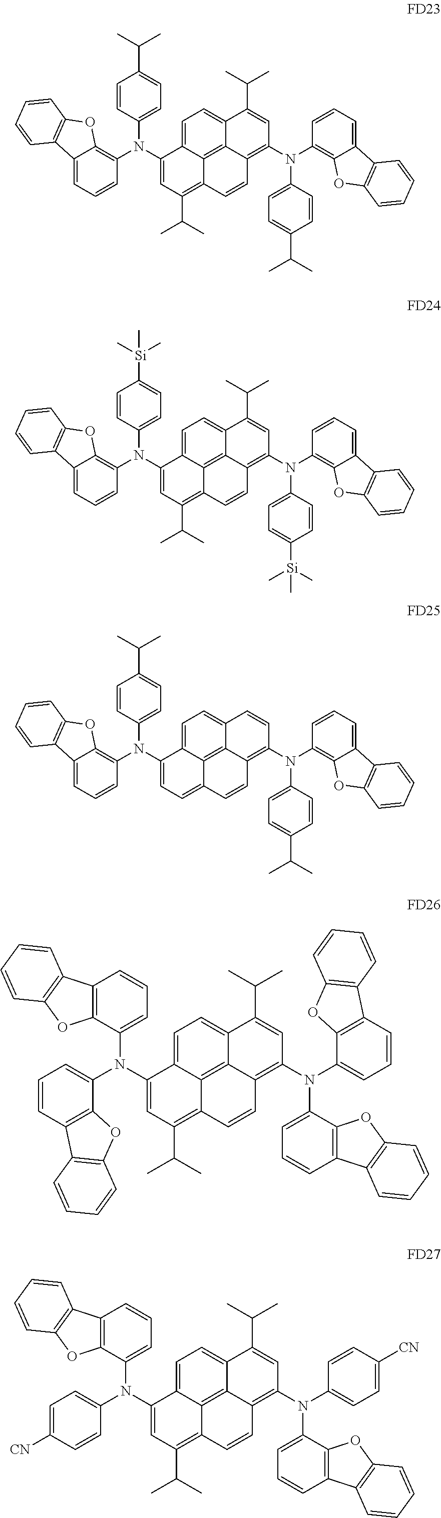

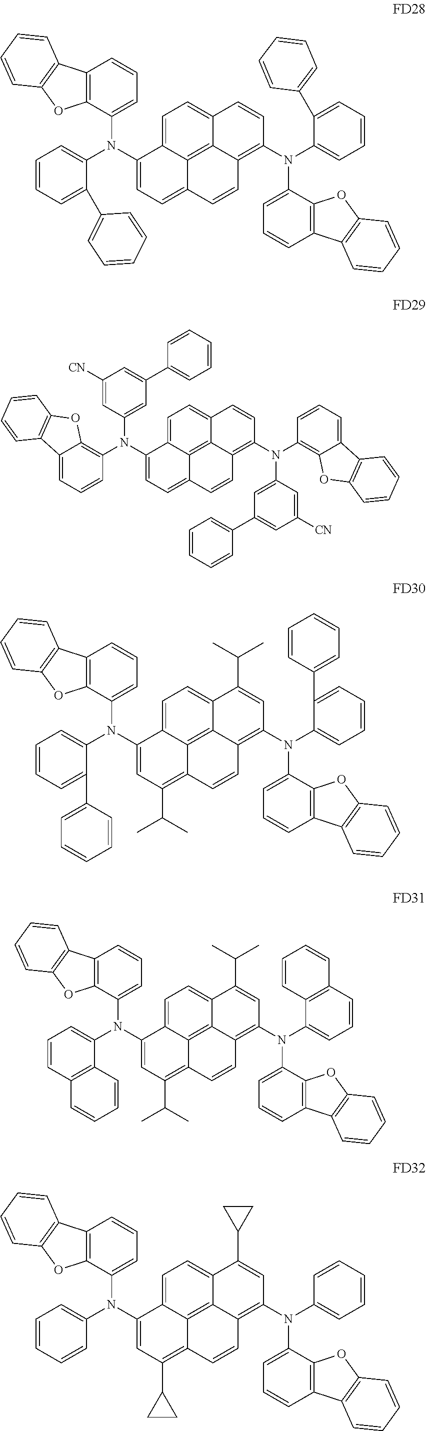

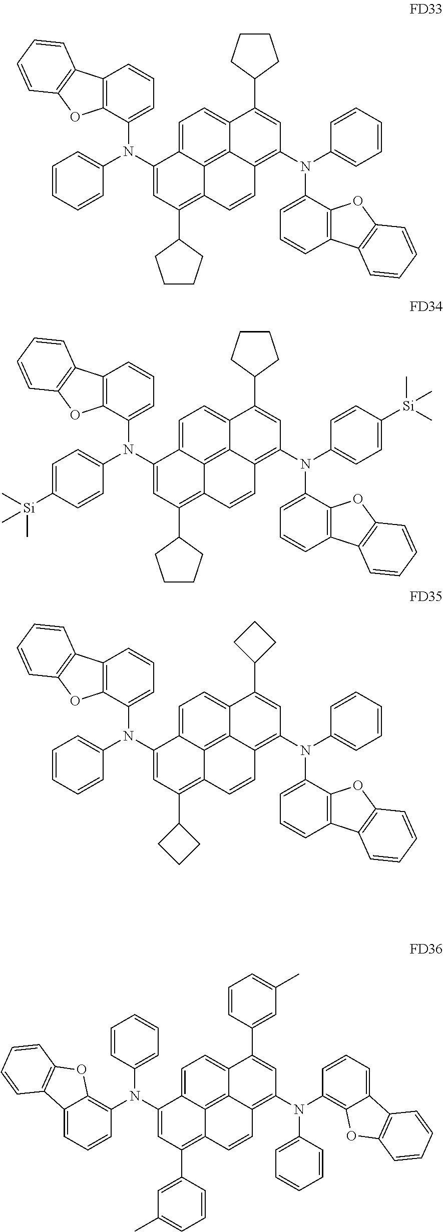

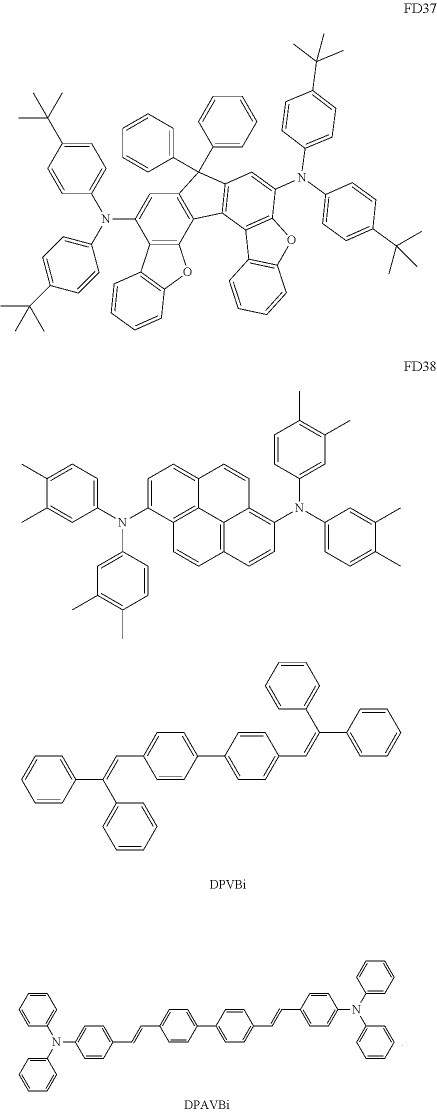

### [Delayed Fluorescence Material]

The emission layer may include a delayed fluorescence material.

In the specification, the delayed fluorescence material may be selected from compounds capable of emitting delayed fluorescence, based on a delayed fluorescence emission mechanism.

The delayed fluorescence material included in the emission layer may serve as a host or as a dopant, depending on the types of other materials included in the emission layer.

In an embodiment, a difference between a triplet energy level (eV) of the delayed fluorescence material and a singlet energy level (eV) of the delayed fluorescence material may be in a range of about 0 eV to about 0.5 eV. When a difference between a triplet energy level (eV) of the delayed fluorescence material and a singlet energy level (eV) of the delayed fluorescence material satisfies the range above, up-conversion from the triplet state to the singlet state of the delayed fluorescence materials may effectively occur, and thus, the light-emitting device 10 may have improved luminescence efficiency.

In embodiments, the delayed fluorescence material may include: a material including at least one electron donor (for example, a π electron-rich C₃-C₆₀ cyclic group and the like, such as a carbazole group) and at least one electron acceptor (for example, a sulfoxide group, a cyano group, a π electron-deficient nitrogen-containing C₁-C₆₀ cyclic group, and the like); or a material including a C₈-C₆₀ polycyclic group including at least two cyclic groups condensed to each other while sharing boron (B); or the like.

Examples of a delayed fluorescence material may include at least one of Compounds DF1 to DF14:

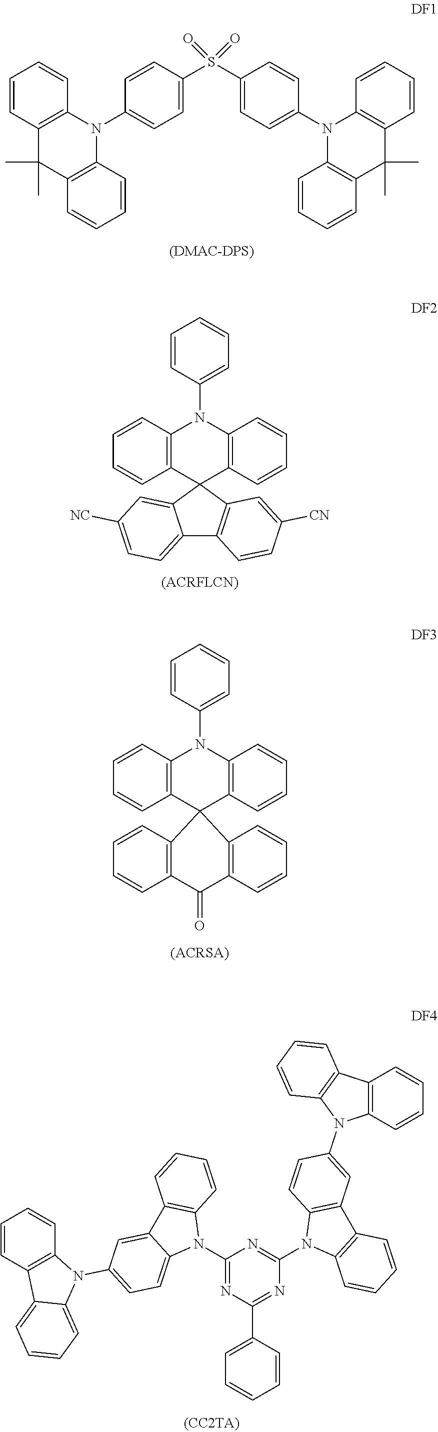

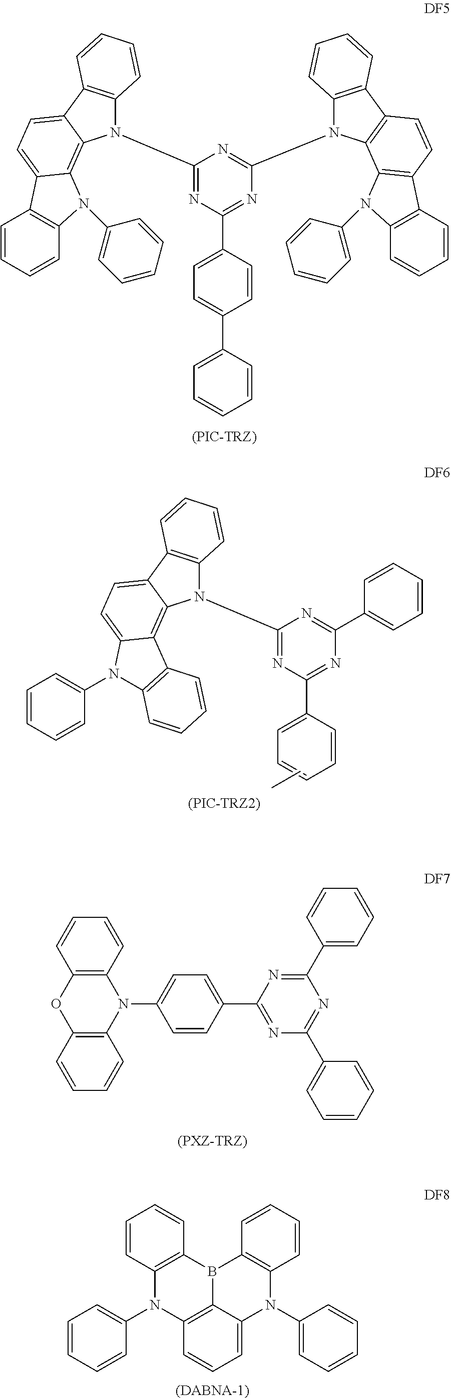

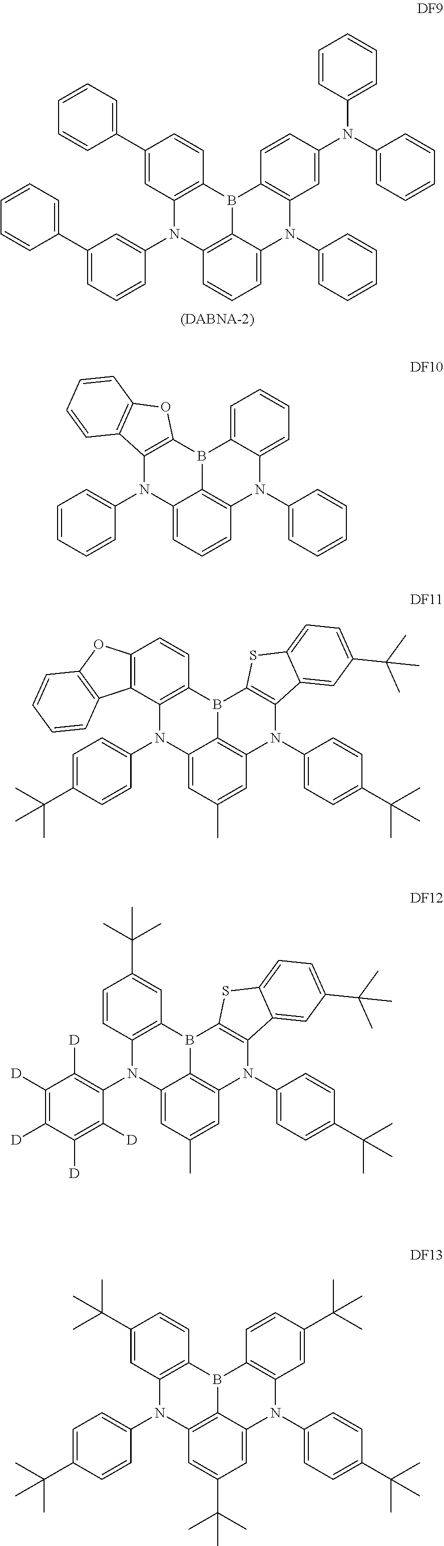

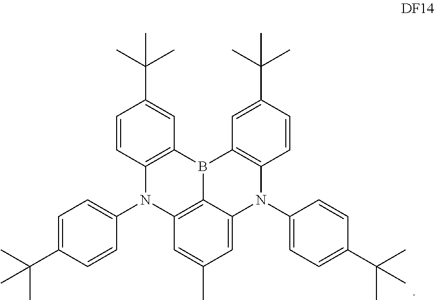 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1]

### [Quantum Dot]

The emission layer may include a quantum dot.

In the specification, a quantum dot may be a crystal of a semiconductor compound, and may include any material capable of emitting light of various emission wavelengths according to a size of the crystal. Quantum dots may emit light of various emission wavelengths by adjusting a ratio of elements included in the quantum dot compounds.

For example, a diameter of the quantum dot may be in a range of about 1 nm to about 10 nm.

The quantum dot may be synthesized by a wet chemical process, a metal organic chemical vapor deposition process, a molecular beam epitaxy process, or any process similar thereto.

The wet chemical process is a method that includes mixing a precursor material with an organic solvent and growing quantum dot particle crystals. When the crystal grows, the organic solvent naturally acts as a dispersant coordinated on the surface of the quantum dot crystal and controls the growth of the crystal so that the growth of quantum dot particles can be controlled through a process which costs less, and may be more readily performed than vapor deposition methods, such as metal organic chemical vapor deposition (MOCVD) or molecular beam epitaxy (MBE).

The quantum dot may include a Group II-VI semiconductor compound, a Group III-V semiconductor compound, a Group III-VI semiconductor compound, a Group I-III-VI semiconductor compound, a Group IV-VI semiconductor compound, a Group IV element or compound, or any combination thereof.

Examples of a Group II-VI semiconductor compound may include: a binary compound, such as CdS, CdSe, CdTe, ZnS, ZnSe, ZnTe, ZnO, HgS, HgSe, HgTe, MgSe, MgS, and the like; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and the like; a quaternary compound, such as CdZnSeS, CdZnSeTe, CdZnSTe, CdHgSeS, CdHgSeTe, CdHgSTe, HgZnSeS, HgZnSeTe, HgZnSTe, and the like; or any combination thereof.

Examples of a Group III-V semiconductor compound may include: a binary compound, such as GaN, GaP, GaAs, GaSb, AlN, AIP, AIAs, AISb, InN, InP, InAs, InSb, or the like; a ternary compound, such as GaNP, GaNAs, GaNSb, GaPAs, GaPSb, AlNP, AlNAs, AlNSb, AIPAs, AIPSb, InGaP, InNP, InAlP, InNAs, InNSb, InPAs, InPSb, GaAINP, or the like; a quaternary compound, such as GaAINAs, GaAINSb, GaAIPAs, GaAIPSb, GalnNP, GalnNAs, GalnNSb, GalnPAs, GalnPSb, InAlNP, InAlNAs, InAlNSb, InAlPAs, InAlPSb, or the like; or any combination thereof. In an embodiment, the Group III-V semiconductor compound may further include a Group II element. Examples of a Group III-V semiconductor compound further including the Group II element may include InZnP, InGaZnP, InAlZnP, and the like.

Examples of a Group III-VI semiconductor compound may include: a binary compound, such as GaS, GaSe, Ga₂Se₃, GaTe, InS, InSe, In₂S₃, In₂Se₃, InTe, and the like; a ternary compound, such as InGaS₃, InGaSe₃, and the like; or any combination thereof.

Examples of a Group I-III-VI semiconductor compound may include: a ternary compound, such as AgInS, AgInS₂, AgInSe₂, AgGaS, AgGaS₂, AgGaSe₂, CuInS, CuInS₂, CuInSe₂, CuGaS₂, CuGaSe₂, CuGaO₂, AgGaO₂, AgAlO₂, etc.; a quaternary compound, such as AgInGaS₂, AgInGaSe₂, etc.; or any combination thereof.

Examples of a Group IV-VI semiconductor compound may include: a binary compound, such as SnS, SnSe, SnTe, PbS, PbSe, PbTe, and the like; a ternary compound, such as SnSeS, SnSeTe, SnSTe, PbSeS, PbSeTe, PbSTe, SnPbS, SnPbSe, SnPbTe, and the like; a quaternary compound, such as SnPbSSe, SnPbSeTe, SnPbSTe, and the like; or any combination thereof.

Examples of a Group IV element or compound may include: a single element material, such as Si, Ge, and the like; a binary compound, such as SiC, SiGe, and the like; or any combination thereof.

Each element included in a multi-element compound, such as a binary compound, a ternary compound, and a quaternary compound, may be present in a particle at a uniform concentration or at a non-uniform concentration. For example, the formulae above refer to types of elements included in the compound, wherein the ratios of elements in the compound may vary. For example, AgInGaS₂ refers to AgInₓGa₁₋ₓS₂ (where x is a real number between 0 and 1).

In an embodiment, the quantum dot may have a single structure in which the concentration of each element in the quantum dot is uniform, or the quantum dot may have a core-shell structure. In an embodiment, in case that the quantum dot has a core-shell structure, a material included in the core and a material included in the shell may be different from each other.

The shell of the quantum dot may serve as a protective layer which prevents chemical denaturation of the core to maintain semiconductor characteristics, and/or may serve as a charging layer that imparts electrophoretic characteristics to the quantum dot. The shell may be single-layered or multi-layered. An interface between the core and the shell may have a concentration gradient in which the concentration of a material that is present in the shell decreases toward the core.

Examples of a shell of the quantum dot may include a metal oxide, a metalloid oxide, a non-metal oxide, a semiconductor compound, or a combination thereof. Examples of a metal oxide, a metalloid oxide, or a non-metal oxide may include: a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, NiO, and the like; a ternary compound, such as MgAl₂O₄, CoFe₂O₄, NiFe₂O₄, CoMn₂O₄, and the like; or any combination thereof.

Examples of a semiconductor compound may include, as described herein: a Group II-VI semiconductor compound; a Group III-V semiconductor compound; a Group III-VI semiconductor compound; a Group I-III-VI semiconductor compound; a Group IV-VI semiconductor compound; or any combination thereof. Examples of a semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AIP, AISb, or any combination thereof.

The quantum dot may have a full width at half maximum (FWHM) of an emission wavelength spectrum in a range of about 45 nm to about 40 nm. For example, the quantum dot may have an FWHM of the emission wavelength spectrum equal to or less than 30 nm. When the FWHM of the quantum dot is within these ranges, the quantum dot may have improved color purity or improved color reproducibility. In addition, since light emitted through the quantum dot is emitted in all directions, the wide viewing angle may be improved.

In embodiments, the quantum dot may be in the form of a spherical particle, a pyramidal particle, a multi-arm particle, a cubic nanoparticle, a nanotube, a nanowire, a nanofiber, or a nanoplate particle.

Since the energy band gap may be adjusted by controlling the size of the quantum dot, light having various wavelength bands may be obtained from the quantum dot emission layer. Accordingly, by using quantum dots of different sizes, a light-emitting device that emits light of various wavelengths may be implemented. For example, the control of the size of the quantum dots or the ratio of elements in the quantum dot compound may be selected to emit red light, green light, and/or blue light. In embodiments, the size of the quantum dot may emit white light by a combination of light of various colors.

### [Electron Transport Region in Interlayer 130]

The electron transport region may have a structure consisting of a layer consisting of a single material, a structure consisting of a layer including different materials, or a structure including multiple layers including different materials.

The electron transport region may include a buffer layer, a hole blocking layer, an electron control layer, an electron transport layer, an electron injection layer, or any combination thereof.

In embodiments, the electron transport region may have an electron transport layer/electron injection layer structure, a hole blocking layer/electron transport layer/electron injection layer structure, an electron control layer/electron transport layer/electron injection layer structure, or a buffer layer/electron transport layer/electron injection layer structure, wherein the layers of each structure may be stacked from an emission layer in its respective stated order, but the structure of the electron transport region is not limited thereto.

The electron transport region (e.g., a buffer layer, a hole blocking layer, an electron control layer, or an electron transport layer in the electron transport region) may include a metal-free compound including at least one IT electron-deficient nitrogen-containing C₁-C₆₀ heterocyclic group.

In embodiments, the electron transport region may include a compound represented by Formula 601:

[Ar₆₀₁]ₓₑ₁₁-[(L₆₀₁)ₓₑ₁-R₆₀₁]ₓₑ₂₁ [Formula 601]

In Formula 601,
Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ may each independently be the same as described in connection with Q₁,
xe21 may be 1, 2, 3, 4, or 5, and
at least one of Ar₆₀₁, L₆₀₁, and R₆₀₁ may each independently be a π electron-deficient nitrogen-containing C₁-C₆₀ cyclic group that is unsubstituted or substituted with at least one R₁₀ₐ.

In an embodiment, in Formula 601, when xe11 is 2 or more, two or more of Ar₆₀₁ may be linked to each other via a single bond.

In an embodiment, in Formula 601, Ar₆₀₁ may be an anthracene group substituted or unsubstituted at least one R₁₀ₐ.

In an embodiment, the electron transport region may include a compound represented by Formula 601-1:

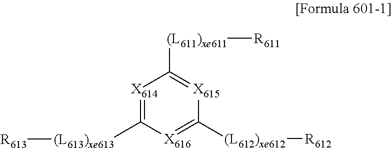

In Formula 601-1,
X₆₁₄ may be N or C(R₆₁₄), X₆₁₅ may be N or C(R₆₁₅), X₆₁₆ may be N or C(R₆₁₆), and at least one of X₆₁₄ to X₆₁₆ may each be N,
L₆₁₁ to L₆₁₃ may each independently be the same as described in connection with L₆₀₁,
xe611 to xe613 may each independently be the same as described in connection with xe1,
R₆₁₁ to R₆₁₃ may each independently be the same as described in connection with R₆₀₁, and
R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

In embodiments, in Formulae 601 and 601-1, xe1 and xe611 to xe613 may each independently be 0, 1, or 2.

In embodiments, the electron transport region may include: one of Compounds ET1 to ET45; 2,9-dimethyl-4,7-diphenyl-1,10-phenanthroline (BCP); 4,7-diphenyl-1,10-phenanthroline (Bphen); Alq3; BAlq; TAZ; NTAZ; or any combination thereof:

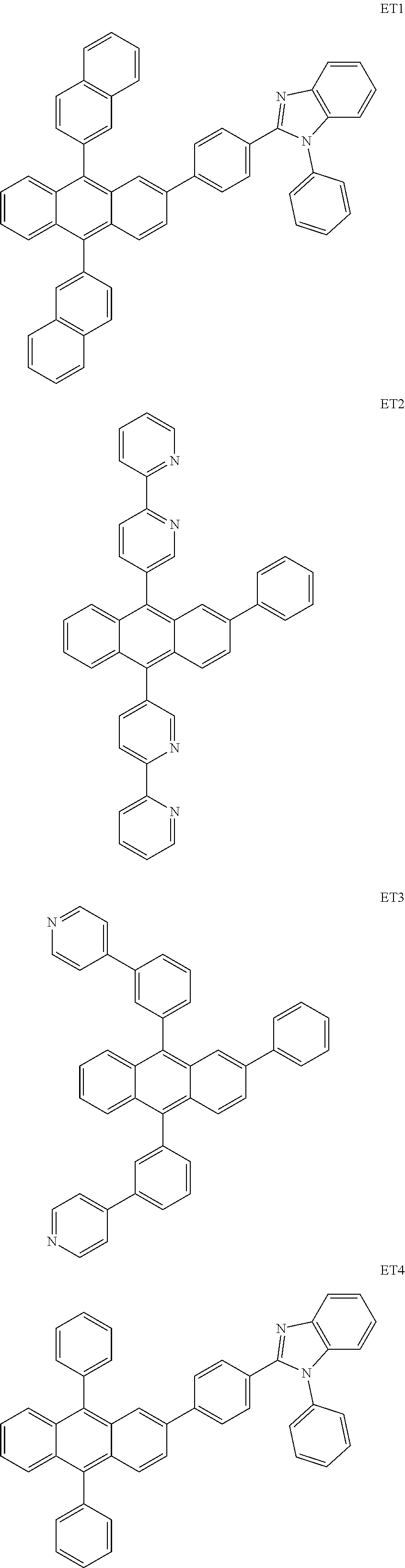 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1 ; c1ccc(-c2c3ccccc3c(-c3ccccc3)c3cc(-c4ccc(-c5nc6ccccc6n5-c5ccccc5)cc4)ccc23)cc1]

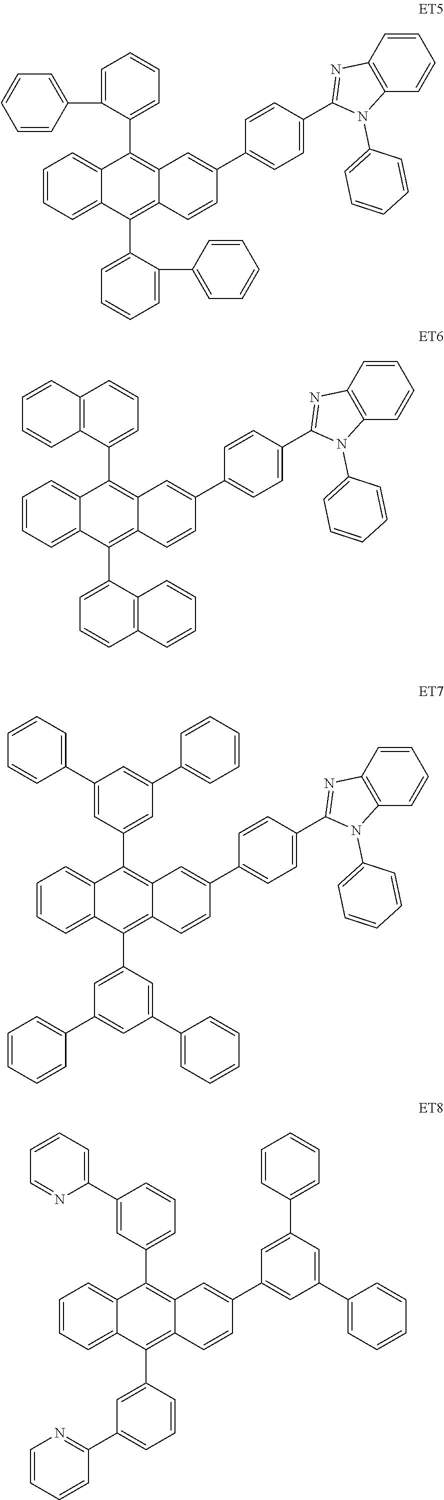 [SMILES: c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1]

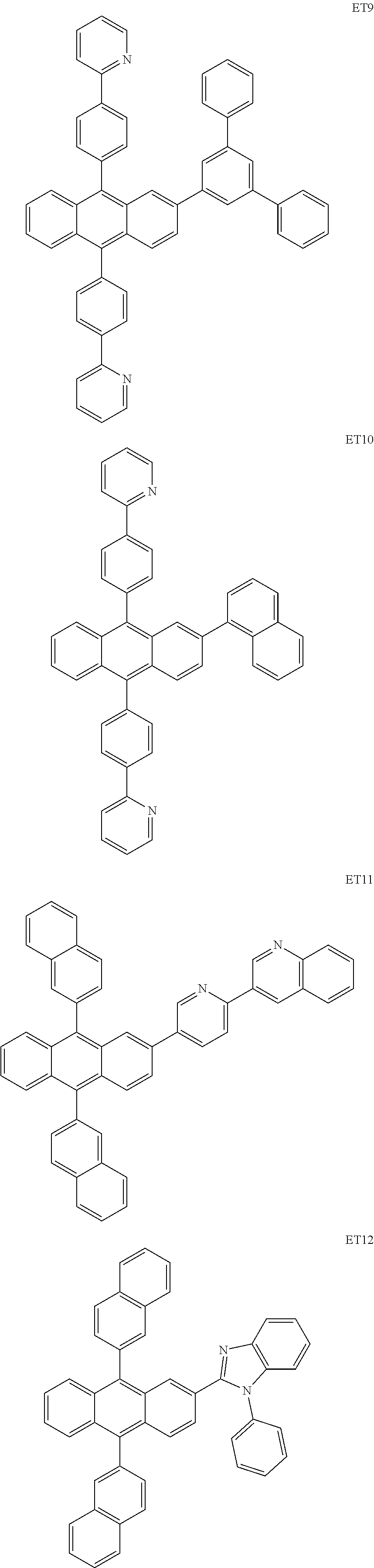

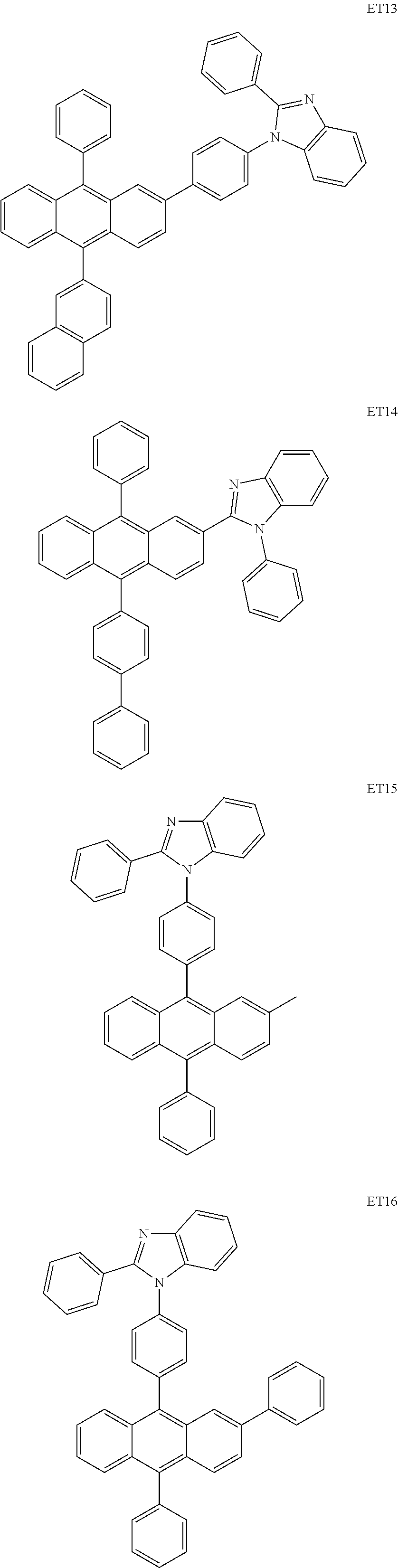 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

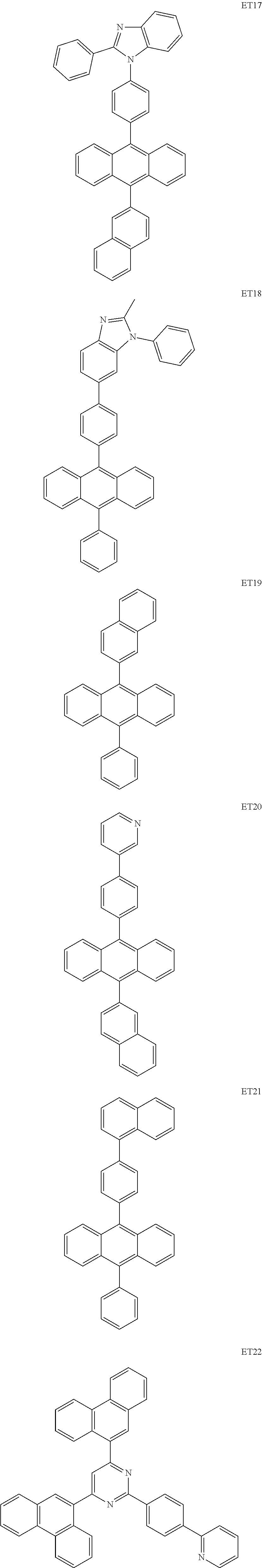

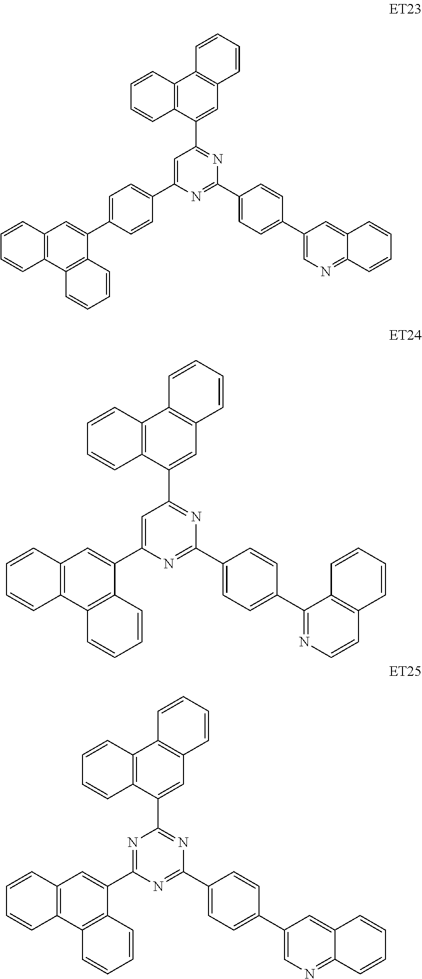 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

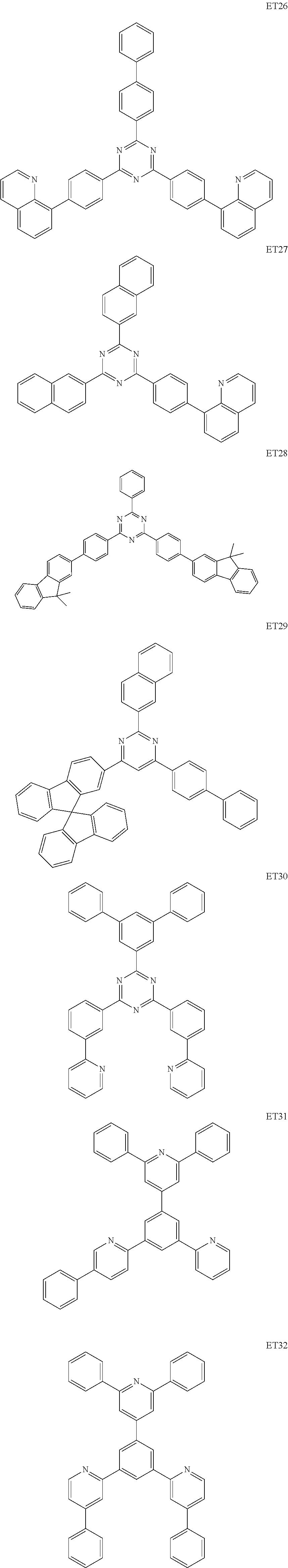

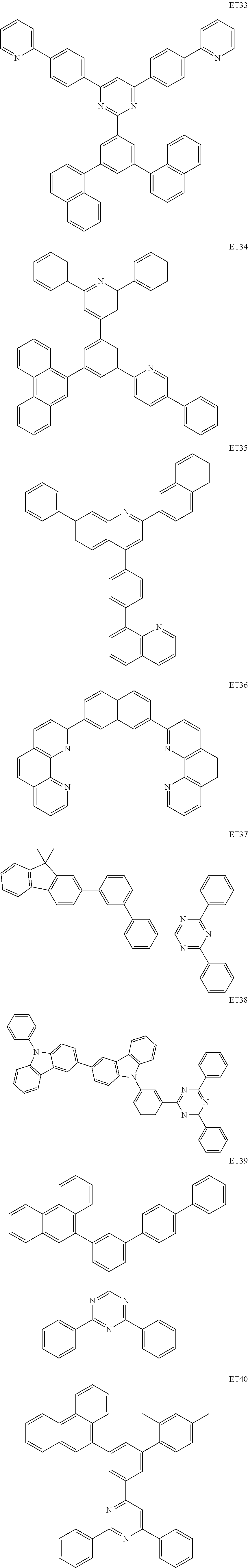

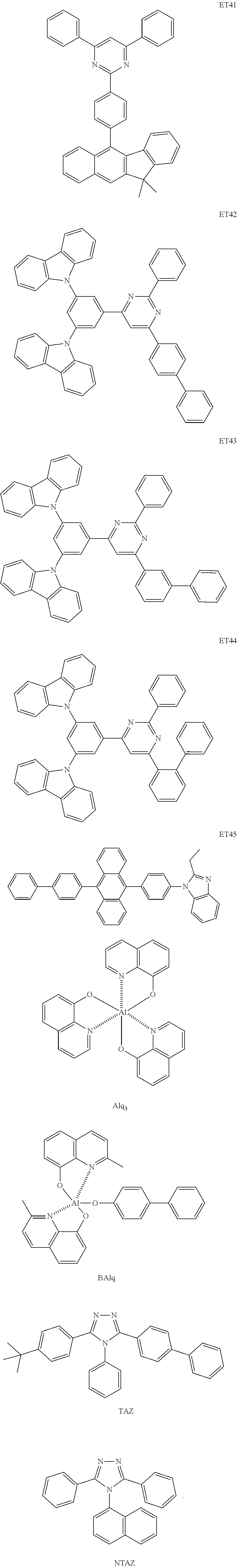

A thickness of the electron transport region may be in a range of about 100 Å to about 5,000 Å. For example, the thickness of the electron transport region may be in a range of about 160 Å to about 4,000 Å. When the electron transport region includes a buffer layer, a hole blocking layer, an electron control layer, an electron transport layer, or any combination thereof, a thickness of the buffer layer, the hole blocking layer, or the electron control layer may each independently be in a range of about 20 Å to about 1,000 Å, and a thickness of the electron transport layer may be in a range of about 100 Å to about 1,000 Å. For example, the thickness of the buffer layer, the hole blocking layer, or the electron control layer may each independently be in a range of about 30 Å to about 300 Å. For example, the thickness of the electron transport layer may be in a range of about 150 Å to about 500 Å. When the thicknesses of the buffer layer, the hole blocking layer, the electron control layer, the electron transport layer, and/or the electron transport region are within these ranges, satisfactory electron transporting characteristics may be obtained without a substantial increase in driving voltage.

The electron transport region (for example, an electron transport layer in the electron transport region) may further include, in addition to the materials described above, a metal-containing material.

The metal-containing material may include an alkali metal complex, an alkaline earth metal complex, or any combination thereof. A metal ion of an alkali metal complex may be a Li ion, a Na ion, a K ion, a Rb ion, or a Cs ion, and a metal ion of an alkaline earth metal complex may be a Be ion, a Mg ion, a Ca ion, a Sr ion, or a Ba ion.

A ligand coordinated with the metal ion of the alkali metal complex or with the metal ion of the alkaline earth-metal complex may each independently include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or any combination thereof.

For example, the metal-containing material may include a Li complex. The Li complex may include, for example, Compound ET-D1 (LiQ) or Compound ET-D2:

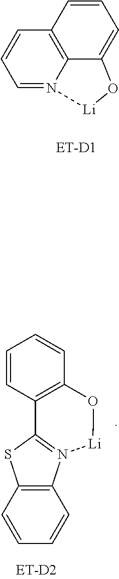

The electron transport region may include an electron injection layer that facilitates the injection of electrons from the second electrode 150. The electron injection layer may directly contact the second electrode 150.

The electron injection layer may have a structure consisting of a layer consisting of a single material, a structure consisting of a layer including different materials, or a structure including multiple layers including different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may be oxides, halides (for example, fluorides, chlorides, bromides, iodides, etc.), or tellurides of the alkali metal, the alkaline earth metal, and the rare earth metal, or any combination thereof.

The alkali metal-containing compound may include: an alkali metal oxide, such as Li₂O, Cs₂O, K₂O, etc.; an alkali metal halide, such as LiF, NaF, CsF, KF, LiI, NaI, CsI, KI, etc.; or any combination thereof. The alkaline earth metal-containing compound may include an alkaline earth metal compound, such as BaO, SrO, CaO, BaₓSr₁₋ₓO (wherein x is a real number satisfying 0<x<1), BaₓCa₁₋ₓO (wherein x is a real number satisfying 0<x<1), and the like. The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In an embodiment, the rare earth metal-containing compound may include a lanthanide metal telluride. Examples of a lanthanide metal telluride may include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, Lu₂Te₃, and the like.

The alkali metal complex, the alkaline earth-metal complex, and the rare earth metal complex may include: an alkali metal ion, an alkaline earth metal ion, or a rare earth metal ion; and a ligand bonded to the metal ion (for example, a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenyl benzimidazole, a hydroxyphenylbenzothiazole, a bipyridine, a phenanthroline, a cyclopentadiene, or any combination thereof).

In an embodiment, the electron injection layer may consist of an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In embodiments, the electron injection layer may further include an organic material (for example, a compound represented by Formula 601).

In an embodiment, the electron injection layer may consist of an alkali metal-containing compound (for example, alkali metal halide); or the electron injection layer may consist of an alkali metal-containing compound (for example, alkali metal halide), and an alkali metal, an alkaline earth metal, a rare earth metal, or any combination thereof. For example, the electron injection layer may be a KI:Yb co-deposited layer, an RbI:Yb co-deposited layer, a LiF:Yb co-deposited layer, and the like.

When the electron injection layer further includes an organic material, an alkali metal, an alkaline earth metal, a rare earth metal, an alkali metal-containing compound, an alkaline earth metal-containing compound, a rare earth metal-containing compound, an alkali metal complex, an alkaline earth-metal complex, a rare earth metal complex, or any combination thereof may be uniformly or non-uniformly dispersed in a matrix including the organic material.

A thickness of the electron injection layer may be in a range of about 1 Å to about 100 Å. For example, a thickness of the electron injection layer may be in a range of about 3 Å to about 90 Å. When the thickness of the electron injection layer is within any of these ranges, satisfactory electron injection characteristics may be obtained without a substantial increase in driving voltage.

### [Second Electrode 150]

The second electrode 150 is arranged on the interlayer 130 having the aforementioned structure. The second electrode 150 may be a cathode, which is an electron injection electrode. A material for forming the second electrode 150 may be a material having a low work function, such as a metal, an alloy, an electrically conductive compound, or any combination thereof.

The second electrode 150 may include Li, Ag, Mg, Al, Al—Li, Ca, Mg—In, Mg—Ag, Yb, Ag—Yb, ITO, IZO, or any combination thereof. The second electrode 150 may be a transmissive electrode, a semi-transmissive electrode, or a reflective electrode.

The second electrode 150 may have a single-layer structure or a multi-layer structure.

### [Capping Layer]

The light-emitting device 10 may include a first capping layer outside the first electrode 110, and/or a second capping layer outside the second electrode 150. For example, the light-emitting device 10 may have a structure in which the first capping layer, the first electrode 110, the interlayer 130, and the second electrode 150 are stacked in this stated order, a structure in which the first electrode 110, the interlayer 130, the second electrode 150, and the second capping layer are stacked in this stated order, or a structure in which the first capping layer, the first electrode 110, the interlayer 130, the second electrode 150, and the second capping layer are stacked in this stated order.

Light generated in an emission layer in the interlayer 130 of the light-emitting device 10 may be extracted toward the outside through the first electrode 110, which may be a semi-transmissive electrode or a transmissive electrode, and through the first capping layer. Light generated in an emission layer in the interlayer 130 of the light-emitting device 10 may be extracted toward the outside through the second electrode 150, which may be a semi-transmissive electrode or a transmissive electrode, and through the second capping layer.

The first capping layer and the second capping layer may each increase external emission efficiency according to the principle of constructive interference. Accordingly, the light extraction efficiency of the light-emitting device 10 is increased, so that the luminescence efficiency of the light-emitting device 10 may be improved.

The first capping layer and the second capping layer may each include a material having a refractive index greater than or equal to about 1.6 (with respect to a wavelength of about 589 nm).

The first capping layer and the second capping layer may each independently be an organic capping layer including an organic material, an inorganic capping layer including an inorganic material, or an organic-inorganic composite capping layer including an organic material and an inorganic material.

At least one of the first capping layer and the second capping layer may each independently include a carbocyclic compound, a heterocyclic compound, an amine group-containing compound, a porphine derivative, a phthalocyanine derivative, a naphthalocyanine derivative, an alkali metal complex, an alkaline earth metal complex, or any combination thereof. The carbocyclic compound, the heterocyclic compound, and the amine group-containing compound may be optionally substituted with a substituent including O, N, S, Se, Si, F, Cl, Br, I, or any combination thereof.

In embodiments, at least one of the first capping layer and the second capping layer may each independently include an amine group-containing compound.

In embodiments, at least one of the first capping layer and the second capping layer may each independently include a compound represented by Formula 201, a compound represented by Formula 202, or any combination thereof.

In embodiments, at least one of the first capping layer and the second capping layer may each independently include one of Compounds HT28 to HT33, one of Compounds CP1 to CP6, β-NPB, or any combination thereof:

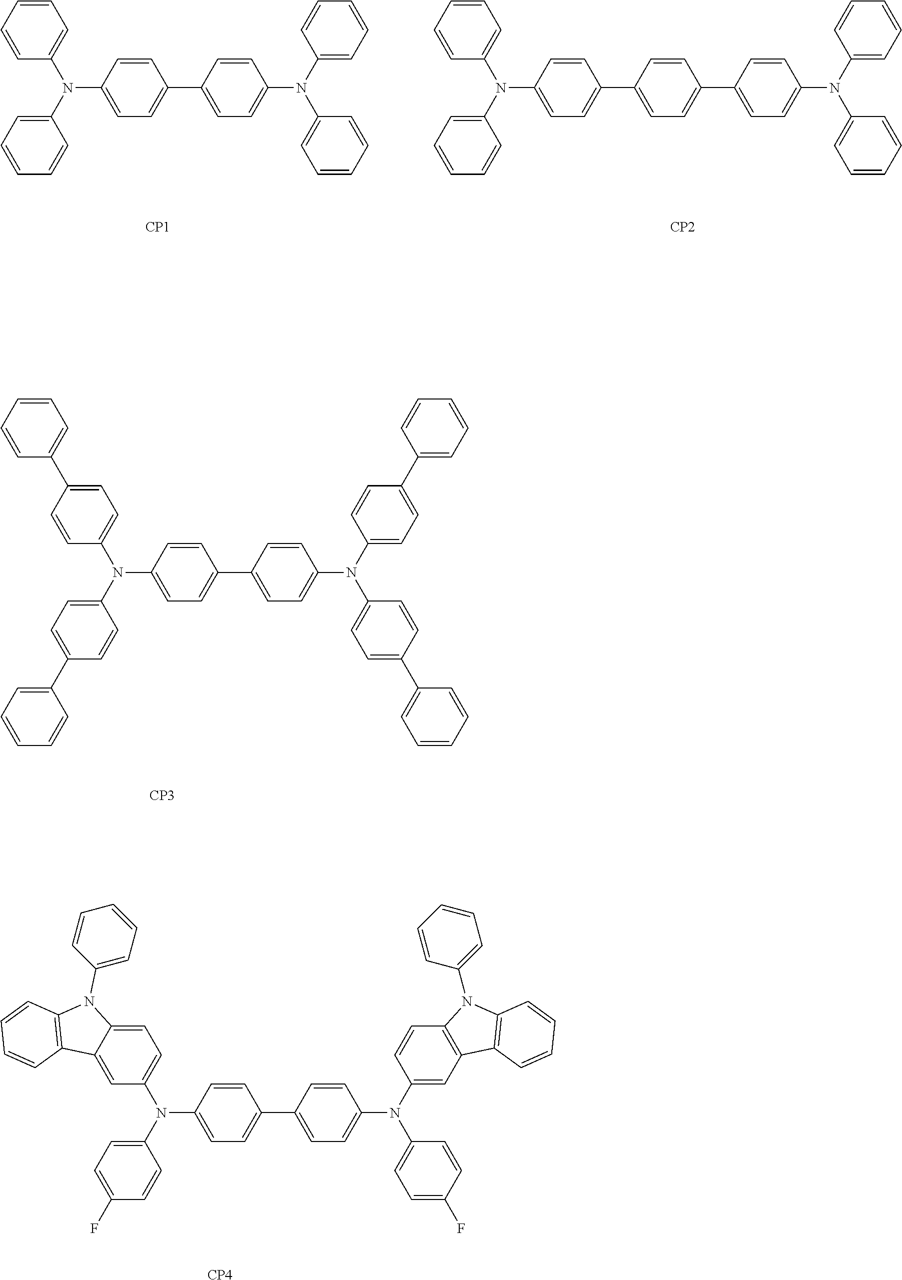

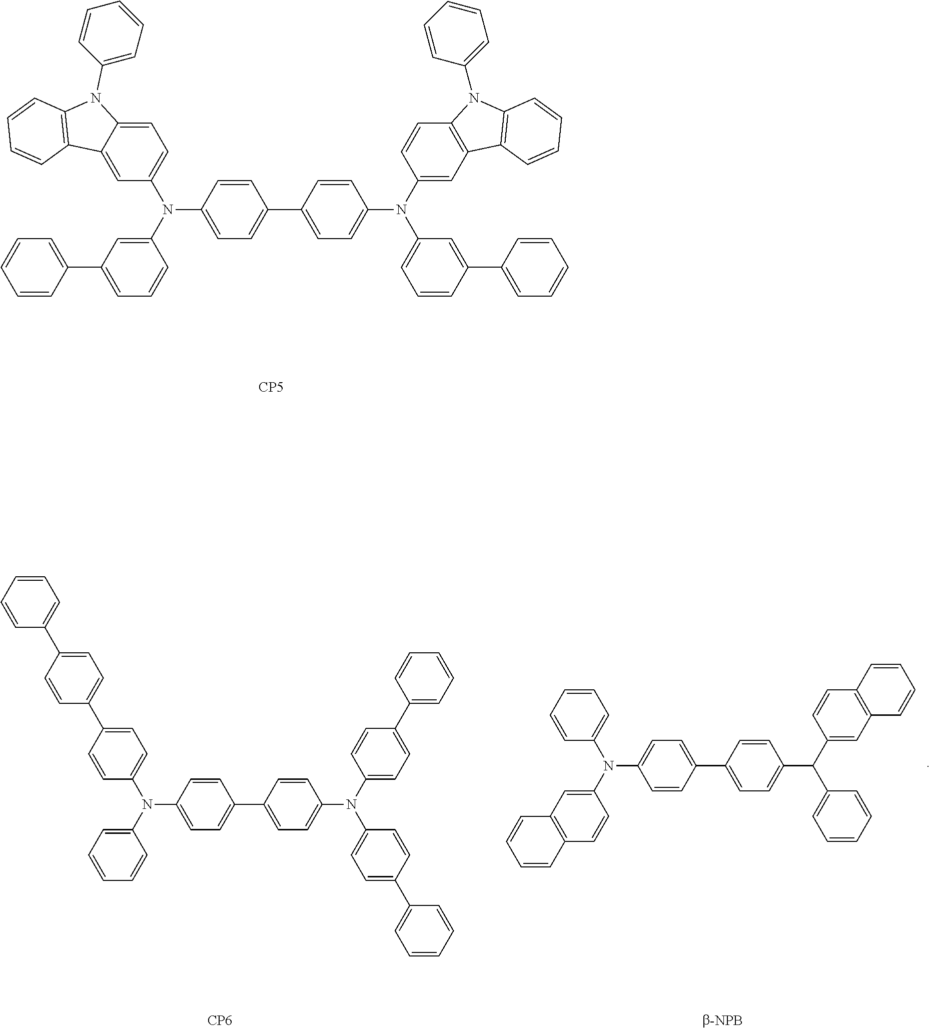 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; "CP5" ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(C(c2ccc(-c3ccc(N(c4ccccc4)c4ccc5ccccc5c4)cc3)cc2)c2ccc3ccccc3c2)cc1 ; "CP6" ; "b-NPB"]

### [Film]

An amine-based compound represented by Formula 1 may be included in various films. Accordingly, another embodiment provides a film which may include the amine-based compound represented by Formula 1. The film may be, for example, an optical member (or a light control means) (for example, a color filter, a color conversion member, a capping layer, a light extraction efficiency enhancement layer, a selective light absorbing layer, a polarizing layer, a quantum dot-containing layer, or like), a light-blocking member (for example, a light reflective layer, a light absorbing layer, or the like), or a protective member (for example, an insulating layer, a dielectric layer, or the like).

### [Electronic Apparatus]

The light-emitting device may be included in various electronic apparatuses. In embodiments, an electronic apparatus including the light-emitting device may be a light-emitting apparatus, an authentication apparatus, or the like.

The electronic apparatus (for example, a light-emitting apparatus) may further include, in addition to the light-emitting device, a color filter, a color conversion layer, or a color filter and a color conversion layer. The color filter and/or the color conversion layer may be arranged in at least one traveling direction of light emitted from the light-emitting device. In embodiments, light emitted from the light-emitting device may be blue light, green light, or white light. The light-emitting device may be the same as described herein. In an embodiment, the color conversion layer may include a quantum dot.

The electronic apparatus may include a first substrate. The first substrate may include subpixels, the color filter may include color filter areas respectively corresponding to the subpixels, and the color conversion layer may include color conversion areas respectively corresponding to the subpixels.

A pixel-defining film may be arranged between the subpixels to define each subpixel.

The color filter may further include color filter areas and light-shielding patterns arranged between the color filter areas, and the color conversion layer may further include color conversion areas and light-shielding patterns arranged between the color conversion areas.

The color filter areas (or the color conversion areas) may include a first area emitting first color light, a second area emitting second color light, and/or a third area emitting third color light, wherein the first color light, the second color light, and/or the third color light may have different maximum emission wavelengths from one another. For example, the first color light may be red light, the second color light may be green light, and the third color light may be blue light. For example, the color filter areas (or the color conversion areas) may include quantum dots. For example, the first area may include a red quantum dot, the second area may include a green quantum dot, and the third area may not include a quantum dot. The quantum dot may be a quantum dot as described herein. The first area, the second area, and/or the third area may each further include a scatterer.

In an embodiment, the light-emitting device may emit first light, the first area may absorb the first light to emit first-first color light, the second area may absorb the first light to emit second-first color light, and the third area may absorb the first light to emit third-first color light. In this regard the first-first color light, the second-first color light, and the third-first color light may have different maximum emission wavelengths from one another. For example, the first light may be blue light, the first-first color light may be red light, the second-first color light may be green light, and the third-first color light may be blue light.

The electronic apparatus may further include a thin-film transistor, in addition to the light-emitting device as described above. The thin-film transistor may include a source electrode, a drain electrode, and an active layer, wherein any one of the source electrode and the drain electrode may be electrically connected to any one of the first electrode and the second electrode of the light-emitting device.

The thin-film transistor may further include a gate electrode, a gate insulating film, and the like.

The active layer may include crystalline silicon, amorphous silicon, an organic semiconductor, an oxide semiconductor, or the like.

The electronic apparatus may further include a sealing portion for sealing the light-emitting device. The sealing portion may be arranged between the color filter and/or the color conversion layer and the light-emitting device. The sealing portion may allow light from the light-emitting device to be extracted to the outside, and may simultaneously prevent ambient air and moisture from penetrating into the light-emitting device. The sealing portion may be a sealing substrate including a transparent glass substrate or a plastic substrate. The sealing portion may be a thin-film encapsulation layer including at least one of an organic layer and/or an inorganic layer. When the sealing portion is a thin-film encapsulation layer, the electronic apparatus may be flexible.

Various functional layers may be further included on the sealing portion, in addition to the color filter and/or the color conversion layer, according to the use of the electronic apparatus. Examples of a functional layers may include a touch screen layer, a polarizing layer, and the like. The touch screen layer may be a pressure-sensitive touch screen layer, a capacitive touch screen layer, or an infrared touch screen layer. The authentication apparatus may be, for example, a biometric authentication apparatus that authenticates an individual by using biometric information of a living body (for example, fingertips, pupils, etc.).

The authentication apparatus may further include, in addition to the light-emitting device as described above, a biometric information collector.

The electronic apparatus may be applied to various displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, or endoscope displays), fish finders, various measuring instruments, meters (for example, meters for a vehicle, an aircraft, and a vessel), projectors, and the like.

### [Electronic Device]

The light-emitting device may be included in various electronic devices.

In an embodiment, an electronic device including the light-emitting device may be a flat panel display, a curved display, a computer monitor, a medical monitor, a television (TV), an advertisement board, an indoor light, an outdoor light, a signal light, a head-up display, a fully transparent display, a partially transparent display, a flexible display, a rollable display, a foldable display, a stretchable display, a laser printer, a telephone, a mobile phone, a tablet personal computer, a phablet, a personal digital assistant (PDA), a wearable device, a laptop computer, a digital camera, a camcorder, a viewfinder, a microdisplay, a three-dimensional (3D) display, a virtual reality display, an augmented reality display, a vehicle, a video wall including multiple displays tiled together, a theater screen, a stadium screen, a phototherapy device, or a sign.

Since the light-emitting device has excellent effects in terms of luminescence efficiency and long lifespan, the electronic device including the light-emitting device may have characteristics such as high luminance, high resolution, and low power consumption.

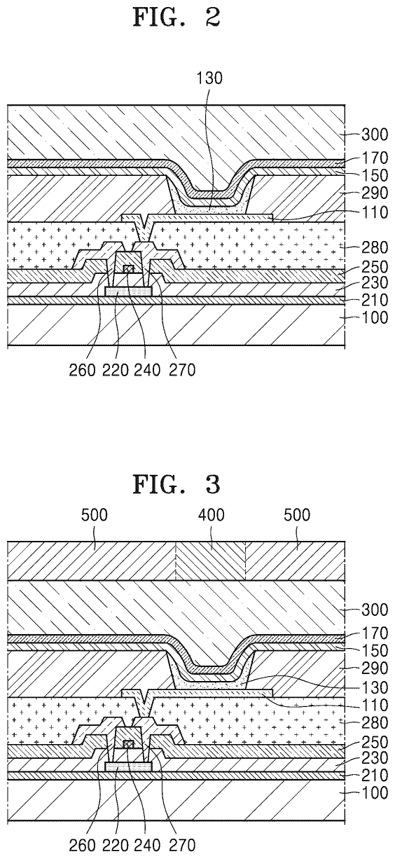

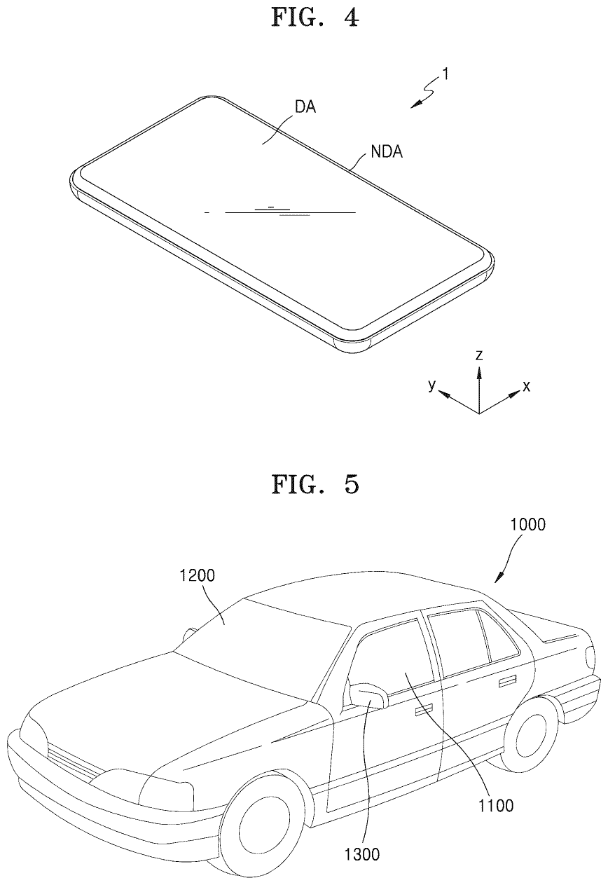

### [Description of FIGS. 2 and 3]

FIG. 2 is a schematic cross-sectional view of an electronic apparatus according to an embodiment.

The electronic apparatus (e.g., a light-emitting apparatus) of FIG. 2 includes a substrate 100, a thin-film transistor (TFT), a light-emitting device, and an encapsulation portion 300 that seals the light-emitting device.

The substrate 100 may be a flexible substrate, a glass substrate, or a metal substrate. A buffer layer 210 may be arranged on the substrate 100. The buffer layer 210 may prevent penetration of impurities through the substrate 100 and may provide a flat surface on the substrate 100.

A TFT may be arranged on the buffer layer 210. The TFT may include an active layer 220, a gate electrode 240, a source electrode 260, and a drain electrode 270.

The active layer 220 may include an inorganic semiconductor, such as silicon or polysilicon, an organic semiconductor, or an oxide semiconductor, and may include a source region, a drain region, and a channel region.

A gate insulating film 230 for insulating the active layer 220 from the gate electrode 240 may be arranged on the active layer 220, and the gate electrode 240 may be arranged on the gate insulating film 230.

An interlayer insulating film 250 may be arranged on the gate electrode 240. The interlayer insulating film 250 may be arranged between the gate electrode 240 and the source electrode 260 to insulate the gate electrode 240 from the source electrode 260 and between the gate electrode 240 and the drain electrode 270 to insulate the gate electrode 240 from the drain electrode 270.

The source electrode 260 and the drain electrode 270 may be arranged on the interlayer insulating film 250. The interlayer insulating film 250 and the gate insulating film 230 may be formed to expose a source region and a drain region of the active layer 220, and the source electrode 260 and the drain electrode 270 may respectively contact the exposed portions of the source region and the drain region of the active layer 220.

The TFT may be electrically connected to a light-emitting device to drive the light-emitting device, and may be covered and protected by a passivation layer 280. The passivation layer 280 may include an inorganic insulating film, an organic insulating film, or any combination thereof. A light-emitting device may be provided on the passivation layer 280. The light-emitting device may include a first electrode 110, an interlayer 130, and a second electrode 150.

The first electrode 110 may be arranged on the passivation layer 280. The passivation layer 280 may not completely cover the drain electrode 270 and may expose a portion of the drain electrode 270. The first electrode 110 may be electrically connected to the exposed portion of the drain electrode 270.

A pixel defining layer 290 including an insulating material may be arranged on the first electrode 110. The pixel defining layer 290 may expose a selected region of the first electrode 110, and an interlayer 130 may be formed in the exposed region of the first electrode 110. The pixel defining layer 290 may be a polyimide-based organic film or a polyacrylic-based organic film. Although not shown in FIG. 2, at least some layers of the interlayer 130 may extend beyond the upper portion of the pixel defining layer 290 to be provided in the form of a common layer.

The second electrode 150 may be arranged on the interlayer 130, and a second capping layer 170 may be further included on the second electrode 150. The second capping layer 170 may be formed to cover the second electrode 150.

The encapsulation portion 300 may be arranged on the second capping layer 170. The encapsulation portion 300 may be arranged on a light-emitting device to protect the light-emitting device from moisture and/or oxygen. The encapsulation portion 300 may include: an inorganic film including silicon nitride (SiNₓ), silicon oxide (SiOₓ), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, or the like), an epoxy-based resin (for example, aliphatic glycidyl ether (AGE), or the like), or any combination thereof; or any combination of the inorganic films and the organic films.

FIG. 3 is a schematic cross-sectional view of the electronic apparatus according to another embodiment.

The electronic apparatus (e.g., a light-emitting apparatus) of FIG. 3 may differ from the electronic apparatus of FIG. 2, at least in that a light-shielding pattern 500 and a functional region 400 are further included on the encapsulation portion 300. The functional region 400 may be a color filter area, a color conversion area, or a combination of the color filter area and the color conversion area. In an embodiment, the light-emitting device included in the electronic apparatus of FIG. 3 may be a tandem light-emitting device.

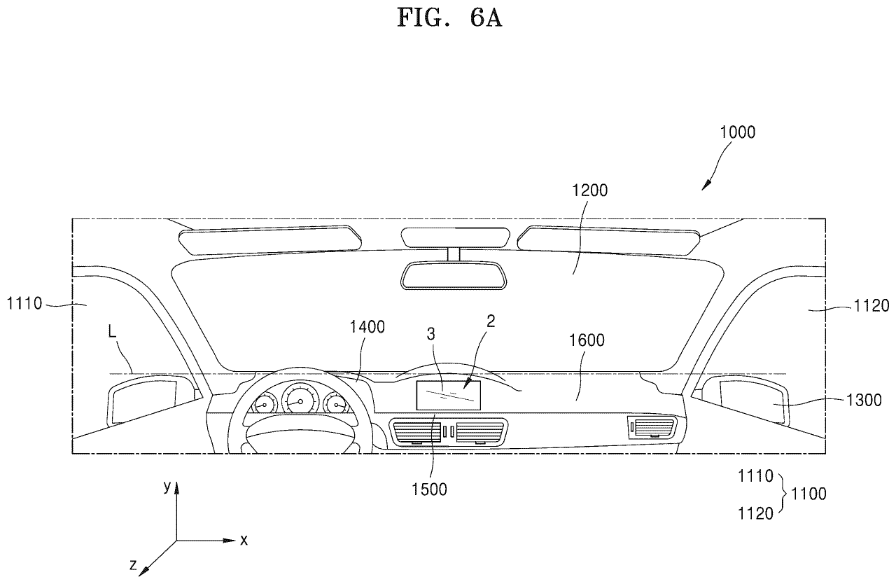

### [Description of FIG. 4]

FIG. 4 is a schematic perspective view of an electronic device 1 including a light-emitting device according to an embodiment.

The electronic device 1, which may be a device or apparatus that displays a moving image or still image, may be not only a portable electronic device, such as a mobile phone, a smartphone, a tablet personal computer (PC), a mobile communication terminal, an electronic notebook, an electronic book, a portable multimedia player (PMP), a navigation device, or an ultra-mobile PC (UMPC), but may also be various products, such as a television (TV), a laptop computer, a monitor, an advertisement board, or an Internet of things (IoT). The electronic device 1 may be such a product as above or a part thereof.

The electronic device 1 may be a wearable device, such as a smart watch, a watch phone, a glasses-type display, or a head mounted display (HMD), or a part of the wearable device. However, embodiments are not limited thereto.

For example, the electronic device 1 may be a dashboard of a vehicle, a center fascia of a vehicle, a center information display arranged on a dashboard of a vehicle, a room mirror display replacing a side mirror of a vehicle, an entertainment display for a rear seat of a vehicle or a display arranged on the back of a front seat, a head up display (HUD) installed in the front of a vehicle or projected on a front window glass, or a computer generated hologram augmented reality head up display (CGH AR HUD). FIG. 4 illustrates an embodiment in which the electronic device 1 is a smartphone, for convenience of explanation.

The electronic device 1 may include a display area DA and a non-display area NDA outside the display area DA. A display device may implement an image through a two-dimensional array of pixels that are arranged in the display area DA.

The non-display area NDA is an area that does not display an image, and may surround the display area DA. A driver for providing electrical signals or power to display devices arranged on the display area DA may be arranged in the non-display area NDA. A pad, which is an area to which an electronic element or a printing circuit board may be electrically connected, may be arranged in the non-display area NDA.

In the electronic device 1, a length in an x-axis direction and a length in a y-axis direction may be different from each other. In an embodiment, as shown in FIG. 4, the length in the x-axis direction may be shorter than the length in the y-axis direction. In embodiments, the length in the x-axis direction may be the same as the length in the y-axis direction. In still other embodiments, the length in the x-axis direction may be longer than the length in the y-axis direction.

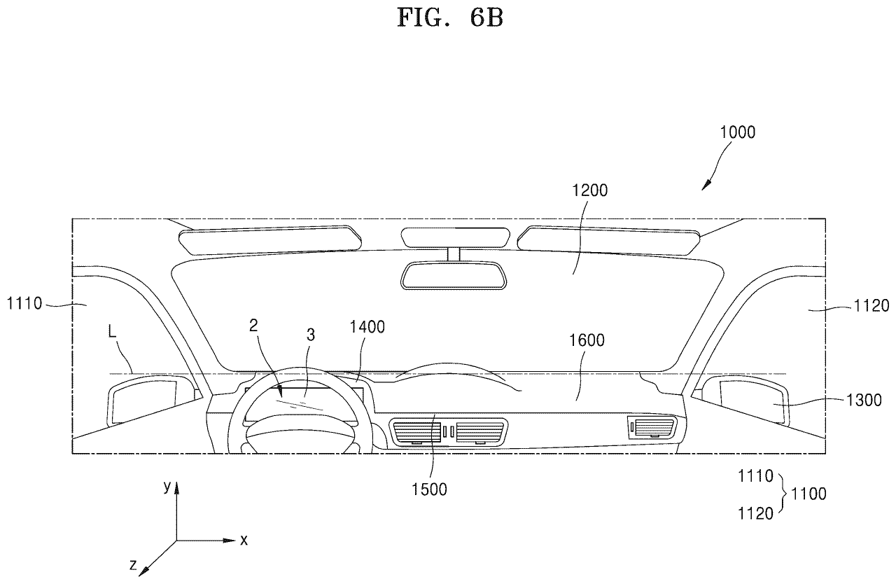

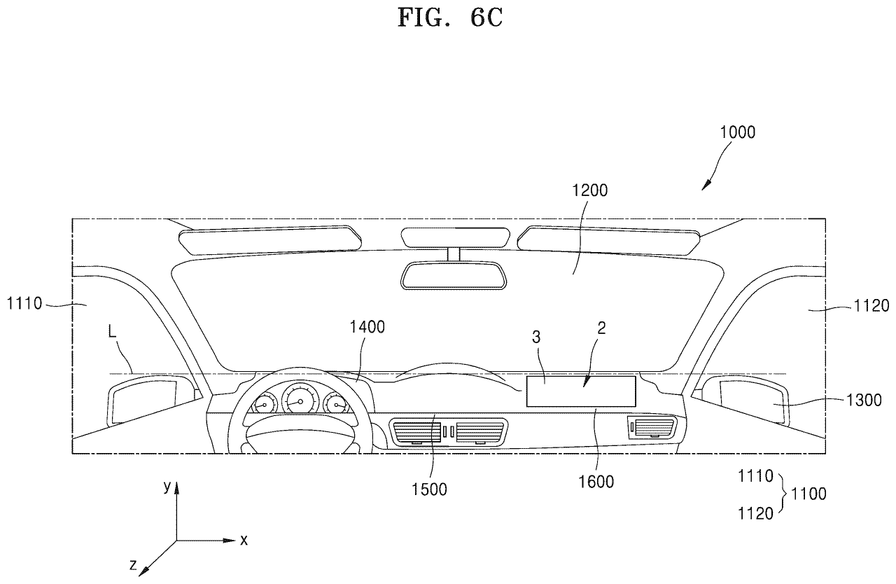

### [Descriptions of FIGS. 5 and 6A to 6C]

FIG. 5 is a schematic perspective view of an exterior of a vehicle 1000 as an electronic device including a light-emitting device according to an embodiment.

FIGS. 6A to 6C are each a schematic diagram of an interior of the vehicle 1000 according to embodiments.

Referring to FIGS. 5, 6A, 6B, and 6C, the vehicle 1000 may refer to various apparatuses for moving a subject to be transported, such as a person, an object, or an animal, from a departure point to a destination point. Examples of the vehicle 1000 may include a vehicle traveling on a road or track, a vessel moving over a sea or river, an airplane flying in the sky using the action of air, and the like.

The vehicle 1000 may travel on a road or a track. The vehicle 1000 may move in a given direction according to the rotation of at least one wheel. Examples of the vehicle 1000 may include a three-wheeled or four-wheeled vehicle, a construction machine, a two-wheeled vehicle, a prime mover, a bicycle, and a train running on a track.

The vehicle 1000 may include a body having an interior and an exterior, and a chassis that is a portion excluding the body in which mechanical apparatuses necessary for driving are installed. The exterior of the body may include a front panel, a bonnet, a roof panel, a rear panel, a trunk, a pillar provided at a boundary between doors, and the like. The chassis of the vehicle 1000 may include a power generating device, a power transmitting device, a driving device, a steering device, a braking device, a suspension device, a transmission device, a fuel device, front, rear, left, and right wheels, and the like.

The vehicle 1000 may include a side window glass 1100, a front window glass 1200, a side mirror 1300, a cluster 1400, a center fascia 1500, a passenger seat dashboard 1600, and a display device 2.

The side window glass 1100 and the front window glass 1200 may be partitioned by a pillar arranged between the side window glass 1100 and the front window glass 1200.

The side window glass 1100 may be installed on the side of the vehicle 1000. In an embodiment, the side window glass 1100 may be installed in a door of the vehicle 1000. Multiple side window glasses 1100 may be provided and may face each other. In an embodiment, the side window glass 1100 may include a first side window glass 1110 and a second side window glass 1120. In an embodiment, the first side window glass 1110 may be arranged adjacent to the cluster 1400, and the second side window glass 1120 may be arranged adjacent to the passenger seat dashboard 1600.

In an embodiment, the side window glasses 1100 may be spaced apart from each other in an x-direction or in a −x-direction. For example, the first side window glass 1110 and the second side window glass 1120 may be spaced apart from each other in the x direction or in the −x direction. An imaginary straight line L connecting the side window glasses 1100 may extend in the x-direction or the −x-direction. For example, an imaginary straight line L connecting the first side window glass 1110 and the second side window glass 1120 to each other may extend in the x direction or in the −x direction.

The front window glass 1200 may be installed on the front of the vehicle 1000. The front window glass 1200 may be between the side window glasses 1100 facing each other.

The side mirror 1300 may provide a rear view of the vehicle 1000. The side mirror 1300 may be installed on the exterior of the vehicle body. In an embodiment, multiple side mirrors 1300 may be provided. One of side mirrors 1300 may be arranged outside the first side window glass 1110. Another of the side mirrors 1300 may be arranged outside the second side window glass 1120.

The cluster 1400 may be arranged in front of a steering wheel. The cluster 1400 may include a tachometer, a speedometer, a coolant thermometer, a fuel gauge, a turn signal indicator, a high beam indicator, a warning light, a seat belt warning light, an odometer, a driving record system, an automatic shift selector indicator light, a door open warning light, an engine oil warning light, and/or a low fuel warning light.

The center fascia 1500 may include a control panel on which buttons for adjusting an audio device, an air conditioning device, and a seat heater are disposed. The center fascia 1500 may be arranged on a side of the cluster 1400.

A passenger seat dashboard 1600 may be spaced apart from the cluster 1400 with the center fascia 1500 arranged therebetween. In an embodiment, the cluster 1400 may be arranged to correspond to a driver seat (not shown), and the passenger seat dashboard 1600 may be disposed to correspond to a passenger seat (not shown). In an embodiment, the cluster 1400 may be adjacent to the first side window glass 1110, and the passenger seat dashboard 1600 may be adjacent to the second side window glass 1120.

In an embodiment, the display device 2 may include a display panel 3, and the display panel 3 may display an image. The display device 2 may be arranged inside the vehicle 1000. In an embodiment, the display device 2 may be between the side window glasses 1100 facing each other. The display device 2 may be arranged in at least one of the cluster 1400, the center fascia 1500, and the passenger seat dashboard 1600.

The display device 2 may include an organic light-emitting display device, an inorganic EL display device, a quantum dot display device, or the like. Hereinafter, as the display device 2 according to an embodiment of the disclosure, an organic light-emitting display device display including the light-emitting device according to the disclosure will be described as an example, but various types of display devices as described herein may be used as embodiments of the disclosure.

Referring to FIG. 6A, the display device 2 may be arranged in the center fascia 1500. In an embodiment, the display device 2 may display navigation information. In an embodiment, the display device 2 may display information regarding audio settings, video settings, or vehicle settings.

Referring to FIG. 6B, the display device 2 may be arranged in the cluster 1400. When the display device 2 is arranged in the cluster 1400, the cluster 1400 may display driving information and the like through the display device 2. For example, the cluster 1400 may digitally implement driving information. The cluster 1400 may digitally display vehicle information and driving information as images. For example, a needle and a gauge of a tachometer and various warning lights or icons may be displayed by a digital signal.

Referring to FIG. 6C, the display device 2 may be arranged in the passenger seat dashboard 1600. The display device 2 may be embedded in the passenger seat dashboard 1600 or arranged on the passenger seat dashboard 1600. In an embodiment, the display device 2 arranged on the passenger seat dashboard 1600 seat may display an image related to information displayed on the cluster 1400 and/or information displayed on the center fascia 1500. In embodiments, the display device 2 arranged on the passenger seat dashboard 1600 may display information different from information that is displayed on the cluster 1400 and/or different from information displayed on the center fascia 1500.

### [Manufacturing Method]

The layers constituting the hole transport region, the emission layer, and the layers constituting the electron transport region may be formed in a selected region by using various methods such as vacuum deposition, spin coating, casting, Langmuir-Blodgett (LB) deposition, ink-jet printing, laser-printing, laser-induced thermal imaging, and the like.

When the layers constituting the hole transport region, the emission layer, and the layers constituting the electron transport region are formed by vacuum deposition, the deposition may be performed at a deposition temperature in a range of about 100° C. to about 500° C., at a vacuum degree in a range of about 10⁻⁸ torr to about 10⁻³ torr, and at a deposition speed in a range of about 0.01 Å/sec to about 100 Å/sec, depending on a material to be included in a layer to be formed and the structure of a layer to be formed.

### Definitions of Terms

The term “C₅-C₆₀ carbocyclic group” as used herein may be a cyclic group consisting of carbon atoms as the only ring-forming atoms and having three to sixty carbon atoms, and the term “C₁-C₆₀ heterocyclic group” as used herein may be a cyclic group that has one to sixty carbon atoms and further has, in addition to a carbon atom, at least one heteroatom as a ring-forming atom. The C₅-C₆₀ carbocyclic group and the C₁-C₆₀ heterocyclic group may each be a monocyclic group consisting of one ring or a polycyclic group in which two or more rings are condensed with each other. For example, a C₁-C₆₀ heterocyclic group may have from 3 to 61 ring-forming atoms.

The term “cyclic group” as used herein may be a C₅-C₆₀ carbocyclic group or a C₁-C₆₀ heterocyclic group.

The term “π electron-rich C₃-C₆₀ cyclic group” as used herein may be a cyclic group that has 3 to 60 carbon atoms and may not include *—N═*′ as a ring-forming moiety, and the term “π electron-deficient nitrogen-containing C₁-C₆₀ heterocyclic group” as used herein may be a heterocyclic group that has 1 to 60 carbon atoms and may include *—N═*′ as a ring-forming moiety.

In embodiments, a C₃-C₆₀ carbocyclic group may be a T₁ group or a group in which two or more of T₁ groups are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),
a C₁-C₆₀ heterocyclic group may be a T2 group, a group in which two or more T2 groups are condensed with each other, or a group in which at least one T2 group and at least one T1 group are condensed with each other (e.g., a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),
a π electron-rich C₃-C₆₀ cyclic group may be a T1 group, a group in which two or more T1 groups are condensed with each other, a T3 Group, a group in which two or more T3 groups are condensed with each other, or a group in which at least one T3 group and at least one T1 group are condensed with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, or the like),
the π electron-deficient nitrogen-containing C₁-C₆₀ heterocyclic group may be a T4 group, a group in which two or more T4 groups are condensed with each other, a group in which at least one T4 group and at least one T1 group are condensed with each other, a group in which at least one T4 group and at least one T3 group are condensed with each other, or a group in which at least one T4 group, at least one T1 group, and at least one T3 group are condensed with one another (e.g., a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.), wherein
the T1 group may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or bicyclo[2.2.1]heptane) group, a norbornene group, a bicyclo[1.1.1]pentane group, a bicyclo[2.1.1]hexane group, a bicyclo[2.2.2]octane group, or a benzene group,
the T2 group may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,
the T3 group may be a furan group, a thiophene group, a 1H-pyrrole group, a silole group, or a borole group, and
the T4 group may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “cyclic group”, “C₃-C₆₀ carbocyclic group”, “C₁-C₆₀ heterocyclic group”, “π electron-rich C₃-C₆₀ cyclic group”, and “IT electron-deficient nitrogen-containing C₁-C₆₀ heterocyclic group” as used herein may each be a group condensed to any cyclic group, a monovalent group, or a polyvalent group (e.g., a divalent group, a trivalent group, a tetravalent group, etc.) according to the structure of a formula for which the corresponding term is used. For example, a “benzene group” may be a benzo group, a phenyl group, a phenylene group, or the like, which may be readily understood by one of ordinary skill in the art according to the structure of a formula including the “benzene group.”

Examples of a monovalent C₅-C₆₀ carbocyclic group or a monovalent C₁-C₆₀ heterocyclic group may include a C₃-C₁₀ cycloalkyl group, a C₁-C₁₀ heterocycloalkyl group, a C₅-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group. Examples of a divalent C₃-C₆₀ carbocyclic group or a divalent C₁-C₆₀ heterocyclic group may include a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a divalent non-aromatic condensed heteropolycyclic group.

The term “C₁-C₆₀ alkyl group” as used herein may be a linear or branched aliphatic hydrocarbon monovalent group that has one to sixty carbon atoms, and examples thereof may include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group. The term “C₁-C₆₀ alkylene group” as used herein may be a divalent group having a same structure as the C₁-C₆₀ alkyl group.

The term “C₂-C₆₀ alkenyl group” as used herein may be a monovalent hydrocarbon group having at least one carbon-carbon double bond in the middle or at a terminus of a C₂-C₆₀ alkyl group, and examples thereof may include an ethenyl group, a propenyl group, a butenyl group, and the like. The term “C₂-C₆₀ alkenylene group” as used herein may be a divalent group having a same structure as the C₂-C₆₀ alkenyl group.

The term “C₂-C₆₀ alkynyl group” as used herein may be a monovalent hydrocarbon group having at least one carbon-carbon triple bond in the middle or at a terminus of a C₂-C₆₀ alkyl group, and examples thereof may be an ethynyl group, a propynyl group, and the like. The term “C₂-C₆₀ alkynylene group” as used herein may be a divalent group having the same structure as the C₂-C₆₀ alkynyl group.

The term “C₁-C₆₀ alkoxy group” as used herein may be to a monovalent group represented by —O(A₁₀₁) (wherein A₁₀₁ may be a C₁-C₆₀ alkyl group), and examples thereof may include a methoxy group, an ethoxy group, an isopropyloxy group, and the like.

The term “C₃-C₁₀ cycloalkyl group” as used herein may be a monovalent saturated hydrocarbon cyclic group having 3 to 10 carbon atoms, and examples thereof may include a cyclopropyl group, a cyclobutyl group, a cyclopentyl group, a cyclohexyl group, a cycloheptyl group, a cyclooctyl group, an adamantanyl group, a norbornanyl group (or bicyclo[2.2.1]heptyl group), a bicyclo[1.1.1]pentyl group, a bicyclo[2.1.1]hexyl group, a bicyclo[2.2.2]octyl group, and the like. The term “C₃-C₁₀ cycloalkylene group” as used herein may be a divalent group having a same structure as the C₃-C₁₀ cycloalkyl group.

The term “C₁-C₁₀ heterocycloalkyl group” as used herein may be a monovalent cyclic group having 1 to 10 carbon atoms, further including, in addition to carbon atoms, at least one heteroatom as a ring-forming atom, and examples thereof may include a 1,2,3,4-oxatriazolidinyl group, a tetrahydrofuranyl group, a tetrahydrothiophenyl group, and the like. The term “C₁-C₁₀ heterocycloalkylene group” as used herein may be a divalent group having a same structure as the C₁-C₁₀ heterocycloalkyl group.

The term “C₃-C₁₀ cycloalkenyl group” as used herein may be a monovalent cyclic group that has three to ten carbon atoms and at least one carbon-carbon double bond in the cyclic structure thereof and no aromaticity, and examples thereof may include a cyclopentenyl group, a cyclohexenyl group, a cycloheptenyl group, and the like. The term “C₃-C₁₀ cycloalkenylene group” as used herein may be a divalent group having a same structure as the C₃-C₁₀ cycloalkenyl group.

The term “C₁-C₁₀ heterocycloalkenyl group” as used herein may be a monovalent cyclic group of 1 to 10 carbon atoms, further including, in addition to carbon atoms, at least one heteroatom as a ring-forming atom, and having at least one carbon-carbon double bond in the cyclic structure thereof. Examples of a C₁-C₁₀ heterocycloalkenyl group may include a 4,5-dihydro-1,2,3,4-oxatriazolyl group, a 2,3-dihydrofuranyl group, a 2,3-dihydrothiophenyl group, and the like. The term “C₁-C₁₀ heterocycloalkenylene group” as used herein may be a divalent group having a same structure as the C₁-C₁₀ heterocycloalkenyl group.

The term “C₆-C₆₀ aryl group” as used herein may be a monovalent group having a carbocyclic aromatic system of 6 to 60 carbon atoms, and the term “C₆-C₆₀ arylene group” as used herein may be a divalent group having a carbocyclic aromatic system of 6 to 60 carbon atoms. Examples of a C₆-C₆₀ aryl group may include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, an ovalenyl group, and the like. When the C₆-C₆₀ aryl group and the C₆-C₆₀ arylene group each include two or more rings, the respective rings may be condensed with each other.

The term “C₁-C₆₀ heteroaryl group” as used herein may be a monovalent group having a heterocyclic aromatic system of 1 to 60 carbon atoms, further including, in addition to carbon atoms, at least one heteroatom as a ring-forming atom. The term “C₁-C₆₀ heteroarylene group” as used herein may be a divalent group having a heterocyclic aromatic system of 1 to 60 carbon atoms, further including, in addition to carbon atoms, at least one heteroatom as a ring-forming atom. Examples of a C₁-C₆₀ heteroaryl group may include a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, and a naphthyridinyl group. When the C₁-C₆₀ heteroaryl group and the C₁-C₆₀ heteroarylene group each include two or more rings, the respective rings may be condensed with each other.

The term “monovalent non-aromatic condensed polycyclic group” as used herein may be a monovalent group (for example, having 8 to 60 carbon atoms) having two or more rings condensed to each other, only carbon atoms as ring-forming atoms, and no aromaticity in its molecular structure as a whole. Examples of a monovalent non-aromatic condensed polycyclic group may include an indenyl group, a fluorenyl group, a spiro-bifluorenyl group, a benzofluorenyl group, an indenophenanthrenyl group, an indeno anthracenyl group, and the like. The term “divalent non-aromatic condensed polycyclic group” as used herein may be a divalent group having a same structure as the monovalent non-aromatic condensed polycyclic group described above.

The term “monovalent non-aromatic condensed heteropolycyclic group” as used herein may be a monovalent group (for example, having 1 to 60 carbon atoms) having two or more rings condensed to each other, further including, in addition to carbon atoms, at least one heteroatom as a ring-forming atom, and having non-aromaticity in its molecular structure as a whole. Examples of a monovalent non-aromatic condensed heteropolycyclic group may include a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indeno carbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, and a benzothienodibenzothiophenyl group. The term “divalent non-aromatic condensed heteropolycyclic group” as used herein may be a divalent group having a same structure as the monovalent non-aromatic condensed heteropolycyclic group described above.

The term “C₆-C₆₀ aryloxy group” as used herein may be a group represented by —O(A₁₀₂) (wherein A₁₀₂ may be a C₆-C₆₀ aryl group), and the term “C₆-C₆₀ arylthio group” as used herein may be a group represented by —S(A₁₀₃) (wherein A₁₀₃ may be a C₆-C₆₀ aryl group).

The term “C₇-C₆₀ arylalkyl group” as used herein may be a group represented by —(A₁₀₄)(A₁₀₅) (wherein A₁₀₄ may be a C₁-C₅₄ alkylene group, and A₁₀₅ may be a C₆-C₅₉ aryl group), and the term “C₂-C₆₀ heteroarylalkyl group” as used herein may be a group represented by —(A₁₀₆)(A₁₀₇) (wherein A₁₀₆ may be a C₁-C₅₉ alkylene group, and A₁₀₇ may be a C₁-C₅₉ heteroaryl group).

In the specification, the group “R₁₀ₐ” may be:
deuterium (—D), —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₅-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

In the specification, the groups Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group, a C₂-C₆₀ alkynyl group; a C₁-C₆₀ alkoxy group; or a C₅-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

The term “heteroatom” as used herein may be any atom other than a carbon atom or a hydrogen atom. Examples of a heteroatom may include O, S, N, P, Si, B, Ge, Se, and any combination thereof.

The term “third-row transition metal” as used herein may include Hf, Ta, W, Re, Os, Ir, Pt, Au, or the like.

In the specification, “Ph” refers to a phenyl group, “Me” refers to a methyl group, “Et” refers to an ethyl group, “ter-Bu” or “But” each refers to a tert-butyl group, and “OMe” refers to a methoxy group.

The term “biphenyl group” as used herein may be a “phenyl group substituted with a phenyl group.” For example, the “biphenyl group” may be a phenyl group substituted with a C₆-C₆₀ aryl group.

The term “terphenyl group” as used herein may be a “phenyl group substituted with a biphenyl group.” For example, the “terphenyl group” may be a substituted phenyl group having, as a substituent, a C₆-C₆₀ aryl group substituted with a C₆-C₆₀ aryl group.

The symbols * and *′ as used herein, unless defined otherwise, each refer to a binding site to a neighboring atom in a corresponding formula or moiety.

In the specification, the terms “x-axis”, “y-axis”, and “z-axis” are not limited to three axes in an orthogonal coordinate system (e.g., a Cartesian coordinate system), and may be interpreted in a broader sense than the aforementioned axes that are orthogonal to each other. For example, the x-axis, y-axis, and z-axis may describe axes that are orthogonal to each other, or may describe axes that are in different directions that are not orthogonal to each other.

Hereinafter, compounds according to embodiments and light-emitting devices according to embodiments will be described in detail with reference to the following Synthesis Examples and Examples. The wording “B was used instead of A” used in describing Synthesis Examples means that an identical molar equivalent of B was used in place of A.

### Synthesis Example

### Synthesis Example 1: Synthesis of Compound 1

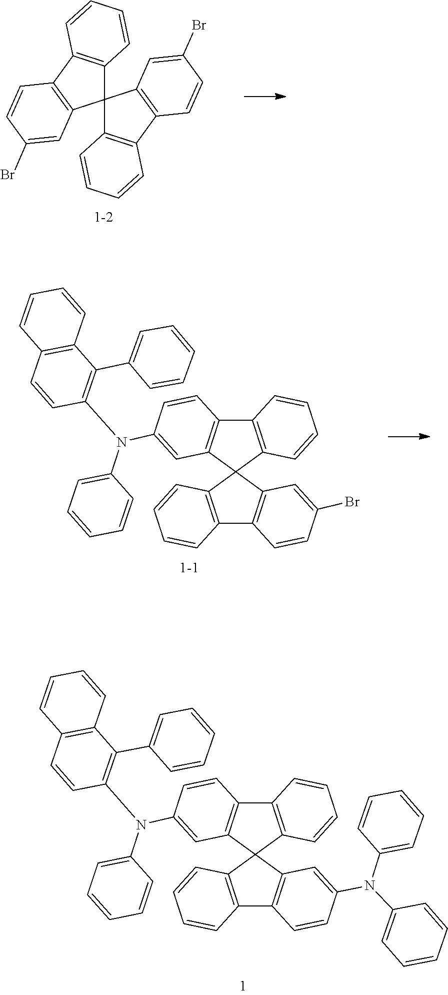 [SMILES: Brc1ccc2c(c1)C1(c3ccccc3-2)c2ccccc2-c2ccc(Br)cc21 ; Brc1ccc2c(c1)C1(c3ccccc3-2)c2ccccc2-c2ccc(N(c3ccccc3)c3ccc4ccccc4c3-c3ccccc3)cc21 ; c1ccc(-c2c(N(c3ccccc3)c3ccc4c(c3)C3(c5ccccc5-c5ccc(N(c6ccccc6)c6ccccc6)cc53)c3ccccc3-4)ccc3ccccc23)cc1]

(Synthesis of Compound 1-1) 2,2′-dibromo-9,9′-spirobi [fluorene](26 mmol, 1.3 equiv.), N,1-diphenylnaphthalen-2-amine (20 mmol, 1 equiv.), Pd₂(dba)₃ (1 mmol, 0.05 equiv.), t-BuONa (40 mmol, 2 equiv.), BINAP(2 mmol, 0.1 equiv.), and 300 ml of toluene (Tol.) were added to a one-neck round flask and stirred at 110° C. for an hour.

After the reaction was completed, the reaction product was worked up with ether/H₂O and purified by column chromatography using dichloromethane/hexane (1/5) to obtain 14 mmol of Compound 1-1 (yield=70%).

(Synthesis of Compound 1) Compound 1-1 (12 mmol, 1.2 equiv.), diphenylamine (10 mmol, 1 equiv.), Pd₂(dba)₃ (0.5 mol, 0.05 equiv.), t-BuONa (20 mmol, 2 equiv.), t-Bu₃P (0.1 mmol, 0.1 equiv.), and 150 ml of toluene (Tol.) were added to a one-neck round flask and stirred at 110° C. for an hour.

After the reaction was completed, the reaction product was worked up with ether/H₂O and purified by column chromatography using dichloromethane/hexane (1/8) to obtain 8.0 mmol of Compound 1 (yield=80%).

### Synthesis Example 2: Synthesis of Compound 2

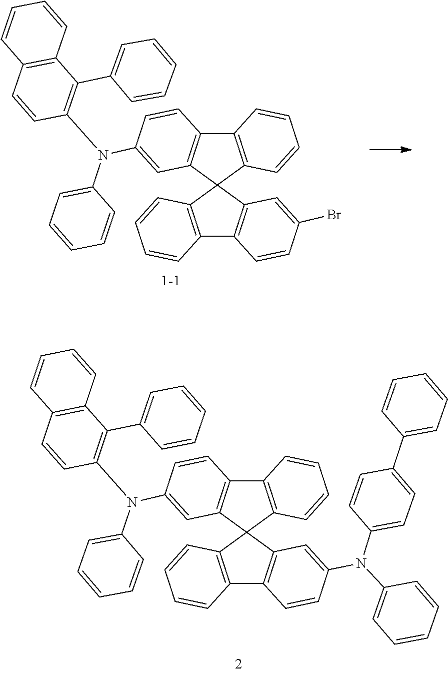 [SMILES: Brc1ccc2c(c1)C1(c3ccccc3-2)c2ccccc2-c2ccc(N(c3ccccc3)c3ccc4ccccc4c3-c3ccccc3)cc21 ; c1ccc(-c2ccc(N(c3ccccc3)c3ccc4c(c3)C3(c5ccccc5-4)c4ccccc4-c4ccc(N(c5ccccc5)c5ccc6ccccc6c5-c5ccccc5)cc43)cc2)cc1]

(Synthesis of Compound 2) Compound 1-1 (12 mmol, 1.2 equiv.), N-phenyl-[1,1′-biphenyl]-4-amine (10 mmol, 1 equiv.), Pd₂(dba)₃ (0.5 mol, 0.05 equiv.), t-BuONa (20 mmol, 2 equiv.), t-Bu₃P (0.1 mmol, 0.1 equiv.), and 150 ml of Tol. were added to a one-neck round flask and stirred at 110° C. for an hour.

After the reaction was completed, the reaction product was worked up with ether/H₂O and purified by column chromatography using dichloromethane/hexane (1/8) to obtain 7.8 mmol of Compound 2 (yield=78%).

### Synthesis Example 3: Synthesis of Compound 3

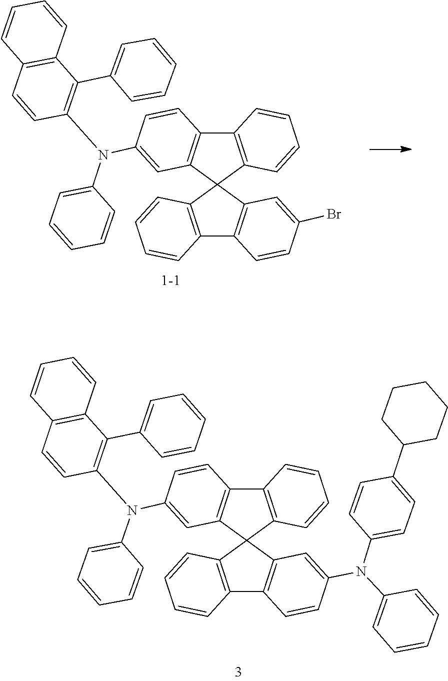 [SMILES: Brc1ccc2c(c1)C1(c3ccccc3-2)c2ccccc2-c2ccc(N(c3ccccc3)c3ccc4ccccc4c3-c3ccccc3)cc21 ; c1ccc(-c2c(N(c3ccccc3)c3ccc4c(c3)C3(c5ccccc5-c5ccc(N(c6ccccc6)c6ccc(C7CCCCC7)cc6)cc53)c3ccccc3-4)ccc3ccccc23)cc1]

(Synthesis of Compound 3) Compound 1-1 (12 mmol, 1.2 equiv.), 4-cyclohexyl-N-phenylaniline (10 mmol, 1 equiv.), Pd₂(dba)₃ (0.5 mol, 0.05 equiv.), t-BuONa (20 mmol, 2 equiv.), t-Bu₃P (0.1 mmol, 0.1 equiv.), and 150 ml of Tol. were added to a one-neck round flask and stirred at 110° C. for an hour.

After the reaction was completed, the reaction product was worked up with ether/H₂O and purified by column chromatography using dichloromethane/hexane (1/8) to obtain 7.9 mmol of Compound 3 (yield=79%).

### Synthesis Example 4: Synthesis of Compound 5

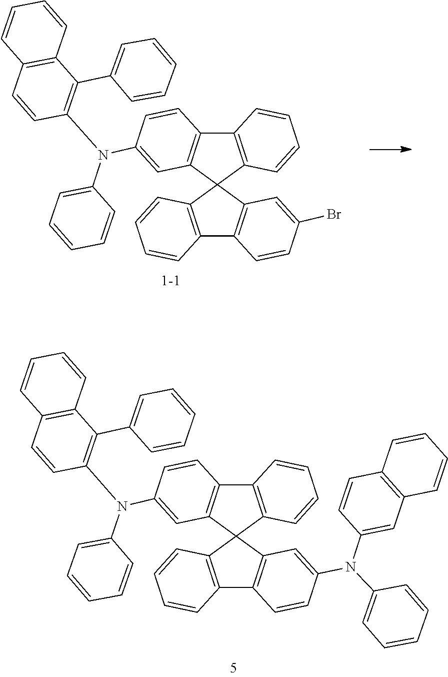 [SMILES: Brc1ccc2c(c1)C1(c3ccccc3-2)c2ccccc2-c2ccc(N(c3ccccc3)c3ccc4ccccc4c3-c3ccccc3)cc21 ; c1ccc(-c2c(N(c3ccccc3)c3ccc4c(c3)C3(c5ccccc5-c5ccc(N(c6ccccc6)c6ccc7ccccc7c6)cc53)c3ccccc3-4)ccc3ccccc23)cc1]

(Synthesis of Compound 5) Compound 1-1 (12 mmol, 1.2 equiv.), N-phenylnaphthalen-2-amine (10 mmol, 1 equiv.), Pd₂(dba)₃ (0.5 mol, 0.05 equiv.), t-BuONa (20 mmol, 2 equiv.), t-Bu₃P(0.1 mmol, 0.1 equiv.), and 150 ml of Tol. were added to a one-neck round flask and stirred at 110° C. for an hour.

After the reaction was completed, the reaction product was worked up with ether/H₂O and purified by column chromatography using dichloromethane/hexane (1/8) to obtain 7.5 mmol of Compound 5 (yield=75%).

### Synthesis Example 5: Synthesis of Compound 7

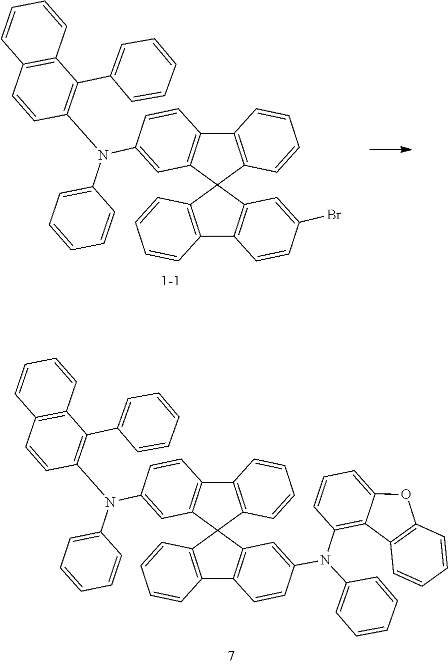 [SMILES: Brc1ccc2c(c1)C1(c3ccccc3-2)c2ccccc2-c2ccc(N(c3ccccc3)c3ccc4ccccc4c3-c3ccccc3)cc21 ; c1ccc(-c2c(N(c3ccccc3)c3ccc4c(c3)C3(c5ccccc5-4)c4ccccc4-c4ccc(N(c5ccccc5)c5cccc6oc7ccccc7c56)cc43)ccc3ccccc23)cc1]

(Synthesis of Compound 7) Compound 1-1 (12 mmol, 1.2 equiv.), N-phenyldibenzo[b,d]furan-1-amine (10 mmol, 1 equiv.), Pd₂(dba)₃ (0.5 mol, 0.05 equiv.), t-BuONa (20 mmol, 2 equiv.), t-Bu₃P (0.1 mmol, 0.1 equiv.), and 150 ml of Tol. were added to a one-neck round flask and stirred at 110° C. for an hour.

After the reaction was completed, the reaction product was worked up with ether/H₂O and purified by column chromatography using dichloromethane/hexane (1/8) to obtain 7.0 mmol of Compound 7 (yield=70%).

### Synthesis Example 6: Synthesis of Compound 9

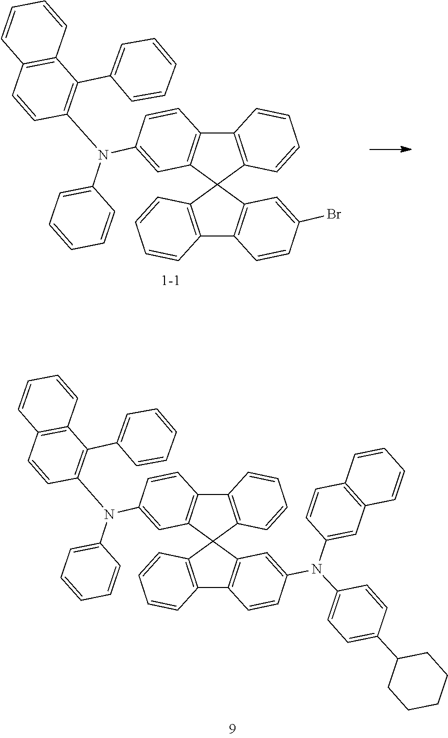 [SMILES: Brc1ccc2c(c1)C1(c3ccccc3-2)c2ccccc2-c2ccc(N(c3ccccc3)c3ccc4ccccc4c3-c3ccccc3)cc21 ; c1ccc(-c2c(N(c3ccccc3)c3ccc4c(c3)C3(c5ccccc5-c5ccc(N(c6ccc(C7CCCCC7)cc6)c6ccc7ccccc7c6)cc53)c3ccccc3-4)ccc3ccccc23)cc1]

(Synthesis of Compound 9) Compound 1-1 (12 mmol, 1.2 equiv.), N-(4-cyclohexylphenyl) naphthalen-2-amine (10 mmol, 1 equiv.), Pd₂(dba)₃ (0.5 mol, 0.05 equiv.), t-BuONa (20 mmol, 2 equiv.), t-Bu₃P (0.1 mmol, 0.1 equiv.), and 150 ml of Tol. were added to a one-neck round flask and stirred at 110° C. for an hour.

After the reaction was completed, the reaction product was worked up with ether/H₂O and purified by column chromatography using dichloromethane/hexane (1/8) to obtain 7.2 mmol of Compound 9 (yield=72%).

### Synthesis Example 7: Synthesis of Compound 12

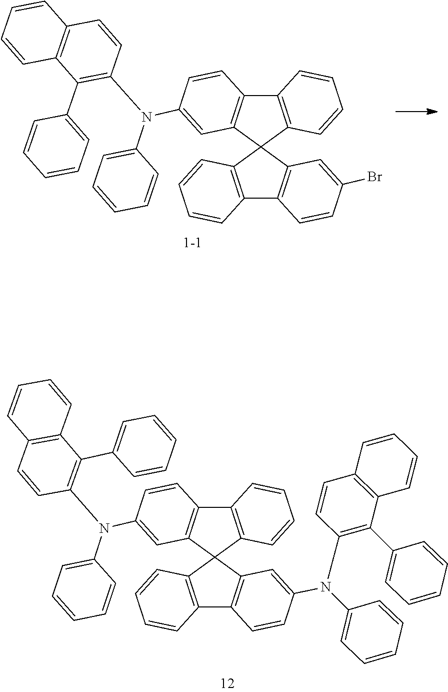 [SMILES: Brc1ccc2c(c1)C1(c3ccccc3-2)c2ccccc2-c2ccc(N(c3ccccc3)c3ccc4ccccc4c3-c3ccccc3)cc21 ; c1ccc(-c2c(N(c3ccccc3)c3ccc4c(c3)C3(c5ccccc5-4)c4ccccc4-c4ccc(N(c5ccccc5)c5ccc6ccccc6c5-c5ccccc5)cc43)ccc3ccccc23)cc1]

(Synthesis of Compound 12) Compound 1-1 (12 mmol, 1.2 equiv.), N,1-diphenylnaphthalen-2-amine (10 mmol, 1 equiv.), Pd₂(dba)₃ (0.5 mol, 0.05 equiv.), t-BuONa (20 mmol, 2 equiv.), t-Bu₃P (0.1 mmol, 0.1 equiv.), and 150 ml of Tol. were added to a one-neck round flask and stirred at 110° C. for an hour.

After the reaction was completed, the reaction product was worked up with ether/H₂O and purified by column chromatography using dichloromethane/hexane (1/8) to obtain 7.1 mmol of Compound 12 (yield=71%).

### Synthesis Example 8: Synthesis of Compound 16

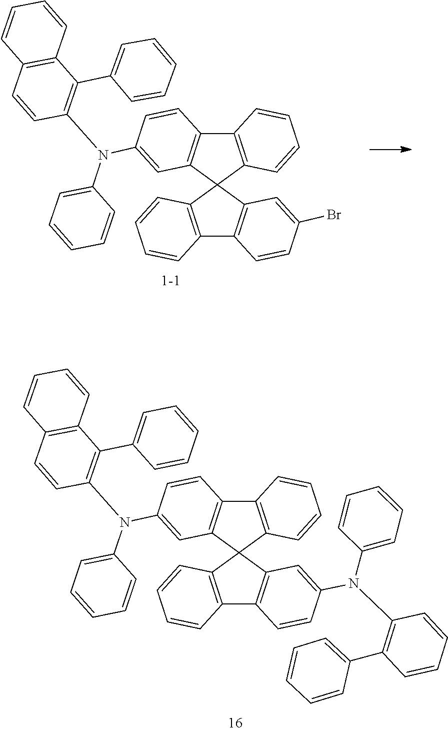 [SMILES: Brc1ccc2c(c1)C1(c3ccccc3-2)c2ccccc2-c2ccc(N(c3ccccc3)c3ccc4ccccc4c3-c3ccccc3)cc21 ; c1ccc(-c2ccccc2N(c2ccccc2)c2ccc3c(c2)C2(c4ccccc4-3)c3ccccc3-c3ccc(N(c4ccccc4)c4ccc5ccccc5c4-c4ccccc4)cc32)cc1]

(Synthesis of Compound 16) Compound 1-1 (12 mmol, 1.2 equiv.), N-phenyl-[1,1′-biphenyl]-2-amine (10 mmol, 1 equiv.), Pd₂(dba)₃ (0.5 mol, 0.05 equiv.), t-BuONa (20 mmol, 2 equiv.), t-Bu₃P (0.1 mmol, 0.1 equiv.), and 150 ml of Tol. were added to a one-neck round flask and stirred at 110° C. for an hour.

After the reaction was completed, the reaction product was worked up with ether/H₂O and purified by column chromatography using dichloromethane/hexane (1/8) to obtain 7.0 mmol of Compound 16 (yield=70%).

### Synthesis Example 9: Synthesis of Compound 33

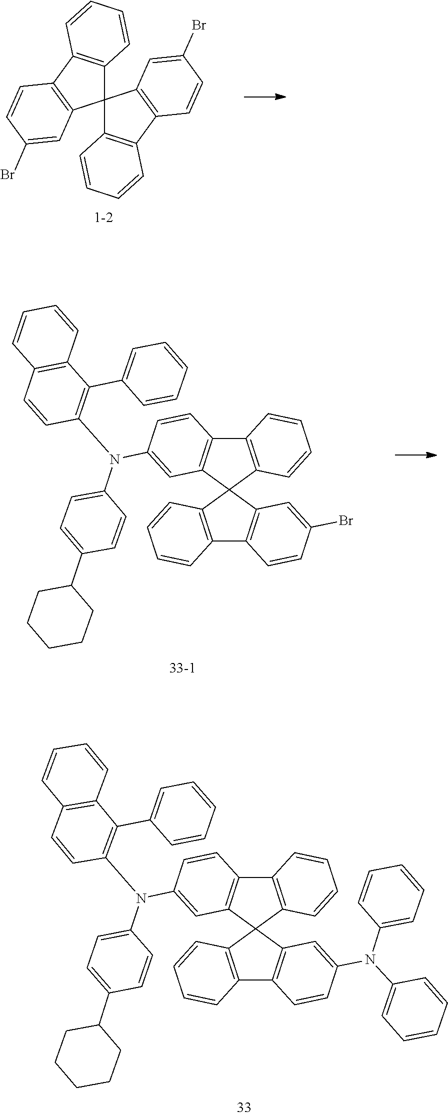 [SMILES: Brc1ccc2c(c1)C1(c3ccccc3-2)c2ccccc2-c2ccc(Br)cc21 ; Brc1ccc2c(c1)C1(c3ccccc3-2)c2ccccc2-c2ccc(N(c3ccc(C4CCCCC4)cc3)c3ccc4ccccc4c3-c3ccccc3)cc21 ; c1ccc(-c2c(N(c3ccc(C4CCCCC4)cc3)c3ccc4c(c3)C3(c5ccccc5-c5ccc(N(c6ccccc6)c6ccccc6)cc53)c3ccccc3-4)ccc3ccccc23)cc1]

(Synthesis of Compound 33-1) 2,2′-dibromo-9,9′-spirobi [fluorene] (26 mmol, 1.3 equiv.), N-(4-cyclohexylphenyl)-1-phenylnaphthalen-2-amine (20 mmol, 1 equiv.), Pd₂(dba)₃ (1 mmol, 0.05 equiv.), t-BuONa (40 mmol, 2 equiv.), BINAP(2 mmol, 0.1 equiv.), and 300 ml of Tol. were added to a one-neck round flask and stirred at 110° C. for an hour.

After the reaction was completed, the reaction product was worked up with ether/H₂O and purified by column chromatography using dichloromethane/hexane (1/5) to obtain 12 mmol of Compound 33-1 (yield=60%).

(Synthesis of Compound 33) Compound 33-1 (12 mmol, 1.2 equiv.), diphenylamine (10 mmol, 1 equiv.), Pd₂(dba)₃ (0.5 mol, 0.05 equiv.), t-BuONa (20 mmol, 2 equiv.), t-Bu₃P (0.1 mmol, 0.1 equiv.), and 150 ml of Tol. were added to a one-neck round flask and stirred at 110° C. for an hour.

After the reaction was completed, the reaction product was worked up with ether/H₂O and purified by column chromatography using dichloromethane/hexane (1/8) to obtain 7.8 mmol of Compound 33 (yield=78%).

### Synthesis Example 10: Synthesis of Compound 37

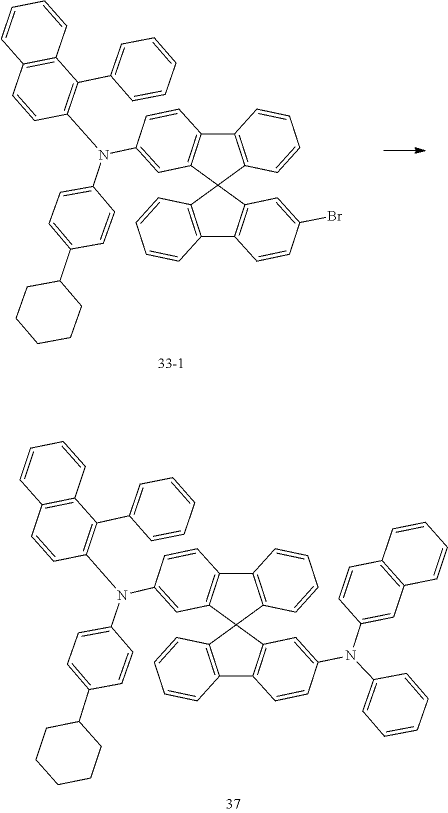 [SMILES: Brc1ccc2c(c1)C1(c3ccccc3-2)c2ccccc2-c2ccc(N(c3ccc(C4CCCCC4)cc3)c3ccc4ccccc4c3-c3ccccc3)cc21 ; c1ccc(-c2c(N(c3ccc(C4CCCCC4)cc3)c3ccc4c(c3)C3(c5ccccc5-c5ccc(N(c6ccccc6)c6ccc7ccccc7c6)cc53)c3ccccc3-4)ccc3ccccc23)cc1]

(Synthesis of Compound 37) Compound 33-1 (12 mmol, 1.2 equiv.), N-phenylnaphthalen-2-amine (10 mmol, 1 equiv.), Pd₂(dba)₃ (0.5 mol, 0.05 equiv.), t-BuONa (20 mmol, 2 equiv.), t-Bu₃P (0.1 mmol, 0.1 equiv.), and 150 ml of Tol. were added to a one-neck round flask and stirred at 110° C. for an hour.

After the reaction was completed, the reaction product was worked up with ether/H₂O and purified by column chromatography using dichloromethane/hexane (1/8) to obtain 7.6 mmol of Compound 37 (yield=76%).

### Synthesis Example 11: Synthesis of Compound 41

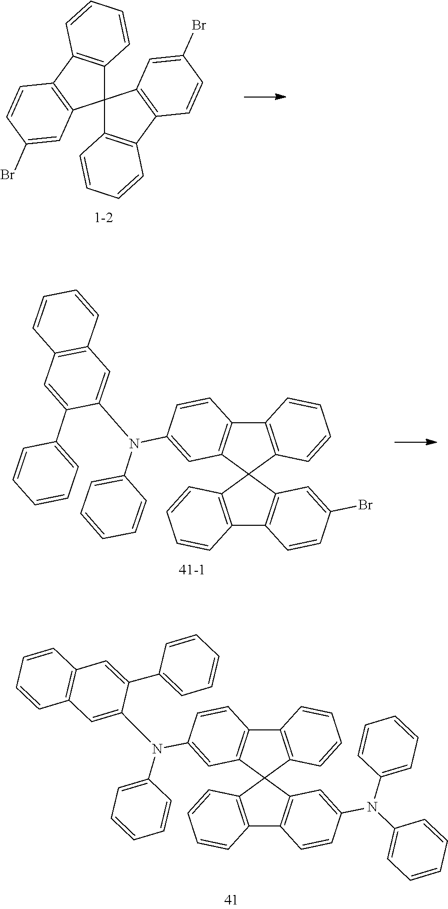 [SMILES: Brc1ccc2c(c1)C1(c3ccccc3-2)c2ccccc2-c2ccc(Br)cc21 ; Brc1ccc2c(c1)C1(c3ccccc3-2)c2ccccc2-c2ccc(N(c3ccccc3)c3cc4ccccc4cc3-c3ccccc3)cc21 ; c1ccc(-c2cc3ccccc3cc2N(c2ccccc2)c2ccc3c(c2)C2(c4ccccc4-c4ccc(N(c5ccccc5)c5ccccc5)cc42)c2ccccc2-3)cc1]

(Synthesis of Compound 41-1) 2,2′-dibromo-9,9′-spirobi [fluorene] (26 mmol, 1.3 equiv.), N,3-diphenylnaphthalen-2-amine (20 mmol, 1 equiv.), Pd₂(dba)₃ (1 mmol, 0.05 equiv.), t-BuONa (40 mmol, 2 equiv.), BINAP(2 mmol, 0.1 equiv.), and 300 ml of Tol. were added to a one-neck round flask and stirred at 110° C. for an hour.

After the reaction was completed, the reaction product was worked up with ether/H₂O and purified by column chromatography using dichloromethane/hexane (1/5) to obtain 14 mmol of Compound 41-1 (yield=70%).

(Synthesis of Compound 41) Compound 41-1 (12 mmol, 1.2 equiv.), diphenylamine (10 mmol, 1 equiv.), Pd₂(dba)₃ (0.5 mol, 0.05 equiv.), t-BuONa (20 mmol, 2 equiv.), t-Bu₃P (0.1 mmol, 0.1 equiv.), and 150 ml of Tol. were added to a one-neck round flask and stirred at 110° C. for an hour.

The reaction product was worked up with ether/H₂O and purified by column chromatography using dichloromethane/hexane (1/8) to obtain 8.2 mmol of Compound 41 (yield=82%).

### Synthesis Example 12: Synthesis of Compound 42

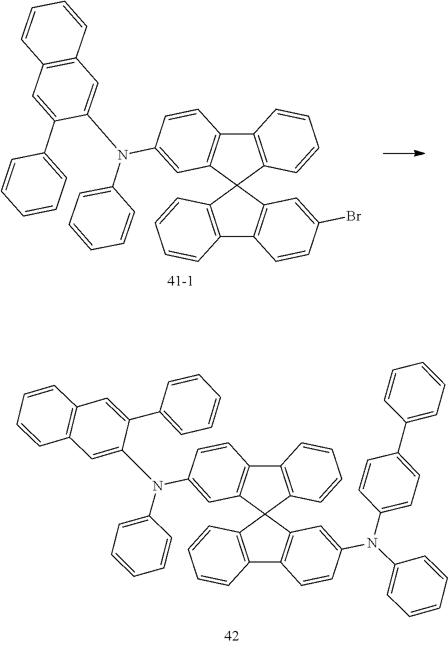 [SMILES: Brc1ccc2c(c1)C1(c3ccccc3-2)c2ccccc2-c2ccc(N(c3ccccc3)c3cc4ccccc4cc3-c3ccccc3)cc21 ; c1ccc(-c2ccc(N(c3ccccc3)c3ccc4c(c3)C3(c5ccccc5-4)c4ccccc4-c4ccc(N(c5ccccc5)c5cc6ccccc6cc5-c5ccccc5)cc43)cc2)cc1]

(Synthesis of Compound 42) Compound 41-1 (12 mmol, 1.2 equiv.), N-phenyl-[1,1′-biphenyl]-4-amine (10 mmol, 1 equiv.), Pd₂(dba)₃ (0.5 mol, 0.05 equiv.), t-BuONa (20 mmol, 2 equiv.), t-Bu₃P (0.1 mmol, 0.1 equiv.), and 150 ml of Tol. Were added to a one-neck round flask and stirred at 110° C. for an hour.

After the reaction was completed, the reaction product was worked up with ether/H₂O and purified by column chromatography using dichloromethane/hexane (1/8) to obtain 7.5 mmol of Compound 42 (yield=75%).

### Synthesis Example 13: Synthesis of Compound 43

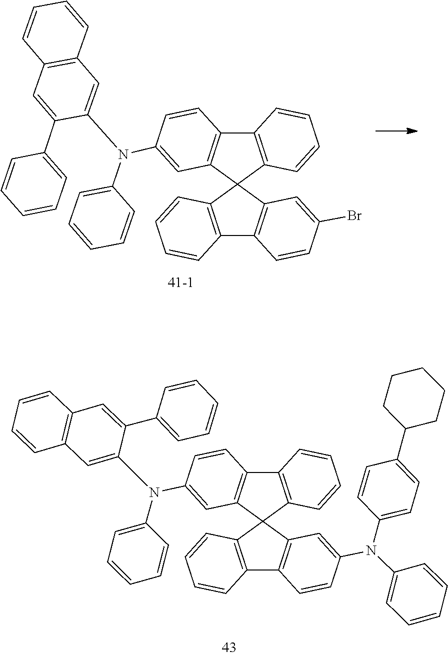 [SMILES: Brc1ccc2c(c1)C1(c3ccccc3-2)c2ccccc2-c2ccc(N(c3ccccc3)c3cc4ccccc4cc3-c3ccccc3)cc21 ; c1ccc(-c2cc3ccccc3cc2N(c2ccccc2)c2ccc3c(c2)C2(c4ccccc4-c4ccc(N(c5ccccc5)c5ccc(C6CCCCC6)cc5)cc42)c2ccccc2-3)cc1]

(Synthesis of Compound 43) Compound 41-1 (12 mmol, 1.2 equiv.), 4-cyclohexyl-N-phenylaniline (10 mmol, 1 equiv.), Pd₂(dba)₃ (0.5 mol, 0.05 equiv.), t-BuONa (20 mmol, 2 equiv.), t-Bu₃P (0.1 mmol, 0.1 equiv.), and 150 ml of Tol. were added to a one-neck round flask and stirred at 110° C. for an hour.

After the reaction was completed, the reaction product was worked up with ether/H₂O and purified by column chromatography using dichloromethane/hexane (1/8) to obtain 7.6 mmol of Compound 43 (yield=76%).

### Synthesis Example 14: Synthesis of Compound 45

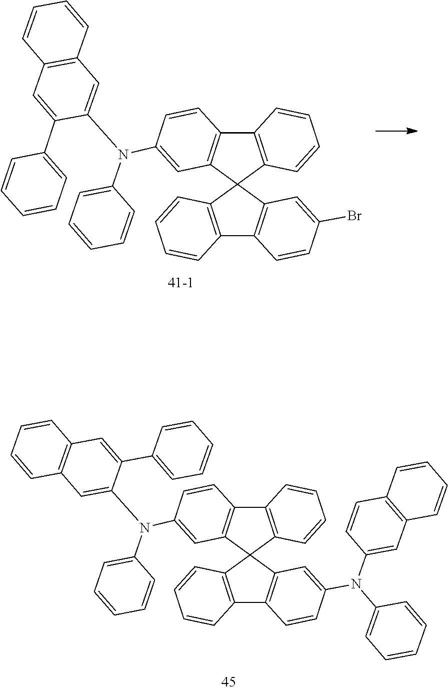 [SMILES: Brc1ccc2c(c1)C1(c3ccccc3-2)c2ccccc2-c2ccc(N(c3ccccc3)c3cc4ccccc4cc3-c3ccccc3)cc21 ; c1ccc(-c2cc3ccccc3cc2N(c2ccccc2)c2ccc3c(c2)C2(c4ccccc4-c4ccc(N(c5ccccc5)c5ccc6ccccc6c5)cc42)c2ccccc2-3)cc1]

(Synthesis of Compound 45) Compound 41-1 (12 mmol, 1.2 equiv.), N-phenylnaphthalen-2-amine (10 mmol, 1 equiv.), Pd₂(dba)₃ (0.5 mol, 0.05 equiv.), t-BuONa (20 mmol, 2 equiv.), t-Bu₃P (0.1 mmol, 0.1 equiv.), and 150 ml of Tol. were added to a one-neck round flask and stirred at 110° C. for an hour.

After the reaction was completed, the reaction product was worked up with ether/H₂O and purified by column chromatography using dichloromethane/hexane (1/8) to obtain 7.0 mmol of Compound 45 (yield=70%).

### Synthesis Example 15: Synthesis of Compound 47

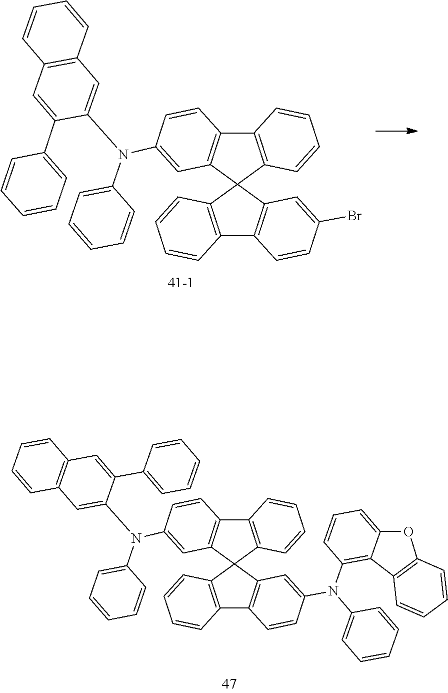 [SMILES: Brc1ccc2c(c1)C1(c3ccccc3-2)c2ccccc2-c2ccc(N(c3ccccc3)c3cc4ccccc4cc3-c3ccccc3)cc21 ; c1ccc(-c2cc3ccccc3cc2N(c2ccccc2)c2ccc3c(c2)C2(c4ccccc4-3)c3ccccc3-c3ccc(N(c4ccccc4)c4cccc5oc6ccccc6c45)cc32)cc1]

(Synthesis of Compound 47) Compound 41-1 (12 mmol, 1.2 equiv.), N-phenyldibenzo[b,d]furan-1-amine (10 mmol, 1 equiv.), Pd₂(dba)₃ (0.5 mol, 0.05 equiv.), t-BuONa (20 mmol, 2 equiv.), t-Bu₃P (0.1 mmol, 0.1 equiv.), and 150 ml of Tol. were added to a one-neck round flask and stirred at 110° C. for an hour.

After the reaction was completed, the reaction product was worked up with ether/H₂O and purified by column chromatography using dichloromethane/hexane (1/8) to obtain 7.0 mmol of Compound 47 (yield=70%).

### Synthesis Example 16: Synthesis of Compound 49

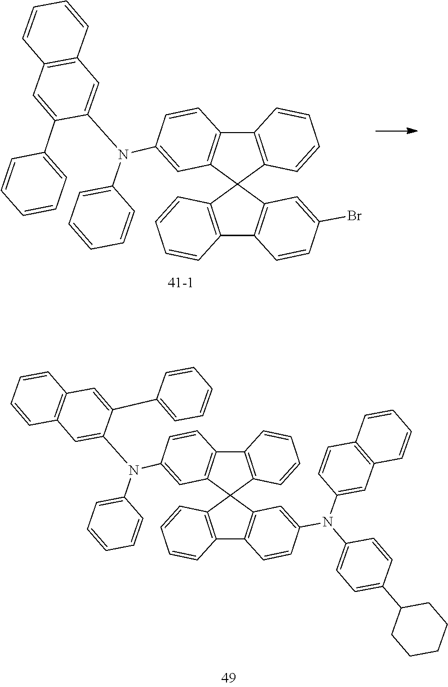 [SMILES: Brc1ccc2c(c1)C1(c3ccccc3-2)c2ccccc2-c2ccc(N(c3ccccc3)c3cc4ccccc4cc3-c3ccccc3)cc21 ; c1ccc(-c2cc3ccccc3cc2N(c2ccccc2)c2ccc3c(c2)C2(c4ccccc4-c4ccc(N(c5ccc(C6CCCCC6)cc5)c5ccc6ccccc6c5)cc42)c2ccccc2-3)cc1]

(Synthesis of Compound 49) Compound 41-1 (12 mmol, 1.2 equiv.), N-(4-cyclohexylphenyl) naphthalen-2-amine (10 mmol, 1 equiv.), Pd₂(dba)₃ (0.5 mol, 0.05 equiv.), t-BuONa (20 mmol, 2 equiv.), t-Bu₃P (0.1 mmol, 0.1 equiv.), and 150 ml of Tol. were added to a one-neck round flask and stirred at 110° C. for an hour.

After the reaction was completed, the reaction product was worked up with ether/H₂O and purified by column chromatography using dichloromethane/hexane (1/8) to obtain 6.9 mmol of Compound 49 (yield=69%).

### Synthesis Example 17: Synthesis of Compound 52

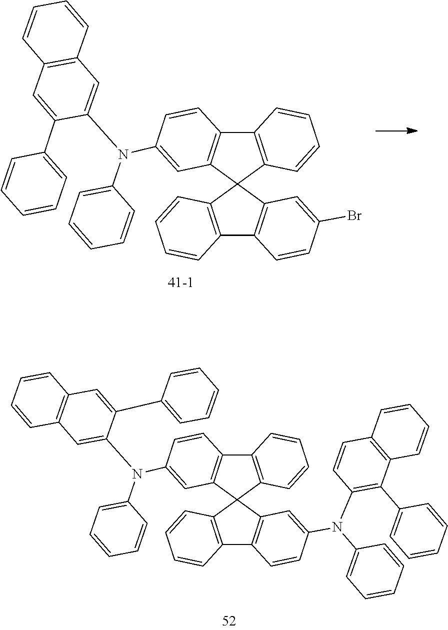 [SMILES: Brc1ccc2c(c1)C1(c3ccccc3-2)c2ccccc2-c2ccc(N(c3ccccc3)c3cc4ccccc4cc3-c3ccccc3)cc21 ; c1ccc(-c2cc3ccccc3cc2N(c2ccccc2)c2ccc3c(c2)C2(c4ccccc4-3)c3ccccc3-c3ccc(N(c4ccccc4)c4ccc5ccccc5c4-c4ccccc4)cc32)cc1]

(Synthesis of Compound 52) Compound 41-1 (12 mmol, 1.2 equiv.), N,1-diphenylnaphthalen-2-amine (10 mmol, 1 equiv.), Pd₂(dba)₃ (0.5 mol, 0.05 equiv.), t-BuONa (20 mmol, 2 equiv.), t-Bu₃P (0.1 mmol, 0.1 equiv.), and 150 ml of Tol. were added to a one-neck round flask and stirred at 110° C. for an hour.

After the reaction was completed, the reaction product was worked up with ether/H₂O and purified by column chromatography using dichloromethane/hexane (1/8) to obtain 6.8 mmol of Compound 52 (yield=68%).

### Synthesis Example 18: Synthesis of Compound 56

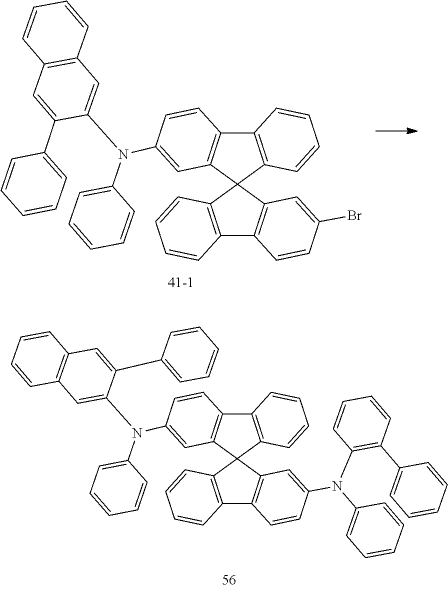 [SMILES: Brc1ccc2c(c1)C1(c3ccccc3-2)c2ccccc2-c2ccc(N(c3ccccc3)c3cc4ccccc4cc3-c3ccccc3)cc21 ; c1ccc(-c2ccccc2N(c2ccccc2)c2ccc3c(c2)C2(c4ccccc4-3)c3ccccc3-c3ccc(N(c4ccccc4)c4cc5ccccc5cc4-c4ccccc4)cc32)cc1]

(Synthesis of Compound 56) Compound 41-1 (12 mmol, 1.2 equiv.), N-phenyl-[1,1′-biphenyl]-2-amine (10 mmol, 1 equiv.), Pd₂(dba)₃ (0.5 mol, 0.05 equiv.), t-BuONa (20 mmol, 2 equiv.), t-Bu₃P (0.1 mmol, 0.1 equiv.), and 150 ml of Tol. were added to a one-neck round flask and stirred at 110° C. for an hour.

After the reaction was completed, the reaction product was worked up with ether/H₂O and purified by column chromatography using dichloromethane/hexane (1/8) to obtain 6.9 mmol of Compound 56 (yield=69%).

### Synthesis Example 19: Synthesis of Compound 73

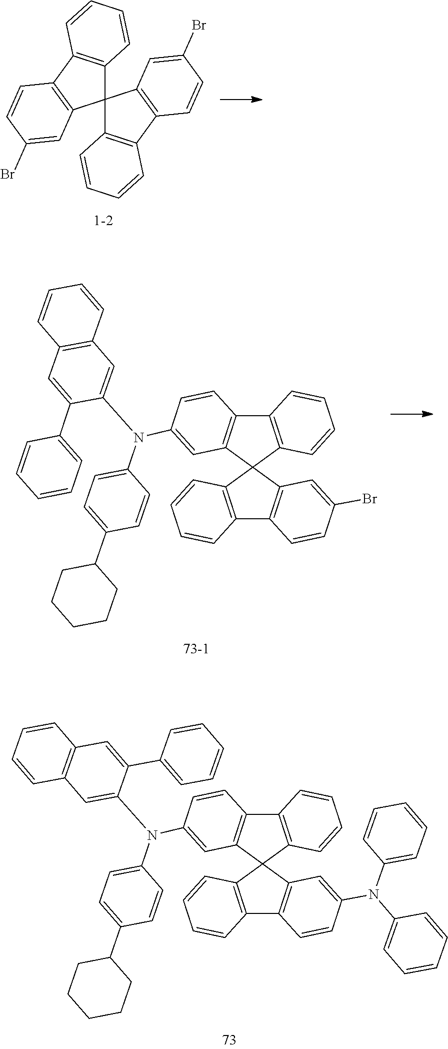 [SMILES: Brc1ccc2c(c1)C1(c3ccccc3-2)c2ccccc2-c2ccc(Br)cc21 ; Brc1ccc2c(c1)C1(c3ccccc3-2)c2ccccc2-c2ccc(N(c3ccc(C4CCCCC4)cc3)c3cc4ccccc4cc3-c3ccccc3)cc21 ; c1ccc(-c2cc3ccccc3cc2N(c2ccc(C3CCCCC3)cc2)c2ccc3c(c2)C2(c4ccccc4-c4ccc(N(c5ccccc5)c5ccccc5)cc42)c2ccccc2-3)cc1]

(Synthesis of Compound 73-1) 2,2′-dibromo-9,9′-spirobi [fluorene] (26 mmol, 1.3 equiv.), N-(4-cyclohexylphenyl)-3-phenylnaphthalen-2-amine (20 mmol, 1 equiv.), Pd₂(dba)₃ (1 mmol, 0.05 equiv.), t-BuONa (40 mmol, 2 equiv.), BINAP(2 mmol, 0.1 equiv.), and 300 ml of Tol. were added to a one-neck round flask and stirred at 110° C. for an hour.

After the reaction was completed, the reaction product was worked up with ether/H₂O and purified by column chromatography using dichloromethane/hexane (1/5) to obtain 15 mmol of Compound 73-1 (yield=75%).

(Synthesis of Compound 73) Compound 73-1 (12 mmol, 1.2 equiv.), diphenylamine (10 mmol, 1 equiv.), Pd₂(dba)₃ (0.5 mol, 0.05 equiv.), t-BuONa (20 mmol, 2 equiv.), t-Bu₃P (0.1 mmol, 0.1 equiv.), and 150 ml of Tol. were added to a one-neck round flask and stirred at 110° C. for an hour.

After the reaction was completed, the reaction product was worked up with ether/H₂O and purified by column chromatography using dichloromethane/hexane (1/8) to obtain 8.2 mmol of Compound 73 (yield=82%).

### Synthesis Example 20: Synthesis of Compound 77

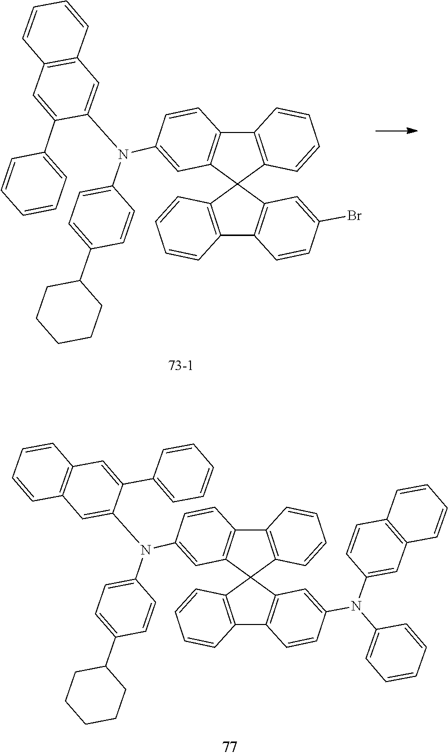 [SMILES: Brc1ccc2c(c1)C1(c3ccccc3-2)c2ccccc2-c2ccc(N(c3ccc(C4CCCCC4)cc3)c3cc4ccccc4cc3-c3ccccc3)cc21 ; c1ccc(-c2cc3ccccc3cc2N(c2ccc(C3CCCCC3)cc2)c2ccc3c(c2)C2(c4ccccc4-c4ccc(N(c5ccccc5)c5ccc6ccccc6c5)cc42)c2ccccc2-3)cc1]

(Synthesis of Compound 77) Compound 73-1 (12 mmol, 1.2 equiv.), N-phenylnaphthalen-2-amine (10 mmol, 1 equiv.), Pd₂(dba)₃ (0.5 mol, 0.05 equiv.), t-BuONa (20 mmol, 2 equiv.), t-Bu₃P (0.1 mmol, 0.1 equiv.), and 150 ml of Tol. were added to a one-neck round flask and stirred at 110° C. for an hour.

After the reaction was completed, the reaction product was worked up with ether/H₂O and purified by column chromatography using dichloromethane/hexane (1/8) to obtain 7.2 mmol of Compound 77 (yield=72%).

### Synthesis Example 21: Synthesis of Compound 101

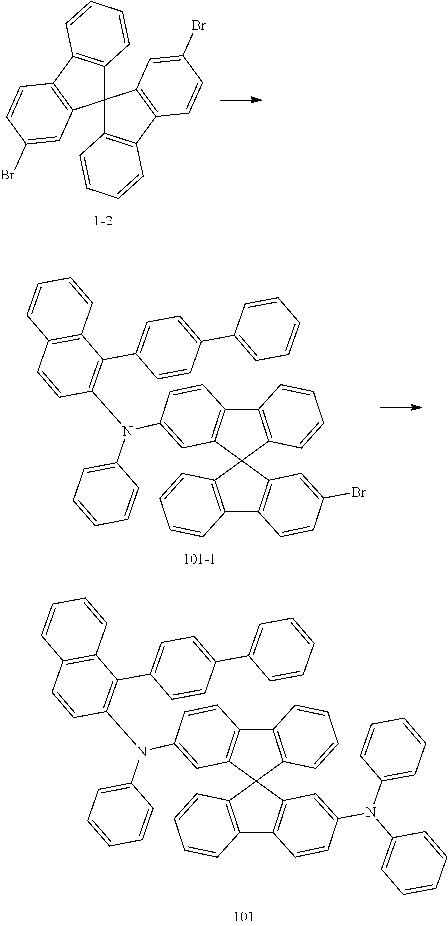 [SMILES: Brc1ccc2c(c1)C1(c3ccccc3-2)c2ccccc2-c2ccc(Br)cc21 ; Brc1ccc2c(c1)C1(c3ccccc3-2)c2ccccc2-c2ccc(N(c3ccccc3)c3ccc4ccccc4c3-c3ccc(-c4ccccc4)cc3)cc21 ; c1ccc(-c2ccc(-c3c(N(c4ccccc4)c4ccc5c(c4)C4(c6ccccc6-c6ccc(N(c7ccccc7)c7ccccc7)cc64)c4ccccc4-5)ccc4ccccc34)cc2)cc1]

(Synthesis of Compound 101-1) 2,2′-dibromo-9,9′-spirobi [fluorene] (26 mmol, 1.3 equiv.) 1-([1,1′-biphenyl]-4-yl)—N-phenylnaphthalen-2-amine (20 mmol, 1 equiv.), Pd₂(dba)₃ (1 mmol, 0.05 equiv.), t-BuONa (40 mmol, 2 equiv.), BINAP(2 mmol, 0.1 equiv.), and 300 ml of Tol. were added to a one-neck round flask and stirred at 110° C. for an hour.

After the reaction was completed, the reaction product was worked up with ether/H₂O and purified by column chromatography using dichloromethane/hexane (1/5) to obtain 14 mmol of Compound 101-1 (yield=70%).

(Synthesis of Compound 101) Compound 101-1 (12 mmol, 1.2 equiv.), diphenylamine (10 mmol, 1 equiv.), Pd₂(dba)₃ (0.5 mol, 0.05 equiv.), t-BuONa (20 mmol, 2 equiv.), t-Bu₃P (0.1 mmol, 0.1 equiv.), and 150 ml of Tol. were added to a one-neck round flask and stirred at 110° C. for an hour.

After the reaction was completed, the reaction product was worked up with ether/H₂O and purified by column chromatography using dichloromethane/hexane (1/8) to obtain 8.0 mmol of Compound 101 (yield=80%).

### Synthesis Example 22: Synthesis of Compound 102

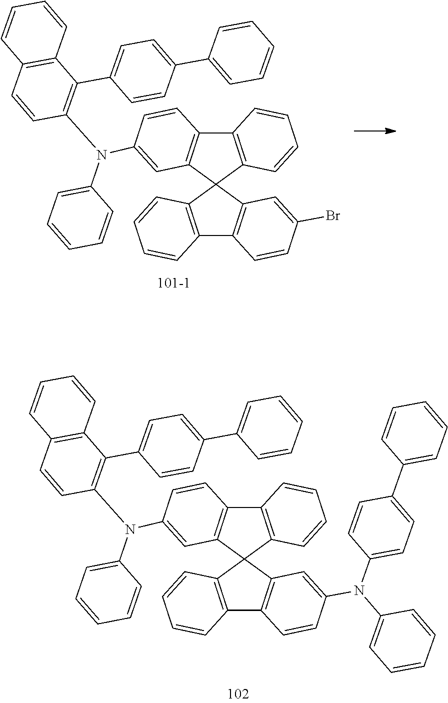 [SMILES: Brc1ccc2c(c1)C1(c3ccccc3-2)c2ccccc2-c2ccc(N(c3ccccc3)c3ccc4ccccc4c3-c3ccc(-c4ccccc4)cc3)cc21 ; c1ccc(-c2ccc(-c3c(N(c4ccccc4)c4ccc5c(c4)C4(c6ccccc6-c6ccc(N(c7ccccc7)c7ccc(-c8ccccc8)cc7)cc64)c4ccccc4-5)ccc4ccccc34)cc2)cc1]

(Synthesis of Compound 102) Compound 101-1 (12 mmol, 1.2 equiv.), N-phenyl-[1,1′-biphenyl]-4-amine (10 mmol, 1 equiv.), Pd₂(dba)₃ (0.5 mol, 0.05 equiv.), t-BuONa (20 mmol, 2 equiv.), t-Bu₃P (0.1 mmol, 0.1 equiv.), and 150 ml of Tol. were added to a one-neck round flask and stirred at 110° C. for an hour.

After the reaction was completed, the reaction product was worked up with ether/H₂O and purified by column chromatography using dichloromethane/hexane (1/8) to obtain 7.9 mmol of Compound 102 (yield=79%).

### Synthesis Example 23: Synthesis of Compound 103

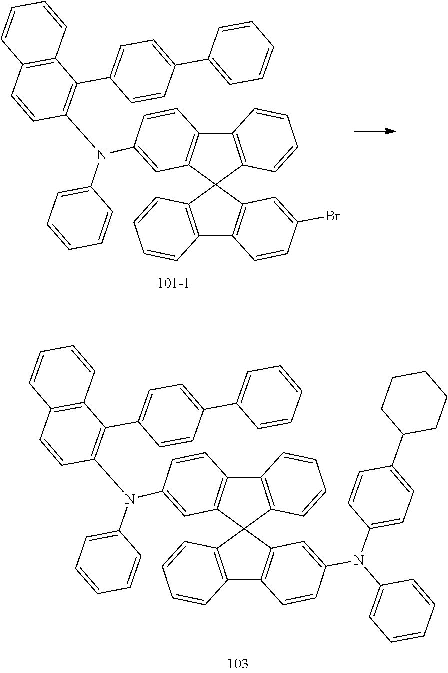 [SMILES: Brc1ccc2c(c1)C1(c3ccccc3-2)c2ccccc2-c2ccc(N(c3ccccc3)c3ccc4ccccc4c3-c3ccc(-c4ccccc4)cc3)cc21 ; c1ccc(-c2ccc(-c3c(N(c4ccccc4)c4ccc5c(c4)C4(c6ccccc6-c6ccc(N(c7ccccc7)c7ccc(C8CCCCC8)cc7)cc64)c4ccccc4-5)ccc4ccccc34)cc2)cc1]

(Synthesis of Compound 103) Compound 101-1 (12 mmol, 1.2 equiv.), 4-cyclohexyl-N-phenylaniline (10 mmol, 1 equiv.), Pd₂(dba)₃ (0.5 mol, 0.05 equiv.), t-BuONa (20 mmol, 2 equiv.), t-Bu₃P (0.1 mmol, 0.1 equiv.), and 150 ml of Tol. were added to a one-neck round flask and stirred at 110° C. for an hour.

After the reaction was completed, the reaction product was worked up with ether/H₂O and purified by column chromatography using dichloromethane/hexane (1/8) to obtain 7.5 mmol of Compound 103 (yield=75%).

### Synthesis Example 24: Synthesis of Compound 141

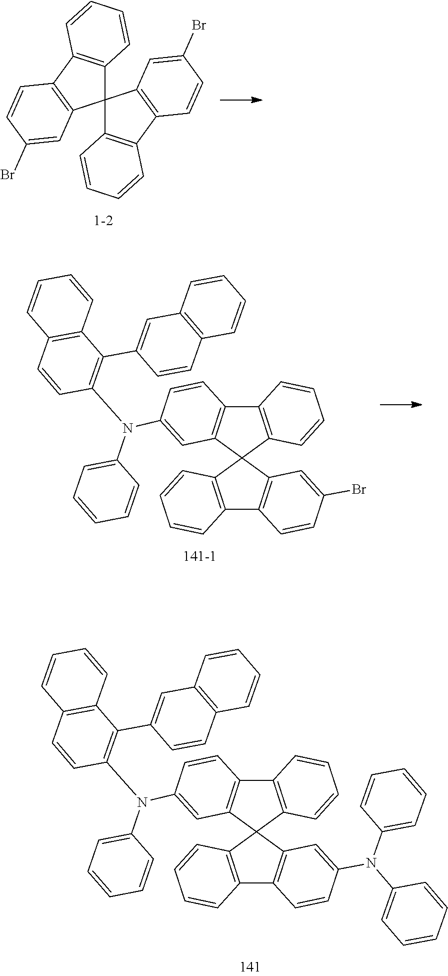 [SMILES: Brc1ccc2c(c1)C1(c3ccccc3-2)c2ccccc2-c2ccc(Br)cc21 ; Brc1ccc2c(c1)C1(c3ccccc3-2)c2ccccc2-c2ccc(N(c3ccccc3)c3ccc4ccccc4c3-c3ccc4ccccc4c3)cc21 ; c1ccc(N(c2ccccc2)c2ccc3c(c2)C2(c4ccccc4-3)c3ccccc3-c3ccc(N(c4ccccc4)c4ccc5ccccc5c4-c4ccc5ccccc5c4)cc32)cc1]

(Synthesis of Compound 141-1) 2,2′-dibromo-9,9′-spirobi [fluorene] (26 mmol, 1.3 equiv.)N-phenyl-[1,2′-binaphthalen]-2-amine (20 mmol, 1 equiv.), Pd₂(dba)₃ (1 mmol, 0.05 equiv.), t-BuONa (40 mmol, 2 equiv.), BINAP(2 mmol, 0.1 equiv.), and 300 ml of Tol. were added to a one-neck round flask and stirred at 110° C. for an hour.

After the reaction was completed, the reaction product was worked up with ether/H₂O and purified by column chromatography using dichloromethane/hexane (1/5) to obtain 13 mmol of Compound 141-1 (yield=65%).

(Synthesis of Compound 141) Compound 141-1 (12 mmol, 1.2 equiv.), diphenylamine (10 mmol, 1 equiv.), Pd₂(dba)₃ (0.5 mol, 0.05 equiv.), t-BuONa (20 mmol, 2 equiv.), t-Bu₃P (0.1 mmol, 0.1 equiv.), and 150 ml of Tol. were added to a one-neck round flask and stirred at 110° C. for an hour.

After the reaction was completed, the reaction product was worked up with ether/H₂O and purified by column chromatography using dichloromethane/hexane (1/8) to obtain 8.0 mmol of Compound 141 (yield=80%).

### Synthesis Example 25: Synthesis of Compound 142

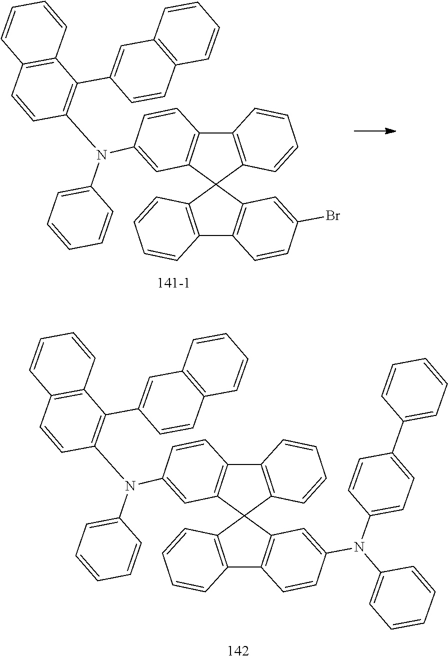 [SMILES: Brc1ccc2c(c1)C1(c3ccccc3-2)c2ccccc2-c2ccc(N(c3ccccc3)c3ccc4ccccc4c3-c3ccc4ccccc4c3)cc21 ; c1ccc(-c2ccc(N(c3ccccc3)c3ccc4c(c3)C3(c5ccccc5-4)c4ccccc4-c4ccc(N(c5ccccc5)c5ccc6ccccc6c5-c5ccc6ccccc6c5)cc43)cc2)cc1]

(Synthesis of Compound 142) Compound 141-1 (12 mmol, 1.2 equiv.), N-phenyl-[1,1′-biphenyl]-4-amine (10 mmol, 1 equiv.), Pd₂(dba)₃ (0.5 mol, 0.05 equiv.), t-BuONa (20 mmol, 2 equiv.), t-Bu₃P (0.1 mmol, 0.1 equiv.), and 150 ml of Tol. were added to a one-neck round flask and stirred at 110° C. for an hour.

After the reaction was completed, the reaction product was worked up with ether/H₂O and purified by column chromatography using dichloromethane/hexane (1/8) to obtain 7.4 mmol of Compound 142 (yield=74%).

### Synthesis Example 26: Synthesis of Compound 161

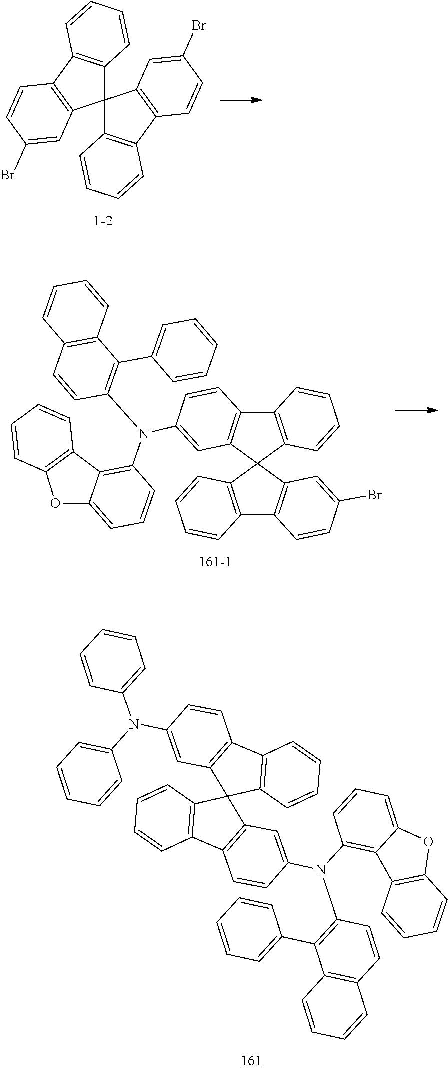 [SMILES: Brc1ccc2c(c1)C1(c3ccccc3-2)c2ccccc2-c2ccc(Br)cc21 ; Brc1ccc2c(c1)C1(c3ccccc3-2)c2ccccc2-c2ccc(N(c3ccc4ccccc4c3-c3ccccc3)c3cccc4oc5ccccc5c34)cc21 ; c1ccc(-c2c(N(c3ccc4c(c3)C3(c5ccccc5-c5ccc(N(c6ccccc6)c6ccccc6)cc53)c3ccccc3-4)c3cccc4oc5ccccc5c34)ccc3ccccc23)cc1]

(Synthesis of Compound 161-1) 2,2′-dibromo-9,9′-spirobi [fluorene] (26 mmol, 1.3 equiv.)N-(1-phenylnaphthalen-2-yl)dibenzo[b,d]furan-1-amine (20 mmol, 1 equiv.), Pd₂(dba)₃ (1 mmol, 0.05 equiv.), t-BuONa (40 mmol, 2 equiv.), BINAP(2 mmol, 0.1 equiv.), and 300 ml of Tol. were added to a one-neck round flask and stirred at 110° C. for an hour.

After the reaction was completed, the reaction product was worked up with ether/H₂O and purified by column chromatography using dichloromethane/hexane (1/5) to obtain 15 mmol of Compound 161-1 (yield=75%).

(Synthesis of Compound 161) Compound 161-1 (12 mmol, 1.2 equiv.), diphenylamine (10 mmol, 1 equiv.), Pd₂(dba)₃ (0.5 mol, 0.05 equiv.), t-BuONa (20 mmol, 2 equiv.), t-Bu₃P (0.1 mmol, 0.1 equiv.), and 150 ml of Tol. were added to a one-neck round flask and stirred at 110° C. for an hour.

After the reaction was completed, the reaction product was worked up with ether/H₂O and purified by column chromatography using dichloromethane/hexane (1/8) to obtain 8.0 mmol of Compound 161 (yield=80%).

### Synthesis Example 27: Synthesis of Compound 162

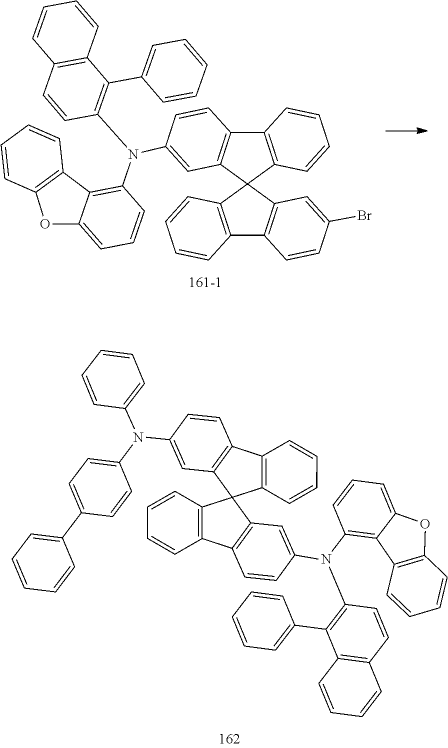 [SMILES: Brc1ccc2c(c1)C1(c3ccccc3-2)c2ccccc2-c2ccc(N(c3ccc4ccccc4c3-c3ccccc3)c3cccc4oc5ccccc5c34)cc21 ; c1ccc(-c2ccc(N(c3ccccc3)c3ccc4c(c3)C3(c5ccccc5-4)c4ccccc4-c4ccc(N(c5ccc6ccccc6c5-c5ccccc5)c5cccc6oc7ccccc7c56)cc43)cc2)cc1]

(Synthesis of Compound 162) Compound 161-1 (12 mmol, 1.2 equiv.), N-phenyl-[1,1′-biphenyl]-4-amine (10 mmol, 1 equiv.), Pd₂(dba)₃ (0.5 mol, 0.05 equiv.), t-BuONa (20 mmol, 2 equiv.), t-Bu₃P (0.1 mmol, 0.1 equiv.), and 150 ml of Tol. were added to a one-neck round flask and stirred at 110° C. for an hour.

After the reaction was completed, the reaction product was worked up with ether/H₂O and purified by column chromatography using dichloromethane/hexane (1/8) to obtain 7.6 mmol of Compound 162 (yield=76%).

### Example 1

As an anode, a glass substrate (product of Corning Inc.) with a 15 Ω/cm² (1,200 Å) ITO electrode formed thereon was cut to a size of 50 mm×50 mm×0.7 mm, sonicated with isopropyl alcohol and pure water each for 5 minutes, cleaned by irradiation of ultraviolet rays and ozone exposure for 30 minutes, and mounted on a vacuum deposition apparatus.

NPD was deposited on the anode to form a hole injection layer having a thickness of 300 Å, Compound 1 was deposited on the hole injection layer to form a hole transport layer having a thickness of 200 Å, and CzSi was deposited on the hole transport layer to form an emission auxiliary layer having a thickness of 100 Å.

HTH33+ETH2 (host), PD39 (phosphorescence sensitizer), and t-DABNA (boron dopant) were co-deposited on the emission auxiliary layer at a weight ratio of 42:42:15:1 to form an emission layer having a thickness of 200 Å, TSPO1 was deposited on the emission layer to form a hole blocking layer having a thickness of 200 Å, TPBI was deposited on the hole blocking to form an electron transport layer having a thickness of 300 Å, LiF was deposited on the electron transport layer to form an electron injection layer having a thickness of 10 Å, and Al was deposited on the electron injection layer to form a cathode having a thickness of 3,000 Å, thereby manufacturing an organic light-emitting device.

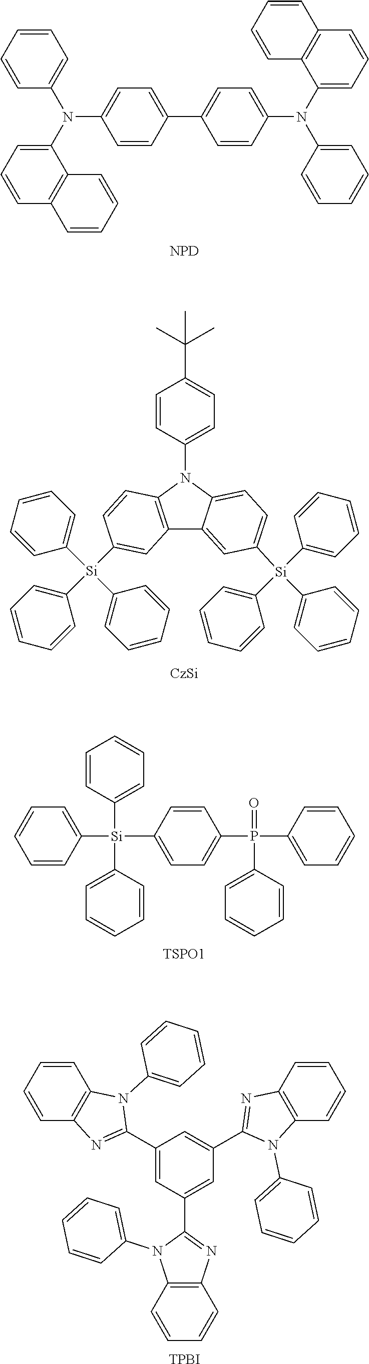

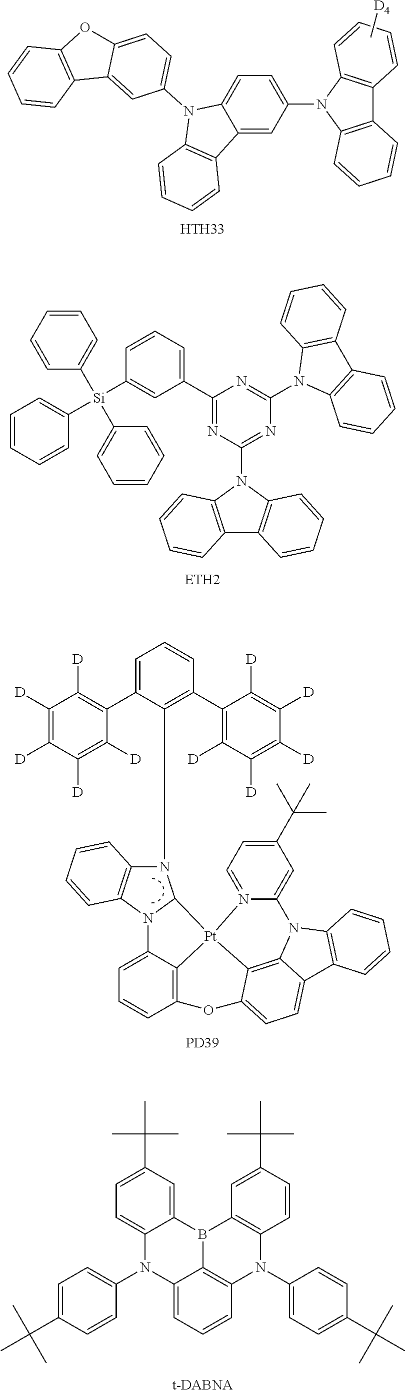

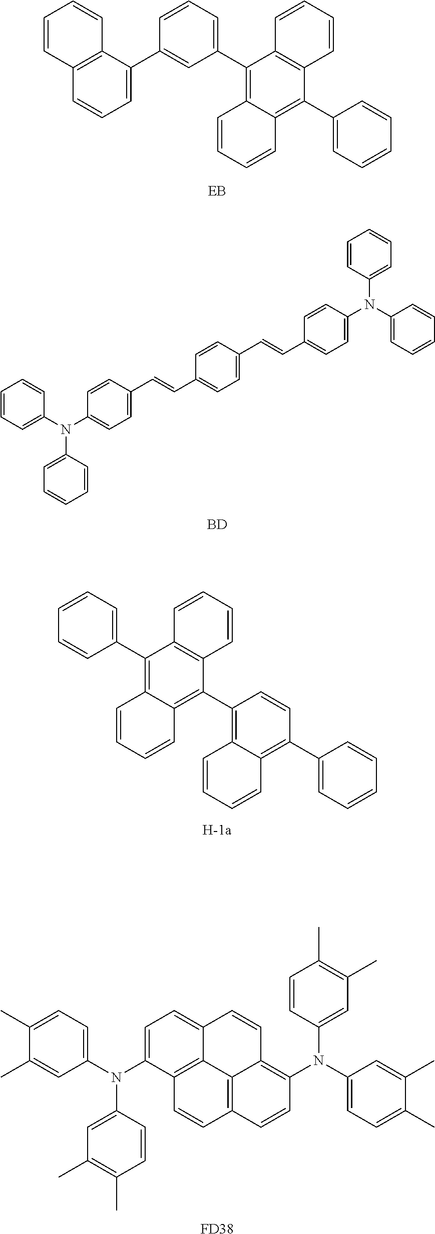

### Examples 2 to 27 and Comparative Examples 1 to 6

Organic light-emitting devices were manufactured in substantially the same manner as in Example 1, except that Compounds shown in Table 1 were each used instead of Compound 1 in forming a hole transport layer.

### Comparative Example 7

An organic light-emitting device was manufactured in substantially the same manner as in Example 1, except that C7 was used instead of Compound 1 in forming a hole transport layer and Host EB (4% Dopant BD) was used in an emission layer.

### Comparative Example 8

An organic light-emitting device was manufactured in substantially the same manner as in Example 1, except that C3 was used in forming a hole transport layer, C8 was deposited thereon to form an emission auxiliary layer having a thickness of 100 Å, and Compound H1-a (host) and Compound FD38 (fluorescent dopant) were co-deposited on the emission auxiliary layer at a weight ratio of 95:5 to form an emission layer having a thickness of 200 Å.

### Evaluation Example 1

The driving voltage, luminescence efficiency, emission wavelength, and lifespan of the light-emitting devices manufactured according to Examples 1 to 27 and Comparative Examples 1 to 8 were measured using a Keithley SMU 236 and a luminance meter PR650. Results thereof are shown in Table 1.

TABLE 1

| | Hole transport layer | Driving | | Emission | |
|---|---|---|---|---|---|
| | compound (Emission | voltage | Efficiency | wavelength | Lifespan |
| | auxiliary/host/dopant) | (V) | (cd/A) | (nm) | (T95) |
| Comparative | C3 | 4.4 | 26.3 | 462 | 220 |
| Example 1 | | | | | |
| Comparative | C1 | 4.18 | 27.5 | 461 | 240 |
| Example 2 | | | | | |
| Comparative | C2 | 4.20 | 25.0 | 460 | 200 |
| Example 3 | | | | | |
| Comparative | C4 | 4.05 | 28.5 | 460 | 270 |
| Example 4 | | | | | |
| Comparative | C5 | 4.62 | 23.0 | 460 | 200 |
| Example 5 | | | | | |
| Comparative | C6 | 4.75 | 23.5 | 460 | 175 |
| Example 6 | | | | | |
| Comparative | C7 (CzSi/EB/BD) | 4.50 | 21.0 | 460 | 185 |
| Example 7 | | | | | |
| Comparative | C3 (C8/H-1a/FD38) | 4.57 | 22.5 | 461 | 160 |
| Example 8 | | | | | |
| Example 1 | Compound 1 | 3.55 | 30.5 | 460 | 360 |
| Example 2 | Compound 2 | 3.61 | 30.2 | 460 | 350 |
| Example 3 | Compound 3 | 3.65 | 32.2 | 460 | 365 |
| Example 4 | Compound 5 | 3.64 | 31.4 | 462 | 345 |
| Example 5 | Compound 7 | 3.57 | 30.6 | 459 | 340 |
| Example 6 | Compound 9 | 3.54 | 30.4 | 459 | 380 |
| Example 7 | Compound 12 | 3.54 | 30.5 | 459 | 360 |
| Example 8 | Compound 16 | 3.61 | 31.5 | 460 | 365 |
| Example 9 | Compound 33 | 3.64 | 31.6 | 460 | 370 |
| Example 10 | Compound 37 | 3.59 | 33.0 | 461 | 350 |
| Example 11 | Compound 41 | 3.48 | 32.8 | 460 | 350 |
| Example 12 | Compound 42 | 3.52 | 32.7 | 460 | 345 |
| Example 13 | Compound 43 | 3.52 | 30.6 | 461 | 330 |
| Example 14 | Compound 45 | 3.55 | 31.7 | 462 | 360 |
| Example 15 | Compound 47 | 3.65 | 30.6 | 462 | 360 |
| Example 16 | Compound 49 | 3.57 | 31.3 | 460 | 360 |
| Example 17 | Compound 52 | 3.53 | 31.4 | 460 | 370 |
| Example 18 | Compound 56 | 3.54 | 31.5 | 460 | 360 |
| Example 19 | Compound 73 | 3.61 | 32.0 | 459 | 360 |
| Example 20 | Compound 77 | 3.60 | 30.9 | 460 | 355 |
| Example 21 | Compound 101 | 3.59 | 31.2 | 460 | 350 |
| Example 22 | Compound 102 | 3.48 | 30.8 | 462 | 380 |
| Example 23 | Compound 103 | 3.52 | 30.9 | 462 | 355 |
| Example 24 | Compound 141 | 3.52 | 33.0 | 461 | 360 |
| Example 25 | Compound 142 | 3.55 | 33.0 | 461 | 370 |
| Example 26 | Compound 161 | 3.57 | 31.3 | 460 | 360 |
| Example 27 | Compound 162 | 3.53 | 31.4 | 460 | 370 |

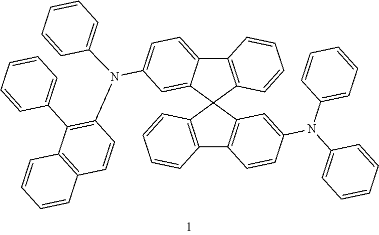 [SMILES: c1ccc(-c2c(N(c3ccccc3)c3ccc4c(c3)C3(c5ccccc5-c5ccc(N(c6ccccc6)c6ccccc6)cc53)c3ccccc3-4)ccc3ccccc23)cc1]

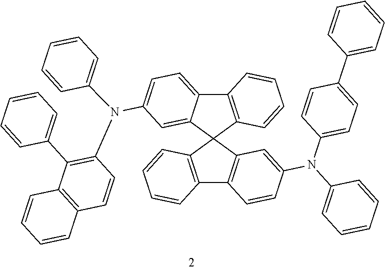 [SMILES: c1ccc(-c2ccc(N(c3ccccc3)c3ccc4c(c3)C3(c5ccccc5-4)c4ccccc4-c4ccc(N(c5ccccc5)c5ccc6ccccc6c5-c5ccccc5)cc43)cc2)cc1]

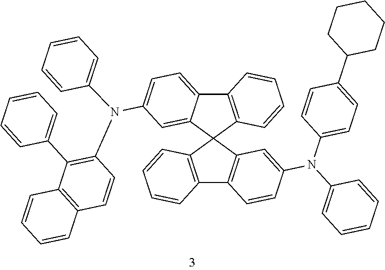 [SMILES: c1ccc(-c2c(N(c3ccccc3)c3ccc4c(c3)C3(c5ccccc5-c5ccc(N(c6ccccc6)c6ccc(C7CCCCC7)cc6)cc53)c3ccccc3-4)ccc3ccccc23)cc1]

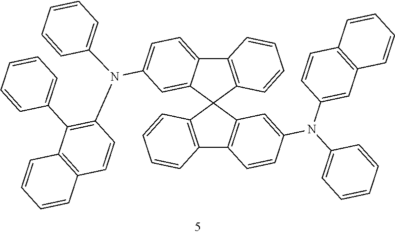 [SMILES: c1ccc(-c2c(N(c3ccccc3)c3ccc4c(c3)C3(c5ccccc5-c5ccc(N(c6ccccc6)c6ccc7ccccc7c6)cc53)c3ccccc3-4)ccc3ccccc23)cc1]

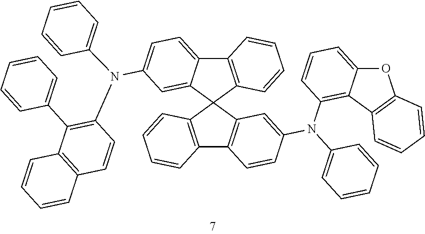 [SMILES: c1ccc(-c2c(N(c3ccccc3)c3ccc4c(c3)C3(c5ccccc5-4)c4ccccc4-c4ccc(N(c5ccccc5)c5cccc6oc7ccccc7c56)cc43)ccc3ccccc23)cc1]

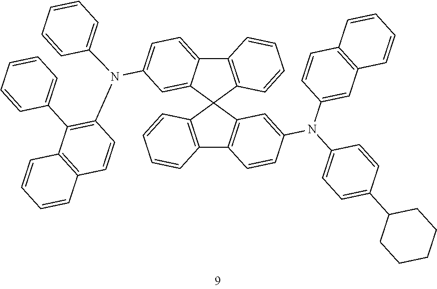 [SMILES: c1ccc(-c2c(N(c3ccccc3)c3ccc4c(c3)C3(c5ccccc5-c5ccc(N(c6ccc(C7CCCCC7)cc6)c6ccc7ccccc7c6)cc53)c3ccccc3-4)ccc3ccccc23)cc1]

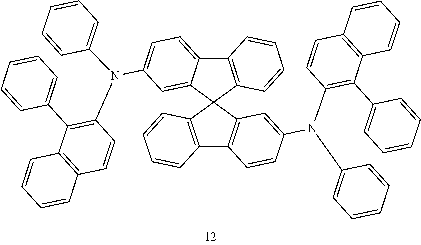 [SMILES: c1ccc(-c2c(N(c3ccccc3)c3ccc4c(c3)C3(c5ccccc5-4)c4ccccc4-c4ccc(N(c5ccccc5)c5ccc6ccccc6c5-c5ccccc5)cc43)ccc3ccccc23)cc1]

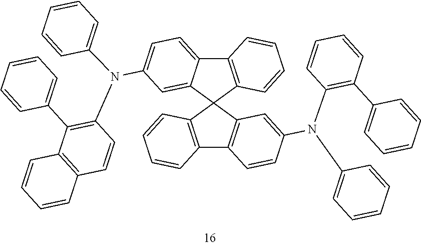 [SMILES: c1ccc(-c2ccccc2N(c2ccccc2)c2ccc3c(c2)C2(c4ccccc4-3)c3ccccc3-c3ccc(N(c4ccccc4)c4ccc5ccccc5c4-c4ccccc4)cc32)cc1]

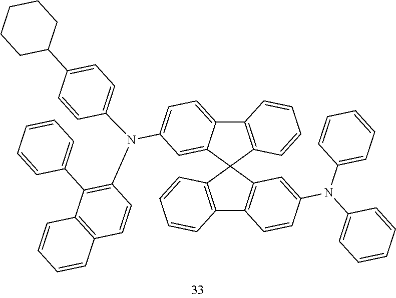 [SMILES: c1ccc(-c2c(N(c3ccc(C4CCCCC4)cc3)c3ccc4c(c3)C3(c5ccccc5-c5ccc(N(c6ccccc6)c6ccccc6)cc53)c3ccccc3-4)ccc3ccccc23)cc1]

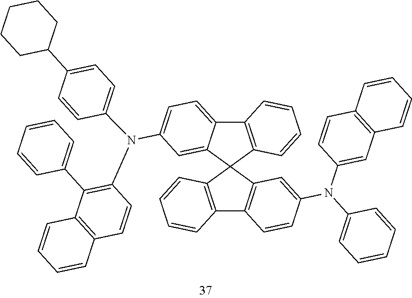 [SMILES: c1ccc(-c2c(N(c3ccc(C4CCCCC4)cc3)c3ccc4c(c3)C3(c5ccccc5-c5ccc(N(c6ccccc6)c6ccc7ccccc7c6)cc53)c3ccccc3-4)ccc3ccccc23)cc1]

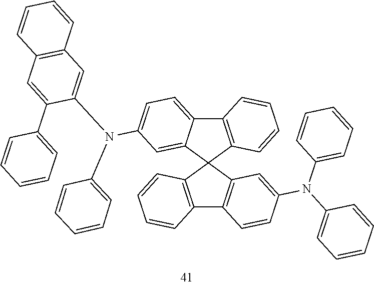 [SMILES: c1ccc(-c2cc3ccccc3cc2N(c2ccccc2)c2ccc3c(c2)C2(c4ccccc4-c4ccc(N(c5ccccc5)c5ccccc5)cc42)c2ccccc2-3)cc1]

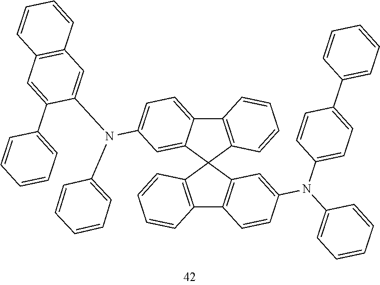 [SMILES: c1ccc(-c2ccc(N(c3ccccc3)c3ccc4c(c3)C3(c5ccccc5-4)c4ccccc4-c4ccc(N(c5ccccc5)c5cc6ccccc6cc5-c5ccccc5)cc43)cc2)cc1]

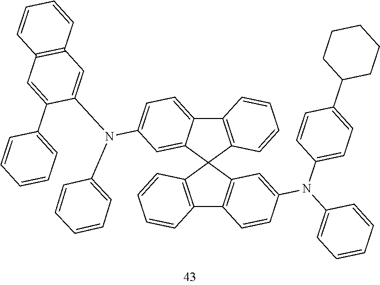 [SMILES: c1ccc(-c2cc3ccccc3cc2N(c2ccccc2)c2ccc3c(c2)C2(c4ccccc4-c4ccc(N(c5ccccc5)c5ccc(C6CCCCC6)cc5)cc42)c2ccccc2-3)cc1]

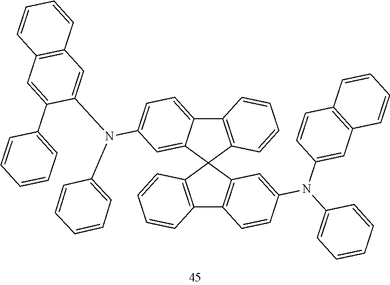 [SMILES: c1ccc(-c2cc3ccccc3cc2N(c2ccccc2)c2ccc3c(c2)C2(c4ccccc4-c4ccc(N(c5ccccc5)c5ccc6ccccc6c5)cc42)c2ccccc2-3)cc1]

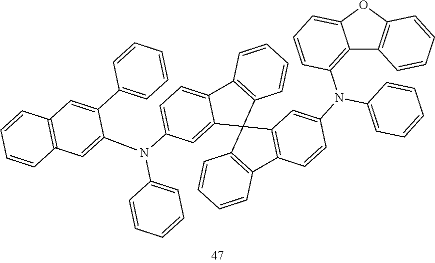 [SMILES: c1ccc(-c2cc3ccccc3cc2N(c2ccccc2)c2ccc3c(c2)C2(c4ccccc4-3)c3ccccc3-c3ccc(N(c4ccccc4)c4cccc5oc6ccccc6c45)cc32)cc1]

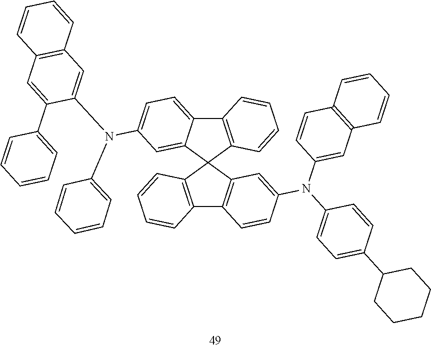 [SMILES: c1ccc(-c2cc3ccccc3cc2N(c2ccccc2)c2ccc3c(c2)C2(c4ccccc4-c4ccc(N(c5ccc(C6CCCCC6)cc5)c5ccc6ccccc6c5)cc42)c2ccccc2-3)cc1]

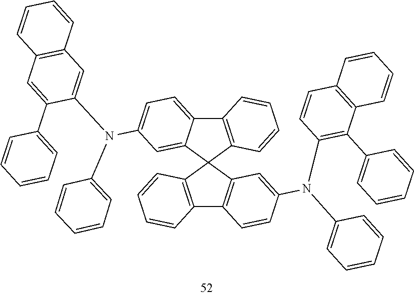 [SMILES: c1ccc(-c2cc3ccccc3cc2N(c2ccccc2)c2ccc3c(c2)C2(c4ccccc4-3)c3ccccc3-c3ccc(N(c4ccccc4)c4ccc5ccccc5c4-c4ccccc4)cc32)cc1]

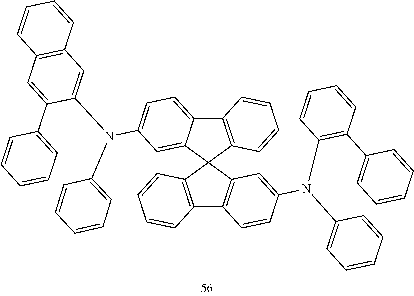 [SMILES: c1ccc(-c2ccccc2N(c2ccccc2)c2ccc3c(c2)C2(c4ccccc4-3)c3ccccc3-c3ccc(N(c4ccccc4)c4cc5ccccc5cc4-c4ccccc4)cc32)cc1]

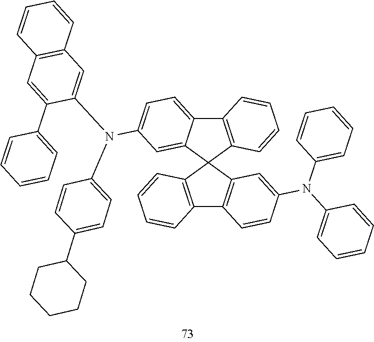 [SMILES: c1ccc(-c2cc3ccccc3cc2N(c2ccc(C3CCCCC3)cc2)c2ccc3c(c2)C2(c4ccccc4-c4ccc(N(c5ccccc5)c5ccccc5)cc42)c2ccccc2-3)cc1]

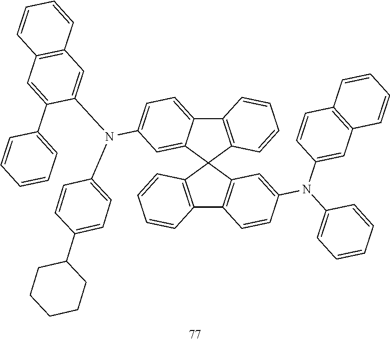 [SMILES: c1ccc(-c2cc3ccccc3cc2N(c2ccc(C3CCCCC3)cc2)c2ccc3c(c2)C2(c4ccccc4-c4ccc(N(c5ccccc5)c5ccc6ccccc6c5)cc42)c2ccccc2-3)cc1]

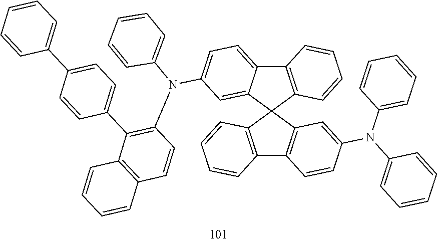 [SMILES: c1ccc(-c2ccc(-c3c(N(c4ccccc4)c4ccc5c(c4)C4(c6ccccc6-c6ccc(N(c7ccccc7)c7ccccc7)cc64)c4ccccc4-5)ccc4ccccc34)cc2)cc1]

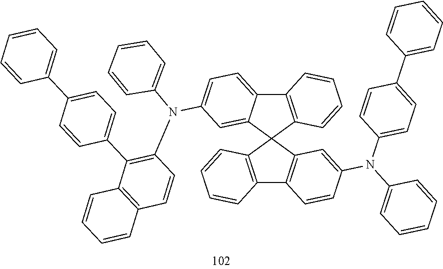 [SMILES: c1ccc(-c2ccc(-c3c(N(c4ccccc4)c4ccc5c(c4)C4(c6ccccc6-c6ccc(N(c7ccccc7)c7ccc(-c8ccccc8)cc7)cc64)c4ccccc4-5)ccc4ccccc34)cc2)cc1]

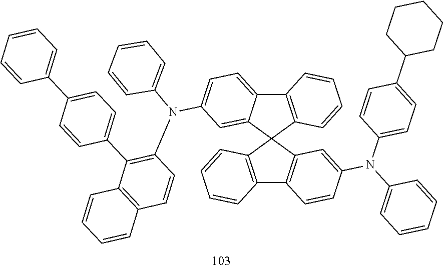 [SMILES: c1ccc(-c2ccc(-c3c(N(c4ccccc4)c4ccc5c(c4)C4(c6ccccc6-c6ccc(N(c7ccccc7)c7ccc(C8CCCCC8)cc7)cc64)c4ccccc4-5)ccc4ccccc34)cc2)cc1]

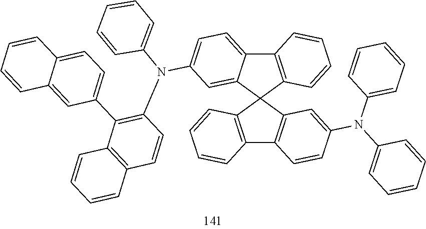 [SMILES: c1ccc(N(c2ccccc2)c2ccc3c(c2)C2(c4ccccc4-3)c3ccccc3-c3ccc(N(c4ccccc4)c4ccc5ccccc5c4-c4ccc5ccccc5c4)cc32)cc1]

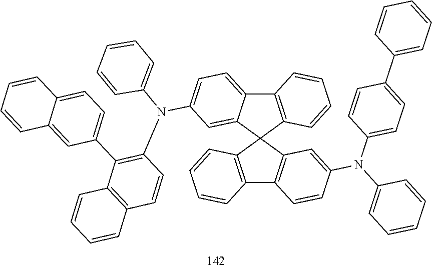 [SMILES: c1ccc(-c2ccc(N(c3ccccc3)c3ccc4c(c3)C3(c5ccccc5-4)c4ccccc4-c4ccc(N(c5ccccc5)c5ccc6ccccc6c5-c5ccc6ccccc6c5)cc43)cc2)cc1]

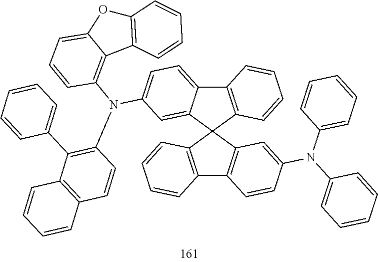 [SMILES: c1ccc(-c2c(N(c3ccc4c(c3)C3(c5ccccc5-c5ccc(N(c6ccccc6)c6ccccc6)cc53)c3ccccc3-4)c3cccc4oc5ccccc5c34)ccc3ccccc23)cc1]

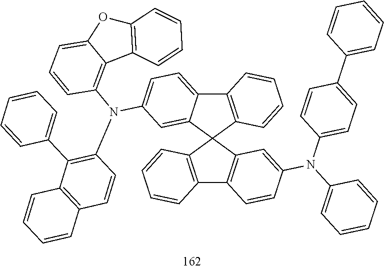 [SMILES: c1ccc(-c2ccc(N(c3ccccc3)c3ccc4c(c3)C3(c5ccccc5-4)c4ccccc4-c4ccc(N(c5ccc6ccccc6c5-c5ccccc5)c5cccc6oc7ccccc7c56)cc43)cc2)cc1]

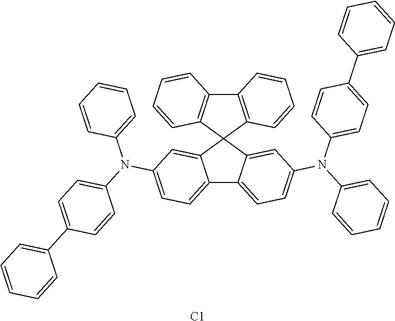 [SMILES: c1ccc(-c2ccc(N(c3ccccc3)c3ccc4c(c3)C3(c5ccccc5-c5ccccc53)c3cc(N(c5ccccc5)c5ccc(-c6ccccc6)cc5)ccc3-4)cc2)cc1 ; "C1"]

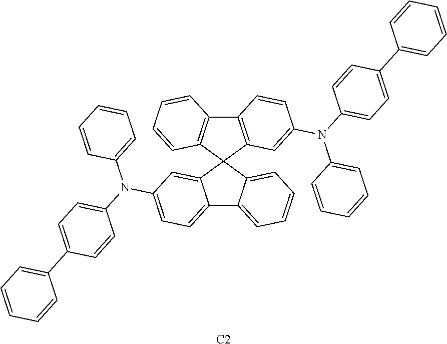 [SMILES: c1ccc(-c2ccc(N(c3ccccc3)c3ccc4c(c3)C3(c5ccccc5-4)c4ccccc4-c4ccc(N(c5ccccc5)c5ccc(-c6ccccc6)cc5)cc43)cc2)cc1 ; "C2"]

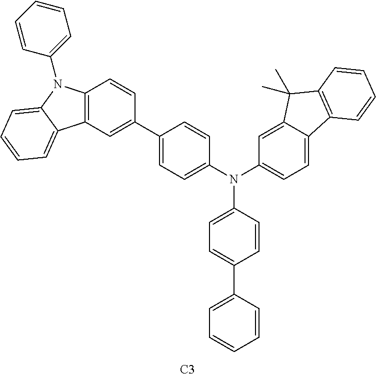 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; "C3"]

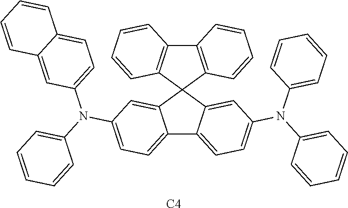 [SMILES: c1ccc(N(c2ccccc2)c2ccc3c(c2)C2(c4ccccc4-c4ccccc42)c2cc(N(c4ccccc4)c4ccc5ccccc5c4)ccc2-3)cc1 ; "C4"]

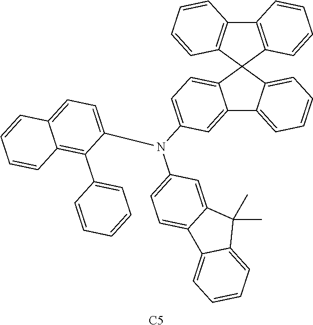 [SMILES: CC1(C)c2ccccc2-c2ccc(N(c3ccc4c(c3)-c3ccccc3C43c4ccccc4-c4ccccc43)c3ccc4ccccc4c3-c3ccccc3)cc21 ; "C5"]

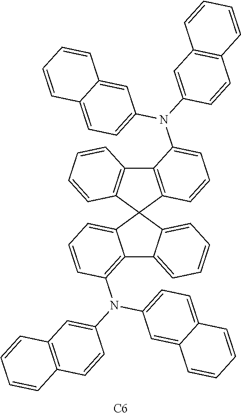

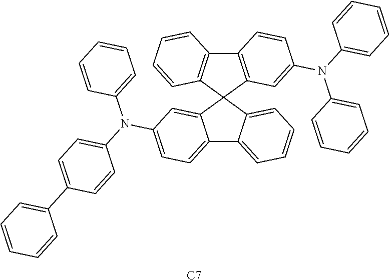 [SMILES: c1ccc(-c2ccc(N(c3ccccc3)c3ccc4c(c3)C3(c5ccccc5-c5ccc(N(c6ccccc6)c6ccccc6)cc53)c3ccccc3-4)cc2)cc1 ; "C7"]

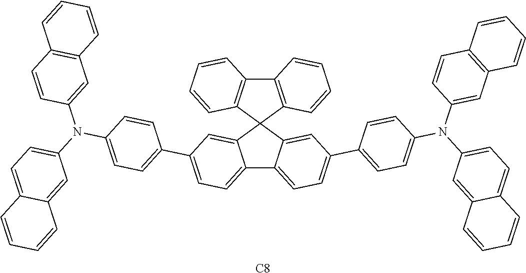 [SMILES: c1ccc2c(c1)-c1ccccc1C21c2cc(-c3ccc(N(c4ccc5ccccc5c4)c4ccc5ccccc5c4)cc3)ccc2-c2ccc(-c3ccc(N(c4ccc5ccccc5c4)c4ccc5ccccc5c4)cc3)cc21 ; "C8"]

Referring to Table 1, it was confirmed that the organic electroluminescent devices of Examples 1 to 27 had better driving voltage, efficiency, and lifespan, compared to the electroluminescent devices of Comparative Examples 1 to 8.

By including the amine-based compound represented by Formula 1, the light-emitting device may have excellent characteristics such as low driving voltage, high efficiency, high luminance, and long lifespan, and high-quality electronic apparatuses and electronic devices may be manufactured using the light-emitting device.

Embodiments have been disclosed herein, and although terms are employed, they are used and are to be interpreted in a generic and descriptive sense only and not for the purposes of limitation. In some instances, as would be apparent by one of ordinary skill in the art, features, characteristics, and/or elements described in connection with an embodiment may be used singly or in combination with features, characteristics, and/or elements described in connection with other embodiments unless otherwise specifically indicated. Accordingly, it will be understood by those of ordinary skill in the art that various changes in form and details may be made without departing from the spirit and scope of the disclosure as set forth in the claims.

What is claimed is:

1. A light-emitting device comprising:
a first electrode;
a second electrode facing the first electrode; and
an interlayer between the first electrode and the second electrode, wherein the interlayer comprises:
an emission layer; and
a hole transport region between the first electrode and the emission layer, and
the hole transport region comprises an amine-based compound represented by Formula 1: 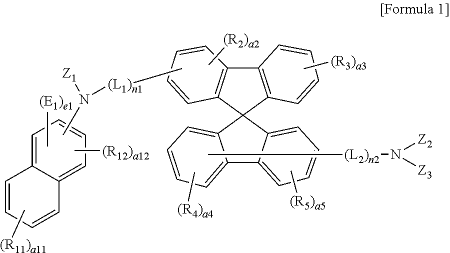
wherein in Formula 1,
L₁ and L₂ are each independently a single bond, a C₅-C₆₀ carbocyclic group unsubstituted or substituted with at least one R₁₀ₐ, or a C₁-C₆₀ heterocyclic group unsubstituted or substituted with at least one R₁₀ₐ,
n1 and n2 are each independently an integer from 1 to 3,
Z₁ to Z₃ and E₁ are each independently deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
e1 is an integer from 1 to 7,
R₁₁, R₁₂, and R₂ to R₅ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
a11 is an integer from 0 to 4,
a12 is an integer from 0 to 3,
a2 is an integer from 0 to 3,
a3 to a5 are each independently an integer from 0 to 4, R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or a combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a C₃-C₆₀ carbocyclic group, a C₁-C₆₀ heterocyclic group, or a combination thereof.

2. The light-emitting device of claim 1, wherein
the hole transport region comprises a hole injection layer, a hole transport layer, an emission auxiliary layer, an electron blocking layer, or a combination thereof, and
at least one of the hole transport layer and the hole injection layer comprises the amine-based compound.

3. The light-emitting device of claim 1, further comprising:
a second compound comprising at least one IT electron-deficient nitrogen-containing C₁-C₆₀ heterocyclic group, a third compound comprising a group represented by Formula 3, a fourth compound that is a delayed fluorescence compound, a fifth compound that is a transition metal-containing organometallic compound, or a combination thereof, wherein
the second compound, the third compound, the fourth compound, and the fifth compound are different from each other: 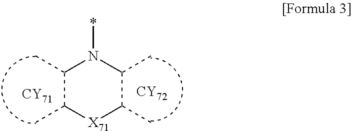
wherein in Formula 3,
ring CY₇₁ and ring CY₇₂ are each independently a π electron-rich C₃-C₆₀ cyclic group or a pyridine group,
X₇₁ is a single bond; or a linking group comprising O, S, N, B, C, Si, or a combination thereof, and
* indicates a binding site to an atom included in the remaining part other than Formula 3 in the third compound.

4. The light-emitting device of claim 3, wherein the second compound comprises a pyridine group, a pyrimidine group, a pyrazine group, a pyridazine group, a triazine group, or a combination thereof.

5. The light-emitting device of claim 3, wherein the fourth compound comprises at least one cyclic group comprising boron (B) and nitrogen (N) as ring-forming atoms.

6. The light-emitting device of claim 3, wherein
the fifth compound comprises a platinum atom and a tetradentate ligand bonded to the platinum atom, and
one of bonds between the platinum atom and the tetradentate ligand is a platinum-carbene bond.

7. The light-emitting device of claim 3, wherein
the fifth compound comprises an iridium atom and a tetradentate ligand bonded to the iridium atom, and
one of bonds between the iridium atom and the tetradentate ligand is an iridium-carbene bond.

8. The light-emitting device of claim 3, wherein
the emission layer comprises the second compound, the third compound, the fourth compound, the fifth compound, or a combination thereof, and
the emission layer emits blue light.

9. An electronic apparatus comprising the light-emitting device of claim 1.

10. The electronic apparatus of claim 9, further comprising a color filter, a color conversion layer, a touch screen layer, a polarizing layer, or a combination thereof.

11. The electronic apparatus of claim 9, further comprising:
a thin-film transistor, wherein
the thin-film transistor comprises a source electrode and a drain electrode, and
the first electrode of the light-emitting device is electrically connected to the source electrode or the drain electrode.

12. An electronic device comprising the light-emitting device of claim 1.

13. The electronic device of claim 12, wherein the electronic device is a flat panel display, a curved display, a computer monitor, a medical monitor, a television (TV), an advertisement board, an indoor light, an outdoor light, a signal light, a head-up display, a fully transparent display, a partially transparent display, a flexible display, a rollable display, a foldable display, a stretchable display, a laser printer, a telephone, a portable phone, a tablet personal computer, a phablet, a personal digital assistant (PDA), a wearable device, a laptop computer, a digital camera, a camcorder, a viewfinder, a microdisplay, a three-dimensional (3D) display, a virtual reality display, an augmented reality display, a vehicle, a video wall with multiple displays tiled together, a theater screen, a stadium screen, a phototherapy device, or a sign.

14. An amine-based compound represented by Formula 1: 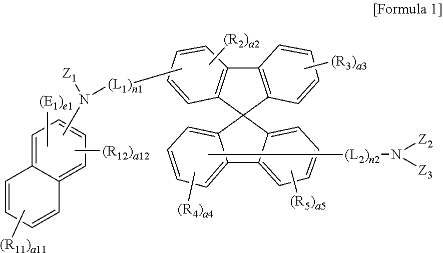
wherein in Formula 1,
L₁ and L₂ are each independently a single bond, a C₅-C₆₀ carbocyclic group unsubstituted or substituted with at least one R₁₀ₐ, or a C₁-C₆₀ heterocyclic group unsubstituted or substituted with at least one R₁₀ₐ,
n1 and n2 are each independently an integer from 1 to 3,
Z₁ to Z₃ and Et are each independently deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
e1 is an integer from 1 to 7,
R₁₁, R₁₂, and R₂ to R₅ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
a11 is an integer from 0 to 4,
a12 is an integer from 0 to 3,
a2 is an integer from 0 to 3,
a3 to a5 are each independently an integer from 0 to 4,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or a combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a C₃-C₆₀ carbocyclic group, a C₁-C₆₀ heterocyclic group, or a combination thereof.

15. The amine-based compound of claim 14, wherein Z₁ to Z₃ and E₁ are each independently a benzene group, a naphthalene group, a fluorene group, a carbazole group, a dibenzofuran group, a dibenzothiophene group, an azacarbazole group, an azadibenzofuran group, an azadibenzothiophene group, a pyridine group, a pyrimidine group, a pyrazine group, a quinoline group, or a quinoxaline group, each unsubstituted or substituted with at least one R₁₀ₐ.

16. The amine-based compound of claim 14, wherein Z₁ to Z₃ and E₁ are each independently a group represented by one of Formulae 1A-1 to 1A-16: 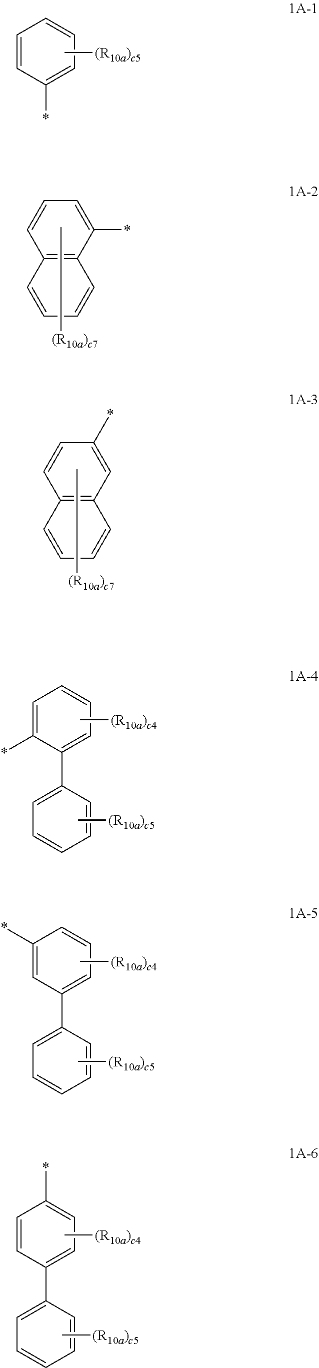 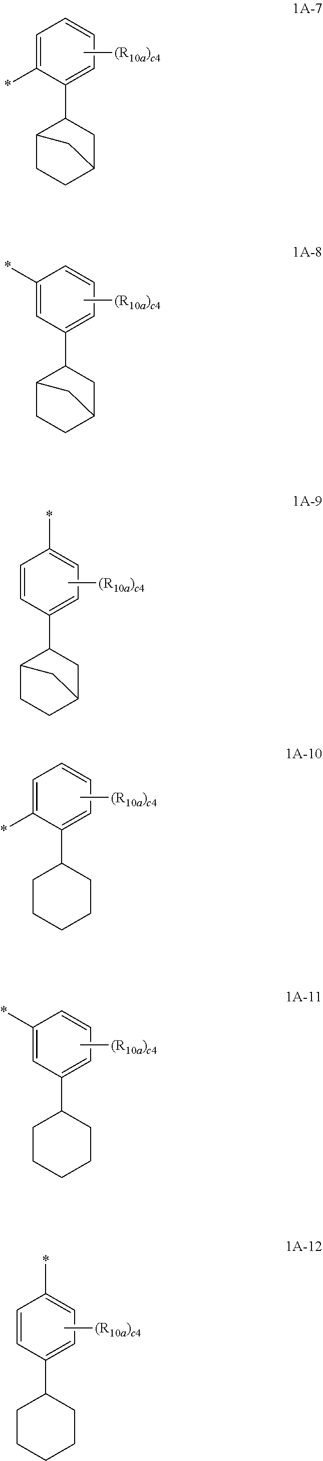 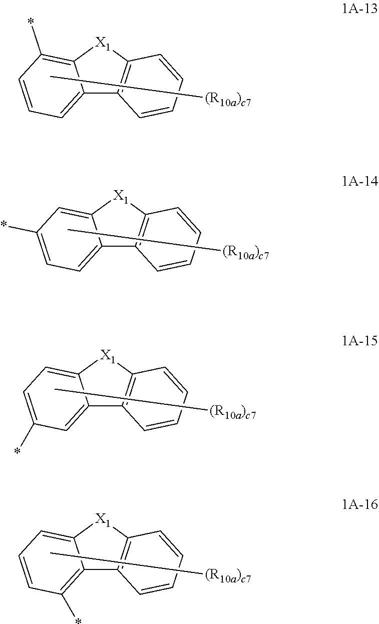
wherein in Formulae 1A-1 to 1A-16,
R₁₀ₐ is the same as defined in Formula 1,
c4 is an integer from 0 to 4,
c5 is an integer from 0 to 5,
c7 is an integer from 0 to 7,
X₁ is N(R₁), O, or S,
R₁ is the same as described in connection with R₁₁ in Formula 1, and
* indicates a binding site to N or a naphthalene group in Formula 1.

17. The amine-based compound of claim 14, wherein in Formula 1, a moiety represented by 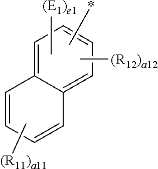
is a moiety represented by one of Formulae 1B-1 to 1B-14: 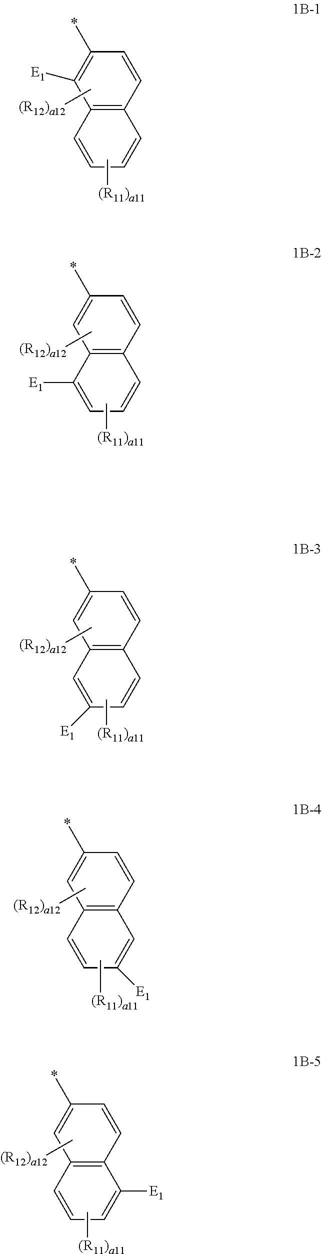 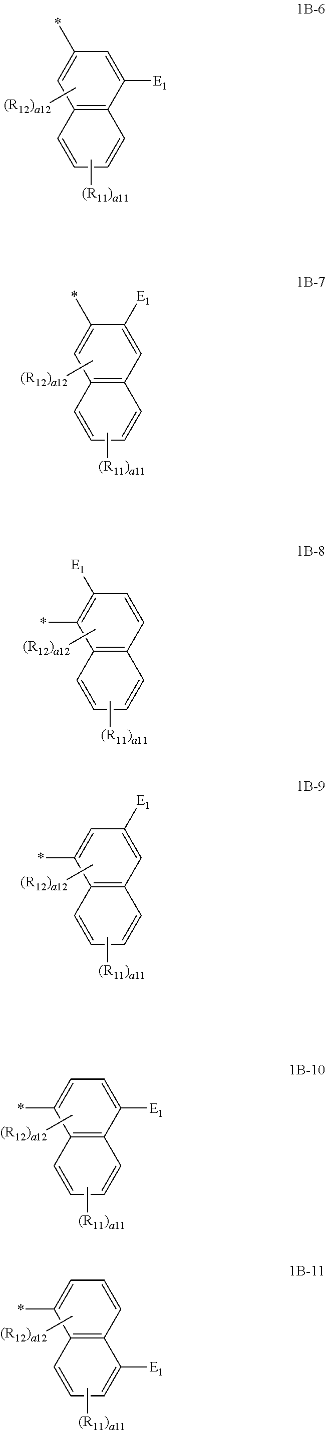 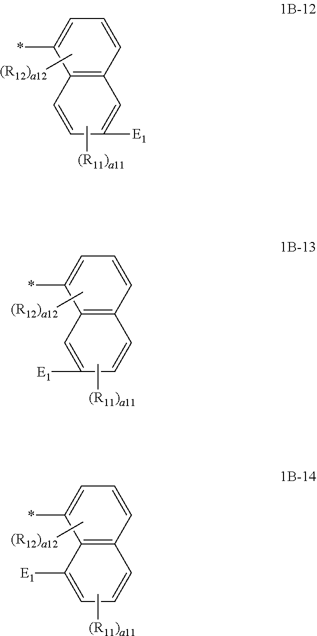
wherein in Formulae 1B-1 to 1B-14,
E₁, R₁₁, R₁₂, a11, and a12 are each the same as defined in Formula 1, and
* indicates a binding site to a neighboring N.

18. The amine-based compound of claim 14, wherein Z₂ and Z₃ are different from each other.

19. The amine-based compound of claim 14, wherein the amine-based compound is represented by Formula 1-1: 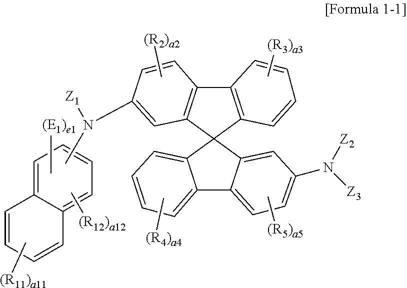
wherein in Formula 1-1,
a5 is an integer from 0 to 3, and
Z₁ to Z₃, E₁, e1, R₁₁, R₁₂, R₂ to R₅, a11, a12, and a2 to a4 are each the same as defined in Formula 1.

20. The amine-based compound of claim 14, wherein the amine-based compound is one of Compounds 1 to 206: 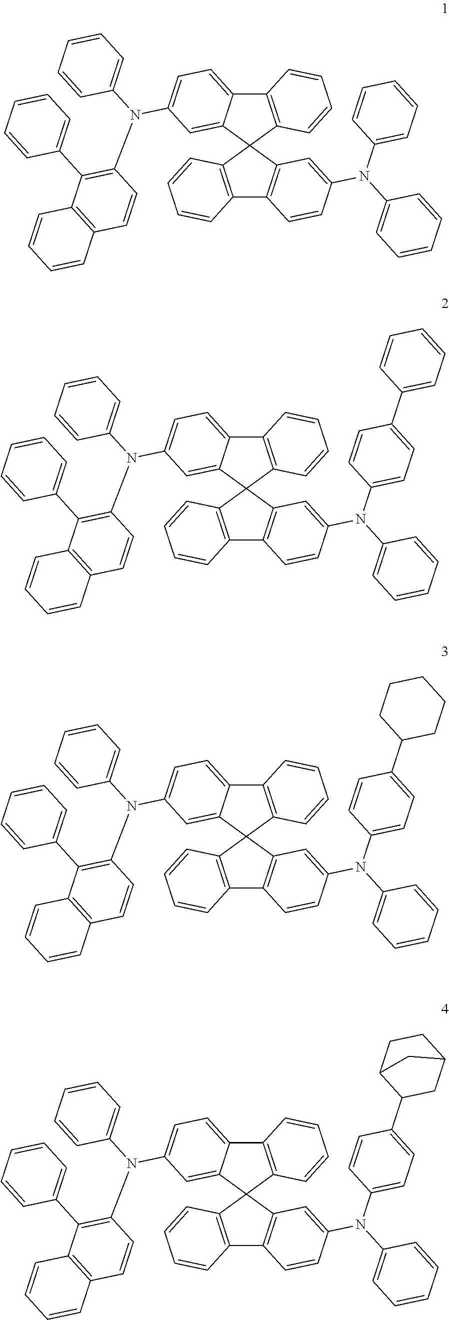 [SMILES: c1ccc(-c2c(N(c3ccccc3)c3ccc4c(c3)C3(c5ccccc5-c5ccc(N(c6ccccc6)c6ccccc6)cc53)c3ccccc3-4)ccc3ccccc23)cc1 ; c1ccc(-c2ccc(N(c3ccccc3)c3ccc4c(c3)C3(c5ccccc5-4)c4ccccc4-c4ccc(N(c5ccccc5)c5ccc6ccccc6c5-c5ccccc5)cc43)cc2)cc1 ; c1ccc(-c2c(N(c3ccccc3)c3ccc4c(c3)C3(c5ccccc5-c5ccc(N(c6ccccc6)c6ccc(C7CCCCC7)cc6)cc53)c3ccccc3-4)ccc3ccccc23)cc1 ; c1ccc(-c2c(N(c3ccccc3)c3ccc4c(c3)C3(c5ccccc5-c5ccc(N(c6ccccc6)c6ccc(C7CC8CCC7C8)cc6)cc53)c3ccccc3-4)ccc3ccccc23)cc1] 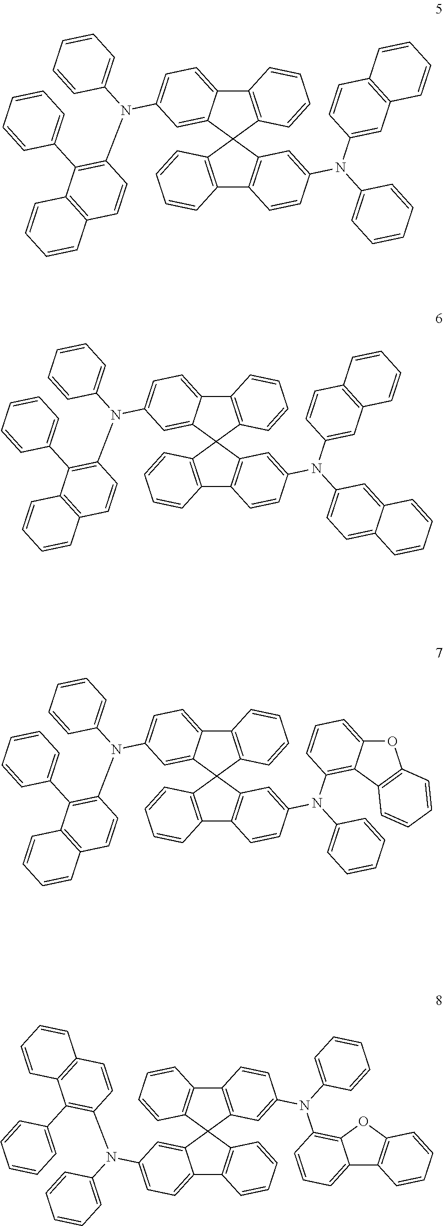 [SMILES: c1ccc(-c2c(N(c3ccccc3)c3ccc4c(c3)C3(c5ccccc5-c5ccc(N(c6ccccc6)c6ccc7ccccc7c6)cc53)c3ccccc3-4)ccc3ccccc23)cc1 ; c1ccc(-c2c(N(c3ccccc3)c3ccc4c(c3)C3(c5ccccc5-c5ccc(N(c6ccc7ccccc7c6)c6ccc7ccccc7c6)cc53)c3ccccc3-4)ccc3ccccc23)cc1 ; c1ccc(-c2c(N(c3ccccc3)c3ccc4c(c3)C3(c5ccccc5-4)c4ccccc4-c4ccc(N(c5ccccc5)c5cccc6oc7ccccc7c56)cc43)ccc3ccccc23)cc1 ; c1ccc(-c2c(N(c3ccccc3)c3ccc4c(c3)C3(c5ccccc5-4)c4ccccc4-c4ccc(N(c5ccccc5)c5cccc6c5oc5ccccc56)cc43)ccc3ccccc23)cc1] 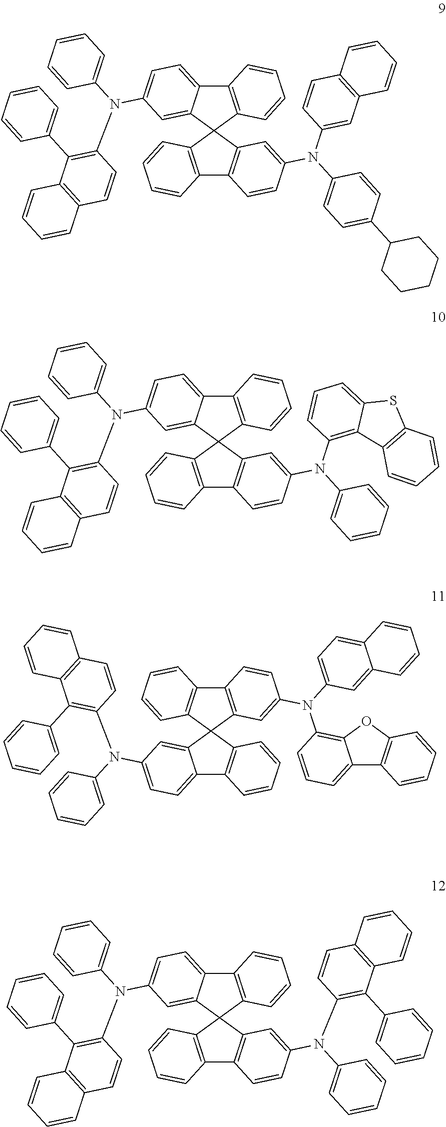 [SMILES: c1ccc(-c2c(N(c3ccccc3)c3ccc4c(c3)C3(c5ccccc5-c5ccc(N(c6ccc(C7CCCCC7)cc6)c6ccc7ccccc7c6)cc53)c3ccccc3-4)ccc3ccccc23)cc1 ; c1ccc(-c2c(N(c3ccccc3)c3ccc4c(c3)C3(c5ccccc5-4)c4ccccc4-c4ccc(N(c5ccccc5)c5cccc6sc7ccccc7c56)cc43)ccc3ccccc23)cc1 ; c1ccc(-c2c(N(c3ccccc3)c3ccc4c(c3)C3(c5ccccc5-4)c4ccccc4-c4ccc(N(c5ccc6ccccc6c5)c5cccc6c5oc5ccccc56)cc43)ccc3ccccc23)cc1 ; c1ccc(-c2c(N(c3ccccc3)c3ccc4c(c3)C3(c5ccccc5-4)c4ccccc4-c4ccc(N(c5ccccc5)c5ccc6ccccc6c5-c5ccccc5)cc43)ccc3ccccc23)cc1] 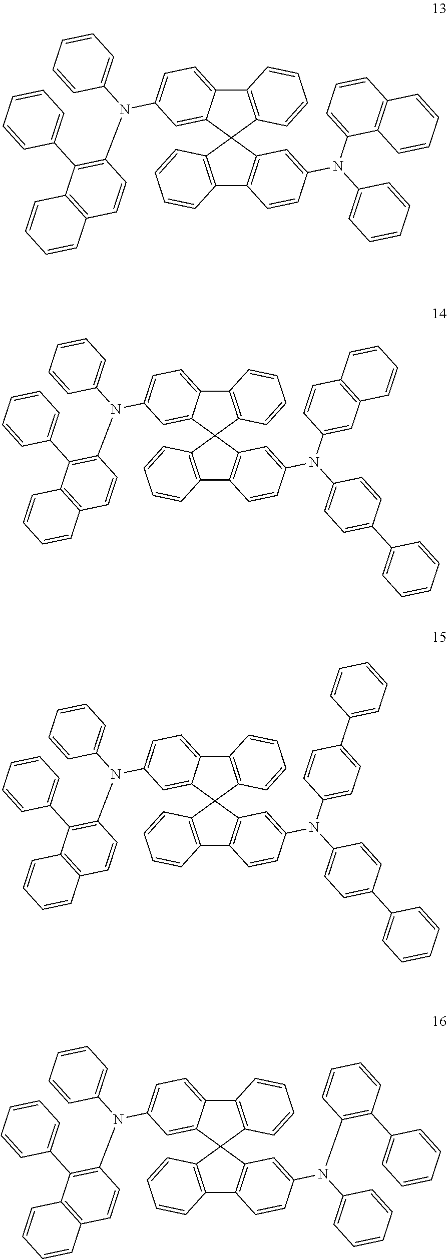 [SMILES: c1ccc(-c2c(N(c3ccccc3)c3ccc4c(c3)C3(c5ccccc5-4)c4ccccc4-c4ccc(N(c5ccccc5)c5cccc6ccccc56)cc43)ccc3ccccc23)cc1 ; c1ccc(-c2ccc(N(c3ccc4c(c3)C3(c5ccccc5-4)c4ccccc4-c4ccc(N(c5ccccc5)c5ccc6ccccc6c5-c5ccccc5)cc43)c3ccc4ccccc4c3)cc2)cc1 ; c1ccc(-c2ccc(N(c3ccc(-c4ccccc4)cc3)c3ccc4c(c3)C3(c5ccccc5-4)c4ccccc4-c4ccc(N(c5ccccc5)c5ccc6ccccc6c5-c5ccccc5)cc43)cc2)cc1 ; c1ccc(-c2ccccc2N(c2ccccc2)c2ccc3c(c2)C2(c4ccccc4-3)c3ccccc3-c3ccc(N(c4ccccc4)c4ccc5ccccc5c4-c4ccccc4)cc32)cc1] 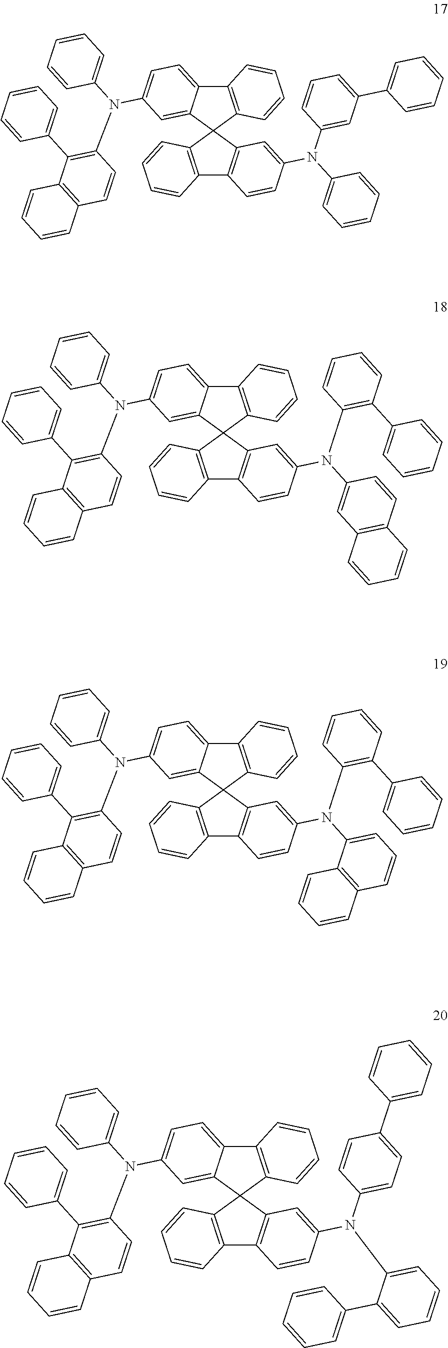 [SMILES: c1ccc(-c2cccc(N(c3ccccc3)c3ccc4c(c3)C3(c5ccccc5-4)c4ccccc4-c4ccc(N(c5ccccc5)c5ccc6ccccc6c5-c5ccccc5)cc43)c2)cc1 ; c1ccc(-c2ccccc2N(c2ccc3c(c2)C2(c4ccccc4-3)c3ccccc3-c3ccc(N(c4ccccc4)c4ccc5ccccc5c4-c4ccccc4)cc32)c2ccc3ccccc3c2)cc1 ; c1ccc(-c2ccccc2N(c2ccc3c(c2)C2(c4ccccc4-c4ccc(N(c5ccccc5)c5ccc6ccccc6c5-c5ccccc5)cc42)c2ccccc2-3)c2cccc3ccccc23)cc1 ; c1ccc(-c2ccc(N(c3ccc4c(c3)C3(c5ccccc5-4)c4ccccc4-c4ccc(N(c5ccccc5)c5ccc6ccccc6c5-c5ccccc5)cc43)c3ccccc3-c3ccccc3)cc2)cc1] 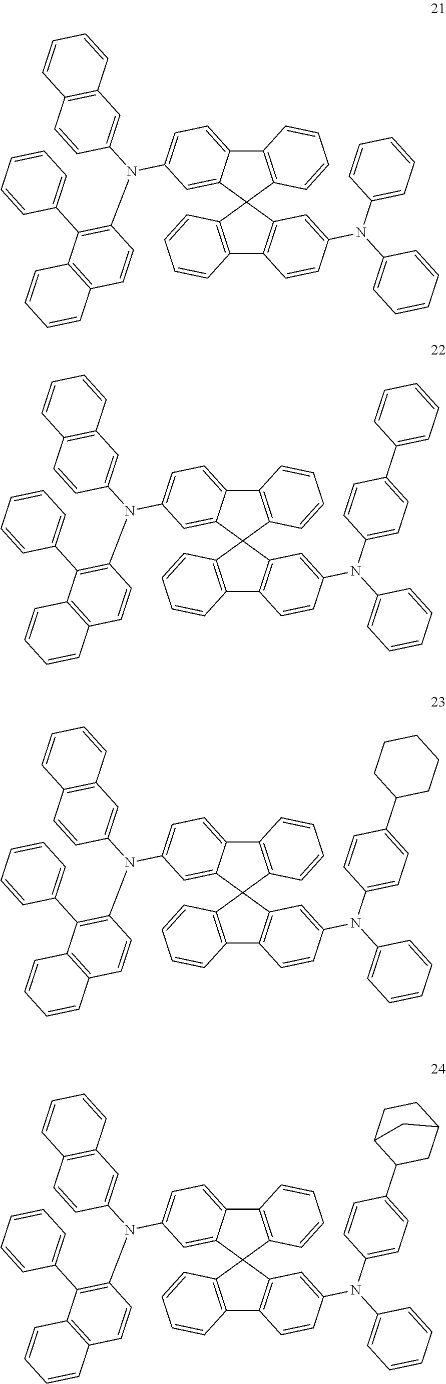 [SMILES: c1ccc(-c2c(N(c3ccc4c(c3)C3(c5ccccc5-c5ccc(N(c6ccccc6)c6ccccc6)cc53)c3ccccc3-4)c3ccc4ccccc4c3)ccc3ccccc23)cc1 ; c1ccc(-c2ccc(N(c3ccccc3)c3ccc4c(c3)C3(c5ccccc5-4)c4ccccc4-c4ccc(N(c5ccc6ccccc6c5)c5ccc6ccccc6c5-c5ccccc5)cc43)cc2)cc1 ; c1ccc(-c2c(N(c3ccc4c(c3)C3(c5ccccc5-c5ccc(N(c6ccccc6)c6ccc(C7CCCCC7)cc6)cc53)c3ccccc3-4)c3ccc4ccccc4c3)ccc3ccccc23)cc1 ; c1ccc(-c2c(N(c3ccc4c(c3)C3(c5ccccc5-c5ccc(N(c6ccccc6)c6ccc(C7CC8CCC7C8)cc6)cc53)c3ccccc3-4)c3ccc4ccccc4c3)ccc3ccccc23)cc1] 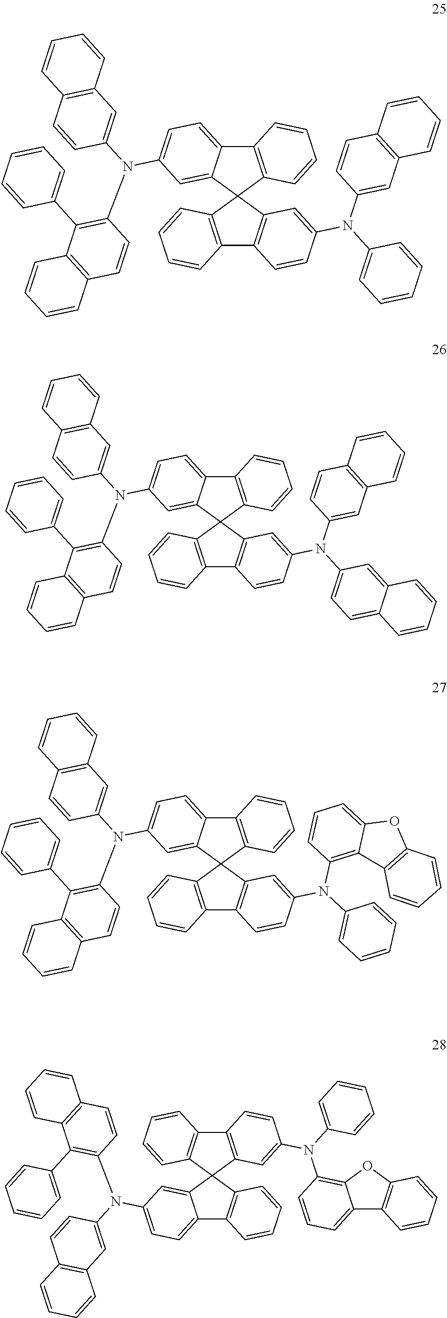 [SMILES: c1ccc(-c2c(N(c3ccc4c(c3)C3(c5ccccc5-c5ccc(N(c6ccccc6)c6ccc7ccccc7c6)cc53)c3ccccc3-4)c3ccc4ccccc4c3)ccc3ccccc23)cc1 ; c1ccc(-c2c(N(c3ccc4c(c3)C3(c5ccccc5-c5ccc(N(c6ccc7ccccc7c6)c6ccc7ccccc7c6)cc53)c3ccccc3-4)c3ccc4ccccc4c3)ccc3ccccc23)cc1 ; c1ccc(-c2c(N(c3ccc4c(c3)C3(c5ccccc5-4)c4ccccc4-c4ccc(N(c5ccccc5)c5cccc6oc7ccccc7c56)cc43)c3ccc4ccccc4c3)ccc3ccccc23)cc1 ; c1ccc(-c2c(N(c3ccc4c(c3)C3(c5ccccc5-4)c4ccccc4-c4ccc(N(c5ccccc5)c5cccc6c5oc5ccccc56)cc43)c3ccc4ccccc4c3)ccc3ccccc23)cc1] 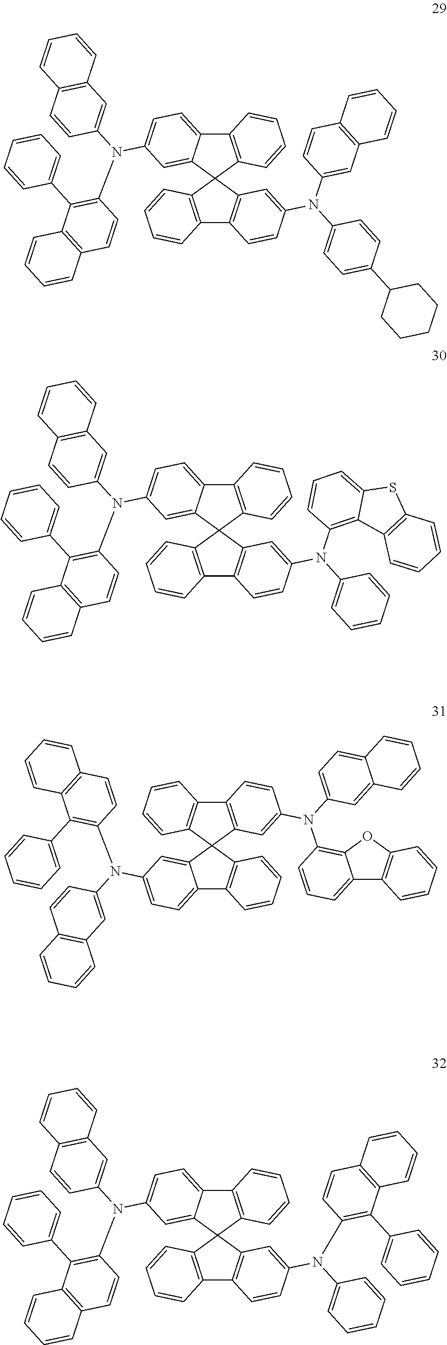 [SMILES: c1ccc(-c2c(N(c3ccc4c(c3)C3(c5ccccc5-c5ccc(N(c6ccc(C7CCCCC7)cc6)c6ccc7ccccc7c6)cc53)c3ccccc3-4)c3ccc4ccccc4c3)ccc3ccccc23)cc1 ; c1ccc(-c2c(N(c3ccc4c(c3)C3(c5ccccc5-4)c4ccccc4-c4ccc(N(c5ccccc5)c5cccc6sc7ccccc7c56)cc43)c3ccc4ccccc4c3)ccc3ccccc23)cc1 ; c1ccc(-c2c(N(c3ccc4c(c3)C3(c5ccccc5-4)c4ccccc4-c4ccc(N(c5ccc6ccccc6c5)c5cccc6c5oc5ccccc56)cc43)c3ccc4ccccc4c3)ccc3ccccc23)cc1 ; c1ccc(-c2c(N(c3ccccc3)c3ccc4c(c3)C3(c5ccccc5-4)c4ccccc4-c4ccc(N(c5ccc6ccccc6c5)c5ccc6ccccc6c5-c5ccccc5)cc43)ccc3ccccc23)cc1] 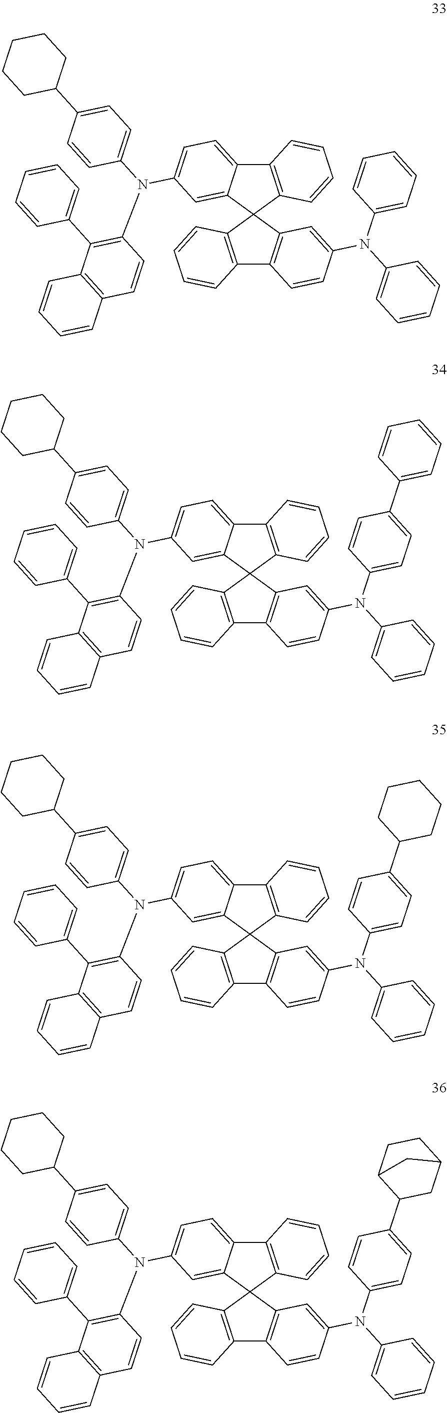 [SMILES: c1ccc(-c2c(N(c3ccc(C4CCCCC4)cc3)c3ccc4c(c3)C3(c5ccccc5-c5ccc(N(c6ccccc6)c6ccccc6)cc53)c3ccccc3-4)ccc3ccccc23)cc1 ; c1ccc(-c2ccc(N(c3ccccc3)c3ccc4c(c3)C3(c5ccccc5-4)c4ccccc4-c4ccc(N(c5ccc(C6CCCCC6)cc5)c5ccc6ccccc6c5-c5ccccc5)cc43)cc2)cc1 ; c1ccc(-c2c(N(c3ccc(C4CCCCC4)cc3)c3ccc4c(c3)C3(c5ccccc5-c5ccc(N(c6ccccc6)c6ccc(C7CCCCC7)cc6)cc53)c3ccccc3-4)ccc3ccccc23)cc1 ; c1ccc(-c2c(N(c3ccc(C4CCCCC4)cc3)c3ccc4c(c3)C3(c5ccccc5-c5ccc(N(c6ccccc6)c6ccc(C7CC8CCC7C8)cc6)cc53)c3ccccc3-4)ccc3ccccc23)cc1] 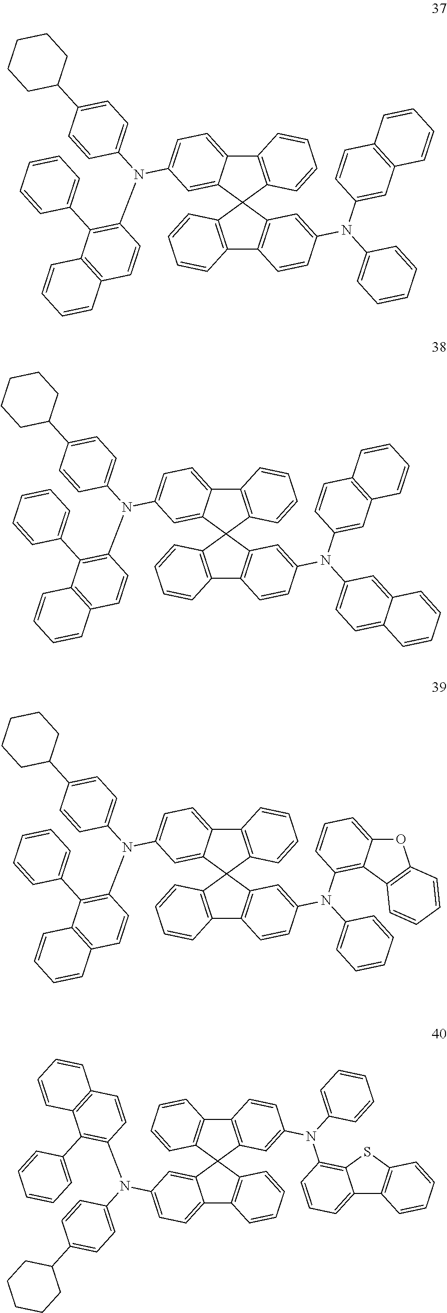 [SMILES: c1ccc(-c2c(N(c3ccc(C4CCCCC4)cc3)c3ccc4c(c3)C3(c5ccccc5-c5ccc(N(c6ccccc6)c6ccc7ccccc7c6)cc53)c3ccccc3-4)ccc3ccccc23)cc1 ; c1ccc(-c2c(N(c3ccc(C4CCCCC4)cc3)c3ccc4c(c3)C3(c5ccccc5-c5ccc(N(c6ccc7ccccc7c6)c6ccc7ccccc7c6)cc53)c3ccccc3-4)ccc3ccccc23)cc1 ; c1ccc(-c2c(N(c3ccc(C4CCCCC4)cc3)c3ccc4c(c3)C3(c5ccccc5-4)c4ccccc4-c4ccc(N(c5ccccc5)c5cccc6oc7ccccc7c56)cc43)ccc3ccccc23)cc1 ; c1ccc(-c2c(N(c3ccc(C4CCCCC4)cc3)c3ccc4c(c3)C3(c5ccccc5-4)c4ccccc4-c4ccc(N(c5ccccc5)c5cccc6c5sc5ccccc56)cc43)ccc3ccccc23)cc1] 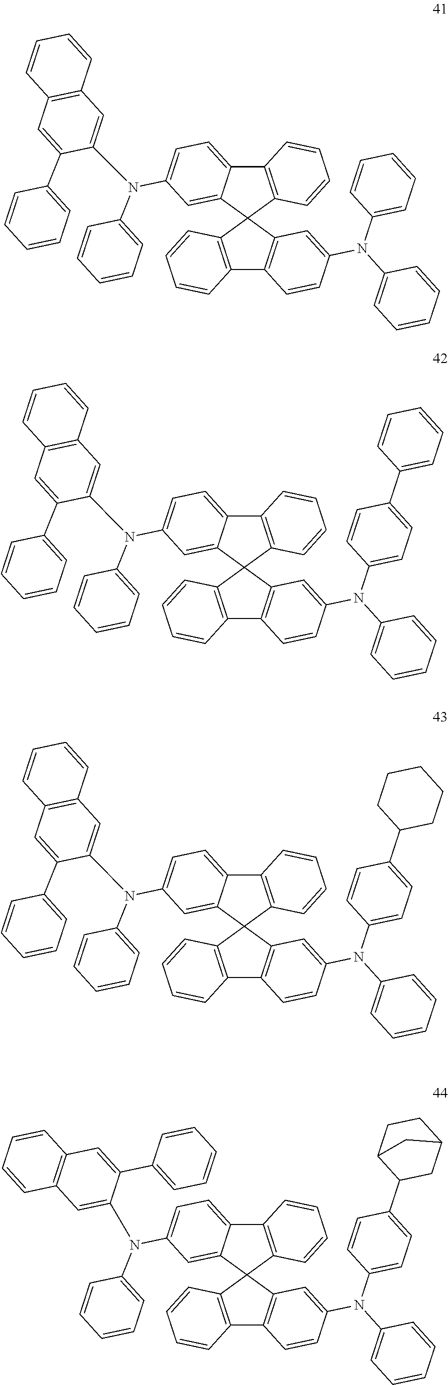 [SMILES: c1ccc(-c2cc3ccccc3cc2N(c2ccccc2)c2ccc3c(c2)C2(c4ccccc4-c4ccc(N(c5ccccc5)c5ccccc5)cc42)c2ccccc2-3)cc1 ; c1ccc(-c2ccc(N(c3ccccc3)c3ccc4c(c3)C3(c5ccccc5-4)c4ccccc4-c4ccc(N(c5ccccc5)c5cc6ccccc6cc5-c5ccccc5)cc43)cc2)cc1 ; c1ccc(-c2cc3ccccc3cc2N(c2ccccc2)c2ccc3c(c2)C2(c4ccccc4-c4ccc(N(c5ccccc5)c5ccc(C6CCCCC6)cc5)cc42)c2ccccc2-3)cc1 ; c1ccc(-c2cc3ccccc3cc2N(c2ccccc2)c2ccc3c(c2)C2(c4ccccc4-c4ccc(N(c5ccccc5)c5ccc(C6CC7CCC6C7)cc5)cc42)c2ccccc2-3)cc1] 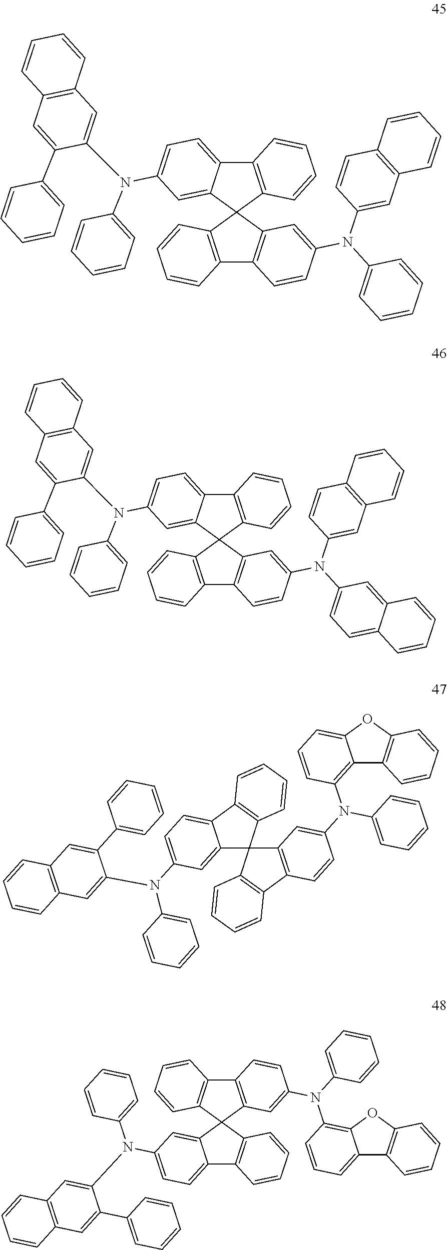 [SMILES: c1ccc(-c2cc3ccccc3cc2N(c2ccccc2)c2ccc3c(c2)C2(c4ccccc4-c4ccc(N(c5ccccc5)c5ccc6ccccc6c5)cc42)c2ccccc2-3)cc1 ; c1ccc(-c2cc3ccccc3cc2N(c2ccccc2)c2ccc3c(c2)C2(c4ccccc4-c4ccc(N(c5ccc6ccccc6c5)c5ccc6ccccc6c5)cc42)c2ccccc2-3)cc1 ; c1ccc(-c2cc3ccccc3cc2N(c2ccccc2)c2ccc3c(c2)C2(c4ccccc4-3)c3ccccc3-c3ccc(N(c4ccccc4)c4cccc5oc6ccccc6c45)cc32)cc1 ; c1ccc(-c2cc3ccccc3cc2N(c2ccccc2)c2ccc3c(c2)C2(c4ccccc4-3)c3ccccc3-c3ccc(N(c4ccccc4)c4cccc5c4oc4ccccc45)cc32)cc1] 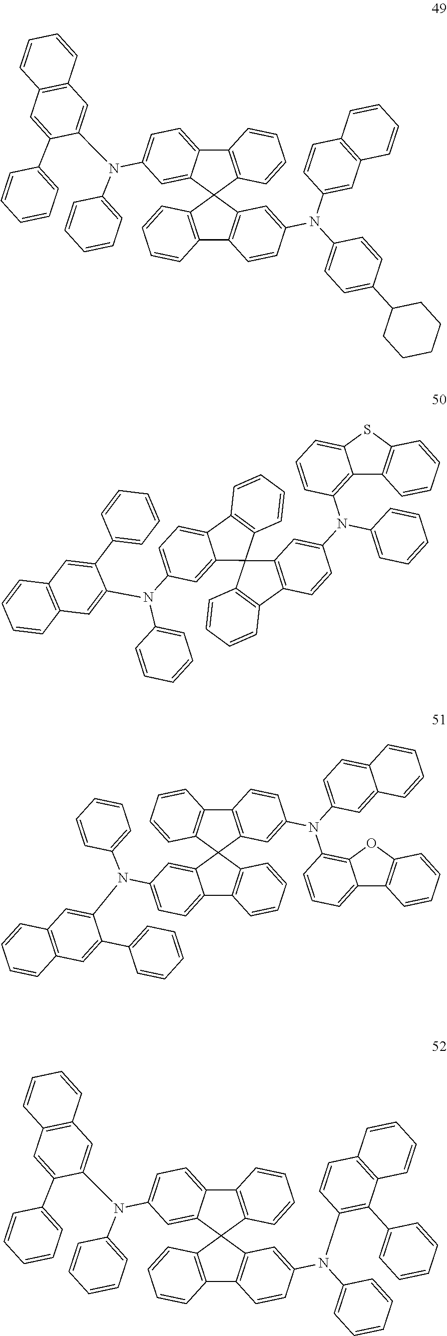 [SMILES: c1ccc(-c2cc3ccccc3cc2N(c2ccccc2)c2ccc3c(c2)C2(c4ccccc4-c4ccc(N(c5ccc(C6CCCCC6)cc5)c5ccc6ccccc6c5)cc42)c2ccccc2-3)cc1 ; c1ccc(-c2cc3ccccc3cc2N(c2ccccc2)c2ccc3c(c2)C2(c4ccccc4-3)c3ccccc3-c3ccc(N(c4ccccc4)c4cccc5sc6ccccc6c45)cc32)cc1 ; c1ccc(-c2cc3ccccc3cc2N(c2ccccc2)c2ccc3c(c2)C2(c4ccccc4-3)c3ccccc3-c3ccc(N(c4ccc5ccccc5c4)c4cccc5c4oc4ccccc45)cc32)cc1 ; c1ccc(-c2cc3ccccc3cc2N(c2ccccc2)c2ccc3c(c2)C2(c4ccccc4-3)c3ccccc3-c3ccc(N(c4ccccc4)c4ccc5ccccc5c4-c4ccccc4)cc32)cc1] 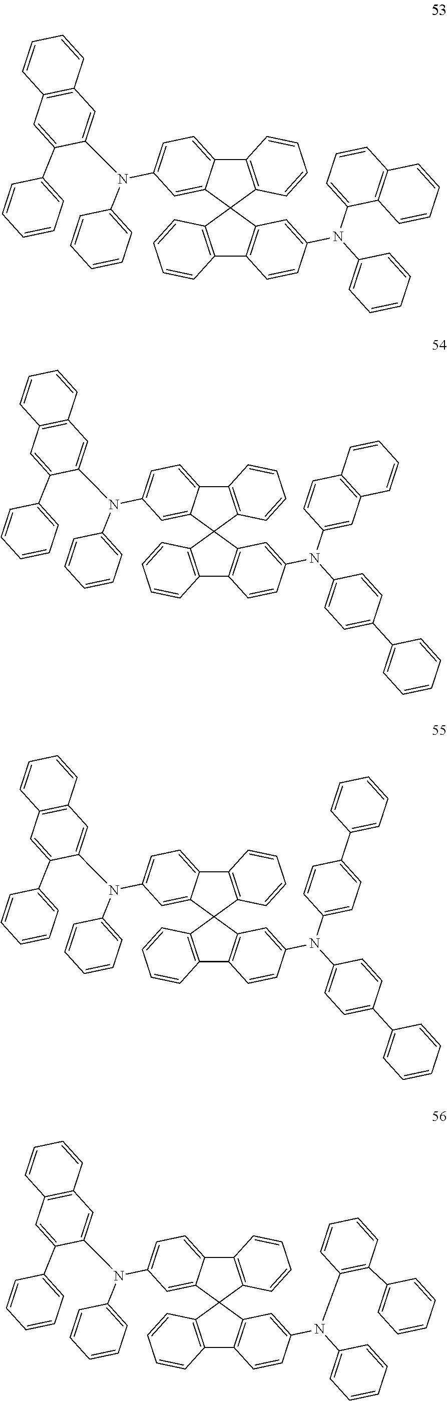 [SMILES: c1ccc(-c2cc3ccccc3cc2N(c2ccccc2)c2ccc3c(c2)C2(c4ccccc4-3)c3ccccc3-c3ccc(N(c4ccccc4)c4cccc5ccccc45)cc32)cc1 ; c1ccc(-c2ccc(N(c3ccc4c(c3)C3(c5ccccc5-4)c4ccccc4-c4ccc(N(c5ccccc5)c5cc6ccccc6cc5-c5ccccc5)cc43)c3ccc4ccccc4c3)cc2)cc1 ; c1ccc(-c2ccc(N(c3ccc(-c4ccccc4)cc3)c3ccc4c(c3)C3(c5ccccc5-4)c4ccccc4-c4ccc(N(c5ccccc5)c5cc6ccccc6cc5-c5ccccc5)cc43)cc2)cc1 ; c1ccc(-c2ccccc2N(c2ccccc2)c2ccc3c(c2)C2(c4ccccc4-3)c3ccccc3-c3ccc(N(c4ccccc4)c4cc5ccccc5cc4-c4ccccc4)cc32)cc1] 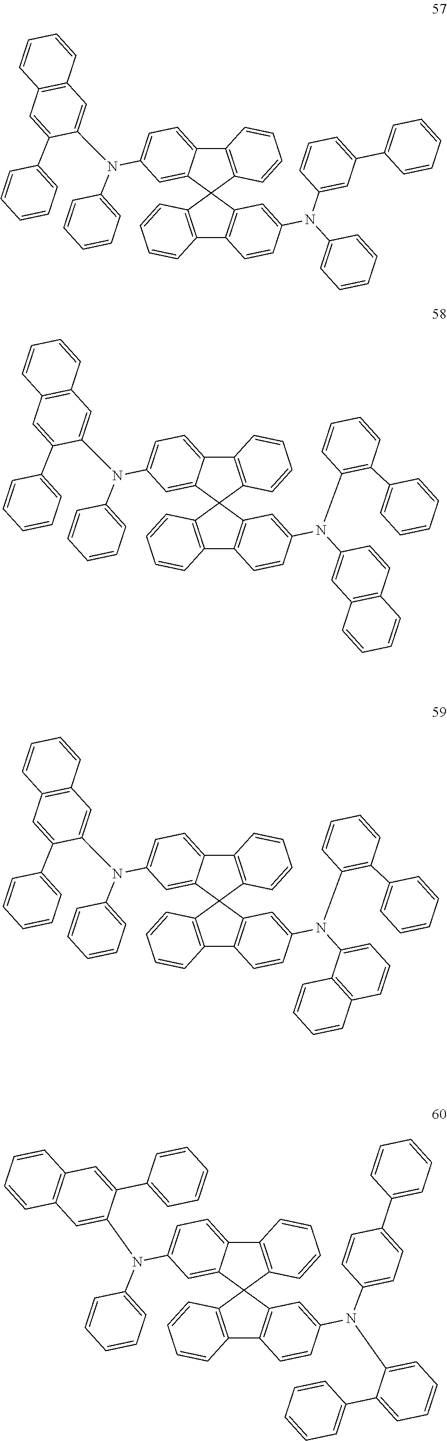 [SMILES: c1ccc(-c2cccc(N(c3ccccc3)c3ccc4c(c3)C3(c5ccccc5-4)c4ccccc4-c4ccc(N(c5ccccc5)c5cc6ccccc6cc5-c5ccccc5)cc43)c2)cc1 ; c1ccc(-c2ccccc2N(c2ccc3c(c2)C2(c4ccccc4-3)c3ccccc3-c3ccc(N(c4ccccc4)c4cc5ccccc5cc4-c4ccccc4)cc32)c2ccc3ccccc3c2)cc1 ; c1ccc(-c2cc3ccccc3cc2N(c2ccccc2)c2ccc3c(c2)C2(c4ccccc4-3)c3ccccc3-c3ccc(N(c4ccccc4-c4ccccc4)c4cccc5ccccc45)cc32)cc1 ; c1ccc(-c2ccc(N(c3ccc4c(c3)C3(c5ccccc5-4)c4ccccc4-c4ccc(N(c5ccccc5)c5cc6ccccc6cc5-c5ccccc5)cc43)c3ccccc3-c3ccccc3)cc2)cc1] 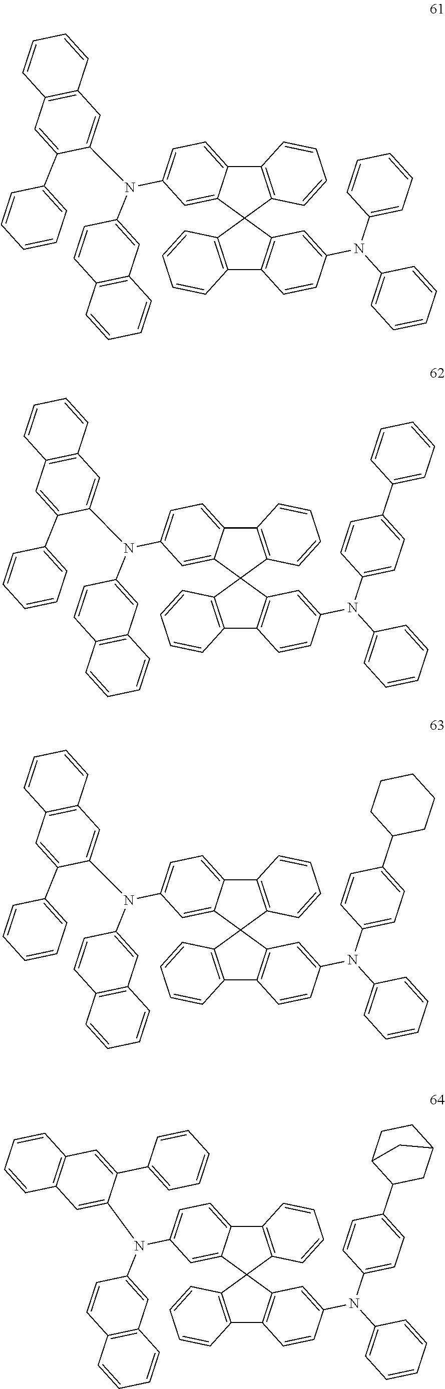 [SMILES: c1ccc(-c2cc3ccccc3cc2N(c2ccc3c(c2)C2(c4ccccc4-c4ccc(N(c5ccccc5)c5ccccc5)cc42)c2ccccc2-3)c2ccc3ccccc3c2)cc1 ; c1ccc(-c2ccc(N(c3ccccc3)c3ccc4c(c3)C3(c5ccccc5-4)c4ccccc4-c4ccc(N(c5ccc6ccccc6c5)c5cc6ccccc6cc5-c5ccccc5)cc43)cc2)cc1 ; c1ccc(-c2cc3ccccc3cc2N(c2ccc3c(c2)C2(c4ccccc4-c4ccc(N(c5ccccc5)c5ccc(C6CCCCC6)cc5)cc42)c2ccccc2-3)c2ccc3ccccc3c2)cc1 ; c1ccc(-c2cc3ccccc3cc2N(c2ccc3c(c2)C2(c4ccccc4-c4ccc(N(c5ccccc5)c5ccc(C6CC7CCC6C7)cc5)cc42)c2ccccc2-3)c2ccc3ccccc3c2)cc1] 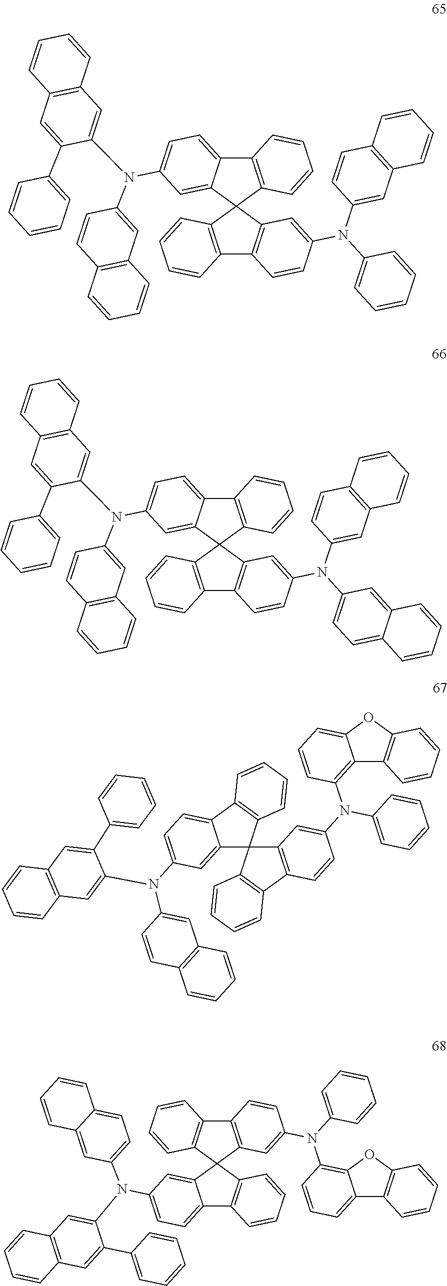 [SMILES: c1ccc(-c2cc3ccccc3cc2N(c2ccc3c(c2)C2(c4ccccc4-c4ccc(N(c5ccccc5)c5ccc6ccccc6c5)cc42)c2ccccc2-3)c2ccc3ccccc3c2)cc1 ; c1ccc(-c2cc3ccccc3cc2N(c2ccc3c(c2)C2(c4ccccc4-c4ccc(N(c5ccc6ccccc6c5)c5ccc6ccccc6c5)cc42)c2ccccc2-3)c2ccc3ccccc3c2)cc1 ; c1ccc(-c2cc3ccccc3cc2N(c2ccc3c(c2)C2(c4ccccc4-3)c3ccccc3-c3ccc(N(c4ccccc4)c4cccc5oc6ccccc6c45)cc32)c2ccc3ccccc3c2)cc1 ; c1ccc(-c2cc3ccccc3cc2N(c2ccc3c(c2)C2(c4ccccc4-3)c3ccccc3-c3ccc(N(c4ccccc4)c4cccc5c4oc4ccccc45)cc32)c2ccc3ccccc3c2)cc1] 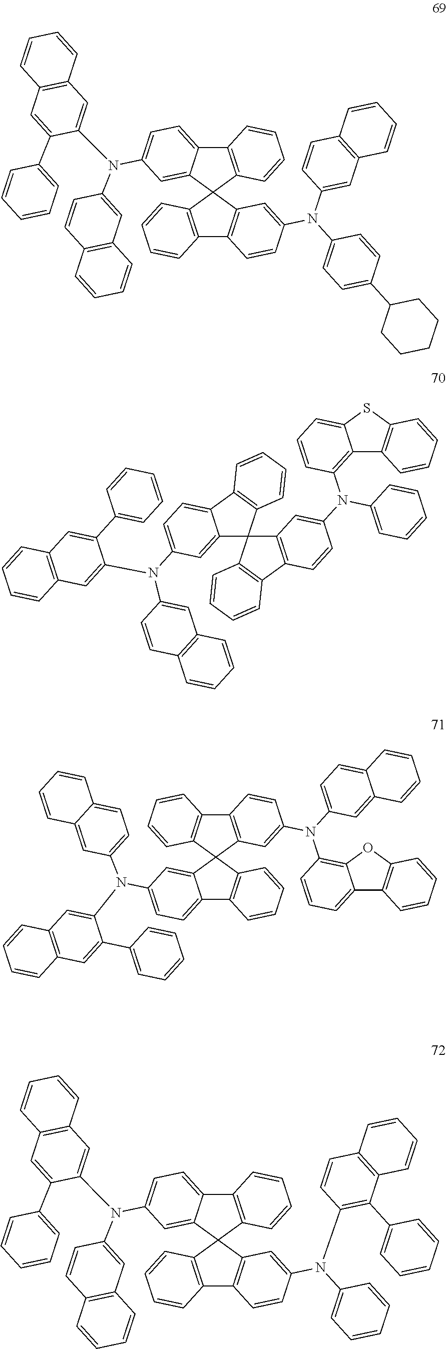 [SMILES: c1ccc(-c2cc3ccccc3cc2N(c2ccc3c(c2)C2(c4ccccc4-c4ccc(N(c5ccc(C6CCCCC6)cc5)c5ccc6ccccc6c5)cc42)c2ccccc2-3)c2ccc3ccccc3c2)cc1 ; c1ccc(-c2cc3ccccc3cc2N(c2ccc3c(c2)C2(c4ccccc4-3)c3ccccc3-c3ccc(N(c4ccccc4)c4cccc5sc6ccccc6c45)cc32)c2ccc3ccccc3c2)cc1 ; c1ccc(-c2cc3ccccc3cc2N(c2ccc3c(c2)C2(c4ccccc4-3)c3ccccc3-c3ccc(N(c4ccc5ccccc5c4)c4cccc5c4oc4ccccc45)cc32)c2ccc3ccccc3c2)cc1 ; c1ccc(-c2cc3ccccc3cc2N(c2ccc3c(c2)C2(c4ccccc4-3)c3ccccc3-c3ccc(N(c4ccccc4)c4ccc5ccccc5c4-c4ccccc4)cc32)c2ccc3ccccc3c2)cc1] 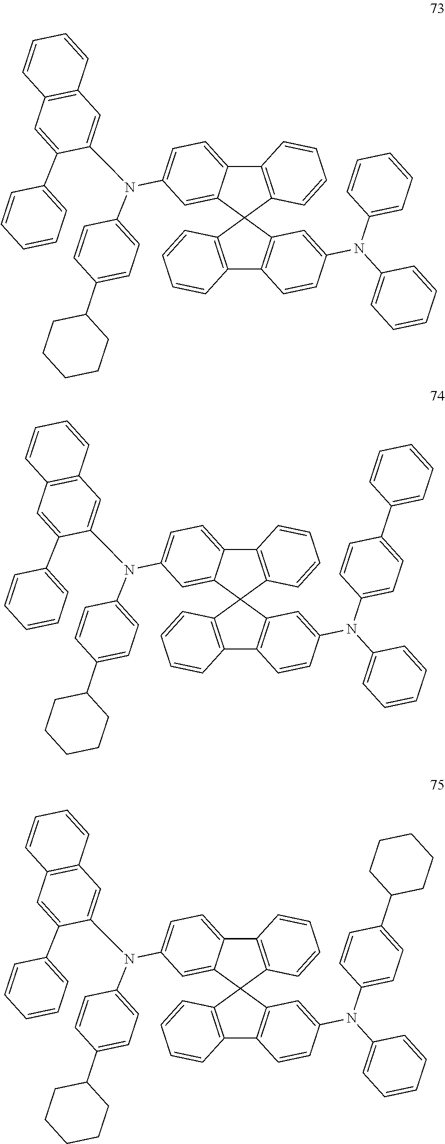 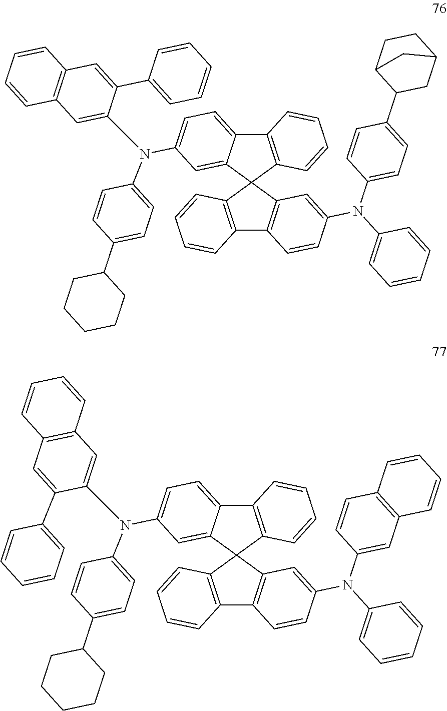 [SMILES: c1ccc(-c2cc3ccccc3cc2N(c2ccc(C3CCCCC3)cc2)c2ccc3c(c2)C2(c4ccccc4-c4ccc(N(c5ccccc5)c5ccc(C6CC7CCC6C7)cc5)cc42)c2ccccc2-3)cc1 ; c1ccc(-c2cc3ccccc3cc2N(c2ccc(C3CCCCC3)cc2)c2ccc3c(c2)C2(c4ccccc4-c4ccc(N(c5ccccc5)c5ccc6ccccc6c5)cc42)c2ccccc2-3)cc1] 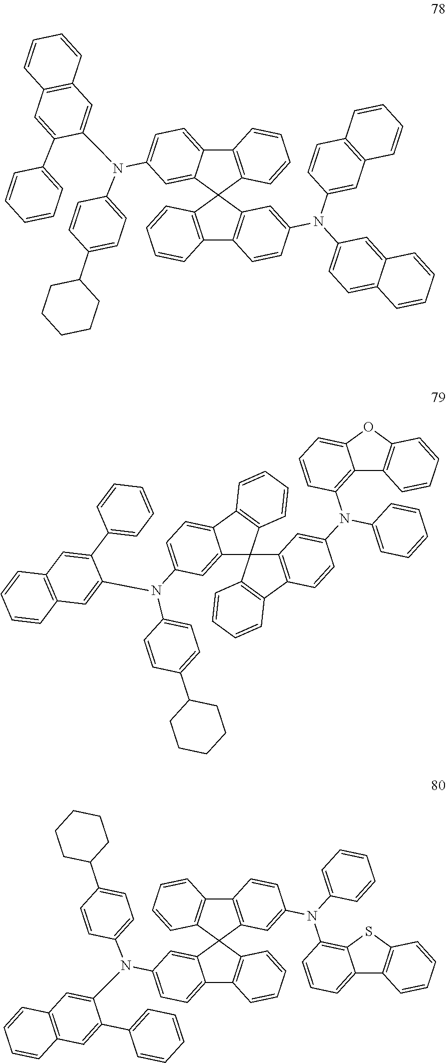 [SMILES: c1ccc(-c2cc3ccccc3cc2N(c2ccc(C3CCCCC3)cc2)c2ccc3c(c2)C2(c4ccccc4-c4ccc(N(c5ccc6ccccc6c5)c5ccc6ccccc6c5)cc42)c2ccccc2-3)cc1 ; c1ccc(-c2cc3ccccc3cc2N(c2ccc(C3CCCCC3)cc2)c2ccc3c(c2)C2(c4ccccc4-3)c3ccccc3-c3ccc(N(c4ccccc4)c4cccc5oc6ccccc6c45)cc32)cc1 ; c1ccc(-c2cc3ccccc3cc2N(c2ccc(C3CCCCC3)cc2)c2ccc3c(c2)C2(c4ccccc4-3)c3ccccc3-c3ccc(N(c4ccccc4)c4cccc5c4sc4ccccc45)cc32)cc1] 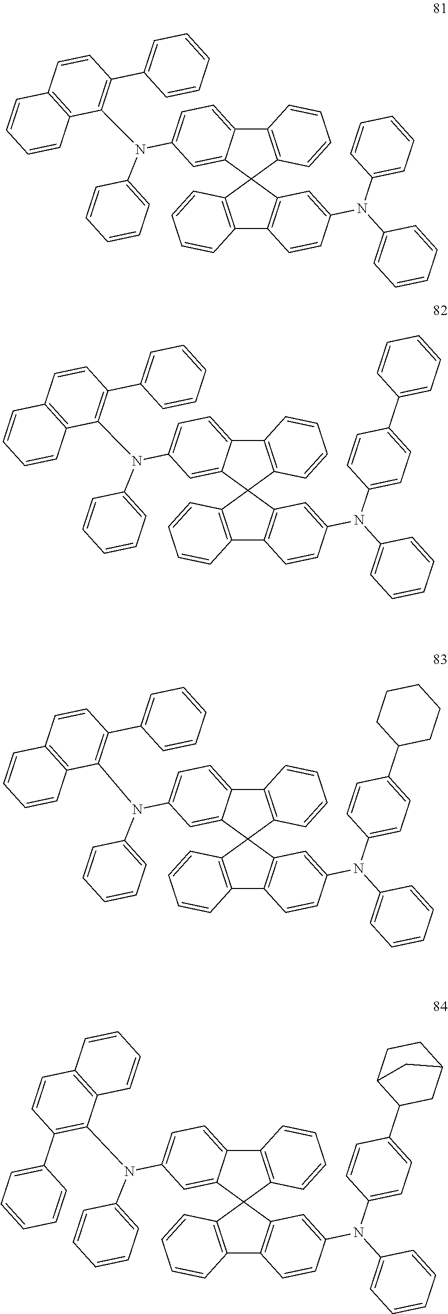 [SMILES: c1ccc(-c2ccc3ccccc3c2N(c2ccccc2)c2ccc3c(c2)C2(c4ccccc4-c4ccc(N(c5ccccc5)c5ccccc5)cc42)c2ccccc2-3)cc1 ; c1ccc(-c2ccc(N(c3ccccc3)c3ccc4c(c3)C3(c5ccccc5-4)c4ccccc4-c4ccc(N(c5ccccc5)c5c(-c6ccccc6)ccc6ccccc56)cc43)cc2)cc1 ; c1ccc(-c2ccc3ccccc3c2N(c2ccccc2)c2ccc3c(c2)C2(c4ccccc4-c4ccc(N(c5ccccc5)c5ccc(C6CCCCC6)cc5)cc42)c2ccccc2-3)cc1 ; c1ccc(-c2ccc3ccccc3c2N(c2ccccc2)c2ccc3c(c2)C2(c4ccccc4-c4ccc(N(c5ccccc5)c5ccc(C6CC7CCC6C7)cc5)cc42)c2ccccc2-3)cc1] 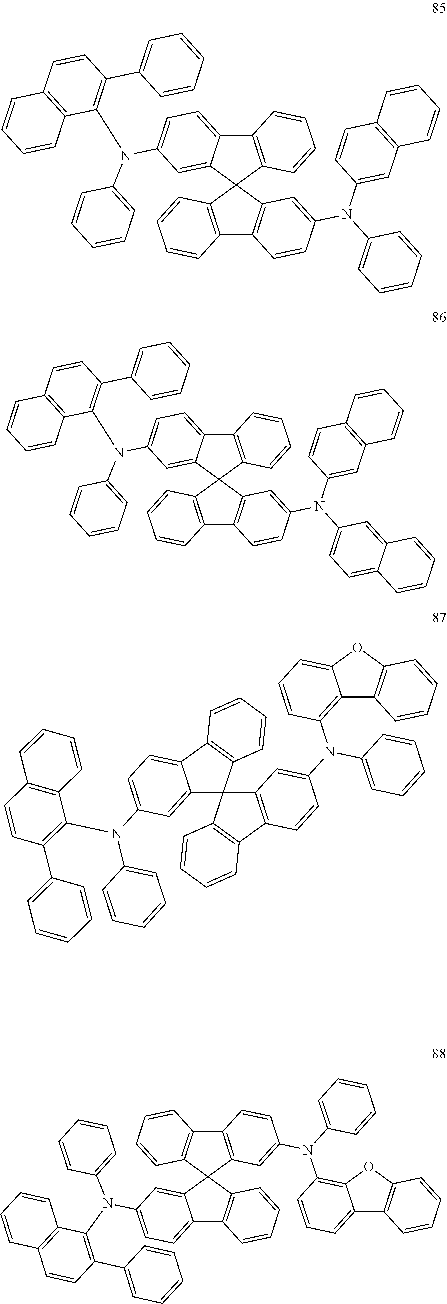 [SMILES: c1ccc(-c2ccc3ccccc3c2N(c2ccccc2)c2ccc3c(c2)C2(c4ccccc4-c4ccc(N(c5ccccc5)c5ccc6ccccc6c5)cc42)c2ccccc2-3)cc1 ; c1ccc(-c2ccc3ccccc3c2N(c2ccccc2)c2ccc3c(c2)C2(c4ccccc4-c4ccc(N(c5ccc6ccccc6c5)c5ccc6ccccc6c5)cc42)c2ccccc2-3)cc1 ; c1ccc(-c2ccc3ccccc3c2N(c2ccccc2)c2ccc3c(c2)C2(c4ccccc4-3)c3ccccc3-c3ccc(N(c4ccccc4)c4cccc5oc6ccccc6c45)cc32)cc1 ; c1ccc(-c2ccc3ccccc3c2N(c2ccccc2)c2ccc3c(c2)C2(c4ccccc4-3)c3ccccc3-c3ccc(N(c4ccccc4)c4cccc5c4oc4ccccc45)cc32)cc1] 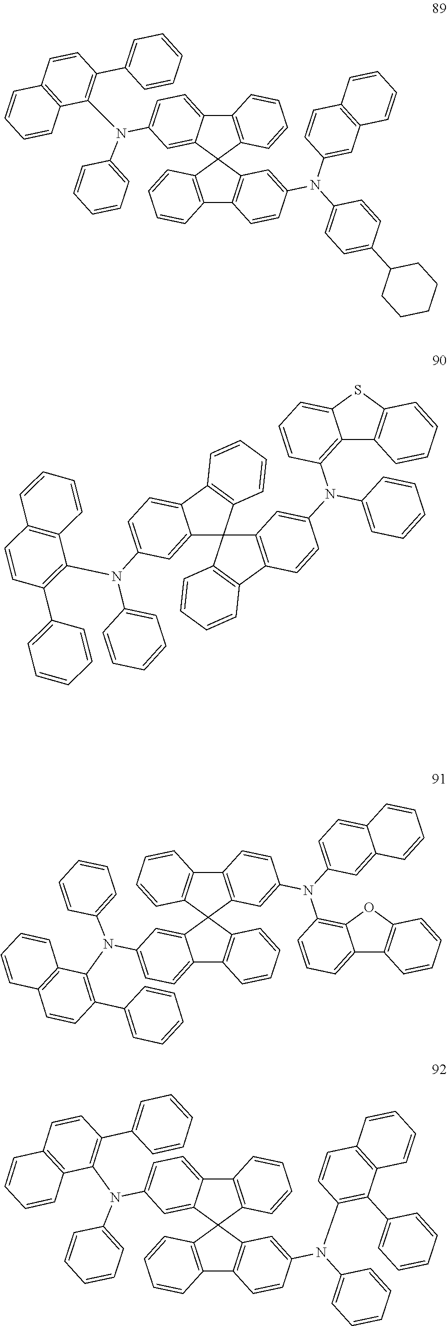 [SMILES: c1ccc(-c2ccc3ccccc3c2N(c2ccccc2)c2ccc3c(c2)C2(c4ccccc4-c4ccc(N(c5ccc(C6CCCCC6)cc5)c5ccc6ccccc6c5)cc42)c2ccccc2-3)cc1 ; c1ccc(-c2ccc3ccccc3c2N(c2ccccc2)c2ccc3c(c2)C2(c4ccccc4-3)c3ccccc3-c3ccc(N(c4ccccc4)c4cccc5sc6ccccc6c45)cc32)cc1 ; c1ccc(-c2ccc3ccccc3c2N(c2ccccc2)c2ccc3c(c2)C2(c4ccccc4-3)c3ccccc3-c3ccc(N(c4ccc5ccccc5c4)c4cccc5c4oc4ccccc45)cc32)cc1 ; c1ccc(-c2ccc3ccccc3c2N(c2ccccc2)c2ccc3c(c2)C2(c4ccccc4-c4ccc(N(c5ccccc5)c5ccc6ccccc6c5-c5ccccc5)cc42)c2ccccc2-3)cc1] 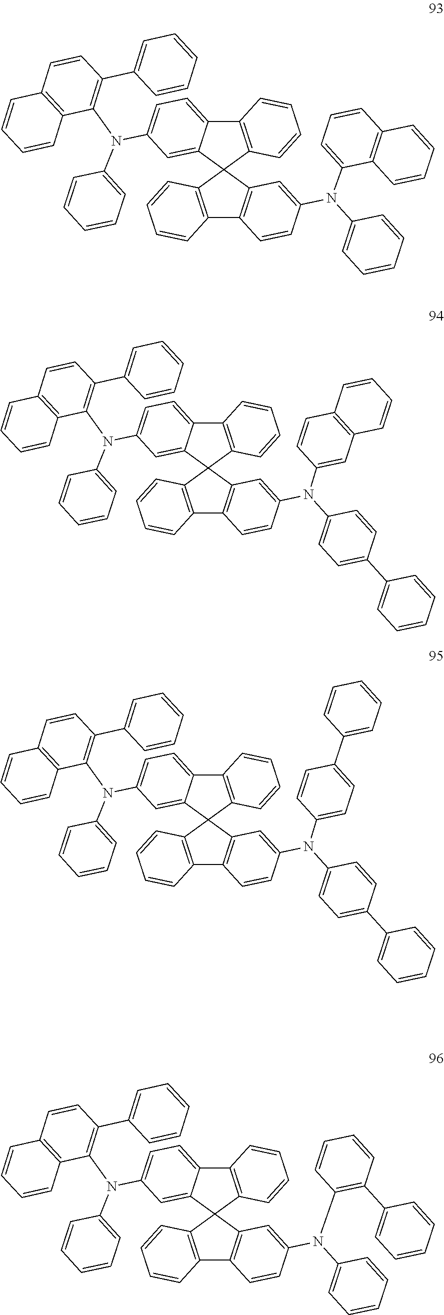 [SMILES: c1ccc(-c2ccc3ccccc3c2N(c2ccccc2)c2ccc3c(c2)C2(c4ccccc4-c4ccc(N(c5ccccc5)c5cccc6ccccc56)cc42)c2ccccc2-3)cc1 ; c1ccc(-c2ccc(N(c3ccc4c(c3)C3(c5ccccc5-4)c4ccccc4-c4ccc(N(c5ccccc5)c5c(-c6ccccc6)ccc6ccccc56)cc43)c3ccc4ccccc4c3)cc2)cc1 ; c1ccc(-c2ccc(N(c3ccc(-c4ccccc4)cc3)c3ccc4c(c3)C3(c5ccccc5-4)c4ccccc4-c4ccc(N(c5ccccc5)c5c(-c6ccccc6)ccc6ccccc56)cc43)cc2)cc1 ; c1ccc(-c2ccccc2N(c2ccccc2)c2ccc3c(c2)C2(c4ccccc4-3)c3ccccc3-c3ccc(N(c4ccccc4)c4c(-c5ccccc5)ccc5ccccc45)cc32)cc1] 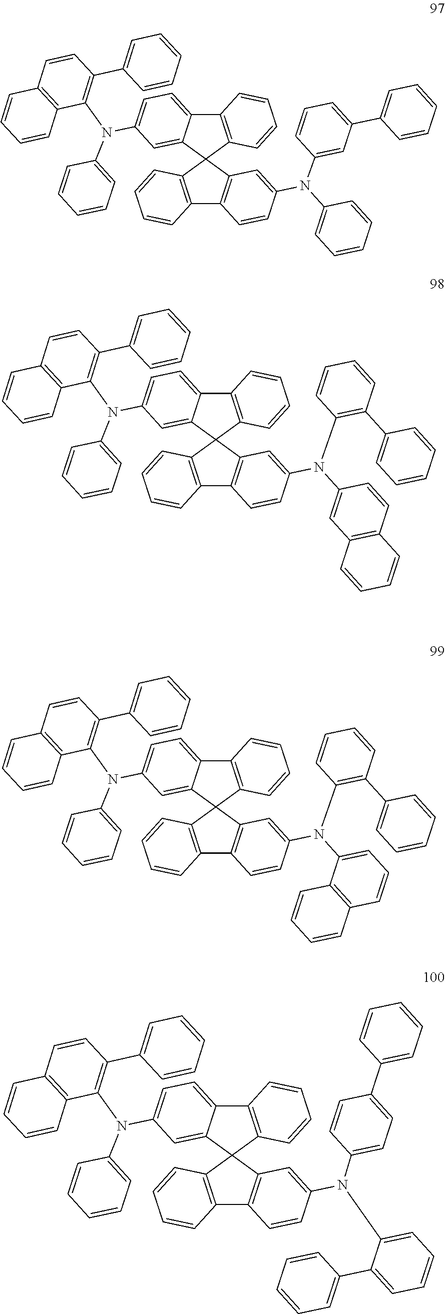 [SMILES: c1ccc(-c2cccc(N(c3ccccc3)c3ccc4c(c3)C3(c5ccccc5-4)c4ccccc4-c4ccc(N(c5ccccc5)c5c(-c6ccccc6)ccc6ccccc56)cc43)c2)cc1 ; c1ccc(-c2ccccc2N(c2ccc3c(c2)C2(c4ccccc4-3)c3ccccc3-c3ccc(N(c4ccccc4)c4c(-c5ccccc5)ccc5ccccc45)cc32)c2ccc3ccccc3c2)cc1 ; c1ccc(-c2ccccc2N(c2ccc3c(c2)C2(c4ccccc4-3)c3ccccc3-c3ccc(N(c4ccccc4)c4c(-c5ccccc5)ccc5ccccc45)cc32)c2cccc3ccccc23)cc1 ; c1ccc(-c2ccc(N(c3ccc4c(c3)C3(c5ccccc5-4)c4ccccc4-c4ccc(N(c5ccccc5)c5c(-c6ccccc6)ccc6ccccc56)cc43)c3ccccc3-c3ccccc3)cc2)cc1] 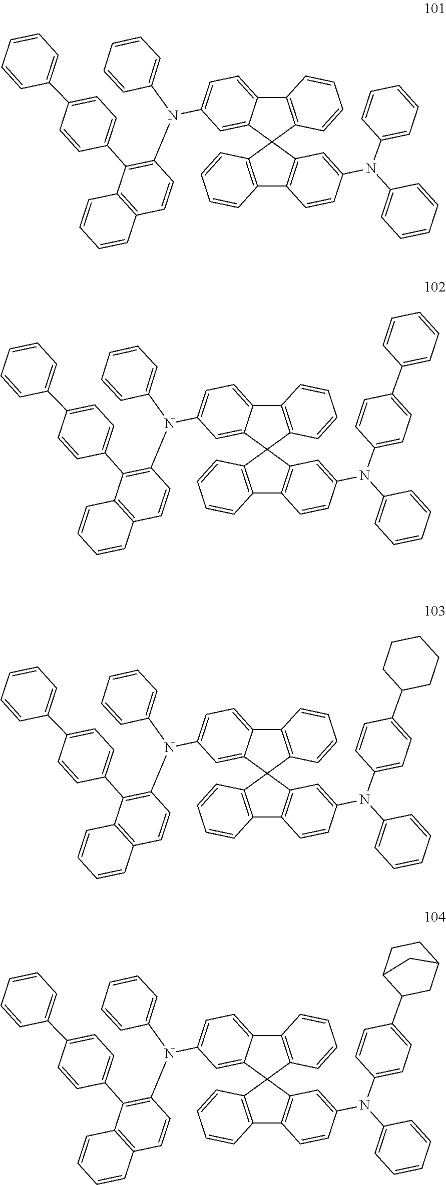 [SMILES: c1ccc(-c2ccc(-c3c(N(c4ccccc4)c4ccc5c(c4)C4(c6ccccc6-c6ccc(N(c7ccccc7)c7ccccc7)cc64)c4ccccc4-5)ccc4ccccc34)cc2)cc1 ; c1ccc(-c2ccc(-c3c(N(c4ccccc4)c4ccc5c(c4)C4(c6ccccc6-c6ccc(N(c7ccccc7)c7ccc(-c8ccccc8)cc7)cc64)c4ccccc4-5)ccc4ccccc34)cc2)cc1 ; c1ccc(-c2ccc(-c3c(N(c4ccccc4)c4ccc5c(c4)C4(c6ccccc6-c6ccc(N(c7ccccc7)c7ccc(C8CCCCC8)cc7)cc64)c4ccccc4-5)ccc4ccccc34)cc2)cc1 ; c1ccc(-c2ccc(-c3c(N(c4ccccc4)c4ccc5c(c4)C4(c6ccccc6-c6ccc(N(c7ccccc7)c7ccc(C8CC9CCC8C9)cc7)cc64)c4ccccc4-5)ccc4ccccc34)cc2)cc1] 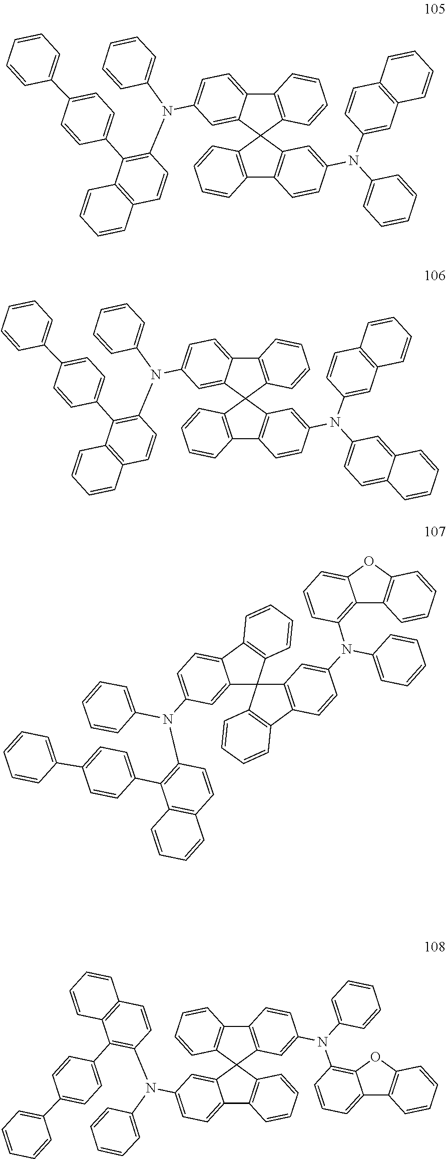 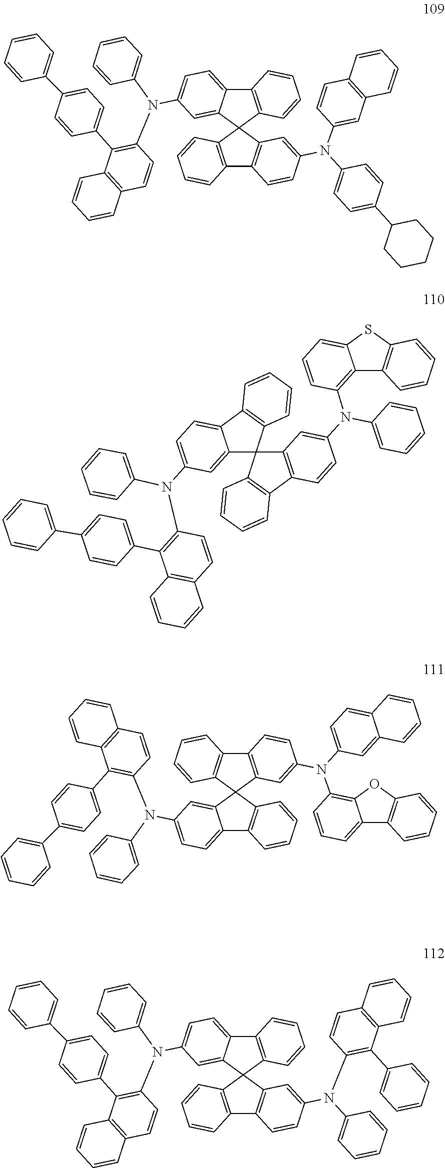 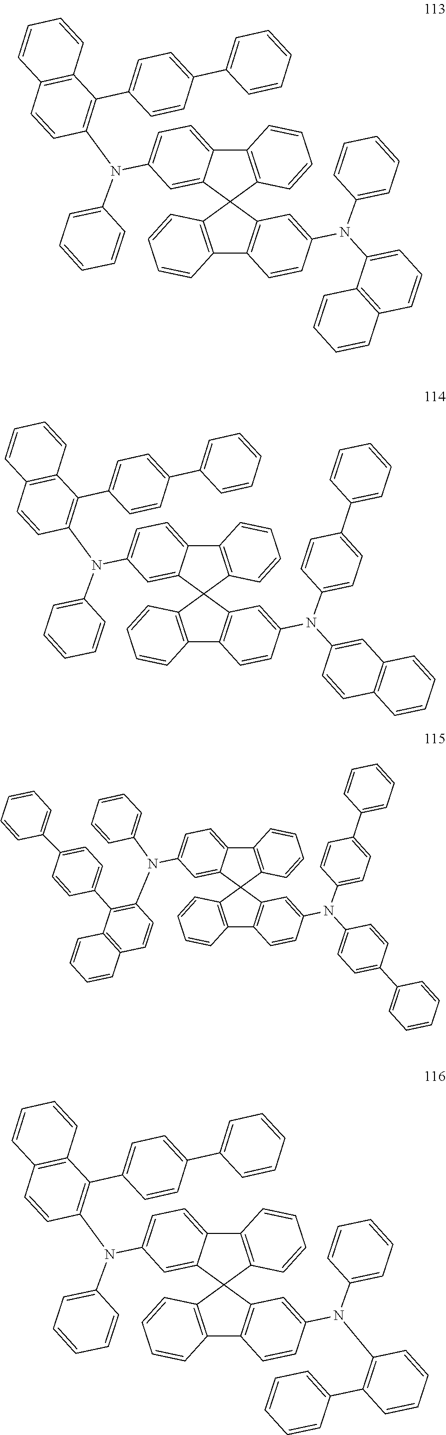 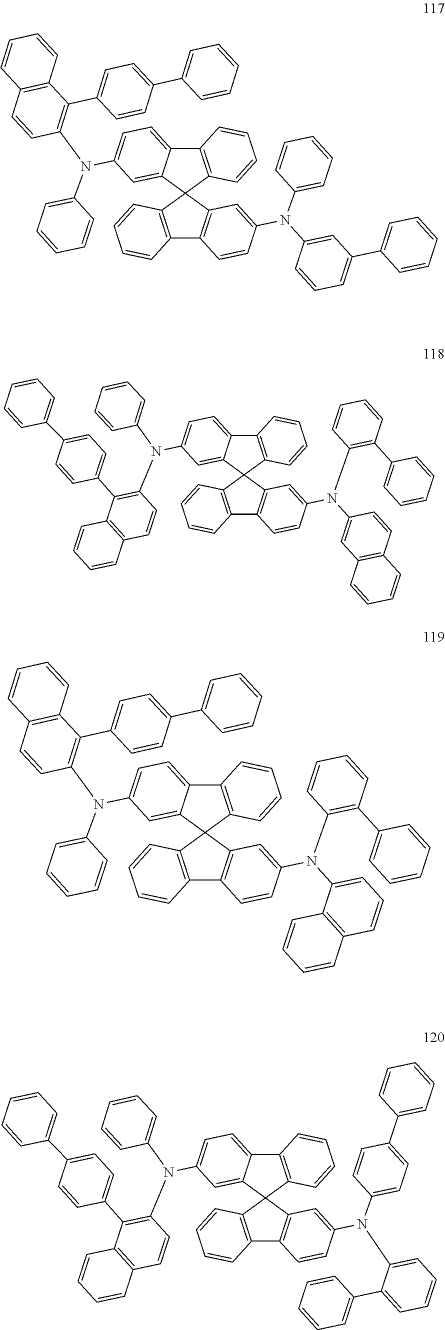 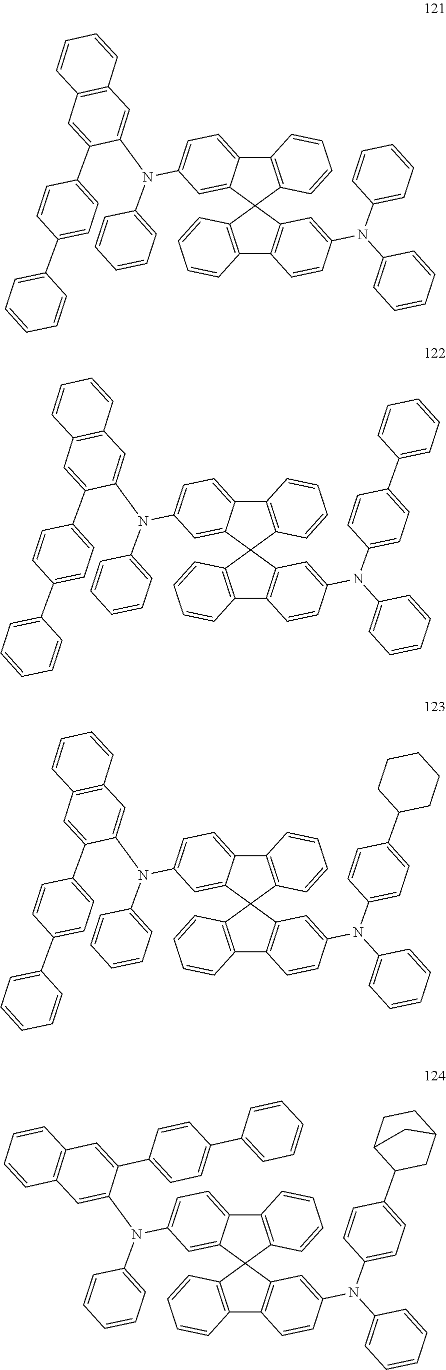 [SMILES: c1ccc(-c2ccc(-c3cc4ccccc4cc3N(c3ccccc3)c3ccc4c(c3)C3(c5ccccc5-c5ccc(N(c6ccccc6)c6ccccc6)cc53)c3ccccc3-4)cc2)cc1 ; c1ccc(-c2ccc(-c3cc4ccccc4cc3N(c3ccccc3)c3ccc4c(c3)C3(c5ccccc5-c5ccc(N(c6ccccc6)c6ccc(-c7ccccc7)cc6)cc53)c3ccccc3-4)cc2)cc1 ; c1ccc(-c2ccc(-c3cc4ccccc4cc3N(c3ccccc3)c3ccc4c(c3)C3(c5ccccc5-c5ccc(N(c6ccccc6)c6ccc(C7CCCCC7)cc6)cc53)c3ccccc3-4)cc2)cc1 ; c1ccc(-c2ccc(-c3cc4ccccc4cc3N(c3ccccc3)c3ccc4c(c3)C3(c5ccccc5-c5ccc(N(c6ccccc6)c6ccc(C7CC8CCC7C8)cc6)cc53)c3ccccc3-4)cc2)cc1] 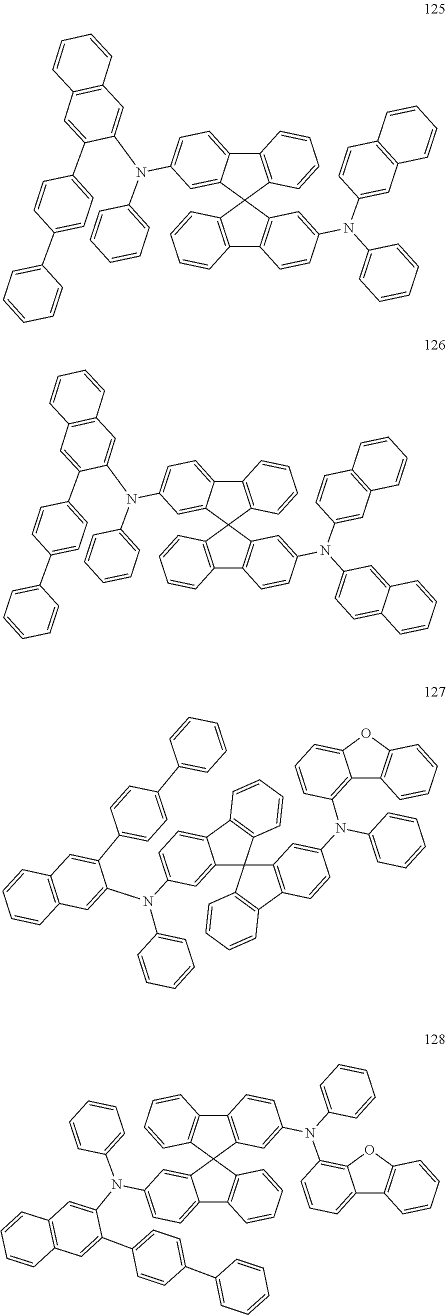 [SMILES: c1ccc(-c2ccc(-c3cc4ccccc4cc3N(c3ccccc3)c3ccc4c(c3)C3(c5ccccc5-c5ccc(N(c6ccccc6)c6ccc7ccccc7c6)cc53)c3ccccc3-4)cc2)cc1 ; c1ccc(-c2ccc(-c3cc4ccccc4cc3N(c3ccccc3)c3ccc4c(c3)C3(c5ccccc5-c5ccc(N(c6ccc7ccccc7c6)c6ccc7ccccc7c6)cc53)c3ccccc3-4)cc2)cc1 ; c1ccc(-c2ccc(-c3cc4ccccc4cc3N(c3ccccc3)c3ccc4c(c3)C3(c5ccccc5-4)c4ccccc4-c4ccc(N(c5ccccc5)c5cccc6oc7ccccc7c56)cc43)cc2)cc1 ; c1ccc(-c2ccc(-c3cc4ccccc4cc3N(c3ccccc3)c3ccc4c(c3)C3(c5ccccc5-4)c4ccccc4-c4ccc(N(c5ccccc5)c5cccc6c5oc5ccccc56)cc43)cc2)cc1] 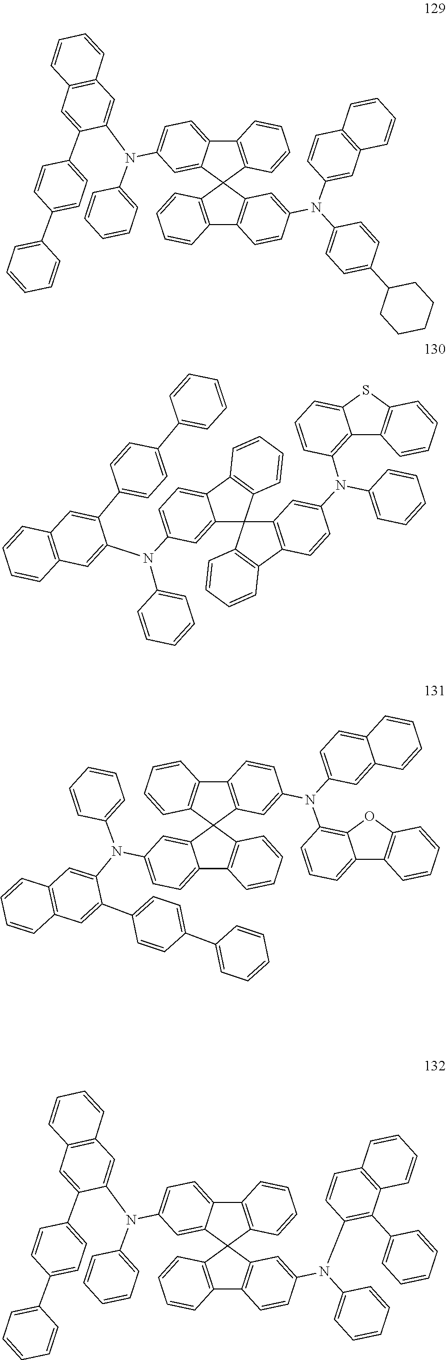 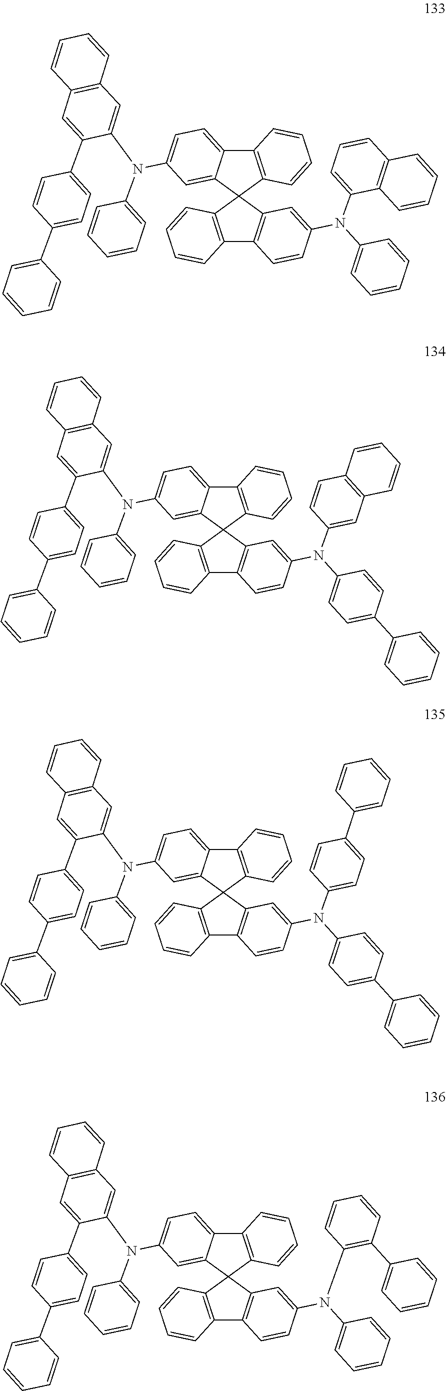 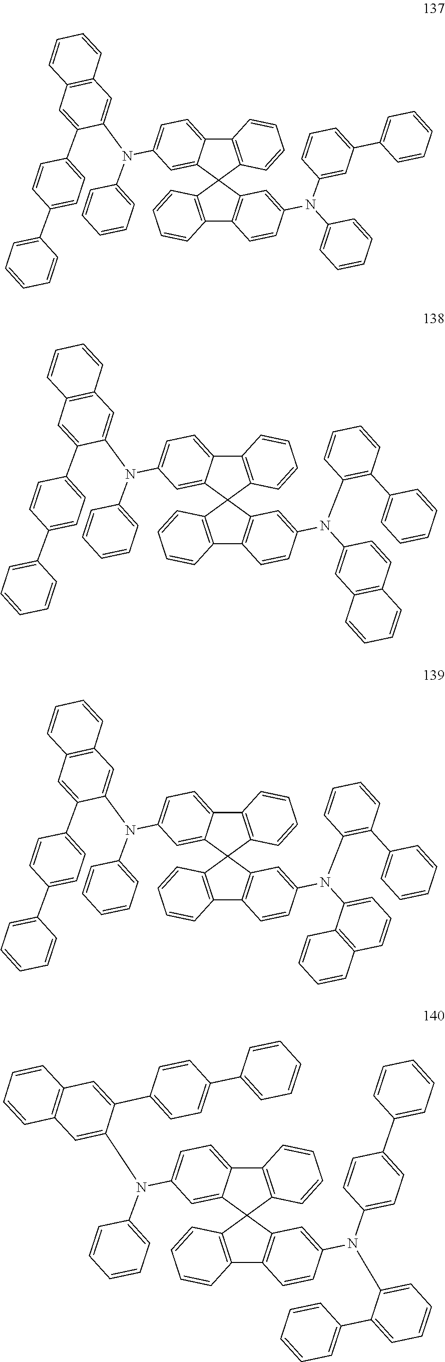 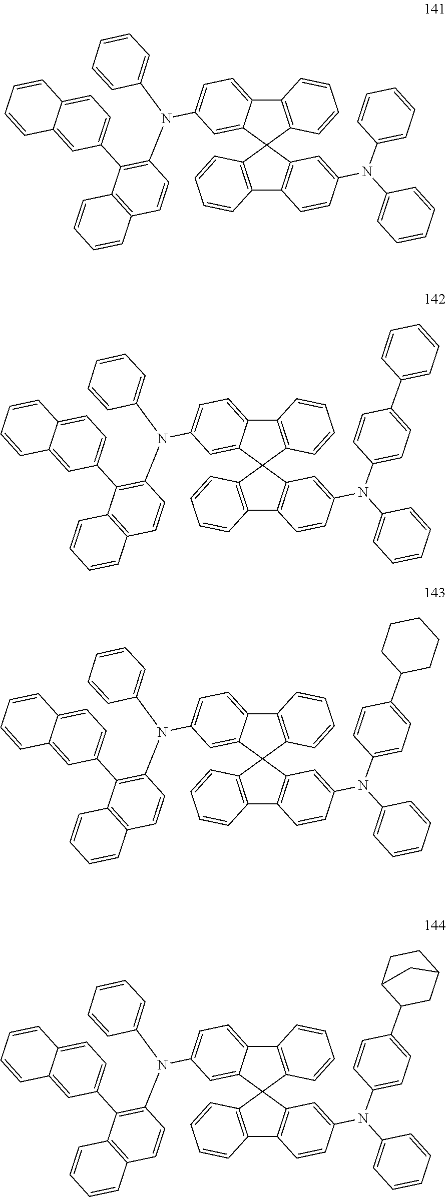 [SMILES: c1ccc(N(c2ccccc2)c2ccc3c(c2)C2(c4ccccc4-3)c3ccccc3-c3ccc(N(c4ccccc4)c4ccc5ccccc5c4-c4ccc5ccccc5c4)cc32)cc1 ; c1ccc(-c2ccc(N(c3ccccc3)c3ccc4c(c3)C3(c5ccccc5-4)c4ccccc4-c4ccc(N(c5ccccc5)c5ccc6ccccc6c5-c5ccc6ccccc6c5)cc43)cc2)cc1 ; c1ccc(N(c2ccc(C3CCCCC3)cc2)c2ccc3c(c2)C2(c4ccccc4-3)c3ccccc3-c3ccc(N(c4ccccc4)c4ccc5ccccc5c4-c4ccc5ccccc5c4)cc32)cc1 ; c1ccc(N(c2ccc(C3CC4CCC3C4)cc2)c2ccc3c(c2)C2(c4ccccc4-3)c3ccccc3-c3ccc(N(c4ccccc4)c4ccc5ccccc5c4-c4ccc5ccccc5c4)cc32)cc1] 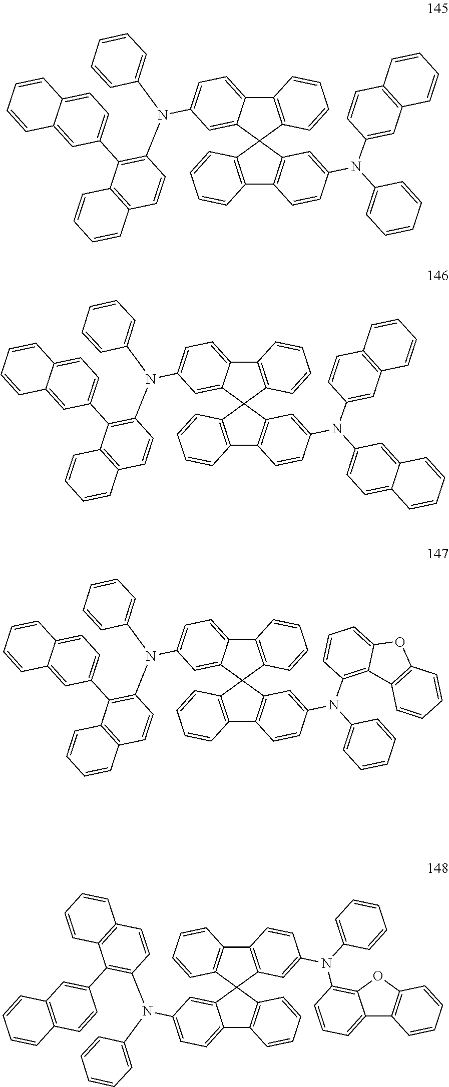 [SMILES: c1ccc(N(c2ccc3c(c2)C2(c4ccccc4-3)c3ccccc3-c3ccc(N(c4ccccc4)c4ccc5ccccc5c4-c4ccc5ccccc5c4)cc32)c2ccc3ccccc3c2)cc1 ; c1ccc(N(c2ccc3c(c2)C2(c4ccccc4-c4ccc(N(c5ccc6ccccc6c5)c5ccc6ccccc6c5)cc42)c2ccccc2-3)c2ccc3ccccc3c2-c2ccc3ccccc3c2)cc1 ; c1ccc(N(c2ccc3c(c2)C2(c4ccccc4-3)c3ccccc3-c3ccc(N(c4ccccc4)c4cccc5oc6ccccc6c45)cc32)c2ccc3ccccc3c2-c2ccc3ccccc3c2)cc1 ; c1ccc(N(c2ccc3c(c2)C2(c4ccccc4-3)c3ccccc3-c3ccc(N(c4ccccc4)c4cccc5c4oc4ccccc45)cc32)c2ccc3ccccc3c2-c2ccc3ccccc3c2)cc1] 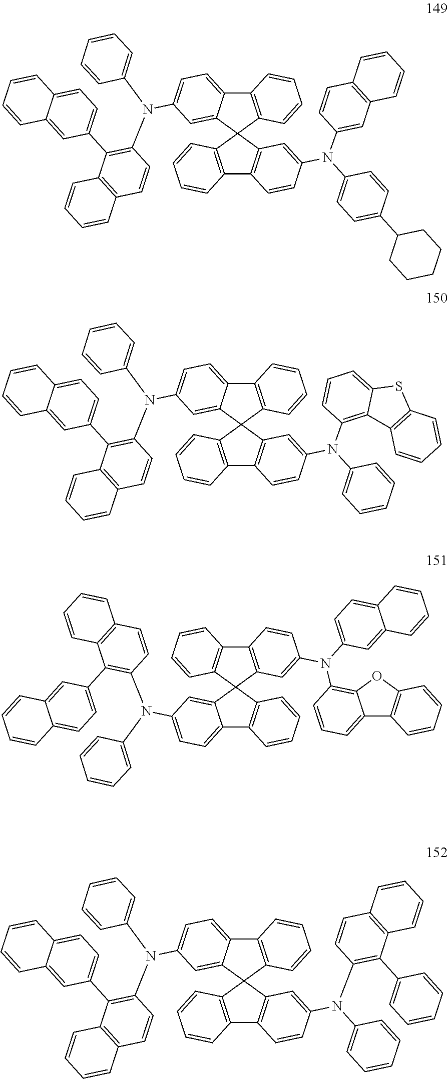 [SMILES: c1ccc(N(c2ccc3c(c2)C2(c4ccccc4-c4ccc(N(c5ccc(C6CCCCC6)cc5)c5ccc6ccccc6c5)cc42)c2ccccc2-3)c2ccc3ccccc3c2-c2ccc3ccccc3c2)cc1 ; c1ccc(N(c2ccc3c(c2)C2(c4ccccc4-3)c3ccccc3-c3ccc(N(c4ccccc4)c4cccc5sc6ccccc6c45)cc32)c2ccc3ccccc3c2-c2ccc3ccccc3c2)cc1 ; c1ccc(N(c2ccc3c(c2)C2(c4ccccc4-3)c3ccccc3-c3ccc(N(c4ccc5ccccc5c4)c4cccc5c4oc4ccccc45)cc32)c2ccc3ccccc3c2-c2ccc3ccccc3c2)cc1 ; c1ccc(-c2c(N(c3ccccc3)c3ccc4c(c3)C3(c5ccccc5-4)c4ccccc4-c4ccc(N(c5ccccc5)c5ccc6ccccc6c5-c5ccc6ccccc6c5)cc43)ccc3ccccc23)cc1] 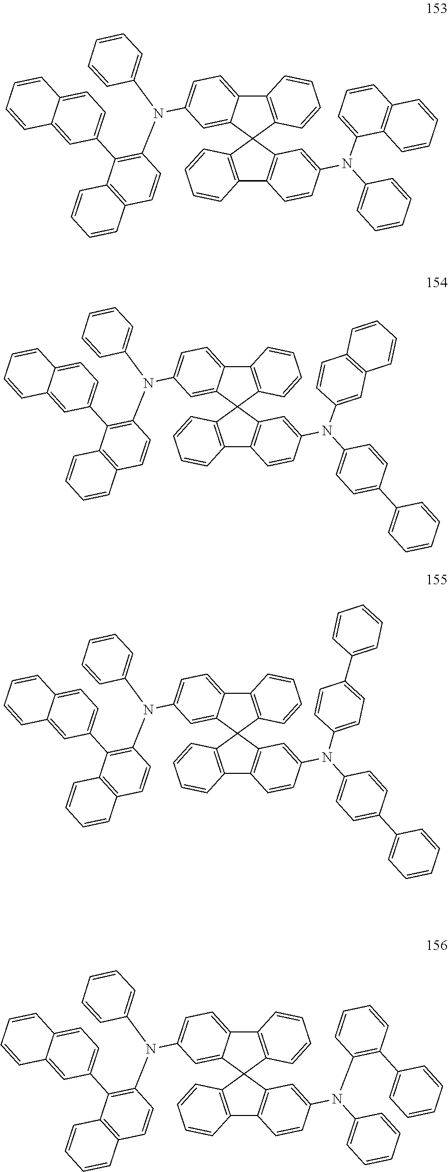 [SMILES: c1ccc(N(c2ccc3c(c2)C2(c4ccccc4-3)c3ccccc3-c3ccc(N(c4ccccc4)c4cccc5ccccc45)cc32)c2ccc3ccccc3c2-c2ccc3ccccc3c2)cc1 ; c1ccc(-c2ccc(N(c3ccc4c(c3)C3(c5ccccc5-4)c4ccccc4-c4ccc(N(c5ccccc5)c5ccc6ccccc6c5-c5ccc6ccccc6c5)cc43)c3ccc4ccccc4c3)cc2)cc1 ; c1ccc(-c2ccc(N(c3ccc(-c4ccccc4)cc3)c3ccc4c(c3)C3(c5ccccc5-4)c4ccccc4-c4ccc(N(c5ccccc5)c5ccc6ccccc6c5-c5ccc6ccccc6c5)cc43)cc2)cc1 ; c1ccc(-c2ccccc2N(c2ccccc2)c2ccc3c(c2)C2(c4ccccc4-3)c3ccccc3-c3ccc(N(c4ccccc4)c4ccc5ccccc5c4-c4ccc5ccccc5c4)cc32)cc1] 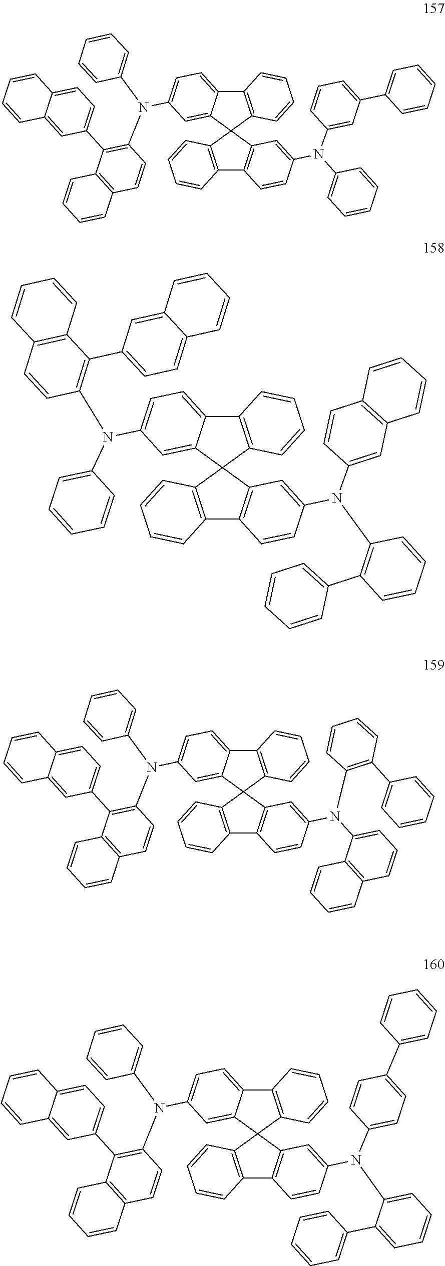 [SMILES: c1ccc(-c2cccc(N(c3ccccc3)c3ccc4c(c3)C3(c5ccccc5-4)c4ccccc4-c4ccc(N(c5ccccc5)c5ccc6ccccc6c5-c5ccc6ccccc6c5)cc43)c2)cc1 ; c1ccc(-c2ccccc2N(c2ccc3c(c2)C2(c4ccccc4-3)c3ccccc3-c3ccc(N(c4ccccc4)c4ccc5ccccc5c4-c4ccc5ccccc5c4)cc32)c2ccc3ccccc3c2)cc1 ; c1ccc(-c2ccccc2N(c2ccc3c(c2)C2(c4ccccc4-c4ccc(N(c5ccccc5)c5ccc6ccccc6c5-c5ccc6ccccc6c5)cc42)c2ccccc2-3)c2cccc3ccccc23)cc1 ; c1ccc(-c2ccc(N(c3ccc4c(c3)C3(c5ccccc5-4)c4ccccc4-c4ccc(N(c5ccccc5)c5ccc6ccccc6c5-c5ccc6ccccc6c5)cc43)c3ccccc3-c3ccccc3)cc2)cc1] 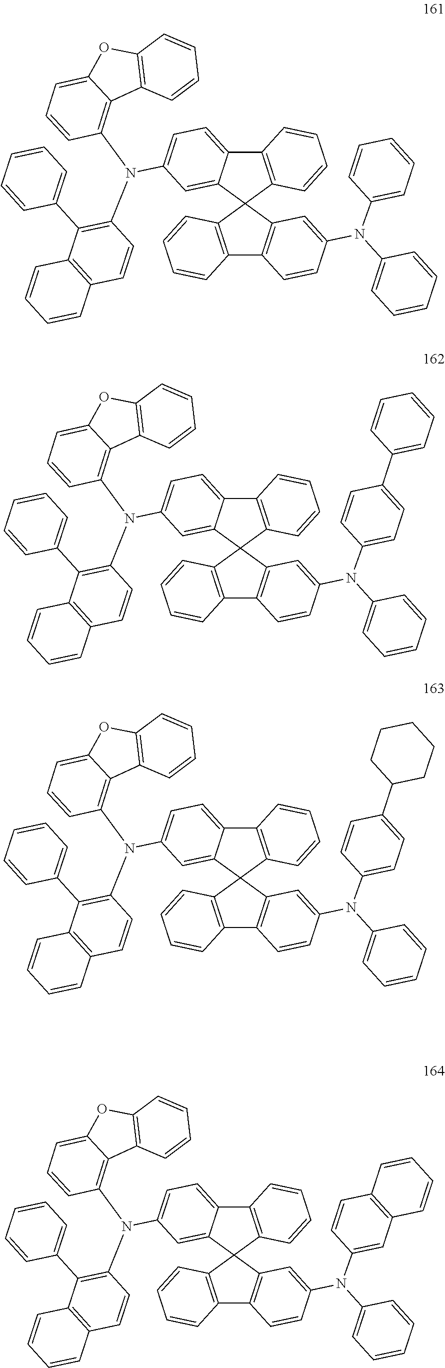 [SMILES: c1ccc(-c2c(N(c3ccc4c(c3)C3(c5ccccc5-c5ccc(N(c6ccccc6)c6ccccc6)cc53)c3ccccc3-4)c3cccc4oc5ccccc5c34)ccc3ccccc23)cc1 ; c1ccc(-c2ccc(N(c3ccccc3)c3ccc4c(c3)C3(c5ccccc5-4)c4ccccc4-c4ccc(N(c5ccc6ccccc6c5-c5ccccc5)c5cccc6oc7ccccc7c56)cc43)cc2)cc1 ; c1ccc(-c2c(N(c3ccc4c(c3)C3(c5ccccc5-c5ccc(N(c6ccccc6)c6ccc(C7CCCCC7)cc6)cc53)c3ccccc3-4)c3cccc4oc5ccccc5c34)ccc3ccccc23)cc1 ; c1ccc(-c2c(N(c3ccc4c(c3)C3(c5ccccc5-c5ccc(N(c6ccccc6)c6ccc7ccccc7c6)cc53)c3ccccc3-4)c3cccc4oc5ccccc5c34)ccc3ccccc23)cc1] 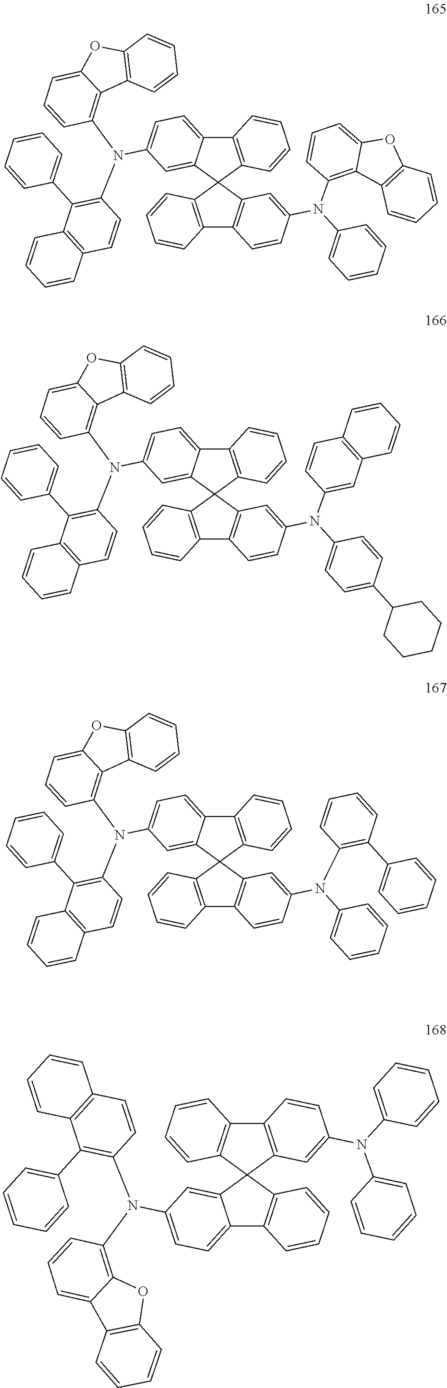 [SMILES: c1ccc(-c2c(N(c3ccc4c(c3)C3(c5ccccc5-c5ccc(N(c6ccccc6)c6cccc7oc8ccccc8c67)cc53)c3ccccc3-4)c3cccc4oc5ccccc5c34)ccc3ccccc23)cc1 ; c1ccc(-c2c(N(c3ccc4c(c3)C3(c5ccccc5-c5ccc(N(c6ccc(C7CCCCC7)cc6)c6ccc7ccccc7c6)cc53)c3ccccc3-4)c3cccc4oc5ccccc5c34)ccc3ccccc23)cc1 ; c1ccc(-c2ccccc2N(c2ccccc2)c2ccc3c(c2)C2(c4ccccc4-3)c3ccccc3-c3ccc(N(c4ccc5ccccc5c4-c4ccccc4)c4cccc5oc6ccccc6c45)cc32)cc1 ; c1ccc(-c2c(N(c3ccc4c(c3)C3(c5ccccc5-c5ccc(N(c6ccccc6)c6ccccc6)cc53)c3ccccc3-4)c3cccc4c3oc3ccccc34)ccc3ccccc23)cc1] 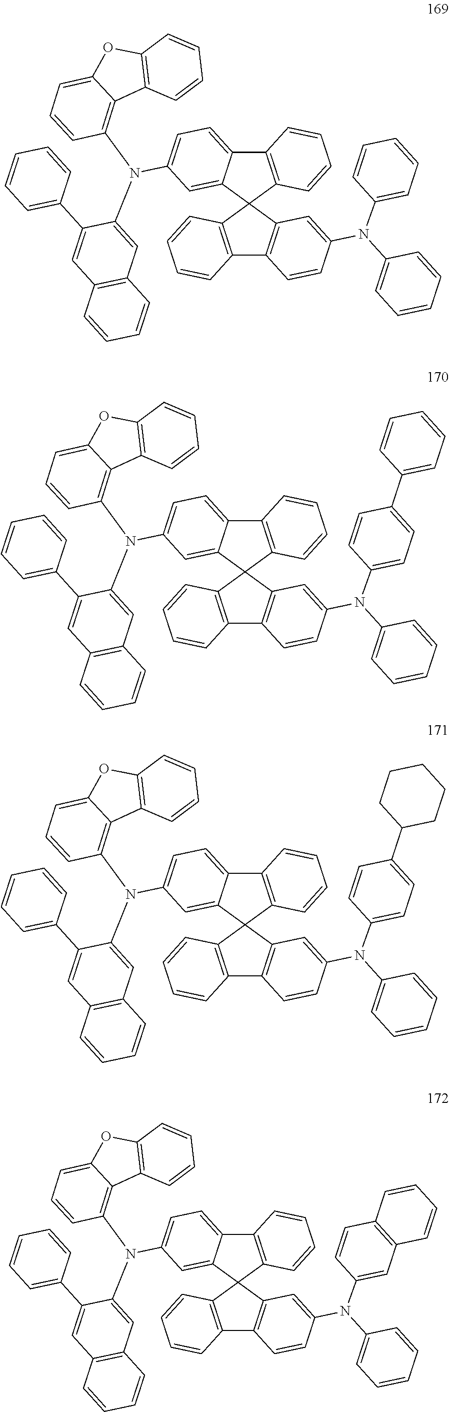 [SMILES: c1ccc(-c2cc3ccccc3cc2N(c2ccc3c(c2)C2(c4ccccc4-c4ccc(N(c5ccccc5)c5ccccc5)cc42)c2ccccc2-3)c2cccc3oc4ccccc4c23)cc1 ; c1ccc(-c2ccc(N(c3ccccc3)c3ccc4c(c3)C3(c5ccccc5-4)c4ccccc4-c4ccc(N(c5cc6ccccc6cc5-c5ccccc5)c5cccc6oc7ccccc7c56)cc43)cc2)cc1 ; c1ccc(-c2cc3ccccc3cc2N(c2ccc3c(c2)C2(c4ccccc4-c4ccc(N(c5ccccc5)c5ccc(C6CCCCC6)cc5)cc42)c2ccccc2-3)c2cccc3oc4ccccc4c23)cc1 ; c1ccc(-c2cc3ccccc3cc2N(c2ccc3c(c2)C2(c4ccccc4-c4ccc(N(c5ccccc5)c5ccc6ccccc6c5)cc42)c2ccccc2-3)c2cccc3oc4ccccc4c23)cc1] 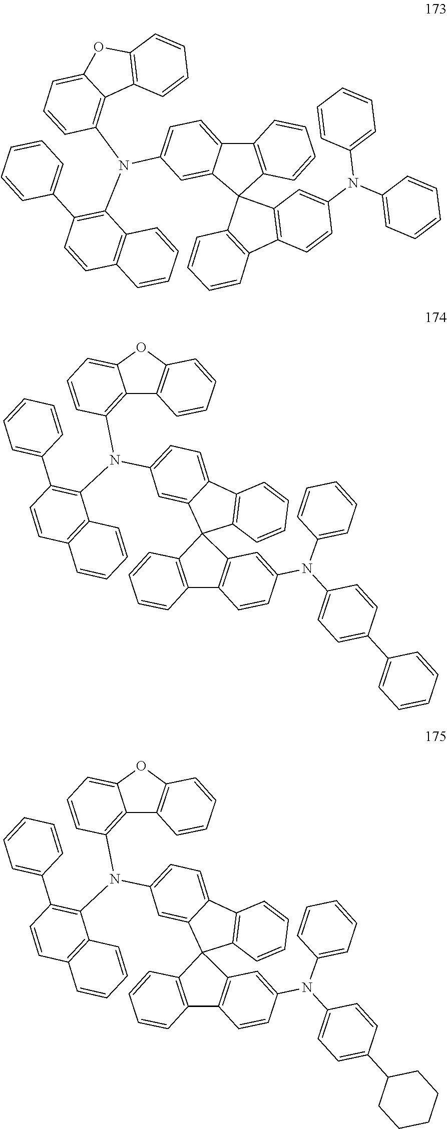 [SMILES: c1ccc(-c2ccc3ccccc3c2N(c2ccc3c(c2)C2(c4ccccc4-c4ccc(N(c5ccccc5)c5ccccc5)cc42)c2ccccc2-3)c2cccc3oc4ccccc4c23)cc1 ; c1ccc(-c2ccc(N(c3ccccc3)c3ccc4c(c3)C3(c5ccccc5-4)c4ccccc4-c4ccc(N(c5c(-c6ccccc6)ccc6ccccc56)c5cccc6oc7ccccc7c56)cc43)cc2)cc1 ; c1ccc(-c2ccc3ccccc3c2N(c2ccc3c(c2)C2(c4ccccc4-c4ccc(N(c5ccccc5)c5ccc(C6CCCCC6)cc5)cc42)c2ccccc2-3)c2cccc3oc4ccccc4c23)cc1] 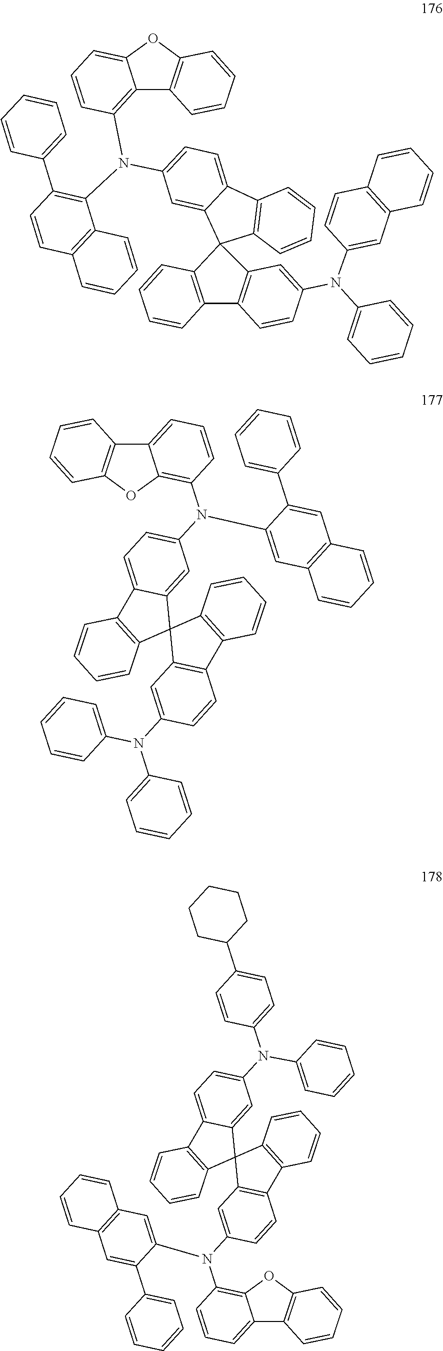 [SMILES: c1ccc(-c2ccc3ccccc3c2N(c2ccc3c(c2)C2(c4ccccc4-c4ccc(N(c5ccccc5)c5ccc6ccccc6c5)cc42)c2ccccc2-3)c2cccc3oc4ccccc4c23)cc1 ; c1ccc(-c2cc3ccccc3cc2N(c2ccc3c(c2)C2(c4ccccc4-c4ccc(N(c5ccccc5)c5ccccc5)cc42)c2ccccc2-3)c2cccc3c2oc2ccccc23)cc1 ; c1ccc(-c2cc3ccccc3cc2N(c2ccc3c(c2)C2(c4ccccc4-c4ccc(N(c5ccccc5)c5ccc(C6CCCCC6)cc5)cc42)c2ccccc2-3)c2cccc3c2oc2ccccc23)cc1] 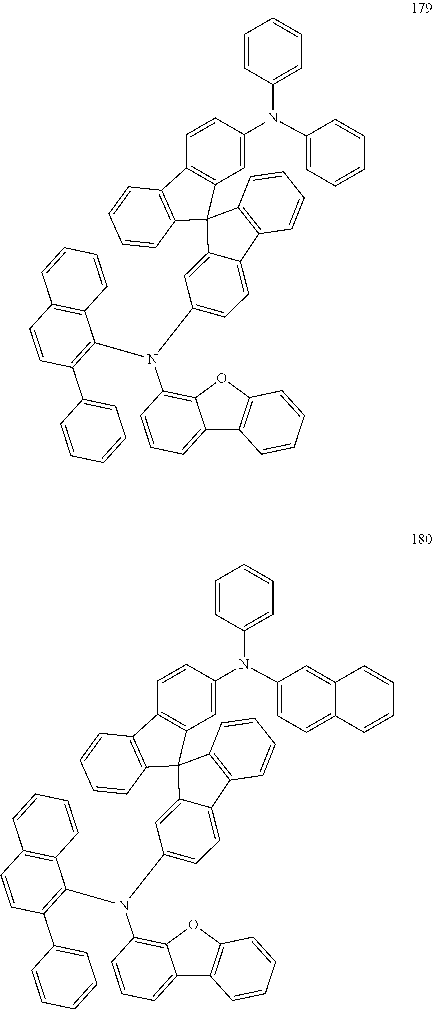 [SMILES: c1ccc(-c2ccc3ccccc3c2N(c2ccc3c(c2)C2(c4ccccc4-c4ccc(N(c5ccccc5)c5ccccc5)cc42)c2ccccc2-3)c2cccc3c2oc2ccccc23)cc1 ; c1ccc(-c2ccc3ccccc3c2N(c2ccc3c(c2)C2(c4ccccc4-c4ccc(N(c5ccccc5)c5ccc6ccccc6c5)cc42)c2ccccc2-3)c2cccc3c2oc2ccccc23)cc1] 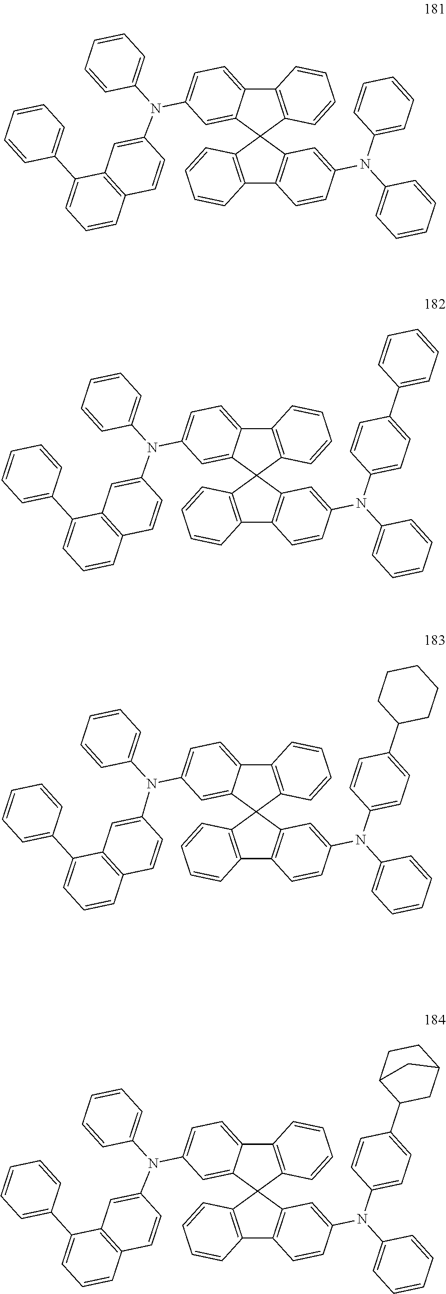 [SMILES: c1ccc(-c2cccc3ccc(N(c4ccccc4)c4ccc5c(c4)C4(c6ccccc6-c6ccc(N(c7ccccc7)c7ccccc7)cc64)c4ccccc4-5)cc23)cc1 ; c1ccc(-c2ccc(N(c3ccccc3)c3ccc4c(c3)C3(c5ccccc5-4)c4ccccc4-c4ccc(N(c5ccccc5)c5ccc6cccc(-c7ccccc7)c6c5)cc43)cc2)cc1 ; c1ccc(-c2cccc3ccc(N(c4ccccc4)c4ccc5c(c4)C4(c6ccccc6-c6ccc(N(c7ccccc7)c7ccc(C8CCCCC8)cc7)cc64)c4ccccc4-5)cc23)cc1 ; c1ccc(-c2cccc3ccc(N(c4ccccc4)c4ccc5c(c4)C4(c6ccccc6-c6ccc(N(c7ccccc7)c7ccc(C8CC9CCC8C9)cc7)cc64)c4ccccc4-5)cc23)cc1] 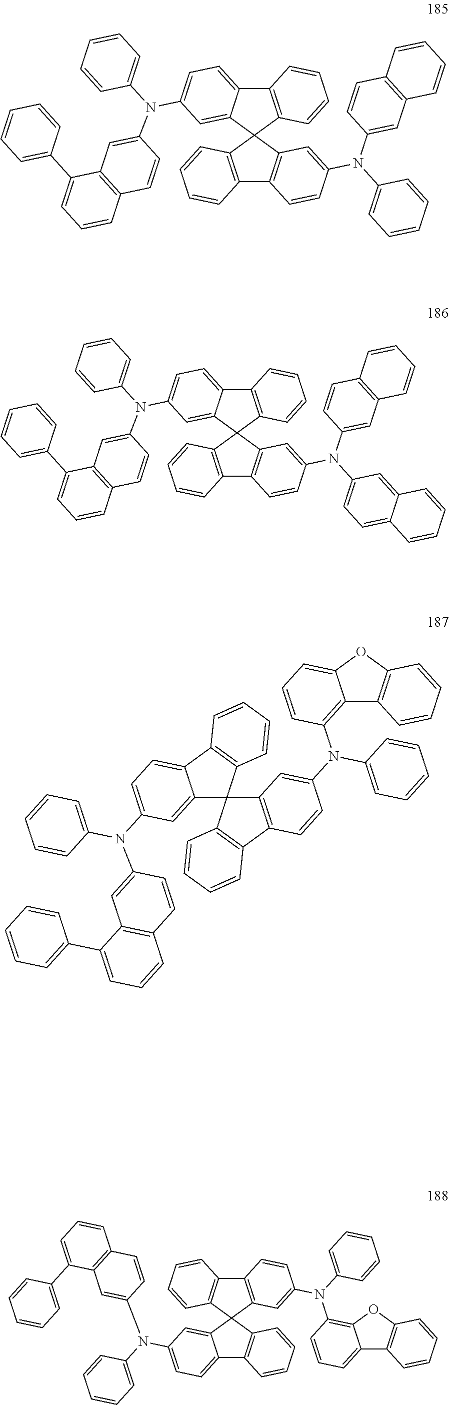 [SMILES: c1ccc(-c2cccc3ccc(N(c4ccccc4)c4ccc5c(c4)C4(c6ccccc6-c6ccc(N(c7ccccc7)c7ccc8ccccc8c7)cc64)c4ccccc4-5)cc23)cc1 ; c1ccc(-c2cccc3ccc(N(c4ccccc4)c4ccc5c(c4)C4(c6ccccc6-c6ccc(N(c7ccc8ccccc8c7)c7ccc8ccccc8c7)cc64)c4ccccc4-5)cc23)cc1 ; c1ccc(-c2cccc3ccc(N(c4ccccc4)c4ccc5c(c4)C4(c6ccccc6-5)c5ccccc5-c5ccc(N(c6ccccc6)c6cccc7oc8ccccc8c67)cc54)cc23)cc1 ; c1ccc(-c2cccc3ccc(N(c4ccccc4)c4ccc5c(c4)C4(c6ccccc6-5)c5ccccc5-c5ccc(N(c6ccccc6)c6cccc7c6oc6ccccc67)cc54)cc23)cc1] 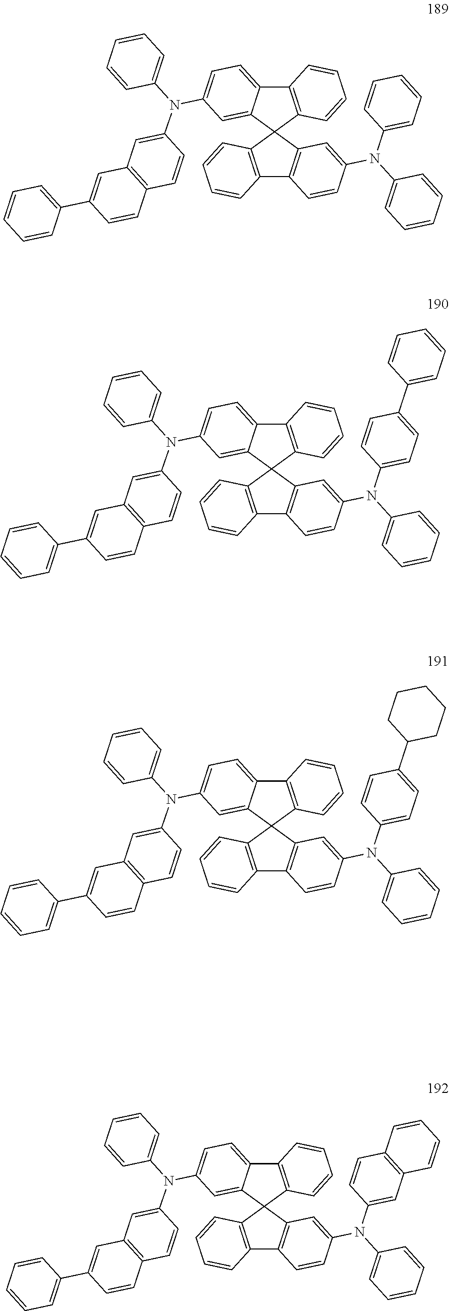 [SMILES: c1ccc(-c2ccc3ccc(N(c4ccccc4)c4ccc5c(c4)C4(c6ccccc6-c6ccc(N(c7ccccc7)c7ccccc7)cc64)c4ccccc4-5)cc3c2)cc1 ; c1ccc(-c2ccc(N(c3ccccc3)c3ccc4c(c3)C3(c5ccccc5-4)c4ccccc4-c4ccc(N(c5ccccc5)c5ccc6ccc(-c7ccccc7)cc6c5)cc43)cc2)cc1 ; c1ccc(-c2ccc3ccc(N(c4ccccc4)c4ccc5c(c4)C4(c6ccccc6-c6ccc(N(c7ccccc7)c7ccc(C8CCCCC8)cc7)cc64)c4ccccc4-5)cc3c2)cc1 ; c1ccc(-c2ccc3ccc(N(c4ccccc4)c4ccc5c(c4)C4(c6ccccc6-c6ccc(N(c7ccccc7)c7ccc8ccccc8c7)cc64)c4ccccc4-5)cc3c2)cc1] 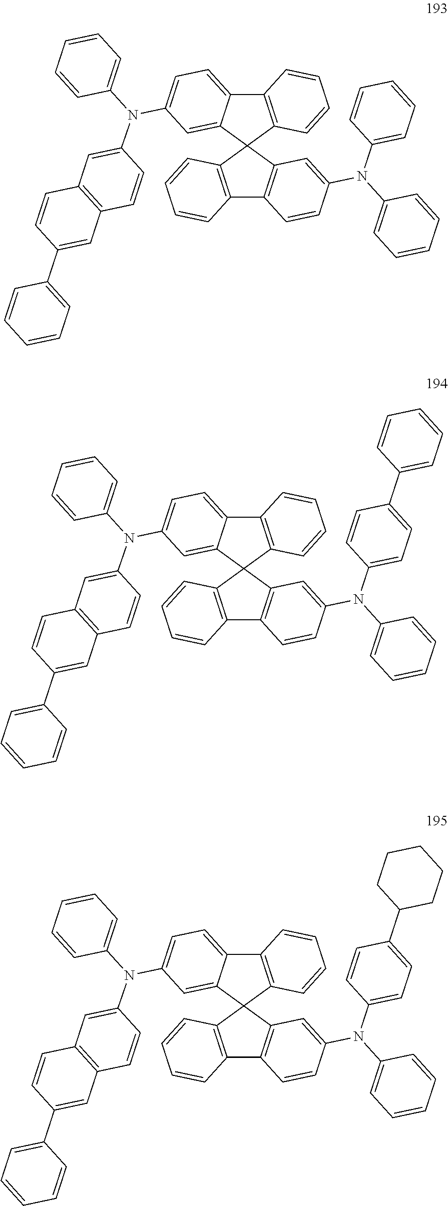 [SMILES: c1ccc(-c2ccc3cc(N(c4ccccc4)c4ccc5c(c4)C4(c6ccccc6-c6ccc(N(c7ccccc7)c7ccccc7)cc64)c4ccccc4-5)ccc3c2)cc1 ; c1ccc(-c2ccc(N(c3ccccc3)c3ccc4c(c3)C3(c5ccccc5-4)c4ccccc4-c4ccc(N(c5ccccc5)c5ccc6cc(-c7ccccc7)ccc6c5)cc43)cc2)cc1 ; c1ccc(-c2ccc3cc(N(c4ccccc4)c4ccc5c(c4)C4(c6ccccc6-c6ccc(N(c7ccccc7)c7ccc(C8CCCCC8)cc7)cc64)c4ccccc4-5)ccc3c2)cc1] 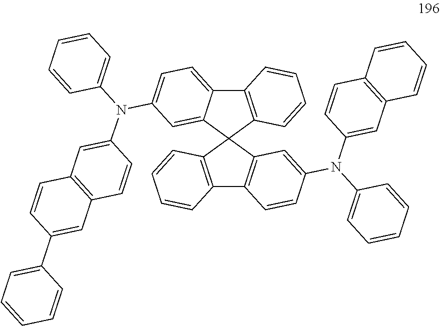 [SMILES: c1ccc(-c2ccc3cc(N(c4ccccc4)c4ccc5c(c4)C4(c6ccccc6-c6ccc(N(c7ccccc7)c7ccc8ccccc8c7)cc64)c4ccccc4-5)ccc3c2)cc1] 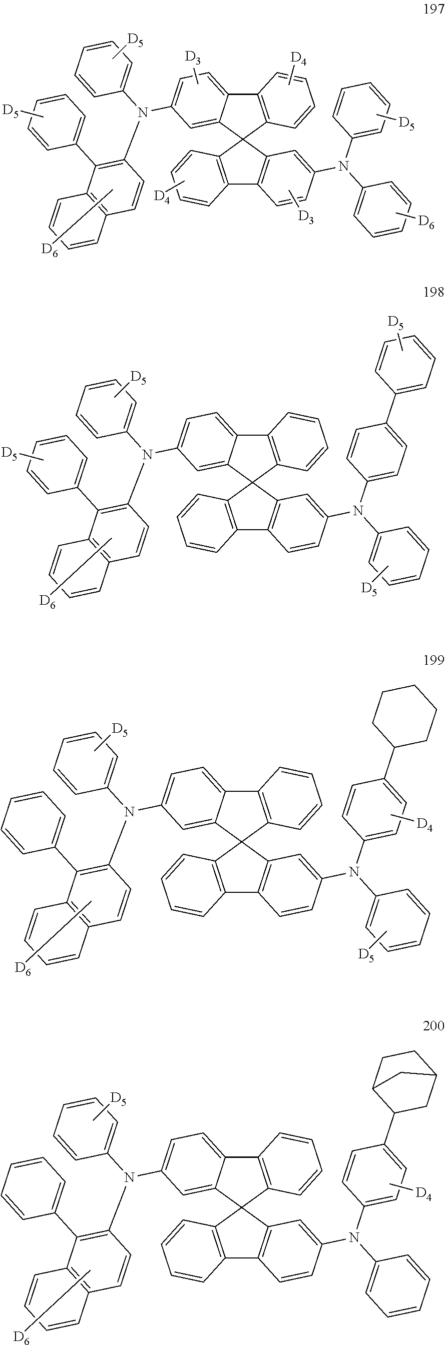 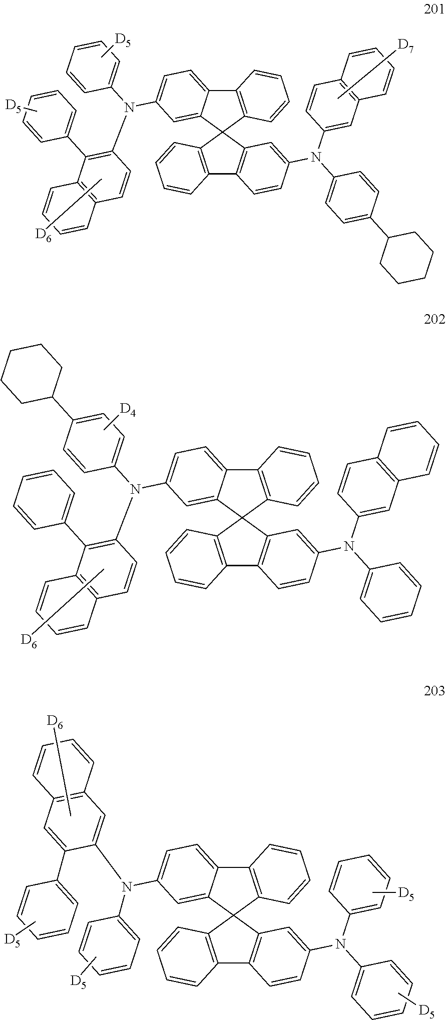 [chemical structure]
